(19) United States
(12) Patent Application Publication
(10) Pub. No.: US 2024/0368664 A1
(43) Pub. Date: Nov. 7, 2024

(54) LIVE YEAST BIOSENSORS AND METHODS OF USE THEREOF

(71) Applicant: THE TRUSTEES OF COLUMBIA UNIVERSITY IN THE CITY OF NEW YORK, New York, NY (US)

(72) Inventors: Virginia Cornish, New York, NY (US); Tea CRNKOVIC, Chelsea, MA (US); Davida RIOS, New York, NY (US); Amirhossein JAFARIYAN, New York, NY (US); Marco FANTINI, New York, NY (US); Anton KOZYRYEV, White Plains, NY (US)

(73) Assignee: THE TRUSTEES OF COLUMBIA UNIVERSITY IN THE CITY OF NEW YORK, New York, NY (US)

(21) Appl. No.: 18/513,135

(22) Filed: Nov. 17, 2023

Related U.S. Application Data
(63) Continuation of application No. PCT/US2022/029914, filed on May 18, 2022.
(60) Provisional application No. 63/296,771, filed on Jan. 5, 2022.
(60) Provisional application No. 63/228,577, filed on Aug. 2, 2021.
(60) Provisional application No. 63/228,547, filed on Aug. 2, 2021.
(60) Provisional application No. 63/190,039, filed on May 18, 2021.

(51) Int. Cl. C12Q 1/02; G01N 33/566; G01N 33/569; G01N 33/68

(52) U.S. Cl. CPC C12Q 1/02; G01N 33/566; G01N 33/56961; G01N 33/56983; G01N 33/68; G01N 2333/165; G01N 2333/38; G01N 2333/39; G01N 2333/4719

(57) ABSTRACT

The present disclosure relates to kits, compositions and methods for detecting fungal species, viruses and/or protein variants in a sample, e.g., a biological sample. For example, but not by way of limitation, the present disclosure provides living yeast biosensors that have been genetically engineered to detect fungal species, viruses and/or protein variants in a sample, e.g., a biological sample.

### CROSS-REFERENCE TO RELATED APPLICATIONS

This application is a continuation of International Patent Application No. PCT/US2022/029914, filed May 18, 2022, which claims the benefit of U.S. Provisional Application No. 63/190,039, filed May 18, 2021, U.S. Provisional Application No. 63/228,547, filed Aug. 2, 2021, U.S. Provisional Application No. 63/228,577, filed Aug. 2, 2021, and U.S. Provisional Application No. 63/296,771, filed Jan. 5, 2022, the contents of each of which are hereby incorporated by reference in their entirety herein, and to each of which priority is claimed.

### GRANT INFORMATION

This invention was made with government support under grants AI110794 and R01AI110794-01A1 awarded by the National Institutes of Health and under grants HR0011-17-C-0068 and HR0011-15-2-0032 awarded by DARPA. The government has certain rights in the invention.

### SEQUENCE LISTING

The instant application contains a Sequence Listing which has been submitted in XML format via EFS-Web and is hereby incorporated by reference in its entirety. Said XML copy, created on Nov. 17, 2023, is named 070050_6674.xml and is 614,521 bytes in size. The Sequence Listing, electronically filed herewith, does not extend beyond the scope of the specification and thus does not contain new matter.

### INTRODUCTION

The present disclosure relates to kits, compositions and methods for detecting fungal species, viruses and/or protein variants in a sample, e.g., a biological sample. For example, but not by way of limitation, the present disclosure provides living yeast biosensors that have been genetically engineered to detect fungal species, viruses and/or protein variants in a sample, e.g., a biological sample.

In certain embodiments, the present disclosure provides kits, compositions and methods for detecting a fungal species in a sample. In certain embodiments, the present disclosure provides compositions and methods for detecting aspergillosis, e.g., by detecting species of the Aspergillus genus, e.g., Aspergillus fumigatus (A. fumigatus), in a sample.

In certain embodiments, the present disclosure provides kits, compositions and methods for detecting a virus in a sample, e.g., a respiratory virus, a hemorrhagic virus, a gastrointestinal virus, an exanthematous virus, a hepatitis virus, a sexually transmitted virus and/or a neurological virus. In certain embodiments, the present disclosure provides compositions and methods for detecting a respiratory virus, e.g., a coronavirus (e.g., SARS-CoV-2), by detecting an analyte derived from the respiratory virus in a sample. In certain embodiments, the present disclosure provides compositions and methods for detecting a hemorrhagic virus, e.g., an ebolavirus, by detecting an analyte derived from the hemorrhagic virus in a sample.

The present disclosure further provides kits, compositions and methods for detecting variants of a polypeptide, e.g., a variant of a protein, in a sample. In certain embodiments, the present disclosure provides compositions and methods for detecting a protein variant, e.g., a variant of a viral protein, in a sample.

### BACKGROUND

Viral diseases and fungal infections kill millions of people worldwide every year. For example, fungal infections are a persisting global health problem that result in over 1.5 million annual deaths worldwide. There is an increasing number of susceptible populations to fungal diseases, such as those having tuberculosis, chronic obstructive pulmonary disease (COPD), asthma, cancers and, even coronavirus disease 2019 (COVID-19) patients. Similarly, viral diseases kill over 1.5 million and infect over 1.5 billion every year; of these about 1.1 billion infections and 1.4 million deaths occur in resource-poor countries.

With the growing incidence of such diseases and infections, timely and accurate diagnosis has become paramount in order to start early treatment which substantially improves patient clinical outcomes and prevents further outbreaks. One common fungal genus to cause invasive fungal infections is Aspergillus. Within this genus, A. fumigatus is the most common species that causes invasive aspergillosis and allergic disease. Continuous exposure to the fungus can lead to invasive infections in people with impaired immune system with a mortality rate ranging from 30% to 95% and emerging as one of the most common causes of infection-related deaths. Important predictors of survival from invasive aspergillosis are early diagnosis and immediate start of appropriate antifungal therapy. The diagnosis of invasive aspergillosis can be challenging and require a combination of clinical, radiological and microbiological techniques. Regarding viral outbreaks, testing, contact tracing and quarantining remain the frontline response to such an outbreak as evidenced by COVID-19 pandemic.

Accordingly, there is a need in the art for cost-effective and simple to use methods and assays for detecting the presence of fungal species and viruses in a sample.

### SUMMARY

The present disclosure relates to kits, compositions and methods for detecting fungal species, viruses and/or protein variants in a sample, e.g., a biological sample. For example, but not by way of limitation, the present disclosure provides living yeast biosensors that have been genetically engineered to detect fungal species, viruses and/or protein variants in a sample, e.g., a biological sample.

The present disclosure provides sensor cells and compositions thereof, e.g., cell compositions, for detecting a fungal species in a sample. In certain embodiments, the sensor cell and/or composition thereof can be used to detect a healthcare-associated infection in a sample. In certain embodiments, the sensor cell and/or compositions of the present disclosure can be used to detect aspergillosis in a sample, e.g., by detecting the presence of a species of the Aspergillus genus in a sample, and methods of use thereof.

In certain embodiments, the present disclosure provides sensor cells for detecting a fungal species in a sample. In certain embodiments, the fungal species is selected from A. nidulans, A. fumigatus, A. terreus, A. flavus, A. niger, A. clawztus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T marneffei. P. carinii, P. murina, P. wakefieldiae, C. dubliniensis, C. auris, C. pseudohaemulonii, C. haemuloni, C. duobushaemulonis, C. metapsilosis, C. orthopsilosis or a combination thereof. In certain embodiments, the sensor cell includes a heterologous GPCR that binds to an analyte derived from the fungal species, wherein the analyte is a ligand for the heterologous GPCR. In certain embodiments, the sensor cell further includes a reporter gene, wherein binding of the analyte to the heterologous GPCR triggers an appearance of the reporter and indicates the presence of the fungal species in the sample.

The present disclosure provides methods for detecting the presence of a fungal species in a sample, wherein the fungal species is A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis, C. orthopsilosis or a combination thereof. An example method includes contacting the sample with a sensor cell comprising a heterologous G-protein coupled receptor (GPCR) that binds to an analyte derived from the fungal species, wherein the analyte is a ligand for the heterologous GPCR. In certain embodiments, the method includes binding of the analyte present in the sample to the heterologous GPCR, wherein binding of the analyte to the heterologous GPCR triggers an appearance of a reporter. In certain embodiments, the method can further include detecting the appearance of the reporter, wherein the appearance of the reporter indicates the presence of the species of the fungal species in the sample.

In certain embodiments, the fungal species is A. fumigatus. In certain embodiments, the analyte is a peptide analyte. In certain embodiments, the peptide analyte is an analyte disclosed in Table 2. In certain embodiments, the sensor cell is S. cerevisiae. In certain embodiments, the heterologous GPCR is a Ste2 receptor from a fungal species disclosed herein. In certain embodiments, the heterologous GPCR includes an amino acid sequence that is at least about 80%, at least about 85%, at least about 90%, at least about 95%, at least about 98% or at least about 99% homologous to any one of the GPCR amino acid sequences disclosed in Tables 5, 6 or 8 or a GPCR engineered by directed evolution to bind the analyte. In certain embodiments, the heterologous GPCR includes an amino acid sequence that is at least about 80%, at least about 85%, at least about 90%, at least about 95%, at least about 98% or at least about 99% homologous to any one of the GPCR amino acid sequences disclosed in Table 5. In certain embodiments, the reporter gene encodes a fluorescent protein, a visible light pigment, a redox peptide and/or a metal-chelating peptide. In certain embodiments, the sensor cell further includes a protease. The present disclosure further provides kits for detecting the presence of a fungal species disclosed herein. In certain embodiments, the present disclosure provides a kit for detecting a healthcare-associated infection. An example kit includes one or more sensor cells described herein. In certain embodiments, the kit can further include means for obtaining the sample from a subject, e.g., a swab, and/or a protease.

The present disclosure relates to sensor cells and compositions thereof, e.g., cell compositions, for detecting a virus in a sample and methods of use thereof. In certain embodiments, a sensor cell of the present disclosure includes (a) at least one heterologous G-protein coupled receptor (GPCR) that binds to an analyte derived from the virus, wherein the analyte is a ligand for the heterologous GPCR; and (b) a reporter gene, wherein binding of the analyte to the heterologous GPCR triggers the expression of the reporter gene and indicates the presence of the virus in the sample.

The present disclosure further provides methods for detecting the presence of a virus in a sample. In certain embodiments, a method of the present disclosure can include (a) contacting a sample with a sensor cell comprising a heterologous G-protein coupled receptor (GPCR) that binds to an analyte derived from the virus, wherein the analyte is a ligand for the heterologous GPCR; (b) binding of the analyte present in the sample to the heterologous GPCR, wherein binding of the analyte to the heterologous GPCR results in an appearance of a reporter; and (c) detecting the appearance of the reporter, wherein the appearance of the reporter indicates the presence of the virus in the sample.

In certain embodiments, the virus can be a respiratory virus, a hemorrhagic virus, a gastrointestinal virus, an exanthematous virus, a sexually transmitted virus, a hepatitis virus and/or a neurological virus. In certain embodiments, the virus is a respiratory virus. For example, but not by way of limitation, the respiratory virus can be an influenza virus, a respiratory syncytial virus, a parainfluenza virus, a metapneumovirus, a rhinovirus, a coronavirus, an adenovirus, a bocavirus or a combination thereof.

In certain embodiments, the respiratory virus is a coronavirus. In certain embodiments, the coronavirus is SARS-CoV-2, MERS-CoV or SARS-CoV. In certain embodiments, the coronavirus is SARS-CoV-2. In certain embodiments, the coronavirus is a variant of SARS-CoV-2, e.g., a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, a SARS-CoV-2 omicron variant or a SARS-CoV-2 zeta variant. In certain embodiments, the SARS-CoV-2 variant is B.1.1.7, B.1.351, P.1, B.1.427, B.1.429 and/or B.1.617.2. In certain embodiments, the analyte is derived from a nucleocapsid protein and/or a spike (S) protein of the coronavirus.

In certain embodiments, the virus is a hemorrhagic virus. In certain embodiments, the hemorrhagic virus is an ebolavirus. In certain embodiments, the ebolavirus is selected from the group consisting of Zaire ebolavirus, Sudan ebolavirus, Tat Forest ebolavirus, Bundibugyo ebolavirus, Reston ebolavirus, Bombali ebolavirus and a combination thereof. In certain embodiments, the ebolavirus is the Zaire ebolavirus. In certain embodiments, the analyte is derived from a small secreted glycoprotein of the ebolavirus. In certain embodiments, the analyte is derived from a small secreted glycoprotein of the ebolavirus. In certain embodiments, the analyte is derived from a VP40 matrix protein of the ebolavirus.

In certain embodiments, the analyte is a full-length protein of the virus. In certain embodiments, the analyte is a peptide analyte derived from a protein of the virus. In certain embodiments, the analyte comprises an amino acid sequence that is at least about 80%, at least about 85%, at least about 90%, at least about 95%, at least about 98% or at least about 99% homologous to any one of the sequences shown in Table 3. In certain embodiments, the analyte is a peptide that is cleaved from the protein of the virus, e.g., by a protease. For example, but not by way of limitation, protease Arg-C or trypsin can be used to cleave an analyte from the nucleocapsid protein.

In certain embodiments, the heterologous GPCR is a fungal mating pheromone GPCR. In certain embodiments, the heterologous GPCR is a GPCR, e.g., a fungal mating GPCR, engineered by directed evolution to bind the analyte. In certain embodiments, the heterologous GPCR comprises an amino acid sequence that is at least about 80%, at least about 85%, at least about 90%, at least about 95%, at least about 98% or at least about 99% homologous to any one of the sequences shown in Tables 5, 6 or 8.

The present disclosure further provides kits for detecting one or more viruses in a sample. In certain embodiments, a kit of the present disclosure includes one or more sensor cells disclosed herein. In certain embodiments, the kit further comprises a protease. In certain embodiments, the one or more sensor cells are provided in one or more containers, e.g., cell culture tubes. In certain embodiments, the kit can further include means for obtaining the sample from a subject, e.g., a nasal swab for obtaining a sample from a subject.

The present disclosure provides sensor cells and compositions thereof, e.g., cell compositions, for detecting a polypeptide variant, e.g., a protein variant, in a sample, and methods of use thereof. In certain embodiments, the compositions of the present disclosure can be used to detect a variant of a protein of a virus, e.g., a SARS-CoV-2 variant.

The present disclosure provides a sensor cell for detecting the presence of a polypeptide variant, e.g., a protein variant in a sample. In certain embodiments, the sensor cell for detecting the presence of a polypeptide variant, e.g., a protein variant in a sample. In certain embodiments, the sensor cell includes (a) at least one heterologous G-protein coupled receptor (GPCR) that binds to a variant of a protein (or a fragment thereof) and the wild type protein (or a fragment thereof); (b) a protease that cleaves either the protein variant or the wild type protein only; and (c) a reporter gene, wherein binding of the protein variant or the wild type protein to the heterologous GPCR results in the expression of the reporter gene. In certain embodiments, the sensor cell includes (a) at least one heterologous G-protein coupled receptor (GPCR) that binds to the wild type protein and one or more variants of the protein; (b) a protease that cleaves one or more of the protein variants; and (c) a reporter gene, wherein binding of the wild type protein to the heterologous GPCR triggers the expression of the reporter gene. In certain embodiments, the sensor cell includes (a) at least one heterologous G-protein coupled receptor (GPCR) that binds to a wild type protein and one or more variants of the protein; (b) a protease that cleaves the wild type protein; and (c) a reporter gene, wherein binding of the one or more protein variants to the heterologous GPCR triggers the expression of the reporter gene.

The present disclosure further provides methods for detecting a protein variant in a sample. An exemplary method can include (a) contacting the sample with a first sensor cell expressing a heterologous receptor that binds to a protein variant (or a fragment thereof) and a wild type protein (or a fragment thereof), where binding of the protein to the heterologous receptor results in the expression of a reporter; (b) contacting the sample with a second sensor cell expressing the heterologous receptor that binds to the protein variant and the wild type protein, where binding of the protein to the heterologous receptor triggers an appearance of a reporter, and expressing a protease that specifically cleaves either the protein variant or the wild type protein only; and (c) detecting the appearance of the reporter in the first sensor cell and the second sensor cell. In certain embodiments, the protease cleaves the protein variant, and if the protein variant is present in the sample then the heterologous receptor of the second sensor cell will not be activated by the protein variant. In certain embodiments, the protease cleaves the protein variant, and if the wild type protein is present in the sample then the heterologous receptor of the second sensor cell will be activated by the wild type protein. In certain embodiments, the protease cleaves the wild type protein, and if the wild type protein is present in the sample then the heterologous receptor of the second sensor cell will not be activated by the wild type protein. In certain embodiments, the protease cleaves the wild type protein, and if the protein variant is present in the sample then the heterologous receptor of the second sensor cell will be activated by the protein variant.

In certain embodiments, the protein variant is clinically relevant to an infection, disease and/or disorder. In certain embodiments, the protein variant is a variant of a protein from a virus. In certain embodiments, the virus is SARS-CoV-2. In certain embodiments, the virus is a SARS-CoV-2 variant. In certain embodiments, the SARS-CoV-2 variant is selected from the group consisting of a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, a SARS-CoV-2 omicron variant, a SARS-CoV-2 zeta variant and a combination thereof. In certain embodiments, the SARS-CoV-2 variant is selected from the group consisting of B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron), B.1.526 (iota) and a combination thereof. In certain embodiments, the protein variant is a variant of a spike (S) protein of SARS-CoV-2.

In certain embodiments, the protein variant comprises an amino acid sequence that is at least about 80%, at least about 85%, at least about 90%, at least about 95%, at least about 98% or at least about 99% homologous to any one of the sequences shown in Table 4. In certain embodiments, the sensor cell is S. cerevisiae. In certain embodiments, the heterologous GPCR is a GPCR comprising an amino acid sequence that is at least about 80%, at least about 85%, at least about 90%, at least about 95%, at least about 98% or at least about 99% homologous to any one of the sequences shown in Table 6 or 8 or a GPCR engineered by directed evolution to bind the protein variant. In certain embodiments, the first and/or second heterologous receptor is a GPCR comprising an amino acid sequence that is at least about 80%, at least about 85%, at least about 90%, at least about 95%, at least about 98% or at least about 99% homologous to any one of the sequences shown in Table 6 or 8 or a GPCR engineered by directed evolution to bind the protein variant. In certain embodiments, the first heterologous receptor and second heterologous receptor are the same. In certain embodiments, the protease comprises an amino acid sequence that is at least about 80%, at least about 85%, at least about 90%, at least about 95%, at least about 98% or at least about 99% homologous to any one of the sequences shown in Table 9 or a protease engineered by directed evolution to cleave the protein variant.

In certain embodiments, a composition for detecting a protein variant further includes (a) at least one heterologous G-protein coupled receptor (GPCR) that binds to the protein variant; and (b) a reporter gene, wherein binding of the protein variant to the heterologous GPCR triggers the expression of the reporter gene, wherein the second sensor cell does not express a protease that cleaves the protein variant.

The present disclosure further provides a kit or product for detecting a protein variant in a sample that includes one or more sensor cells described herein. In certain embodiments, the kit or product can include one or more of the sensor cells of the present disclosure, e.g., a sensor cell that includes (a) at least one heterologous G-protein coupled receptor (GPCR) that binds to the protein variant (or a fragment thereof) and the wild type protein (or a fragment thereof); (b) a protease that cleaves either the protein variant or the wild type protein only; and (c) a reporter gene, where binding of the protein variant and the wild type protein to the heterologous GPCR triggers the expression of the reporter gene. In certain embodiments, a kit or product of the present disclosure can further include a second sensor cell that includes (a) at least one heterologous G-protein coupled receptor (GPCR) that binds to the wild type protein and one or more variants of the protein; and (b) a reporter gene, where binding of the one or more protein variants and the wild type protein to the heterologous GPCR triggers the expression of the reporter gene. In certain embodiments, the second sensor cell does not express a protease that cleaves either the protein variant or the wild type protein.

In certain embodiments, the present disclosure provides a genetically-engineered sensor cell for detecting an analyte in a sample that includes (a) a heterologous G-protein coupled receptor (GPCR) that binds to an analyte derived from the fungal species, wherein the analyte is a ligand for the heterologous GPCR; and (b) a reporter gene, wherein binding of the analyte to the heterologous GPCR triggers the expression of the reporter and indicates the presence of the fungal species in the sample, wherein the genetically-engineered sensor cell comprises a mutation and/or deletion in one or more of the following endogenous genes: Mfa1, Mfa2, Mf(alpha)1, Mf(alpha)2, Bar1, Far1, Sst2, Gpr1, Gpa2, Ste2, sjGFP, LEU2 and URA3. In certain embodiments, the genetically-engineered sensor cell further expresses a heterologous protease. In certain embodiments, the heterologous GPCR comprises an amino acid sequence that is at least about 80%, at least about 85%, at least about 90%, at least about 95%, at least about 98% or at least about 99% homologous to any one of the GPCR amino acid sequences disclosed in Tables 5, 6 or 8 or a GPCR engineered by directed evolution to bind the analyte. The present disclosure further provides kits for detecting the presence of an analyte in a sample that includes one or more of these sensor cells. The present disclosure further provides methods for detecting the presence of an analyte in a sample that includes (a) contacting the sample with a sensor cell described herein; (b) binding of the analyte present in the sample to the heterologous GPCR, wherein binding of the analyte to the heterologous GPCR triggers an appearance of a reporter; and (c) detecting the appearance of the reporter, wherein the appearance of the reporter indicates the presence of the analyte in the sample.

In certain embodiments, the reporter comprises a fluorescent protein, a visible light pigment, a redox peptide and/or a metal-chelating peptide. In certain embodiments, the reporter expressed by a sensor cell of the present disclosure is a visible light pigment. In certain embodiments, the reporter expressed by a sensor cell of the present disclosure is lycopene. In certain embodiments, the sensor cell is of claim 104, the sensor cell is genetically engineered to include a copy of the CrtE gene, a copy of CrtB gene, two or three copies of CrtI and at least one copy of a gene encoding FAD. In certain embodiments, the reporter expressed by a sensor cell of the present disclosure is violacein. In certain embodiments, the sensor cell is of claim 104, the sensor cell is genetically engineered to include copies of the VioA, VioB, VioE, VioD and VioC genes. In certain embodiments, the reporter expressed by a sensor cell of the present disclosure is indigoidine. In certain embodiments, the sensor cell is of claim 104, the sensor cell is genetically engineered to include copies of the idgS and sfp genes.

### BRIEF DESCRIPTION OF THE DRAWINGS

FIG. 1A provides a schematic of a living yeast biosensor for detection of A. fumigatus. The yeast biosensor includes a heterologously expressed A. fumigatus pheromone GPCR (AfuSte2) which after binding of the cognate pheromone peptide (AfuPep) activates native intracellular mating pathway and drives the expression of the readout under inducible promoter.

FIG. 1B provides the dose-response activation in technical triplicate of non-optimized (yMJ194) and optimized (yTC453) biosensing strain transformed with pGPD_AfuSte2_tSTE2 plasmid (pDR01) after an 8-hour incubation.

FIG. 2 provides the specificity and cross-reactivity of AfuSte2.

FIG. 3 provides the yMJ194/pDR01 (yDR01) strain activated by synthetic AfuPep (100 nM, 10 μM, 1 μM) and A. fumigatus clinical isolates sterile culture supernatants. B5233 is a clinical isolate of A. fumigatus.

FIGS. 4A-4D show a modular biosensing component integration toolkit using Golden Gate assembly. FIG. 4A shows four different types of biosensing transcriptional units are cloned into cassette plasmids with appropriate 4-bp GGA overlaps to be assembled in order: constitutive GPCR expression unit, peptide-inducible peptide secretion unit, peptide-inducible readout expression unit and constitutive peptide protease expression unit. FIG. 4B shows cassette plasmids being mixed-and-matched using GGA and assembled into acceptor plasmid where constitutive mCherry is cut out in the correctly assembled integration plasmid which helps guide assembly screening. FIG. 4C provides an assembled acceptor plasmid that constitutes complete integration plasmid which contains biosensing components, integration auxotrophic selection marker and yeast locus 500-bp homology regions upstream and downstream from integrating components. FIG. 4D provides the integration plasmid that is linearized and transformed along with Cas9/gRNA plasmid into parent yeast strain for genomic integration of the biosensing components.

FIG. 5 provides a comparison of natural and fine-tuned intracellular signaling pathway response. Finetuning of pheromone intracellular response pathway significantly improved signal-to-noise ratio, but not EC₅₀ for three tested GPCRs (AfuSte2, SsSte2, CpSte2) and mCherry as a readout.

FIGS. 6A-6B show Aspergillus spp peptide cross-reactivity with Aspergillus spp GPCRs. FIG. 6A shows a 14×13 orthogonality matrix was generated by testing the response of Aspergillus spp and S. cerevisiae GPCRs across Aspergillus spp and S. cerevisiae peptide ligands and water. The test concentration was set at 40 μM of a given peptide ligand. FIG. 6B shows the peptide residue conservation among the reported pheromone peptides.

FIG. 7 provides a dose-response curve for AfuPep activation of the two-cell sensor system.

FIG. 8 provides dose-response curves of SsSte2 mutant hits. Activation of SsSte2 leads to production of mCherry, which has been normalized for cell density (mCherry/OD600). Measurement done in triplicate.

FIGS. 9A-9D show that a yeast consortium improves A. fumigatus biosensor sensitivity. FIG. 9A provides a scheme of a single strain positive feedback loop biosensor (left). Single-strain positive feedback loop is auto-active and poorly responsive to AfuPep activation (right). FIG. 9B provides a scheme of a two-membered split biosensor. First member is activated with AfuPep and as a response expresses mCherry and secretes ScPep. Second member is activated by secreted ScPep and expresses ymTurquoise2 as a response. Second member's starting OD (ODi) is kept at 0.15, while first member's ODi is varied. FIG. 9C shows that member ratio determines total response contribution in the consortia. Higher ODi leads to higher fluorescence intensity normalized to total OD. FIG. 9D shows that the higher ODi of the first member shifts EC₅₀ of the second member, but at the expense of lowering its fluorescence intensity. Measurements done after 10-h incubation.

FIGS. 10A-10B show a two-membered split biosensor with additional positive feedback loop in the second member. FIG. 10A provides a scheme of the biosensor. First member is activated with AfuPep and as a response expresses mCherry and secretes ScPep. Second member constitutively expresses protease ScBar1 which degrades ScPep. The second member is activated by secreted ScPep from the first member and from itself and expresses ymTurquoise2. FIG. 10B provides a two-membered split sensor with positive feedback loop in the second member does not lead to stronger EC₅₀ shift compared to simple split sensor in FIG. 9 with the same ODi ratio. Second member's ODi is kept at 0.15, while first member's ODi is 0.012 (left). The second member activated directly with ScPep shows weaker response compared to direct ScPep sensor (right). Measurements done after 10-h incubation.

FIGS. 11A-11C show a single-strain positive feedback loop biosensor which can be activated with non-cleavable mutant peptide. FIG. 11A provides a scheme of a single-strain positive feedback loop biosensor which can be activated with non-cleavable mutant peptide. FIG. 11B provides a dose-response comparison of three strains responsive to CaPep and non-cleavable CaPep mutants CaPep2A and CaPep2A13A. The strains are either direct sensors (no protease), have expressed protease CaBar1 (protease), or have expressed protease CaBar1 and inducible CaPep secretion (protease+CaPep loop). FIG. 11C provides a dose-response of a positive feedback loop strain with CaPep at 1:2 peptide serial dilution for more precise curve shape determination. Vertical ticked lines separate response phases. Measurements done after 10-h incubation.

FIGS. 12A-12B show a two-membered split biosensor with a non-cleavable peptide secreted from the first member and additional positive feedback loop in the second member. FIG. 12A provides a scheme of a two-membered split biosensor with non-cleavable peptide secreted from the first member and additional positive feedback loop in the second member. FIG. 12B shows the dose-response and activation comparison of the first member (mCherry) and the second member (ymTurquoise2) at ODi 0.45:0.15 (3:1 ratio). CaPep2G secreted from the first member did not significantly shift EC₅₀ of the second member. CaPep2G13A secreted from the first member shifted EC₅₀ of the second member for about one order of magnitude. Measurements done after 10-h incubation.

FIGS. 13A-13B provide a two-membered positive feedback split biosensor. FIG. 13A provides a scheme of a two-membered positive feedback split biosensor. FIG. 13B shows the dose-response and activation comparison of the first member (mCherry) and the second member (ymTurquoise2) at ODi 0.15:0.15 (1:1 ratio). The two-membered biosensor was incubated in four different scenarios where no member expressed ScBar1 protease (top left), the first member only expressed ScBar1 (bottom left), the second member only expressed ScBar1 (top right) and both members expressed ScBar1 (bottom right). Measurements done after 10-h incubation.

FIGS. 14A-14E show lycopene as a visible readout in A. fumigatus biosensor. FIG. 14A provides an overview of the lycopene biosynthetic pathway. FIG. 14B provides a time course of lycopene production in A. fumigatus biosensor strains activated with 1.6 μM AfuPep. Ticked line represents lycopene visible threshold. FIG. 14C shows the AfuPep dose response of lycopene strains over time. FIG. 14D provides the yTC590 dose-response wells after 4-h incubation with sixth well from left (50 nM AfuPep) as color detection limit in liquid culture. FIG. 14E provides a comparison of the lycopene strain from Ostrov et al. (yMJ260) and the improved lycopene strain of the present disclosure (yTC590) activated with their cognate peptides CaPep and AfuPep, respectively.

FIG. 15A provides an overview of the biosynthesis of lycopene.

FIG. 15B shows that the activation of the pheromone response initiates CrtI expression. Deleted Far1 prevents cell-cycle arrest and deleted Sst2 prevents signal attenuation.

FIG. 15C shows the improvement of lycopene readout speed (v2.0).

FIG. 15D provides representative photos of cell pellets corresponding to strains in FIG. 15C.

FIGS. 16A-16C provide the A. fumigatus biosensor strains yTC527 and yTC528 activated with 0 (H₂O) or 10 μM synthetic AfuPep (Pep) in SC/Glc medium supplemented with 5% or 10% YPD. Top images show pellets, bottom images show culture suspension. FIG. 16A shows a 1-hour incubation. FIG. 16B shows a 2-hour incubation. FIG. 16C shows a 3-hour incubation.

FIGS. 17A-17C shows the improvement of the lycopene pathway and culturing conditions. FIG. 17A shows that the pheromone-inducible CrtB leads to constitutively orange and non-responsive strain. FIG. 17B shows that the media concentration affects total lycopene intensity in yTC590 activated with 10 μM AfuPep. FIG. 17C. YPD percentage in complete media affects lycopene color visibility.

FIG. 18A provides a crystal structure of the nucleocapsid, illustrating the accessibility of the exemplary target epitope (red).

FIG. 18B provides a crystal structure of the spike protein, illustrating the accessibility of the exemplary target epitope (red). FIG. 18C provides the nucleocapsid protein epitope alignment to fungal GPCRs. FIG. 18D provides the spike protein epitope alignment to fungal GPCRs.

FIG. 19 shows that the activation of a mutant Scheffersomyces stipites GPCR receptor (SsSte2) with Intermediate-1 leads to production of the fluorescent report mCherry, which has been normalized for cell density (mCherry/OD600).

FIG. 20A provides a crystal structure showing a small secreted glycoprotein (sGP) dimer, with each monomer in complex with two antibodies. The target peptide is highlighted in red on each monomer.

FIG. 20B provides a dose response for a Candida parapsilosis (CpSte2) receptor with a sGP target peptide.

FIG. 20C provides a dose response curve for CpSte2 with sGP target peptide.

FIG. 20D provides a crystal structure of the VP40 dimer. The VP40 epitope is highlighted in purple.

FIG. 20E provides the intermediate peptide design of sGP epitopes.

FIG. 21 provides the GPCRs/Biosensors for Ebola virus Zaire sGP and SARS-CoV-2 nucleocapsid protein (NP).

FIG. 22 provides an exemplary prototype for the living yeast biosensor. Step 1. Activation of the dried yeast by adding 500 μl of tap water. Step 2. Adding the analyte to biosensor by placing the nasal swap sample inside the test tube and closing the cap. Next, shaking the test tube. Step 3. Waiting 1 hour for the response to appear. An orange-red coloration of the solution indicates a positive response while a yellow coloration indicates a negative response.

FIG. 23 provides an exemplary lateral flow prototype of the living yeast biosensor.

FIG. 24 shows the activation of AfuSte2 biosensor in the presence of nasal swab solution at 30° C. (left) and room temperature (right).

FIG. 25A shows the activation of the nucleocapsid biosensor (based on SsSte2 mutants). Activation of SsSte2 for 6 hours leads to production of mCherry, which has been normalized for cell density (mCherry/OD600). Measurement done in triplicate.

FIG. 25B shows the activation of the nucleocapsid biosensor (based on SsSte2 mutants). Activation of SsSte2 for 8 hours leads to production of mCherry, which has been normalized for cell density (mCherry/OD600). Measurement done in triplicate.

FIG. 26 provides dose response curves of S. cerevisiae Ste2p with Smt3p-α-factor fusions, and α-pheromone (Scα). Activation of Ste2p leads to production of mCherry, which has been normalized for cell density (mCherry/OD600). Measurement done in triplicate.

FIGS. 27A-27E provide a protease-based live yeast biosensor. FIG. 27A provides an exemplary paper-based dipstick assay with engineered yeast patches responsive to original and variant peptide with red pigment as a readout and either expressing (+ protease) or not expressing (−protease) specific peptide protease for peptide differentiation. FIG. 27B provides engineered yeast having responsive GPCR (ScSte2) to cognate peptide ScPep and having inducible fluorescent protein expression (LexO). Peptide protease (ScBar1) which cleaves cognate ScPep is constitutively secreted. FIG. 27C provides a peptide dose-response effect of the secreted protease. Peptide cleavage by the protease prevent the activation of cognate GPCR up to the protease saturation point which is observed as a shift in EC₅₀. FIG. 27D provides a hypothetical schematic showing how the variant detection biosensor works. FIG. 27E shows the dose-response curve characteristics of the yeast peptide activation for original and variant peptide differentiation. The GPCR response to original and variant peptide without the protease has to show similar activation curve, while in the presence of the protease, only one of the peptides has to be cleaved and its activation EC₅₀ pushed outside of the expected peptide concentration range (gray box).

FIGS. 28A-28B show that CaSte2 and CaBar1 distinguish point mutations in CaPep. FIG. 28A provides the alanine-scan dose-response of CaPep with or without secreted cognate protease CaBar1. FIG. 28B provides the selected point-mutation peptides which activate CaSte2 within similar concentration range and only one of them is cleaved by CaBar1.

FIG. 29 shows that fungal peptide proteases are highly specific for their cognate peptides. Expected shift in EC₅₀ is observed most strongly only when cognate protease is co-expressed.

FIGS. 30A-30D provide the design of protease-based live yeast biosensor. FIG. 30A shows engineered yeast having responsive GPCR to cognate peptide and having inducible readout expression (LexO). Peptide protease which cleaves cognate peptide is constitutively secreted (pGPD). FIG. 30B shows peptide dose-response effect of the secreted protease. Peptide cleavage by the protease prevent the activation of cognate GPCR up to the protease saturation point which is observed as apparent shift in EC₅₀. FIG. 30C shows dose-response curve characteristics of the yeast peptide activation for original and variant peptide differentiation. The GPCR response to original and variant peptide without the protease has to show similar activation curve, while in the presence of the protease, only one of the peptides has to be cleaved and its activation pushed outside of the expected peptide concentration range (gray box). FIG. 30D shows a paper-based dipstick assay with engineered yeast patches responsive to original and variant peptide with orange pigment as an inducible readout and either expressing (+protease) or not expressing (−protease) specific peptide protease for variant differentiation.

FIGS. 31A-31D provide peptide alanine scans show single amino acid differences in GPCR activation and protease cleavage. FIG. 31A shows ScPep alanine-scan dose-response with or without secreted cognate protease ScBar1. FIG. 31B shows Change in activation span and log EC₅₀ of each mutant compared to ScPep (top) and each mutant activation compared to ScBar1 (bottom). FIG. 31C shows CaPep alanine-scan dose-response with or without secreted cognate protease CaBar1. FIG. 31D shows Change in activation span and log EC₅₀ of each mutant compared to CaPep (top) and each mutant activation compared to CaBar1 (bottom).

FIG. 32 shows that CaBar1 protease in biosensor strain with lycopene readout shifts EC₅₀ of CaPep and CaPep13A, but not CaPep2A in liquid culture. Peptides were added to cells after 1-h preincubation, then the measurements and photos were taken after 8-h incubation.

FIG. 33 shows that yeast pellets detect and differentiate between two peptides differing in single amino acid in their sequence. 2-mL cultures of Ca-responsive biosensor strains with lycopene readout and with (+CaBar1) or without (−CaBar1) expressed protease were incubated for one hour before adding peptides at appropriate concentrations. Photos of the representative pelleted cultures were taken after 7-hour incubation.

FIG. 34 shows that fungal peptide proteases are highly specific for their cognate peptides. EC₅₀ shift is observed most strongly only when cognate protease is co-expressed with cognate GPCR. Measurements done in biological triplicate after 8-h incubation.

FIG. 35 shows that protease expression cassette copy number affects the extent of EC₅₀ shift.

FIG. 36 shows that CaBar1 protease in biosensor strain with lycopene readout can effectively shift EC₅₀ of CaPep and CaPep13A. Measurements done in biological triplicate after 24-h incubation.

FIG. 37 shows that CaBar1 protease in biosensor strain with lycopene readout diminishes color onset of CaPep and CaPep13A, but not CaPep2A at the same peptide concentrations in liquid culture. Peptides were added to cells after 1-h incubation, then the measurements and photos were taken after 8-h incubation.

FIG. 38 provides that a SpPep, SoPep and SjPep alignment shows high sequence homology.

FIG. 39 provides peptides containing two cysteine residues. Non-functional peptides (n.f.) do not activate their putative cognate GPCR and similar putative sequences of the peptide under the same peptide name activate their putative cognate GPCR at relatively different strength (stronger or weaker). Strains with GPCR plasmid are responsive to their functional cognate peptides.

FIGS. 40A-40L provide the dose-response of strains expressing different GPCRs (Ste2) to their cognate peptides (Pep). Synthetic peptides were used as is (untreated), oxidized (1.2% DMSO), or reduced (0.36 mM DTT) in FIGS. 40A-40J. Different peptide oxidation states did not significantly affect cognate GPCR activation. In FIG. 40K and FIG. 40L, the response of two point-mutated BcPep and HjPep1 (FIG. 39) to their cognate GPCRs is shown. Diminished response of mutated peptides is sequence specific and not oxidation state-specific.

FIG. 41 provides cyclic voltammograms of natural peptides containing two cysteine residues. Measurements were done with 500 μM peptides in 10 mM KCl aqueous solution at different scan rates (High: 0.2 V/s, Medium: 0.1 V/s, Low: 0.05 V/s). CaPep is negative control which does not have any cysteine residues. All measured peptides showed similar curves and they were not distinguishable.

FIG. 42 provides cyclic voltammograms of 1 mM metal-binding tripeptides and/or 1 mM CuCl2 in aqueous solution of 50 mM Tris/HCl (pH 7.4) and 0.1 M NaClO4 on a glassy carbon disk. 0.3 V/s scan rate.

FIG. 43 provides a schematic representation of the ATCUN binding site.

FIGS. 44A-44F provide metal-binding peptide absorbance scans measured in 50 mM Tris/HCl (pH 7.4) and 0.1 M NaClO4 buffer. FIG. 44A and FIG. 44B show that peptides alone do not show prominent peaks in 400-800 nm range. FIG. 44C and FIG. 44D show that copper alone and copper-peptide complexes show absorbance peaks in 500-700 nm range in both water and SC/Glc media. FIG. 44E and FIG. 44F show that some nickel-peptide complexes show prominent peaks in 400-450 nm range in both water and SC/Glc media. SC/Glc media gives overall higher background absorbance compared to water. Metal alone and metal peptide complexes measured at 1 mM concentration.

FIG. 45A shows yeast culture pH in buffered and unbuffered media. Yeast strain yTC370 inoculated at ODi 0.15 and incubated over time in SC/Glc media, SC/Glc media+50 mM Tris/HCl (pH 7.4), SC/Glc media+0.1 M NaClO4, or SC/Glc media+50 mM Tris/HCl (pH 7.4)+0.1 M NaClO4. Media pH is decreased over incubation time. NaClO4 reduces buffering capacity of Tris/HCl. Tris/HCl maintains culture pH up to eight hours of incubation.

FIG. 45B provides metal-binding peptides.

FIG. 46 provides a schematic of genetically incorporated unnatural redox-active amino acids in eukaryotes.

FIG. 47 provides a schematic of redox-active unnatural amino acids for de novo design of orthogonal tRNA synthetase-tRNA pairs.

### DETAILED DESCRIPTION

The present disclosure relates to kits, compositions and methods for detecting fungal species, viruses and/or protein variants in a sample, e.g., a biological sample. For example, but not by way of limitation, the present disclosure provides living yeast biosensors that have been genetically engineered to detect fungal species, viruses and/or protein variants in a sample, e.g., a biological sample.

For clarity and not by way of limitation, the detailed description is divided into the following subsections:
I. Definitions;
II. Analytes;
III. Sensor cells;
IV. Receptors;
V. Proteases;
VI. Reporter;
VII. Sensor Cell Compositions;
VIII. Methods of use; and
IX. Kits and Products.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s)” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and still up to 1% of a given value. Alternatively, particularly with respect to biological systems or processes, the term can mean within an order of magnitude, within 5-fold, and within 2-fold, of a value.

The terms “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. In certain embodiments, the polypeptide can be endogenous to the cell, or can be exogenous, meaning that they are heterologous, i.e., foreign, to the cell being utilized, such as a receptor expressed by a yeast cell.

The term “protein” as used herein refers to a sequence of amino acids for which the chain length is sufficient to produce the higher levels of tertiary and/or quaternary structure. This is to distinguish from “peptides” that typically do not have such structure. Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

As used herein the terms “amino acid,” “amino acid monomer” or “amino acid residue” refer to organic compounds composed of amine and carboxylic acid functional groups, along with a side-chain specific to each amino acid. In particular, alpha- or ax-amino acid refers to organic compounds in which the amine (—NH₂) is separated from the carboxylic acid (—COOH) by a methylene group (—CH₂), and a side-chain specific to each amino acid connected to this methylene group (—CH₂) which is alpha to the carboxylic acid (—COOH). Different amino acids have different side chains and have distinctive characteristics, such as charge, polarity, aromaticity, reduction potential, hydrophobicity and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a nucleic acid of the disclosure in vitro and/or in vivo, e.g., in a yeast cell. For example, but not by way of limitation, a nucleic acid of the present disclosure can encode a heterologous receptor for detecting an analyte. Such DNA (e.g., cDNA) or RNA (e.g., mRNA) vectors can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., a receptor.

As used herein, the term “recombinant protein” refers generally to peptides and proteins. Such recombinant proteins are “heterologous,” i.e., foreign to the cell being utilized, such as a heterologous receptor, e.g., a heterologous GPCR, expressed by a yeast cell. In certain embodiments, a heterologous receptor is a receptor that is not native to the cell expressing such receptor.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule, e.g., receptor.

As used herein, the terms “conservative amino acid substitutions” and “conservative modifications” refer to amino acid modifications that do not significantly affect or alter the function and/or activity of the presently disclosed proteins comprising the amino acid sequence. Such conservative modifications include amino acid substitutions, additions and deletions. Modifications can be introduced into the proteins of this disclosure by standard techniques known in the art, such as site-directed mutagenesis and PCR-mediated mutagenesis. Amino acids can be classified into groups according to their physicochemical properties such as charge and polarity.

Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, no more than five residues within a specified sequence are altered. Exemplary conservative amino acid substitutions are shown in Table 1 below.

TABLE 1

| Original | Exemplary Conservative Amino |
|---|---|
| Residue | Acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

As used herein, the term “fusion protein” refers to a protein that includes all or a portion of a protein that is linked, e.g., at the N-terminus or C-terminus, to a second protein or a portion of the second protein.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others-thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing fusion protein genetic molecular components with codon ensuring optimized expression in assay systems as will be understood by a skilled person.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide. In certain embodiments, secretion can utilize the classical secretory pathway of yeast. In certain embodiments, secretion can utilize an efflux pump.

The term “binding,” as used herein, refers to the connecting or uniting of two or more components by an interaction, bond, link, force or tie in order to keep two or more components together, which encompasses either direct or indirect binding where, for example, a first component is directly bound to a second component, or one or more intermediate molecules are disposed between the first component and the second component. Exemplary bonds comprise covalent bonds, ionic bonds, van der Waals interactions and other bonds identifiable by a skilled person. In certain embodiments, the binding can be direct, such as a peptide, e.g., peptide analyte, that directly binds to a peptide-binding element of a protein. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component. Examples described herein include, without limitation, binding of an analyte to a receptor. In certain embodiments, “binding” means the binding of an analyte to a receptor, directly or indirectly, which triggers the expression of a reporter that is indicative of the presence of an analyte.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “derived” or “derive” is used herein to mean to obtain from a specified source.

As used herein, the term “receptor” means a molecule that binds to a ligand. A presently disclosed receptor is positioned, either inherently or by association with a membrane protein, at the cell surface exposed to the extracellular environment. In certain embodiments, the receptor is a protein. In certain embodiments, the receptor is a naturally occurring (native) protein or a portion thereof. In certain embodiments, the receptor is a portion of a naturally occurring protein comprised in a fusion protein with one or more heterologous proteins. In certain embodiments, the receptor is a mutated version of a naturally occurring protein. In certain embodiments, the receptor is a synthetic protein. In certain embodiments, the receptor is a partly-synthetic protein. In certain embodiments, the receptor comprises one or more non-protein elements.

The term “selectively activates,” as used herein, refers to the ability of a ligand, e.g., peptide, to activate a receptor, e.g., preferentially interact with, in the presence of other different receptors. For example, but not by way of limitation, “selectively activates” can refer to the ability of an analyte of a type of a virus, e.g., a coronavirus, to activate a receptor in the presence of other types of viruses. In certain embodiments, “selectively activates” can refer to the ability of an analyte of a variant of SARS-CoV-2 to activate a receptor in the presence of other variants of SARS-CoV-2. In certain embodiments, “selectively activates” can refer to the ability of an analyte of a fungal species, e.g., A. fumigatus, to activate a receptor in the presence of other fungal species. In certain embodiments, “selectively activates” refers to the ability of a wild type protein and variants thereof, to activate a receptor, e.g., preferentially interact with, in the presence of other proteins, e.g., non-related proteins. In certain embodiments, “selectively activates” refers to the ability of a protein variant and/or wild type protein, to activate a receptor, e.g., preferentially interact with, in the presence of other receptors. For example, but not by way of limitation, “selectively activates” can refer to the ability of a variant of a protein, e.g., a variant of a coronavirus S protein, to activate a receptor in the presence of other non-related proteins.

The term “selectively cleaves,” as used herein, refers to the ability of a protease to cleave a protein or variant of a protein in the presence of other proteins or other variants of the proteins.

A “protein variant” or “polypeptide variant,” as used herein, refers to a protein or polypeptide that comprise modifications and/or truncations compared to a parent or wild type protein or polypeptide. In certain embodiments, a protein variant can differ from the parent protein or wild type protein by at least one amino acid modification, e.g., from about one to about ten amino acid modifications. In certain embodiments, the sequence of a protein variant sequence has at least about 80%, at least about 90%, at least about 95% or at least about at least about 99% identity to a parent or wild type protein sequence. In certain embodiments, a protein variant can differ from a different variant of the protein by at least one amino acid modification, e.g., from about one to about ten amino acid modifications. In certain embodiments, the sequence of a protein variant sequence has at least about 80%, at least about 90%, at least about 95% or at least about at least about 99% identity to a different variant of the protein.

As used herein the term “subject” refers to any animal (e.g., a mammal), including, but not limited to, humans, non-human primates, rodents, and the like, which is to be recipient of a particular treatment. In certain embodiments, the subject is a human.

The term “fragment thereof,” as used herein, refers to a fragment of a protein or peptide. In certain embodiments, the fragment comprises at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the amino acids of the intact and/or full-length protein or peptide.

### II. Analytes

The present disclosure provides sensor cells for detecting one or more analytes disclosed herein. In certain embodiments, the analyte to be detecting using the sensor cell composition and methods disclosed herein can include proteins, polypeptides (including amino acid polymers) and/or peptides derived from one or more fungal species and/or one or more viruses. In certain embodiments, the analyte to be detecting using the sensor cell composition and methods disclosed herein can include one or more variants of a protein, polypeptide (including amino acid polymers) and/or peptide.

In certain embodiments, analytes to be detected by the presently disclosed sensor cells include, but are not limited to, proteins, polypeptides (including amino acid polymers) and/or peptides derived from one or more fungal species disclosed herein.

In certain embodiments, analytes to be detected by the presently disclosed sensor cells include, but are not limited to, proteins, polypeptides (including amino acid polymers) and/or peptides derived from one or more viruses disclosed herein, e.g., a respiratory virus, a hemorrhagic virus, a gastrointestinal virus, an exanthematous virus, a hepatitis virus and/or a neurological virus.

In certain embodiments, analytes to be detected by the presently disclosed sensor cells include, but are not limited to, variants of a polypeptide. Alternatively or additionally, the presently disclosed sensor cells are for use in detecting a wild type form of a polypeptide. In certain embodiments, the polypeptide can be a protein or a fragment thereof, a peptide or an antibody or a fragment thereof. In certain embodiments, the presently disclosed sensor cells can be used for detecting one or more variants of a protein or a fragment thereof.

In certain embodiments, the analyte detected by a sensor cell described herein is a fragment of a peptide, a polypeptide, a protein or a peptide epitope of a protein. As used herein, the term a “peptide epitope” refers to a sub-region of amino acids within a larger polypeptide or protein. A peptide epitope can be composed of about 3-50 residues that are either continuous within the larger polypeptide or protein, or can also be a group of 3-50 residues that are discontinuous in the primary sequence of the larger polypeptide or protein but that are spatially near in three-dimensional space. In certain embodiments, the recognized peptide epitope can stretch over the complete length of the polypeptide or protein, the peptide epitope can be part of a peptide, the peptide epitope can be part of a full protein and can be released from that protein by proteolytic treatment or can remain part of the protein molecule. As disclosed herein, the presently disclosed sensor cells can detect full-length proteins, e.g., an epitope (e.g., a peptide epitope), of a full-length protein.

In certain embodiments, the receptor binds specifically to the analyte (e.g., agent-specific peptide) under assay conditions or under natural conditions (for example, but not limited to, at room temperature (e.g., 20-25° C.), at or around body temperature (e.g., 30-40° C.), field temperature (e.g., 5-40° C.) or between about 20-40° C.

### A. Fungal Analytes

In certain embodiments, the analyte is a protein, polypeptide (including amino acid polymers) and/or peptide derived from one or more fungal species that causes a healthcare-associated infection, e.g., a fungal infection that occurs in a healthcare setting.

In certain embodiments, the analyte is a protein, polypeptide (including amino acid polymers) and/or peptide derived from one or more fungal species that causes aspergillosis, blastomycosis, coccidiomycosis, pneumocystis or candidiasis.

In certain embodiments, the analyte is a protein, polypeptide (including amino acid polymers) and/or peptide derived from one or more fungal species selected from A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei, P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, the analyte is a protein, polypeptide (including amino acid polymers) and/or peptide derived from one or more species of the Aspergillus genus, e.g., A. fumigatus.

In certain embodiments, the analyte is a peptide epitope derived from one or more fungal species disclosed herein, e.g., a protein derived from one or more fungal species disclosed herein. In certain embodiments, the peptide derived from a fungal species disclosed herein can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the peptide has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the peptide has a length of about 5 to about 30 residues. In certain embodiments, the peptide has a length of about 5 to about 15 residues.

In certain embodiments, the peptide derived from one or more fungal species disclosed herein is a fungal mating pheromone, e.g., a peptide specific to a fungal pathogen. For example, but not by way of limitation, the analyte is a fungal mating pheromone of the fungal species to be detected.

In certain embodiments, the peptide analyte to be detected is the fungal mating pheromone of a fungal species selected from A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, the analyte is a fungal mating pheromone of a species of the Aspergillus genus. In certain embodiments, the analyte is the fungal mating pheromone from A. nidulans, A. fumigatus, A. terreus, A. flavus, A. niger, A. clavatus, A. oryzae, A. novofumigatus, A. lentulus, A. viridinutans, A. udagawae and/or N. fischeri. For example, but not by way of limitation, the fungal mating pheromones of species of the Aspergillus genus are provided in Table 2.

In certain embodiments, the peptide derived from a species of the Aspergillus genus comprises an amino acid sequence that is at least about 80%, at least about 85%, at least about 90%, at least about 95%, at least about 98% or at least about 99% homologous to any one of the sequences shown in Table 2, e.g., for detecting that species in a sample. In certain embodiments, the peptide derived from a species of the Aspergillus genus comprises an amino acid sequence shown in Table 2, e.g., for detecting that species in a sample.

In certain embodiments, the peptide derived from a species of the Aspergillus genus comprises an amino acid sequence that is at least about 80%, at least about 85%, at least about 90%, at least about 95%, at least about 98% or at least about 99% homologous to SEQ ID NO: 1, SEQ ID NO: 2, SEQ ID NO: 3, SEQ ID NO: 4, SEQ ID NO: 5, SEQ ID NO: 6 and/or SEQ ID NO: 7. In certain embodiments, the peptide derived from a species of the Aspergillus genus comprises an amino acid sequence that is at least about 80%, at least about 85%, at least about 90%, at least about 95%, at least about 98% or at least about 99% homologous to SEQ ID NO: 3. In certain embodiments, the peptide derived from a species of the Aspergillus genus comprises an amino acid sequence set forth in SEQ ID NO: 3, SEQ ID NO: 4, SEQ ID NO: 5, SEQ ID NO: 6, SEQ ID NO: 7, SEQ ID NO: 8 and/or SEQ ID NO: 9. In certain embodiments, the peptide derived from a species of the Aspergillus genus comprises an amino acid sequence set forth in SEQ ID NO: 3.

In certain embodiments, the peptide derived from A. fumigatus is a fungal mating pheromone, e.g., a peptide specific to a fungal pathogen. In certain embodiments, the peptide derived from a species of the Aspergillus genus comprises an amino acid sequence that is at least about 80%, at least about 85%, at least about 90%, at least about 95%, at least about 98% or at least about 99% homologous to SEQ ID NO: 3. In certain embodiments, the peptide derived from A. fumigatus, e.g., for detecting A. fumigatus in a sample, comprises an amino acid sequence set forth in SEQ ID NO: 3.

TABLE 2

| Species | Peptide Analyte |
|---|---|
| A. nidulans | WCRFAGRIC (SEQ ID NO: 1) or |
| | WCRFRGQVC |
| A. fumigatus | WCHLPGQGC (SEQ ID NO: 3) |
| A. terreus | WCWLPGQGC (SEQ ID NO: 4) |
| A. flavus | WCSLPAQGC (SEQ ID NO: 5) |
| A. niger | WCVLPGQPC (SEQ ID NO: 6) |
| A. clavatus | WCELPGQGC (SEQ ID NO: 7) |
| N. fischeri | WCHLPGQGC (SEQ ID NO: 3) |
| A. oryzae | WCALPGQGC (SEQ ID NO: 8) |
| A. novofumigatus | WCHLPGQGC (SEQ ID NO: 3) |
| A. lentulus | WCHLPGQGC (SEQ ID NO: 3) |
| A. viridinutans | WCHLPGQGC (SEQ ID NO: 3) |
| A. udagawae | WCHLPGQGC (SEQ ID NO: 3) |

In certain embodiments, the peptide comprises the amino acid sequence WCX₁LPX₂QGC (SEQ ID NO: 49), where X₁ and X₂ can be any amino acid. In certain embodiments, X₁ is an H, A or E amino acid. In certain embodiments, X₂ is an A or G amino acid.

In certain embodiments, the peptide comprises the amino acid sequence WCX₁LPGQGC (SEQ ID NO: 50), where X₁ can be any amino acid. In certain embodiments, X₁ is an H, A or E amino acid.

### B. Viral Analytes

Analytes to be detected by the presently disclosed sensor cells include, but are not limited to, proteins, polypeptides (including amino acid polymers) and/or peptides derived from one or more viruses disclosed herein. For example, but not by way of limitation, analytes to be detected by the presently disclosed sensor cells include, but are not limited to, proteins, polypeptides (including amino acid polymers) and/or peptides derived from a respiratory virus, a hemorrhagic virus, a gastrointestinal virus, an exanthematous virus, a sexually transmitted virus, a hepatitis virus and/or a neurological virus.

In certain embodiments, the analyte is a protein, polypeptide (including amino acid polymers) and/or peptide derived from one or more respiratory viruses. In certain embodiments, the analyte is a protein, polypeptide (including amino acid polymers) and/or peptide derived from one or more respiratory viruses selected from influenza viruses, respiratory syncytial virus, parainfluenza viruses, metapneumovirus, rhinovirus, coronaviruses, adenoviruses, enteroviruses and/or bocaviruses.

In certain embodiments, the analyte is a protein, polypeptide (including amino acid polymers) and/or peptide derived from one or more hemorrhagic viruses. In certain embodiments, the analyte is a protein, polypeptide (including amino acid polymers) and/or peptide derived from one or more viruses from the viral families, arenaviridae, bunyaviridae, filoviridae and flaviviridae. In certain embodiments, the analyte is a protein, polypeptide (including amino acid polymers) and/or peptide derived from one or more viruses selected from an ebolavirus, Crimean-Congo hemorrhagic fever virus, Marburg virus, Yellow fever virus, the Dengue fever virus, the West Nile viruses and the Zika virus.

In certain embodiments, the analyte is a protein, polypeptide (including amino acid polymers) and/or peptide derived from one or more of gastrointestinal viruses. In certain embodiments, the analyte is a protein, polypeptide (including amino acid polymers) and/or peptide derived from one or more of gastrointestinal viruses selected from noroviruses, rotaviruses and/or astroviruses.

In certain embodiments, the analyte is a protein, polypeptide (including amino acid polymers) and/or peptide derived from one or more of exanthematous viruses. In certain embodiments, the analyte is a protein, polypeptide (including amino acid polymers) and/or peptide derived from one or more of exanthematous viruses selected from rubeola (measles), rubivirus (rubella), herpes (roseola), variola (smallpox), fifth disease and/or chikungunya viruses.

In certain embodiments, the analyte is a protein, polypeptide (including amino acid polymers) and/or peptide derived from one or more of hepatitis viruses. In certain embodiments, the analyte is a protein, polypeptide (including amino acid polymers) and/or peptide derived from one or more of hepatitis viruses selected from the Hepatitis A virus, Hepatitis B virus, Hepatitis C virus, Hepatitis D virus and/or Hepatitis E virus.

In certain embodiments, the analyte is a protein, polypeptide (including amino acid polymers) and/or peptide derived from one or more of neurological viruses. In certain embodiments, the analyte is a protein, polypeptide (including amino acid polymers) and/or peptide derived from one or more of neurological viruses selected from Polio, Meningitis, encephalitis and/or rabies.

In certain embodiments, the analyte is a protein, polypeptide (including amino acid polymers) and/or peptide derived from a sexually transmitted virus. In certain embodiments, the analyte is a protein, polypeptide (including amino acid polymers) and/or peptide derived from herpes simplex viruses (HSV), papillomaviruses (HPV), human immunodeficiency virus (HIV), hepatitis B virus and cytomegalovirus.

In certain embodiments, the analyte is a protein, polypeptide (including amino acid polymers) and/or peptide derived from one or more coronaviruses. In certain embodiments, the analyte is a protein, polypeptide (including amino acid polymers) and/or peptide derived from one or more coronaviruses selected from HCoV-229E, HCoV-NL63, HCoV—OC43, HCoV—HKU1, MERS-CoV and/or SARS-CoV.

In certain embodiments, the analyte is a protein, polypeptide (including amino acid polymers) and/or peptide derived from SARS-CoV-2 or a variant thereof. In certain embodiments, the analyte is a protein, polypeptide (including amino acid polymers) and/or peptide derived from a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, a SARS-CoV-2 omicron variant or a SARS-CoV-2 zeta variant. In certain embodiments, the analyte is a protein, polypeptide (including amino acid polymers) and/or peptide derived from SARS-CoV-2 variants including the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants.

In certain embodiments, the analyte is a protein, polypeptide (including amino acid polymers) and/or peptide derived from an ebolavirus. In certain embodiments, the analyte is a protein, polypeptide (including amino acid polymers) and/or peptide derived from an ebolavirus including the Zaire ebolavirus, Bundibugyo ebolavirus, Sudan ebolavirus, Bombali ebolavirus, Reston ebolavirus and/or Tai Forest ebolavirus. In certain embodiments, the ebolavirus species is the Zaire ebolavirus.

In certain embodiments, the analyte is a peptide epitope derived from one or more viruses disclosed herein. In certain embodiments, the peptide derived from a virus disclosed herein can have a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the peptide has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the peptide has a length of about 5 to about 30 residues. In certain embodiments, the peptide has a length of about 5 to about 15 residues.

In certain embodiments, an analyte of a hemorrhagic virus, e.g., an ebolavirus, can be derived from the small secreted glycoprotein of the hemorrhagic virus, e.g., an ebolavirus. Additional non-limiting examples of analytes for use in the detection of a hemorrhagic virus, e.g., an ebolavirus, include a peptide derived from the proteins VP40, VP35, VP30, NP, GP, VP24 and L. In certain embodiments, an analyte of a hemorrhagic virus, e.g., an ebolavirus, can be derived from the VP40 protein of the hemorrhagic virus, e.g., an ebolavirus. For example, but not by way of limitation, the peptide derived from a hemorrhagic virus, e.g., an ebolavirus, comprises an amino acid sequence that is at least about 80%, at least about 85%, at least about 90%, at least about 95%, at least about 98% or at least about 99% homologous to any one of the sequences shown in Table 3. In certain embodiments, the peptide derived from a hemorrhagic virus, e.g., an ebolavirus, comprises an amino acid sequence shown in Table 3. In certain embodiments, the analyte has the amino acid sequence VNATEDPSSGYY.

In certain embodiments, a peptide of a respiratory virus, e.g., a coronavirus, e.g., a SARS-CoV-2, can be derived from the nucleocapsid protein of the respiratory virus, e.g., a coronavirus, e.g., a SARS-CoV-2. In certain embodiments, a peptide of a respiratory virus, e.g., a coronavirus, e.g., a SARS-CoV-2, can be derived from the S protein of the respiratory virus, e.g., a coronavirus, e.g., a SARS-CoV-2. For example, but not by way of limitation, the peptide derived from a respiratory virus, e.g., a coronavirus, comprises an amino acid sequence that is at least about 80%, at least about 85%, at least about 90%, at least about 95%, at least about 98% or at least about 99% homologous to any one of the sequences shown in Table 3. In certain embodiments, the peptide derived from a coronavirus, e.g., a SARS-CoV-2, comprises an amino acid sequence shown in Table 3. In certain embodiments, the peptide derived from a coronavirus, e.g., a SARS-CoV-2, has the amino acid sequence QIAPGQTGK

TABLE 3

| Virus (Target Protein) | Peptide Analyte |
|---|---|
| Ebolavirus (small secreted glycoprotein; Zaire) | VNATEDPSSGYY |
| Ebolavirus (small secreted glycoprotein; Zaire) | KTTKSWLQK |
| Ebolavirus (small secreted glycoprotein; Zaire) | WLQKIPLQW |
| Ebolavirus (VP40 protein; Zaire) | WTDDTPTGSNGA |
| Ebolavirus (small secreted glycoprotein; Zaire, | GFRSGVPPKVVNYE |
| Sudan and Bundibugyo) | |
| Ebolavirus (small secreted glycoprotein; Zaire, | FHKEGAFFLYDRL |
| Sudan and Bundibugyo) | |
| Ebolavirus (small secreted glycoprotein; Zaire, | KEGAFFLYDRLAST |
| Sudan and Bundibugyo) | |
| Ebolavirus (small secreted glycoprotein; Zaire, | LPTQGPTQQLK |
| Sudan and Bundibugyo) | |
| Ebolavirus (small secreted glycoprotein; Zaire, | QTEPKTSVV |
| Sudan and Bundibugyo) | |
| Ebolavirus (small secreted glycoprotein; Zaire) | SATKRWGFRSGVPPK |
| Ebolavirus (VP40 protein; Zaire) | TGKKVTSKNGQPI |
| SARS-CoV-2 (nucleocapsid protein) | KHWPQIAQFAPSASAFF |
| SARS-CoV-2 (nucleocapsid protein) | WHWPQYGVFAPG |
| SARS-CoV-2 (nucleocapsid protein) | WHWPQIAVFAPS |
| SARS-CoV-2 (nucleocapsid protein) | KHWPQIAQFAPS |
| SARS-CoV-2 (S protein) | YQAGSTPCNGVEGFNCYF |
| SARS-CoV-2 (S protein) | GSTPCNGVEGFNC |
| SARS-CoV-2 (S protein) | GWIPRCGVEGSNC |
| SARS-CoV-2 (S protein) | GWIPCNGVEGFNC |
| SARS-CoV-2 (S protein) | GWIFGTTLDSK |
| SARS-CoV-2 (S protein) | GFQPTNGVGYQPYR |
| SARS-CoV-2 (S protein) | YGFQPTNGVGYQ |
| SARS-CoV-2 (S protein) | QIAPGQTGK |
| SARS-CoV-2 (S protein) | VYSTGSNVFQTR |
| SARS-CoV-2 (S protein) | FDNPVLPENDGVYFASTEK |
| SARS-CoV-2 (S protein) | DLPQGFSALEPLVDLPIGINITR |
| SARS-CoV-2 (S protein) | VYSSANNCTFEYVSQPFLMDLEGK |

### C. Protein Variant Analytes

The presently disclosed sensor cells are for use in determining the presence of variants of a polypeptide in a sample. Alternatively or additionally, the presently disclosed sensor cells are for use in detecting the presence of a wild type form of a polypeptide in the sample. In certain embodiments, the polypeptide can be a protein or a fragment thereof, a peptide or an antibody or a fragment thereof. In certain embodiments, the presently disclosed sensor cells can be used for determining the presence of one or more variants of a protein or a fragment thereof in a sample.

In certain embodiments, the presently disclosed sensor cells can be used for detecting the presence of one or more variants of a protein or a fragment thereof that is associated with, e.g., causes, an infection, disease and/or disorder in a subject.

In certain embodiments, the variant is variant of an antibody or fragment thereof. In certain embodiments, the variant is a variant of an antibody or fragment thereof for treating an infection, disease and/or disorder in a subject.

In certain embodiments, the variant described herein is a fragment of a peptide, a polypeptide, a protein or a peptide epitope of a protein. In certain embodiments, the peptide, polypeptide or protein fragment can have a length of 3 residues or more, a length of 4 residues or more, a length of 5 residues or more, 6 residues or more, 7 residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the peptide has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the peptide has a length of about 5 to about 30 residues. In certain embodiments, the peptide has a length of about 5 to about 15 residues.

In certain embodiments, the variant is a variant of a protein or fragment thereof from a respiratory virus. In certain embodiments, the variant is a variant of a protein or fragment thereof of one or more coronaviruses. In certain embodiments, the variant is a protein or fragment thereof of one or more coronaviruses selected from HCoV-229E, HCoV-NL63, HCoV—OC43, HCoV—HKU1, MERS-CoV and/or SARS-CoV.

In certain embodiments, the variant protein is a variant of a structural protein of a coronavirus, e.g., SARS-CoV-2. In certain embodiments, the variant protein is a variant of the nucleocapsid protein of a coronavirus, e.g., SARS-CoV-2. In certain embodiments, the variant protein is a variant of a M protein of a coronavirus, e.g., SARS-CoV-2. In certain embodiments, the variant protein is a variant of the E protein of a coronavirus, e.g., SARS-CoV-2.

In certain embodiments, the variant is a protein or fragment thereof derived from SARS-CoV-2 or a variant thereof. In certain embodiments, the SARS-CoV-2 variant can be an alpha variant, a beta variant, a delta variant, a gamma variant, an epsilon variant, a kappa variant, an iota variant, an eta variant, a lambda variant, a mu variant, a zeta variant or an omicron variant. In certain embodiments, the variant is a protein or fragment thereof of a SARS-CoV-2 variant including the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants.

In certain embodiments, the variant protein is a variant of a structural protein of SARS-CoV-2. In certain embodiments, the variant protein is a variant of a nucleocapsid protein of SARS-CoV-2. In certain embodiments, the variant protein is a variant of a M protein of SARS-CoV-2. In certain embodiments, the variant protein is a variant of an E protein of SARS-CoV-2. In certain embodiments, the variant protein is a S protein variant or fragment thereof of SARS-CoV-2. In certain embodiments, the variant protein is a S protein variant or fragment thereof of a SARS-CoV-2 variant including the 0.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BM. (omicron) and/or B.1.526 (iota) variants. In certain embodiments, the genetically-engineered cells and methods of the present disclosure can be used to detect SARS-CoV-2 variants associated with one or more substitutions and/or deletions of the amino acids provided in Table 4 and/or associated with one or more of the amino acid mutations provided in Table 4.

TABLE 4

| Amino Acid | Mutation(s) |
|---|---|
| L18 | L18F, L18P |
| H69 | H69- |
| V70 | V70- |
| Y144 | Y144- |
| Y453 | Y453F |
| K417 | K417N |
| S477 | S477N |
| T478 | T478K |
| E484 | E484K, E484Q |
| N501 | N501Y, N501T, N501N |
| D614 | D614G |
| H655 | H655Y |
| Q677 | Q677P |
| P681 | P681H |
| I692 | I692V |
| M1229 | M1229I |
| S3675 | S3675- |
| S3676 | S3676- |
| S3677 | S3677- |

### III. Sensor Cells

The present disclosure provides sensor cells for detecting an analyte disclosed herein. Non-limiting examples of analytes are provided in Section II.

The sensor cells described herein can be genetically engineered cells. In certain embodiments, a sensor cell of the present disclosure can be engineered to comprise one or more components as disclosed herein. As used herein, the term “engineered” means that one or more components are introduced into a sensor cell or its parental cell. For example, but not by way of limitation, a sensor cell disclosed herein can be genetically engineered using a method selected from the group consisting of recombinant DNA techniques (e.g., Reiterative Recombination and CRISPR), natural genetic events, conjugation and a combination thereof.

In certain embodiments, the sensor cell can be a prokaryotic cell or a eukaryotic cell, e.g., a mammalian cell, a plant cell, a bacterial cell or a fungal cell. For example, but not by way of limitation, the sensor cell can be a mammalian cell, e.g., a genetically engineered mammalian cell. In certain embodiments, the sensor cell can be a plant cell, e.g., a genetically engineered plant cell. In certain embodiments, the sensor cell can be a bacterial cell, e.g., a genetically engineered bacterial cell. In certain embodiments, the sensor cell can be a fungal cell, e.g., a genetically engineered fungal cell.

Any fungal strain can be used in the present disclosure. In certain embodiments, the genetically engineered sensor cell of the present disclosure is a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate and combinations thereof.

In certain embodiments, the fungal cell is a yeast cell. For example, but not by way of limitation, the yeast cell can be Saccharomyces cerevisiae, Pichia pastoris or Schizosaccharomyces pombe. In certain embodiments, the sensor cell is Saccharomyces cerevisiae.

In certain embodiments, the sensor cell of the present disclosure is a bacterial cell. Non-limiting examples of bacteria include Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus lichenmformis, Bacillus amyloliquefaciens, Lactococcus lactis, Lactobacillus plantarum, Lactobacillus casei, Lactobacillus reuteri, Lactobacillus gasseri and Escherichia coli. In certain embodiments, the bacteria cell is Escherichia coli. In certain embodiments, the sensor cell of the present disclosure is not a bacterial cell.

In certain embodiments, the sensor cell of the present disclosure is a mammalian cell. Non-limiting examples of mammalian cells include monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells' and myeloma cell lines such as Y0, NS0 and Sp2/0. In certain embodiments, the sensor cell of the present disclosure is not a mammalian cell.

In certain embodiments, nucleic acids of the present disclosure encoding one or more of the GPCRs, reporters, proteases and/or secretable ligands disclosed herein can be introduced into cells, e.g., yeast cells, using vectors, such as plasmid vectors and cell transformation techniques such as electroporation, heat shock, lithium acetate (Li-acetate) and others known to those skilled in the art and described herein. In certain embodiments, the genetic molecular components are introduced into the cell to persist as a plasmid or integrate into the genome. For example, but not by way of limitation, the nucleic acid can be incorporated into the genome of the genetically-engineered cell. In certain embodiments, the cells can be engineered to chromosomally integrate a polynucleotide of one or more genetic molecular components described herein, using methods identifiable to skilled persons upon reading the present disclosure. In certain embodiments, a nucleic acid encoding a secretable ligand, a GPCR, a protease and/or a reporter can be inserted into the genome of a genetically engineered cell using homologous recombination. In certain embodiments, a nucleic acid encoding a secretable ligand, a GPCR, a protease and/or a reporter of the present disclosure can be inserted into the genome of a genetically engineered cell using a Clustered regularly-interspaced short palindromic repeats (CRISPR)/Cas, e.g., CRISPR/Cas9 system.

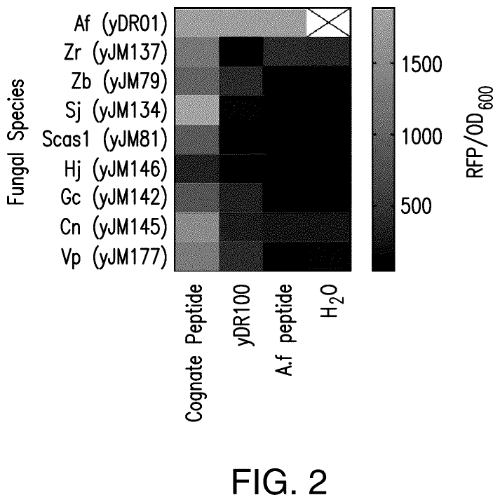

In certain embodiments, a nucleic acid encoding one or more GPCRs, reporters, proteases and/or secretable ligands disclosed herein can be introduced into cells is introduced into the yeast cell either as a construct or a plasmid. In certain embodiments, a nucleic acid can comprise one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid encoding an secretable ligand, a GPCR, a protease and/or a reporter is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pTef1, pPgk1, pCyc1, p., pKex1, pTdh3, pTpi1, pPyk1, pRPL18B, pRev1 and pHxt7 and inducible promoters pGal1, pCup1, pMet15, pFig1, pFus1, GAP, PGCW14 and variants thereof. Additional non-limiting examples of promoters include pTdh3, pCcw12, pPgk1, pTef2, pHhf1, pHtb2, pAld6, pPab1, pRet2, pRnr2, pOpo6, pRad27, pPsp2, pMFa1 and pMFα1 and disclosed in Lee et al. ACS Synth. Biol. 4(9):975-986 (2015)(see FIG. 2 of Lee et al.), the contents of which are disclosed herein in its entirety. In certain embodiments, a variant of Tef1 is scTef1. In certain embodiments, a nucleic acid can include a constitutively active promoter, e.g., pTdh3. In certain embodiments, a nucleic acid can include an inducible promoter, e.g., pFus1 or pFig1. In certain embodiments, a nucleic acid can include a constitutively active promoter, e.g., pAdh1 or LAC4p. In certain embodiments, a nucleic acid can include a constitutively active promoter, e.g., pCyc1. In certain embodiments, a nucleic acid can include a constitutively active promoter, e.g., pRPL18B. In certain embodiments, a nucleic acid can include a constitutively active promoter, e.g., pRev1.

In certain embodiments, a nucleic acid encoding one or more GPCRs, reporters, proteases and/or secretable ligands disclosed herein can further include a transcription factor for regulation expression of the molecule encoded by the nucleic acid. Alternatively and/or additionally, a second nucleic or an additional nucleic acid can be introduced into the cells to express a transcription factor for regulation expression of the GPCRs, reporters, proteases and/or secretable ligands encoded by the nucleic acid. Non-limiting examples of such transcription factors include Abf1p, Aca1p, Ace2p, Adr1p, Aft1p, Aft2p, Arg80p, Arg81p, Arr1p, Ash1p, Azf1p, Bas1p, Cad1p, Cat8p, Cbf1p, Cha4p, Cha4p, Cin5p, Com2p, Crz1p, Cst6p, Cup2p, Dal80p, Dal81p, Dal82p, Ecm22p, Fkh1p, Fkh2p, Flo8p, Fzf1p, Gal4p, Gat1p, Gcn4p, Gcr1p, Gis1p, Gln3p, Gon3p, Gsm1p, Gzf3p, Haa1p, Hac1p, Hap1p, Hap2p, Hap3p, Hap4p, Hap5p, Hcm1p, Hot1p, Hsf1p, Ime1p, Ino2p, Ino4p, Ino4p, Ixr1p, Kar4p, Leu3p, Lys14p, Mac1p, Mal63p, Mbp1p, Mcm1p, Met31p, Met32p, Met4p, Mig1p, Mig2p, Mig3p, Mot2p, Mot3p, Msn2p, Msn4p, Mss11p, Ndt80p, Nrg1p, Nrg2p, Oaf1p, Pdr1p, Pdr3p, Pdr8p, Pho2p, Pho4p, Pip2p, Ppr1p, Put3p, Rap1p, Rcs1p, Rds1p, Reb1p, Rfx1p, Rgt1p, Rim101p, Rlm1p, Rme1p, Rof1p, Rox1p, Rph1p, Rpn4p, Rtg1p, Rtg3p, Sfl1p, Sip4p, Skn7p, Sko1p, Smp1p, Stb4p, Stb5p, Stb5p, Ste12p, Stp1p, Stp2p, Sum1p, Swi4p, Swi5p, Tda9p, Tea1p, Tec1p, Tye7p, Uga3p, Ume6p, Upc2p, Usv1p, War1p, Xbp1p, YER130c, YFL052w, YHR177w, YJL103C, YML081w, YPL230w, Yap1p, Yap3p, Yap5p, Yrr1p, Zap1p and Znf1p. In certain embodiments, a nucleic acid introduced into a genetically-engineered cell of the present disclosure includes one or more DNA binding domains for a transcription factor. In certain embodiments, the DNA binding domain is a zinc finger DNA binding domain. In certain embodiments, the zinc finger DNA binding domain is ZF43-8. In certain embodiments, the transcription factor comprises one or more domains from different proteins. For example, but not by way of limitation, a transcription factor for use in the present disclosure can include an inducer binding domain, e.g., a β-estradiol binding domain, e.g., derived from the human estrogen receptor, and/or a transcription activation domain, e.g., derived from VP64.

In certain embodiments, a nucleic acid encoding one or more GPCRs, reporters, proteases and/or secretable ligands disclosed herein can be inserted into the genome of the cell, e.g., yeast cell. For example, but not by way of limitation, one or more nucleic acids encoding a molecule of the present disclosure, e.g., peptide and/or protein, can be inserted into the Ste2, Ste3 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, one or more endogenous genes of the genetically-engineered cells can be knocked out and/or mutated, e.g., knocked out by a genetic engineering system. Alternatively or additionally, one or more endogenous genes of the genetically-engineered cells can be replaced with a homolog from a different species. In certain embodiments, a genetically-engineered cell can be modified to include multiple copies of an endogenous gene to increase expression of the gene. Various genetic engineering systems known in the art can be used. Non-limiting examples of such systems include the CRISPR/Cas system, the zinc-finger nuclease (ZFN) system, the transcription activator-like effector nuclease (TALEN) system, use of yeast endogenous homologous recombination and the use of interfering RNAs.

In certain embodiments, one or more genes involved in the pheromone sensing pathway can be knocked out, deleted and/or mutated. For example, but not by way of limitation, one or more of the following genes can be deleted: Mfa1, Mfa2, Mf(alpha)1, Mf(alpha)2, alpha-factor protease Bar1, cell cycle arrest inducer Far1, negative regulator of Gpa1 Sst2, glucose sensing GPCR Gpr1 and/or associated G protein α-subunit Gpa2. In certain embodiments, the following genes can also be knocked out in a sensor cell of the present disclosure: Ste2, sfGFP, LEU2 and/or URA3. In certain embodiments, Gpa1 and/or Ste12 are deleted. In certain embodiments, the Gpa1 gene can be replaced with the construct denoted as pPGK1_Gpa1_tENO2. In certain embodiments, the Ste12 gene can be replaced with the construct denoted as pRAD27_LexA-Pheromone-Response-Domain_tENO2. In certain embodiments, a sensor cell of the present disclosure can have the following genes knocked out, deleted or mutated: Mfa1, Mfa2, Mf(alpha)1, Mf(alpha)2, Bar1, Far1, Gpa1 Sst2, Gpr1, Gpa2, Ste2, sfGFP, LEU2, URA3, Gpa1 and Ste12. In certain embodiments, one or more genes involved in the pheromone sensing pathway can be expressed under constitutive promoters.

In certain non-limiting embodiments, a CRISPR/Cas9 system is employed to knock out one or more endogenous genes in the genetically engineered cell. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9) and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule and referred to alternatively as chimeric) or modular (comprising more than one, and typically two, separate RNA molecules, such as a crRNA and a tracrRNA, which are usually associated with one another, for instance by duplexing).

In certain embodiments, a sequence homolog of a nucleotide sequence disclosed herein can be a polynucleotide having changes in one or more nucleotide bases that can result in substitution of one or more amino acids, but do not affect the functional properties of the polypeptide or protein encoded by the nucleotide sequence. Homologs can also include polynucleotides having modifications such as deletion, addition or insertion of nucleotides that do not substantially affect the functional properties of the resulting polynucleotide or transcript. Alterations in a polynucleotide that result in the production of a chemically equivalent amino acid at a given site, but do not affect the functional properties of the encoded polypeptide, are well known in the art.

In certain embodiments, a sequence homolog of a GPCR, reporter, protease and/or secretable ligand disclosed herein can be a peptide, polypeptide or protein having changes in one or more amino acids but do not affect the functional properties of the peptide, polypeptide or protein. Alterations in a peptide, polypeptide or protein that do not affect the functional properties of the peptide, polypeptide or protein, are well known in the art, e.g., conservative substitutions. It is therefore understood that the disclosure encompasses more than the specific exemplary polynucleotide or amino acid sequences and includes functional equivalents thereof.

The cells to be used in the present disclosure can be genetically engineered using recombinant techniques known to those of ordinary skill in the art. Production and manipulation of the polynucleotides described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego.

### A. Sensor Cells for Detection of Fungal Species

The present disclosure provides sensor cells for detecting a fungal species disclosed herein. For example, but not by way of limitation, the present disclosure provides sensor cells that detect an analyte derived from a fungal species.

In certain embodiments, the present disclosure provides sensor cells that detect an analyte derived from a fungal species that results in a healthcare-associated infection.

In certain embodiments, the present disclosure provides sensor cells that detect an analyte derived from a fungal species that causes aspergillosis, blastomycosis, coccidioidomycosis, pneumocystis and/or candidiasis.

In certain embodiments, the present disclosure provides sensor cells that detect an analyte derived from a fungal species selected from A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, the present disclosure provides sensor cells that detect an analyte derived from a fungal species of the Aspergillus genus in a sample. In certain embodiments, the present disclosure provides sensor cells that detect an analyte derived from a fungal species selected from A. fumigatus, A. terreus, A. flavus, A. niger, A. clavatus, A. oryzae, A. novofumigatus, A. lentulus, A. viridinutans, A. udagawae and/or N. fischeri. In certain embodiments, the present disclosure provides sensor cells for detecting A. fumigatus, e.g., an analyte derived from A. fumigatus in a sample.

In certain embodiments, the sensor cells described herein can be genetically engineered to express a receptor for detecting an analyte derived from a fungal species. In certain embodiments, the analyte derived from a fungal species specifically binds to the receptor heterologously expressed by the sensor cell. For example, but not by way of limitation, the sensor cells of the present disclosure express a receptor for detecting an analyte derived from a fungal species that results in a healthcare-associated infection. In certain embodiments, the sensor cells of the present disclosure express a receptor for detecting an analyte derived from a fungal species of Aspergillus, e.g., A. fumigatus, in a sample. In certain embodiments, sensors cells of the present disclosure can be further genetically engineered to express a reporter upon activation of the receptor.

### B. Sensor Cells for Detection of Viruses

The present disclosure provides sensor cells for detecting a virus disclosed herein. For example, but not by way of limitation, the present disclosure provides sensor cells for detecting an analyte derived from a virus in a sample.

In certain embodiments, the present disclosure provides sensor cells for detecting a virus selected from a respiratory virus, a hemorrhagic virus, a gastrointestinal virus, an exanthematous virus, a sexually transmitted virus, a hepatitis virus and/or a neurological virus.

In certain embodiments, the virus is a respiratory virus. Non-limiting examples of respiratory viruses to be detected using the sensor cells of the present disclosure are provided herein, e.g., in Section II. For example, but not by way of limitation, the present disclosure provides sensor cells for detecting an analyte derived from a coronavirus, e.g., an analyte derived from SARS-CoV-2 in a sample.

In certain embodiments, the virus is a hemorrhagic virus. Non-limiting examples of hemorrhagic viruses to be detected using the sensor cells of the present disclosure are provided herein, e.g., in Section II. In certain embodiments, the present disclosure provides sensor cells for detecting an analyte derived from an ebolavirus, e.g., an ebolavirus species, in a sample.

In certain embodiments, the virus is a gastrointestinal virus. Non-limiting examples of gastrointestinal viruses to be detected using the sensor cells of the present disclosure are provided herein, e.g., in Section II.

In certain embodiments, the virus is an exanthematous virus. Non-limiting examples of exanthematous viruses to be detected using the sensor cells of the present disclosure are provided herein, e.g., in Section II.

In certain embodiments, the virus is a neurological virus. Non-limiting examples of neurological viruses to be detected using the sensor cells of the present disclosure are provided herein, e.g., in Section II.

In certain embodiments, the virus is a hepatitis virus. Non-limiting examples of hepatitis viruses to be detected using the sensor cells of the present disclosure are provided herein, e.g., in Section II.

In certain embodiments, the sensor cells described herein can be genetically engineered to express a receptor for detecting an analyte derived from a virus disclosed herein. In certain embodiments, the sensor cells described herein can be genetically engineered to express a receptor for detecting an analyte derived from a coronavirus or an ebolavirus. In certain embodiments, the analyte derived from a virus, e.g., a coronavirus or an ebolavirus, specifically binds to the receptor heterologously expressed by the sensor cell. In certain embodiments, sensors cells of the present disclosure can be further genetically engineered to express a reporter upon activation of the receptor

### C. Sensor Cells for Detection of Protein Variants

The present disclosure provides sensor cells for detecting the presence of a variant of a polypeptide in a sample. In certain embodiments, the polypeptide can include a protein or fragment thereof, a peptide or an antibody.

In certain embodiments, the present disclosure provides sensor cells for determining the presence of antibody variants in a sample.

In certain embodiments, the present disclosure provides sensor cells for determining the presence of a variant of a protein of a virus, e.g., a coronavirus, in a sample. In certain embodiments, the present disclosure provides sensor cells for detecting a SARS-CoV-2 variant, e.g., a protein variant derived from a SARS-CoV-2 variant in a sample. In certain embodiments, variants of the S protein of SARS-CoV-2 can be detected using sensor cells of the present disclosure. In certain embodiments, variants of the nucleocapsid protein of SARS-CoV-2 can be detected using sensor cells of the present disclosure.

In certain embodiments, sensor cells of the present disclosure are genetically engineered to express a receptor that binds to a polypeptide and variants of the polypeptide. For example, but not by way of limitation, sensor cells of the present disclosure are genetically engineered to express a receptor that binds to one or more variants of a polypeptide, e.g., a variant of a protein, and the wild type polypeptide, e.g., wild type protein. In certain embodiments, sensor cells of the present disclosure are genetically engineered to express a receptor that binds to two or more variants of a polypeptide, e.g., a protein, and the wild type protein. Alternatively, or additionally, the sensor cells of the present disclosure are genetically engineered to express a receptor that only binds to the wild type protein or binds to the wild type protein and one or more variants of the protein.

In certain embodiments, sensors cells of the present disclosure can be further genetically engineered to express a protease that selectively cleaves a variant of a protein, e.g., selectively cleaves a single variant of a protein in the presence of two or more variants of the protein or the wild type protein. Alternatively, the protease expressed by the sensor cell selectively cleaves the wild type protein, e.g., selectively cleaves the wild type protein in the presence of one or more variants of the protein. In certain embodiments of the present disclosure, a sensor cell can be genetically engineered to express the protease that cleaves the variant protein and express the protease that cleaves the wild type protein.

In certain embodiments, a sensor cell can be genetically engineered to (i) express a first protease that cleaves a first variant of the protein, (ii) express a second protease that cleaves a second variant protein and (iii) express a protease that cleaves the wild type protein. In certain embodiments, a sensor cell can be genetically engineered to (i) express a first protease that cleaves a first variant of the protein and (ii) express a second protease that cleaves a second variant protein. In certain embodiments, a sensor cell can be genetically engineered to (i) express a first protease that cleaves a first variant of the protein and (ii) express a second protease that cleaves the wild type protein. In certain embodiments, a sensor cell can be genetically engineered to (i) express a first protease that cleaves a second variant of the protein and (ii) express a second protease that cleaves the wild type protein.

In certain embodiments, sensors cells of the present disclosure can be further genetically engineered to express a reporter upon activation of the receptor by the polypeptide, e.g., the wild type polypeptide and the polypeptide variant. For example, but not by way of limitation, if the protease that is expressed by the sensor cell specifically cleaves the polypeptide variant to no longer be able to activate the receptor, then the presence of the wild type polypeptide in the sample activates the receptor. Alternatively, if the protease that is expressed by the sensor cell specifically cleaves the wild type polypeptide to no longer be able to activate the receptor, then the presence of the polypeptide variant in the sample activates the receptor.

### IV. Receptor

In certain embodiments, a sensor cell for use in the present disclosure comprises a heterologous receptor. For example, but not by way of limitation, a sensor cell of the present disclosure comprises a nucleic acid that encodes a heterologous receptor. In certain embodiments, a sensor cell can include one or more, two or more, three or more, four or more, five or more or six or more heterologous receptors. In certain embodiments, a sensor cell of the present disclosure includes one heterologous receptor. In certain embodiments, a sensor cell of the present disclosure includes heterologous receptors.

In certain embodiments, the receptor is a protein. In certain embodiments, the receptor is a naturally occurring (native) protein or a portion thereof. In certain embodiments, the receptor is a portion of a naturally occurring protein comprised in a fusion protein with one or more heterologous proteins. In certain embodiments, the receptor is a mutated version of a naturally occurring protein. In certain embodiments, the receptor is a synthetic protein. In certain embodiments, the receptor is a partly-synthetic protein. In certain embodiments, the receptor comprises one or more non-protein elements.

In certain embodiments, the receptor is a G protein-coupled receptor (GPCR). GPCRs, also known as seven-transmembrane domain receptors, 7TM receptors, heptahelical receptors, serpentine receptor and G protein-linked receptors (GPLR), constitute a large protein family of receptors that detect molecules outside the cell and activate internal signal transduction pathways and, ultimately, cellular responses. In certain embodiments, the GPCR for use in the present disclosure can interact with and activate G proteins. When a ligand binds to the GPCR it causes a conformational change in the GPCR, allowing it to act as a guanine nucleotide exchange factor (GEF). The GPCR can then activate an associated G protein by exchanging the GDP bound to the G protein for a GTP. The G protein's ax subunit, together with the bound GTP, can then dissociate from the β and γ subunits to further affect intracellular signaling proteins or target functional proteins directly depending on the ax subunit type (Gαs, Gαi/o, Gαq/11, Gα12/13).

In certain embodiments, the GPCR is a fungal GPCR. In certain embodiments, the GPCR is a fungal phermone GPCR. In certain non-limiting embodiments, a fungal Ste2-type or Ste3-type GPCR derived from one or more fungus is engineered into a cell to serve as a receptor for detecting an analyte disclosed herein. While any peptide-sensing GPCR can be repurposed as a detection element in a cell, fungal pheromone GPCRs have several key advantages for biosensor engineering. First, this type of GPCRs (GPCRs homologous to the S. cerevisiae Ste2) couple robustly to the host/native pheromone pathway, and several have been expressly validated in S. cerevisiae with little to no further modifications. Second, fungal pheromone GPCRs from related fungi recognize different peptides based on the natural evolution of this class of GPCR. Third, fungal pheromone GPCRs are highly specific for their respective peptides, since they must mediate the species-specific mating reaction while preventing interspecies breeding. In addition, fungal pheromone GPCRs can undergo directed evolution to obtain specificity to various analytes.

In certain embodiments, the receptor is a chimeric protein comprising one or more fragment originating from other receptor proteins, or evolved from a non-homologous receptor protein to bind to the analyte (e.g., agent-specific peptide) and interface with a signaling pathway. In certain embodiments, the receptor is a yeast GPCR polypeptide other than a pheromone binding receptor, such as Gpr1 putative sugar binding receptor and the cognate Gα protein Gpa2.

In certain embodiments, a GPCR of the present disclosure is engineered by directed evolution to alter its stability, specificity and/or sensitivity. Hence, a receptor that is activated by a desired analyte can be generated by mutagenesis and selection in the laboratory, as described herein. Non-limiting examples of such engineered GPCRs include mammalian tachykinin receptors, secretin receptors, opioid receptors, and calcitonin receptors.

In certain embodiments, the receptor is expressed on the surface of the sensor cell. In certain embodiments, the receptor is expressed on internal membranes of the sensor cell. In certain embodiments, the receptor is expressed in the cytoplasm of the sensor cell.

In certain embodiments, the sensor cell is engineered to express the receptor as described herein, for example, by the introduction of a nucleic acid encoding the receptor. In certain embodiments, the nucleic acid is operably linked to a promoter element. In certain embodiments, the promoter element is constitutively active. In certain embodiments, the promoter element is inducibly active. Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15 and pFus1. Additional promoters for use in controlling the expression of the receptors are disclosed in Section III and in Lee et al. ACS Synth. Biol. 4(9):975-986 (2015)(see FIG. 2 of Lee et al.), the contents of which are disclosed herein in its entirety.

In certain embodiments, the receptor is a non-protein molecule. In certain embodiments, the receptor is an aptamer or a riboswitch. The receptor can be comprised of a single element or can be comprised of a plurality of elements/subunits.

### A. Receptors for Detecting a Fungal Species

In certain embodiments, a sensor cell can include one or more receptors that bind to one or more analytes derived from a single species, e.g., fungal species. In certain embodiments, a sensor cell can include one or more receptors that bind to one or more analytes derived from a two or more species, e.g., fungal species. For example, but not by way of limitation, a sensor cell can include one or more receptors e.g., two or more, that bind to one or more analytes derived from a two or more species of the same genus, e.g., fungal genus, e.g., Aspergillus. In certain embodiments, a sensor cell can include one or more receptors, e.g., two or more, that bind to one or more analytes derived from two or more species of different genera, e.g., fungal genera.

In certain embodiments, the analyte to be detected with a heterologous receptor is a fungal mating pheromone of the fungal species. In certain embodiments, the heterologous receptor binds to one or more fungal mating pheromones, e.g., two or more, three or more, four or more, five or more, six or more or seven or more fungal mating pheromones. In certain embodiments, the heterologous receptor binds to the fungal mating pheromones of two or more, three or more, four or more, five or more, six or more or seven or more species of the same genus.

In certain embodiments, the heterologous receptor binds to an analyte derived from a fungal species that causes a healthcare-associated infection, e.g., a fungal infection that occurs in a healthcare setting.

In certain embodiments, the heterologous receptor binds to an analyte derived from a fungal species that causes aspergillosis, blastomycosis, coccidiodmycosis, pneumocystis and/or candidiasis. In certain embodiments, the heterologous receptor binds to an analyte derived from a fungal species that causes aspergillosis.

In certain embodiments, the heterologous receptor binds to an analyte derived from a species of the Aspergillus genus. In certain embodiments, the heterologous receptor binds to an analyte derived from Aspergillus nidulans (A. nidulans), Aspergillus fumigatus (A. fumigatus), Aspergillus terreus (A. terreus), Aspergillus flavus (A. flavus), Aspergillus niger (A. niger), Aspergillus clavatus (A. clavatus), Aspergillus oryzae (A. oryzae), Aspergillus novofumigatus (A. novofumigatus), Aspergillus lentulus (A. lentulus), Aspergillus viridinutans (A. viridinutans), Aspergillus udagawae (A. udagawae) and/or Neosartorya fischeri (N. fischeri). In certain embodiments, the heterologous receptor binds to one or more analytes derived from one or more species of the Aspergillus genus. In certain embodiments, the heterologous receptor binds to analytes derived from two or more species of the Aspergillus genus. For example, but not by way of limitation, the heterologous receptor binds analytes derived from A. fischeri, A. fumigatus, A. lentulus, A. novofumigatus, A. udagawae and/or A. viridinutans. In certain embodiments, a heterologous receptor for use the present disclosure specifically binds to one or more peptides provided in Table 2.

In certain embodiments, the heterologous receptor binds to an analyte derived from a species of the Trichoderma genus. Non-limiting examples of species of the Trichoderma genus include Trichoderma citrinoviride (T. citrinoviride), Trichoderma arundinaceum (T. arundinaceum), Trichoderma longibrahiatum (T. longibrahiatum), Trichoderma harzianum (T. harzianum), Trichoderma guizhouense (T. guizhouense), Trichoderma lentifore (T. lentifore), Trichoderma virens (T. virens), Trichoderma asperellum (T. asperellum), Trichoderma gamsii (T. gamsii) and Trichoderma atroviride (T. atroviride).

In certain embodiments, the heterologous receptor binds to an analyte derived from a species of the Blastomyces genus. Non-limiting examples of species of the Blastomyces genus include Blastomyces dermatitidis (B. dermatitidis) and Blastomyces silverae (B. silverae).

In certain embodiments, the heterologous receptor binds to an analyte derived from a species of the Talaromyces genus. A non-limiting example of a species of the Talaromyces genus is Talaromyces marneffei (T. marneffei).

In certain embodiments, the heterologous receptor binds to an analyte derived from a species of the Coccidioides genus. A non-limiting example of a species of the Coccidioides genus is Coccidioides immitis (C. immitis).

In certain embodiments, the heterologous receptor binds to an analyte derived from Pneumocystis carinii (P. carinii), Pneumocystis murina (P. murina) and/or Pneumocystis wakefieldiae (P. wakefieldiae).

In certain embodiments, the heterologous receptor binds to an analyte derived from Candida dubliniensis (C. dubliniensis), Candida auris (C. auris), Candida pseudohaemulonii (C. pseudohaemulonii), Candida haemuloni (C. haemuloni), Candida duobushaemulonis (C. duobushaemulonis), Candida metapsilosis (C. metapsilosis) and/or Candida orthopsilosis (C. orthopsilosis).

In certain embodiments, a sensor cell of the present disclosure comprises a heterologous receptor that binds to an analyte derived from a species of the Aspergillus genus. In certain embodiments, the heterologous receptor is a GPCR that binds to an analyte derived from a species of the Aspergillus genus.

In certain embodiments, a sensor cell of the present disclosure comprises a heterologous receptor that binds to an analyte derived from A. fumigatus. In certain embodiments, the receptor is a GPCR that binds to an analyte derived from A. fumigatus.

In certain embodiments, a sensor cell of the present disclosure comprises a heterologous receptor that binds to an analyte derived from A. nidulans. In certain embodiments, the heterologous receptor is a GPCR that binds to an analyte derived from A. nidulans.

In certain embodiments, a sensor cell of the present disclosure comprises a heterologous receptor that binds to an analyte derived from A. terreus. In certain embodiments, the heterologous receptor is a GPCR that binds to an analyte derived from A. terreus.

In certain embodiments, a sensor cell of the present disclosure comprises a heterologous receptor that binds to an analyte derived from A. flavus. In certain embodiments, the heterologous receptor is a GPCR that binds to an analyte derived from A. flavus.

In certain embodiments, a sensor cell of the present disclosure comprises a heterologous receptor that binds to an analyte derived from A. niger. In certain embodiments, the heterologous receptor is a GPCR that binds to an analyte derived from A. niger.

In certain embodiments, a sensor cell of the present disclosure comprises a heterologous receptor that binds to an analyte derived from A. clavatus. In certain embodiments, the heterologous receptor is a GPCR that binds to an analyte derived from A. clavatus.

In certain embodiments, a sensor cell of the present disclosure comprises a heterologous receptor that binds to an analyte derived from A. oryae. In certain embodiments, the heterologous receptor is a GPCR that binds to an analyte derived from A. oryae.

In certain embodiments, a sensor cell of the present disclosure comprises a heterologous receptor that binds to an analyte derived from A. novofumigatus. In certain embodiments, the heterologous receptor is a GPCR that binds to an analyte derived from A. novofumigatus.

In certain embodiments, a sensor cell of the present disclosure comprises a heterologous receptor that binds to an analyte derived from A. lentulus. In certain embodiments, the heterologous receptor is a GPCR that binds to an analyte derived from A. lentulus.

In certain embodiments, a sensor cell of the present disclosure comprises a heterologous receptor that binds to an analyte derived from A. viridinutans. In certain embodiments, the heterologous receptor is a GPCR that binds to an analyte derived from A. viridinutans.

In certain embodiments, a sensor cell of the present disclosure comprises a heterologous receptor that binds to an analyte derived from A. udagawae. In certain embodiments, the heterologous receptor is a GPCR that binds to an analyte derived from A. udagawae.

In certain embodiments, a sensor cell of the present disclosure comprises a heterologous receptor that binds to an analyte derived from N. fischeri. In certain embodiments, the heterologous receptor is a GPCR that binds to an analyte derived from N. fischeri.

In certain embodiments, a sensor cell of the present disclosure comprises a heterologous receptor that binds to an analyte derived from a species of the Trichoderma genus, e.g., T. citrinoviride, T. arundinaceum, T. longibrahiatum, T. harzianum, T. guizhouense, T. lentifore, T. virens, T. asperellum, T. gamsii and/or T. atroviride. In certain embodiments, the heterologous receptor is a GPCR that binds to an analyte derived from a species of the Trichoderma genus, e.g., T. citrinoviride, T. arundinaceum, T. longibrahiatum, T. harzianum, T. guizhouense, T. lentifore, T. virens, T. asperellum, T. gamsii and/or T. atroviride.

In certain embodiments, a sensor cell of the present disclosure comprises a heterologous receptor that binds to an analyte derived from a species of the Blastomyces genus, e.g., B. dermatitidis and/or B. silverae. In certain embodiments, the heterologous receptor is a GPCR that binds to an analyte derived from a species of the Blastomyces genus, e.g., B. dermatitidis and B. silverae.

In certain embodiments, a sensor cell of the present disclosure comprises a heterologous receptor that binds to an analyte derived from a species of the Coccidioides genus, e.g., C. immitis. In certain embodiments, the heterologous receptor is a GPCR that binds to an analyte derived from a species of the Coccidioides genus, e.g., C. immitis.

In certain embodiments, a sensor cell of the present disclosure comprises a heterologous receptor that binds to an analyte derived from a species of the Talaromyces genus, e.g., T. marneffei. In certain embodiments, the heterologous receptor is a GPCR that binds to an analyte derived from a species of the Talaromyces genus, e.g., T. marneffei.

In certain embodiments, a sensor cell of the present disclosure comprises a heterologous receptor that binds to an analyte derived from P. carinii, P. murina and/or P. wakefieldiae. In certain embodiments, the heterologous receptor is a GPCR that binds to an analyte derived from P. carinii, P. murina and/or P. wakefieldiae.

In certain embodiments, a sensor cell of the present disclosure comprises a heterologous receptor that binds to an analyte derived from C. dubliniensis, C. auris, C. pseudohaemulonii, C. haemuloni, C. duobushaemulonis, C. metapsilosis and/or C. orthopsilosis. In certain embodiments, the heterologous receptor is a GPCR that binds to an analyte derived from C. dubliniensis, C. auris, C. pseudohaemulonii, C. haemuloni, C. duobushaemulonis, C. metapsilosis and/or C. orthopsilosis.

In certain embodiments, the heterologous receptors for use in the present disclosure are identified by searching protein and genomic databases (e.g., NCBI, UniProt) for proteins and/or genes with homology (structural or sequence homology) to a Ste2 receptor of a species of the Aspergillus genus, e.g., a Ste2 receptor of A. nidulans, A. fumigatus, A. terreus, A. flavus, A. niger, A. clavatus, A. oryae, A. novofumigatus, A. lentulus, A. viridinutans, A. udagawae and/or N. fischeri. In certain embodiments, receptors for use in the present disclosure have homology to a Ste2 receptor of a species of the Aspergillus genus, e.g., at least about 50%, about 55%, about 60%, about 65%, about 70%, about 75%, about 80%, about 85%, about 90%, about 91%, about 92%, about 93%, about 94%, about 95%, about 96%, about 97%, about 98% or about 99% homology to a Ste2 receptor of a species of the Aspergillus genus. In certain embodiments, heterologous receptors for use in the present disclosure have homology to a Ste2 receptor of A. nidulans, A. fumigatus, A. terreus, A. flavus, A. niger, A. clavatus, A. oryzae, A. novofumigatus, A. lentulus, A. viridinutans, A. udagawae and/or N. fischeri, e.g., at least about 50%, about 55%, about 60%, about 65%, about 70%, about 75%, about 80%, about 85%, about 90%, about 91%, about 92%, about 93%, about 94%, about 95%, about 96%, about 97%, about 98% or about 99% homology to a Ste2 receptor of A. nidulans, A. fumigatus, A. terreus, A. flavus, A. niger, A. clavatus, A. oryzae, A. novofumigatus, A. lentulus, A. viridinutans, A. udagawae and/or N. fischeri.

In certain embodiments, the heterologous receptors for use in the present disclosure are identified by searching protein and genomic databases (e.g., NCBI, UniProt) for proteins and/or genes with homology (structural or sequence homology) to a Ste2 receptor of T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis. In certain embodiments, heterologous receptors for use in the present disclosure have homology to a Ste2 receptor of T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis, e.g., at least about 50%, about 55%, about 60%, about 65%, about 70%, about 75%, about 80%, about 85%, about 90%, about 91%, about 92%, about 93%, about 94%, about 95%, about 96%, about 97%, about 98% or about 99% homology to a Ste2 receptor of T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, a GPCR for use in the present disclosure for detecting a fungal species comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in Table 5, e.g., SEQ ID NOs: 9-44. In certain embodiments, the GPCR for detecting A. fumigatus comprises an amino acid sequence set forth in Table 5, e.g., SEQ ID NOs: 9-44.

In certain embodiments, the heterologous receptors for use in the present disclosure are identified by searching protein and genomic databases (e.g., NCBI, UniProt) for proteins and/or genes with homology (structural or sequence homology) to a Ste2 receptor of A. fumigatus. In certain embodiments, receptors for use in the present disclosure are homologous to a Ste2 receptor of A. fumigatus, e.g., have at least about 50%, about 55%, about 60%, about 65%, about 70%, about 75%, about 80%, about 85%, about 90%, about 91%, about 92%, about 93%, about 94%, about 95%, about 96%, about 97%, about 98% or about 99% homology to a Ste2 receptor of A. fumigatus.

In certain embodiments, the GPCR for detecting A. fumigatus comprises the nucleotide sequence set forth in SEQ ID NO: 45 or 46. In certain embodiments, the GPCR for detecting A. fumigatus comprises a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence of SEQ ID NO: 45 or 46.

In certain embodiments, the GPCR for detecting A. fumigatus comprises the amino acid sequence set forth in SEQ ID NO: 10 or the sequence provided in Example 4. In certain embodiments, the GPCR for detecting A. fumigatus comprises amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence of SEQ ID NO: 10 or the sequence provided in Example 4.

Alternatively or additionally, the GPCR for detecting a fungal species disclosed herein can be a fungal GPCR receptor, e.g., a fungal Ste2-type or Ste3-type GPCR from Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for detecting a fungal species comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR receptor of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for detecting a fungal species comprises a GPCR receptor of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate that is engineered by directed evolution to bind the analyte derived from the fungal species.

In certain embodiments, a GPCR for detecting a fungal species in the present disclosure comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of a GPCR disclosed herein, e.g., an amino acid sequence set forth in Table 8. In certain embodiments, a GPCR for detecting a fungal species comprises an amino acid sequence of a GPCR disclosed herein, e.g., an amino acid sequence set forth in Table 8.

In certain embodiments, a GPCR for detecting a species of the Aspergillus genus, e.g., A. fumigatus, comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of a GPCR disclosed herein, e.g., an amino acid sequence set forth in Table 8. In certain embodiments, a GPCR for detecting a species of the Aspergillus genus, e.g., A. fumigatus, comprises an amino acid sequence of a GPCR disclosed herein, e.g., an amino acid sequence set forth in Table 8.

TABLE 5

| Species | GPCR Amino Acid Sequence |
|---|---|
| A. nidulans | MASNEDPWNQTITFRLEDGTPFDISVDYLDGILQYSIRACVNYAAQLGASVILFVILVLLTRAEKRASCL |
| | FWLNSLALLLNFARLLCDVLEFTGNFVRIYTLISADESRVTASDLATSIVGAIMTALLLTTIEISLVLQV |
| | QVVCSNLRRIYRRALLCVSAVVATATIAIRYSLLAVNIRAILEFSDPTTYNWLESLATVALTISICYFCV |
| | IFVTKLGFAIRLRRKLGLSELGPMKVVFIMGCQTLVIPVIVSITHYVSDVPELQTNVLTIVALSLPLSSI |
| | WAGTTIDKPVTHSNVRNLWQILSFSGYRPKQSTYIATTTTATTNAKQCTHCYSESRLLTEKESGRUNDTS |
| | SKSSSQYGIAVEHDISVRSARRESFDV (SEQ ID NO: 9) |
| A. fumigatus | MGSTFDPFSQSITLRGPAENEITIPIETVDFFYQYCIRICINYGAQLGASIVLFIILLLLTRPEKRRSSV |
| | FVLNITALLLNVSRLICQLIYFTSEFVRLYAYFGNDESRVPPSAYADSILGVVLVTLLIVCIEASLVLQA |
| | QVVCANLRRGYRRILLAVSIVMALVPIIFRFVYMVQNCWHIMHAAETIRLNWLESATNVVITISICFFSA |
| | VFITKLGFAMHLRKRLGMTDFGPMKVIFVMGCQTMAIPAIFSILQYIVSVPELASNIFSLVTISLPLSSI |
| | WAGFTIDQRARDSGMSSSRNNLFQALSFSKEAKTRQSSTVTYASQPSSKSHTLCYAELSPAKQQSDAEAA |
| | YGIAVERDISIHSCRKDGKAPNV (SEQ ID NO: 10) |
| A. terreus | MGSTFDPFSQSITLRGPAENEITIPIETVDFFYQYCIRICINYGAQLGASIVLFIILLLLTRPEKRRSSV |
| | FVLNITALLLNVSRLICQLIYFTSEFVRLYAYFGNDESRVPPSAYADSILGVVLVTLLIVCIEASLVLQA |
| | QVVCANLRRGYRRILLAVSIVMALVPIIFRFVYMVQNCWHIMHAAETIRLNWLESATNVVITISICFFSA |
| | VFITKLGFAMHLRKRLGMTDFGPMKVIFVMGCQTMAIPAIFSILQYIVSVPELASNIFSLVTISLPLSSI |
| | WAGFTIDQRARDSGMSSSRNNLFQALSESKEAKTRQSSTVTYASQPSSKSHTLCYAELSPAKQQSDAEAA |
| | YGIAVERDISIHSCRKDGKAPNV (SEQ ID NO: 11) |
| A. flavus | MTMAIMDSKFDPYSQNLTFHAADGTPFQVPVMTLNDFYQYCIQICINYGAQFGASVIIFIILLLLTRPDK |
| | RASSVFFLNGGALLLNMGRLLCHMIYFTTDFVKAYQYFSSDYSRAPTSAYANSILGVVLTTLLLVCIETS |
| | LVLQVQVVCANLRRRYRTVLLCVSILVALIPVGLRLGYMVENCKTIVQTDTPLSLVWLESATNIVITISI |
| | CFFCSIFIIKLGFAIHQRRRLGVRDFGPMKVIFVMGCQTLTVPALLSILQYAVSVPELNSNIMTLVTISL |
| | PLSSIWAGVSLTRSSSTENSPSRGALWNRLTDSTGTRSNQTSSTDTAVAMTYPSNKSSTVCYADQSSVKR |
| | QYDPEQGHGISVEHDVSVHSCQRL (SEQ ID NO: 12) |
| A. niger | MASTDPFAQNVTFHTGDGTPFEVNVQEIDTFLQYCIQICTNYGAQLGASIVLFVILLLLTRPEKRSSSVE |
| | ILNCAALLSNVGRLFCQILYFTTDFVRVYAYFSGDESRVPTSAYANSVIAVVFYTILVICIEASLVLQVQ |
| | VICANLGRVYRQVLLSLSIVVALIPVGFRMAMMVENAIAIMAAESSIPLIWLQSAENITATISICFFCAV |
| | FVAKLGFAIRQRRRLGVRDFGPMKVIFIMGCQTLVIPATFSIIHYIAAVPALSSNVATLVTISLPLSSIW |
| | AGVALTKVSSGAGTGSGTGSGSSGRNLWNVLSFPVWDSRNTTSTTTSSSTPTVNPVSTCYADNGSSKQCK |
| | QQTNTEEIGITVEHEISVQSEKREKDMV (SEQ ID NO: 13) |
| A. clavatus | MNSTFDPFEQSFTLQRSDGKTENTSLVIADHNMEYMLRLSINYGTQLGASIVLLLILMLLTRPEKRLSSV |
| | FLLNVAALASNIVRLVCQISYESSGFAKTYALLAHDFSRVAQATYASQVMASVFFTLVLVCVEISLVLQV |
| | QVVCSNLRRRYRHLLLGASILVTLIPIGFRLAFMVENCIVIVRSGNMKNYHWLESATNIVATVSICFFCA |
| | IFVVKLGFAIKMRRRLGVKDFGPMKVIFIMGCQTMIIPALFSICQYWVNIPEFSHNLLTLVTISLPLSSI |
| | WAGFTLDSSNNTANSTASRRNLWNVLSSNGTVQDKPNLSACSPVASPNTTCYSDPYSGKPHHQDLEAGEG |
| | ISVEHDISIHSYRKDAHSDV (SEQ ID NO: 14) |
| N. fischeri | MNSTFDPWTQNITLTQSDGTTVISSLALADDYLHYMIRLGINYGAQLGACAVLLLVLLLLTRPEKRVSSV |
| | FVLNVAALLANIIRLGCQLSYFSTGFARMYALLAGDESRVSRGAYAGQVMASVFFTIVFICVEASLVLQV |
| | QVVCSNLRRQYRILLLGASTLAALVPIGVRLTYSVLNCMVIMHAGTMDHLDWLESATNIVTTVSICFFCA |
| | VFVVKLGLAIKMRKRLGVKQFGPMRVIFIMGCQTMTIPAIFAICQYFSRIPEFSHNVLTLVIISLPLSSI |
| | WAGFALVQANSTARSTESRHHLWNILSSDGATRDKPSQCVSSPMTSPTTTCYSEQSTSKPQQDPENGEGI |
| | SVAHDISIHSFRKDAHGDI (SEQ ID NO: 15) |
| A. oryzae | MTMAIMDSKFDPYSQNLTFHAADGTPFQVPVMTLNDFYQYCIQICINYGAQFGASVIIFIILLLLTRPDK |
| | RASSVFFLNGGALLLNMGRLLCHMIYFTTDFVKAYQYESSDYSRAPTSAYANSILGVVLTTLLLVCIETS |
| | LVLQVQVVCANLRRRYRTVLLCVSILVALIPVGLRLGYMVENCKTIVQTDTPLSLVWLESATNIVITISI |
| | CFFCSIFIIKLGFAIHQRRRLGVRDFGPMKVIFVMGCQTLTVPALLSILQYAVSVPELNSNIMTLVTISL |
| | PLSSIWAGVSLTRSSSTENSPSRGALWNRLTDSTGTRSNQTSSTDTAVAMTYPSNKSSTVCYADQSSVKR |
| | QYDPEQGHGISVEHDVSVHSCQRL (SEQ ID NO: 16) |
| A. | MNSTFDPWTQNITLTQSDGTTVISSLALADDYLHYMIRLSINYGAQLGACAVLLLVLLLLTRPEKRISSV |
| novofumigatus | FVLNVTALLANIIRLGCQLSYFSTGFARMYALLAGDESRVSRGAYAGQVMASVSFTIVLICVEASLVLQV |
| | QVVCSNLRRRYRILLLGASTLAALVPIGVRLTYSVLNCMVIMHAGTMDHLDWLESATNIVTTVSICFFCA |
| | VFVIKLGLAIKMRKRLGVKQFGPMRVIFIMGCQTMTIPAIFAICQYFSRIPEFSHNVLTLVIISLPLSSI |
| | WAGFALDQANSTARSTESRHHLWNILSSDGATRDKPTQCVSSPMTSPNTTCYSEQSTSKPQQDPENGEGI |
| | SVAHDISIHSERKGANGIFEQSLHLRNA (SEQ ID NO: 17) |
| A. lentulus | MNSTFDPWTQNITLTQSDGTTVISSLALADDYLHYMIRLSINYGAQLGACAVLLLVLLLLTRPEKRVSSV |
| | FVLNVAALLANIIRLGCQLSYFSTGFARMYALLAGDESRVSRGAYAGQVMASVFFTIVLICVEASLVLQV |
| | QVVCSNLRRQYRILLLGASTLAALVPIGVRLTYSVLNCMVIMHAGTMDHLDWLESATNIVTTVSICFFCA |
| | VFVVKLGLAIKMRKRLGVKQFGPMRVIFIMGCQTMTIPAIFAICQYFSKIPEFSHNVLTLVIISLPLSSI |
| | WAGFALDQANSTARSSESRHHLWNILSSDGATRDKPSQCVSSPMTSPNTTCYSEQSTSKPQQDPENEFGI |
| | SVAHDISIHSERKDAHGHL (SEQ ID NO: 18) |
| A | MNSAFDPWTQNITLTRSDGTTVISSLALADEFLRYMIRLSINYGAQLGACVVLLLVLLLLTRPEKRVSSV |
| viridinutans | FVLNVAALLANIIRLGCQLSYFTTGFARIYALLAGDESRVSRGAYAGQVMASVFFTLVLICVEVSLVLQV |
| | QVVCSNLRRQYRILLLGASTLAALVPIAVRLTYSVQNCMVIMHAGTMDHLDWLESATNIVTTVSVCFFCA |
| | VFVVKLGLAIKLRKRLGVKQFGPMRVIFIMGCQTMTIPAIFAICQYFSEIPEFSHNVLTLVIISLPLSSI |
| | WAGFTLEQANSTARSTESRHNLWNILSSDGAVRDKPGQCISSPLTSPNTTCYSQHSTGKPQQDPENGEGI |
| | AVAHDISIHSYQKDARGDV (SEQ ID NO: 19) |
| A. | MNSTFDPWTQNITLTRSDGTTVISSLALADEFLRYMIRLSINYGAQLGASVVLLLVLMLLTRPEKRVSSV |
| udagawae | FMLNVAALLANIIRLGCQLSYFSTGFAKMYALLAGDFSRVSRGAYAGQVMASVFFTLVLICVEVSLVLQV |
| | QVVCSNLRRQYRILLLTASTFAALVPIAVRLTYSVQNCMVIMHAGTMDHLDWLESATNIVTTVSICFFCA |
| | VFVVKLGLAIKMRKRLGVKQFGPMRVIFIMGCQTMTIPAIFAICQYFSKIPEFSHNVLTLVIISLPLSSI |
| | WAGFALEQANPTARSTESRHNLWNVLSSDGAARDKPSQCISSSLTSPNATCYSEQSTSKSEQDPANREGI |
| | SVAHDISIRSYQKDAHGDV (SEQ ID NO: 20) |
| T. | MAFDPYTQNITILLSPSAPPISIPIPLIDAFNDETASIITNYAAQLGAAITMLLVLVAATPSSRLLRAST |
| citrinoviride | LLHAAALLVCVARTVLLAYFFLTPFSHFYQVWTGDFSAVPAWNYRASIAGTVLSTLLAVVTDAALVHQAW |
| | TMVSLFAPRIKFAVCALSLLITLLAVSFRVAYTVIQCEGIADLAAPRRYAWLIRTTLICNICSIGWFCAL |
| | FNSKLISHLVSNRGVLPSRRAMTPMEVLIMANGILMIVPVIFAILEWHHFINFESGSLTPTSIAIILPLA |
| | SLAAQRIATSSASKHHNHPSSSSARYKASRDSASRTPLNAKPGSLFTTTTCNSTASSSAAQRTRSVDASG |
| | MLAAAAGPARGGDFIDPIDLELRQIDGYAQPNEVHFQQDEGQQFHK (SEQ ID NO: 21) |
| T. | MSFDPYSQNITILTPTPSLVPVPIPIPLIDSENDETVSIVINYGAQLGASITMLLVVLFTTPSSKELRAS |
| arundinaceum | NLLHILALLVCIVRTVLLVFYFLSPFSHFYQVWSGDYSQIPAWNYRTSVAGTVESMLLTIITDAALMNQA |
| | WTMVSLWPSKTKYILSFSSMLVTLLAVSFRVAYTVIQCEGIYDLIAPRRFAWLIRTTLIFNICSICWFCA |
| | LENSKLIIHLITNRGVLPSRRAMNPMEVLIMANGILMIVPVVFAVLEWHHFINFEAGSLTPTSVAIILPL |
| | GSLAAQRMAQSAQDASSNISSYPSSSGAAYHGRYKNTRDSGSRTPLKAGSLFSTTTNSSAAAARSQLTAE |
| | NTTLSPVQDIIDPIDLELRQIDGYLQPKETHVKAESSESRFHK (SEQ ID NO: 22) |
| T. | FNPYTQNITILLSPSAPPISIPIPLIDAFNDETVSIITNYAAQLGAALAMLLVLLAATPPSRLLRPGTPL |
| longibrahiatum | VHALALLVCVVRTVLLIYFFLTPFSHFYQVWTGDFSQIPAWNYRASIAGTVLSTLLTIVTDAALVNQAWT |
| | MVSLFAPRTKRIVCLFSVLVALLAISFRVAYTVIQCESIAALAAPRRFAWLIRATLLLNIGSIGWFCALE |
| | NSKLVSHLVTNRGVLPSRRAMSPMEVLIMANGILMIVPVIFAILEWHHFINFESGSLTPTSIAIILPLSS |
| | LAAQRIATSPSSSSSSS (SEQ ID NO: 23) |
| T. | MAFNPYTQNITIIASPDHPPILIPITLIDTENDETVSIVINYGAQLGASLTMLLVILLTTPSAKFLRASC |
| harzianum | LLHVLALIVCAVRTVFLAFYFLSPFSHFYQVWSGDYSQIPAWNYRTSIAGTILSMFLATIAEGALMYQAW |
| | TMVSLFANKAKYAICFLSLLIILITTAFRVAFTVVQCESILNLETSRKYAWLFKTALIENTCSMAWFCAL |
| | FNSKIVHHLISNRGVLPSRRAMTPMEVLIMANGILMFIPFIFAILEWCHFINFEAGSLTPTSIAIVLPLA |
| | SIAAQRISQAPSTPSSEPSYPSSSYQAQYKSTRDSSRTPLKSGSLESSITNTSSATRSHVGDTTVLAPVR |
| | DVIDPIDLELRQIDGYLQPKEIHSRAEAGESQLHK (SEQ ID NO: 24) |
| T. | MAFNPYTQNITVIVSPDYPPILIPIPLIDTENDETVSIVINYGAQLGASLAMLLVILLTTPSSKELRASC |
| guizhouense | LLHVLALIVCAVRTVFLAFYFLSPFSHFYQVWAGDYSEIPAWNYRTSIAGTILSMFLATIAEGALMYQAW |
| | TMVSLFANKAKYAICELSLLIILITTGFRVAFTVIQCESILSLETPQKYAWLFKTALIENTCSMAWFCAL |
| | FNSKIVHHLISNRGVLPSRRAMTPMEVLIMANGILMFIPFIFAILEWCHFINFEAGSLTPTSIAIVLPLA |
| | SIAAQRISQAPSTPSSEPSYPSSSYQAQYKSTRDSSRTPLKSGSLESTISKTSSATRSHVADTTVLAPVR |
| | DVIDPIDLELREIDGYSQPKEIHSRAEAGESQLHK (SEQ ID NO: 25) |
| T. lentifore | MAFNPYTQNITIIVSPDYPPILIPIPLIDAFNDETVSIVINYGAQLGASLTMLLVILLTTPSSKELRASC |
| | LLHVLALIVCAVRTVFLAFYFLSPFSHFYQVWSGDYSQIPEWNYRTSIAGTILSMFLAVIAEGALMHQAW |
| | TMVSLFANKAKYTICFLSLLIILITTAFRVAFTVVQCESILSLESSRKYAWLFKTALIFNTCSMAWFCAL |
| | FNSKIVHHLISNRGVLPSRRAMTPMEVLIMANGILMFIPFFFAILEWCHFINFEAGSLTPTSIAIVLPLA |
| | SIAAQRISQAPSTPSSEPSYPSSSYQAQYKSTRDSSRTPLKSGSLFSAITNTSSATRSHIADTTVLAPVR |
| | DVIDPIDLELRQIDGYVQPKEIQSRTEASESQLHK (SEQ ID NO: 26) |
| T. virens | AKMAFDPYTQSITIHSPPGPPIKIPIPEIDRENEETASIIINYGAQLGASITMLLVILLTVPTQKFLRAS |
| | NLLHILALLVCVFRTVFLVLYFISPFSHFYQVWSGDYSQIPAWNFRTSIAGAVLPMLLTVTTEAALMHQA |
| | WTMVSLFANKTKYAITFLSLLLTLIAIAFRVAYTVAECKSIIDLVSAEKYAWLIKTTLIWNTCSVGWFCA |
| | LENSKLVWHLVSNRGVLPSRRAMTPMEVLIMANGILMIFPVIFATLEWYHFINFEAGSLTPTTVAIILPL |
| | GTLVAQRLSQTSPAPSTDPSSYPSSSYQARYKATRDSSRTPFKSGSLFSATTNSSAPRSQLVDTTVLAPV |
| | RDVIDPIDLELRQIDGYLQPKEIHVRAEPGEGQFHK (SEQ ID NO: 27) |
| T. | MAFDPYTQNITIAITPNVSIPXPIPVIDIFNDETVNIVLNYGTQLGAAITMLLVVLFTTPPAKAFRTANL |
| asperellum | LHVVGLLVCIVRTVLLALYENSPFAHFYAVWINDYSQVPAWTFRESVAGTVESMLLVIISDVSLINQAWT |
| | MVSLWPSRTKYALCFVSLLISLLSIAARTAFTVIQCEGIYELDPPMQFAWLVHAMVILNICTTLWFCALF |
| | NSKLVIHLITNRGVLPSRRAMSPMEVLVVTNGVLMIIPVIFAFIEFRPVVNFESSSLTPASIALILPLGS |
| | LVAQRLAQTNQNASPNLFSYPSSSGAGGYKNNHSSGSTTPLKPGSLFATSTNSSISAARSKSNSQTITAV |
| | PTRDIVDPIELELRQIDGYLQPQETHVSAELGGSRSHK (SEQ ID NO: 28) |
| T. gamsii | MSFNPYAQNVTIRISPALPALHIPLHFIDSENDETVSIVLNYGTQLGAAIMMLLVVLFTTPPSKFLRTAH |
| | LLHLLGLLVCIVRTVLLALYFESPFAHFYAVWSGDYSGVPSWTYRESVAGTVLSMLLVVVNDMALMNQAW |
| | TMVSLWPTATKFVLCAVSLLFALLSVAARTAFTVIQCKAIYALEPAVQFAWLVHAMVIINICTTLWFCAL |
| | FNSKIIIHLIANRGVLPSRRAMSPMEVLVVTNGALMIIPVIFAFIEFRPILNFESSSLTPASITLILPLG |
| | SLVAQRLAYTNQNAVPSLSGYPSSTGAGGYKNNRSSGSNTPLKAGSLFATSTNNSISAARSHSTSRNTTT |
| | VCARDVVDPIELELRQIDGYLQPQETHISADLGGSQSHK (SEQ ID NO: 29) |
| T. atroviride | MALFDPSTQDVTIHISPSAPPLHIPLRAIDGENDETVSIVLNYGTQLGAAITMLLVLLFTTPPSKFLRTA |
| | HLLHLLGLLVCIVRTVLLALYFESPFAHFYAVWSNDYSAVPAWTYRESVAGTVFSMLLVVVNDVALMNQA |
| | WTMVSLWPTGTKLVLCAVSLLVSLLSVAARTAFTVIQCKAIYDLTPAVQFAWLVHAMVILNICTTVWFCA |
| | LENSKIIIHLITNRGVLPSRRAMSPMEVLVVTNGALMIIPVIFAFIEFRPILNFESSSLTPASITLILPL |
| | GSLVAQRLAQTNQNAIPSLSGYPSSTGAGGYKNNRGSGSGSHTPLKAGSLFATSTNNSVSAARSHSTSRN |
| | TTTVPARDIVDPIELELRQIDGYLQPQETHISAELGGSQSHK (SEQ ID NO: 30) |
| B. | MASSFDPFDQNVVFHQADMTPFNVSIHDLDDFVQYGTRVCINYASQLGASVIAIIMLALLTQSEKRRLPV |
| dermatitidis | FSLNISALIMNFARLLCMTIYFTTGENNPYAFFSGDYSRVPGSAYADSILGIVFTTILVICMETSLVIQT |
| | QVVCATLPEIQRYLLLGVSLVIALLAIGERMGLMVENCIAIMNASNFAPFIWLQSASNITITMSTCFFSA |
| | IFVAKLGYALVTRRRLGMTRFGAMQVMFIMSCQTMVIPAIFSIIQYPFPGYEMNSNLLTLVAVELPLSSL |
| | WAAAATKSSFETLNSGPHQYLWSSERSNSTSSATRHPGSFSQNQATLRSGGSVATTLSPDQLDRLYTDLD |
| | VSVKA (SEQ ID NO: 31) |
| B. silverae | MASSFDPFDQNVVFHQADGTPFNVSIHDLDEFVQYGTRVCINYAAQLGASVIAIIMLALLTRSEKRRLPV |
| | FSLNLAALIMNFARLLCMTIYFTTGFNNPYAFFSGDYSRVPGSAYADSILGIVETTILVICMETSLVIQT |
| | QVVCATLSEIQRYLLLAVSLVIALLAIGERMGLMVENCIAIINASNFGPFIWLQSASNITITISTCFFSA |
| | IFVVKLGYALVTRRRLGMTRFGAMQVMFIMSCQTMVIPAIFSIIQYPFPLYEMNSNLLTLVAIFLPLSSL |
| | WAAAATKSSFETLNSGPHQYLWSSERSNSTSSAPGQHGNFSQNQSTLRSGGSVATTLSPDQLDRLYTDLD |
| | TSAKA (SEQ ID NO: 32) |
| T. marneffei | MSFNPYEQNITLTASDGSAMDIPLPYFDEYVQEGVVSCINYGCQLGATIVIFVLLCLLTRSKKRSSIVFC |
| | LNITALLLNMFRLICQVLLYTGAWFETYAYFTGDISRVTRGNFADSILVSVMDSLLQVVMEASLVTQTNV |
| | VCTNMSRWQKTAILTVSVGLAFSSIGERMAQMGLESVLIVENKPFDEFLWLQKLNTILTTTSIIYFSVVL |
| | ICKLGYTIYLRRQLGVHQFSPMQIIFIMSCQTMIVPAILSILQFSSVNIHHINNDVVTFVVVSLPVTTLW |
| | ASNVLPGRAAVHDAESGPPLWNKLSTPRLNSVGSDQFRRKPASPSNPIAFPSTMNFQGSELAKSVTIGTR |
| | LEGSISP (SEQ ID NO: 33) |
| C. immitis | MVDTSGFGNNFDPFTQPVRLYYPNGTDFHRSLAEIDWILRDNTNQSIAYGCQLGASLTMIIILLLLTPPG |
| | KRRSPVFMLNAAALVLNFGRILCSCIYYTTGFSDVYAMLAPDFSRVSVGAYANTELGAILHALLLICVEI |
| | SLLFQTHVLCSTIRSAYRRLALSFSVVLVLAAIGCRLMQTIENCRATINLKSFASFIWLQSLTNILTTVS |
| | ICYFSTIFVAKLGFAIYARRTLGLTGFGAMQVMFIMSCQSMIVPARRLTHDLAIFSILHHFIDAAEMAPL |
| | VLTLVTLSLPLSSMWAAQAVKNSFEGDNRGPTQQRHLFTQGSSTAQTSMASTLVAKRTSSPDHLDRLYPD |
| | IDAIEIRVDRDFTVDIEKGQ (SEQ ID NO: 34) |
| C. | MNSTIIPPNDSGDIIINYLIPGLNESIEIPFHLLDSFQTDQTKLALIMGITIGSCSMTLIFLISIMYKTN |
| dubliniensis | KLSNIFNNNINIKTIFQWINQMIFNEKINLNKHKQNDSYNNNIIVGKGFYKLFLFYLNSLILLIGIIRSG |
| | CYLNYNLGPLNSISFVFTGWYNESISTSKLSFISSDITNGEKCILYGLVEISLGFQVYIMFKTSNLKIWG |
| | LMASLLSISLGLIVVALQINLTILSHIRFVKATTTTTTTNNNETSTSIVTGGYVINSIWMDLPTILFSIS |
| | INIMTILLIGKLIIAIRIRRYLGLKQFDSFHILLIGESQTLIIPSIILIIHYFYLSKSKDSLLQQISLLL |
| | IILMLPLSSLWAQTANNIHNINSSPSLSFISRHHSSDNNSNINTIVNGNNNHLYNSNSNSNSNSNSNGNG |
| | NGKFSKLNTNGSSPFTLKGDSGSLMLQMTSNSNNGNNGNINAGNVNAGNNESIPVSGIDAQLPPDIEKIL |
| | HDEDNINNYKLLNINNENINDGDIIINDEGMITKQITIKRV (SEQ ID NO: 35) |
| C. auris | MEFTGDIVLKYTLGGEEYSSTFEQLDSSVNRSLELGVVHGIAIACGVLLMVLAWVIIIKKKNPIFVLNQL |
| | TLLLMVIKSSLYLAFLFGPLSSLTYKFTRVLPHDKWHAFHVYIATNVIHTLLIATVEMTLVFQIYIIFKS |
| | PEVRHLGYILTGAASALALTIVALYIHSTVISAVQLKEQLLMHEIKITNSWVNNVPIILFSASLNVVCII |
| | LIAKLALAIKTRRYLGLKQFDGLHILMITSTQTFIVPSVLMIVNYKQSSSYSTLLANISVILVVCNLPLS |
| | SLWAASANNSSTPTSSANTVESRWDSKESDTETIAHELPLIPGKAEKLQLVSPITEKGDTHTMCESHGDQ |
| | DSIDKMLDDIEGAVMTTEFNLNNRTV (SEQ ID NO: 36) |
| C. | MDFSNDIVLNYTVAAEEYQLTFGILDSTVDRTLQLCVVHGMAIGCGALLMLLSWVVIIKKTTPMFILNQL |
| pseudo- | TLMLMIIKSALFLCFLFGPLSSLTYKFTGLFPEERWHAFHIYIATNTIHTLLIATVEATLVFQIYVVEKS |
| haemulonii | PEVRNLGYIMTCAAGALATTIVSLYIHSTVISARQLRQQLMEQDVTPTNSWVNNVPVILFSASLNVVCII |
| | LISKLALAIKTRRYLGLKQFDGLHILIITSTQTFIVPSVLMIVNYRQSTSYSTLLANISVILAVCNLPES |
| | SLWATSANNSPTPTSSPNTVFSRWDSKISDTETLNQDMPLIPGKAEKIQLVSPTTEKGDVQTVCGSDGDQ |
| | DLIDRILDDMDGAVMTTELNLRDRMC (SEQ ID NO: 37) |
| C. | MEFSDNDIVLNYTVGPEIYQLTFGILDNTVDRSLQLCVVHGVAIGCGILLMLLSWVVIIKKRTPMFILNQ |
| haemuloni | LTMVLMIIKSALYLCFLFGPLSSLTYKFTGLLPEERWHAFDIYIATNVIHTLLIATVEATLVFQIHVIFK |
| | SPEVKHLGYVLTAAAGSLAVTIVSLYIHSTVVTAIQLRNQLMEQDVTPTNSWVNNVPVILESASLNVVCI |
| | ILISKLALAIRTRRYLGLKQFDGLHILIITSTQTFIVPSVLMIVNYRQSTSYSTLLANISVILAVCNLPF |
| | SSLWATSANNSTHPTSSPNTVESRWDSKLSDTETLAHEVPLLPGKAEKIQLVSPTTEKGDVQTMCDSNGD |
| | QDSIDRMLDDIDGAIMTTELNIRDKLV (SEQ ID NO: 38) |
| C. | MDFSGDSLLNSTATAEEYQLMFGILDSTVERALQLCVVHGMAIGCGALLMLLSWVVIIKKTTPTFILNQL |
| duobushaem | TLVLMIIKSALYLCFLFGPLSSLTYKFTGLFPEGRWHAFHIYIATNTIHTLLIASVEATLVFQIYVVEKS |
| ulonis | PEVRKLGYFMACAAGALATTIVSLYIYSTVLSARQLRQQLLEHEFTPTNSWINNVPVILFSASLNVVCII |
| | LIAKLALAIKTRRYLGLKQFDGLHILTITTTQTFILPSVLMIVNYKQSSSYSTLLANISVILVVCNLPES |
| | SLWATSANNSPTPTSSPNTVFSRWDSKTSDTETLNQDTPLIPGKAEKIQLVSPTTEKGDVQTVCGSDGDH |
| | DSIDRILDDMDGALMTTELNLRDRMC (SEQ ID NO: 39) |
| C. | MDQIVSNLSSGDIFVTITIPNEEDGTYEVPFYVIDQYHNSKMENAVILGATIGACSMLLIMLIGILFKNF |
| metapsilosis | NKLKKSLLFNLNFAVLLMLIMRSACYINYLMNNLSSISFFFTGIENGESEMSSDAANAFKVILVALIELS |
| | LTYQIYIMFKTPTLKVWGVIASLLAGALGLATIATQIYTTVMSHINFVNGTTGSPSQIVTAWMDMPTILF |
| | SVSVNVLSLFLVCKLGLAIRTRRYLGLKQFDAFHILFIMSTQTMIIPSIILFVHYFDQDDSQTTLVNVSL |
| | LLVVISLPLSSLWAQTANNVRRIDTSPSMSFISREASNRSGQETLHSGAKISKYNTSNTVNSTPGTLKDD |
| | SSFILDRPIEQDKVDTGLPKDLEKFLNNDENEGDDGMIAREITMLKTQHPRQ (SEQ ID NO: 40) |
| C. | MNQIVSKLNSGDIIVTFTISNEEDGTYEVPFYVLDEYHRSKMQNAIVLGATIGACSILLIMLIGILFKKF |
| orthopsilosis | QKLKKSLLENLNFAILLMLIMRSACYINYLMNNLSSIGFFFTGVEDGESEMSSDAANAFKVILVALIETS |
| | LTYQICIMFKTPTLKIWGILASFSAGALGLVTIATQIYTTVMSHRNFVNGTTGSPSQITFAWMDMPTILF |
| | SVSVNVLSLFLVCKLGLAIRTRRYLGLKQFDAFHILFIMSTQTMIIPSIILFVHYFYRDDSQTTLVNISL |
| | LLVVISLPLSSLWAQTANNVRRIDTSPSMSFISREISNRSGNETLHSSAKISKYNTSNTVNSTPGTLKDD |
| | SSFILDRPVSEQKMVDTGLPKDLEKFLNNDENEGDDGMIAREVTMLKTNHTNQ (SEQ ID NO: 41) |
| P. carinii | MVFSPVNQTVLLKNSKGDTIPFLLSDEDEFSLSRAQTSMIFSAQCAMSLLLAIVLILTSKREKRKTLLFF |
| | LNIGGLITVFIRACLQCAYLSGTWVSYSVQFLGEFELLSQKDFYVSIIASCIPIFIILFIELSLLIQIRV |
| | VYATEKKLQIPLTIAFSIIIIVVIIFWILAAVQNSMAVLSQTHFGHSGLWGSPWPYTVARISFAFSIFIG |
| | CMVFIYKLLITIYRRHKMGVKEFGPLQIIFIMSCQTLIIPAILILVDFGVKITGFSSLTQALVVMSLPLS |
| | SLWASSKVENNKNNAAPTYYKDMKSIGDYSIESTPSSFTKPPYMGFRKPSYFSEYSRSPFYEEYFDDNGS |
| | KLIY (SEQ ID NO: 42) |
| P. murina | MAFSPTNQTIFLKNSKGETIPFLLSDEDEFSLSRAQTSMIFSAQCAMSLLLALVLMLTSKREKRKTLLFF |
| | LNIGGLVTVFIRACLQCAYLSGTWVSYSVQFLGEFELLSQKDFYISIIASCIPIFIILFIELSLLIQIRV |
| | VYATDKRLQIPLTIFFSIIIIIVITEWILAAVQNSMAVLSQTHFGHSGVWGAPWPYTVARISFAFSIFIG |
| | CIVFIYKLLITIYRRHKMGVKEFGPIQIIFIMSCQTLIIPAILILIDFGVKITGFSSLTQALVVMSLPLS |
| | SLWASSKVEKNKNNVTPTYYKDMKSIGDYSIESTPSSFTKPSYIGFRKPSYFSEYSKSPFYDEYFDDNGS |
| | KLDILVEKSLNVFPEKK (SEQ ID NO: 43) |
| P. | MGFSPVNQTVLLKNSNGETIPFLLSDEDYFSLSRAQTSMIFSAQCSMSLLLALVLILTSKKEKRKTLLFF |
| wakefieldiae | LNIGGLITCTISRRVSIVVAKRLLYINCHIIVVYATDKRFQIPLTTFFSMIIIAVLTFWILAAVQNSMAV |
| | LSQTHFGHSGLWGSPWPYTAARISFAFSIFIGCIVFIYKLLVTIYRRHKMGVKEFGPLQIIFIMSCQTLI |
| | IPVILILIDFGVKITGFSSLTQALVVMSLPLSSLWASSKVENTKNNVRPTYYNDMKSIGDYSIESTPSSL |
| | TKPLYMGFRKPSYFSEYSKSPFYDDYFDDNGCKLDILVEKSLNVLPEKK (SEQ ID NO: 44) |

In certain embodiments, a GPCR detecting a fungal species comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence set forth in Table 7. In certain embodiments, a GPCR for detecting a fungal species comprises an amino acid sequence that is encoded by a nucleotide sequence of a GPCR disclosed herein, e.g., a nucleotide sequence set forth in Table 7.

In certain embodiments, a GPCR for detecting a species of the Aspergillus genus, e.g., A. fumigatus, comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence of a GPCR disclosed herein, e.g., a nucleotide sequence set forth in Table 7. In certain embodiments, a GPCR for detecting a species of the Aspergillus genus, e.g., A. fumigatus, comprises an amino acid sequence that is encoded by a nucleotide sequence of a GPCR disclosed herein, e.g., a nucleotide sequence set forth in Table 7.

### B. Receptors for Detecting a Virus

In certain embodiments, a sensor cell of the present disclosure comprises a heterologous receptor for binding to an analyte derived from a virus disclosed herein. For example, but not by way of limitation, a sensor cell of the present disclosure comprises a nucleic acid that encodes a heterologous receptor. In certain embodiments, a sensor cell can include one or more, two or more, three or more, four or more, five or more or six or more heterologous receptors. In certain embodiments, a sensor cell includes one heterologous receptor. In certain embodiments, a sensor cell includes two heterologous receptors.

In certain embodiments, a sensor cell can include one or more receptors that bind to one or more analytes derived from a single virus. In certain embodiments, a sensor cell can include one or more receptors that bind to analytes derived from two or more viruses. For example, but not by way of limitation, a sensor cell can include one or more receptors e.g., two or more, that bind to one or more analytes derived from two or more different types of viruses. In certain embodiments, a sensor cell can include one or more receptors, e.g., two or more, that bind to one or more analytes derived from two or more variants or species of the same type of virus.

In certain embodiments, the heterologous receptor binds to an analyte derived from a respiratory virus, a hemorrhagic virus, a gastrointestinal virus, an exanthematous virus, a hepatitis virus, a sexually transmitted virus and/or a neurological virus.

In certain embodiments, the heterologous receptor, e.g., GPCR, binds to an analyte derived from a hemorrhagic virus. Non-limiting examples of hemorrhagic viruses include viruses from the viral families, arenaviridae, bunyaviridae, filoviridae and flaviviridae. In certain embodiments, viruses within the viral family arenaviridae include, but are not limited to, the Lassa virus, the Junin virus, the Machupo virus, the Guanarito virus, the Sabia virus, the Chapare virus and the Lujo virus. In certain embodiments, viruses within the viral family bunyaviridae include, but are not limited to, viruses within the genera Orthobunyavirus, Phlebovirus, Nairovirus, Hantavirus and Tospovirus. In certain embodiments, viruses within the viral family bunyviridae include the Rift Valley fever virus and the Crimean-Congo hemorrhagic fever virus. In certain embodiments, viruses within the viral family flaviviridae include, but are not limited to, viruses with the genus Flavivirus, e.g., the Yellow fever virus, the Dengue fever virus, the West Nile viruses and the Zika virus. In certain embodiments, viruses within the viral family filoviridae include, but are not limited to, viruses with the genera Cuevavirus, Marburgvirus and Ebolavirus. Non-limiting examples of ebolaviruses include the Ebolavirus (species Zaire ebolavirus), Sudan virus (species Sudan ebolavirus), Tal Forest virus (species Taï Forest ebolavirus, formerly Côte d'Ivoire ebolavirus), Bundibugyo virus (species Bundibugyo ebolavirus), Reston virus (species Reston ebolavirus) and Bombali virus (species Bombali ebolavirus). In certain embodiments, the hemorrhagic virus is an ebolavirus species.

In certain embodiments, the heterologous receptor, e.g., GPCR, binds to an analyte derived from a gastrointestinal virus. Non-limiting examples of gastrointestinal viruses include noroviruses, rotaviruses and astroviruses.

In certain embodiments, the heterologous receptor, e.g., GPCR, binds to an analyte derived from an exanthematous virus. Non-limiting examples of exanthematous viruses include the rubeola (measles), rubivirus (rubella), herpes (roseola), variola (smallpox), fifth disease and chikungunya viruses.

In certain embodiments, the heterologous receptor, e.g., GPCR, binds to an analyte derived from a hepatitis virus. Non-limiting examples of hepatitis viruses include Hepatitis A virus, Hepatitis B virus, Hepatitis C virus, Hepatitis D virus and Hepatitis E virus.

In certain embodiments, the heterologous receptor, e.g., GPCR, binds to an analyte derived from a neurological virus. Non-limiting examples of neurological viruses include Polio, Meningitis, encephalitis and rabies.

In certain embodiments, the heterologous receptor, e.g., GPCR, binds to an analyte derived from a sexually transmitted virus. Non-limiting examples of sexually transmitted viruses include herpes simplex viruses (HSV), papillomaviruses (HPV), human immunodeficiency virus (HIV), hepatitis B virus and cytomegalovirus.

In certain embodiments, the heterologous receptor, e.g., GPCR, binds to an analyte derived from a respiratory virus. Non-limiting examples of respiratory viruses include influenza viruses, respiratory syncytial viruses, parainfluenza viruses, metapneunovirus, rhinoviruses, coronaviruses, adenoviruses, enteroviruses and bocaviruses. Additional non-limiting examples of respiratory viruses are disclosed in Boncristiani, Encyclopedia of Microbiology. 500-518 (2009), e.g., Table 1 of Boncristiani. In certain embodiments, the influenza virus can be the Influenza A, Influenza B, Influenza C or Influenza D viruses. Additional examples of influenza viruses are disclosed in Blut et al., Transfus. Med. Hemother. 36(1):32-39 (2009); and Su et al., Virulence 8(8):1580-1591 (2017), the contents of which are hereby incorporated by reference herein in their entireties. In certain embodiments, the influenza virus can be the Parainfluenza 1, Parainfluenza 2, Parainfluenza 3 or Parainfluenza 4 viruses.

In certain embodiments, the virus is not an ebolavirus, HPV, HIV, an influenza virus, Hepatitis C virus, Hepatitis B virus, cytomegalovirus (CMV), Epstein-Barr virus (EBV), respiratory syncytial virus, norovirus, sapovirus, rubeola virus (measles), variola virus (smallpox) and viral encephalitis.

In certain embodiments, the heterologous receptor, e.g., GPCR, binds to an analyte derived from a coronavirus. In certain embodiments, the coronavirus is an alpha-coronavirus, a beta-coronavirus, a gamma-coronavirus or a delta-coronavirus. In certain embodiments, the coronavirus is an alpha-coronavirus. In certain embodiments, the coronavirus is a beta-coronavirus. In certain embodiments, the coronavirus is a gamma-coronavirus. In certain embodiments, the coronavirus is a delta-coronavirus. In certain embodiments, the coronavirus is an alpha-coronavirus such as, but not limited to, the coronavirus strains HCoV-229E and HCoV-NL63. In certain embodiments, the coronavirus is a beta-coronavirus such as, but not limited to, the coronavirus strains HCoV—OC43, HCoV—HKU1, MERS-CoV (which causes Middle East Respiratory Syndrome or MERS) and SARS-CoV (which causes severe acute respiratory syndrome or SARS). In certain embodiments, the coronavirus is MERS-CoV. In certain embodiments, the coronavirus is SARS-CoV.

In certain embodiments, the heterologous receptor, e.g., GPCR, binds to an analyte derived from SARS-CoV-2 (which causes coronavirus disease 2019 or COVID-19). In certain embodiments, the heterologous receptor binds to an analyte derived from a variant of SARS-CoV-2. In certain embodiments, the SARS-CoV-2 variant can be a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, a SARS-CoV-2 omicron variant or a SARS-CoV-2 zeta variant. Non-limiting examples of SARS-CoV-2 variants include the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants. In certain embodiments, the coronavirus is the SARS-CoV-2 B.1.1.7 variant. In certain embodiments, the coronavirus is the SARS-CoV-2 B.1.351 variant. In certain embodiments, the coronavirus is the SARS-CoV-2 P.1 variant. In certain embodiments, the coronavirus is the SARS-CoV-2 B.1.427 variant. In certain embodiments, the coronavirus is the SARS-CoV-2 B.1.429 variant. In certain embodiments, the coronavirus is the SARS-CoV-2 B.1.617.2 variant.

In certain embodiments, the heterologous receptor binds to an analyte derived from an ebolavirus. In certain embodiments, the heterologous receptor binds to an analyte derived from an ebolavirus species. In certain embodiments, the ebolavirus species is the Zaire ebolavirus, Bundibugyo ebolavirus, Sudan ebolavirus, Bombali ebolavirus, Reston ebolavirus and/or Tai Forest ebolavirus. In certain embodiments, the ebolavirus species is the Zaire ebolavirus.

In certain embodiments, the heterologous receptor, e.g., for use in detecting a viral analyte, is a fungal GPCR receptor, e.g., a fungal Ste2-type or Ste3-type GPCR from Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for use in the present disclosure comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR receptor of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for use in the present disclosure comprises a GPCR receptor of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate that is engineered by directed evolution to bind the analyte.

In certain embodiments, a GPCR for use detecting a viral analyte comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in Table 6 or Table 8. In certain embodiments, a GPCR for use in the present disclosure comprises an amino acid sequence of a GPCR disclosed herein, e.g., an amino acid sequence set forth in Table 6 or Table 8.

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in Table 6 or Table 8. In certain embodiments, a GPCR for use in the present disclosure comprises an amino acid sequence of a GPCR disclosed herein, e.g., an amino acid sequence set forth in Table 6 or Table 8.

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to comprises an amino acid sequence of a GPCR disclosed herein, e.g., an amino acid sequence set forth in Table 6 or Table 8, that has undergone directed evolution to bind a SARS-CoV-2 analyte, e.g., an epitope disclosed in Table 3. In certain embodiments, a GPCR for use in the present disclosure comprises an amino acid sequence of a GPCR disclosed herein, e.g., an amino acid sequence set forth in Table 6 or Table 8, that has undergone directed evolution to bind a SARS-CoV-2 analyte, e.g., an epitope disclosed in Table 3.

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Zygosaccharomyces rouxii. In certain embodiments, a GPCR for use in detecting SARS-CoV-2 is the GPCR of Zygosaccharomyces rouxii that has undergone directed evolution to bind the SARS-CoV-2 analyte, e.g., an epitope of the nucleocapsid protein (e.g., amino acid residues 299-310 of the nucleocapsid protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Scheffersomyces stipites. In certain embodiments, a GPCR for use in detecting SARS-CoV-2 is the GPCR of Scheffersomyces stipites that has undergone directed evolution to bind the SARS-CoV-2 analyte, e.g., an epitope of the nucleocapsid protein (e.g., amino acid residues 299-310 of the nucleocapsid protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Lodderomyces Elongisporus. In certain embodiments, a GPCR for use in detecting SARS-CoV-2 is the GPCR of Lodderomyces Elongisporus that has undergone directed evolution to bind the SARS-CoV-2 analyte, e.g., an epitope of the nucleocapsid protein (e.g., amino acid residues 299-310 of the nucleocapsid protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Candida albicans. In certain embodiments, a GPCR for use in detecting SARS-CoV-2 is the GPCR of Candida albicans that has undergone directed evolution to bind the SARS-CoV-2 analyte, e.g., an epitope of the S protein (e.g., amino acid residues GFQPTNGVGYQPYR of the S protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Baudoinia compniacensis. In certain embodiments, a GPCR for use in detecting SARS-CoV-2 is the GPCR of Baudoinia compniacensis that has undergone directed evolution to bind the SARS-CoV-2 analyte, e.g., an epitope of the S protein (e.g., amino acid residues 476-488 of the S protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of S. cerevisiae. In certain embodiments, a GPCR for use in detecting SARS-CoV-2 is the GPCR of S. cerevisiae that has undergone directed evolution to bind the SARS-CoV-2 analyte, e.g., an epitope of the S protein (e.g., amino acid residues QIAPGQTGK of the S protein).

In certain embodiments, a GPCR for use in detecting SARS-CoV-2 comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of C. lusitaniae. In certain embodiments, a GPCR for use in detecting SARS-CoV-2 is the GPCR of C. lusitaniae that has undergone directed evolution to bind the SARS-CoV-2 analyte, e.g., an epitope of the S protein (e.g., amino acid residues GWIFGTTLDSK of the S protein).

In certain embodiments, a GPCR for use in detecting an ebolavirus, e.g., the Zaire ebolavirus, comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of a GPCR disclosed herein, e.g., an amino acid sequence set forth in Table 6 or Table 8. In certain embodiments, a GPCR for use in the present disclosure comprises an amino acid sequence of a GPCR disclosed herein, e.g., an amino acid sequence set forth in Table 6 or Table 8.

In certain embodiments, a GPCR for use in detecting an ebolavirus, e.g., the Zaire ebolavirus, comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in Table 6 or Table 8 that has undergone directed evolution to bind an ebolavirus analyte, e.g., an epitope disclosed in Table 3. In certain embodiments, a GPCR for use in the present disclosure comprises an amino acid sequence of a GPCR disclosed herein, e.g., an amino acid sequence set forth in Table 6 or Table 8, that has undergone directed evolution to bind an ebolavirus analyte, e.g., an epitope disclosed in Table 3.

In certain embodiments, a GPCR for use in detecting an ebolavirus, e.g., the Zaire ebolavirus, comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of the GPCR of Candida parapsilosis. In certain embodiments, a GPCR for use in detecting SARS-CoV-2 is the GPCR of Candida parapsilosis that has undergone directed evolution to bind the ebolavirus analyte, e.g., an epitope having amino acid residues VNATEDPSSGYY.

In certain embodiments, a GPCR for use in the present disclosure comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence of a GPCR disclosed herein, e.g., a nucleotide sequence set forth in Table 7. In certain embodiments, a GPCR for use in the present disclosure comprises an amino acid sequence that is encoded by a nucleotide sequence of a GPCR disclosed herein, e.g., a nucleotide sequence set forth in Table 7.

In certain embodiments, a GPCR for use in detecting SARS-CoV-2 comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence of a GPCR disclosed herein, e.g., a nucleotide sequence set forth in Table 7. In certain embodiments, a GPCR for use in the present disclosure comprises an amino acid sequence that is encoded by a nucleotide sequence of a GPCR disclosed herein, e.g., a nucleotide sequence set forth in Table 7.

In certain embodiments, a GPCR for use in detecting an ebolavirus, e.g., the Zaire ebolavirus, comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence of a GPCR disclosed herein, e.g., a nucleotide sequence set forth in Table 7. In certain embodiments, a GPCR for use in the present disclosure comprises an amino acid sequence that is encoded by a nucleotide sequence of a GPCR disclosed herein, e.g., a nucleotide sequence set forth in Table 7.

TABLE 6

| Species | GPCR Amino Acid Sequence |
|---|---|
| Candida | MNINSTFIPDKPGDIIISYSIPGLDQPIQIPFHSLDSFQTDQAKIALVMGI |
| Albicans | TIGSCSMTLIFLISIMYKTNKLTNLKLKLKLKYILQWINQKIFTKKRNDNK |
| | QQQQQQQQQIESSSYNNTTTTTSGSYKLFLFYLNSLILLIGIIRSGCYLNY |
| | NLGPLNSLSFVFTGWYDGSSFISSDVINGFKCILYALVEISLGFQVYVMFK |
| | TSNLKIWGIMASLLSIGLGLIVVAFQINLTILSHIRFSRAISTNRSEEESS |
| | SSLSSDSVGYVINSIWMDLPTILFSISINIMTILLIGKLIIAIRTRRYLGL |
| | KQFDSFHILLIGFSQTLIIPSIILVVHYFYLSQNKDSLLQQISLLLIILML |
| | PLSSLWAQTANNTHNINSSPSLSFISRHHSSDSSRSGGSNTIVSNGGSNGG |
| | GGGGGNFPVSGIDAQLPPDIEKILHEDNNYKLLNSNNESVNDGDIIINDEG |
| | MITKQITIKRV |
| Candida | MNKIVSKLSSSDVIVTVTIPNEEDGTYEVPFYAIDNYHYSRMENAVVLGAT |
| Parapsilosis | IGACSMLLIMLIGILFKNFQRLRKSLLFNINFAILLMLILRSACYINYLMN |
| | NLSSISFFFTGIFDDESFMSSDAANAFKVILVALIEVSLTYQIYVMFKTPM |
| | LKSWGIFASVLAGVLGLATLATQIYTTVMSHVNFVNGTTGSPSQVTSAWMD |
| | MPTILFSVSINVLSMFLVCKLGLAIRTRRYLGLKQFDAFHILFIMSTQTMI |
| | IPSIILFVHYFDQNDSQTTLVNISLLLVVISLPLSSLWAQTANNVRRIDTS |
| | PSMSFISREASNRSGNETLHSGATISKYNTSNTVNTTPGTSKDDSLFILDR |
| | SIPEQRIVDTGLPKDLEKFINNDFYEDDGGMIAREVTMLKTAHNNQ |
| Baudoinia | MASNGWQNNATFDPYAQTFVLLQPDGLTPFPALLGDVLALNTVSVTQGIIY |
| Compniacensis | GTQVGISGLLLLILLIMTKPDKRRSLVFILNSLSLLLIFARNVLSCVQLTT |
| | IFYNFYNWELHWYPESPALSRAMDLSAATEVLNIPIDVAIFSSLVVQVHIV |
| | CCTIHTLVRTSALLSSAAVGLAAVAVRFALAVVNIKYSIFGINTLTEPQFN |
| | LIVHLKRVSDILTVVAIAFFSSIFVAKLGVAIHTRRTLNLKNFGAIQIIFI |
| | MGCQTMLIPLIFVIVSFYASRGSQIGSMVPTVVATFLPLSGMWASAQTNNE |
| | KMGRADQRFHRAVPVGATDESVTKARSAKASDTLDTLIGDD |
| Scheffersomyces | MDTSINTLNPANIIVNYTLPNDPRVISVPFGAFDEYVNQSMQKAIIHGVSI |
| Stipites | GSCTIMLLIILIFNVKRKKSPAFYLNSVTLTAMIIRSALNLAYLLGPLAGL |
| | SFTFSGLVTPETNFSVSEATNAFQVIVVALIEASMTFQVFVVFQSPEVKKL |
| | GIALTSISAFTGAAAVGFTINSTIQQSRIYHSVVNGTPTPTVATWSWVRDV |
| | PTILFSTSVNIMSFILILKLGFAIKTRRYLGLRQFGSLHILLMMATQTLLA |
| | PSILILVHYGYGTSSNSQLILISYLLVVLSLPVSSIWAATANNSPQLPSSA |
| | TLSFMNKTTSHESES |
| Lodderomyces | MDEAINANLVSGDIIVSFNIPGLPEPVQVPFSEFDSFHKDQLIGVIILGVT |
| Elongisporus | IGACSLLLILLLGMLYKSREKYWKSLLFMLNVCILAATILRSGCFLDYYLS |
| | DLASISYTFTGVYNGTSFASSDAANVEKTIMFALIETSLTFQVYVMFQGTT |
| | WKNWGHAVTALSGLLSVASVAFQIYTTILSHNNFNATISGTGTLTSGVWMD |
| | LPTLLFAASINFMTILLLFKLGMAIRQRRYLGLKQFDGFHILFIMFTQTLF |
| | IPSILLVIHYFYQAMSGPFIINMALFLVVAFLPLSSLWAQTANTTKKIESS |
| | PSMSFITRRKSEDESPLAANDEDRLRKFTTTLDLSGNKNNTTNNNNNSNNI |
| | NNNMSNINYPSTGLGEDDKSFIFEMEPSRERAAIEEIDLGARIDTGLPRDL |
| | EKFLVDGFDDSDDGEGMIAREVTMLKK |

TABLE 7

| Species | GPCR Nucleotide Sequence |
|---|---|
| Candida | ATGAACCCAGCTGACATCAACATCGAATACACCTTGGGTGATACTGCTTTC |
| (Clavispora) | TCTTCCACTTTCGCTGATTTCGAAGCTTGGAAAACTAGAAACACTCAATTC |
| lusitaniae | GCTATTGTCAACGGTGTCGCTTTGGCTTGTGGTATTATCTTGATGGTCGTT |
| (C1) | TCTTGGATTATTATTGTTAACAAGAGAGCTCCAATCTTCGCTATGAACCAA |
| | ACTATGTTGGTTATCATGGTTATTAAGTCCGCTATGTACTTGAAGCATATC |
| | ATGGGTCCATTGAACTCCTTGACCTTCCGTTTCACCGGTTTAATGGAAGAA |
| | TCCTGGGCTCCATACAACGTTTACGTCACTATTAACGTCTTGCATGTTTTG |
| | TTGGTCGCTGCTGTCGAATCCTCTTTGGTCTTCCAAATCCATGTTGTTTTC |
| | AAGTCTTCTAGAGCCAGAGTTGCTGGTAGAGCCATTGTTTCTGCTATGTCC |
| | ACTTTGGCCTTGTTGATCGTTTCTTTGTACTTGTACTCTACTGTTAGACAT |
| | GCTCAAACTTTGCGTGCTGAATTATCTCATGGTGACACTACCACTGTTGAA |
| | CCATGGGTCGATAACGTTCCATTGATTTTGTTTTCCGCTTCTTTGAACGTT |
| | TTGTGTTTGTTGTTGGCCTTGAAATTGGTTTTCGCTGTCAGAACCAGAAGA |
| | CATTTAGGTTTAAGACAATTCGACTCTTTCCACATCTTGATTATTATGGCC |
| | ACTCAAACTTTCGTTATCCCATCCTCTTTGGTCATCGCTAACTACAGATAC |
| | GCTTCTTCCCCATTGTTGTCTTCCATTTCCATCATCGTCGCCGTCTGTAAC |
| | TTGCCATTGTGTTCCTTGTGGGCTTGTTCTAACAACAACTCTTCCTACCCA |
| | ACTTCTTCTCAAAACACTATTTTGTCCAGATACGAAACTGAAACCTCTCAA |
| | GCTACTGACGCTTCCTCTACCACCTGTGCCGGTATTGCTGAAAAGGGTTTC |
| | GACAAGTCTCCAGACTCTCCAACTTTCGGTGACCAAGACTCCGTCTCTATC |
| | TCCCATATCTTGGACTCTTTGGAAAAGGATGTTGAAGGTGTCACCACCCAT |
| | AGATTGACTTAG |
| Komagataella | ATGGAAGAATACTCCGACTCCTTCGACCCATCCCAACAATTGTTGAACTTC |
| (Pichia) pastoris | ACTTCCTTATACGGTGAAACCGATGCTACTTTCGCTGAATTGGACGACTAC |
| (Kp) | CACTTCTACGTCGTTAAGTACGCCATCGTTTACGGTGCCAGAATTGGTGTC |
| | GGTATGTTTTGTACTTTGATGTTGTTCGTTGTTTCCAAGTCTTGGAAGACT |
| | CCAATCTTCGTCTTGAACCAATCTTCTTTGATTTTGTTGATTATTCACTCC |
| | GGTTTCTACATCCACTACTTGACCAACCAATTCTCTTCCTTGACCTACATG |
| | TTCACTAGAATCCCAAACGAAACCCATGCTGGTGTCGATTTGCGTATTAAC |
| | GTCGTTACCAACACCTTGTACGCTTTGTTGATCTTATCTATTGAAATTTCC |
| | TTAATTTACCAAGTCTTCGTTATCTTCAAAGGTGTCTACGAAAACTCTTTA |
| | AGATGGATTGTTACTATTTTCACCGCTTTATTCGCCGCCGCCGTCGTTGCT |
| | ATTAACTTCTACGTCACTACTTTGCAATCTGTCTCTATGTACAACTCTAAC |
| | GTTGACTTTCCAAGATGGGCTTCTAACGTCCCATTGATCTTGTTCGCTTCT |
| | TCTGTCAACTGGGCTTGTTTGTTGTTGTCCTTGAAGTTGTTCTTCGCTATC |
| | AAGGTTAGAAGATCTTTGGGTTTGAGACAATTCGACACTTTTCACATCTTG |
| | GCCATCATGTTCTCTCAAACTTTGATTATCCCATCCATTTTGATTGTCTTG |
| | GGTTACACTGGTACCAGAGACAGAGACTCCTTGGCTTCTTTGGGTTTCTTG |
| | TTGATCGTTGTTTCTTTGCCATTTTCCTCTATGTGGGCTGCCACTGCTAAC |
| | AACTCCAACATCCCAACCTCTACCGGTTCTTTCGCCTGGAAGAACAGATAC |
| | TCCCCATCTACTTACTCCGACGATACCACTGCTGTTTCCAAGTCCTTCACT |
| | ATTATGACCGCTAAGGATGAATGTTTCACCACTGATACCGAAGGTTCTCCA |
| | AGATTCATCAAGGGTGACAGAACCTCCGAAGATTTGCACTTCTAG |
| Candida tenuis | ATGGACTCCTACTTGTTGAACCATCCAGGTGACATCTCTTTGAACTTCGCC |
| (Cn) | TTGCCATTGTCCGATGAAGTCTACACTATTACCTTCAACGACTTAGACTCT |
| | CAATCTTCTTTTTCCATTCAATACTTGGTCATCCACTCTTGTGCCATTACC |
| | GTCTGTTTGACCTTGTTGGTTTTGTTGAACTTGTTCATCAGAAACAAGAAG |
| | ACTCCAGTCTTCGTTTTGAACCAAGTCATCTTGTTCTTCGCTATCGTCAGA |
| | TCTTCTTTGTTCATCGGTTTTATGAAGTCTCCATTGTCCACCATCACCGCC |
| | TCTTTCACCGGTATCATTTCTGATGACCAAAAACACTTCTACAAGGTCTCC |
| | GTCGCTGCTAACGCCGCTTTGATCATTTTGGTCATGTTGATTCAAGTTTCT |
| | TTCACTTACCAAATCTACATTATTTTCAGATCCCCAGAAGTTAGAAAGTTC |
| | GGTGTCTTCATGACCTCCGCCTTGGGTGTCTTGATGGCTGTTACCTTCGGT |
| | TTTTACGTTAACTCCGCTGTCGCTTCTACCAAGCAATACCAACACATCTTC |
| | TACTCTACCGACCCATACATCATGGACTCTTGGGTCACTGGTTTGCCACCA |
| | ATCTTGTACTCTGCTTCCGTCATCGCTATGTCTTTGGTCTTGGTTTTGAAG |
| | TTGGTCGCTGCTGTCAGAACCAGAAGATACTTGGGTTTGAAGCAATTCTCC |
| | TCCTACCACATCTTGTTGATTATGTTCACCCAAACCTTGTTCGTTCCAACC |
| | ATCTTGACCATCTTAGCTTACGCTTTCTACGGTTACAACGATATCTTGATC |
| | CATATTTCTACCACCATCACCGTTGTCTTGTTGCCATTCACCTCCATTTGG |
| | GCTTCTATCGCCAACAACTCTAGATCCTTGATGTCTGCCGCTTCCTTGTAC |
| | TTCTCCGGTTCCAACTCCTCTTTGTCTGAATTGTCTTCTCCATCTCCATCT |
| | GATAACGACACTTTGAACGAAAACGTCTTCGCCTTTTTTCCAGACAAGTTG |
| | CAAAAGATGAACTCTTCTGAAGCCGTTTCTGCTGTCGACAAGGTCGTTGTT |
| | CACGACCACTTTGATACCATCTCCCAAAAGTCTATCCCACACGACATCTTG |
| | GAAATTTTGCAAGGTAACGAAGGTGGTCAAATGAAGGAACACATCTCTGTC |
| | TACTCTGATGACTCTTTCTCCAAGACTACTCCACCAATTGTCGGTGGTAAC |
| | TTGTTGATCACCAACACCGACATCGGTATGAAGTAG |
| Schizosaccharomyces | ATGTACTCCTGGGACGAATTCAGATCCCCAAAGCAAGCTGAAGTTTTGAAC |
| japonicus | CAAACCGTTACCTTGGAAACTATTGTTTCCACCATTCAATTGCCAATCTCT |
| (Sj) | GAAATTGACTCCATGGAAAGAAACAGATTGTTGACCGGTATGACTGTCGCT |
| | GTTCAAGTTGGTTTAGGTTCCTTCATTTTAGTTTTGATGTGTATTTTCTCT |
| | TCCTCTGAAAAGAGAAAGAAGCCAGTCTTCATCTTCAACTTCGCTGGTAAC |
| | TTGGTTATGACTTTGAGAGCTATTTTCGAAGTTATCGTTTTGGCTTCTAAC |
| | AACTACTCTATCGCTGTTCAATACGGTTTCGCTTTTGCTGCCGTCAGACAA |
| | TACGTTCACGCCTTCAACATTATCATCTTGTTGTTGGGTCCATTCATCTTG |
| | TTCATCGCTGAAATGTCTTTGATGTTGCAAGTTAGAATCATTTGTTCCCAA |
| | CACAGACCAACTATGATTACCACCACTGTTATCTCTTGTATTTTCACTGTT |
| | GTTACCTTGGCCTTCTGGATCACCGACATGTCTCAAGAAATTGCTTACCAA |
| | TTGTTCTTGAAAAACTACAACATGAAGCAAATTGTTGGTTACTCCTGGTTG |
| | TACTTTATCGCTAAGATCACCTTCGCTGCTTCCATTATCTTCCATTCCTCC |
| | GTCTTCTCCTTCAAATTGATGCGTGCTATTTACATTCGTAGAAAGATCGGT |
| | CAATTCCCATTCGGTCCAATGCAATGTATCTTCATTGTTTCCTGTCAATGT |
| | TTGATCGTTCCAGCTATTTTCACTTTGATCGATTCTTTCACCCACACTTAC |
| | GATGGTTTCTCCTCCATGACTCAATGTTTGTTGATCATCTCCTTACCATTG |
| | TCTTCCTTGTGGGCCACCCACACCGCTCAAAAGTTGCAAACCATGAAGGAT |
| | AACACTAACCCACCATCTGGTACCCAATTAACCATCAGAGTTGATCGTACT |
| | TTCGACATGAAGTTCGTTTCCGACTCCTCTGACGGTTCTTTCACTGAAAAG |
| | ACCGAAGAAACTTTGCCA |
| Baudoinia | ATGGCTTCTAACTCTTCTAACTTCGACCCATTGACTCAATCTATCACTATC |
| compniacensis | TTGATGGCTGACGGTATCACTACTGTTTCTTTCACTCCATTGGACATCGAC |
| (Bc) | TTCTTCTACTACTACAACGTTGCTTGTTGTATCAACTACGGTGCTCAAGCT |
| | GGTGCTTGTTTGTTGATGTTCTTCGTTGTTGTTGTTTTGACTAAGGCTGTT |
| | AAGAGAAAGACTTTGTTGTTCGTTTTGAACGTTTTGTCTTTGATCTTCGGT |
| | TTCTTGAGAGCTATGTTGTACGCTATCTACTTCTTGCAAGGTTTCAACGAC |
| | TTCTACGCTGCTTTCACTTTCGACTTCTCTAGAGTTCCAAGATCTTCTTAC |
| | GCTTCTTCTGTTGCTGGTTCTGTTATCCCATTGTGTATGACTATCACTGTT |
| | AACATGTCTTTGTACTTGCAAGCTTACACTGTTTGTAAGAACTTGGACGAC |
| | ATCAAGAGAATCATCTTGACTACTTTGTCTGCTATCGTTGCTTTGTTGGCT |
| | ATCGGTTTCAGATTCGCTGCTACTGTTGTTAACTCTGTTGCTATCTTGGCT |
| | ACTTCTGCTTCTTCTGTTCCAATGCAATGGTTGGTTAAGGGTACTTTGGTT |
| | ACTGAAACTATCTCTATCTGGTTCTTCTCTTTGATCTTCACTGGTAAGTTG |
| | GTTTGGACTTTGTACAACAGAAGAAGAAACGGTTGGAGACAATGGTCTGCT |
| | GTTAGAATCTTGGCTGCTATGGGTGGTTGTACTATGGTTATCCCATCTATC |
| | TTCGCTATCTTGGAATACGTTACTCCAGTTTCTTTCCCAGAAGCTGGTTCT |
| | ATCGCTTTGACTTCTGTTGCTTTGTTGTTGCCAATCTCTTCTTTGTGGGCT |
| | GGTATGGTTACTGACGAAGAAACTTCTGCTATCGACGTTTCTAACTTGACT |
| | GGTTCTAGAACTATGTTGGGTTCTCAATCTGGTAACTTCTCTAGAAAGACT |
| | CACGCTTCTGACATCACTGCTCAATCTTCTCACTTGGACTTCTCTTCTAGA |
| | AAGGGTTCTAACGCTACTATGATGAGAAAGGGTTCTAACGCTATGGACCAA |
| | GTTACTACTATCGACTGTGTTGTTGAAGACAACCAAGCTAACAGAGGTTTG |
| | AGAGACTCTACTGAAATGGACTTGGAAGCTATGGGTGTTAGAGTTAACAAG |
| | TCTTACGGTGTTCAAAAGGCTTAG |
| Beauvaria | ATGGATGGTTCTTCTGCTCCATCTTCTCCAACTCCAGATCCAACCTTCGAC |
| bassiana | AGATTCGCCGGTAACGTCACTTTCTTCTTGGCTGACCACATCACCACTACC |
| (Bb) | TCCGTTCCAATGCCAGTCTTGAACGCCTACTACGACGAATCCTTGTGTACT |
| | ACCATGAACTACGGTGCTCAATTAGGTGCTTGTTTAGTTATGTTGGTTGTC |
| | GTTGTTGCTTTGACCCCAGCTGCTAAGTTGGCTAGAAGACCAGCTTCTGCT |
| | TTGCATTTGGTTGGTTTGTTGTTGTGTGCTGTTAGATCCGGTTTGTTGTTT |
| | GCTTACTTCGTCTCCCCAATCTCTCACTTTTACCAAGTTTGGGCTGGTGAC |
| | TTCTCTGCCGTTTCCAGAAGATACTGGGACGCTTCTTTGGCTGCCAACACT |
| | TTAGCTTTCCCATTGGTTGTCGTCGTTGAAGCTGCTTTGATCAACCAAGCT |
| | TGGACCATGGTTGCTTTCTGGCCAAGAGCCGCTAAGGCCGCTGCCTGTGCT |
| | TGTTCTGCTGTCATTGTCTTGTTGACTATTGGTACTAGATTGGCCTACACT |
| | ATCGTCCAAAACCACGCTATTGTTACTGCCGTCCCACCAGAACACTTCTTG |
| | TGGGCTATTCAATGGTCCGCTGTTATGGGTGCTGTTTCCATCTTCTGGTTT |
| | TGTGCCGTTTTCAACGTCAAGTTGGTCTGTCACTTAGTCGCTAACAGAGGT |
| | ATCTTGCCATCTATCTCTGTTGTTAACCCAATGGAAGTCTTGGTTATGACT |
| | AACGGTACCTTGATGATTATCCCATCTATCTTCGCTGGTTTGGAATGGGCT |
| | AAGTTCACCAACTTCGAATCCGGTTCTTTGACTTTGACTTCCGTTATTATT |
| | ATCTTGCCATTGGGTACTTTGGCTGCCCAACGTATTTCTGGTCAAGGTTCC |
| | CAAGGTTACCAAGCTGGTCACTTATTCCACGAACAACAACAACAACAAGCT |
| | CGTACCCGTTCCGGTGCCTTCGGTTCCGCTTCTCAACAATCCCATCCAACT |
| | AACAAGGTTCCATCCTCTATTACCTTGTCTACCTCTGGTACTCCAATTACT |
| | CCACAAATCTCTGCCGGTTCCCGTCCAGAATTACCATTGGTTGATAGATCC |
| | GAACGTTTGGACCCAATTGACTTGGAATTGGGTAGAATCGATGCTTTCAGA |
| | GGTTCTTCCGACTTCTCTCCATCCACCGCTAGACCAAAGCGTATGCAACGT |
| | GATAACTTCGCCTAG |
| Hypocrea jecorina | ATGTCTTCCTTCGACCCATACACTCAAAACATTACTATTTTGGTTTCTCCA |
| (Hj) | TCCTCTCCACCAATTTCCATTCCAATCCCAGTTATCGACGCTTTCAACGAC |
| | GAAACCGCTTCTATCATTACTAACTACGCCGCTCAATTAGGTGCTGCTTTG |
| | GCCATGTTATTAGTTTTGTTGGCCGCTACTCCAACCGCTAGATTGTTAAGA |
| | GCTGATGGTCCATCCTTGTTGCACGCTTTGGCCTTGTTAGTCTGTGTCGTC |
| | AGAACTGTCTTATTGATCTACTTCTTCTTGACCCCATTCTCTCACTTCTAC |
| | CAAGTCTGGACCGGTGACTTCTCTCAAGTTCCAGCTTGGAACTACAGAGCT |
| | TCTATTGCTGGTACCGTTTTGTCTACTTTGTTGACCGTTGTTACCGACGCT |
| | GCTTTGGTTAACCAAGCTTGGACTATGGTTTCTTTATTCGCTCCAAGAACT |
| | AAGAGAGCCGTTTGTGTTTTGTCCTTGTTAATCACCTTGTTGGCCATTTCT |
| | TTCAGAGTCGCTTACACCGTCATTCAATGTGAAGGTATCGCTGAATTGGCT |
| | GCTCCAAGACAATACGCTTGGTTGATCAGAGCCACTTTGATCTTTAACATC |
| | TGTTCCATTGCCTGGTTCTGTGCTTTGTTCAACTCTAAGTTGGTTGCTCAC |
| | TTGGTTACCAACAGAGGTGTCTTGCCATCCCGTAGAGCCATGTCCCCAATG |
| | GAAGTTTTGATTATGGCCAACGGTATCTTGATGATTGTTCCAGTTGTTTTC |
| | GCTATCTTGGAATGGCACCACTTCATTAACTTCGAAGCTGGTTCTTTAACC |
| | CCAACCTCCATCGCCATTATCTTGCCATTGTCCTCTTTGGCCGCCCAAAGA |
| | ATCGCCAACACTTCTTCCTCTTAG |
| Sporothrix | ATGAAACCCGCCGCTGGACCTGCATCTAGTCCATTCGACCCATTTAACCAA |
| scheckii | ACGTTTTACCTGACCGGTCCAGATAATACCACTGTACCAGTCTCAGTCCCA |
| (She) | CAAGTTGACTATATCTGGCATTATATTATTGGAACATCCATCAACTATGGT |
| | TCTCAGATCGGAGCCTGTTTACTTATGCTTCTTGTGATGTTGACATTGACT |
| | TCAAAGTCAAGATTTTCTCGTGCGGCCACTCTGATTAACGTAGCAAGCTTA |
| | TTGATTGGAGTAATTCGTTGTGTTCTTTTAGCTGTCTACTTTACTTCTTCT |
| | CTAACTGAATTGTATGCTCTGTTCGTTGGCGATTACAGCCAGGTCCGTAGG |
| | TCTGATCTTTGTGTCTCTGCTGTGGCAACCTTCTTTAGTCTACCACAATTA |
| | GTTCTAATAGAAGCTGCTTTGTTTCTACAGGCTTATAGTATGATCAAAATG |
| | TGGCCATCCCTGTGGAGAGCAGTGGTTTTAGCTATGTCAGTGGTGGTGGCT |
| | GTGTGTGCAATCGGTTTTAAGTTCGCGTCCGTTGTTATGCGTATGAGGTCA |
| | ACATTAACATTGGACGATTCTTTGGATTTCTGGCTAGTGGAAGTCGATCTG |
| | GCTTTTACAGCAACTACTATTTTTTGGTTTTGTTTCATCTACATTATAAGG |
| | TTGGTTATTCATATGTGGGAATATAGAAGCATTTTACCACCAATGGGGTCT |
| | GTTTCTGCTATGGAGGTTCTTGTTATGACCAATGGAGCGTTGATGTTAGTT |
| | CCAGTGATTTTCGCCGCAATAGAAATCAATGGTTTATCAAGCTTTGAATCA |
| | GGGTCACTGGTTCATACATCAGTGATTGTATTATTACCTTTAGGTAGCTTG |
| | ATAGCGCAAGCAATGACACGTCCAGATGGGTATGTCCAAAGAACGAATACA |
| | TCTGGAGCATCAGGCGCAAGTGGTGCACATCCTGGTAGAAATGGATCCGGA |
| | CACGGTGGTCATGGTGGTGCGTACTCAAGAGCCATGACTAATACCCTAAAT |
| | ACATTGGATACATTGGATACCGTAGACAGTAAGACATCCATAATGCATCAT |
| | CATCATCACCATCATAGAAACCACTCAAATGGCATGAGTAAGACGAAGGCA |
| | AATAGTGGAACATGGAGCCATGCGTCAGATGCTAACTCCACCAATGCTATG |
| | ATCAGCGGTGGTATCGCAACTCAAGTTAGGATTCAAGCTAATCAGTCAACC |
| | TTAGGAAATACGGGGATGTCCGGGGGCTCTGGAGCCCCTAATTCTCATACT |
| | CGTAATAACTCATTGGCTGCTATGGAACCAGTGGAGAAGCAACTGCATGAT |
| | ATCGATGCCACACCTTTAAGCGCATCTGATTGCAGGGTCTGGGTTGATCGT |
| | GAGGTCGAGGTCAGAAGGGACATGGTCTAG |
| Pseudogymnoascus | ATGTCCACTGCCAACGTTCATTTACCAGCTGATTTCGATCCAACTAGACAA |
| destructans | AACATCACTATCTATACCCCAGACGGTACCCCAGTTGTTGCTACCTTGCCA |
| (Pd) | ATGATCAATTTGTTTAACAGACAAAACAACGAAATCTGTGTTGTTTACGGT |
| | TGTCAATTGGGTGCCTCTTTAATTATGTTCTTGGTTGTTTTGTTGACCACC |
| | AGAGTTTCCAAGAGAAAATCTCCAATCTTCGTCTTGAACGTTTTGTCTTTG |
| | ATTATTTCTTGTTTAAGATCCTTGTTGCAAATTTTATACTATATTGGTCCA |
| | TGGACCGAGATCTACAGATACTTGTCTTTCGATTACTCTACTGTCCCAGCT |
| | TCCGCTTACGCTAATTCTGTTGCTGCCACTTTATTAACCTTATTCTTATTG |
| | ATTACCATTGAAGCTTCTTTAGTTTTACAAACTAACGTTGTCTGCAAGTCT |
| | ATGTCTTCTCACATTCGTTGGCCAGTTACTGCTTTGTCCATGGTTGTCTCT |
| | TTATTGGCTATTTCTTTTAGATTCGGTTTGACCATCCGTAACATCGAAGGT |
| | ATCTTAGGTGCTACTGTCAAATCCGACTCCTTAATGTTCTCTGGTGCCTCT |
| | TTGATCTCTGAAACTGCTTCTATCTGGTTCTTCTGCACTATTTTCGTTATT |
| | AAATTGGGTTGGACCTTGTACCAAAGAAAGAAGATGGGTTTGAAGCAATGG |
| | GGTCCAATGCAAATTATCACTATCATGGCTGGTTGCACCATGTTGATCCCA |
| | TCCTTGTTCACTGTTTTGGAATTCTTCCCTGAAGAAACTTTCTACGAGGCC |
| | GGTACTTTGGCTATCTGTTTGGTTGCTATTTTGTTGCCATTATCTTCCGTC |
| | TGGGCTGCCGCTGCTATTGATGGTGATGAACCAGTCCGTCCACATGGTTCT |
| | ACCCCAAAATTCGCTTCTTTCAACATGGGTTCCGACTACAAATCTTCTTCT |
| | GCTCACTTGCCAAGATCTATTAGAAAGGCCTCCGTCCCAGCTGAACATTTA |
| | TCTAGAACTTCTGAAGAAGAGTTAGGTGACGACGGTACTTTGAACAGAGGT |
| | GGTGCCTACGGTATGGACAGAATGTCCGGTTCTATCTCCCCTAGAGGTGTC |
| | AGAATTGAAAGAACTTACGAAGTTCATACCGCTGGTAGAGGTGGTTCTATC |
| | GAGAGAGAGGACATCTTCTAG |
| Lodderomyces | ATGGACGAAGCAATCAATGCAAACCTTGTTTCTGGAGATATTATAGTCTCT |
| elongisporous | TTTAACATTCCTGGTTTGCCAGAACCGGTACAAGTGCCATTCAGCGAATTT |
| (Le) | GATTCGTTTCATAAAGACCAGCTCATTGGAGTCATCATTCTTGGAGTCACT |
| | ATTGGAGCATGCTCGCTTTTGTTGATATTGCTACTTGGAATGTTATACAAG |
| | AGCCGTGAAAAGTATTGGAAATCACTATTATTTATGCTCAATGTATGCATC |
| | TTGGCTGCCACAATCTTAAGGAGCGGTTGCTTCTTAGACTATTATCTAAGT |
| | GATTTGGCCAGTATCAGTTATACATTTACTGGAGTATACAATGGTACCAGC |
| | TTTGCTAGCTCTGACGCGGCAAATGTGTTCAAGACTATTATGTTTGCCTTG |
| | ATTGAAACTTCGTTAACCTTTCAAGTGTATGTCATGTTTCAAGGGACCACT |
| | TGGAAAAATTGGGGCCATGCTGTCACTGCATTATCGGGTCTCTTGTCTGTT |
| | GCCTCAGTGGCGTTCCAGATCTACACCACGATTTTATCCCACAATAATTTC |
| | AATGCTACAATCTCGGGAACCGGTACATTAACTTCAGGTGTTTGGATGGAC |
| | TTACCAACACTCTTGTTTGCCGCAAGTATCAATTTTATGACCATTTTGTTG |
| | TTATTTAAGTTGGGAATGGCCATTAGACAAAGAAGGTATTTAGGTTTAAAA |
| | CAGTTTGATGGGTTCCATATCTTATTCATCATGTTTACCCAAACATTGTTC |
| | ATACCCTCGATTTTGCTTGTGATCCACTACTTTTACCAGGCAATGTCTGGA |
| | CCATTCATCATCAACATGGCGTTGTTCTTGGTGGTGGCATTCTTGCCATTG |
| | AGTTCATTATGGGCACAAACTGCAAACACTACTAAAAAGATTGAATCTTCG |
| | CCAAGTATGAGCTTTATTACTAGACGAAAATCAGAGGATGAGTCACCACTG |
| | GCTGCTAACGACGAGGATAGGTTACGAAAATTCACCACAACTTTGGATTTG |
| | TCGGGCAACAAGAACAATACAACAAACAATAATAACAATAGCAACAACATT |
| | AACAACAATATGAGCAACATCAACTACCCTTCTACAGGACTGGGAGAAGAC |
| | GATAAATCCTTTATATTTGAGATGGAACCCAGTCGGGAAAGAGCTGCAATA |
| | GAAGAGATTGATCTTGGAGCAAGGATCGATACCGGTTTGCCCAGAGATTTA |
| | GAGAAATTTCTAGTTGATGGGTTTGACGATAGTGATGACGGAGAAGGAATG |
| | ATAGCCAGAGAAGTGACTATGTTGAAAAAATAG |
| Penicillium | ATGGCTACCTCTTCCCCAATCCAACCATTTGACCCATTCACCCAAAACGTT |
| chrysogenum | ACCTTCCGTTTGCAAGACGGTACCGAATTCCCAGTTTCTGTCAAGGCTTTG |
| (Pr) | GACGTCTTCGTCATGTACAACGTTAGAGTCTGTATTAACTACGGTTGTCAA |
| | TTCGGTGCCTCCTTCGTCTTGTTAGTCATTTTAGTCTTGTTAACTCAATCC |
| | GACAAGAGAAGATCTGCTGTCTTCATTTTGAACGGTTTGGCTTTGTTCTTG |
| | AACTCTTCTAGATTGTTGTTTCAAGTTATTCACTTCTCCACTGCCTTCGAA |
| | CAAGTCTACCCATACGTCTCTGGTGACTACTCCTCTGTCCCATGGTCCGCT |
| | TACGCTATCTCCATTGTCGCTGTTGTTTTGACTACCTTGGTCGTTGTTTGT |
| | ATCGAAGCTTCTTTGGTTATTCAAGTTCACGTTGTCTGTTCCACCTTGAGA |
| | CGTAGATACAGACACCCATTATTAGCTATTTCTATTTTGGTCGCTTTGGTT |
| | CCAATCGGTTTCAGATGTGCTTGGATGGTCGCTAACTGTAAGGCTATTATT |
| | AAATTGACCTACACCAACGACGTTTGGTGGATCGAATCTGCTACTAACATC |
| | TGTGTCACTATCTCCATCTGTTTCTTCTGTGTTATCTTCGTTACCAAGTTG |
| | GGTTTCGCCATCAAGCAAAGAAGAAGATTGGGTGTTAGAGAATTCGGTCCA |
| | ATGAAGGTTATTTTCGTCATGGGTTGTCAAACTATGGTTGTTCCAGCTATT |
| | TTCTCCATCACCCAATACTACGTCGTCGTCCCAGAATTCTCCTCTAACGTC |
| | GTTACTTTGGTTGTCATTTCTTTACCATTATCTTCCATTTGGGCCGGTGCT |
| | GTCTTGGAAAACGCTAGAAGAACCGGTTCCCAAGATAGACAAAGAAGACGT |
| | AACTTGTGGAGAGCTTTGGTTGGTGGTGCTGAATCCTTGTTATCCCCAACT |
| | AAGGACTCTCCAACCTCTTTGTCTGCTATGACTGCTGCTCAAACCTTATGT |
| | TACTCTGATCACACCATGTCCAAGGGTTCTCCAACTTCCAGAGACACCGAT |
| | GCTTTCTACGGTATCTCCGTTGAACACGACATCTCCATTAACAGAGTTCAA |
| | CGTAACAACTCCATCGTCTAG |
| Aspergillus oryzae | ATGGACTCTAAGTTCGACCCATACTCTCAAAACTTGACTTTCCACGCTGCT |
| (Ao) | GACGGTACCCCATTTCAAGTTCCAGTCATGACCTTGAACGACTTTTACCAA |
| | TACTGTATTCAAATTTGTATCAACTACGGTGCTCAATTCGGTGCTTCCGTC |
| | ATCATTTTCATTATCTTGTTGTTATTGACTAGACCAGACAAAAGAGCTTCT |
| | TCTGTTTTCTTCTTAAACGGTGGTGCCTTGTTGTTGAACATGGGTAGATTG |
| | TTGTGTCACATGATTTACTTCACTACTGACTTCGTCAAGGCTTACCAATAC |
| | TTCTCTTCTGATTACTCTAGAGCCCCAACCTCTGCCTACGCTAACTCCATT |
| | TTGGGTGTCGTCTTGACCACCTTGTTGTTGGTTTGTATCGAAACCTCCTTG |
| | GTTTTACAAGTCCAAGTCGTCTGTGCTAACTTGAGACGTAGATACAGAACC |
| | GTCTTATTGTGTGTTTCTATCTTGGTCGCCTTGATCCCAGTCGGTTTGAGA |
| | TTGGGTTACATGGTTGAAAACTGTAAGACTATTGTTCAAACTGATACCCCA |
| | TTGTCTTTGGTTTGGTTGGAATCTGCTACTAACATCGTCATTACCATCTCC |
| | ATCTGTTTCTTCTGTTCTATCTTCATCATCAAGTTGGGTTTCGCCATTCAC |
| | CAAAGAAGAAGATTGGGTGTCAGAGATTTCGGTCCAATGAAGGTCATTTTC |
| | GTCATGGGTTGTCAAACTTTGACTGTTCCAGCTTTGTTGTCTATTTTGCAA |
| | TACGCTGTCTCTGTCCCAGAATTGAACTCTAACATTATGACTTTGGTTACT |
| | ATCTCTTTGCCATTGTCCTCCATTTGGGCTGGTGTTTCTTTGACCCGTTCT |
| | TCCTCCACCGAAAACTCTCCATCCAGAGGTGCTTTGTGGAACCGTTTGACC |
| | GACTCTACCGGTACCAGATCTAACCAAACCTCTTCCACCGACACCGCCGTC |
| | GCTATGACCTACCCATCTAACAAGTCTTCTACTGTCTGTTACGCCGATCAA |
| | TCTTCTGTCAAGAGACAATACGATCCAGAACAAGGTCACGGTATCTCTGTT |
| | GAACACGATGTTTCTGTCCACTCCTGTCAAAGATTGTAG |
| Paracoccidioides | ATGGCACCCTCATTCGACCCCTTCAACCAAAACGTGGTCTTCCACAAGGCC |
| brasiliensis | GACGGAACCCCATTCAACGTCTCAATCCATGAACTAGACGATTTCGTGCAG |
| (Pb) | TACAATACCAGAGTCTGCATCAACTACTCTTCCCAGCTCGGAGCATCTGTC |
| | ATTGCGGGACTCATGCTTGCCATGCTGACACACTCAGAAAAGCGTCGTCTG |
| | CCGGTTTTCTTCCTAAACACATTCGCACTGGCCATGAACTTTGCCCGCCTG |
| | CTCTGCATGACCATCTACTTCACCACGGGCTTCAACAAGTCCTATGCCTAC |
| | TTTGGTCAGGATTACTCCCAGGTGCCTGGGAGCGCCTACGCAGCCTCTGTC |
| | TTGGGCGTTGTCTTCACCACTCTGCTGGTAATCAGCATGGAAATGTCCCTC |
| | CTGATCCAAACGAGGGTTGTCTGCACGACCCTTCCGGATATCCAACGTTAT |
| | CTACTCATGGCAGTTTCCTCCGCGATTTCCCTGATGGCCATCGGGTTCCGC |
| | CTTGGCTTAATGGTTGAGAACTGCATTGCTATTGTGCAGGCGTCGAATTTC |
| | GCGCCTTTTATCTGGCTTCAAAGCGCCTCGAACATCACCATTACGATCAGC |
| | ACATGTTTCTTCAGTGCCGTCTTTGTTACGAAATTGGCATATGCACTCGTC |
| | ACTCGTATACGACTAGGCTTGACCAGGTTTGGTGCTATGCAGGTTATGTTT |
| | ATCATGTCCTGCCAGACTATGGTGATCCCAGCCATCTTCTCAATTCTCCAA |
| | TACCCACTCCCCAAGTACGAAATGAACTCCAACCTCTTTACGCTGGTGGCC |
| | ATTTTCCTTCCTCTTTCCTCGCTATGGGCTTCAGTGGCTACGAAATCCAGT |
| | TTCGAGACGTCTTCTTCCGGCCGCCATCAGTATCTTTGGCCAAGCGAACAG |
| | AGCAATAACGTCACCAATTCGGAAATTAAGTATCAGGTCAGCTTCTCTCAG |
| | AACCACACTACGCTGCGGTCTGGAGGGTCTGTGGCCACGACACTCTCTCCG |
| | GACCGGCTCGACCCGGTTTATTCTGAAGTTGAAGCTGGCACAAAGGCCTAG |
| Aspergillus | ATGAACTCCACCTTCGACCCATGGACCCAAAACATTACTTTGACTCAATCC |
| fischeri | GACGGTACCACTGTCATCTCCTCTTTGGCTTTGGCCGATGACTACTTGCAC |
| (Af) | TACATGATTAGATTGGGTATCAACTACGGTGCCCAATTGGGTGCTTGTGCT |
| | GTTTTGTTGTTGGTTTTGTTATTGTTGACTAGACCAGAAAAGAGAGTTTCT |
| | TCTGTCTTCGTTTTGAACGTCGCTGCTTTGTTGGCTAACATCATCAGATTG |
| | GGTTGTCAATTGTCCTACTTCTCTACCGGTTTCGCTAGAATGTACGCCTTG |
| | TTGGCCGGTGACTTCTCCAGAGTCTCTCGTGGTGCTTACGCCGGTCAAGTT |
| | ATGGCCTCCGTCTTCTTCACCATTGTCTTCATTTGTGTTGAAGCTTCTTTG |
| | GTTTTGCAAGTTCAAGTCGTCTGTTCTAACTTGAGAAGACAATACAGAATC |
| | TTGTTATTGGGTGCTTCCACTTTGGCTGCCTTGGTTCCAATTGGTGTTCGT |
| | TTGACTTACTCCGTTTTAAACTGTATGGTTATTATGCACGCTGGTACTATG |
| | GACCACTTGGATTGGTTGGAATCTGCTACCAACATCGTTACTACCGTTTCT |
| | ATTTGTTTCTTCTGTGCTGTTTTCGTTGTCAAATTAGGTTTGGCTATCAAG |
| | ATGAGAAAGCGTTTGGGTGTCAAACAATTCGGTCCAATGAGAGTTATCTTC |
| | ATCATGGGTTGTCAAACCATGACCATCCCAGCTATTTTCGCTATTTGTCAA |
| | TACTTCTCTAGAATTCCAGAATTTTCTCATAACGTTTTGACTTTGGTTATC |
| | ATCTCTTTGCCATTGTCTTCTATCTGGGCCGGTTTTGCTTTGGTCCAAGCC |
| | AACTCTACCGCCAGATCTACCGAATCTAGACATCATTTGTGGAACATTTTG |
| | TCTTCCGATGGTGCTACCAGAGACAAGCCATCCCAATGTGTTTCTTCTCCA |
| | ATGACCTCTCCAACCACTACCTGTTACTCCGAACAATCCACCTCTAAGCCA |
| | CAACAAGACCCAGAAAACGGTTTTGGTATTTCTGTTGCCCACGATATTTCC |
| | ATCCACTCTTTCAGAAAGGACGCCCACGGTGATATTTAG |
| Yarrowia | ATGCAATTGCCACCACGTCCAGACTTCGACATTGCCACTTTGGTTGCCTCT |
| lipolytica | ATCACTGTTCCAGAAACTGAATTGGTCTTGGGTCAAATGCCATTGGGTGCT |
| (Y1) | TTAGAACAATTGTACCAAAACAGATTGCGTTTGGCTATTTTGTTCGGTGTC |
| | AGAGTCGGTGCTGCTGTTTTGACCTTGATTGCTATGCACTTAATCTCCAAG |
| | AAGAACAGAACCAAGATCTTGTTCTTGGCTAACCAAATGTCTTTGATCATG |
| | TTGATCATCCATGCTGCTTTGTACTTCAGATTCTTGTTGGGTCCATTCGCC |
| | TCCATGTTGATGATGGTTGCTTACATCGTTGATCCAAGATCTAACGTCTCT |
| | AACGATATCTCTGTTTCTGTTGCCACCAACGTTTTCATGATGTTGATGATT |
| | ATGTCCGTCCAATTGTCTTTGGCTGTTCAAACCCGTTCTGTTTTCCACGCT |
| | TGGTTGAAGTCTCGTATTTACGTTACCGTTGGTTTAATCTTGTTGTCCTTG |
| | GTCGTCTTCGTCTTCTGGACCACCCACACTATCGTTTCTTGTATCGTTTTA |
| | ACCCATCCAACTAGAGACTTGCCATCTATGGGTTGGACTAGATTAGCTTCT |
| | GACGTTTCCTTCGCTTGTTCTATCTCTTTCGCTTCTTTGGTCTTGTTGGCT |
| | AAGTTGGTCACCGCCATCAGAGTTAGAAAGACCTTGGGTAAGAAGCCATTG |
| | GGTTACACCAAGGTTTTGGTCATCATGTCCACTCAATCTTTAGTCGTTCCA |
| | TCTATCTTGATTATCGTTAACTACGCTTTGCCAGAAAAAAACTCTTGGATC |
| | TTGTCTGGTGTCGCTTACTTGATGGTTGTTTTGTCCTTACCATTGTCCTCC |
| | ATTTGGGCTACCGCCGTCCATGACGACGAAATGCAATCCAACTACTTGTTG |
| | TCTGCCTTGAAAGATGGTCACGTTCAACCATCCGAATCTAAGTTGAAGACT |
| | GTTTTCTTGAACAGATTGAGACCATTCTCTACTACCACTAACAGAGACGAT |
| | GAATCCTCTGTTGATTCCCCAGCCATGCCATCTCCAGAATCTGATGTTACC |
| | TTCTTGAACACTGGTTTCGAATGTGACGAAAAGATGTAG |
| Tuber | ATGGAGCAAATCCCAGTCTACGAGCGTCCAGGTTTCAACCCACACAAGCAA |
| melanosporum | AACATTACCTTGTTCAAGCATGATGGTTCTACTGTTACTGTCGGTTTGCAT |
| (Tm) | GAGTTGGACGCCATGTTCACTCATTCCATCAGAGTTGCTGTCGTCTTCGCC |
| | TCTCAAATTGGTGCTTGTGCTTTGTTGTCTGTTATCGTTGCTATGGTCACC |
| | AAGAGAGAAAAGAGACGTGCTTTGTTCTTCTTGCACATTATTTCCTTGTTG |
| | TTGGTCGTTGTTCGTTCCGTCTTGCAAATCTTGTACTTCGTCGGTCCATGG |
| | GCTGAAACTTATAATTACGTCGCCTACTACTATGAAGACATTCCTTTGTCT |
| | GACAAATTGATTTCCATTTGGGCTGGTATTATCCAATTGATTTTGAATATC |
| | TGTATTTTGTTATCTTTGATCTTGCAAGTTCGTGTCGTTTACGCCACCTCT |
| | CCAAAATTGAACACTATTATGACTTTAGTCTCTTGTGTTATCGCTTCTATT |
| | TCTGTCGGTTTCTTCTTTACTGTCATCGTTCAAATTTCTGAGGCTATTTTA |
| | AACGGTGTTGGTTACGACGGTTGGGTTTACAAAGTCCATAGAGGTGTCTTC |
| | GCTGGTGCTATCGCCTTCTTCTCTTTCATCTTCATCTTTAAGTTGGCCTTC |
| | GCTATCAGAAGAAGAAAGGCTTTGGGTTTGCAAAGATTCGGTCCATTGCAA |
| | GTTATCTTCATCATGGGTTGTCAAACTATGATTGTTCCAGCTATCTTTGCT |
| | ACTTTGGAAAACGGTGTTGGTTTCGAAGGTATGTCCTCTTTGACTGCTACC |
| | TTGGCTGTCATTTCCTTACCATTGTCTTCTATGTGGGCCGCCGCTCAAACC |
| | GACGGTCCATCTCCACAATCCACTCCAAGAGACGGTTATAGAAGATTCTCT |
| | ACTCGTAGATCTGCCTTGAACAGATCTGACCCATCTGGTGGTAGATCTGTT |
| | GACATGAACACCTTGGACTCTACCGGTAACGATTCCTTAGCTTTGCACGTT |
| | GATAAGACTTTTACTGTTGAATCTTCCCCATCCTCCCAATCTCAAGCTGGT |
| | CCACACAAGGAAAGAGGTTTCGAATTCGCCTAG |
| Dactylellina | ATGGACCACAACACCCAACACTTCAACAGACCTGAATACATTGAAATCCCA |
| haptotyla | GTTCCACCATCTAAGGGTTTCAACCCACACACCAACCCTGCTTTCTTCATC |
| (Dh) | TACCCAGACGGTTCTAATATGACCTTTTGGTTCGGTCAAATCGACGATTTC |
| | AGACGTGACCAATTATTCACTAACACCATCTTTTCCATTCAAATTGGTGCC |
| | GCTTTGGTCATCTTATGTGTCATGTTTTGTGTTACCCACGCTGATAAGCGT |
| | AAAACCATTGTCTACTTGTTAAACGTTTCCAACTTGTTCGTTGTTATCATT |
| | AGAGGTGTTTTCTTTGTTCATTACTTCATGGGTGGTTTGGCCAGAACCTAT |
| | ACCACTTTCACCTGGGATACTTCTGATGTTCAACAATCTGAGAAGGCTACT |
| | TCCATTGTCTCCTCTATTTGTTCTTTGATTTTGATGATCGGTACTCAAATC |
| | TCCTTATTGTTGCAAGTCAGAATCTGTTACGCTTTGAACCCAAGATCCAAG |
| | ACCGCTATCTTGGTTACTTGTGGTTCTATTTCCGGTATTGCTACCACTGCT |
| | TATTTATTGTTGGGTGCTTACACTATTCAATTGAGAGAAAAGCCACCAGAC |
| | ATGAAGTTCATGAAGTGGGCTAAGCCAGTTGTTAACGCTTTGGTTGCCTTG |
| | TCCATTGTCTCCTTTTCTGGTATTTTCTCTTGGAGAATGTTCCAATCTGTC |
| | AGAAACAGAAGAAGAATGGGTTTCACTGGTATCGGTTCCTTGGAATCTTTG |
| | TTGGCTTCTGGTTTCCAATGTTTAGTCTTCCCTGGTTTGGTTACTACCGCT |
| | TTGACCGTCGCCGGTTCCACTTGGTATATCGCTGTTAACTTAACTACTCCA |
| | TCTGACTTGACCGCTATTTACAACTGTTCCGCTTTTTTCGCTTATGCTTTC |
| | TCCATTCCATTGTTAAAGGAAAGAGCTCAAGTTGAAAAGACCATTTCTGTT |
| | GTCATTGCTATCGCTGGTGTCTTAGTCGTTGCTTACGGTGACGGTGCTGAC |
| | GACGGTTCCACCTCTAACGGTGAAAAGGCTAGATTGGGTGGTAACGTCTTG |
| | ATCGGTATCGGTTCTGTCTTGTATGGTTTATACGAAGTCTTGTATAAGAAG |
| | TTATTATGTCCACCATCTGGTGCTTCCCCAGGTAGATCTGTTGTTTTCTCT |
| | AATACCGTTTGTGCTTGCATCGGTGCTTTCACTTTGTTATTCTTGTGGATC |
| | CCATTGCCATTGTTGCACTGGTCCGGTTGGGAAATTTTTGAATTGCCAACC |
| | GGTAAGACTGCTAAGTTATTGGGTATTTCCATTGCCGCTAACGCCACCTTC |
| | TCTGGTTCTTTCTTGATCTTAATTTCTTTGACTGGTCCAGTTTTGTCCTCT |
| | GTTGCCGCCTTGTTGACCATTTTCTTGGTTGCTATTACTGACAGAATTTTA |
| | TTCGGTAGAGAATTGACTTCTGCTGCCATTTTGGGTGGTTTGTTGATCATC |
| | GCTGCCTTCGCTTTGTTATCTTGGGCTACTTGGAAGGAAATGATTGAAGAG |
| | AACGAGAAGGATACTATCGATTCCATCTCTGACGTTGGTGACCACGATGAC |
| | TAG |
| Candida (Pichia) | ATGAAGTCCTGCTCCATCGGTTTCGGTATCCCATTCATTAATGAACCAAAC |
| guilliermondii | TTCGAAACTGTTTCTATTTTGACCATGGACGTTTCTTTCATTGACGCTGAC |
| (Cgu) | GTCAATCCTGACAATATCTTGTTGAACTTCACCATTCCTGGTTACCAAAAC |
| | GGTTTCTCTGTTCCAATGGTTGTTATTAACGAATTGCAAAAGTCTCAAATG |
| | AAATACGCTATTGTTTACGGTTGTGGTGTCGGTGCCTCCTTGATTTTGTTG |
| | TTTGTCGTCTGGATTTTGTGTTCTAGAAAGACTCCATTGTTTATCATGAAC |
| | AACATTCCATTAGTTTTGTACGTCATCTCCTCTTCTTTGAACTTGGCTTAC |
| | ATTACCGGTCCATTGTCTTCTGTTTCCGTCTTCTTGACCGGTATCTTGACT |
| | TCTCACGATGCCATTAACGTCGTTTACGCTTCCAACGCTTTGCAAATGTTG |
| | TTGATCTTTTCTATCCAATCTACCATGGCCTACCACGTTTACGTTATGTTC |
| | AAATCTCCACAAATTAAATACTTGAGATACATGTTAGTCGGTTTCTTGGGT |
| | TGTTTACAAATTGTCACCACCTGTTTATACATCAACTACAATGTTTTGTAC |
| | TCTCGTAGAATGCACAAATTGTACGAAACTGGTCAAACCTACCAAGATGGT |
| | ACCGTTATGACTTTCGTTCCATTCATCTTGTTCCAATGTTCTGTCAACTTC |
| | TCTTCTATTTTCTTGGTTTTGAAGTTGATTATGGCCATTAGAACCAGACGT |
| | TACTTGGGTTTGCGTCAATTCGGTGGTTTTCATATTTTGATGATCGTTTCT |
| | TTACAAACTATGTTGGTCCCATCTATTTTGGTTTTGGTTAACTACGCCGCT |
| | CATAAGGCTGTTCCTTCCAACTTGTTATCTTCCGTTTCTATGATGATCATT |
| | GTTTTGTCTTTACCAGCTTCTTCTATGTGGGCCGCTGCTGCTAACGCCTCT |
| | TCTGCCCCTTCCTCCGCTGCTTCCTCCTTGTTCAGATACACCACTTCTGAT |
| | TCCGATAGAACTTTGGAAACTAAATCTGACCACTTCATCATGAAGCATGAG |
| | TCCCACAACTCTTCTCCAAATTCCTCCCCATTGACTTTGGTTCAAAAGAGA |
| | ATTTCTGATGCCACCTTAGAATTACCAAAAGAGTTAGAAGACTTGATCGAC |
| | TCCACCTCCATC |
| Aspergillus | ATGGCTACCCACAACCAAATCTCTGATCAATGTCAATGGTCTTACCCAGAA |
| nidulans | GTCTTCACCACTCAAGCTGTCGAAGAACCAACCGCCGAACCAGCTTCTTAC |
| (An) | CACTTGCACTCTACCTTGACTATTATGGCTTCTAACTTCGACCCATGGAAC |
| | CAAACCATTACCTTCAGATTGGAAGACGGTACTCCATTCGACATTTCTGTC |
| | GACTACTTGGACGGTATCTTGCAATACTCTATCAGAGCTTGTGTCAACTAC |
| | GCTGCTCAATTGGGTGCTTCTGTCATTTTGTTTGTTATCTTGGTCTTGTTG |
| | ACTAGAGCCGAAAAAAGAGCTTCTTGTTTGTTCTGGTTAAACTCCTTAGCT |
| | TTGTTGTTGAACTTCGCCAGATTGTTGTGTGACGTCTTGTTCTTCACCGGT |
| | AACTTCGTCAGAATTTACACTTTGATCTCCGCTGACGAATCTAGAGTTACT |
| | GCTTCCGACTTGGCTACTTCCATCGTCGGTGCTATCATGACCGCTTTGTTG |
| | TTGACCACTATTGAAATTTCTTTGGTTTTGCAAGTCCAAGTCGTTTGTTCT |
| | AACTTGAGAAGAATCTACAGAAGAGCCTTGTTGTGTGTTTCCGCCGTCGTT |
| | GCCACTGCTACCATTGCTATTAGATACTCCTTGTTGGCTGTCAACATTAGA |
| | GCTATTTTGGAATTCTCCGACCCAACTACTTACAACTGGTTGGAATCTTTA |
| | GCTACCGTCGCCTTGACCATCTCCATCTGTTACTTCTGTGTCATCTTCGTC |
| | ACCAAGTTAGGTTTCGCTATTAGATTGAGAAGAAAGTTGGGTTTATCTGAA |
| | TTGGGTCCAATGAAGGTCGTCTTCATCATGGGTTGTCAAACCTTGGTCATC |
| | CCAGGTAAAAGAACCTTGTCTTCTTTGATTCCACCAGTCATTGTTTCTATT |
| | ACTCACTACGTCTCCGACGTCCCAGAATTGCAAACTAACGTTTTGACTATC |
| | GTCGCCTTGTCCTTGCCATTGTCCTCTATTTGGGCTGGTACCACCATTGAC |
| | AAGCCAGTCACTCACTCTAACGTTAGAAACTTGTGGCAAATCTTGTCCTTC |
| | TCTGGTTACAGACCAAAGCAATCTACCTACATTGCTACCACTACTACCGCT |
| | ACTACCAACGCTAAGCAATGTACCCACTGTTACTCTGAATCTAGATTGTTG |
| | ACTGAAAAGGAATCTGGTCGTAACAACGACACTTCTTCTAAGTCTTCCTCC |
| | CAATACGGTATCGCTGTCGAACACGATATTTCCGTTAGATCTGCTCGTCGT |
| | GAATCTTTTGACGTCTAG |
| Zygosaccharomyces | ATGAGTGAGATTAACAATTCTACCTACAATCCAATGAATGCATATGTAACG |
| rouxii | TTTACATCAATATATGGTGATGATACTATGGTACGTTTCAAAGATGTGGAA |
| (Zr) | TTGGTAGTTAACAAAAGGGTTACAGAAGCCATTATGTTCGGCGTCAAAGTT |
| | GGTGCAGCTTCGTTGACACTCATCATCATGTGGATGATCTCTAAGAAAAGA |
| | ACAACACCGATATTTATCATAAATCAGTCTTCGCTTGTATTTACCATAATA |
| | CATGCTTCGCTTTATTTTGGGTACCTTTTGTCAGGATTTGGTAGTATAGTT |
| | TACAATATGACATCGTTCCCGCAGTTAATAAGCTCCAATGACGTTCGTGTG |
| | TACGCAGCTACAAATATTTTTGAGGTCCTGTTGGTAGCATCTATCGAAATC |
| | TCTCTGGTTTTTCAGGTCAAAGTTATGTTTGCCAACAATAATGGTCGAAGA |
| | TGGACTTGGTGTTTGATGGTAGTTTCCATAGGGATGGCACTAGCTACTGTA |
| | GGACTTTATTTTGCCACTGCCGTTGAGTTGATCAGAGCTGCTTACAGCAAT |
| | GATACTGTTAGCCGCCATGTTTTTTACAATGTTTCTCTGATCTTACTAGCG |
| | TCATCTGTCAATCTAATGACACTAATGCTAGTGGTAAAATTAGTATTAGCG |
| | ATCAGATCAAGAAGATTTTTGGGGTTAAAACAGTTTGACAGTTTCCACATA |
| | TTACTTATAATGTCTTGCCAGACTCTAATAGCACCTTCCATTCTATTCATT |
| | TTGGGTTGGACCTTAGACCCTCATACTGGTAATGAGGTTTTAATTACAGTT |
| | GGTCAATTGCTAATAGTACTGTCATTACCGCTGTCATCTATGTGGGCTACA |
| | ACCGCTAACAATACCAGTTCATCTAGTAGTTCGGTGTCCTGTAATGACAGC |
| | TCTTTTGGTAATGACAATCTCTGTTCCAAGAGTTCGCAATTTAGAAGAACT |
| | TTTATGAATAGATTCCGTCCCAAGTCGGTTAATGGTGACGGTAATTCTGAA |
| | AATACCTTTGTTACAATTGATGATTTGGAAAAAAGCGTTTTTCAAGAATTA |
| | TCAACACCTGTTAGCGGAGAATCAAAGATAGATCATGATCATGCAAGTAGT |
| | ATTTCATGTCAAAAGACATGTAATCATGTTCATGCTTCGACAGTGAATTCA |
| | GATAAGGGATCTTGGTCCTCTGATGGTAGTTGTGGCAGTTCTCCGTTAAGA |
| | AAGACTTCCACCGTTAATTCTGAAGATTTACCTCCACATATATTGAGCGCC |
| | TACGATGACGATCGAGGTATAGTAGAAAGTAAAAAAATTATCCTAAAGAAA |
| | TTATAG |
| Geotrichum | ATGGCCGAAGACTCCATCTTCCCAAACAACTCCACCTCTCCATTGACCAAC |
| candidum | CCAATTGTTGTTGAAACCATTAAGGGTACCGCTTACATTCCATTACACTAC |
| (Gc) | TTGGATGATTTGCAATACGAAAAGATGTTGTTGGCTTCCTTGTTCTCCGTT |
| | AGAATTGCTACTTCCTTCGTTGTTATTATTTGGTACTTCGTCGCTGTCAAC |
| | AAGGCTAAGAGATCTAAGTTTTTGTACATTGTCAACCAAGTTTCTTTGTTG |
| | ATCGTTTTTATCCAATCCATTTTGTCTTTGATTTACGTCTTCTCCAACTTC |
| | TCCAAGATGTCTACCATTTTGACCGGTGATTACACCGGTATCACTAAGAGA |
| | GACATTAACGTCTCTTGTGTTGCCTCCGTTTTCCAATTCTTGTTCATCGCT |
| | TGTATCGAATTGGCTTTGTTCATCCAAGCTACTGTCGTTTTCCAAAAATCT |
| | GTTAGATGGTTGAAGTTTTCCGTTTCTTTGATCCAAGGTTCCGTCGCTTTG |
| | ACTACTACCGCCTTGTACATGGCCATTATTGTCCAATCCATCTACGCTACT |
| | TTGAACCCATACGCTGGTAACTTGATTAAAGGTCGTTTCGGTTACTTATTA |
| | GCTTCTTTGGGTAAGATTTTCTTCTCTATTTCTGTTACTTCTTGTATGTGT |
| | ATCTTCGTTGGTAAGTTGGTCTTTGCTATTCACCAAAGAAGAACTTTGGGT |
| | ATTAAGCAATTCGACGGTTTGCAAATTTTGGTCATTATGTCTACTCAATCC |
| | ATGATCATCCCAACTATTATCGTCTTGATGTCTTTTTTGAGACGTAACGCT |
| | GGTTCTGTTTACACCATGGCTACCTTGTTGGTCGCTTTGTCCTTGCCATTG |
| | TCCTCCTTGTGGGCTGAAGCCAAGACTACCAGAGACTCTGCTTCTTACACC |
| | GCTTACAGACCATCTGGTTCTCCAAACAACCGTTCTTTGTTCGCCATCTTC |
| | TCTGATAGATTGGCTTGTGGTTCTGGTAGAAACAACAGACACGATGATGAT |
| | TCTAGAGGTAACGGTTCTGTTAACGCCAGAAAGGCTGACGTCGAATCTACT |
| | ATCGAAATGTCCTCTTGTTACACTGATTCCCCAACCTACTCCAAGTTCGAA |
| | GCTGGTTTGGACGCTAGAGGTATCGTCTTCTACAACGAACACGGTTTGCCA |
| | GTTGTCTCCGGTGAAGTTGGTGGTTCTTCCTCCAACGGTACTAAGTTGGGT |
| | TCTGGTCATAAGTACGAAGTCAACACTACTGTTGTTTTGTCTGATGTTGAC |
| | TCTCCATCTCCAACCGACGTCACCCGTAAGTAG |
| Torulaspora | ATGTCTGACTCCGCCCAAAACTTGTCCGATTTGGCCTTCAACTCTTCTTAT |
| delbrueckii | AACCCATTGGACTCCTTTATTACCTTTACCTCTATCTACGGTGATAACACT |
| (Td) | GCTGTTAAGTTCTCCGTTTTACAAGACATGGTTGACGTTAATACTAATGAA |
| | GCCATCGTTTACGGTACCCGTTGTGGTGCTTCTGTCTTGACCCAAATTATC |
| | ATGTGGATGATTTCTAAAAACAGAAGAACCCCAGTCTTTATTATTAACCAA |
| | GTTTCTTTGACTTTGATTTTAATTCACTCTGCCTTGTACTTCAAGTACTTG |
| | TTGTCTGGTTTCGGTTCCGTTGTCTACGGTTTGACTGCTTTCCCACAATTG |
| | ATTAAGCCAGGTGATTTGAGAGCTTTCGCTGCTGCTAACATCGTTATGGTC |
| | TTGTTGGTCGCTTCTATTGAAGCTTCCTTAATCTTCCAAGTCAAAGTTATC |
| | TTCACCGGTGATAACATGAAGAGAGTCGGTTTAATCTTGACTATTATTTGT |
| | ACTTGTATGGGTTTAGCTACTGTTACCATGTACTTTATTACTGCCGTCAAG |
| | TCTATTGTCTCTTTGTACCGTGACATGTCTGGTTCCTCCACCGTTTTATAT |
| | AACGTTTCTTTAATTATGTTGGCTTCCTCCATCCACTTTATGGCTTTGATC |
| | TTGGTTGTCAAATTGTTCTTGGCTGTTAGATCTAGAAGATTCTTGGGTTTG |
| | AAACAATTCGATTCTTTCCACATTTTGTTGATCATCTCTTGTCAAACTTTG |
| | TTGGTTCCATCTTTATTATTCATTATTGCTTACTCTTTTCCATCTTCTAAG |
| | AACATTGAATCTTTGAAGGCTATCGCTGTTTTGACCGTCGTTTTGTCTTTG |
| | CCATTGTCTTCTATGTGGGCTACTGCTGCTAATAACTTCACTAACTCTTCC |
| | TCCTCCGGTTCCGACTCCGCTCCAACCAATGGTGGTTTCTACGGTAGAGGT |
| | TCTTCCAACTTGTATCCTGAAAAGACTGATAACAGATCCCCAAAGGGTGCC |
| | AGAAACGCTTTATACGAATTAAGATCTAAGAACAATGCTGAGGGTCAAGCT |
| | GATATTTACACCGTTACCGATATTGAAAACGATATTTTCAACGATTTGTCC |
| | AAGCCAGTTGAGCAAAACATTTTCTCTGATGTTCAAATTATTGATTCTCAT |
| | TCTTTGCATAAGGCTTGTTCTAAAGAAGACCCAGTCATGACTTTGTACACT |
| | CCAAACACTGCTATTGAAGGTGAGGAGAGAAAATTGTGGACTTCTGACTGT |
| | TCCTGTTCCACTAACGGTTCCACCCCAGTTAAGAAGAAGTCCACCGGTGAA |
| | TACGCCAATTTACCACCACACTTATTAAGATATGATGAAAACTACGATGAA |
| | GAAGCTGGTGGTAGACGTAAGGCCTCCTTGAAATGGTAG |
| Neurospora | ATGGCGTCCTCTTCCTCACCACCTGCAGACATTTTCTCAGGGATCACGCAA |
| crassa | TCACTAAATAGTACACACGCGACGCTTACACTACCGATTCCGCCAGCGGAC |
| (Nc) | AGGGATCATCTGGAAAATCAAGTATTATTTTTGTTTGACAATCACGGTCAG |
| | TTACTTAATGTAACTACAACTTACATTGACGCTTTTAACAATATGCTGGTC |
| | TCTACTACTATAAACTATGCAACGCAAATTGGAGCTACTTTTATAATGCTA |
| | GCCATTATGTTATTAATGACTCCCAGAAGGAGGTTCAAACGTTTACCAACA |
| | ATTATTAGCTTGTTAGCCTTATGTATTAATTTGATCAGGGTGGTTTTGCTG |
| | GCCCTGTTTTTTCCTTCTCACTGGACAGACTTCTACGTGTTGTATTCCGGT |
| | GACTGGCAGTTTGTACCTCCAGGGGATATGCAAATATCTGTTGCTGCTACG |
| | GTTTTGTCTATCCCAGTGACGGCATTATTATTGAGCGCATTGATGGTTCAA |
| | GCCTGGTCAATGATGCAATTATGGACACCACTGTGGAGGGCACTAGTGGTA |
| | CTAGTGTCCGGGCTATTGTCACTGGTAACTGTGGCAATGAGTTTCGCGAAT |
| | TGCATTTTCCAAGCGAAAAATATTTTGTATGCCGACCCTTTACCCTCCTAC |
| | TGGGTCAGAAAATTGTACTTAGCATTAACGACTGGGTCTATAAGTTGGTTC |
| | ACATTCCTTTTTATGATAAGATTGGTTATGCATATGTGGACAAACAGATCT |
| | ATATTACCAAGCATGAAGGGTTTGAAGGCTATGGATGTATTGATTATTACG |
| | AATTCTATATTGATGTTAATCCCAGTGTTGTTTGCAGGCTTGGAATTTCTG |
| | GATAGTGCCTCTGGATTTGAGTCCGGGTCTTTGACTCAAACCTCTGTAGTG |
| | ATTGTCCTGCCTTTGGGTACTTTAGTAGCACAAAGAATAGCTACGAGGGGT |
| | TACATGCCCGATAGTCTGGAGGCTTCTAGCGGACCAAATGGTTCATTGCCG |
| | TTATCTAATTTAAGTTTCGCTGGAGGGGGCGGTGGTGGTTCTGGGGGACAT |
| | AAAGATAAAGAAAACGGTGGCGGTATTATACCGCCTACTACGAACAATACT |
| | GCTGCTACTAATTTTTCTTCATCAATCGCGTGTTCTGGTATATCTTGTTTA |
| | CCAAAAGTCAAAAGAATGACCGCGAGTTCAGCCTCAAGTAGCCAGAGACCG |
| | TTGTTGACAATGACTAACTCAACCATAGCGAGTAATGACAGTTCAGGTTTC |
| | CCTTCTCCTGGCATACATAATACCACTACTACGACAACACAATACCAATAT |
| | TCCATGGGAATGAACATGCCGAACTTTCCTCCAGTCCCGTTCCCAGGTTAC |
| | CAGTCACGTACTACCGGTGTTACTTCCCATATTGTGTCCGACGGTAGACAT |
| | CACCAGGGTATGAACAGGCACCCATCTGTTGACCATTTTGATAGGGAACTT |
| | GCTAGGATTGATGATGAAGATGACGATGGTTACCCTTTCGCATCAAGTGAA |
| | AAGGCCGTTATGCACGGAGACGATGACGACGATGTGGAAAGGGGACGTCGT |
| | AGAGCTCTACCACCATCCTTAGGTGGAGTTAGAGTTGAAAGGACGATCGAG |
| | ACCAGGAGCGAGGAACGTATGCCATCTCCGGACCCATTGGGTGTTACGAAG |
| | CCTAGATCATTCGAGTAG |
| Saccharomyces | ATGTCTGATGCGGCTCCTTCATTGAGCAATCTATTTTATGATCCAACGTAT |
| cerevisiae | AATCCTGGTCAAAGCACCATTAACTACACTTCCATATATGGGAATGGATCT |
| (Sc) | ACCATCACTTTCGATGAGTTGCAAGGTTTAGTTAACAGTACTGTTACTCAG |
| | GCCATTATGTTTGGTGTCAGATGTGGTGCAGCTGCTTTGACTTTGATTGTC |
| | ATGTGGATGACATCGAGAAGCAGAAAAACGCCGATTTTCATTATCAACCAA |
| | GTTTCATTGTTTTTAATCATTTTGCATTCTGCACTCTATTTTAAATATTTA |
| | CTGTCTAATTACTCTTCAGTGACTTACGCTCTCACCGGATTTCCTCAGTTC |
| | ATCAGTAGAGGTGACGTTCATGTTTATGGTGCTACAAATATAATTCAAGTC |
| | CTTCTTGTGGCTTCTATTGAGACTTCACTGGTGTTTCAGATAAAAGTTATT |
| | TTCACAGGCGACAACTTCAAAAGGATAGGTTTGATGCTGACGTCGATATCT |
| | TTCACTTTAGGGATTGCTACAGTTACCATGTATTTTGTAAGCGCTGTTAAA |
| | GGTATGATTGTGACTTATAATGATGTTAGTGCCACCCAAGATAAATACTTC |
| | AATGCATCCACAATTTTACTTGCATCCTCAATAAACTTTATGTCATTTGTC |
| | CTGGTAGTTAAATTGATTTTAGCTATTAGATCAAGAAGATTCCTTGGTCTC |
| | AAGCAGTTCGATAGTTTCCATATTTTACTCATAATGTCATGTCAATCTTTG |
| | TTGGTTCCATCGATAATATTCATCCTCGCATACAGTTTGAAACCAAACCAG |
| | GGAACAGATGTCTTGACTACTGTTGCAACATTACTTGCTGTATTGTCTTTA |
| | CCATTATCATCAATGTGGGCCACGGCTGCTAATAATGCATCCAAAACAAAC |
| | ACAATTACTTCAGACTTTACAACATCCACAGATAGGTTTTATCCAGGCACG |
| | CTGTCTAGCTTTCAAACTGATAGTATCAACAACGATGCTAAAAGCAGTCTC |
| | AGAAGTAGATTATATGACCTATATCCTAGAAGGAAGGAAACAACATCGGAT |
| | AAACATTCGGAAAGAACTTTTGTTTCTGAGACTGCAGATGATATAGAGAAA |
| | AATCAGTTTTATCAGTTGCCCACACCTACGAGTTCAAAAAATACTAGGATA |
| | GGACCGTTTGCTGATGCAAGTTACAAAGAGGGAGAAGTTGAACCCGTCGAC |
| | ATGTACACTCCCGATACGGCAGCTGATGAGGAAGCCAGAAAGTTCTGGACT |
| | GAAGATAATAATAATTTATAG |
| Schizosaccharomyces | ATGCGTGAACCATGGTGGAAGAACTACTACACCATGAACGGTACCCAAGTC |
| octosporus | CAAAACCAATCCATCCCAATTTTGTCCACCCAAGGTTACATTCAAGTTCCA |
| (So) | TTGTCCACCATCGATAAGGCTGAAAGAAACAGAATTTTGACTGGTATGACC |
| | GTTTCTGCTCAATTGGCCTTGGGTGTCTTGATCATGGTCATGTCTATTTTG |
| | TTGTCCTCCCCAGAAAAGAGAAAGACCCCAGTTTTCATCGTCAACTCTGCC |
| | TCTATCATTTCCATGTGTATTAGAGCTATCTTGATGATTGTCAACTTGTGT |
| | TCTGAATCCTACTCTTTGGCTGTTATGTACGGTTTCGTCTTCGAATTGGTT |
| | GGTCAATACGTTCACGTTTTTGACATTTTGGTTATGATTATTGGTACCATC |
| | ATCATTATTACCGCTGAAGTTTCCATGTTGTTGCAAGTCAGAATTATTTGT |
| | GCTCACGACAGAAAGACTCAAAGAATTGTTACCTGTATCTCTTCTGGTTTA |
| | TCCTTGATCGTCGTTGCCTTCTGGTTCACTGATATGTGTCAAGAAATTAAG |
| | TACTTGTTGTGGTTGACCCCATACAACAACCACCAAATCTCTGGTTACTAC |
| | TGGGTTTACTTCGTCGGTAAGATCTTGTTCGCCGTTTCCATTATGTTCCAC |
| | TCTGCCGTCTTCTCCTACAAGTTGTTCCACGCTATCCAAATTAGAAAGAAG |
| | ATTGGTCAATTCCCATTCGGTCCAATGCAATGTATTTTAATTATTTCCTGT |
| | CAATGTTTGTTCGTTCCAGCTATTTTCACTATCATCGACTCTTTCATCCAC |
| | ACTTACGACGGTTTTTCCTCCATGACCCAATGTTTGTTGATCGTCTCTTTG |
| | CCATTGTCCTCCTTGTGGGCCTCTTCCACTGCTTTAAAGTTGCAATCTTTG |
| | AAGTCTACCACCTCTCCAGGTGACACTACTCAAGTTTCCATTAGAGTCGAC |
| | AGAACCTACGACATCAAGAGAATCCCAACTGAAGAATTGTCTTCTGTTGAC |
| | GAAACCGAAATCAAGAAGTGGCCATAG |
| Candida albicans | ATGAATATCAATTCAACTTTCATACCTGATAAACCAGGCGATATAATTATT |
| (Ca) | AGTTATTCAATTCCAGGATTAGATCAACCAATTCAAATTCCTTTCCATTCA |
| | TTAGATTCATTTCAAACCGATCAAGCTAAAATAGCTTTAGTCATGGGGATA |
| | ACTATTGGGAGTTGTTCAATGACATTAATTTTTTTGATTTCTATAATGTAT |
| | AAAACTAATAAATTAACAAATTTAAAATTAAAATTAAAATTAAAATATATC |
| | TTGCAATGGATAAATCAAAAAATCTTCACCAAAAAAAGGAATGACAACAAA |
| | CAACAACAACAACAACAACAACAACAAATTGAATCATCATCATATAACAAT |
| | ACTACTACTACGCTGGGGGGTTATAAATTATTTTTATTTTATCTTAATTCA |
| | TTGATTTTATTAATTGGTATTATTCGATCAGGTTGTTATTTAAATTATAAT |
| | TTAGGTCCATTAAATTCACTTAGTTTTGTATTTACTGGTTGGTATGATGGA |
| | TCATCATTTATATCATCCGATGTAACTAATGGATTTAAATGTATTTTATAT |
| | GCTTTAGTGGAAATTTCATTAGGTTTCCAAGTTTATGTGATGTTCAAAACT |
| | TCAAATTTAAAAATTTGGGGGATAATGGCATCATTATTATCAATTGGTTTA |
| | GGATTGATTGTTGTTGCCTTTCAAATCAATTTAACAATTTTATCTCATATT |
| | CGATTTTCCCGGGCTATATCAACTAACAGAAGTGAAGAAGAATCATCATCA |
| | TCATTATCATCTGATTCGGTTGGGTATGTGATTAATTCAATATGGATGGAT |
| | TTACCAACAATATTATTTTCCATTAGTATTAATATAATGACAATATTATTG |
| | ATTGGTAAACTTATAATTGCTATTAGAACAAGACGTTATTTAGGATTGAAA |
| | CAATTTGATAGTTTCCATATTTTATTAATTGGTTTCAGTCAAACATTAATT |
| | ATTCCTTCAATTATTTTGGTGGTTCATTATTTTTATTTATCACAAAATAAA |
| | GATTCTTTATTACAACAAATTAGTCTTTTATTGATTATTTTAATGTTACCA |
| | TTAAGTTCTTTATGGGCTCAAACTGCTAATAATACTCATAATATTAATTCA |
| | TCTCCAAGTTTATCATTCATATCTCGTCATCATCTGTCTGATAGTAGTCGT |
| | AGTGGTGGTTCCAATACAATTGTTAGTAATGGTGGTAGTAATGGTGGTGGT |
| | GGTGGTGGTGGGAATTTCCCTGTTTCAGGTATTGATGCACAATTACCACCT |
| | GATATTGAAAAAATCTTACATGAAGATAATAATTATAAATTACTTAATAGT |
| | AATAATGAAAGTGTAAATGATGGAGATATTATCATTAATGATGAAGGTATG |
| | ATTACTAAACAAATCACCATCAAAAGAGTGTAG |
| Schizosaccharomyces | ATGAGACAACCATGGTGGAAAGACTTTACTATTCCCGATGCATCCGCAATT |
| pombe | ATTCACCAAAATATTACCATTGTCTCTATTGTAGGAGAGATTGAAGTGCCA |
| (Sp) | GTTTCAACAATTGATGCATATGAAAGAGATAGACTTTTAACTGGAATGACT |
| | TTGTCTGCCCAACTTGCTTTAGGAGTCCTTACCATTTTGATGGTTTGTCTA |
| | TTGTCATCATCCGAAAAACGAAAACACCCAGTTTTTGTTTTTAATTCGGCA |
| | AGTATTGTTGCAATGTGTCTTCGGGCCATTTTGAATATAGTGACCATATGC |
| | AGCAATAGCTACAGTATCCTGGTTAATTACGGGTTTATCTTAAACATGGTT |
| | CATATGTATGTCCATGTGTTTAATATTTTAATTTTGTTGCTTGCACCGGTC |
| | ATCATTTTTACTGCTGAGATGAGCATGATGATTCAAGTTCGTATAATTTGT |
| | GCACATGATAGAAAGACACAAAGGATAATGACTGTTATTAGTGCCTGCTTA |
| | ACTGTTTTGGTTCTCGCATTTTGGATTACTAACATGTGTCAACAGATTCAG |
| | TATCTGTTATGGTTAACTCCACTTAGCAGCAAGACCATTGTTGGATACTCT |
| | TGGCCCTACTTTATTGCTAAAATACTTTTTGCTTTTAGCATTATTTTTCAC |
| | AGTGGTGTTTTTTCATACAAACTCTTTCGTGCCATATTAATACGGAAAAAA |
| | ATTGGGCAATTTCCATTTGGTCCGATGCAGTGTATTTTAGTTATTAGCTGC |
| | CAATGTCTTATTGTTCCAGCTACCTTTACTATAATAGATAGTTTTATCCAT |
| | ACGTATGATGGCTTTAGCTCTATGACTCAATGTCTGCTAATCATTTCTCTT |
| | CCTCTTTCGAGTTTATGGGCGTCTAGTACAGCTCTGAAATTGCAAAGCATG |
| | AAAACTTCATCTGCGCAAGGAGAAACCACCGAGGTTTCGATTAGAGTTGAT |
| | AGAACGTTTGATATCAAACATACTCCCAGTGACGATTATTCGATTTCTGAT |
| | GAATCTGAAACTAAAAAGTGGACGTAG |
| Vanderwaltozyma | ATGTCAGGAATTGATGATATGGGTGATAAACCAGATATTTTAGGTTTATTT |
| polyspora 2 | TATGATGCTAACTATGATCCAGGTCAAGGTATACTCACATTTATTTCAATG |
| (Vp2) | TACGGGAATACTACTATAACTTTTGATGAGTTACAGTTAGAGGTCAATAGT |
| | TTAATTACAAGTGGTATTATGTTCGGCGTCAGATGTGGTGCTGCTTGTTTG |
| | ACATTGTTAATAATGTGGATGATTTCTAAGAATAAGAAGACTCCAATTTTT |
| | ATTATTAATCAATGCTCGCTAATCCTTATTATTATGCATTCAGGTTTATAT |
| | TTTAAGAATATTCTATCAAATTTGAATTCTTTATCATATATCTTAACTGGG |
| | TTTACTCAAAATATCACTAAAAATAATATACATGTCTTTGGTGCCGCTAAT |
| | ATTATTCAAGTTTTATTAGTAGCAACCATTGAACTGTCGTTAGTGTTTCAA |
| | ATTCGAGTCATGTTTAAAGGTGACAGTTTTAGAAAAGCTGGTTACGGTTTG |
| | TTGTCAATTGCGTCTGGTTTGGGTATAGCTACTGTCGTCATGTATTTTTAC |
| | TCTGCCATTACAAATATGATTGCTGTTTATAATCAAACTTACAACTCCACT |
| | GCTAAATTATTTAACGTTGCAAACATTCTTCTGTCTACATCGATAAATTTT |
| | ATGACGGTAGTATTAATTGTTAAATTATTTTTGGCTGTTAGATCAAGAAGA |
| | TATTTGGGTTTAAAGCAGTTCGATAGTTTCCATATTTTATTGATTATGTCA |
| | TGTCAAACATTGATTGTACCATCAATTCTTTTTATCTTATCATACGCTTTA |
| | AGTACTAAGCTGTACACTGATCATTTAGTTGTCATTGCAACTTTATTAGTC |
| | GTTCTATCTTTACCATTATCTTCGATGTGGGCAAGCGCTGCAAATAATTCT |
| | CCTAAACCAAGCTCGTTTACAACCGATTATTCAAACAAGAATCCTAGTGAC |
| | ACACCAAGCTTCTACAGTCAAAGTATTAGTTCCTCGATGAAAAGCAAATTC |
| | CCAAGCAAATTCATACCCTTCAATTTCAAGTCTAAAGACAATTCTTCTGAC |
| | ACTAGATCAGAAAATACATATATTGGCAATTATGACATGGAAAAGAATGGA |
| | TCACCAAATCACTCTTATTCTTCCAAAGATCAAAGTGAAGTTTACACTATA |
| | GGTGTAAGCTCTATGCACACAGATATAAAGTCACAAAAGAATATCAGTGGA |
| | CAGCATTTATATACCCCAAGTACAGAGATTGATGAAGAAGCTAGAGACTTC |
| | TGGGGGGCAGAGCTGTTAATAATTCAGTTCCAAATGACTATCAACCATCT |
| | GAGTTACCAGCATCGATTCTTGAAGAATTGAATTCACTGGATGAAAATAAT |
| | GAAGGTTTCTTGGAGACAAAAAGAATAACATTTAGAAAACAATAG |
| Scheffersomyces | ATGGATACTAGTATCAATACTCTCAACCCTGCGAATATCATTGTCAACTAC |
| stipitis | ACCTTGCCAAATGATCCTAGAGTAATTAGTGTCCCATTTGGAGCTTTTGAC |
| (Ss) | GAATATGTTAACCAATCTATGCAAAAGGCCATTATCCATGGAGTTTCCATT |
| | GGTTCATGCACCATAATGCTTTTAATTATTTTGATCTTCAATGTCAAACGC |
| | AAGAAGTCGCCAGCTTTCTATCTTAATTCGGTTACGTTGACTGCAATGATT |
| | ATTCGGTCTGCTCTTAATTTGGCATATTTGCTAGGTCCTTTGGCTGGATTA |
| | AGTTTTACGTTCTCCGGCTTGGTAACTCCAGAAACCAATTTCTCTGTCTCT |
| | GAAGCCACCAATGCTTTCCAGGTTATTGTTGTTGCTCTTATCGAGGCGTCC |
| | ATGACATTTCAGGTGTTCGTCGTCTTCCAATCACCAGAAGTGAAGAAGTTG |
| | GGTATAGCTCTTACCTCCATATCTGCATTCACGGGTGCTGCTGCTGTAGGA |
| | TTTACTATCAATAGTACAATCCAACAATCGAGAATTTATCATTCAGTTGTC |
| | AATGGAACTCCTACGCCAACGGTCGCTACCTGGTCTTGGGTTAGAGATGTG |
| | CCTACGATACTTTTTTCTACTTCGGTTAACATAATGTCTTTCATCTTGATT |
| | CTCAAGTTAGGGTTTGCCATAAAGACAAGAAGATACCTTGGCCTTCGGCAA |
| | TTTGGCAGTTTGCACATCTTATTGATGATGGCTACTCAAACATTATTGGCC |
| | CCATCTATTCTCATTCTTGTACATTACGGATATGGCACATCTCTGAATAGC |
| | CAGCTCATTCTTATAAGTTACTTGCTTGTTGTTTTGTCTTTACCAGTATCC |
| | TCTATCTGGGCAGCAACAGCCAACAATTCTCCTCAACTTCCATCTTCCGCA |
| | ACTCTTTCATTCATGAACAAAACGACCTCTCACTTTTCTGAAAGCTAG |
| Candida glabrata | ATGGAGATGGGCTACGATCCAAGAATGTATAATCCAAGAAATGAATACTTG |
| (Cg) | AATTTCACGTCGGTATATGATGTAAATGACACAATCAGATTTTCGACTCTG |
| | GACGCCATTGTAAAAGGATTGCTTAGAATTGCCATTGTTCATGGAGTTAGA |
| | TTGGGAGCAATATTCATGACGTTAATAATAATGTTTATCTCATCAAATACA |
| | TGGAAAAAACCCATATTTATAATTAACATGGTGTCGTTGATGTTAGTTATG |
| | ATTCATTCCGCACTTAGCTTCCATTACCTTTTATCGAATTATTCTTCAATT |
| | TCTTATATACTGACAGGGTTTCCTCAGTTGATTACAAGCAATAATAAACGA |
| | ATTCAAGATGCAGCGAGTATAGTCCAAGTTTTATTGGTTGCTGCGATAGAA |
| | GCATCATTGGTATTTCAGATTCATGTTATGTTTACGATTGAAAACATTAAG |
| | CTTATTAGAGAAATAGTACTCTCTATATCGATAGCAATGGGATTGGCAACA |
| | GTGGCTACATATCTTGCTGCAGCAATAAAGCTGATAAGAGGACTGCATGAT |
| | GAGGTAATGCCACAAACACATCTTATTTTCAATTTATCTATAATATTGCTT |
| | GCATCCTCCATAAATTTTATGACATTTATATTGGTCATTAAACTTTTCTTC |
| | GCTATTAGATCTAGAAGATATCTCGGTCTTCGTCAATTCGATGCTTTTCAT |
| | ATTTTATTAATCATGTTCTGCCAGTCATTATTGATACCCTCAGTATTATAT |
| | ATTATAGTTTACGCGGTTGATAGCAGATCTAATCAGGATTATCTGATTCCA |
| | ATTGCCAATTTATTTGTTGTTTTATCTTTGCCATTATCCTCTATCTGGGCT |
| | AACACATCAAATAACTCATCCAGATCTCCAAAATATTGGAAAAACTCTCAA |
| | ACGAATAAGAGCAATGGGTCTTTTGTCTCTTCAATATCTGTCAATAGTGAC |
| | TCACAAAACCCTTTGTACAAAAAGATTGTACGTTTTACATCAAAAGGCGAC |
| | ACTACCCGTAGTATTGTAAGTGATTCAACATTAGCAGAGGTGGGAAAATAC |
| | TCTATGCAAGACGTTAGCAATTCAAACTTTGAATGTCGAGACCTTGATTTT |
| | GAGAAGGTAAAACATACTTGCGAAAATTTTGGCAGAATATCTGAAACATAT |
| | AGTGAGTTAAGTACTTTAGATACCACTGCCCTCAATGAGACTCGGTTGTTT |
| | TGGAAACAACAAAGTCAGTGTGACAAATAG |
| Vanderwaltozyma | ATGAGTTCCCAATCACACCCACCGCTAATCGATTTATTTTACGATTCCAGT |
| polyspora 1 | TATGACCCTGGTGAAAGTTTAATTTATTACACATCCATCTATGGTAATAAT |
| (Vp1) | ACATACATAACTTTTGATGAACTCCAGACGATAGTGAACAAGAAGGTCACA |
| | CAAGGTATCTTATTTGGTGTCAGATGTGGTGCTGCTTTCCTGATGTTGGTA |
| | GCAATGTGGTTGATTTCCAAAAATAAAAGATCTAGAATTTTCATTACCAAC |
| | CAATGTTGTCTGGTCTTCATGATAATGCATTCTGGTCTTTATTTTAGGTAC |
| | CTGCTTTCAAGGTACGGTTCAGTTACTTTCATTCTAACAGGGTTCCAACAA |
| | CTGCTTACAAGAAATGACATTCATATTTATGGAGCTACTGATTTTATCCAA |
| | GTAGCTTTGGTAGCTTGCATAGAATTATCTCTTATTTTCCAAATAAAAGTG |
| | ATATTCGCTGGTACAAACTATGGTAAGTTGGCTAATTATTTCATCACTCTA |
| | GGTTCATTATTGGGTTTAGCCACCTTTGGTATGTACATGCTTACTGCTATT |
| | AACGGTACAATAAAATTATACAATAACGAATATGACCCAAACCAAAGGAAA |
| | TACTTTAACATTTCTACAATATTGCTTGCATCATCAATTAATATGCTAACG |
| | CTGATACTTATATTGAAGCTGGTGGCAGCAATTAGAACAAGACGTTACTTA |
| | GGTTTGAAGCAATTCGATAGTTTTCACATCCTATTAATCATGTCGACTCAA |
| | ACATTAATAATTCCTTCTATCTTATTTATTCTATCATACAGTTTGAGAGAG |
| | GATATGCATACTGATCAATTAATAATCATCGGAAATCTGATCGTGGTATTG |
| | TCATTACCATTGTCCTCAATGTGGGCTTCGTCTCTAAACAATTCAAGTAAA |
| | CCTACATCTTTGAATACTGATTTCTCAGGGCCAAAATCAAGTGAAGAAGGG |
| | ACAGCAATAAGTTTGCTATCACAAAACATGGAACCATCAATAGTCACTAAA |
| | TATACAAGAAGATCACCTGGGTTATACCCAGTAAGCGTGGGTACACCAATT |
| | GAAAAAGAAGCATCATACACTCTTTTTGAAGCTACTGACATTGATTTTGAA |
| | AGCAGTAGTAACGATATCACAAGGACTTCATAG |
| Zygosaccharomyces | ATGTCTGGTTTGGCTAACAACACCTCTTACAACCCATTGGAATCTTTCATT |
| bailii | ATTTTCACTTCTGTTTACGGTGGTGATACCATGGTTAAGTTCGAAGACTTG |
| (Zb) | CAATTAGTCTTCACCAAGCGTATTACTGAAGGTATTTTGTTCGGTGTCAAG |
| | GTTGGTGCCGCTTCTTTGACTATGATTGTTATGTGGATGATTTCCAGAAGA |
| | AGAACCTCCCCAATCTTCATCATGAACCAATTGTCTTTGGTTTTCACCATC |
| | TTGCACGCTTCTTTTTACTTTAAGTACTTATTGGACGGTTTCGGTTCTATT |
| | GTCTACACTTTGACCTTGTTCCCACAATTAATTACTTCCTCTGACTTGCAC |
| | GTTTTCGCTACTGCTAACGTTGTTGAAGTCTTATTGGTTTCTTCCATCGAA |
| | GCCTCTTTGGTTTTCCAAGTCAACGTCATGTTCGCTGGTTCTAACCACAGA |
| | AAGTTCGCTTGGTTGTTGGTCGGTTTCTCTTTGGGTTTGGCTTTGGCCACT |
| | GTCGCTTTGTACTTCGTTACTGCTGTCAAGATGATCGCTTCCGCTTACGCT |
| | TCTCAACCACCAACTAACCCAATCTACTTCAACGTTTCCTTGTTCTTGTTG |
| | GCTGCCTCCGTTTTCTTGATGACTTTAATGTTGACCGTCAAGTTGATCTTG |
| | GCTATCAGATCCAGAAGATTCTTGGGTTTGAAGCAATTCGACTCTTTCCAC |
| | ATTTTGTTGATTATGTCTTGTCAAACTTTGATCGCTCCATCTGTTTTGTAC |
| | ATCTTGGGTTTTATTTTGGATCACAGAAAGGGTAACGACTACTTGATTACC |
| | GTCGCTCAATTGTTGGTCGTTTTGTCTTTGCCATTGTCCTCCATGTGGGCC |
| | ACTACTGCTAACGATGCTTCCTCCGGTACTTCTATGTCTTCCAAGGAATCC |
| | GTCTACGGTTCTGATTCCTTATACTCTAAGTCTAAGTGTTCCCAATTCACC |
| | AGAACCTTCATGAACAGATTCTCTACTAAGCCAACTAAGAACGACGAAATT |
| | TCTGATTCCGCTTTCGTCGCTGTTGATTCCTTGGAAAAGAACGCTCCACAA |
| | GGTATCTCTGAACACGTTTGTGAATTCCCACAATCTGACTTATCTGATCAA |
| | GCTACTTCCATCTCCTCCAGAAAAAAGGAAGCTGTTGTTTACGCTTCCACT |
| | GTTGATGAAGATAAGGGTTCTTTCTCCTCTGACATCAACGGTTACACTGTT |
| | ACCAACATGCCATTGGCTTCCGCTGCTTCTGCTAACTGTGAAAACTCCCCA |
| | TGTCACGTTCCAAGACCATACGAAGAAAACGAAGGTGTCGTCGAAACCAGA |
| | AAAATTATTTTGAAGAAGAACGTCAAATGGTAG |

### C. Receptors for Detecting Protein Variants

In certain embodiments, a sensor cell of the present disclosure comprises a heterologous receptor for binding to a variant of a polypeptide, e.g., a variant protein, and/or the wild type polypeptide, e.g., the wild type protein. For example, but not by way of limitation, a sensor cell of the present disclosure comprises a nucleic acid that encodes a heterologous receptor. In certain embodiments, a sensor cell can include one or more, two or more, three or more, four or more, five or more or six or more heterologous receptors.

In certain embodiments, a sensor cell can include one or more receptors that bind to one or more polypeptide variants, e.g., protein variants, and/or the wild type protein. In certain embodiments, a sensor cell can include one or more receptors that bind to one polypeptide variant, e.g., protein variant, and the wild type protein. In certain embodiments, a sensor cell can include one or more receptors that bind to a fragment of a polypeptide variant, e.g., protein variant, and a fragment of the wild type protein. In certain embodiments, a sensor cell can include a receptor that binds to two or more variants of a polypeptide (e.g., protein or fragment thereof), e.g., three or more, four or more or five or more variants of a polypeptide (e.g., protein or a fragment thereof), and/or the wild type form of the protein. In certain embodiments, a sensor cell can include a receptor that binds to two or more variants of a protein or fragment thereof, e.g., three or more, four or more or five or more variants of a protein, or the wild type form of the protein or fragment thereof. In certain embodiments, a sensor cell can include a receptor that binds to two or more variants of a protein or fragment thereof, e.g., three or more, four or more or five or more variants of a protein, and the wild type protein or fragment thereof.

In certain embodiments, a sensor cell can include one or more receptors that bind to one or more polypeptide variants, e.g., protein variants, and/or polypeptides, e.g., proteins, that are clinically relevant to an infection, disease and/or disorder. For example, but not by way of limitation, a sensor cell can include one or more receptors that bind to one or more polypeptide variants, e.g., protein variants, that are clinically relevant to a neurological disorder, a blood disorders, a cancer and a viral disease and/or infection. In certain embodiments, a sensor cell can include one or more receptors that binds to one or more peptides or fragments derived from a polypeptide or protein or interest, e.g., a polypeptide or protein or interest that is clinically relevant to an infection, disease and/or disorder. In certain embodiments, a sensor cell can include one or more receptors that binds to one or more peptides or fragments derived from a protein variant disclosed herein. In certain embodiments, a sensor call can include one or more receptors that binds to one or more peptides or fragments derived from a wild type protein disclosed herein.

In certain embodiments, the heterologous receptor binds to one or more variants of a protein from a virus that results in an infection. In certain embodiments, the heterologous receptor binds to one or more variants of a protein from a respiratory virus and/or the wild type form of the protein. Non-limiting examples of respiratory viruses include influenza viruses, respiratory syncytial virus, parainfluenza viruses, metapneumovirus, rhinovirus, coronaviruses, adenoviruses and bocaviruses. Additional non-limiting examples of respiratory viruses are disclosed in Boncristiani, Encyclopedia of Microbiology. 500-518 (2009), e.g., Table 1 of Boncristiani.

In certain embodiments, the heterologous receptor binds to a variant of a protein and/or the wild type form of the protein from a coronavirus. In certain embodiments, the coronavirus is an alpha-coronavirus, a beta-coronavirus, a gamma-coronavirus or a delta-coronavirus. In certain embodiments, the coronavirus is an alpha-coronavirus. In certain embodiments, the coronavirus is a beta-coronavirus. In certain embodiments, the coronavirus is a gamma-coronavirus. In certain embodiments, the coronavirus is a delta-coronavirus. In certain embodiments, the coronavirus is an alpha-coronavirus such as, but not limited to, the coronavirus strains HCoV-229E and HCoV-NL63. In certain embodiments, the coronavirus is a beta-coronavirus such as, but not limited to, the coronavirus strains HCoV—OC43, HCoV—HKU1, MERS-CoV (which causes Middle East Respiratory Syndrome or MERS) and SARS-CoV (which causes severe acute respiratory syndrome or SARS). In certain embodiments, the coronavirus is MERS-CoV. In certain embodiments, the coronavirus is SARS-CoV. In certain embodiments, the coronavirus is SARS-CoV-2.

In certain embodiments, the heterologous receptor binds to a wild type protein and a variant of the protein and/or multiple variants from a SARS-CoV-2 virus (which causes coronavirus disease 2019 or COVID-19). In certain embodiments, the heterologous receptor binds to a variant of a protein from a SARS-CoV-2 variant. In certain embodiments, the SARS-CoV-2 variant can be an alpha variant, a beta variant, a delta variant, a gamma variant, an epsilon variant, a kappa variant, an iota variant, an eta variant, a lambda variant, a mu variant, a zeta variant or an omicron variant. Non-limiting examples of SARS-CoV-2 variants include the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron), B.1.640.2 (ihu) and B.1.526 (iota) variants. In certain embodiments, the coronavirus is the SARS-CoV-2 B.1.1.7 variant. In certain embodiments, the SARS-CoV-2 variant is the SARS-CoV-2 B.1.351 variant. In certain embodiments, the SARS-CoV-2 variant is the SARS-CoV-2 P.1 variant. In certain embodiments, the SARS-CoV-2 variant is the SARS-CoV-2 B.1.427 variant. In certain embodiments, the SARS-CoV-2 variant is the SARS-CoV-2 B.1.429 variant. In certain embodiments, the SARS-CoV-2 variant is the SARS-CoV-2 B.1.617.2 variant.

In certain embodiments, the heterologous receptor binds to one or more variants of a protein from a coronavirus, e.g., SARS-CoV-2, and/or the wild type form of the protein. In certain embodiments, the heterologous receptor binds to one or more variants of a structural protein from a coronavirus, e.g., SARS-CoV-2. For example, but not by way of limitation, the heterologous receptor binds to one or more variants of a spike (S) protein or fragment thereof from a coronavirus, e.g., SARS-CoV-2. In certain embodiments, the S protein or fragment thereof is the receptor-binding domain (RBD) of the S protein. In certain embodiments, the heterologous receptor binds to one or more variants of the nucleocapsid protein from a coronavirus, e.g., SARS-CoV-2. In certain embodiments, the heterologous receptor binds to one or more variants of the M protein from a coronavirus, e.g., SARS-CoV-2. In certain embodiments, the heterologous receptor binds to one or more variants of the E protein from a coronavirus, e.g., SARS-CoV-2.

In certain embodiments, the heterologous receptor binds to a variant of an S protein of a coronavirus variant, e.g., a SARS-CoV-2 variant. In certain embodiments, the variant of the S protein differs by at least one amino acid modification, e.g., from about one to about ten amino acid modifications, from a wild type S protein or another variant of the S protein. In certain embodiments, the heterologous receptor also binds to the wild type S protein.

In certain embodiments, the heterologous receptor is a fungal GPCR receptor, e.g., a fungal Ste2-type or Ste3-type GPCR from Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for use in the present disclosure comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a GPCR receptor of Saccharomyces cerevisiae, Saccharomyces castelii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate.

In certain embodiments, a GPCR for use in the present disclosure comprises a GPCR receptor of Saccharomyces cerevisiae, Saccharomyces castellii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipitis, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinerea, Beauvaria bassiana, Neurospora crassa, Sporothrix scheckii, Magnaporthe oryzea, Dactylellina haptotyla, Fusarium graminearum and/or Capronia coronate that is engineered by directed evolution to bind the analyte.

In certain embodiments, a GPCR for use in the present disclosure comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence of a GPCR disclosed herein, e.g., an amino acid sequence set forth in Table 6 or Table 8. In certain embodiments, a GPCR for use in the present disclosure comprises an amino acid sequence of a GPCR disclosed herein, e.g., an amino acid sequence set forth in Table 6 or Table 8. In certain embodiments, one or more of the GPCRs provided in Table 6 or Table 8 can be engineered by directed evolution to specifically bind a variant of a protein and/or the wild type form of the protein. For example, but not by way of limitation, one or more of the GPCRs provided in Table 6 or Table 8 can be engineered by directed evolution to specifically bind a variant of an S protein of a coronavirus variant, e.g., a SARS-CoV-2 variant, and/or the wild type form of the protein.

TABLE 8

| Species | GPCR Amino Acid Sequence |
|---|---|
| Saccharomyces | MSDAAPSLSNLFYDPTYNPGQSTINYTSIYGNGSTITFDELQGLVNSTVTQA |
| cerevisiae | IMFGVRCGAAALTLIVMWMTSRSRKTPIFIINQVSLFLIILHSALYFKYLLS |
| (ScSte2) | NYSSVTYALTGFPQFISRGDVHVYGATNIIQVLLVASIETSLVFQIKVIFTG |
| | DNFKRIGLMLTSISFTLGIATVTMYFVSAVKGMIVTYNDVSATQDKYFNAST |
| | ILLASSINFMSFVLVVKLILAIRSRRFLGLKQFDSFHILLIMSCQSLLVPSI |
| | IFILAYSLKPNQGTDVLTTVATLLAVLSLPLSSMWATAANNASKTNTITSDE |
| | TTSTDRFYPGTLSSFQTDSINNDAKSSLRSRLYDLYPRRKETTSDKHSERTE |
| | VSETADDIEKNQFYQLPTPTSSKNTRIGPFADASYKEGEVEPVDMYTPDTAA |
| | DEEARKFWTEDNNNL |
| Candida albicans | MNINSTFIPDKPGDIIISYSIPGLDQPIQIPFHSLDSFQTDQAKIALVMGIT |
| (CaSte2) | IGSCSMTLIFLISIMYKTNKLTNLKLKLKLKYILQWINQKIFTKKRNDNKQQ |
| | QQQQQQQIESSSYNNTTTTLGGYKLFLFYLNSLILLIGIIRSGCYLNYNLGP |
| | LNSLSFVFTGWYDGSSFISSDVTNGFKCILYALVEISLGFQVYVMFKTSNLK |
| | IWGIMASLLSIGLGLIVVAFQINLTILSHIRESRAISTNRSEEESSSSLSSD |
| | SVGYVINSIWMDLPTILFSISINIMTILLIGKLIIAIRTRRYLGLKQFDSFH |
| | ILLIGFSQTLIIPSIILVVHYFYLSQNKDSLLQQISLLLIILMLPLSSLWAQ |
| | TANNTHNINSSPSLSFISRHHLSDSSRSGGSNTIVSNGGSNGGGGGGGNFPV |
| | SGIDAQLPPDIEKILHEDNNYKLLNSNNESVNDGDIIINDEGMITKQITIKR |
| | V |
| Schizosaccharomyces | MRQPWWKDFTIPDASAIIHQNITIVSIVGEIEVPVSTIDAYERDRLLTGMTL |
| pombe | SAQLALGVLTILMVCLLSSSEKRKHPVFVENSASIVAMCLRAILNIVTICSN |
| (SpSte2) | SYSILVNYGFILNMVHMYVHVENILILLLAPVIIFTAEMSMMIQVRIICAHD |
| | RKTQRIMTVISACLTVLVLAFWITNMCQQIQYLLWLTPLSSKTIVGYSWPYF |
| | IAKILFAFSIIFHSGVFSYKLFRAILIRKKIGQFPFGPMQCILVISCQCLIV |
| | PATFTIIDSFIHTYDGFSSMTQCLLIISLPLSSLWASSTALKLQSMKTSSAQ |
| | GETTEVSIRVDRTFDIKHTPSDDYSISDESETKKWT |
| Schizosaccharomyces | MREPWWKNYYTMNGTQVQNQSIPILSTQGYIQVPLSTIDKAERNRILTGMTV |
| octosporus | SAQLALGVLIMVMSILLSSPEKRKTPVFIVNSASIISMCIRAILMIVNLCSE |
| (SoSte2) | SYSLAVMYGFVFELVGQYVHVEDILVMIIGTIIIITAEVSMLLQVRIICAHD |
| | RKTQRIVTCISSGLSLIVVAFWFTDMCQEIKYLLWLTPYNNHQISGYYWVYF |
| | VGKILFAVSIMFHSAVFSYKLFHAIQIRKKIGQFPFGPMQCILIISCQCLFV |
| | PAIFTIIDSFIHTYDGFSSMTQCLLIVSLPLSSLWASSTALKLQSLKSTTSP |
| | GDTTQVSIRVDRTYDIKRIPTEELSSVDETEIKKWP |
| Schizosaccharomyces | MYSWDEFRSPKQAEVLNQTVTLETIVSTIQLPISEIDSMERNRLLTGMTVAV |
| japonicus | QVGLGSFILVLMCIFSSSEKRKKPVFIFNFAGNLVMTLRAIFEVIVLASNNY |
| (SjSte2) | SIAVQYGFAFAAVRQYVHAFNIIILLLGPFILFIAEMSLMLQVRIICSQHRP |
| | TMITTTVISCIFTVVTLAFWITDMSQEIAYQLFLKNYNMKQIVGYSWLYFIA |
| | KITFAASIIFHSSVFSFKLMRAIYIRRKIGQFPFGPMQCIFIVSCQCLIVPA |
| | IFTLIDSFTHTYDGESSMTQCLLIISLPLSSLWATHTAQKLQTMKDNTNPPS |
| | GTQLTIRVDRTFDMKFVSDSSDGSFTEKTEETLP |
| Baudoinia | MASNGWQNNATFDPYAQTFVLLQPDGLTPFPALLGDVLALNTVSVTQGIIYG |
| compniacensis | TQVGISGLLLLILLIMTKPDKRRSLVFILNSLSLLLIFARNVLSCVQLTTIE |
| (BcomSte2) | YNFYNWELHWYPESPALSRAMDLSAATEVLNIPIDVAIFSSLVVQVHIVCCT |
| | IHTLVRTSALLSSAAVGLAAVAVRFALAVVNIKYSIFGINTLTEPQFNLIVH |
| | LKRVSDILTVVAIAFFSSIFVAKLGVAIHTRRTLNLKNFGAIQIIFIMGCQT |
| | MLIPLIFVIVSFYASRGSQIGSMVPTVVATFLPLSGMWASAQTNNEKMGRAD |
| | QRFHRAVPVGATDFSVTKARSAKASDTLDTLIGDD |
| Candida (Clavispora) | MNPADINIEYTLGDTAFSSTFADFEAWKTRNTQFAIVNGVALACGIILMVVS |
| lusitaniae | WIIIVNKRAPIFAMNQTMLVIMVIKSAMYLKHIMGPLNSLTFRFTGLMEESW |
| (ClSte2) | APYNVYVTINVLHVLLVAAVESSLVFQIHVVEKSSRARVAGRAIVSAMSTLA |
| | LLIVSLYLYSTVRHAQTLRAELSHGDTTTVEPWVDNVPLILFSASLNVLCLL |
| | LALKLVFAVRTRRHLGLRQFDSFHILIIMATQTFVIPSSLVIANYRYASSPL |
| | LSSISIIVAVCNLPLCSLWACSNNNSSYPTSSQNTILSRYETETSQATDASS |
| | TTCAGIAEKGFDKSPDSPTFGDQDSVSISHILDSLEKDVEGVTTHRLT |
| Candida tropicalis | MDINNTIQSSGDIIITYTIPGIEEPFELPFEVLNHFQSEQSKNCLVMGVMIG |
| (CtSte2) | SCSVLLIFLVGILFKTNKESTIGKSKNLSKNFLFYLNCLITFIGIIRAACES |
| | NYLLGPLNSASFAFTGWYNGESYASSEAANGERVILFALIETSMVFQVFVMF |
| | RGAGMKKLAYSVTILCTALALVVVGFQINSAVLSHRREVNTVNEIGDTGLSS |
| | IWLDLPTILFSVSVNLMSVLLIGKLIMAIKTRRYLGLKQFDSFHVLLICSTQ |
| | TLLVPSLILFVHYFLFERNANVMLINISILLIVLMLPFSSLWAQTANTTQYI |
| | NSSPSESFISREPSANSTLHSSSGHYSEKSYGINKLNTQGSSPATLKDDHNS |
| | VILEATNPMSGEDAQLPPDIARFLQDDIRIEPSSTQDFVSTEVTYKKV |
| Saccharomyces | MSDAPPPLSELFYNSSYNPGLSIISYTSIYGNGTEVTENELQSIVNKKITEA |
| castellii | IMFGVRCGAAILTIIVMWMISKKKKTPIFIINQVSLFLILLHSAFNFRYLLS |
| (ScaSte2) | NYSSVTFALTGFPQFIHRNDVHVYAAASIFQVLLVASIEISLMFQIRVIFKG |
| | DNFKRIGTILTALSSSLGLATVAMYFVTAIKGIIATYKDVNDTQQKYENVAT |
| | ILLASSINEMTLILVIKLILAIRSRRELGLKQFDSFHILLIMSFQSLLAPSI |
| | LFILAYSLDPNQGTDVLVTVATLLVVLSLPLSSMWATAANNASRPSSVGSDW |
| | TPSNSDYYSNGPSSVKTESVKSDEKVSLRSRIYNLYPKSKSEFEQSSEHTYV |
| | DKVDLENNFYELSTPITERSPSSIIKKGKQGISTRETVKKLDSLDDIYTPNT |
| | AADEEARKFWSEDVSNELDSLQKIETETSDELSPEMLQLMIGQEEEDDNLLA |
| | TKKITVKKQ |
| Candida auris | MEFTGDIVLKYTLGGEEYLSTFEQLDSSVNRSLELGVVHGIAIACGVLLMVL |
| (CauSte2) | AWVIIIKKKNPIFVLNQLTLLLMVIKSSLYLAFLFGPLSSLTYKFTRVLPHD |
| | KWHAFHVYIATNVIHTLLIATVEMTLVFQIYIIFKSPEVRHLGYILTGAASA |
| | LALTIVALYIHSTVISAVQLKEQLLMHEIKITNSWVNNVPIILESASLNVVC |
| | IILIAKLALAIKTRRYLGLKQFDGLHILMITSTQTFIVPSVLMIVNYKQSSS |
| | YLTLLANISVILVVCNLPLSSLWAASANNSSTPTSSANTVESRWDSKESDTE |
| | TIAHELPLIPGKAEKLQLVSPITEKGDTHTMCESHGDQDLIDKMLDDIEGAV |
| | MTTEFNLNNRTV |
| Candida parapsilosis | MNKIVSKLSSSDVIVTVTIPNEEDGTYEVPFYAIDNYHYSRMENAVVLGATI |
| (CpSte2) | GACSMLLIMLIGILFKNFQRLRKSLLENINFAILLMLILRSACYINYLMNNL |
| | SSISFFFTGIFDDESFMSSDAANAFKVILVALIEVSLTYQIYVMFKTPMLKS |
| | WGIFASVLAGVLGLATLATQIYTTVMSHVNFVNGTTGSPSQVTSAWMDMPTI |
| | LESVSINVLSMFLVCKLGLAIRTRRYLGLKQFDAFHILFIMSTQTMIIPSII |
| | LFVHYFDQNDSQTTLVNISLLLVVISLPLSSLWAQTANNVRRIDTSPSMSFI |
| | SREASNRSGNETLHSGATISKYNTSNTVNTTPGTSKDDSLFILDRSIPEQRI |
| | VDTGLPKDLEKFINNDFYEDDGGMIAREVTMLKTAHNNQ |
| Kluyveromyces lactis | MSEEIPSLNPLFYNETYNPLQSVLTYSSIYGDGTEITFQQLQNLVHENITQA |
| (KlSte2) | IIFGTRIGAAGLALIIMWMVSKNRKTPIFIINQSSLVLTIVQSALYLSYLLS |
| | NFGGVPFALTLFPQMIGDRDKHLYGAVTLIQCLLVACIEVSLVFQVRVIFKA |
| | DRYRKIGIILTGVSASFGAATVAMWMITAIKSIIVVYDSPLNKVDTYYYNIA |
| | VILLACSINFITLLLSVKLFLAFRARRHLGLKQFDSFHILLIMSTQTLIGPS |
| | VLYILAYALNNKGVKSLTSIATLLVVLSLPLTSIWAAAANDAPSASTFYRQF |
| | NPYSAQNRDDSSSYSYGKAFSDKYSFSNSPQTSDGCSSKELELSTQLEMDLE |
| | SGESFMDRAKRSDFVSSPGSTDATVIKQLKASNIYTSETDADEEARAFWVNA |
| | IHENKDDGLMQSKTVEKELR |
| Fusarium | MSKEVFDPFTQNVTFFAPDGKTEISIPVAAIDQVRRMMVNTTINYATQLGAC |
| graminearum | LIMLVVLLVMVPKEKFRRPFMILQITSLVISCCRMLLLSIFHSSQFLDFYVE |
| (FgSte2) | WGDDHSRIPRSAYAPSVAGNTMSLCLVISVETMLMSQAWTMVRLWPNVWKYI |
| | IAGVSLIVSIMAISVRLAYTIIQNNAVLKLEPAFHMFWLIKWTVIMNVASIS |
| | WWCAIFNIKLVWHLISNRGILPSYKTFTPMEVLIMTNGILMIIPVIFASLEW |
| | AHFVNFESASLTLTSVAVILPLGTLAAQRIASSAPSSANSTGASSGIRYGVS |
| | GPSSFTGFKAPSFSTGTTDRPHVSIYARCEAGTSSREHINPQGVELAKLDPE |
| | TDHHVRVDRAFLQREERIRAPL |
| Komagataella (Pichia) | MEEYSDSFDPSQQLLNFTSLYGETDATFAELDDYHFYVVKYAIVYGARIGVG |
| pastoris | MFCTLMLFVVSKSWKTPIFVLNQSSLILLIIHSGFYIHYLTNQFSSLTYMFT |
| (KpSte2) | RIPNETHAGVDLRINVVTNTLYALLILSIEISLIYQVFVIFKGVYENSLRWI |
| | VTIFTALFAAAVVAINFYVTTLQSVSMYNSNVDFPRWASNVPLILFASSVNW |
| | ACLLLSLKLFFAIKVRRSLGLRQFDTFHILAIMFSQTLIIPSILIVLGYTGT |
| | RDRDSLASLGFLLIVVSLPFSSMWAATANNSNIPTSTGSFAWKNRYSPSTYS |
| | DDTTAVSKSFTIMTAKDECFTTDTEGSPRFIKGDRTSEDLHE |
| Candida tenuis | MDSYLLNHPGDISLNFALPLSDEVYTITENDLDSQSSFSIQYLVIHSCAITV |
| (CnSte2) | CLTLLVLLNLFIRNKKTPVFVLNQVILFFAIVRSSLFIGFMKSPLSTITASF |
| | TGIISDDQKHFYKVSVAANAALIILVMLIQVSFTYQIYIIFRSPEVRKFGVF |
| | MTSALGVLMAVTFGFYVNSAVASTKQYQHIFYSTDPYIMDSWVTGLPPILYS |
| | ASVIAMSLVLVLKLVAAVRTRRYLGLKQFSSYHILLIMFTQTLFVPTILTIL |
| | AYAFYGYNDILIHISTTITVVLLPFTSIWASIANNSRSLMSAASLYFSGSNS |
| | SLSELSSPSPSDNDTLNENVFAFFPDKLQKMNSSEAVSAVDKVVVHDHEDTI |
| | SQKSIPHDILEILQGNEGGQMKEHISVYSDDSFSKTTPPIVGGNLLITNTDI |
| | GMK |
| Botrytis cinerea | MASNSSNFDPLTQSITILMADGITTVSFTPLDIDFFYYYNVACCINYGAQAG |
| (BcSte2) | ACLLMFFVVVVLTKAVKRKTLLFVLNVLSLIFGELRAMLYAIYFLQGENDFY |
| | AAFTFDESRVPRSSYASSVAGSVIPLCMTITVNMSLYLQAYTVCKNLDDIKR |
| | IILTTLSAIVALLAIGERFAATVVNSVAILATSASSVPMQWLVKGTLVTETI |
| | SIWFFSLIFTGKLVWTLYNRRRNGWRQWSAVRILAAMGGCTMVIPSIFAILE |
| | YVTPVSFPEAGSIALTSVALLLPISSLWAGMVTDEETSAIDVSNLTGSRTML |
| | GSQSGNFSRKTHASDITAQSSHLDFSSRKGSNATMMRKGSNAMDQVTTIDCV |
| | VEDNQANRGLRDSTEMDLEAMGVRVNKSYGVQKA |
| Beauvaria bassiana | MDGSSAPSSPTPDPTFDRFAGNVTFFLADHITTTSVPMPVLNAYYDESLCTT |
| (BbSte2) | MNYGAQLGACLVMLVVVVALTPAAKLARRPASALHLVGLLLCAVRSGLLFAY |
| | FVSPISHFYQVWAGDESAVSRRYWDASLAANTLAFPLVVVVEAALINQAWTM |
| | VAFWPRAAKAAACACSAVIVLLTIGTRLAYTIVQNHAIVTAVPPEHELWAIQ |
| | WSAVMGAVSIFWFCAVFNVKLVCHLVANRGILPSISVVNPMEVLVMTNGTLM |
| | IIPSIFAGLEWAKFTNFESGSLTLTSVIIILPLGTLAAQRISGQGSQGYQAG |
| | HLFHEQQQQQARTRSGAFGSASQQSHPTNKVPSSITLSTSGTPITPQISAGS |
| | RPELPLVDRSERLDPIDLELGRIDAFRGSSDESPSTARPKRMQRDNFA |
| Hypocrea jecorina | MSSFDPYTQNITILVSPSSPPISIPIPVIDAFNDETASIITNYAAQLGAALA |
| (HjSte2) | MLLVLLAATPTARLLRADGPSLLHALALLVCVVRTVLLIYFFLTPFSHFYQV |
| | WTGDFSQVPAWNYRASIAGTVLSTLLTVVTDAALVNQAWTMVSLFAPRTKRA |
| | VCVLSLLITLLAISFRVAYTVIQCEGIAELAAPRQYAWLIRATLIFNICSIA |
| | WFCALFNSKLVAHLVTNRGVLPSRRAMSPMEVLIMANGILMIVPVVFAILEW |
| | HHFINFEAGSLTPTSIAIILPLSSLAAQRIANTSSS |
| Pseudogymnoascus | MSTANVHLPADFDPTRQNITIYTPDGTPVVATLPMINLENRQNNEICVVYGC |
| destructans | QLGASLIMFLVVLLTTRVSKRKSPIFVLNVLSLIISCLRSLLQILYYIGPWT |
| (PdSte2) | EIYRYLSFDYSTVPASAYANSVAATLLTLELLITIEASLVLQTNVVCKSMSS |
| | HIRWPVTALSMVVSLLAISFRFGLTIRNIEGILGATVKSDSLMFSGASLISE |
| | TASIWFFCTIFVIKLGWTLYQRKKMGLKQWGPMQIITIMAGCTMLIPSLFTV |
| | LEFFPEETFYEAGTLAICLVAILLPLSSVWAAAAIDGDEPVRPHGSTPKFAS |
| | FNMGSDYKSSSAHLPRSIRKASVPAEHLSRTSEEELGDDGTLNRGGAYGMDR |
| | MSGSISPRGVRIERTYEVHTAGRGGSIEREDIF |
| Lodderomyces | MDEAINANLVSGDIIVSFNIPGLPEPVQVPFSEFDSFHKDQLIGVIILGVTI |
| elongisporous | GACSLLLILLLGMLYKSREKYWKSLLFMLNVCILAATILRSGCFLDYYLSDL |
| (LeSte2) | ASISYTFTGVYNGTSFASSDAANVEKTIMFALIETSLTFQVYVMFQGTTWKN |
| | WGHAVTALSGLLSVASVAFQIYTTILSHNNENATISGTGTLTSGVWMDLPTL |
| | LFAASINFMTILLLFKLGMAIRQRRYLGLKQFDGFHILFIMFTQTLFIPSIL |
| | LVIHYFYQAMSGPFIINMALFLVVAFLPLSSLWAQTANTTKKIESSPSMSFI |
| | TRRKSEDESPLAANDEDRLRKFTTTLDLSGNKNNTTNNNNNSNNINNNMSNI |
| | NYPSTGLGEDDKSFIFEMEPSRERAAIEEIDLGARIDTGLPRDLEKFLVDGE |
| | DDSDDGEGMIAREVTMLKK |
| Penicillium | MATSSPIQPFDPFTQNVTFRLQDGTEFPVSVKALDVFVMYNVRVCINYGCQF |
| chrysogenum | GASFVLLVILVLLTQSDKRRSAVFILNGLALFLNSSRLLFQVIHFSTAFEQV |
| (PrSte2) | YPYVSGDYSSVPWSAYAISIVAVVLTTLVVVCIEASLVIQVHVVCSTLRRRY |
| | RHPLLAISILVALVPIGERCAWMVANCKAIIKLTYTNDVWWIESATNICVTI |
| | SICFFCVIFVTKLGFAIKQRRRLGVREFGPMKVIFVMGCQTMVVPAIFSITQ |
| | YYVVVPEFSSNVVTLVVISLPLSSIWAGAVLENARRTGSQDRQRRRNLWRAL |
| | VGGAESLLSPTKDSPTSLSAMTAAQTLCYSDHTMSKGSPTSRDTDAFYGISV |
| | EHDISINRVQRNNSIV |
| Paracoccidioides | MAPSFDPFNQNVVFHKADGTPENVSIHELDDFVQYNTRVCINYSSQLGASVI |
| brasiliensis | AGLMLAMLTHSEKRRLPVFFLNTFALAMNFARLLCMTIYFTTGENKSYAYFG |
| (PbSte2) | QDYSQVPGSAYAASVLGVVFTTLLVISMEMSLLIQTRVVCTTLPDIQRYLLM |
| | AVSSAISLMAIGFRLGLMVENCIAIVQASNFAPFIWLQSASNITITISTCFF |
| | SAVFVTKLAYALVTRIRLGLTRFGAMQVMFIMSCQTMVIPAIFSILQYPLPK |
| | YEMNSNLFTLVAIFLPLSSLWASVATKSSFETSSSGRHQYLWPSEQSNNVTN |
| | SEIKYQVSFSQNHTTLRSGGSVATTLSPDRLDPVYSEVEAGTKA |
| Candida (Pichia) | MKSCSIGFGIPFINEPNFETVSILTMDVSFIDADVNPDNILLNFTIPGYQNG |
| guilliermondii | FSVPMVVINELQKSQMKYAIVYGCGVGASLILLFVVWILCSRKTPLFIMNNI |
| (CguSte2) | PLVLYVISSSLNLAYITGPLSSVSVELTGILTSHDAINVVYASNALQMLLIF |
| | SIQSTMAYHVYVMFKSPQIKYLRYMLVGFLGCLQIVTTCLYINYNVLYSRRM |
| | HKLYETGQTYQDGTVMTFVPFILFQCSVNFSSIFLVLKLIMAIRTRRYLGLR |
| | QFGGFHILMIVSLQTMLVPSILVLVNYAAHKAVPSNLLSSVSMMIIVLSLPA |
| | SSMWAAAANASSAPSSAASSLFRYTTSDSDRTLETKSDHFIMKHESHNSSPN |
| | SSPLTLVQKRISDATLELPKELEDLIDSTSI |
| Zygosaccharomyces | MSEINNSTYNPMNAYVTFTSIYGDDTMVRFKDVELVVNKRVTEAIMFGVKVG |
| rouxii | AASLTLIIMWMISKKRTTPIFIINQSSLVFTIIHASLYFGYLLSGFGSIVYN |
| (ZrSte2) | MTSFPQLISSNDVRVYAATNIFEVLLVASIEISLVFQVKVMFANNNGRRWTW |
| | CLMVVSIGMALATVGLYFATAVELIRAAYSNDTVSRHVFYNVSLILLASSVN |
| | LMTLMLVVKLVLAIRSRRELGLKQFDSFHILLIMSCQTLIAPSILFILGWTL |
| | DPHTGNEVLITVGQLLIVLSLPLSSMWATTANNTSSSSSSVSCNDSSFGNDN |
| | LCSKSSQFRRTFMNRFRPKSVNGDGNSENTFVTIDDLEKSVFQELSTPVSGE |
| | SKIDHDHASSISCQKTCNHVHASTVNSDKGSWSSDGSCGSSPLRKTSTVNSE |
| | DLPPHILSAYDDDRGIVESKKIILKKL |
| Geotrichum candidum | MAEDSIFPNNSTSPLTNPIVVETIKGTAYIPLHYLDDLQYEKMLLASLESVR |
| (GcSte2) | IATSFVVIIWYFVAVNKAKRSKFLYIVNQVSLLIVFIQSILSLIYVESNESK |
| | MSTILTGDYTGITKRDINVSCVASVFQFLFIACIELALFIQATVVFQKSVRW |
| | LKFSVSLIQGSVALTTTALYMAIIVQSIYATLNPYAGNLIKGREGYLLASLG |
| | KIFFSISVTSCMCIFVGKLVFAIHQRRTLGIKQFDGLQILVIMSTQSMIIPT |
| | IIVLMSFLRRNAGSVYTMATLLVALSLPLSSLWAEAKTTRDSASYTAYRPSG |
| | SPNNRSLFAIFSDRLACGSGRNNRHDDDSRGNGSVNARKADVESTIEMSSCY |
| | TDSPTYSKFEAGLDARGIVFYNEHGLPVVSGEVGGSSSNGTKLGSGHKYEVN |
| | TTVVLSDVDSPSPTDVTRK |
| Neurospora crassa | MASSSSPPADIFSGITQSLNSTHATLTLPIPPADRDHLENQVLFLEDNHGQL |
| (NcSte2) | LNVTTTYIDAFNNMLVSTTINYATQIGATFIMLAIMLLMTPRRRFKRLPTII |
| | SLLALCINLIRVVLLALFFPSHWTDFYVLYSGDWQFVPPGDMQISVAATVLS |
| | IPVTALLLSALMVQAWSMMQLWTPLWRALVVLVSGLLSLVTVAMSFANCIFQ |
| | AKNILYADPLPSYWVRKLYLALTTGSISWFTFLFMIRLVMHMWTNRSILPSM |
| | KGLKAMDVLIITNSILMLIPVLFAGLEFLDSASGFESGSLTQTSVVIVLPLG |
| | TLVAQRIATRGYMPDSLEASSGPNGSLPLSNLSFAGGGGGGSGGHKDKENGG |
| | GIIPPTTNNTAATNESSSIACSGISCLPKVKRMTASSASSSQRPLLTMINST |
| | IASNDSSGFPSPGIHNTTTTTTQYQYSMGMNMPNFPPVPFPGYQSRTTGVTS |
| | HIVSDGRHHQGMNRHPSVDHFDRELARIDDEDDDGYPFASSEKAVMHGDDDD |
| | DVERGRRRALPPSLGGVRVERTIETRSEERMPSPDPLGVTKPRSFE |
| Vanderwaltozyma | MSGIDDMGDKPDILGLFYDANYDPGQGILTFISMYGNTTITEDELQLEVNSL |
| polyspora 2 | ITSGIMFGVRCGAACLTLLIMWMISKNKKTPIFIINQCSLILIIMHSGLYFK |
| (Vp2Ste2) | NILSNLNSLSYILTGFTQNITKNNIHVEGAANIIQVLLVATIELSLVFQIRV |
| | MFKGDSFRKAGYGLLSIASGLGIATVVMYFYSAITNMIAVYNQTYNSTAKLE |
| | NVANILLSTSINFMTVVLIVKLFLAVRSRRYLGLKQFDSFHILLIMSCQTLI |
| | VPSILFILSYALSTKLYTDHLVVIATLLVVLSLPLSSMWASAANNSPKPSSF |
| | TTDYSNKNPSDTPSFYSQSISSSMKSKFPSKFIPFNFKSKDNSSDTRSENTY |
| | IGNYDMEKNGSPNHSYSSKDQSEVYTIGVSSMHTDIKSQKNISGQHLYTPST |
| | EIDEEARDFWAGRAVNNSVPNDYQPSELPASILEELNSLDENNEGFLETKRI |
| | TERKQ |
| Scheffersomyces | MDTSINTLNPANIIVNYTLPNDPRVISVPFGAFDEYVNQSMQKAIIHGVSIG |
| stipitis | SCTIMLLIILIFNVKRKKSPAFYLNSVTLTAMIIRSALNLAYLLGPLAGLSF |
| (SsSte2) | TFSGLVTPETNFSVSEATNAFQVIVVALIEASMTFQVFVVFQSPEVKKLGIA |
| | LTSISAFTGAAAVGFTINSTIQQSRIYHSVVNGTPTPTVATWSWVRDVPTIL |
| | FSTSVNIMSFILILKLGFAIKTRRYLGLRQFGSLHILLMMATQTLLAPSILI |
| | LVHYGYGTSLNSQLILISYLLVVLSLPVSSIWAATANNSPQLPSSATLSEMN |
| | KTTSHESES |
| Candida glabrata | MEMGYDPRMYNPRNEYLNFTSVYDVNDTIRFSTLDAIVKGLLRIAIVHGVRL |
| (CgSte2) | GAIFMTLIIMFISSNTWKKPIFIINMVSLMLVMIHSALSFHYLLSNYSSISY |
| | ILTGFPQLITSNNKRIQDAASIVQVLLVAAIEASLVFQIHVMFTIENIKLIR |
| | EIVLSISIAMGLATVATYLAAAIKLIRGLHDEVMPQTHLIENLSIILLASSI |
| | NEMTFILVIKLFFAIRSRRYLGLRQFDAFHILLIMFCQSLLIPSVLYIIVYA |
| | VDSRSNQDYLIPIANLFVVLSLPLSSIWANTSNNSSRSPKYWKNSQTNKSNG |
| | SFVSSISVNSDSQNPLYKKIVRFTSKGDTTRSIVSDSTLAEVGKYSMQDVSN |
| | SNFECRDLDFEKVKHTCENFGRISETYSELSTLDTTALNETRLFWKQQSQCD |
| | K |
| Vanderwaltozyma | MSSQSHPPLIDLFYDSSYDPGESLIYYTSIYGNNTYITFDELQTIVNKKVTQ |
| polyspora 1 | GILFGVRCGAAFLMLVAMWLISKNKRSRIFITNQCCLVEMIMHSGLYFRYLL |
| (VplSte2) | SRYGSVTFILTGFQQLLTRNDIHIYGATDFIQVALVACIELSLIFQIKVIFA |
| | GTNYGKLANYFITLGSLLGLATFGMYMLTAINGTIKLYNNEYDPNQRKYENI |
| | STILLASSINMLTLILILKLVAAIRTRRYLGLKQFDSFHILLIMSTQTLIIP |
| | SILFILSYSLREDMHTDQLIIIGNLIVVLSLPLSSMWASSLNNSSKPTSINT |
| | DESGPKSSEEGTAISLLSQNMEPSIVTKYTRRSPGLYPVSVGTPIEKEASYT |
| | LFEATDIDFESSSNDITRTS |
| Zygosaccharomyces | MSGLANNTSYNPLESFIIFTSVYGGDTMVKFEDLQLVFTKRITEGILFGVKV |
| bailii | GAASLTMIVMWMISRRRTSPIFIMNQLSLVFTILHASFYFKYLLDGEGSIVY |
| (ZbSte2) | TLTLFPQLITSSDLHVFATANVVEVLLVSSIEASLVFQVNVMFAGSNHRKFA |
| | WLLVGFSLGLALATVALYFVTAVKMIASAYASQPPTNPIYENVSLFLLAASV |
| | FLMTLMLTVKLILAIRSRRFLGLKQFDSFHILLIMSCQTLIAPSVLYILGFI |
| | LDHRKGNDYLITVAQLLVVLSLPLSSMWATTANDASSGTSMSSKESVYGSDS |
| | LYSKSKCSQFTRTFMNRFSTKPTKNDEISDSAFVAVDSLEKNAPQGISEHVC |
| | EFPQSDLSDQATSISSRKKEAVVYASTVDEDKGSFSSDINGYTVTNMPLASA |
| | ASANCENSPCHVPRPYEENEGVVETRKIILKKNVKW |

In certain embodiments, a GPCR detecting the presence of a protein variant in a sample comprises an amino acid sequence that is encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence of a GPCR disclosed herein, e.g., a nucleotide sequence set forth in Table 7. In certain embodiments, a GPCR for detecting the presence of a protein variant in a sample comprises an amino acid sequence that is encoded by nucleotide sequence of a GPCR disclosed herein, e.g., a nucleotide sequence set forth in Table 7. For example, but not by way of limitation, one or more of the GPCRs encoded by the nucleotide sequences provided in Table 7 can be engineered by directed evolution to specifically bind a variant of an S protein of a coronavirus variant, e.g., a SARS-CoV-2 variant.

### V. Proteases

In certain embodiments, a sensor cell of the present disclosure comprises a protease. In certain embodiments, a sensor cell of the present disclosure can express one or more proteases. In certain embodiments, a sensor cell can express two or more proteases, e.g., three or more, four or more, five or more or six or more proteases.

In certain embodiments, a sensor cell of the present disclosure comprises a nucleic acid that encodes a protease. In certain embodiments, the protease can be operably linked to a constitutively active promoter. Non-limiting examples of constitutively active promoters include pTef1, pGPD and pCCW12. In certain embodiments, the protease can be operably linked to an inducible promoter, e.g., peptide-inducible promoter, e.g., inducible by natural or synthetic peptides) or inducibly secreted via peptide/GPCR activation of the pheromone mating pathway.

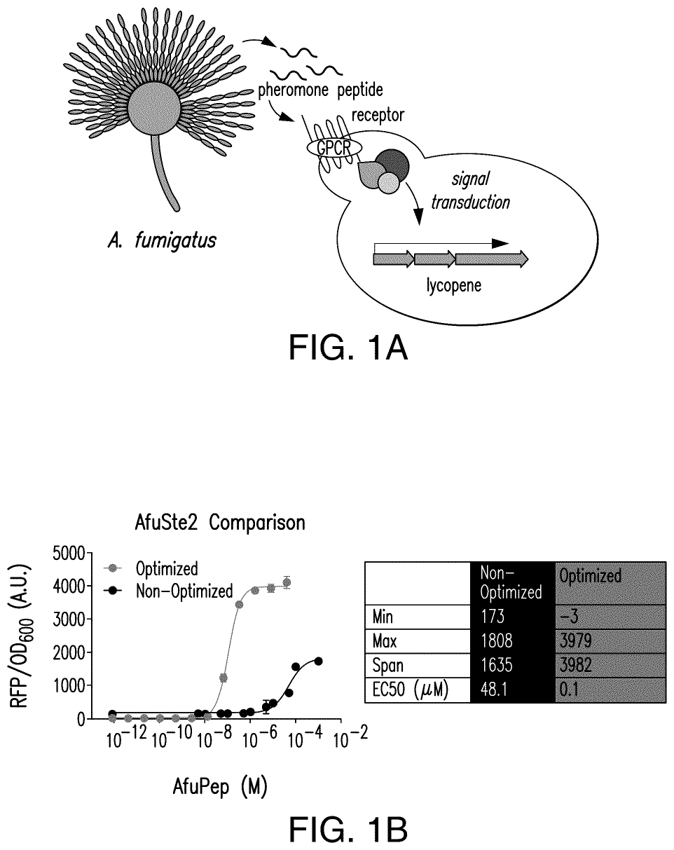

In certain embodiments, the protease can be secreted from the sensor cell. In certain embodiments, secretion can be performed using the conserved secretory pathway in fungal cells, e.g., yeast. For example, but not by way of limitation, the protease is secretable because it is coupled to a secretion signal sequence. Examples of secretion signal sequences can be obtained from proteins including mating factor alpha-1, alpha factor K, alpha factor T, glycoamylase, inulinase, invertase, lysozyme, serum albumin, alpha-amylase and killer protein. In certain embodiments, the secretion signal sequence is a secretion signal sequence obtained from a yeast protein, such as a Saccharomyces cerevisiae protein. In certain embodiments, the secretion signal peptide is obtained from the Saccharomyces cerevisiae mating factor alpha-1 (MFα1) or Kluyveromyces lactis mating factor alpha (MFα). See FIGS. 1 and 2. Additionally, mutations, substitutions and truncations of any signal peptide are also within the scope of the present disclosure. The selection and design, including additional mutations and truncations of a signal peptide is within the ability and discretion of one of ordinary skill in the art. In certain embodiments, the one or more secretion signal sequences are located at the N-terminus of a secretable peptide. In certain embodiments, a Kex2 processing site and/or a Ste13 processing site or a homolog thereof can be present between the amino acid sequence of the secretion signal sequence and the secretable analyte. Additional non-limiting examples of secretion signals are disclosed in U.S. Pat. No. 10,725,036, the contents of which is disclosed herein in its entirety. In certain embodiments, the protease can be secreted from the sensor cell using the mating secretory pathway, e.g., the alpha-mating factor secretion pathway of the sensor cell.

In certain embodiments, the protease and/or proteolytic agent is added to the sample to be analyzed before, during and/or after contacting the sample with a sensor cell.

In certain embodiments, the protease is an endoprotease, which cleaves internal peptide bonds in a polypeptide sequence. Non-limiting examples of endoproteases include serine endoproteases, aspartic endoproteases, cysteine/thiol endoproteases, metalloendoproteases and glutamic acid and threonine endoproteases. In certain embodiments, the protease is an exoprotease, which cleaves at or near the ends of polypeptides. Non-limiting examples of exoproteases include aminopeptidases and carboxypeptidases. Additional examples of proteases for use in the present disclosure are provided in Ward, Comprehensive Biology, 604-615 (2011); and Lopez-Otin and Bond, J. Biol. Chem, 283(45):30433-30437 (2008), the contents of which are incorporated herein in their entireties.

In certain embodiments, a protease for use in the present disclosure includes an enzyme of class EC 3.4, e.g., class EC 3.4.23.35. In certain embodiments, a protease for use in the present disclosure can be a protease from the Barrierpepsin (Bar) family of proteases, e.g., Bar1 of a fungal species disclosed herein. In certain embodiments, a protease from the Bar family of proteases can be engineered by directed evolution to specifically cleave a polypeptide variant.

In certain embodiments, a protease for use in the present disclosure can include Arg-C proteinase, Asp-N Endopeptidase, Caspase 1, Caspase 2, Caspase 3, Caspase 4, Caspase 5, Caspase 6, Caspase 7, Caspase 8, Caspase 9, Caspase 10, Chymotrypsin, Clostripain (also referred to as Clostridiopeptidase B), Enterokinase, Coagulation factor Xa, Glutamyl endopeptidase, Granzyme B, LysC endopeptidase, Lysyl endopeptidase, Achromobacter proteinase I, Peptidyl-Lys metalloendopeptidase (LysN), Neutrophil elastase, Pepsin, Proline-endopeptidase, Proteinase K, Staphylococcal peptidase I, Tobacco etch virus protease, Thermolysin, Thrombin and/or Trypsin.

In certain embodiments, a proteolytic agent can be used as the protease. Non-limiting examples of proteolytic agents include formic acid, NTCB (2-nitro-5-thiocyanobenzoic acid), BNPS-skatole and cyanogen bromide (CNBr).

In certain embodiments, a protease for use in the present disclosure comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in Table 9 or provided in the examples. In certain embodiments, a protease for use in the present disclosure comprises an amino acid sequence set forth in Table 9 or provided in the examples.

In certain embodiments, the specificity and/or activity of a protease and/or proteolytic agent for use in the present disclosure can be altered by exposing the protease and/or proteolytic agent to varying conditions (e.g., by modification of the conditions of the sample). For example, but not by way of limitation, the specificity and/or activity of a protease and/or proteolytic agent for use in the present disclosure can be altered by bringing the sample to a certain pH. In certain embodiments, the specificity and/or activity of a protease and/or proteolytic agent for use in the present disclosure can be altered by introducing competitors into the sample.

In certain embodiments, a sensor cell for detecting a fungal species disclosed herein can be genetically engineered to express a protease. For example, but not by way of limitation, a sensor cell for detecting a species of the Aspergillus genus can be genetically engineered to express a protease.

In certain embodiments, a sensor cell for detecting a virus disclosed herein can be genetically engineered to express a protease. For example, but not by way of limitation, a sensor cell for detecting a respiratory virus, a hemorrhagic virus, a gastrointestinal virus, an exanthematous virus, a hepatitis virus, a sexually transmitted virus and/or a neurological virus can be genetically engineered to express a protease. In certain embodiments, a sensor cell for detecting a coronavirus, e.g., SARS-CoV-2, or an ebolavirus can be genetically engineered to express a protease.

In certain embodiments, a sensor cell can be genetically engineered to express a protease to degrade and/or digest a protein comprising the analyte of interest. For example, but not by way of limitation, a protease can be used to expose an analyte, e.g., a peptide epitope, present in a full-length protein.

In certain embodiments, a sensor cell can be genetically engineered to express a protease to prevent and/or reduce self-activation. For example, but not by way of limitation, if a sensor cell expresses the analyte that binds to the receptor expressed by the sensor cell, the sensor can be genetically engineered to express a protease to degrade the basal levels of the analyte expressed by the sensor cell when not activated, as described in Example 5. In certain embodiments, a sensor cell for detecting a species of the Aspergillus genus (e.g., A. fumigatus) can be genetically modified to express a receptor and to express the analyte of the species (e.g., AfuPep) upon activation, and can be further genetically modified to express a protease (e.g., Bar1) to degrade the basal expression of the analyte to prevent self-activation of the sensor cell in the absence of exogenous analyte. In certain embodiments, the protease can be engineered by directed evolution to cleave the analyte.

In certain embodiments, a sensor cell for detecting a protein variant can be genetically engineered to express a protease. For example, but not by way of limitation, a sensor cell of the present disclosure can express a protease that specifically cleaves a variant of a polypeptide, e.g., protein. In certain embodiments, the protease can specifically cleave a variant of a polypeptide that differs by at least one amino acid from the wild type polypeptide, e.g., differs by at least two amino acid, at least three amino acids or more. In certain embodiments, the protease can specifically cleave a wild type polypeptide that differs by at least one amino acid from another polypeptide, e.g., variant of the polypeptide, e.g., differs by at least two amino acid, at least three amino acids or more. In certain embodiments, the protease can cleave the wild type polypeptide instead of a variant of the polypeptide.

In certain embodiments, a sensor cell can express one or more proteases, e.g., two or more, three or more, four or more, five or more or six or more proteases.

In certain embodiments, the sensor cell, e.g., a first sensor cell, can express a first protease that specifically cleaves a first variant of a protein.

In certain embodiments, the sensor cell, e.g., a second sensor cell, can express a first protease that specifically cleaves the wild type protein.

In certain embodiments, the sensor cell, e.g., a third sensor cell, can express a first protease that specifically cleaves a first variant of a protein and can express a second protease that specifically cleaves a second variant of the protein. In certain embodiments, the sensor cell, e.g., a third sensor cell, can include a third protease that cleaves the wild type protein.

In certain embodiments, the sensor cell can express a protease for each known variant of the protein. In certain embodiments, the sensor cell can express a protease for each known variant of the protein except for one variant. In certain embodiments, the sensor cell can further express a protease specific for the wild type protein. For example, but not by way of limitation, if there are 4 known variants and one wild type protein, a sensor cell of the present disclosure can express a first protease for cleaving the wild type protein, a second protease for cleaving the first protein variant, a third protease for cleaving the second protein variant and a fourth protease for cleaving the third protein variant so that if a sample containing the fourth protein is present in the sample, it is able to activate the receptor expressed by the sensor cell.

In certain embodiments, the protease expressed by a sensor cell disclosed herein cleaves and digests the wild type polypeptide (or fragment thereof), so it is unable to bind and activate the receptor. In certain embodiments, the protease expressed by a sensor cell disclosed herein cleaves and digests the variant of the protein (or fragment thereof), so it is unable to bind and activate the receptor.

In certain embodiments, one or more of the proteases provided in Table 9 or the examples can be engineered by directed evolution to specifically cleave a variant of a protein and/or wild type protein. For example, but not by way of limitation, one or more of the proteases provided in Table 9 or in the examples can be engineered by directed evolution to specifically cleave a variant of an S protein of a coronavirus variant, e.g., a SARS-CoV-2 variant.

TABLE 9

| Species | |
|---|---|
| (protease) | Amino acid sequence |
| Saccharomyces | MSAINHLCLKLILASFAIINTITALTNDGTGHLEFLLQHEEEMYYATTLD |
| cerevisiae | IGTPSQSLTVLFDTGSADFWVMDSSNPFCLPNSNTSSYSNATYNGEEVKP |
| (ScBar1) | SIDCRSMSTYNEHRSSTYQYLENGRFYITYADGTFADGSWGTETVSINGI |
| | DIPNIQFGVAKYATTPVSGVLGIGFPRRESVKGYEGAPNEYYPNFPQILK |
| | SEKIIDVVAYSLFLNSPDSGTGSIVFGAIDESKFSGDLFTFPMVNEYPTI |
| | VDAPATLAMTIQGLGAQNKSSCEHETFTTTKYPVLLDSGTSLLNAPKVIA |
| | DKMASFVNASYSEEEGIYILDCPVSVGDVEYNFDFGDLQISVPLSSLILS |
| | PETEGSYCGFAVQPTNDSMVLGDVFLSSAYVVEDLDNYKISLAQANWNAS |
| | EVSKKLVNIQTDGSISGAKIATAEPWSTNEPFTVTSDIYSSTGCKSRPFL |
| | QSSTASSLIAETNVQSRNCSTKMPGTRSTTVLSKPTQNSAMHQSTGAVTQ |
| | TSNETKLELSSTMANSGSVSLPTSNSIDKEFEHSKSQTTSDPSVAEHSTF |
| | NQTFVHETKYRPTHKTVITETVTKYSTVLINVCKPTY |
| Candida albicans | MLFTILSLLVPSLAVAVSNTGGAVKLDLTISDEHMYYIENLALGTPPQFI |
| (CaBar1) | SNVIVDSGSSDLMIVDSIYNFSASSSFYNSNQTAIMKYGYGGQFPVYFIN |
| | ETIRSNDWKLSNLSMGLANISDMDSFSGILGIGFTRQELFKTNYSNFPYL |
| | LKDQGYTKSVLFSFNGQDQNPSIIFGGIITNIIDGPLVRAPFIKVISFIN |
| | QLDYWLMPTFTVNQIKLGDTIVSNQKTLYQIDSGTNGFVPPTPVLNNILK |
| | ILGDDYIQDDNGNIYFDIKYIEGLNITFSVQGYDIGFQLVDIVGDTIERN |
| | STTFVALNVASCDIGYNAYEGLLPNLIFKYHYAIFDYDNAQIYFGKYKNS |
| | NGEANVVAVENGYQLPVPTVDVPDVEDTYSVIYIAESETTVTVAPTEVQS |
| | TYSSFSISEGTSSNLTSGSTSDDKITSVTSNPQYC |
| Schizosaccharomyces | MLSLFLKSLFAIIIIELTIIHALPTYTVHWKCSIQQANTSSASSNQTVQP |
| pombe | RQHAAPSSDRIKSLPEFKGSLPELYSGYLEANSDKSLFYTYAPAVVDSET |
| (SpSxa2) | FIVWLQGGPGCAGTLGFFSENGPIEISQSSPSPSLNPESWTNFANMLWLD |
| | QPFGTGYSQGQAAYTTTIEEASSDFVNALKSFYQKFPHLMKKKLYLVGES |
| | YGSIWSANFAEALLSEPSLNINFMGVGIVSGLTADYETQEQITASIWVEH |
| | ISKLGYYFNNTSSTISEEFKKRNKECQYDSVLNRLTFPTEQYPIWRPEYN |
| | FSTSTSLRKREALDGEDIGNVENSISGCDLYSLSNFLLYLENSCVITYDV |
| | SLDCSFNEYNDPLITYLNREDVRSSLHATKASTALTSGEGVFADGCNEDL |
| | YKKIVSNNVESVLVEIIPRLTEKYKVSFLAGALDLQILWTGTLLALQNTT |
| | WNGWQGFTQSPGSLETTNGFTLDERNLAFTLSNSVGHMAPSKDPQMVREW |
| | LENTLLY |
| Schizosaccharomyces | MNLAVHVLLLSFLLYTVHSFPAYPPYPIYPRSSLNSSNASSVPIPQPSYN |
| octosporus | SSSKAMNPDLVTSLPEYSGSLPELYSGYLSAENFKQIFYTFAPAITPTDK |
| (SoSxa2) | FIVWLQGGPGCAGTLGFFSENGPIILDPNHQTPQDNPNSWTRFANMMWID |
| | QPFGTGYSQGSPLASTVSIATSDFIQTLKSFYAKFPEMKEKELYLVGESY |
| | GSVWGAYFAQALRSDPELHAIPFGGLGIISGLLSDYETQNTITASMWLDH |
| | VNKMGLYYTNESAEVSTFFAAKNQQCGYAEVLERMQFPAEQTPIPKPRVN |
| | LTNSFSKKKRQADSDSDSGNIKEAFQMITDCDTYTLTDFLLYLKNHCMIT |
| | YDITLDCSFNSNNDPLTTYLNRKDVQKQLHAIHVESALTSSKGIFGNGCN |
| | YDVYGDIVKNGAPSILEEAIPELVQNYKVSFLAGALDFQLLWTGSLLALQ |
| | NTTWNGWQGFQEIPDLNSPFGFTLDERNLAFTLSNKVGHMAPSKDPSMVI |
| | DWLQKTLLG |
| Schizosaccharomyces | MVVLHRLLLSLWPAGAMLLVSSFLLNLPTTSAIPIYSDPHQCSLRVSVRT |
| japonicus | DVEGEQQQTQAMGSVVIASNVTSSDAPTENPTSSAPTTFSHTVNSTFPTH |
| (SjSxa2) | TPTTSTSTNTPTDARVSSLPLLNGDMPALYAGYLNVSASSTSPHSIFYAY |
| | APSEQGSDDLIVWLQGGPGCAGTIGLFAENGPVRFGPGDSAPVRSEHSWT |
| | RYADVLYLDQPFGTGFSYAVTEADYTTTMDAASAEFIAFLDGFFDAYPDT |
| | ASKRLFFVGESYGSFWGAYFARALADRPDIYDRIVSVGIVSGLSTTHDTQ |
| | YSVSNSLWLSHLNKLGYYTQDKSTWNSFSRVNARCGYDKVMQEFTFPASQ |
| | TPLSSISTSTSSSSTSSGAGAGATVSSLTDSSGNASYTNSSSSAEVDVES |
| | YFPSCDTNDATYMLMYLQNTCLISYDVSIDCTTASQLTDHLTPYLNRADV |
| | REALHVPSSVAAFSTANGVFGDGCNYDLLQKINTVTYEPVLKSVVPSLIA |
| | DGKRVAFLSGALDYQIPWSGTLLAMQNTTWAGWQGFQTEPDWDNPVGFTL |
| | NERGLSFTLLNSVGHMASMNDPDRVNRWLQEQVL |

### VI. Reporter

The presently disclosed sensor cells further include a reporter gene, e.g., a nucleic acid encoding a reporter. In certain non-limiting embodiments, the sensor cell comprises a receptor that binds to an analyte, wherein the receptor is coupled to a reporter gene such that when the analyte binds to the receptor, expression of the reporter gene is altered, e.g., increased or decreased.

In certain embodiments, the receptor is coupled to a detectable reporter gene such that when an analyte binds to the receptor, expression of the reporter gene is increased.

In certain embodiments, the receptor is coupled to a detectable reporter gene such that when an analyte binds to the receptor, expression of the reporter gene is inhibited (for example, by binding of a transcriptional repressor).

In certain embodiments, the reporter gene encodes a fluorescent reporter. Non-limiting examples of a fluorescent reporter include the fluorescent proteins GFP, sfGFP, deGFP, eGFP, yEGFP, Venus, ymVenus, ymTagBFP2, yIFP1.4, YFP, Cerulean, Citrine, ymTurquoise2, ymNeonGreen, CFP, eYFP, eCFP, RFP, mRFP, ytdTomato, mCherry and mmCherry. In certain embodiments, the reporter gene does not encode a fluorescent reporter. In certain embodiments, the reporter is not a fluorescent reporter, e.g., a fluorescent protein.

In certain embodiments, the reporter gene encodes a reporter detectable without instrumentation. For example, but not by way of limitation, the reporter is a biosynthesized visible-light pigment. Use of such a reporter as a simple visual readout has a number of advantages. Use of a biosynthesized visible-light pigment readout requires no complex equipment since it can be seen by the naked eye and requires no expensive externally added reagent, since it can be biosynthesized from endogenous substrates. In contrast, most whole-cell biosensors reported in the literature use laboratory readouts such as fluorescent proteins, lacZ or luciferase, which require the use of expensive equipment, externally added chromogenic reagents or both.

In certain embodiments, the reporter can be a carotenoid. Carotenoids are a class of terpenoids composed of 8 isoprene units totaling 40 carbon atoms. Lycopene is a specific naturally produced carotenoid pigment whose heterologous expression in E. coli using the genes CrtE, CrtB and CrtI. Lycopene can be visualized by the naked eye, is widely validated in yeast metabolic engineering and is non-toxic. Lycopene is the first intermediate in carotenoid biosynthesis that has a sufficiently conjugated n-system to absorb in the visible region. Thus, unlike standard laboratory reporters like lacZ that require exogenously added caged dyes (X-gal) or fluorescent proteins that require specialized equipment (fluorimeter), lycopene can be directly observed by a non-technical person.

In certain embodiments, a sensor cell of the present disclosure is genetically engineered to include one or more, two or more or all three of the genes CrtE, CrtB and CrtI. In certain embodiments, a sensor cell is genetically engineered to include CrtE and CrtB. In certain embodiments, a sensor cell is genetically engineered to include all three of genes CrtE, CrtB and CrtI. In certain embodiments, a sensor cell can include one or more, two or more or three or more copies of CrtE. In certain embodiments, a sensor cell can include one or more, two or more or three or more copies of CrtB. In certain embodiments, a sensor cell can include one or more, two or more or three or more copies of CrtI. In certain embodiments, a sensor cell can include two copies of CrtI. In certain embodiments, a sensor cell can include three copies of CrtI. In certain embodiments, a sensor cell of the present disclosure is genetically engineered to include a copy of CrtE (e.g., operably linked to a constitutively active promoter), a copy of CrtB (e.g., operably linked to a constitutively active promoter) and two copies of CrtI (e.g., operably linked to an inducible promoter). In certain embodiments, a sensor cell of the present disclosure is genetically engineered to include a copy of CrtE (e.g., operably linked to a constitutively active promoter), a copy of CrtB (e.g., operably linked to a constitutively active promoter) and three copies of CrtI (e.g., operably linked to an inducible promoter). In certain embodiments, the sensor cell can be further genetically engineered to express at least one copy of FAD (e.g., at least two or at least three copies), e.g., operably linked to a constitutively active promoter. In certain embodiments, a sensor cell of the present disclosure is genetically engineered to include a copy of CrtE (e.g., operably linked to a constitutively active promoter), a copy of CrtB (e.g., operably linked to a constitutively active promoter), two or three copies (e.g., three copies) of CrtI (e.g., operably linked to an inducible promoter) and at least one copy of FAD (e.g., operably linked to a constitutively active promoter).

Additional biosynthesized visible-light pigments include mutants of CrtI disclosed in Schmidt-Dannert, C., Umeno, D. & Arnold, F. H. Molecular breeding of carotenoid biosynthetic pathways. Nat Biotech 18, 750-753 (2000), biosynthetic enzymes that generate alternate carotenoid pigments disclosed in Umeno, D. & Arnold, F. H. Evolution of a Pathway to Novel Long-Chain Carotenoids. J. Bacteriol. 186, 1531-1536 (2004), and lycopene enzymes from alternate organism disclosed in Verwaal, R et al. High-Level Production of Beta-Carotene in Saccharomyces cerevisiae by Successive Transformation with Carotenogenic Genes from Xanthophyllomyces dendrorhous. Appl. Environ. Microbiol. 73, 4342-4350 (2007).

In certain embodiments, alternative visible pigments can be used as a reporter. For example, but not by way of limitation, violacein can be used. In certain embodiments, a sensor cell of the present disclosure is genetically engineered to include one or more enzymes involved in the biosynthetic pathway of violacein. In certain embodiments, a sensor cell of the present disclosure is genetically engineered to include one or more, two or more, three or more, four or more or all five of the genes VioA, VioB, VioE, VioD and VioC. In certain embodiments, a sensor cell of the present disclosure is genetically engineered to include all five of the genes VioA, VioB, VioE, VioD and VioC.

In certain embodiments, indigoidine can be used. In certain embodiments, a sensor cell of the present disclosure is genetically engineered to include one or more enzymes involved in the biosynthetic pathway of indigoidine. In certain embodiments, a sensor cell of the present disclosure is genetically engineered to include one or more or both of the genes idgS and sfp. In certain embodiments, a sensor cell of the present disclosure is genetically engineered to include both genes idgS and sfp.

In certain embodiments, the reporter can be a chromogenic reporter such as LacZ. In certain embodiments, the reporter is not LacZ.

In certain embodiments, a presently disclosed sensor cell can be engineered to contain the genes required for synthesis of lycopene and at least one of the genes can be the detectable reporter gene coupled to activation by peptide receptor binding (e.g., at least a portion of the Fus1 promoter). In certain embodiments, the gene activated by peptide binding to the receptor can be CrtI, CrtE and/or CrtB. In certain embodiments, the gene activated by peptide binding to the receptor is CrtI. In certain non-limiting embodiments, receptor activation induces reporter gene expression under a Fus1 promoter, which allows for a convenient screen using reporter gene activation. In certain embodiments, a GPCR is expressed in a sensor cell and is coupled to the yeast pheromone mating system such that GPCR binding activates the Fus1 promoter to express a downstream reporter gene, e.g., CrtI.

In certain embodiments, CrtB and/or CrtE are constitutively expressed, e.g., under the control of a constitutively active promoter such as pTef1.

In certain embodiments, a sensor cell of the present disclosure can be genetically engineered to express one or more enzymes involved in the synthesis of the reporter. For example, but not by way of limitation, a sensor cell can be genetically engineered to express one or more enzymes involved in the synthesis of lycopene. Non-limiting examples of such enzymes are disclosed in the Example 1, e.g., HMG1 and ERG20. In certain embodiments, a sensor cell of the present disclosure can be genetically engineered to comprise one or more copies of HMG1 and/or ERG20. In certain embodiments, a sensor cell of the present disclosure can be genetically engineered to comprise one or more copies of a catalytic domain of HMG1. In certain embodiments, a sensor cell of the present disclosure is genetically engineered to include a copy of CrtE (e.g., operably linked to a constitutively active promoter), a copy of CrtB (e.g., operably linked to a constitutively active promoter), two or three copies (e.g., three copies) of CrtI (e.g., operably linked to an inducible promoter), at least one copy of FAD (e.g., operably linked to a constitutively active promoter) and a copy of HMG1 (e.g., operably linked to a constitutively active promoter). In certain embodiments, a sensor cell of the present disclosure is genetically engineered to include a copy of CrtE (e.g., operably linked to a constitutively active promoter), a copy of CrtB (e.g., operably linked to a constitutively active promoter), two or three copies (e.g., three copies) of CrtI (e.g., operably linked to an inducible promoter), at least one copy of FAD (e.g., operably linked to a constitutively active promoter), a copy of HMG1 (e.g., operably linked to a constitutively active promoter) and a copy of ERG20 (e.g., operably linked to a constitutively active promoter).

In certain embodiments, a sensor cell of the present disclosure can be genetically engineered to reduce the expression of one or more enzymes involved in the synthesis of squalene. Non-limiting examples of such enzymes are disclosed in the Example 1, e.g., ERG9. For example, but not by way of limitation, the ERG9 gene can be disrupted in a sensor cell to reduce expression of ERG9 in the cell. For example, and not by way of limitation, a sensor cell disclosed herein, e.g., a sensor cell genetically engineered to include a copy of CrtE (e.g., operably linked to a constitutively active promoter), a copy of CrtB (e.g., operably linked to a constitutively active promoter), two or three copies (e.g., three copies) of CrtI (e.g., operably linked to an inducible promoter), at least one copy of FAD (e.g., operably linked to a constitutively active promoter), a copy of HMG1 (e.g., operably linked to a constitutively active promoter) and a copy of ERG20 (e.g., operably linked to a constitutively active promoter) can be further modified to reduce the expression of the ERG9 gene.

In certain embodiments, where the pathway includes the yeast pheromone sensing pathway, a nucleic acid encoding the reporter is operably linked to at least a transcription controlling portion of the Fus1 promoter, for example, but not limited to, an activating sequence located in the region (−300) to (+400) of the Fus1 gene (Gene ID: 850330). In certain non-limiting embodiments, where the pathway includes the yeast pheromone sensing pathway, a nucleic acid encoding the reporter is operably linked to a Ste12-binding element [(A/T)GAAACA], such that binding of Ste12 acts as a transactivator of the expression of the reporter. In certain embodiments, where the pathway includes the yeast pheromone sensing pathway, a nucleic acid encoding the reporter is alternatively linked to one or more inducible promoter other than pFus1, e.g., pFus2, pFig2, and/or pAga1.

In certain embodiments, receptor-activation is linked to an engineered pheromone-responsive transcription factor, which binds a synthetic transcription controlling element distinct from the Ste12-binding element. The transcription factor Ste12 is composed of a DNA-binding domain, a pheromone responsive domain and an activation domain. The feasibility of engineering Ste12 to bind to non-natural control elements but remain to activate transcription in a pheromone-responsive manner has been shown.

In certain embodiments, the sensor cell can include one or more nucleic acids encoding the receptor, reporter and/or the one or more enzymes involved in the synthesis of lycopene. In certain embodiments, the one or more nucleic acids can be inserted into the genome of the sensor cell. For example, but not by way of limitation, the one or more nucleic acids can be inserted into the Ste2, Ste3, ARS208a, Leu2 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, the reporter is the expression of a peptide including redox peptides and metal-chelating peptides. Non-limiting examples of redox and metal-chelating peptides are disclosed in Example 16. In certain embodiments, the expression of the redox peptide and/or metal-chelating peptide can be detected by voltammetry, square-wave voltammetry and/or chronopotentiometry.

The presently disclosed sensor cells further include a reporter gene, e.g., a nucleic acid encoding a reporter. In certain non-limiting embodiments, the sensor cell comprises a receptor that binds to a polypeptide variant and/or wild type polypeptide, e.g., protein variant and/or wild type protein, wherein the receptor is coupled to a reporter gene such that when the polypeptide variant and/or wild type polypeptide, e.g., protein variant and/or wild type protein, binds to the receptor, expression of the reporter gene is altered, e.g., increased or decreased. In certain embodiments, the receptor is coupled to a detectable reporter gene such that when a protein variant binds to the receptor, expression of the reporter gene is increased. In certain embodiments, the receptor is coupled to a detectable reporter gene such that when the protein variant and/or wild type protein binds to the receptor, expression of the reporter gene is inhibited (for example, by binding of a transcriptional repressor).

### VII. Sensor Cell Compositions

The presently disclosed subject matter provides compositions comprising one or more sensor cells. For example, but not by way of limitation, the present disclosure provides compositions that include at least two sensor cells that can communicate with one another to promoting intercellular signaling between the least two cells for detecting an analyte disclosed herein.

Additional non-limiting examples of compositions comprising two or more cells are disclosed in WO 2020/0251697, the contents of which are disclosed herein in its entirety.

In certain embodiments, a sensor cell composition comprising at least two sensor cells includes a first sensor cell that includes (a) a receptor that binds to an analyte in a sample and (b) expresses a secretable ligand. In certain embodiments, the first sensor cell further includes a reporter gene that is expressed upon activation of the receptor by the analyte. In certain embodiments, the composition includes a second cell that includes (a) a receptor that binds to and is activated by the secretable ligand expressed by the first sensor cell and, optionally, (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell. Alternatively, the reporter gene of the second cell is different from the reporter gene of the first sensor cell. In certain embodiments, the receptor genes are different in both cells, e.g., express reporter genes that encode different fluorescent protein and/or encode different visible light pigments.

In certain embodiments, the secretable ligand expressed by the first sensor cell selectively interacts with and activates the second receptor expressed by the second sensor cell. In certain embodiments, the secretable ligand is a peptide as described herein. In certain embodiments, the secretable ligand is the analyte being detected. In certain embodiments, the receptor expressed by the second cell is a GPCR. In certain embodiments, the GPCR is a Ste2 receptor as described herein. For example, but not by way of limitation, the GPCR is a Ste2 receptor from S. cerevisiae. In certain embodiments, the secretable ligand is a peptide that binds to and activates the Ste2 receptor from S. cerevisiae, e.g., a mating peptide that binds to and activates the Ste2 receptor from S. cerevisiae. In certain embodiments, the mating peptide can be the S. cerevisiae mating peptide disclosed in Table 11. Alternatively, the secretable ligand can be any one of the ligands disclosed in Table 11 and the second cell can express, e.g., genetically engineered to express, a heterologous receptor that specifically binds to the secretable ligand, e.g., a GPCR recited in Tables 5-7 or in the examples.

### A. Sensor Cell Compositions for Detecting Fungal-Derived Analytes

In certain embodiments, the present disclosure provides compositions that include at least two sensor cells that can communicate with one another to promoting intercellular signaling between the least two cells for detecting an analyte derived from a fungal species that causes a healthcare-associated infection.

In certain embodiments, the heterologous receptor binds to an analyte derived from a fungal species that causes aspergillosis, blastomycosis, coccidiodmycosis, pneumocystis and/or candidiasis.

In certain embodiments, the heterologous receptor binds to an analyte derived from a fungal species selected from A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, the present disclosure provides compositions that include at least two sensor cells that can communicate with one another to promote intercellular signaling between the least two cells for detecting an analyte derived from a species of Aspergillus, e.g., A. nidulans, A. fumigatus, A. terreus, A. flavus, A. niger, A. clavatus, A. oryzae, A. novofumigatus, A. lentulus, A. viridinutans, A. udagawae and/or N. fischeri, in a sample. In certain embodiments, the species is A. fumigatus. In certain embodiments, the use of such compositions can increase the sensitivity to Aspergillus by amplifying the expression of the reporter upon detection of an Aspergillus species.

In certain embodiments, the composition can include two or more, three or more, four or more or five or more sensor cells that have been genetically-engineered to communicate with each other to allow detection of an analyte derived from a fungal species disclosed herein, e.g., A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis. In certain embodiments, the sensor cells of the composition allow the rapid detection of an analyte derived from the fungal species in a sample. In certain embodiments, the sensor cells of the composition allow detection of the fungal species in a sample in less than about 2 hours, in less than about 1.5 hours, in less than about 1 hour or in less than about 30 minutes. In certain embodiments, the composition allows detection in less than about 1 hour. In certain embodiments, the composition allows detection in less than about 30 minutes.

In certain embodiments, the composition can include two or more, three or more, four or more or five or more sensor cells that have been genetically-engineered to communicate with each other to allow detection of an analyte derived from a species of Aspergillus, e.g., A. nidulans, A. fumigatus, A. terreus, A. flavus, A. niger, A. clavatus, A. oryzae, A. novofumigatus, A. lentulus, A. viridinutans, A. udagawae and/or N. fischeri, in a sample. In certain embodiments, the species is A. fumigatus. In certain embodiments, the sensor cells of the composition allow the rapid detection of an analyte derived from a species of Aspergillus. e.g., an analyte derived from A. fumigatus, in a sample. In certain embodiments, the sensor cells of the composition allow detection of a species of Aspergillus in a sample in less than about 2 hours, in less than about 1.5 hours, in less than about 1 hour or in less than about 30 minutes. In certain embodiments, the sensor cells of the composition allow detection of a species of Aspergillus in a sample detection in less than about 1 hour. In certain embodiments, the sensor cells of the composition allow detection of a species of Aspergillus in a sample detection in less than about 30 minutes.

In certain embodiments, a composition comprising at least two sensor cells includes at least a first sensor cell that includes (a) a receptor that binds to an analyte derived from a fungal species disclosed herein, e.g., A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis, and (b) expresses a secretable ligand. In certain embodiments, the first sensor cell further includes a reporter gene that is expressed upon activation of the receptor by the analyte derived from the fungal species.

In certain embodiments, a composition comprising at least two sensor cells includes a first sensor cell that includes (a) a receptor that binds to an analyte derived from a species of Aspergillus, e.g., A. nidulans, A. fumigatus, A. terreus, A. flavus, A. niger, A. clavatus, A. oryzae, A. novofumigatus, A. lentulus, A. viridinutans, A. udagawae and/or N. fischeri, in a sample and (b) expresses a secretable ligand. In certain embodiments, the species is A. fumigatus. In certain embodiments, the first sensor cell further includes a reporter gene that is expressed upon activation of the receptor by the analyte derived from the Aspergillus species.

In certain embodiments, the composition includes a second cell that includes (a) a receptor that binds to and is activated by the secretable ligand expressed by the first sensor cell and (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell.

In certain embodiments, a composition comprising at least two sensor cells includes a first sensor cell that includes (a) a receptor that binds to an analyte derived from A. fumigatus in a sample, e.g., AfuSte2, and (b) expresses a secretable ligand, e.g., ScPep. In certain embodiments, the composition includes a second cell that includes (a) a receptor, e.g., Ste2, that binds to and is activated by the secretable ligand, e.g., ScPep, expressed by the first sensor cell and (b) expresses the secretable ligand, e.g., ScPep. Alternatively, the second cell does not express the secretable ligand. In certain embodiments, the second cell can express the analyte detected by the first cell, e.g., AfuSte2. In certain embodiments, both cells further express a reporter gene that is expressed upon activation of the in each cell. In certain embodiments, the receptor genes are the same in both cells, e.g., a reporter gene that encodes a fluorescent protein or a visible light pigment. In certain embodiments, the receptor genes are different in both cells, e.g., express reporter genes that encode different fluorescent protein and/or encode different visible light pigments.

### B. Sensor Cell Compositions for Detecting Viruses

In certain embodiments, the present disclosure provides compositions that include at least two sensor cells that can communicate with one another to promoting intercellular signaling between the least two cells for detecting an analyte derived from a virus disclosed herein. For example, but not by way of limitation, the present disclosure provides compositions that include at least two sensor cells for detecting an analyte derived from a respiratory virus, a hemorrhagic virus, a gastrointestinal virus, an exanthematous virus, a hepatitis virus, a sexually transmitted virus and/or a neurological virus.

In certain embodiments, the present disclosure provides compositions that include at least two sensor cells that can communicate with one another to promoting intercellular signaling between the least two cells for detecting an analyte derived from a respiratory virus in a sample. In certain embodiments, the respiratory virus is a coronavirus, e.g., SARS-CoV-2. In certain embodiments, the use of such compositions can increase the sensitivity to a respiratory virus by amplifying the expression of the reporter upon detection of the respiratory virus.

In certain embodiments, the present disclosure provides compositions that include at least two sensor cells that can communicate with one another to promoting intercellular signaling between the least two cells for detecting an analyte derived from a hemorrhagic virus in a sample. In certain embodiments, the hemorrhagic virus is an ebolavirus. In certain embodiments, the use of such compositions can increase the sensitivity to an ebolavirus by amplifying the expression of the reporter upon detection of the ebolavirus.

In certain embodiments, the composition can include two or more, three or more, four or more or five or more sensor cells that have been genetically-engineered to communicate with each other to allow detection of an analyte derived from a virus disclosed herein, e.g., a respiratory virus, a hemorrhagic virus, a gastrointestinal virus, an exanthematous virus, a hepatitis virus and/or a neurological virus. In certain embodiments, the sensor cells of the composition allow the rapid detection of an analyte derived from the virus in a sample. In certain embodiments, the sensor cells of the composition allow detection of the virus in a sample in less than about 2 hours, in less than about 1.5 hours, in less than about 1 hour or in less than about 30 minutes. In certain embodiments, the sensor cells of the composition allow detection of the virus in a sample in less than about 1 hour. In certain embodiments, the sensor cells of the composition allow detection of the virus in a sample in less than about 30 minutes.

In certain embodiments, the composition can include two or more, three or more, four or more or five or more sensor cells that have been genetically-engineered to communicate with each other to allow detection of an analyte derived from a coronavirus in a sample. In certain embodiments, the coronavirus is SARS-CoV-2. In certain embodiments, the sensor cells of the composition allow the rapid detection of an analyte derived from SARS-CoV-2 in a sample. In certain embodiments, the sensor cells of the composition allow detection of SARS-CoV-2 in a sample in less than about 2 hours, in less than about 1.5 hours, in less than about 1 hour or in less than about 30 minutes. In certain embodiments, the sensor cells of the composition allow detection of SARS-CoV-2 in less than about 1 hour. In certain embodiments, the sensor cells of the composition allow detection of SARS-CoV-2 in less than about 30 minutes.

In certain embodiments, the composition can include two or more, three or more, four or more or five or more sensor cells that have been genetically-engineered to communicate with each other to allow detection of an analyte derived from a hemorrhagic virus in a sample. In certain embodiments, the hemorrhagic virus is an ebolavirus. In certain embodiments, the sensor cells of the composition allow the rapid detection of an analyte derived from an ebolavirus in a sample. In certain embodiments, the sensor cells of the composition allow detection of ebolavirus in a sample in less than about 2 hours, in less than about 1.5 hours, in less than about 1 hour or in less than about 30 minutes. In certain embodiments, the sensor cells of the composition allow detection of ebolavirus in less than about 1 hour. In certain embodiments, the sensor cells of the composition allow detection of ebolavirus in less than about 30 minutes.

In certain embodiments, a composition comprising at least two sensor cells includes a first sensor cell that includes (a) a receptor that binds to an analyte derived from a virus disclosed herein, e.g., a respiratory virus, a hemorrhagic virus, a gastrointestinal virus, an exanthematous virus, a sexually transmitted virus, a hepatitis virus and/or a neurological virus, and (b) expresses a secretable ligand. In certain embodiments, the first sensor cell further includes a reporter gene that is expressed upon activation of the receptor by the analyte derived from the virus. In certain embodiments, the composition includes a second cell that includes (a) a receptor that binds to and is activated by the secretable ligand expressed by the first sensor cell and, optionally, (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell. Alternatively, the reporter gene of the second cell is the different from the reporter gene of the first sensor cell. In certain embodiments, the receptor genes are different in both cells, e.g., express reporter genes that encode different fluorescent protein and/or encode different visible light pigments.

In certain embodiments, a composition comprising at least two sensor cells includes a first sensor cell that includes (a) a receptor that binds to an analyte derived from a coronavirus, e.g., SARS-CoV-2, and (b) expresses a secretable ligand. In certain embodiments, the first sensor cell further includes a reporter gene that is expressed upon activation of the receptor by the analyte derived from the virus. In certain embodiments, the composition includes a second cell that includes (a) a receptor that binds to and is activated by the secretable ligand expressed by the first sensor cell and, optionally, (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell. Alternatively, the reporter gene of the second cell is the different from the reporter gene of the first sensor cell. In certain embodiments, the secretable ligand can be the mating peptide of the receptor expressed by the second cell, e.g., the mating peptide can be ScPep and the receptor of the second cell can be ScSte2. In certain embodiments, the first or second cell can further express a protease, e.g., Bar1.

In certain embodiments, a composition comprising at least two sensor cells includes a first sensor cell that includes (a) a receptor that binds to an analyte derived from an ebolavirus, e.g., Zaire ebolavirus, and (b) expresses a secretable ligand. In certain embodiments, the first sensor cell further includes a reporter gene that is expressed upon activation of the receptor by the analyte derived from the ebolavirus. In certain embodiments, the composition includes a second cell that includes (a) a receptor that binds to and is activated by the secretable ligand expressed by the first sensor cell and, optionally, (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell. Alternatively, the reporter gene of the second cell is the different from the reporter gene of the first sensor cell. In certain embodiments, the secretable ligand can be the mating peptide of the receptor expressed by the second cell, e.g., the mating peptide can be ScPep and the receptor of the second cell can be ScSte2. In certain embodiments, the first or second cell can further express a protease, e.g., Bar1.

In certain embodiments, a composition of the present disclosure can include two or more cells that detect two or more analytes from a virus. For example, but not by way of limitation, a composition of the present disclosure can include two or more cells, where each cell comprises a heterologous receptor specific to a different analyte derived from the virus. In certain embodiments, a composition can include three or more, four or more, five or more, six or more, seven or more or eight or more cells, where each cell comprises a heterologous receptor specific to a different analyte derived from the virus.

In certain embodiments, a composition can include a first cell that expresses a heterologous receptor that binds to a first analyte derived from a coronavirus, e.g., SARS-CoV-2. In certain embodiments, the first analyte can be derived from the nucleocapsid protein of the coronavirus, e.g., SARS-CoV-2. The composition can include a second cell that expresses a heterologous receptor that binds to a second analyte derived from the coronavirus, e.g., SARS-CoV-2. In certain embodiments, the second analyte can be derived from the S protein of the coronavirus, e.g., SARS-CoV-2 (e.g., an analyte comprising a sequence set forth in Table 3). In certain embodiments, the composition can further include a third cell that expresses a heterologous receptor that binds to a third analyte derived from the coronavirus, e.g., SARS-CoV-2. In certain embodiments, the third analyte can be derived from the S protein or the nucleocapsid protein of the coronavirus, e.g., SARS-CoV-2 (e.g., an analyte comprising a sequence set forth in Table 3).

In certain embodiments, a composition can include a first cell that expresses a heterologous receptor that binds to a first analyte derived from an ebolavirus, e.g., the Zaire Ebolavirus. In certain embodiments, the first analyte can be derived from the small secreted glycoprotein of the ebolavirus, e.g., the Zaire ebolavirus (e.g., an analyte comprising a sequence set forth in Table 3). The composition can include a second cell that expresses a heterologous receptor that binds to a second analyte derived from the ebolavirus, e.g., the Zaire ebolavirus. In certain embodiments, the second analyte can be derived from the small secreted glycoprotein of the ebolavirus, e.g., the Zaire ebolavirus (e.g., an analyte comprising a sequence set forth in Table 3). In certain embodiments, the composition can further include a third cell that expresses a heterologous receptor that binds to a third analyte derived from the ebolavirus, e.g., the Zaire ebolavirus. In certain embodiments, the third analyte can be derived from the small secreted glycoprotein of the ebolavirus, e.g., the Zaire ebolavirus (e.g., an analyte comprising a sequence set forth in Table 3).

### C. Sensor Cell Compostions for Detecting Protein Variants

The presently disclosed subject matter provides compositions comprising one or more sensor cells. For example, but not by way of limitation, the present disclosure provides compositions that include at least two sensor cells that can communicate with one another to promoting intercellular signaling between the least two cells for detecting a protein variant or a wild type protein.

In certain embodiments, the present disclosure provides compositions that include at least two sensor cells that can communicate with one another to promoting intercellular signaling between the least two cells for detecting the presence of one or more protein variants in a sample.

In certain embodiments, the present disclosure provides compositions that include at least two sensor cells that can communicate with one another to promoting intercellular signaling between the least two cells for detecting the presence of one or more protein variants in a sample. In certain embodiments, the use of such compositions can increase the sensitivity to the wild type polypeptide and polypeptide variant, e.g., protein variant, by amplifying the expression of the reporter upon detection of the protein variant and/or wild type polypeptide.

In certain embodiments, the composition can include two or more, three or more, four or more or five or more sensor cells that have been genetically-engineered to communicate with each other to allow detection of one or more protein variants in a sample. In certain embodiments, the sensor cells of the composition allow the rapid detection of a wild type protein and/or a variant of the protein in a sample. In certain embodiments, the sensor cells of the composition allow detection of the wild type protein and/or a variant of the protein in a sample in less than about 2 hours, in less than about 1.5 hours, in less than about 1 hour or in less than about 30 minutes. In certain embodiments, the sensor cells of the composition allow detection of the wild type protein and/or a variant of the protein in a sample in less than about 1 hour. In certain embodiments, the sensor cells of the composition allow detection of the wild type protein and/or a variant of the protein in a sample in less than about 30 minutes.

In certain embodiments, the composition can include two or more, three or more, four or more or five or more sensor cells that have been genetically-engineered to communicate with each other to allow detection of a wild type protein and/or a variant of the protein derived from a coronavirus, e.g., SARS-CoV-2, in a sample. In certain embodiments, the sensor cells of the composition allow the rapid detection of a wild type protein and/or a variant of the protein from a coronavirus, e.g., SARS-CoV-2, in a sample. In certain embodiments, the sensor cells of the composition allow detection of a wild type protein and/or a variant of the protein e.g., an S protein, from SARS-CoV-2 in a sample in less than about 2 hours, in less than about 1.5 hours, in less than about 1 hour or in less than about 30 minutes. In certain embodiments, the sensor cells of the composition allow detection of a wild type protein and/or a variant of the protein e.g., an S protein, from SARS-CoV-2 in a sample in less than about 1 hour. In certain embodiments, the sensor cells of the composition allow detection of a wild type protein and/or a variant of the protein e.g., an S protein, from SARS-CoV-2 in a sample in less than about 30 minutes.

In certain embodiments, a composition comprising at least two sensor cells includes a first sensor cell that includes (a) a receptor that binds to a variant of a protein of a coronavirus, e.g., SARS-CoV-2, and the wild type protein, (b) a protease for cleaving either the variant of the protein or the wild type protein and (c) expresses a secretable ligand. In certain embodiments, the first sensor cell further includes a reporter gene that is expressed upon activation of the receptor by the wild type protein or variant derived from a coronavirus, e.g., SARS-CoV-2, that is not cleaved by the protease. In certain embodiments, the protease cleaves and digests either the variant of the protein or the wild type protein, so it is unable to bind and activate the receptor. In certain embodiments, the protease cleaves the protein variant. In certain embodiments, the protease cleaves the wild type protein.

In certain embodiments, the composition includes a second cell that includes (a) a receptor that binds to and is activated by the secretable ligand expressed by the first sensor cell and, optionally, (b) expresses the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell. Alternatively, the reporter gene of the second cell is different from the reporter gene of the first sensor cell and/or the receptor of the first cell is the same as the receptor of the second cell. In certain embodiments, the receptor genes are different in both cells, e.g., express reporter genes that encode different fluorescent protein and/or encode different visible light pigments.

In certain embodiments, a composition comprising at least two sensor cells includes a first sensor cell that includes (a) a receptor that binds to a variant of a protein and the wild type protein and (b) a protease for cleaving either the variant of the protein or the wild type protein. In certain embodiments, the first sensor cell further includes a reporter gene that is expressed upon activation of the receptor by the protein variant or wild type protein that is not cleaved by the protease. In certain embodiments, the protease cleaves and digests the variant of the protein or the wild type protein, so it is unable to bind and activate the receptor. For example, but not by way of limitation, the first sensor cell includes (a) a receptor that binds to a variant of a protein and the wild type protein and (b) a protease for cleaving the wild type protein, e.g., so that is it unable to activate the receptor. Alternatively, the first sensor cell includes (a) a receptor that binds to a variant of a protein and the wild type protein and (b) a protease for cleaving the variant of the protein, e.g., so that is it unable to activate the receptor. In certain embodiments, the first cell further expresses a secretable ligand upon the activation of the receptor.

In certain embodiments, the composition includes a second cell that includes a receptor that binds to and is activated by the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell and/or the receptor of the first cell is the same as the receptor of the second cell. In certain embodiments, the second cell does not express a protease for cleaving and digesting the protein variant and/or wild type protein.

In certain embodiments, a composition comprising at least two sensor cells includes a first sensor cell that includes (a) a receptor that binds to the wild type and one or more variants of a protein of a coronavirus, e.g., SARS-CoV-2, and (b) a protease for cleaving the variant of the protein of a coronavirus, e.g., SARS-CoV-2. In certain embodiments, the first sensor cell further includes a reporter gene that is expressed upon activation of the receptor by the non-cleaved wild type protein from a coronavirus, e.g., SARS-CoV-2. In certain embodiments, the first cell further expresses a secretable ligand. In certain embodiments, the protease cleaves and digests the variant of the protein of a coronavirus, e.g., SARS-CoV-2, so it is unable to bind and activate the receptor of the first cell. In certain embodiments, the composition includes a second cell that includes a receptor that binds to and is activated by the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell and/or the receptor of the first cell is the same as the receptor of the second cell. Alternatively, the reporter gene of the second cell is different from the reporter gene of the first sensor cell and/or the receptor of the first cell is the same as the receptor of the second cell. In certain embodiments, the receptor genes are different in both cells, e.g., express reporter genes that encode different fluorescent protein and/or encode different visible light pigments. In certain embodiments, the second cell does not express a protease for cleaving and digesting the protein variant from a coronavirus, e.g., SARS-CoV-2. In certain embodiments, if the protein variant is present in the sample and the wild type protein is not in the sample, then the receptor of the first sensor is not activated so that the reporter and the secretable ligand are not expressed and the receptor of the second sensor cell is not activated. Alternatively, if the protein variant is not present in the sample and the wild type protein is present in the sample, then the receptor of the first sensor is activated so that the reporter and the secretable ligand are expressed and the receptor of the second sensor cell is activated.

In certain embodiments, a composition comprising at least two sensor cells includes a first sensor cell that includes (a) a receptor that binds to the wild type and one or more variants of a protein of a coronavirus, e.g., SARS-CoV-2, and (b) a protease for cleaving the wild type protein of a coronavirus, e.g., SARS-CoV-2. In certain embodiments, the first sensor cell further includes a reporter gene that is expressed upon activation of the receptor by the non-cleaved protein variant from a coronavirus, e.g., SARS-CoV-2. In certain embodiments, the first cell further expresses a secretable ligand. In certain embodiments, the protease cleaves and digests the wild type protein of a coronavirus, e.g., SARS-CoV-2, so it is unable to bind and activate the receptor of the first cell. In certain embodiments, the composition includes a second cell that includes a receptor that binds to and is activated by the secretable ligand. In certain embodiments, the second sensor cell can also include a reporter gene that is expressed upon the binding of the secretable ligand to the receptor it expresses. In certain embodiments, the reporter gene of the second cell is the same as the reporter gene of the first sensor cell and/or the receptor of the first cell is the same as the receptor of the second cell. Alternatively, the reporter gene of the second cell is different from the reporter gene of the first sensor cell and/or the receptor of the first cell is the same as the receptor of the second cell. In certain embodiments, the second cell does not express a protease for cleaving and digesting the wild type protein from a coronavirus, e.g., SARS-CoV-2. In certain embodiments, if the wild type protein is present in the sample and the variant protein is not in the sample, then the receptor of the first sensor is not activated so that the reporter and the secretable ligand are not expressed and the receptor of the second sensor cell is not activated. Alternatively, if the wild type is not present in the sample and the protein variant is present in the sample, then the receptor of the first sensor is activated so that the reporter and the secretable ligand are expressed and the receptor of the second sensor cell is activated.

### VIII. Methods of Use

The disclosed subject matter provides a method for detecting the presence of an analyte in a sample using one or more sensor cells disclosed herein.

In certain embodiments, the method can include culturing the sensor cell for an effective period of time. In certain embodiments, the effective period of time can be hours (e.g., about 24 hours, about 18 hours, about 12 hours, about 8 hours, about 6 hours, about 4 hours, about 3 hours, or about 2 hours) or minutes (e.g., about 90 minutes, about 60 minutes, about 45 minutes, about 30 minutes, about 20 minutes, about 15 minutes, about 10 minutes, about 5 minutes, about 3 minutes, about 2 minutes, or about 1 minute). In certain embodiments, the effective period of time can be less than about 30 minutes, less than about 20 minutes, less than about 10 minutes or less than about 5 minutes. In certain embodiments, the method can include culturing the sensor cell in greater than about 6% yeast extract peptone dextrose (YPD), e.g., about 10% YPD. In certain embodiments, the method can include culturing the sensor cell in about 30% YPD. In certain embodiments, the method can include culturing the sensor cell in about 5% YPD.

In certain embodiments, the method can include determining the expression of the reporter gene, e.g., the reporter. In certain embodiments, determining whether a reporter gene is expressed comprises detecting the expression of the reporter gene by the naked eye and does not require instrumentation. In certain non-limiting embodiments, the reporter is lycopene. In certain embodiments, the reporter is a fluorescent protein.

In certain embodiments, the reporter can be detected within about 2 hours after contacting a sensor cell with the sample, e.g., within about 1.5 hours, within about 1 hours, within about 45 minutes, within about 30 minutes or within about 15 minutes after contacting a sensor cell with the sample.

The method for determining whether the reporter gene is or has been expressed depends upon the particular reporting gene used. If the reporter gene produces a visibly detectable product, such as lycopene, it can be detected with the naked eye or colorimetrically. Means of detection of reporters known in the art can be used.

### A. Methods of Detecting Fungal Species

In certain embodiments, the present disclosure provides a method for detecting the presence of an analyte derived from one or more fungal species disclosed herein. For example, but not by way of limitation, the present disclosure provides a method for detecting the presence of an analyte, e.g., a peptide analyte, derived from one or more fungal species that causes a healthcare-associated infection.

In certain embodiments, the present disclosure provides a method for detecting the presence of an analyte, e.g., a peptide analyte, derived from one or more fungal species that causes aspergillosis, blastomycosis, coccidiomycosis, pneumocystis or candidiasis. For example, but not by way of limitation, the present disclosure provides a method for detecting the presence aspergillosis, blastomycosis, coccidiomycosis, pneumocystis and/or candidiasis in a patient, e.g., by detecting the presence of an analyte, e.g., a peptide analyte, derived from a fungal species that causes aspergillosis, blastomycosis, coccidiomycosis, pneumocystis and/or candidiasis.

In certain embodiments, the methods of the present disclosure can be used to diagnosis a subject with aspergillosis, blastomycosis, coccidiomycosis, pneumocystis and/or candidiasis, e.g., so the subject can be treated.

In certain embodiments, the present disclosure provides a method for detecting the presence of aspergillosis in a patient, e.g., by detecting the presence of an analyte, e.g., a peptide analyte, derived from a species of the Aspergillus genus, e.g., A. fumigatus.

In certain embodiments, the present disclosure provides methods for detecting aspergillosis in a subject, e.g., by detecting the presence of one or more species of the genus Aspergillus, e.g., A. nidulans, A. fumigatus, A. terreus, A. flavus, A. niger, A. clavatus, A. oryzae, A. novofumigatus, A. lentulus, A. viridinutans, A. udagawae and/or N. fischeri, in a sample obtained from the subject. Non-limiting examples of clinically relevant samples are disclosed herein.

In certain embodiments, the present disclosure provides a method for detecting the presence of a fungal infection in a patient that is caused by a fungal species disclosed herein, e.g., by detecting the presence of an analyte, e.g., a peptide analyte, derived from the fungal species.

In certain embodiments, the present disclosure provides a method for detecting the presence of an analyte, e.g., a peptide analyte, derived from one or more fungal species selected from A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, the present disclosure provides a method for detecting the presence of an analyte, e.g., a peptide analyte, derived from a species of the Aspergillus genus, e.g., A. nidulans, A. fumigatus, A. terreus, A. flavus, A. niger, A. clavatus, Neosartorya fischeri, A. oryzae, A. novofumigatus, A. lentulus, A. viridinutans and/or A. udagawae. In certain embodiments, the present disclosure provides a method for detecting the presence of an analyte, e.g., a peptide analyte, derived from A. fumigatus.

In certain embodiments, a method of the present disclosure includes contacting a sensor cell or a sensor cell composition of the present disclosure.

In certain embodiments, the method includes contacting the sample with a sensor cell (e.g., a yeast sensor cell) expressing a receptor (e.g., a heterologous GPCR receptor) that binds to an analyte indicative of the presence of one or more fungal species, e.g., one or more species of the Aspergillus genus. In certain embodiments, the binding of the analyte to the receptor results in the expression of a reporter to indicate the presence of the agent (e.g., increased expression of a reporter).

The present disclosure provides methods for detecting the presence of one or more fungal species disclosed herein. For example, but not by way of limitation, the present disclosure provides a method for detecting the presence of one or more fungal species disclosed herein. In certain embodiments, the method can include detecting the presence of a analyte derived from a fungal species includes (a) contacting a sample with a sensor cell expressing a heterologous GPCR polypeptide that binds to the analyte coupled to a reporter gene such that when the analyte binds to the receptor, expression of the reporter gene is induced and lycopene is produced; (b) culturing the sensor yeast cell for an effective period of time; and (c) determining whether the reporter has been produced. In certain embodiments, determining whether the reporter is produced is performed by the naked eye and does not require instrumentation. In certain non-limiting embodiments, the reporter is lycopene.

In certain embodiments, the present disclosure provides a method for detecting the presence of a species of the Aspergillus genus, e.g., A. fumigatus. In certain embodiments, the method can include detecting the presence of a analyte derived from a species of the Aspergillus genus, e.g., A. fumigatus, by a method that includes (a) contacting a sample with a sensor cell expressing a heterologous GPCR polypeptide that binds to the analyte coupled to a reporter gene such that when the analyte binds to the receptor, expression of the reporter gene is induced and lycopene is produced; (b) culturing the sensor yeast cell for an effective period of time; and (c) determining whether the reporter has been produced. In certain embodiments, determining whether the reporter is produced is performed by the naked eye and does not require instrumentation. In certain non-limiting embodiments, the reporter is lycopene.

In certain embodiments, a method of detecting the presence of a fungal species, e.g., a species of the Aspergillus genus, e.g., A. fumigatus, in a sample, includes (a) contacting the sample with a sensor cell, e.g., sensor fungal cell, comprising a heterologous GPCR that binds to an analyte, e.g., peptide analyte, derived from the fungal species, e.g., the species of the Aspergillus genus, e.g., A. fumigatus, wherein the analyte is a ligand for the heterologous GPCR, (b) binding of the analyte present in the sample to the heterologous GPCR, wherein binding of the analyte, e.g., peptide analyte, to the heterologous GPCR triggers an appearance of a reporter, wherein the reporter is a visible light pigment, e.g., a biosynthesized visible light pigment and (c) detecting the appearance of the reporter by the naked eye, wherein the appearance of the reporter indicates the presence of the species of the fungal species, e.g., the species of the Aspergillus genus, e.g., A. fumigatus, in the sample. In certain embodiments, the biosynthesized visible light pigment is lycopene.

In certain embodiments, the present disclosure provides methods for detecting a fungal species in a clinically relevant sample. In certain embodiments, the samples that can be analyzed by the disclosed methods include body fluid samples such as, but not limited to, intestinal fluids, diarrhea or other feces, mucus (e.g., sputum), blood, cerebrospinal fluid, synovial fluid, lymph, pus, saliva, vomit, urine, bile, vaginal fluid, bronchoalveolar lavage and sweat. In certain embodiments, the sample is an environmental sample such as, but not limited to, water, air and soil samples. In certain embodiments, the sample is a culture supernatant.

### B. Methods of Detecting Viruses

The disclosed subject matter provides a method for detecting the presence of an analyte in a sample using one or more sensor cells disclosed herein. In certain embodiments, the present disclosure provides a method for detecting the presence of an analyte derived from one or more viruses disclosed herein in a sample, e.g., a sample from a subject. For example, but not by way of limitation, the present disclosure provides a method for detecting the presence of an analyte, e.g., a peptide analyte, derived from a respiratory virus, a hemorrhagic virus, a gastrointestinal virus, an exanthematous virus, a hepatitis virus, a sexually transmitted virus and/or a neurological virus.

In certain embodiments, the present disclosure provides a method for detecting the presence of an analyte, e.g., a peptide analyte, derived from a respiratory virus. In certain embodiments, the present disclosure provides a method for detecting the presence of an analyte, e.g., a peptide analyte, derived from a respiratory virus in a sample from a subject.

In certain embodiments, the method can be a diagnostic method, where the determination of the presence of a virus provides a diagnosis for the subject from which the sample was obtained, e.g., so the subject can be treated.

In certain embodiments, the present disclosure provides a method for detecting the presence of an analyte, e.g., a peptide analyte, derived from a coronavirus. In certain embodiments, the present disclosure provides a method for detecting the presence of an analyte, e.g., a peptide analyte, derived from SARS-CoV-2. In certain embodiments, the present disclosure provides a method for detecting the presence of an analyte, e.g., a peptide analyte, derived from a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, a SARS-CoV-2 omicron variant or a SARS-CoV-2 zeta variant. In certain embodiments, the present disclosure provides a method for detecting the presence of an analyte, e.g., a peptide analyte, derived from a variant of SARS-CoV-2, e.g., the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants. In certain embodiments, the present disclosure provides a method for detecting the presence of an analyte, e.g., a peptide analyte, of a coronavirus disclosed in Table 3.

In certain embodiments, the present disclosure provides a method for detecting the presence of an analyte, e.g., a peptide analyte, derived from a hemorrhagic virus. In certain embodiments, the present disclosure provides a method for detecting the presence of an analyte, e.g., a peptide analyte, derived from an ebolavirus. In certain embodiments, the present disclosure provides a method for detecting the presence of an analyte, e.g., a peptide analyte, derived from an ebolavirus species, e.g., the Zaire ebolavirus, Bundibugyo ebolavirus, Sudan ebolavirus, Bombali ebolavirus, Reston ebolavirus and/or Tai Forest ebolavirus. In certain embodiments, the present disclosure provides a method for detecting the presence of an analyte, e.g., a peptide analyte, derived from the Zaire ebolavirus. In certain embodiments, the present disclosure provides a method for detecting the presence of an analyte, e.g., a peptide analyte, of an ebolavirus disclosed in Table 3.

In certain embodiments, the present disclosure provides a method for detecting the presence of a viral infection in a patient that is caused by a virus disclosed herein by detecting the presence of an analyte, e.g., a peptide analyte, derived from the virus. In certain embodiments, the present disclosure provides a method for detecting the presence of a viral infection in a patient that is caused by a virus disclosed herein by contacting a sensor cell or a sensor cell composition of the present disclosure with a sample from the patient.

In certain embodiments, the present disclosure provides a method for detecting the presence of a neurological infection in a patient that is caused by a neurological virus disclosed herein by detecting the presence of an analyte, e.g., a peptide analyte, derived from the neurological virus.

In certain embodiments, the present disclosure provides a method for detecting the presence of a sexually transmitted disease in a patient that is caused by a sexually transmitted virus disclosed herein by detecting the presence of an analyte, e.g., a peptide analyte, derived from the sexually transmitted virus.

In certain embodiments, the present disclosure provides a method for detecting the presence of a gastrointestinal infection in a patient that is caused by a gastrointestinal virus disclosed herein by detecting the presence of an analyte, e.g., a peptide analyte, derived from the gastrointestinal virus.

In certain embodiments, the present disclosure provides a method for detecting the presence of hepatitis in a patient that is caused by a hepatitis virus disclosed herein by detecting the presence of an analyte, e.g., a peptide analyte, derived from the hepatitis virus.

In certain embodiments, the present disclosure provides a method for detecting the presence of a respiratory infection in a patient that is caused by a respiratory virus disclosed herein by detecting the presence of an analyte, e.g., a peptide analyte, derived from the respiratory virus.

In certain embodiments, the present disclosure provides a method for detecting the presence of a COVID-19 infection in a patient that is caused by a SARS-CoV-2 virus disclosed herein by detecting the presence of an analyte, e.g., a peptide analyte, derived from the SARS-CoV-2 virus. In certain embodiments, the present disclosure provides a method for detecting the presence of COVID-19 in a patient by detecting the presence of an analyte, e.g., a peptide analyte, derived from a variant of SARS-CoV-2, e.g., the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants. In certain embodiments, the present disclosure provides a method for detecting the presence of COVID-19 in a patient by detecting the presence of an analyte, e.g., a peptide analyte, disclosed in Table 3.

In certain embodiments, the present disclosure provides a method for detecting the presence of a hemorrhagic fever in a patient that is caused by a hemorrhagic virus, e.g., an ebolavirus disclosed herein, by detecting the presence of an analyte, e.g., a peptide analyte, derived from the hemorrhagic virus, e.g., an ebolavirus. In certain embodiments, the present disclosure provides a method for detecting the presence of Ebola hemorrhagic fever in a patient that is caused by an ebolavirus disclosed herein, by detecting the presence of an analyte, e.g., a peptide analyte, derived from an ebolavirus. In certain embodiments, the present disclosure provides a method for detecting the presence of Ebola hemorrhagic fever in a patient that is caused by an ebolavirus species disclosed herein, by detecting the presence of an analyte, e.g., a peptide analyte, derived from the ebolavirus species, e.g., the Zaire ebolavirus, Bundibugyo ebolavirus, Sudan ebolavirus, Bombali ebolavirus, Reston ebolavirus and/or Tai Forest ebolavirus. In certain embodiments, the present disclosure provides a method for detecting the presence of a hemorrhagic fever in a patient that is caused by a hemorrhagic virus, e.g., an ebolavirus disclosed herein, by detecting the presence of an analyte, e.g., a peptide analyte, disclosed in Table 3.

In certain embodiments, the method includes contacting the sample with a sensor cell (e.g., a yeast sensor cell) expressing a receptor (e.g., a heterologous GPCR receptor) that binds to an analyte indicative of the presence of one or more viruses disclosed herein. Examples of GPCRs for use in the disclosed methods are provided in Section IV and in the examples. In certain embodiments, the binding of the analyte to the receptor results in the expression of a reporter to indicate the presence of the agent (e.g., increased expression of a reporter).

In certain embodiments, the method includes contacting the sample with a sensor cell (e.g., a yeast sensor cell) expressing a receptor (e.g., a heterologous GPCR receptor) that binds to an analyte indicative of the presence of one or more respiratory viruses, e.g., one or more coronaviruses. Examples of GPCRs for use in the detecting a respiratory virus, e.g., a coronavirus, are provided in Section IV and the examples. In certain embodiments, the binding of the analyte to the receptor results in the expression of a reporter to indicate the presence of the agent (e.g., increased expression of a reporter).

In certain embodiments, the method includes contacting the sample with a sensor cell (e.g., a yeast sensor cell) expressing a receptor (e.g., a heterologous GPCR receptor) that binds to an analyte indicative of the presence of one or more hemorrhagic viruses, e.g., one or more ebolaviruses. Examples of GPCRs for use in the detecting a hemorrhagic virus, e.g., an ebolavirus, are provided in Section IV and the examples. In certain embodiments, the binding of the analyte to the receptor results in the expression of a reporter to indicate the presence of the agent (e.g., increased expression of a reporter).

The present disclosure provides methods for detecting the presence of one or more viruses disclosed herein. For example, but not by way of limitation, the present disclosure provides a method for detecting the presence of one or more viruses disclosed herein, e.g., a respiratory virus, a hemorrhagic virus, a gastrointestinal virus, an exanthematous virus, a hepatitis virus and/or a neurological virus. In certain embodiments, the method can include detecting the presence of an analyte derived from a virus includes (a) contacting a sample with a sensor cell expressing a heterologous GPCR polypeptide that binds to the analyte coupled to a reporter gene such that when the analyte binds to the receptor, expression of the reporter gene is induced and lycopene is produced; (b) culturing the sensor yeast cell for an effective period of time; and (c) determining whether the reporter has been produced. In certain embodiments, determining whether the reporter is produced is performed by the naked eye and does not require instrumentation. In certain non-limiting embodiments, the reporter is a visible light pigment, e.g., lycopene. Detection of the reporter indicates that the virus is present in the sample.

In certain embodiments, the present disclosure provides a method for detecting the presence of a respiratory virus. In certain embodiments, the method can include detecting the presence of an analyte derived from a coronavirus, e.g., SARS-CoV-2, by a method that includes (a) contacting a sample with a sensor cell expressing a heterologous GPCR polypeptide that binds to the analyte coupled to a reporter gene such that when the analyte binds to the receptor, expression of the reporter gene is induced and lycopene is produced; (b) culturing the sensor yeast cell for an effective period of time; and (c) determining whether the reporter has been produced. In certain embodiments, determining whether the reporter is produced is performed by the naked eye and does not require instrumentation. In certain non-limiting embodiments, the reporter is a visible light pigment, e.g., lycopene. Detection of the reporter indicates that the coronavirus, e.g., SARS-CoV-2, is present in the sample.

In certain embodiments, the present disclosure provides a method for detecting the presence of a hemorrhagic virus. In certain embodiments, the method can include detecting the presence of an analyte derived from the ebolavirus by a method that includes (a) contacting a sample with a sensor cell expressing a heterologous GPCR polypeptide that binds to the analyte coupled to a reporter gene such that when the analyte binds to the receptor, expression of the reporter gene is induced and lycopene is produced; (b) culturing the sensor yeast cell for an effective period of time; and (c) determining whether the reporter has been produced. In certain embodiments, determining whether the reporter is produced is performed by the naked eye and does not require instrumentation. In certain non-limiting embodiments, the reporter is a visible light pigment, e.g., lycopene. Detection of the reporter indicates that the ebolavirus is present in the sample.

In certain embodiments, a method of detecting the presence of a virus disclosed herein, e.g., a respiratory virus, a hemorrhagic virus, a gastrointestinal virus, an exanthematous virus, a hepatitis virus and/or a neurological virus, in a sample, includes (a) contacting the sample with a sensor cell, e.g., sensor fungal cell, comprising a heterologous GPCR that binds to an analyte, e.g., peptide analyte, derived from the virus, wherein the analyte is a ligand for the heterologous GPCR, (b) binding of the analyte present in the sample to the heterologous GPCR, wherein binding of the analyte, e.g., peptide analyte, to the heterologous GPCR triggers an appearance of a reporter, wherein the reporter is a visible light pigment, e.g., a biosynthesized visible light pigment and (c) detecting the appearance of the reporter by the naked eye, wherein the appearance of the reporter indicates the presence of the virus, e.g., a respiratory virus, a hemorrhagic virus, a gastrointestinal virus, an exanthematous virus, a hepatitis virus and/or a neurological virus, in the sample. In certain embodiments, the biosynthesized visible light pigment is lycopene.

In certain embodiments, a method of detecting the presence of a respiratory virus disclosed herein, e.g., a coronavirus (e.g., SARS-CoV-2), in a sample, includes (a) contacting the sample with a sensor cell, e.g., sensor fungal cell, comprising a heterologous GPCR that binds to an analyte, e.g., peptide analyte, derived from the coronavirus (e.g., SARS-CoV-2), wherein the analyte is a ligand for the heterologous GPCR, (b) binding of the analyte present in the sample to the heterologous GPCR, wherein binding of the analyte, e.g., peptide analyte, to the heterologous GPCR triggers an appearance of a reporter, wherein the reporter is a visible light pigment, e.g., a biosynthesized visible light pigment and (c) detecting the appearance of the reporter by the naked eye, wherein the appearance of the reporter indicates the presence of the coronavirus (e.g., SARS-CoV-2) in the sample. In certain embodiments, the biosynthesized visible light pigment is lycopene.

In certain embodiments, a method of detecting the presence of hemorrhagic virus, e.g., an ebolavirus, in a sample, includes (a) contacting the sample with a sensor cell, e.g., sensor fungal cell, comprising a heterologous GPCR that binds to an analyte, e.g., peptide analyte, derived from the hemorrhagic virus, e.g., the ebolavirus, wherein the analyte is a ligand for the heterologous GPCR, (b) binding of the analyte present in the sample to the heterologous GPCR, wherein binding of the analyte, e.g., peptide analyte, to the heterologous GPCR triggers an appearance of a reporter, wherein the reporter is a visible light pigment, e.g., a biosynthesized visible light pigment and (c) detecting the appearance of the reporter by the naked eye, wherein the appearance of the reporter indicates the presence of the hemorrhagic virus, e.g., the ebolavirus, in the sample. In certain embodiments, the biosynthesized visible light pigment is lycopene.

In certain embodiments, the present disclosure provides methods for detecting a virus in a subject, e.g., by detecting the presence of a respiratory virus and/or a hemorrhagic virus, in a sample obtained from the subject. Non-limiting examples of clinically relevant samples are disclosed herein. In certain embodiments, the samples that can be analyzed by the disclosed methods include body fluid samples such as, but not limited to, intestinal fluids, diarrhea or other feces, mucus (e.g., sputum), blood, cerebrospinal fluid, synovial fluid, lymph, pus, saliva, vomit, urine, bile, vaginal fluid, bronchoalveolar lavage and sweat. In certain embodiments, the sample is an environmental sample such as, but not limited to, water, air and soil samples. In certain embodiments, the sample is a culture supernatant. In certain embodiments, the sample is a mucus sample. In certain embodiments, the sample is a blood sample. In certain embodiments, the sample is a mucus sample. In certain embodiments, the sample is obtained from a nasal swab.

### C. Methods of Detecting Protein Variants

The disclosed subject matter provides a method for detecting the presence of a polypeptide variant in a sample using one or more sensor cells disclosed herein. In certain embodiments, the present disclosure provides methods for detecting the presence of a protein variant (or fragment thereof) in a sample using one or more sensor cells disclosed herein.

In certain embodiments, the method can be a diagnostic method, where the determination of the presence of a protein variant provides a diagnosis for the subject from which the sample was obtained, e.g., subject can be treated.

In certain embodiments, a method of the present disclosure can be used to detect a binding event between a protein or polypeptide and a receptor. In certain embodiments, the protein or polypeptide can be an antibody. In certain embodiments, a method of the present disclosure can be used to detect a binding event between an antibody and variant thereof and a receptor.

In certain embodiments, a method of the present disclosure can be used to detect the presence of a variant of a protein that causes an infection, disease and/or disorder. For example, but not by way of limitation, the methods disclosed herein can be used to determine if a subject has a particular infection, disease and/or disorder based on the detection of a protein variant clinically relevant to the infection, disease and/or disorder. Non-limiting examples of infections, diseases and/or disorders that are caused by a variant of a protein are neurological disorders, blood disorders, e.g., sickle cell disease, cancers and viral diseases and/or infections. In certain embodiments, the virus is porcine epidemic diarrhea virus.

In certain embodiments, the present disclosure provides a method for detecting the presence of a variant of a protein from a virus, e.g., a respiratory virus, in a sample. In certain embodiments, the present disclosure provides a method for detecting the presence of a variant of a protein from SARS-CoV-2. In certain embodiments, the present disclosure provides a method for detecting the presence of a SARS-CoV-2 virus variant by detecting the presence of a protein variant specific to the SARS-CoV-2 virus variant in a sample. For example, but not by way of limitation, the present disclosure provides a method for detecting the presence of a SARS-CoV-2 virus variant by detecting the presence of a specific SARS-CoV-2 Spike protein mutation in a sample.

In certain embodiments, the method includes contacting the sample with a first sensor cell (e.g., a yeast sensor cell) expressing (1) a receptor (e.g., a heterologous GPCR receptor) that binds to one or more variants of a protein (or a fragment thereof) and the wild type protein (or a fragment thereof) and (2) a protease that specifically cleaves the one or more variants of the protein (or a fragment thereof). In certain embodiments, the method further includes contacting the sample with a second sensor cell (e.g., a yeast sensor cell) expressing a receptor (e.g., a heterologous GPCR receptor) that binds to the one or more variants of a protein (or a fragment thereof). In certain embodiments, the second cell does not express a protease. In certain embodiments, the method further includes comparing the activation of the receptor expressed by the first sensor cell to the activation of the receptor expressed by the second sensor cell. In certain embodiments, the protein variant is present in the sample when the receptor expressed by the first sensor cell, which expresses the protease, is not activated and the receptor expressed by the second sensor cell, which does not express the protease, is activated. In certain embodiments, the protein variant is not present in the sample when the receptor expressed by the first sensor cell, which expresses the protease, is activated and the receptor expressed by the second sensor cell, which does not express the protease, is also activated. In certain embodiments, the receptor in the first sensor cell and the receptor in the second sensor cell are the same.

The present disclosure provides methods for detecting the presence of one or more protein variants. For example, but not by way of limitation, the present disclosure provides a method for detecting the presence of a protein variant (or a fragment thereof), e.g., a protein variant clinically relevant to an infection, disease and/or disorder. In certain embodiments, the method can include (a) contacting a sample with a first sensor cell expressing a first heterologous receptor, e.g., GPCR, that binds to the wild type protein (or a fragment thereof) and the protein variant (or a fragment thereof) coupled to a reporter gene such that when the protein variant and/or wild type protein binds to the receptor, expression of the reporter gene is induced; (b) contacting a sample with a second sensor cell expressing (i) a second heterologous receptor, e.g., GPCR, that binds to the wild type protein and the protein variant coupled to a reporter gene such that when the protein variant and/or wild type protein binds to the receptor, expression of the reporter gene is induced and (ii) a protease that cleaves the protein variant; (c) culturing the first sensor cell and the second sensor cell for an effective period of time; and (d) determining whether the reporter has been produced in the first sensor and the second sensor cell. In certain embodiments, the first and second heterologous receptors are the same, e.g., have the same binding specificity. Alternatively, the first and second heterologous receptors are different but both receptors bind the protein variant and/or wild type protein. In certain embodiments, if the sample includes the protein variant than the receptor of the second cell will not be activated by the protein variant but the receptor of the first cell will be activated by the protein variant that has not been cleaved. In certain embodiments, determining whether the reporter is produced is performed by the naked eye and does not require instrumentation. In certain non-limiting embodiments, the reporter is lycopene.

In certain embodiments, a method of detecting the presence of a protein variant in a sample, includes (a) contacting the sample with a first sensor cell, e.g., a first sensor fungal cell, comprising a first heterologous receptor, e.g., GPCR, that binds to a protein variant (or a fragment thereof) and wild type protein (or a fragment thereof), wherein the protein variant and wild type protein is a ligand for the heterologous receptor, e.g., GPCR, and wherein binding of the protein variant to the heterologous receptor, e.g., GPCR, triggers an appearance of a reporter; (b) contacting the sample with a second sensor cell, e.g., a second sensor fungal cell, comprising a second heterologous receptor, e.g., GPCR, that binds to the protein variant and wild type protein, wherein the protein variant and wild type protein is a ligand for the heterologous receptor, e.g., GPCR, and wherein binding of the protein variant to the heterologous receptor, e.g., GPCR, triggers an appearance of a reporter, and a protease that cleaves the protein variant; and (c) detecting the appearance of the reporter in the first sensor cell and the second sensor cell by the naked eye. In certain embodiments, the first and second heterologous receptors are the same, e.g., have the same binding specificity. Alternatively, the first and second heterologous receptors are different but both receptors bind the protein variant and/or wild type protein. In certain embodiments, the appearance of the reporter in the first sensor cell and not in the second sensor cell indicates the presence of the protein variant in the sample. In certain embodiments, the appearance of the reporter in the first sensor cell and the second cell indicates that the protein variant is not present in the sample. In certain embodiments, the reporter is a visible light pigment, e.g., a biosynthesized visible light pigment. In certain embodiments, the biosynthesized visible light pigment is lycopene.

In certain embodiments, the first sensor cell can further express one or more additional receptors that specifically bind other variants of the protein, e.g., variants that have more than 2, more than 3, more than 4, more than 5 or more than 10 amino acid differences from the wild type protein. In certain embodiments, the second cell can further express one or more additional proteases that specifically cleave the other variants of the protein.

In certain embodiments, the method can include (a) contacting a sample with a first sensor cell expressing (i) a first heterologous receptor that binds to the wild type protein and the protein variant that is coupled to a reporter gene such that when the protein variant and/or wild type protein binds to the receptor, expression of the reporter gene is induced and (i) a first protease that cleaves the protein variant; (b) contacting a sample with a second sensor cell expressing (i) a second heterologous receptor that binds to the wild type protein and the protein variant that is coupled to a reporter gene such that when the protein variant and/or wild type protein binds to the receptor, expression of the reporter gene is induced and (ii) a first protease that cleaves the wild type protein; (c) contacting a sample with a third sensor cell expressing (i) a third heterologous receptor that binds to the wild type protein and the protein variant coupled to a reporter gene such that when the protein variant and/or wild type protein binds to the receptor, expression of the reporter gene is induced and (ii) a first protease that cleaves the protein variant and a second protease that cleaves the wild type protein; (d) culturing the first sensor cell, the second sensor cell and the third sensor cell for an effective period of time; and (e) determining whether the reporter has been produced in the first sensor cell, the second sensor cell and the third sensor cell. In certain embodiments, the first, second heterologous and third receptors are the same, e.g., have the same binding specificity. Alternatively, the first, second heterologous and third receptors are different but both receptors bind the protein variant and/or wild type protein. In certain embodiments, if the sample includes the protein variant than the receptor of the second cell will be activated by the protein variant but the receptor of the first cell and the second cell will not be activated by the protein variant that has been cleaved. In certain embodiments, if the sample includes the wild type protein than the receptor of the first cell will be activated by the wild type protein but the receptor of the second cell and the third cell will not be activated by the wild type protein that has been cleaved. In certain embodiments, determining whether the reporter is produced is performed by the naked eye and does not require instrumentation. In certain non-limiting embodiments, the reporter is lycopene.

In certain embodiments, the present disclosure provides methods for detecting the presence of one or more variants of an S protein of SARS-CoV-2. In certain embodiments, the method can include (a) contacting a sample with a first sensor cell expressing a first heterologous receptor, e.g., GPCR, that binds to an S protein variant (or a fragment thereof) and a wild type S protein (or other S protein variant (or a fragment thereof)) coupled to a reporter gene such that when the S protein variant and/or wild type S protein (or other S protein variant) binds to the receptor, expression of the reporter gene is induced; (b) contacting a sample with a second sensor cell expressing (i) a second heterologous receptor, e.g., GPCR, that binds to the S protein variant and a wild type S protein (or other S protein variant) coupled to a reporter gene such that when the S protein variant and/or wild type S protein (or other S protein variant) binds to the receptor, expression of the reporter gene is induced and (ii) a protease that cleaves the S protein variant; (c) culturing the first sensor cell and the second sensor cell for an effective period of time; and (d) determining whether the reporter has been produced in the first sensor and the second sensor cell. In certain embodiments, the first and second heterologous receptors are the same. Alternatively, the first and second heterologous receptors are different but both receptors bind the S protein variant and/or wild type S protein (or other S protein variant). In certain embodiments, if the reporter is detected in both the first sensor cell and the second cell then the S protein variant is not present in the sample. In certain embodiments, if the reporter is not detected in the second cell but is detected in the first sensor cell then the S protein variant is present in the sample as it was cleaved by the protease of the second cell. In certain embodiments, determining whether the reporter is produced is performed by the naked eye and does not require instrumentation. In certain non-limiting embodiments, the reporter is lycopene.

In certain embodiments, the first sensor cell can further express one or more additional proteases that specifically cleave a S protein variant (or a fragment thereof). In certain embodiments, a sensor cell can express (1) a protease that cleaves the delta variant of the S protein and (2) an additional protease that cleaves the beta variant of the S protein.

In certain embodiments, a method of detecting the presence of a variant of an S protein of SARS-CoV-2, in a sample, includes (a) contacting the sample with a first sensor cell, e.g., a first sensor fungal cell, comprising a first heterologous receptor, e.g., GPCR, that binds to a variant of an S protein of SARS-CoV-2 and wild type S protein (or other S protein variant), wherein the S protein variant and wild type S protein (or other S protein variant) are ligands for the heterologous receptor, e.g., GPCR, and wherein binding of the S protein variant and/or wild type S protein (or other S protein variant) to the heterologous receptor, e.g., GPCR, triggers an appearance of a reporter; (b) contacting the sample with a second sensor cell, e.g., a second sensor fungal cell, comprising a second heterologous receptor, e.g., GPCR, that binds to the variant of an S protein of SARS-CoV-2 and wild type S protein (or other S protein variant), wherein the S protein variant and wild type S protein (or other S protein variant) are ligands for the heterologous receptor, e.g., GPCR, and wherein binding of the S protein variant and/or wild type S protein (or other S protein variant) to the heterologous receptor, e.g., GPCR, triggers an appearance of a reporter, and a protease that cleaves the variant of an S protein of SARS-CoV-2; and (c) detecting the appearance of the reporter in the first sensor cell and the second sensor cell by the naked eye. In certain embodiments, the appearance of the reporter in the first sensor cell and not in the second sensor cell indicates the presence of the variant of an S protein of SARS-CoV-2 in the sample. In certain embodiments, the appearance of the reporter in the first sensor cell and the second cell indicates that the variant of an S protein of SARS-CoV-2 is not present in the sample. In certain embodiments, the reporter is a visible light pigment, e.g., a biosynthesized visible light pigment. In certain embodiments, the biosynthesized visible light pigment is lycopene.

In certain embodiments, the methods disclosed herein can be used to detect one or more of the following SARS-CoV-2 variants: the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants.

In certain embodiments, the present disclosure provides methods for detecting a protein variant in a sample obtained from the subject. Non-limiting examples of clinically relevant samples are disclosed herein. In certain embodiments, the samples that can be analyzed by the disclosed methods include body fluid samples such as, but not limited to, intestinal fluids, diarrhea or other feces, mucus (e.g., sputum), blood, cerebrospinal fluid, synovial fluid, lymph, pus, saliva, vomit, urine, bile, vaginal fluid, bronchoalveolar lavage and sweat. In certain embodiments, the sample is an environmental sample such as, but not limited to, water, air and soil samples. In certain embodiments, the sample is a culture supernatant. In certain embodiments, the sample is a mucus sample. In certain embodiments, the sample is a blood sample. In certain embodiments, the sample is a mucus sample. In certain embodiments, the sample is obtained from a nasal swab.

In certain embodiments, the protease and/or proteolytic agent is added to the sample to be analyzed before, during and/or after contacting the sample with a sensor cell.

### IX. Kits and Products

The present disclosure provides kits and products for detecting an analyte described herein. For example, but not by way of limitation, a kit and/or product of the present disclosure can include one or more sensor cells, as described above, that can be used to perform methods of detecting the presence of an analyte, as described above. In certain embodiments, the sensor cell can be freeze-dried or lyophilized. In certain embodiments, the kit can further include a food source, e.g., medium, sugar or agar, for activating the sensor cell.

In certain embodiments, a kit and/or product can further include one or more controls. For example, but not by way of limitation, kits and/or products can include both a positive and a negative control.

In certain embodiments, a kit and/or product of the present disclosure can further include a substrate on which or in which detection can occur, e.g., a dish, cup, bowl, plate, paper, chip, gel, bag, stick, syringe, test tube, jar or bottle, and that comprises one or more sensor cells. In certain embodiments, a kit and/or product of the present disclosure can include one or more test tubes that comprises a plurality of sensor cells. In certain embodiments, a kit or product of the present disclosure can include a substrate, e.g., paper, that has sensor cells present on the substrate, e.g., in a circular pattern on the substrate.

In certain embodiments, a kit and/or product of the present disclosure can include one or more devices and/or materials for collecting a sample for analysis. For example, but not by way of limitation, a device and/or material for collecting a sample for analysis can include a swab, a pipette, a dropper and/or a cell scraper. In certain embodiments, a kit and/or product of the present disclosure can include a swab, e.g., for collecting a mucosal sample.

In certain embodiments, a kit and/or product of the present disclosure can include a food or nutrient source, e.g., sugar or agar, for the one or more sensor cells. In certain embodiments, a kit and/or product of the present disclosure can include components to improve cell viability, including one or more carbon sources, one or more nitrogen sources, one or more trace nutrient sources, and one or more additional nutrient sources to improve response speed.

In certain embodiments, a kit and/or product of the present disclosure can include additional assay components, including dyes, filters and/or cryo-protectants.

In certain embodiments, a kit and/or product of the present disclosure can be produced by combining all required assay components (e.g., nutrients, sensor cells and proteases) and freeze-drying, air-drying, or binding this component mix to a substrate.

In certain embodiments, the kit and/or product comprises a protease (e.g., a protease from prokaryote sources or a protease from eukaryote sources) for digestion of proteins of interest into smaller detectable peptides. Non-limiting examples of proteases are provided in Section V and the examples herein. For example, but not way of limitation, the protease can be an endoproteinase. In certain embodiments, the proteinase can be an Arg-C, Asp-N, rAsp-N, Chymotrypsin, Glu-C, rLys-C, Endoproteinase Lys-C, Endoproteinase Lys-N, Elastase, Pepsin and/or Thermolysin. In certain embodiments, the proteinase is Arg-C proteinase.

### A. Kits and Products for Detecting Fungal Species

The disclosed subject matter provides kits and/or products for detecting the presence of a fungal species disclosed herein.

In certain embodiments, the present disclosure provides for kits and/or products for detecting an analyte derived from a fungal species that causes aspergillosis, blastomycosis, coccidiodmycosis, pneumocystis and/or candidiasis.

In certain embodiments, the present disclosure provides for kits and/or products for detecting an analyte derived from a species from the Aspergillus genus. In certain embodiments, the species of the Aspergillus genus is selected from A. nidulans, A. fumigatus, A. terreus, A. flavus, A. niger, A. clawztus, A. oryzae, A. novofumigatus, A. lentulus, A. viridinutans, A. udagawae and/or N. fischeri.

In certain embodiments, the presently disclosed subject matter provides kits and/or products for detecting the presence of fungal species selected from A. nidulans, A. fumigatus, A. terreus, A. flavus, A. niger, A. clavatus, A. oryzae, A. novofumigatus, A. lentulus, A. viridinutans, A. udagawae, N fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marneffei. P. carinii, P. murina, P. wakefieldiae, C. dubliniensis, C. auris, C. pseudohaemulonii, C. haemuloni, C. duobushaemulonis, C. metapsilosis and/or C. orthopsilosis.

In certain embodiments, a kit and/or product of the present disclosure can be used for detecting the presence of a species of the Aspergillus genus, e.g., A. fumigatus.

In certain embodiments, a kit and/or product of the present disclosure can include one or more sensor cells, as described above, that can be used to perform methods of detecting the presence of a fungal species.

In certain embodiments, a kit and/or product can further include one or more controls. For example, but not by way of limitation, a kit and/or product can include a sample that includes a fungal species disclosed herein, e.g., a species of the Aspergillus genus, e.g., A. fumigatus, as a positive control. In certain embodiments, a kit can include a sample that does not include a fungal species disclosed herein, e.g., a species of the Aspergillus genus, e.g., A. fumigatus, as a negative control.

In certain embodiments, the kit and/or product comprises a protease (e.g., a protease from prokaryote sources or a protease from eukaryote sources) for digestion of the fungal species, e.g., a species of the Aspergillus genus, e.g., A. fumigatus, into smaller detectable peptides.

### B. Kits and Products for Detecting Viruses

The disclosed subject matter provides kits and products for detecting the presence of a virus disclosed herein. In certain embodiments, the presently disclosed subject matter provides kits for detecting the presence of virus selected from a respiratory virus, a hemorrhagic virus, a gastrointestinal virus, a sexually transmitted virus, an exanthematous virus, a hepatitis virus and/or a neurological virus.

In certain embodiments, a kit or product of the present disclosure can be used for detecting the presence of a respiratory virus, e.g., a coronavirus (e.g., SARS-CoV-2).

In certain embodiments, a kit or product of the present disclosure can be used for detecting the presence of a hemorrhagic virus, e.g., an ebolavirus.

In certain embodiments, a kit or product of the present disclosure can be used for detecting the presence of a gastrointestinal virus.

In certain embodiments, a kit or product of the present disclosure can be used for detecting the presence of an exanthematous virus

In certain embodiments, a kit or product of the present disclosure can be used for detecting the presence of a hepatitis virus.

In certain embodiments, a kit or product of the present disclosure can be used for detecting the presence of a neurological virus.

In certain embodiments, a kit or product of the present disclosure can be used for detecting the presence of a sexually transmitted virus.

In certain embodiments, a kit or product of the present disclosure can include one or more sensor cells, as described above, that can be used to perform methods of detecting the presence of an analyte, as described above. In certain embodiments, a kit or product of the present disclosure can include one or more sensor cells, as described above, that can be used to perform methods of detecting the presence of an analyte derived from a respiratory virus, e.g., a coronavirus (e.g., SARS-CoV-2). In certain embodiments, a kit or product of the present disclosure can include one or more sensor cells, as described above, that can be used to perform methods of detecting the presence of an analyte derived from a hemorrhagic virus, e.g., an ebolavirus.

In certain embodiments, a kit or product can further include one or more controls. Kits can include both a positive and a negative control. For example, but not by way of limitation, a kit can include a sample that includes a virus, e.g., an inactivated virus, disclosed herein, e.g., a respiratory virus, a hemorrhagic virus, a gastrointestinal virus, an exanthematous virus, a hepatitis virus and/or a neurological virus, as a positive control. In certain embodiments, a kit or product can include a sample that does not include a virus disclosed herein, e.g., a respiratory virus, a hemorrhagic virus, a gastrointestinal virus, an exanthematous virus, a hepatitis virus and/or a neurological virus, as a negative control.

In certain embodiments, the kit or product comprises a protease (e.g., a protease from prokaryote sources or a protease from eukaryote sources) for digestion of proteins of the virus into smaller detectable peptides.

### C. Kits and Products Detecting Protein Variants

The disclosed subject matter provides kits and/or products for detecting the presence of a protein variant. For example, but not by way of limitation, a kit and/or product of the present disclosure can be used to determine whether a protein variant is present in a sample from a subject, e.g., a biological sample from a subject.

In certain embodiments, a kit or product of the present disclosure can be used to detect a protein and a variant of the protein that is clinically relevant to an infection, disease and/or disorder.

In certain embodiments, a kit or product of the present disclosure can be used to detect a protein and/or a variant thereof derived from a virus, e.g., a coronavirus. In certain embodiments, a kit or product of the present disclosure can be used to detect an S protein variant and/or the wild type form of the S protein. In certain embodiments, a kit or product of the present disclosure can be used to detect two or more variants of the S protein. In certain embodiments, a kit and/or product of the present disclosure can be used for detecting an S protein variant of a SARS-CoV-2 variant. In certain embodiments, the kit and/or product can be used to detect an alpha variant, a beta variant, a delta variant, a gamma variant, an epsilon variant, a kappa variant, an iota variant, an eta variant, a lambda variant, a mu variant, a zeta variant or an omicron variant of SARS-CoV-2. In certain embodiments, the kit or product can be used to detect one or more of the following SARS-CoV-2 variants: the B.1.1.7 (alpha), B.1.351 (beta), P.1 (gamma), B.1.427 (epsilon), B.1.429 (epsilon), B.1.617.1 (kappa), B.1.617.2 (delta), B.1.526.1 (iota), B.1.526.2 (iota), B.1.525 (eta), P.2 (zeta), C.37 (lambda), B.1.621 (mu), B.1.1.529 (omicron), BA.1 (omicron), BA.1.1 (omicron), BA.2 (omicron), BA.3 (omicron), BA.4 (omicron), BA.5 (omicron) and/or B.1.526 (iota) variants.

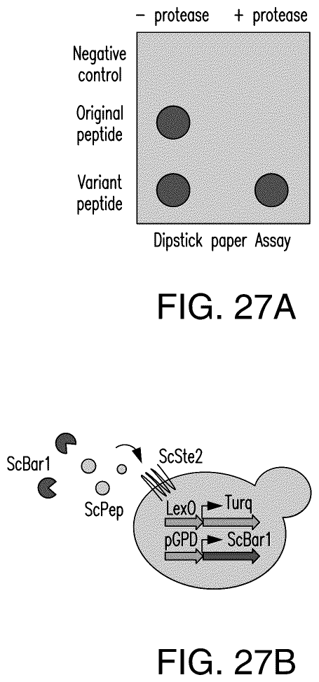

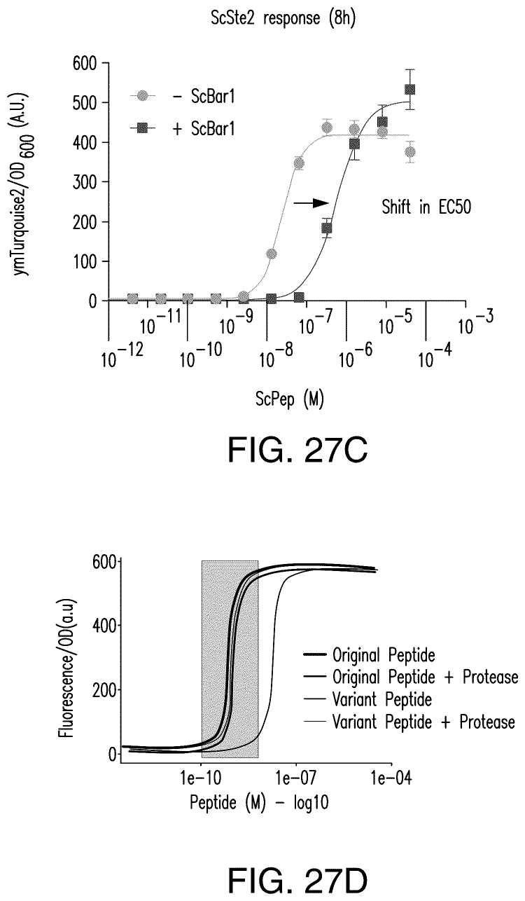

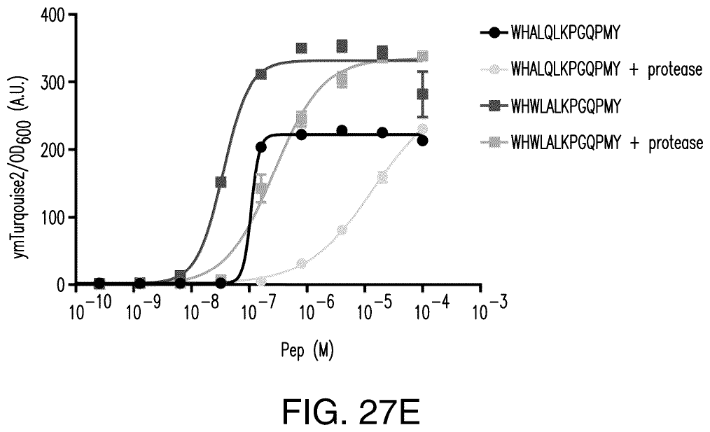

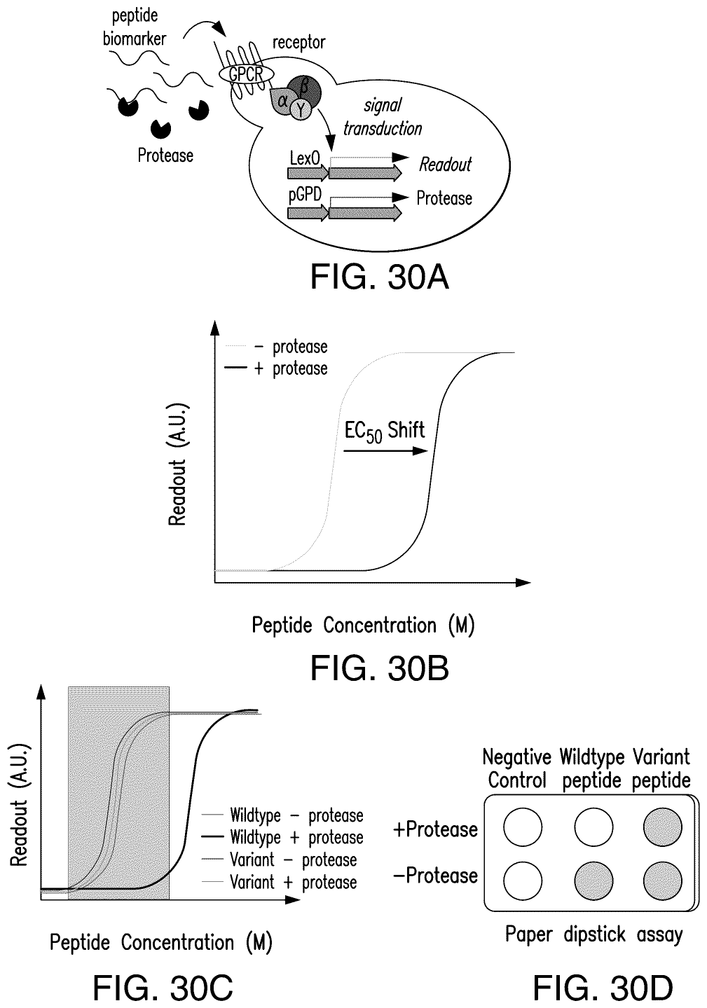

In certain embodiments, a kit and/or product of the present disclosure can include one or more sensor cells, as described above, that can be used to perform methods of detecting the presence of a protein variant and/or wild type form of the protein. In certain embodiments, a kit and/or product of the present disclosure can include a first sensor that expresses a first protease that specifically cleaves a first variant of a protein. In certain embodiments, a kit and/or product of the present disclosure can include a second sensor cell that expresses a first protease that specifically cleaves the wild type protein. In certain embodiments, a kit and/or product of the present disclosure can include a third sensor cell that expresses a first protease that specifically cleaves a first variant of a protein and can express a second protease that specifically cleaves the wild type protein. In certain embodiments, each of these sensor cells can be present on a substrate, e.g., at different places on the substrate, as shown in FIG. 27A and FIG. 30D.

In certain embodiments, a kit and/or product can further include one or more controls. For example, but not by way of limitation, a kit and/or product can include a sample that includes a protein variant, e.g., an S protein variant, as a positive control. In certain embodiments, a kit and/or product can include a sample that does not include a protein variant, e.g., an S protein variant, as a negative control.

In certain embodiments, the kit and/or product comprises a protease (e.g., a protease from prokaryote sources or a protease from eukaryote sources) for digestion of the protein variants or wild type proteins of interest.

### EXAMPLES

The presently disclosed subject matter will be better understood by reference to the following Examples, which are provided as exemplary of the presently disclosed subject matter, and not by way of limitation.

### Example 1: Development of a Live Yeast Biosensor for A. fumigatus

Fungal diseases are a persistent global health problem with an estimated 1.5 million annual deaths worldwide. Unfortunately, public health surveillance of fungal diseases are not well established, suggesting that the actual death toll could be significantly higher. Moreover, there is an increasing number of susceptible populations to fungal diseases, such as patients with asthma, chronic obstructive pulmonary disease, tuberculosis, cancer, immunocompromised, and more recently, coronavirus disease 2019 (COVID-19). With the growing incidence of the infections, timely and accurate diagnosis has become paramount in order to start early treatment with antifungal therapy which substantially improves patient clinical outcomes.

One of the most common fungal genera to cause invasive fungal infections is Aspergillus. Within this genus, A. fumigatus is the most common species that causes invasive aspergillosis and allergic disease. A. fumigatus is a ubiquitous saprophyte present in air and soil globally due to its ability to grow at a wide range of temperatures and pH. A. fumigatus is an opportunistic fungal pathogen and does not typically colonize human respiratory tract. However, continuous exposure to the fungus can lead to invasive infections in people with compromised immune system with a mortality rate ranging from 30% to 95% and emerging as one of the most common causes of infection-related deaths. The most important predictors of survival from invasive aspergillosis are early diagnosis and immediate start of appropriate antifungal therapy. Currently, the diagnosis of invasive aspergillosis is challenging, time consuming, and requires a combination of clinical, radiological and microbiological techniques. Clinical and radiographical diagnosis is established late in the course of the disease, where invasive fungal growth leads to symptoms like fever, cough, pleuritic chest pain, pneumonia and lung cavitation. Microbiological methods have proven to be more promising in accurate detection of aspergillosis with a few detection approaches that are being used so far: molecular techniques and detection of cell wall polysaccharides. Molecular detection of Aspergillus is not well established due to lack of standardization and variable performances in different settings. The standard method for diagnosing aspergillosis is the detection of the cell wall component galactomannan (GM), a polysaccharide that is primarily present in the cell wall of Aspergillus spp. This Platelia™ Test (BioRad, Marnesla-Coquette, France) is an immunoenzymatic sandwich microplate assay that uses rat monoclonal antibody that detects GM antigen in human serum and bronchoalveolar fluid (BALF) with 82% sensitivity and 81% specificity in neutropenic patients. More recently, the Sona Aspergillus GM Lateral flow assay (GM-LFA) rapid test has been commercialized with overall 77% sensitivity and 81% specificity. However, galactomannan testing remains limited due to cost, turnaround time and variable performance in different patient populations.

This example discloses the development and validation of a live-yeast-based assay for detection of A. fumigatus from culture supernatants. This assay is based on the detection of A. fumigatus mating pheromone through heterologously expressed GPCR in Saccharomyces cerevisiae, which then transcriptionally activates the biosynthesis of a red pigment (lycopene) visible to the naked eye. An exemplary schematic of a living yeast biosensor for detection of A. fumigatus is shown in FIG. 1A.

### Materials and Methods

Bacterial Strains and Growth Media: NEB® C3040 E. coli was used for all cloning experiments. Selection and growth of E. coli was performed in Luria Broth (LB) medium at 37° C. with aeration. With the exception of generating competent cells, the LB medium was supplemented with appropriate antibiotics (ampicillin 100 μg/mL, or chloramphenicol 34 μg/mL).

Yeast Strains and Growth Media: All strains used in this example are a derivative of the yTC370 parent strain (ste2Δ0 ura3Δ0 sfGFPΔ0 leu2Δ0), which is itself a derivative of yWS890 (pCCW12-STE2-tSSA1-pPGK1-GPA1-tENO2-pRAD27-LexA-PRD-tENO1-URA3, LexO(6×)-pLEU2m-sfGFP-tTDH1-LEU2). yWS890 is itself a derivative of yWS677 (sst2Δ0 far1Δ0 bar1Δ0 ste2Δ0 ste12Δ0 gpa1Δ0 ste3Δ0 mf(alpha)1Δ0 mf(alpha)2Δ0 mfa1Δ0 mfa2Δ0 gpr1Δ0 gpa2Δ0), and yWS677 is a derivative of BY4741 (MATα his3Δ1 leu2Δ0 met15Δ0 ura3Δ0). yWS890 and yWS677 were developed as previously described (Shaw et al. Engineering a Model Cell for Rational Tuning of GPCR Signaling. Cell 177, 1-15 (2019)). The yTC370 parent strain was generated using CRISPR/Cas9 genome engineering. Further editing of yTC370 to create the additional strains was performed in a single procedure using CRISPR/Cas9 genome engineering.

Yeast extract peptone dextrose (YPD) was used for culturing cells in preparation for transformation: 1% (w/v) Bacto Yeast Extract (Fisher), 2% (w/v) Bacto Peptone (Fisher), 2% glucose (Fisher). Cells were cultured at 30° C. with shaking at 200 rpm.

Selection of yeast transformants was performed on synthetic complete (SC) dropout agar medium: 2% (w/v) glucose (Fisher), 0.67% (w/v) Yeast Nitrogen Base without amino acids (Sigma), 0.14% (w/v) Yeast Synthetic Drop-out Medium Supplements without histidine, leucine, tryptophan, and uracil (Sigma) supplemented with 20 mg/L tryptophan (Sigma), and 20 g/L bacteriological agar (Fisher). Depending on the required selection, SC dropout media was supplemented with 20 mg/L uracil (Sigma), 100 mg/L leucine (Sigma), and 20 mg/L histidine (Sigma). Cells were grown at 30° C. static. Agar was added to 2% for preparing solid yeast media.

All liquid experiments were performed in synthetic complete (SC) medium with 2% (w/v) glucose (VWR), 0.67% (w/v) Yeast Nitrogen Base without amino acids (Sigma), 0.14% (w/v) Yeast Synthetic Drop-out Medium Supplements without histidine, leucine, tryptophan, and uracil (Sigma), 20 mg/L uracil (Sigma), 100 mg/L leucine (Sigma), 20 mg/L histidine (Sigma), and 20 mg/mL tryptophan (Sigma) unless otherwise stated. Unless otherwise stated, all yeast strains were cultured in 200 μL of SC medium and grown in 200-μL 96-well plates at 30° C. in a high-frequency shaker, shaking at 800 rpm.

Bacterial Transformations: Electrocompetent cells were created. C3040 E. coli from the −80° C. glycerol stock was streaked out on LB plate and grown overnight. A single colony was picked and grown for 14-16 hours in 2 mL of LB medium. Then the whole culture was added to 2 L of low-salt SOB containing 2% (w/v) tryptone, 0.5% (w/v) Bacto Yeast Extract and 0.05% (w/v) sodium chloride. The culture was grown for 4-5 h to OD₆₀₀˜0.6-0.8, split between four 500 mL centrifuge-safe bottles, and centrifuged at 6,000 rpm at 4° C. for 10 min. The supernatant was then discarded, and the cell pellets resuspended by aspiration in ice-cold 10% (v/v) glycerol. The cells were pelleted again, the supernatant was discarded, the cells were resuspended in glycerol, unified in two bottles, and centrifuged 6,300 rpm at 4° C. for 15 min. Previous procedures were repeated two more times with final unification in a single bottle and final resuspension in 3 mL of ice-cold 10% (v/v) glycerol. 100 μL of the cell suspension was aliquoted into 1.5-mL Eppendorf tubes, flash frozen on dry ice, and put into −80° C. freezer for long term storage. To transform the DNA, 50 μL of the electrocompetent cells were thawed on ice and 5 μL of 1:3 water dilution of DNA was added to the cells. Cell/DNA mixture was transferred to 0.1-cm gap electroporation cuvette (Bio-Rad) and one 1.8 kV pulse was given. The cells were recovered in 1 mL LB medium at 37° C. for 30-45 min. Cells were then plated on solid LB medium supplemented with the appropriate antibiotics.

Yeast Transformations: Chemically competent yeast cells were created following the lithium acetate method (Daniel Gietz, R & Schiestl, R. H. High-efficiency yeast transformation using the LiAc/SS carrier DNA/PEG method. Nat. Proto. 2, 31-34 (2007)). Yeast colonies were grown to saturation overnight in YPD. The following morning the cells were diluted 1:100 in 5 mL of fresh YPD in a 15-mL culture tube and grown for 4-6 h to OD₆₀₀ 0.8-1.0. Cells were pelleted and washed once with 5 mL 0.1 M lithium acetate (LiOAc) (Sigma). Cells were then resuspended in 0.1 M LiOAc to a total volume of 100 mL/transformation. 100 mL of cell suspension was then distributed into 1.5 mL reaction tubes and pelleted. Cells were resuspended in 64 μL of DNA/salmon sperm DNA mixture (10 μL of boiled salmon sperm DNA (Fisher)+DNA+ddH₂O), and then mixed with 294 μL of PEG/LiOAc mixture (260 μL 50% (w/v) PEG-3350 (Sigma)+36 μL 1 M LiOAc). The yeast transformation mixture was then heat-shocked at 42° C. for 40 mins, pelleted, resuspended in 150 μL 5 mM CaCl₂ (Sigma) and plated onto the appropriate synthetic dropout medium.

Construction of Communication Part Plasmids and Yeast Genomic Integration Plasmid: The communication part plasmids are based on pYTK009 (ori, CmR selection marker). Part plasmids having individual expression cassettes with biosensor parts (constitutive GPCR expression, pheromone inducible fluorescent protein expression, pheromone inducible lycopene biosynthetic enzyme expression, pheromone inducible peptide expression and constitutive protease expression) were cloned using Gibson Assembly. Yeast genomic integration plasmid is an acceptor plasmid (ori, AmpR selection marker) into which biosensor component part plasmids are being assembled via Golden Gate Assembly. The integration plasmid contains appropriate 500-bp homology sequences for genomic integration (ARS208a, HO or LEU2), integration selection marker (LEU2, HIS3 or URA3), RFP which gets replaced with correctly assembled biosensor parts and unique NotI restriction sites for linearization.

Golden Gate Assembly of Biosensor Parts into Yeast Genomic Integration Plasmid: A Golden Gate reaction mixture was prepared as follows: appropriate volume of each DNA plasmid in 1:1 ratio with integration plasmid set to 10 ng, 0.75 μL T4 DNA Ligase buffer (NEB), 0.75 μL T4 DNA Ligase (NEB), 0.45 μL BsaI restriction enzyme, 0.075 μL BSA and water to bring the final volume to 7.5 μL. Reaction mixtures were incubated in a thermocycler according to the following program: 50 cycles of digestion and ligation (37° C. for 3 min, 16° C. for 4 min) followed by a final digestion step (37° C. for 30 min), and a heat inactivation step (80° C. for 5 min). The mixture is then diluted 1:3 with water and E. coli is transformed via electroporation. Correctly assembled plasmids are screened and verified using colony PCR and Sanger sequencing.

CRISPR/Cas9 System and Genomic Integration: In a typical genomic integration of assembled integration plasmids using CRISPR/Cas9, the plasmids were digested with NotI restriction enzyme and directly used along with Cas9/gRNA plasmid (URA3 or NAT selection marker) following lithium acetate yeast transformation method. Transformed colonies are grown on selective media for the selection marker integrated along with the remaining biosensor components and for the Cas9/gRNA plasmid. Correctly integrated colonies are screened phenotypically by activating integrated GPCR with appropriate synthetic peptide and sequence is verified using PCR amplification and Sanger sequencing from purified genomic DNA of the screened colony.

Bioinformatic extraction of A. fumigatus mating GPCR (AfuSte2) and peptide ligand (AfuPep): The MAT1-1 gene DNA sequence was selected from AspGD database for the A. fumigatus strain Af293. The amino acid sequence was identified through UniProt (Q4WYU8). Amino acid and nucleotide sequences used in this example are provided below.

A. fumigatus Ste2 nucleotide sequence:
(SEQ ID NO: 45)
ATGAACTCGACATTTGATCCTTGGACGCAGAATATCACTCTGACTCAGTCCGATGGGACGACGGT
CGTCTCATCTCTAGCACTAGCAGACGACTATCTGCACTACATGATTCGTCTCGGCATCAACTATG
GCGCTCAGCTCGGCGCATGCGCCGTACTCTTCCTAGTCCTCTTGCTGCTGACAAGACCAGAGAAA
CGCGTCTCATCGGTCTTTGTCCTGAACGTCTCTGCGTTACTTGCCAACATTATACGTCTGGGATG
CCAGCTCAGCTATTTCTCTACCGGATTCGCTAGGATGTATGCGTTGCTGGCTGGCGACTTCTCCA
GAGTGTCACGCGGTGCTTATGCCGGCCAAGTCATGGCATCTGTGTTCTTCACTATAGTCCTTATC
TGTGTGGAGGCCTCCTTGGTGCTTCAGGTGCAGGTTGTCTGTTCCAATCTCCTTCGTCAGTATCG
GATCCTGCTACTTGGTGCCTCGACGCTCGCTGCCCTTGTTCCGATCGGGGTTCGCTTGACTTACT
CGGTGATGAACTGCATGGTGATCATGCACGCAGGAACCATGGACCATCTGGATTGGCTAGAGAGT
GCCACGAATATCGTGACAACCGTCAGCATTTGCTTTTTCTGCGCCGTCTTCGTCGTCAAGCTTGG
ACTCGCCATCAAAATGCGAAAGAGATTGGGTGTGAAAAAATTCGGTCCGATGAGGGTCATCTTTA
TCATGGGTTGCCAAACTATGACCATCCCAGCCGTTTTCGCCATTTGCCAGTACTTCAGCAGAATT
CCAGAATTCTCGCATAACGTCCTAACCCTCGTCATCATATCGCTCCCTCTTTCCTCCATTTGGGC
TGGATTCGCCCTTGACCAGGCAAACTCCACAGCGCGCTCTACCGAGTCTCGTCACCATCTGTGGA
ACATACTCTCCTCTGACGGCGCCACGAGAGACAAGCCCAGCCAATGTGCCAACTCCCCCATGACT
TCTCCGAACACCACATGCTACTCAGAGCAGTCAACCAGTAAACCCCAGCAGGATCCGGAGAACGG
GTTCGGCATTTCGGTGGCGCATGACATCTCCATCCACAGCTTCCAAAAGGGTGGCCACGGGGATA
TT
Codon-optimized A. fumigatus Ste2 nucleotide sequence
without internal BsaI restriction sites:
(SEQ ID NO: 46)
ATGAACTCAACATTTGACCCCTGGACACAAAACATTACACTAACACAAAGTGACGGGACTACAGT
CGTCAGTTCACTTGCACTGGCTGATGACTACCTGCATTACATGATCAGGCTAGGAATTAACTATG
GGGCACAGTTAGGCGCCTGCGCCGTACTATTCCTTGTCCTACTTTTGCTTACCAGGCCCGAAAAG
AGGGTATCTAGTGTGTTCGTTTTGAACGTGAGTGCACTTTTAGCCAATATAATAAGGCTAGGTTG
TCAACTTTCCTACTTTTCCACGGGCTTCGCGCGTATGTACGCATTGTTGGCCGGTGACTTCAGCA
GAGTGAGCCGTGGAGCCTATGCGGGTCAAGTAATGGCTTCAGTATTTTTTACAATAGTTCTGATT
TGCGTCGAAGCCTCTTTAGTCCTGCAAGTGCAGGTCGTGTGCTCCAATCTTCTGCGTCAGTATAG
GATTCTTCTATTGGGCGCTTCCACCCTGGCAGCTCTGGTGCCCATAGGCGTACGTCTAACATACA
GTGTGATGAATTGCATGGTGATCATGCACGCTGGCACAATGGACCATCTGGACTGGTTAGAGAGC
GCAACAAATATCGTTACGACTGTCAGTATTTGTTTCTTTTGCGCGGTGTTCGTAGTGAAGTTGGG
CCTGGCTATAAAAATGAGGAAGAGATTGGGTGTCAAGAAGTTTGGACCGATGAGAGTAATATTCA
TCATGGGCTGCCAAACTATGACCATCCCTGCCGTGTTCGCAATCTGTCAGTATTTCAGTAGGATT
CCAGAATTCTCTCATAACGTTTTGACCTTAGTAATCATTTCCCTGCCCTTATCAAGTATATGGGC
CGGGTTCGCTTTGGATCAAGCGAACTCTACGGCTCGTAGTACAGAATCTCGTCACCACCTGTGGA
ATATTCTTTCATCAGATGGTGCGACAAGAGATAAACCTTCCCAGTGTGCAAATAGTCCAATGACA
AGTCCGAATACGACCTGTTATTCCGAACAGTCAACGTCCAAGCCTCAGCAAGATCCCGAGAACGG
GTTTGGGATTAGCGTAGCTCATGATATCAGTATCCACTCCTTCCAAAAAGGGGGGCACGGCGACA
TT
A. fumigatus Ste2 amino acid sequence encoded
by SEQ ID NO: 1 and 2:
(SEQ ID NO: 10)
MNSTFDPWTQNITLTQSDGTTVVSSLALADDYLHYMIRLGINYGAQLGACAVLFLVLLLLTRPEK
RVSSVFVLNVSALLANIIRLGCQLSYFSTGFARMYALLAGDESRVSRGAYAGQVMASVFFTIVLI
CVEASLVLQVQVVCSNLLRQYRILLLGASTLAALVPIGVRLTYSVMNCMVIMHAGTMDHLDWLES
ATNIVTTVSICFFCAVFVVKLGLAIKMRKRLGVKKFGPMRVIFIMGCQTMTIPAVFAICQYFSRI
PEFSHNVLTLVIISLPLSSIWAGFALDQANSTARSTESRHHLWNILSSDGATRDKPSQCANSPMT
SPNTTCYSEQSTSKPQQDPENGEGISVAHDISIHSFQKGGHGDI
A. fumigatus mating pheromone peptide:
(SEQ ID NO: 3)
WCHLPGQGC
A. fumigatus PpgA ORF AFUA 6G06360 (putative
pheromone peptide in italics);
(SEQ ID NO: 47)
ATGAAACTCCTCTCACTTGTTCTTGCTTCCTTCGCTGCCGCTGCTGTCCAGGCACACATTACGCC
GTGGTGCCATCTTCCTGGTCAGGGTTGCTACATGCTTAAGCGCGCTGCTGACGCCTCGGATGAAG
TTAGACGTTCAGCCAGTGCCGTCGCTGAGGCCGTTGCGGAAGCCTTCCCGCAGAGTCCGTGGTGC
CATCTTCCCGGTCAGGGTTGCGCCAAGGCGAAGAGAGCCGCCGAGGCCGCTGAAGAGGTGAAGCG
CTCTGCCGATGCCTTTGCTGAAGCCATGGCTGCTTTTGAGAAGGAATAG
Native MFalphal ORF (SGD:S000006108) - prepro-
sequence with ScPep (italics):
(SEQ ID NO: 48)
ATGAGATTTCCTTCAATTTTTACTGCAGTTTTATTCGCAGCATCCTCCGCATTAGCTGCTCCAGT
CAACACTACAACAGAAGATGAAACGGCACAAATTCCGGCTGAAGCTGTCATCGGTTACTTAGATT
TAGAAGGGGATTTCGATGTTGCTGTTTTGCCATTTTCCAACAGCACAAATAACGGGTTATTGTTT
ATAAATACTACTATTGCCAGCATTGCTGCTAAAGAAGAAGGGGTATCTTTGGATAAAAGAGAGGC
TGAAGCTTGGCATTGGTTGCAACTAAAACCTGGCCAACCAATGTACAAGAGAGAAGCCGAAGCTG
AAGCTTGGCATTGGCTGCAACTAAAGCCTGGCCAACCAATGTACAAAAGAGAAGCCGACGCTGAA
GCTTGGCATTGGCTGCAACTAAAGCCTGGCCAACCAATGTACAAAAGAGAAGCCGACGCTGAAGC
TTGGCATTGGTTGCAGTTAAAACCCGGCCAACCAATGTAC

A. fumigatus biosensor strain and signal amplification strain engineering with fluorescent readout: Strain yTC522 was constructed with CRISPR/Cas9 genome integration of constitutive AfuSte2 and inducible mCherry expression into ARS208a locus. Strain yTC523 was constructed with CRISPR/Cas9 genome integration of constitutive AfuSte2, inducible mCherry expression and inducible AfuPep secretion into ARS208a locus. Strain yTC524 was constructed with CRISPR/Cas9 genome integration of constitutive AfuSte2, inducible mCherry expression and inducible ScPep secretion into ARS208a locus. Strain yTC525 was constructed with CRISPR/Cas9 genome integration of constitutive ScSte2, intermediate constitutive expression of ScBar1, inducible ymTurquoise2 expression and inducible ScPep secretion into ARS208a locus. Strain yTC526 was constructed with CRISPR/Cas9 genome integration of constitutive ScSte2, low constitutive expression of ScBar1, inducible ymTurquoise2 expression and inducible ScPep secretion into ARS208a locus.

mCherry (RFP) sequence:
ATGGTTTCAAAAGGTGAAGAAGATAATATGGCTATTATTAAAGAATTTATGAGATTTAAAGTTCA
TATGGAAGGTTCAGTTAATGGTCATGAATTTGAAATTGAAGGTGAAGGTGAAGGTAGACCATATG
AAGGTACTCAAACTGCTAAATTGAAAGTTACTAAAGGTGGTCCATTACCATTTGCTTGGGATATT
TTGTCACCACAATTTATGTATGGTTCAAAAGCTTATGTTAAACATCCAGCTGATATTCCAGATTA
TTTAAAATTGTCATTTCCAGAAGGTTTTAAATGGGAAAGAGTTATGAATTTTGAAGATGGTGGTG
TTGTTACTGTTACTCAAGATTCATCATTACAAGATGGTGAATTTATTTATAAAGTTAAATTGAGA
GGTACTAATTTTCCATCAGATGGTCCAGTTATGCAAAAAAAAACTATGGGTTGGGAAGCTTCATC
AGAAAGAATGTATCCAGAAGATGGTGCTTTAAAAGGTGAAATTAAACAAAGATTGAAATTAAAAG
ATGGTGGTCATTATGATGCTGAAGTTAAAACTACTTATAAAGCTAAAAAACCAGTTCAATTACCA
GGTGCTTATAATGTTAATATTAAATTGGATATTACTTCACATAATGAAGATTATACTATTGTTGA
ACAATATGAAAGAGCTGAAGGTAGACATTCAACTGGTGGTATGGATGAATTATATAAATAG
ymTurquoise 2 sequence:
ATGGTTAGTAAAGGTGAAGAATTGTTCACTGGTGTAGTGCCAATTTTAGTAGAATTAGATGGTGA
CGTTAACGGTCATAAGTTCTCTGTAAGCGGTGAAGGTGAAGGTGACGCAACATACGGTAAATTAA
CTTTGAAATTCATTTGTACTACAGGTAAATTACCAGTTCCATGGCCAACTTTGGTTACTACTTTG
TCTTGGGGTGTTCAATGTTTCGCTAGATACCCAGATCATATGAAGCAACATGATTTCTTTAAGTC
TGCTATGCCAGAAGGTTACGTTCAAGAAAGAACTATCTTCTTCAAGGATGATGGTAACTATAAGA
CTAGAGCTGAAGTTAAGTTCGAAGGAGATACTTTAGTTAATAGAATTGAATTGAAGGGTATTGAT
TTTAAGGAAGATGGTAATATTTTGGGTCATAAGTTGGAATATAATTACTTCTCTGATAACGTTTA
CATTACTGCTGATAAGCAAAAGAACGGTATTAAGGCGAACTTCAAGATTAGACATAACATTGAAG
ATGGTGGTGTTCAATTAGCTGATCATTACCAACAAAACACTCCAATTGGTGACGGTCCAGTTTTG
TTGCCAGATAACCATTACTTGTCTACTCAATCTAAGTTGTCTAAGGATCCAAACGAAAAGAGAGA
TCATATGGTTTTGTTGGAATTCGTTACTGCTGCTGGGATCACTTTAGGTATGGATGAATTGTATA
AATAG
sfGFP:
ATGTCCAAGGGTGAAGAGCTATTTACTGGGGTTGTACCCATTTTGGTAGAACTGGACGGAGATGT
AAACGGACATAAATTCTCTGTTAGAGGTGAGGGCGAAGGCGATGCCACCAATGGTAAATTGACTC
TGAAGTTTATATGCACTACGGGTAAATTACCTGTTCCTTGGCCAACCCTAGTAACAACTTTGACA
TATGGTGTTCAATGTTTCTCAAGATACCCAGACCATATGAAAAGGCATGATTTCTTTAAAAGTGC
TATGCCAGAAGGCTACGTGCAAGAGAGAACTATCTCCTTTAAGGATGACGGTACGTATAAAACAC
GAGCAGAAGTGAAATTCGAAGGGGATACACTAGTTAATCGCATCGAATTAAAGGGTATAGACTTT
AAGGAAGATGGTAATATTCTCGGCCATAAACTTGAGTATAATTTCAACTCGCATAATGTGTACAT
TACAGCTGACAAACAAAAGAACGGAATTAAAGCGAATTTTAAAATCAGGCACAACGTCGAAGATG
GGTCTGTTCAACTTGCCGATCATTATCAGCAAAACACCCCTATTGGTGATGGTCCAGTCTTGTTA
CCCGATAATCACTACTTAAGCACACAGTCTAGATTGTCAAAAGATCCGAATGAAAAGCGTGATCA
CATGGTTTTATTGGAATTTGTCACCGCTGCAGGAATAACTCACGGAATGGACGAGCTTTATAAGG
GTAG
ytdTomato:
ATGGTTAGTAAAGGTGAGGAAGTGATTAAGGAATTCATGAGGTTTAAAGTTAGAATGGAAGGTTC
TATGAATGGTCATGAATTTGAAATTGAAGGTGAAGGTGAAGGTAGACCATATGAAGGTACACAAA
CTGCTAAATTGAAAGTTACAAAAGGTGGTCCATTACCATTTGCATGGGATATTTTATCTCCACAG
TTTATGTATGGTAGTAAAGCATATGTTAAACATCCAGCTGATATTCCAGATTATAAGAAATTAAG
TTTTCCTGAAGGTTTTAAATGGGAAAGAGTAATGAATTTTGAAGATGGTGGTTTAGTAACAGTTA
CTCAAGATTCTTCTTTACAAGATGGTACATTAATATATAAAGTTAAAATGAGAGGTACTAATTTT
CCACCTGATGGTCCAGTTATGCAGAAGAAAACAATGGGTTGGGAAGCCTCAACTGAAAGATTGTA
CCCTAGAGATGGAGTTTTGAAAGGTGAAATTCATCAAGCTCTTAAGTTAAAAGATGGAGGTCACT
ATTTGGTTGAGTTTAAAACTATATACATGGCTAAAAAACCTGTTCAATTGCCAGGTTATTATTAT
GTTGATACTAAATTAGATATTACTTCACATAATGAAGATTATACTATTGTTGAACAATATGAAAG
ATCTGAAGGTAGACATCATTTGTTTTTGGGTCATGGTACTGGTAGTACTGGTTCTGGTTCTTCAG
GTACTGCTTCTAGTGAAGATAATAATATGGCTGTAATCAAAGAGTTCATGAGGTTTAAAGTTAGA
ATGGAAGGTTCTATGAATGGTCATGAGTTTGAAATCGAAGGTGAAGGAGAAGGTAGACCATATGA
AGGTACTCAAACTGCTAAATTGAAAGTTACTAAAGGTGGTCCATTGCCATTTGCTTGGGATATAT
TGTCACCTCAGTTTATGTATGGTTCTAAAGCATATGTTAAACATCCAGCTGATATTCCTGATTAT
AAAAAATTATCATTTCCTGAAGGTTTTAAATGGGAAAGAGTTATGAATTTTGAAGATGGTGGTTT
AGTTACTGTAACTCAAGATTCTTCATTGCAAGATGGTACATTAATATATAAAGTTAAAATGAGAG
GTACTAATTTTCCACCTGATGGTCCAGTTATGCAGAAAAAGACTATGGGTTGGGAAGCATCTACT
GAAAGATTATATCCTAGAGATGGTGTTTTAAAAGGTGAAATTCACCAAGCCTTAAAATTGAAAGA
TGGTGGTCATTATTTGGTTGAATTCAAAACAATCTACATGGCTAAAAAACCAGTTCAGCTGCCCG
GATACTATTATGTCGATACTAAACTGGATATTACTTCACACAATGAAGACTACACAATTGTTGAA
CAGTATGAAAGATCAGAGGGTAGACATCACTTGTTTTTATACGGTATGGATGAATTATATAAATA
G

AfuSte2 Activation Assay with synthetic peptides and B5233 culture supernatants: AfuSte2 GPCR activity and response to increasing concentrations of synthetic peptide ligands was measure in strain yMJ194 using mCherry as a fluorescent reporter. The strain yDR100, yMJ194 containing the AfuSte2 GPCR expression vector, was assayed in 96-well microtiter plates with a working volume of 200 μl, cultured at 30° C. and 800 rpm. Cells were seeded at an OD600 of 0.3 in SC media deficient in tryptophan (selective component). All measurements were performed in triplicate. Culture turbidity (OD600) and mCherry fluorescence (excitation: 588 nm, emission: 620 nm) were measured in 2 h intervals for 8 h using an Infinite 200 Pro plate reader (Tecan). To account for the optical density values exceeding the linear range of the photo detector, all optical density values were corrected with the following formula to yield true optical density values: A_true=(k·A_meas)/(A_sat−A_meas). Where Asat is the photodetector saturation value, Ameas is the measure optical density, and k is the true optimal density (Ostrov et al. A modular yeast biosensor for low-cost point-of-care pathogen detection. Sci. Adv. 3, (2017)).

LC-MS peptide validation of clinical isolate cultures: B5233 culture supernatants for a 5-day and 13-day time point, and 100 nM synthetic peptide standard were analyzed.

AfuSte2 GFCR orthogonality assay with synthetic peptide: AfuSte2 GPCR activation was individually measure in 96-well microtiter plates in triplicate with each of the synthetic peptides (10 μM). Cells were seeded at an OD600 of 0.3 in 96-well microtiter plates with a working volume of 200 μl, cultured at 30° C. and 800 rpm. Culture turbidity (OD600) and mCherry fluorescence (excitation: 588 nm, emission: 620 nm) were measured in 2 h intervals for 12 h using an Infinite 200 Pro plate reader (Tecan). Percent receptor activation was determined by using the OD600-normalized fluorescence value of the maximum activation of the AfuSte2 GPCR as 100% activation and using the value of the water-treated cells to 0% activation (Billerbeck et al. A scalable peptide-GPCR language for engineering multicellular communication. Nat. Commun. 9, (2018)).

A. fumigatus biosensor strain and signal amplification strain engineering with lycopene readout: Strain yTC412 was constructed with CRISPR/Cas9 genome integration of pGPD_FAD1 and inducible CrtI into ARS208a locus, and integration of pTEF1_CrtE, pGPD_CrtB and inducible CrtI into HO locus. In yTC412, additional biosensing components were integrated into LEU2 locus. Strain yTC527 was constructed with CRISPR/Cas9 genome integration of constitutive AfuSte2 into LEU2 locus of yTC412. Strain yTC528 was constructed with CRISPR/Cas9 genome integration of constitutive AfuSte2 and inducible CrtI into LEU2 locus of yTC412.

Quantification and statistical analysis: Statistical tests of all experiments were performed using GraphPad Prism version 7 or Python version xx and are detailed within the legend of each figure. In all figures, the data points represent mean±SD. Curves fitted to all dose-response data was fitted in Prism 7 using the Nonlinear Regression: Variable slope (four parameter) curve fitting.

### Results

The present example provides an improved yeast biosensor. Previously developed biosensors had relatively high background response and low signal-to-noise ratio, which was particularly evident with the red pigment as a readout, primarily because of the usage of the natural yeast intracellular pheromone signaling pathway and native inducible pFus1 and pFig1 promoters (Neves, S. R., Ram, P. T. & Iyengar, R. G protein pathways. Science (80.). 296, 1636-1639 (2002)).

In this example, an insulated and minimal pheromone signaling pathway, which has refactored expression of the majority of pheromone signaling components in order to minimize basally activated transcription and significantly enhance activated output signal, was used (Shaw, W. M. et al. Engineering a Model Cell for Rational Tuning of GPCR Signaling. Cell 177, 1-15 (2019)). The parent strain (yTC370) was further modified by genome integration of key components of an A. fumigatus yeast biosensor (GPCR expression, peptide secretion and inducible readout expression). In particular, an A. fumigatus GprA mating receptor (AfuSte2), which was codon-optimized for yeast expression, was expressed on a plasmid and inducible mCherry fluorescent protein expression was integrated (yTC479).

The inducibility of AfuSte2 was validated by its activation with the synthetic A. fumigatus pheromone peptide (AfuPep). Like S. cerevisiae, the A. fumigatus repeats of mature peptide sequence are surrounded by cleavage signals which are recognized by conserved proteins involved in pheromone processing, such as KEX1, KEX2 and STE13. The pheromone peptide sequence was reported before and it is predicted to be two repeats of nonapeptide WCHLPGQGC (SEQ ID NO: 3) (Pöggeler, S. & Wöstemeyer, J. Evolution of fungi and fungal-like organisms. (Springer Science & Business Media, 2011)). It was found that AfuSte2 was relatively sensitive to its cognate peptide with an EC₅₀ value (concentration of peptide required for half-maximal activation) of ˜100 nM and was very strongly activated with around 4,000-fold change of fluorescence readout compared to background (FIG. 1B). To probe receptor's specificity to other fungal mating peptides, cross-reactivity was tested with synthetic peptides from species Zygosaccharomyces rouxii (Zr), Zygosaccharomyces bailii (Zb), Schizosaccharomyces japonicus (Sj) Saccharomyces castellii (Scas), Hypocrea jecorina (Hj), Geotrichum candidum (Gc), Candida tenuis (Cn) and Vanderwaltozyma polyspora (Vp) (FIG. 2). AfuSte2 did not get activated by any of the tested peptides, nor did AfuPep activate fungal cognate receptors. Furthermore, to probe receptor's specificity to other pheromone peptides of closely related Aspergillus species, AfuSte2 activation is tested with fungal mating peptides from closely related Aspergillus species, as shown in Example 4.

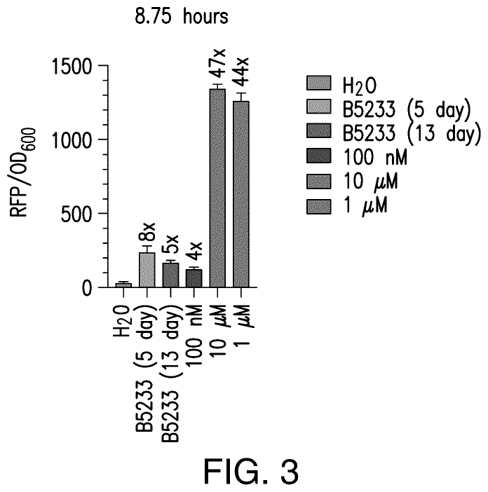

Initial biosensor's induction (yDR01) was tested with A. fumigatus clinical isolate B5233 sterile culture supernatants which were grown for either 5 days or 13 days). The strain showed 8-fold and 5-fold induction after 9-h incubation with the supernatants, respectively (FIG. 3).

### REFERENCES

1. Bongomin, F., Gago, S., Oladele, R. & Denning, D. Global and Multi-National Prevalence of Fungal Diseases—Estimate Precision. J. Fungi 3, 57 (2017).
2. Brown, G. D. et al. Hidden killers: Human fungal infections. Sci. Transl. Med. 4, 1-10 (2012).
3. Fausto, A., Rodrigues, M. L. & Coelho, C. The still underestimated problem of fungal diseases worldwide. Front. Microbiol. 10, 1-5 (2019).
4. Song, G., Liang, G. & Liu, W. Fungal Co-infections Associated with Global COVID-19 Pandemic: A Clinical and Diagnostic Perspective from China. Mycopathologia 185, 599-606 (2020).
5. Van De Veerdonk, F. L., Gresnigt, M. S., Romani, L., Netea, M. G. & Latgé, J. P. Aspergillus fumigatus morphology and dynamic host interactions. Nat. Rev. Microbiol. 15, 661-674 (2017).
6. Latge, J.-P. & Chamilos, G. Aspergillus fumigatus and Aspergillosis in 2019. Clin. Microbiol. Rev. 33, e00140-18 (2020).
7. Comely, O. A., Lass-FlorI, C., Lagrou, K., Arsic-Arsenijevic, V. & Hoenigl, M. Improving outcome of fungal diseases-guiding experts and patients towards excellence. Mycoses 60, (Blackwell Publishing Ltd, 2017).
8. Egger, M., Jenks, J. D., Hoenigl, M. & Prattes, J. Blood Aspergillus PCR: The Good, the Bad, and the Ugly. J. Fungi 6, 1-14 (2020).
9. Jenks, J. D. & Hoenigl, M. Point-of-care diagnostics for invasive aspergillosis: nearing the finish line. Expert Rev. Mol. Diagn. 20, 1009-1017 (2020).
10. Jenks, J. D., Miceli, M. H., Prattes, J., Mercier, T. & Hoenigl, M. The Aspergillus Lateral Flow Assay for the Diagnosis of Invasive Aspergillosis: an Update. Curr. Fungal Infect. Rep. 14, 378-383 (2020).
11. Ostrov, N. et al. A modular yeast biosensor for low-cost point-of-care pathogen detection. Sci. Adv. 3, (2017).
12. Paoletti, M. et al. the mating. Curr. Biol. 15, 1242-1248 (2005).
13. O'Gorman, C. M., Fuller, H. T. & Dyer, P. S. Discovery of a sexual cycle in the opportunistic fungal pathogen Aspergillus fumigatus. Nature 457, 471-474 (2009).
14. Lafon, A., Han, K. H., Seo, J. A., Yu, J. H. & d'Enfert, C. G-protein and cAMP-mediated signaling in aspergilli: A genomic perspective. Fungal Genet. Biol. 43, 490-502 (2006).
15. Neves, S. R., Ram, P. T. & Iyengar, R. G protein pathways. Science (80.). 296, 1636-1639 (2002).
16. Shaw, W. M. et al. Engineering a Model Cell for Rational Tuning of GPCR Signaling. Cell 177, 1-15 (2019).
17. Pöggeler, S. & Wöstemeyer, J. Evolution of fungi and fungal-like organisms. (Springer Science & Business Media, 2011).
18. Wickes, B. L. & Wiederhold, N. P. Molecular diagnostics in medical mycology. Nat. Commun. 9, 1-13 (2018).
19. Billerbeck, S. et al. A scalable peptide-GPCR language for engineering multicellular communication. Nat. Commun. 9, (2018).
20. Shimada, H. et al. Increased carotenoid production by the food yeast Candida utilis through metabolic engineering of the isoprenoid pathway. Appl. Environ. Microbiol. 64, 2676-2680 (1998).
21. Daniel Gietz, R. & Schiestl, R. H. High-efficiency yeast transformation using the LiAc/SS carrier DNA/PEG method. Nat. Protoc. 2, 31-34 (2007).

### Example 2: Building Modular Biosensing Component Golden Gate Assembly Toolkit

In order to start with the improvement of the A. fumigatus biosensor, a modular and straightforward way to easily interchange relevant biosensing components and integrate them in a common parent yeast strain was developed. First, which biosensing components were going to be part of the parent yeast strain and which were going to be part of modular mix-and-match integration system for that parent strain were identified. The biosensing mechanism in the live yeast biosensors is composed of three essential components: the GPCR, the intracellular signaling pathway which gets activated upon cognate peptide binding to GPCR, and the target readout gene whose expression is driven under peptide/GPCR-inducible promoters. The common component to all the biosensors is the intracellular signaling pathway. The components which are interchanged depending on the target peptide to be detected and what type of readout wanted are the GPCRs and genes under inducible promoters. Therefore, a parent strain which would lack the GPCR and inducible readouts while retaining all other intracellular signaling components was engineered.

Materials and Methods
Constitutive GPCR cassette:
pTDH3-GPCR-tSTE2
GGTCTCACAGTAGTTTATCATTATCAATACTGCCATTTCAAAGAATACGTAAATAATTAATAGTA
GTGATTTTCCTAACTTTATTTAGTCAAAAAATTAGCCTTTTAATTCTGCTGTAACCCGTACATGC
CCAAAATAGGGGGCGGGTTACACAGAATATATAACATCGTAGGTGTCTGGGTGAACAGTTTATTC
CTGGCATCCACTAAATATAATGGAGCCCGCTTTTTAAGCTGGCATCCAGAAAAAAAAAGAATCCC
AGCACCAAAATATTGTTTTCTTCACCAACCATCAGTTCATAGGTCCATTCTCTTAGCGCAACTAC
AGAGAACAGGGGCACAAACAGGCAAAAAACGGGCACAACCTCAATGGAGTGATGCAACCTGCCTG
GAGTAAATGATGACACAAGGCAATTGACCCACGCATGTATCTATCTCATTTTCTTACACCTTCTA
TTACCTTCTGCTCTCTCTGATTTGGAAAAAGCTGAAAAAAAAGGTTGAAACCAGTTCCCTGAAAT
TATTCCCCTACTTGACTAATAAGTATATAAAGACGGTAGGTATTGATTGTAATTCTGTAAATCTA
TTTCTTAAACTTCTTAAATTCTACTTTTATAGTTAGTCTTTTTTTTAGTTTTAAAACACCAAGAA
CTTAGTTTCGACGGATACTAGTAAA[receptorORF]CTCGAGACGGCTTTGAAAAAGTAATTT
CGTGACCTTCGGTATAAGGTTACTACTAGATTCAGGTGCTCATCAGATGCACCACATTCTCTATA
AAAAAAAATGGTATCTTTCTTATTTGATAATATTTAAACTCCTTTACATAATAAACATCTCGTAA
GTAGTGGTAGAAACCACCTTTGCTTTTACGAGTTCACCAACGAGACC
Inducible readout cassette:
LexO(6x)-pLEU2m-readout-tTDH1
GGTCTCACCAAAAACGGGTAGTCCATCGTTGTAGGATACTGTATATACACCCAGTAGAGTAGGTG
ACTACTGTATGAGCATACAGTAGTGACAACCACTTACTGTATATAAATACAGTAGTGGTCATCGA
TACTGTATATAAAACCAGTAGGCAATCGTTTGTACTGTATGTACATACAGTATACCTCGCAACTA
CTGTATATAAACACAGTAATTAACTTGTAATATTCTAATCAATTGACAATATTATTTAAGGACCT
ATTGTTTTTTCCAATAGGTGGTTAGCAATCGTCTTACTTTCTAACTTTTCTTACCTTTTACATTT
CAGCAATATATATATATATATTTCAAGGATATACCATTCTAAGATCTG[readoutORF]CTAAC
TCGAGATAAAGCAATCTTGATGAGGATAATGATTTTTTTTTGAATATACATAAATACTACCGTTT
TTCTGCTAGATTTTGTGATGACGTAAATAAGTACATATTACTTTTTAAGCCAAGACAAGATTAAG
CATTAACTTTACCCTTTTCTTTCTAAGTTTCAATATTAGTTATCACTGTTTAAAAGTTATGGCGA
GAACGTCGGCGGTTAAAATATATTACCCTGAACGGCATCGAGACC
LexO(6x)-pLEU2m-CrtI-tADH1(tHMG1-pCCW12, rev)
GGTCTCACCAAAAACGGGTAGTCCATCGTTGTAGGATACTGTATATACACCCAGTAGAGTAGGTG
ACTACTGTATGAGCATACAGTAGTGACAACCACTTACTGTATATAAATACAGTAGTGGTCATCGA
TACTGTATATAAAACCAGTAGGCAATCGTTTGTACTGTATGTACATACAGTATACCTCGCAACTA
CTGTATATAAACACAGTAATTAACTTGTAATATTCTAATCAATTGACAATATTATTTAAGGACCT
ATTGTTTTTTCCAATAGGTGGTTAGCAATCGTCTTACTTTCTAACTTTTCTTACCTTTTACATTT
CAGCAATATATATATATATATTTCAAGGATATACCATTCTAAGATCTatgaagaaaaccgtagtg
attggtgcaggttttggtggtttagctttggctatacgtctacaagctgcaggtattcctacagt
gctattggagcaaagagacaaaccaggaggaagagcttatgtttggcacgatcaaggctttacct
ttgatgctggtcctacagtcatcactgatcctactgcattggaagctttgttcaccttagctggt
agaagaatggaagattatgtccgtctattgcctgtcaagccgttttacagattgtgttgggaatc
tggtaaaaccctagattacgccaatgacagtgctgaactagaagctcagattacgcagtttaatc
ccagagatgtcgaaggttacaggagattccttgcctattcccaagctgttttccaagagggttat
cttcgtttgggttcagttccattcctgtcctttagggatatgcttagagcaggtcctcagttgtt
gaagctacaagcatggcaaagtgtgtatcagtctgtttcgagatttatcgaggatgaacatctga
gacaagcattctcattccacagtcttctagttggaggtaatccctttaccacatcgagcatatat
acgttgattcacgctttggaaagagaatggggagtttggtttcctgaaggtggaacaggtgcttt
ggttaatggtatggtgaagctattcacggatttgggtggagaaatagagctgaatgcaagagtgg
aagaacttgttgtagcagacaacagagtctcacaagttagacttgctgatggtaggatcttcgat
acagatgctgtagcttcaaacgcagatgtagtgaacacttataaaaagttgttgggacatcatcc
tgttggacaaaagagagcagctgctttggagaggaaatctatgagcaactcgttgtttgtccttt
actttgggctgaatcaaccacactcacaactagctcatcacacaatctgctttggtcctagatac
agagagctgatagatgaaattttcactggatctgctttagcagacgatttttccctgtacttgca
ttcaccatgtgttactgatccctctttagcaccacctggttgtgctagcttctatgtactagcac
ctgtaccacatttgggtaatgctccattagattgggcacaagaaggaccgaaattgagggatagg
atcttcgactatttggaagaacgttacatgccaggtttgagatctcagttggttacacagaggat
attcacaccagctgattttcatgatactctagatgcgcatttaggtagcgctttttccattgagc
cacttttgacgcaaagtgcttggtttagaccacacaacagagattctgacattgccaatctgtac
ctagtaggtgcaggaactcatccaggagctggtattcctggagttgtagcttctgctaaagctac
tgctagtctgatgatcgaggatttgcagtaaGCGAATTTCTTATGATTTATGATTTTTATTATTA
AATAAGTTATAAAAAAAATAAGTGTATACAAATTTTAAAGTGACTCTTAGGTTTTAAAACGAAAA
TTCTTATTCTTGAGTAACTCTTTCCTGTAGGTCAGGTTGCTTTCTCAGGTATAGCATGAGGTCGC
TCttaggatttaatgcaggtgacggacccatctttcaaacgatttatatcagtggcgtccaaatt
gttaggttttgttggttcagcaggtttcctgttgtgggtcatatgactttgaaccaaatggccgg
ctgctagggcagcacataaggataattcacctgccaagacggcacaggcaactattcttgctaat
tgacgtgcgttggtaccaggagcggtagcatgcgggcctcttacacctaataagtccaacatggc
accttgtggttctagaacagtaccaccaccgatggtacctacttcgatggatggcatggatacgg
aaattctcaaatcaccgtccacttctttcatcaatgttatacagttggaactttcaacattttgt
gcaggatcttgtcctaatgccaagaaaacagctgtcactaaattagctgcatgtgcgttaaatcc
accaacagacccagccattgcagatccaaccaaattcttagcaatgttcaactcaaccaatgcgg
aaacatcactttttaacacttttctgacaacatcaccaggaatagtagcttctgcgacgacactc
ttaccacgaccttcgatccagttgatggcagctggttttttgtcggtacagtagttaccagaaac
ggagacaacctccatatcttcccagccatactcttctaccatttgctttaatgagtattcgacac
ctttagaaatcatattcatacccattgcgtcaccagtagttgttctaaatctcatgaagagtaaa
tctcctgctagacaagtttgaatatgttgcagacgtgcaaatcttgatgtagagttaaaagcttt
tttaattgcgttttgtccctcttctgagtctaaccatatcttacaggcaccagatcttttcaaag
ttgggaaacggactactgggcctcttgtcataccatccttagttaaaacagttgttgcaccaccg
ccagcattgattgccttacagccacgcatggcagaagctaccaaacaaccctctgtagttgccat
tggtatatgataagatgtaccatcgataaccaaggggcctataacaccaacgggcaaaggcatgt
aacctataacattttcacaacaagcgccaaatacgcggtcgtagtcataatttttatatggtaaa
cgatcagatgctaatacaggagcttctgccaaaattgaaagagccttcctacgtaccgcaaccgc
tctcgtagtatcacctaattttttctccaaagcgtacaaaggtaacttaccgtgaataaccaagg
cagcgacctctttgttcttcaattgttttgtatttccactacttaataatgcttctaattcttct
aaaggacgtattttcttatccaagctttcaatatcgcgggaatcatcttcctcactagatgatga
aggtcctgatgagctcgattgcgcagatgataaacttttgactttcgatccagaaatgactgttt
tattggttaaaactggtgtagaagccttttgtacaggagcagtaaaagacttcttggtgacttca
gttttcaccaattggtccatGATCTTTATTGATATAGTGTTTAAGCGAATGACAGAAGATTAATT
TCTTGGTATGTTAGGAAAGAATAAAGGAGAATAAGAATAATTAGAACAATGTAGGATGGAAAGAA
AGATTATCAAGCATGCCGACTTTATATACTTGAACGGAGGCAAAGGATGCAAAATTTTCTCACAT
TTCTTTCTGCCGTTATGTTGGAAGTAAGACTCCCATTATCGCAATACTGCAACACGAATATGCAA
AATTTGCTGAGTTATCGCAGATAGTTGTTGCAAAGATAGCGGCGTAGGTGGCCGCGAAATGGGAA
ATTCCAAAACAAACGGTTTTTTACTCCTGAGAAATACTTGTACGGGATAATCCAGGGCCTACCAC
CCACGCTTCGAGGATTGGCTTTTATTTTTTTTTTTTTGGTGGCGTTTTATTTCTTTCCCGCTTTC
TGGGACTTGTGCGGAGTTTTGAGAGGGGCGCGCGGCAAAGGATTCCCAAAACGGAAATCAGACGC
CAATAGCCAGCACTCAAAGCAGTTCTGGACCCATTCCGATTTTCCCATTTGGTTCTTGCGCGTGC
TGATTCCGACACGCGCGTCTATAAATAGCATGAAGTATCCGCACACCGCAGCGTTAGTGAGGTGA
GGGTGGCAGCAAGCTAATTCCCGCATCTGGAATCTGAACTGCCCCTTTTGGACTAACCGTGTGGT
TCATGGGTGGCATCGAGACC
LexO(6x)-pLEU2m-CrtI-tADH1(tHMG1-pCCW12, rev)
pHHF2-ERG20-tPGKI
GGTCTCACCAAAAACGGGTAGTCCATCGTTGTAGGATACTGTATATACACCCAGTAGAGTAGGTG
ACTACTGTATGAGCATACAGTAGTGACAACCACTTACTGTATATAAATACAGTAGTGGTCATCGA
TACTGTATATAAAACCAGTAGGCAATCGTTTGTACTGTATGTACATACAGTATACCTCGCAACTA
CTGTATATAAACACAGTAATTAACTTGTAATATTCTAATCAATTGACAATATTATTTAAGGACCT
ATTGTTTTTTCCAATAGGTGGTTAGCAATCGTCTTACTTTCTAACTTTTCTTACCTTTTACATTT
CAGCAATATATATATATATATTTCAAGGATATACCATTCTAAGATCTatgaagaaaaccgtagtg
attggtgcaggttttggtggtttagctttggctatacgtctacaagctgcaggtattcctacagt
gctattggagcaaagagacaaaccaggaggaagagcttatgtttggcacgatcaaggctttacct
ttgatgctggtcctacagtcatcactgatcctactgcattggaagctttgttcaccttagctggt
agaagaatggaagattatgtccgtctattgcctgtcaagccgttttacagattgtgttgggaatc
tggtaaaaccctagattacgccaatgacagtgctgaactagaagctcagattacgcagtttaatc
ccagagatgtcgaaggttacaggagattccttgcctattcccaagctgttttccaagagggttat
cttcgtttgggttcagttccattcctgtcctttagggatatgcttagagcaggtcctcagttgtt
gaagctacaagcatggcaaagtgtgtatcagtctgtttcgagatttatcgaggatgaacatctga
gacaagcattctcattccacagtcttctagttggaggtaatccctttaccacatcgagcatatat
acgttgattcacgctttggaaagagaatggggagtttggtttcctgaaggtggaacaggtgcttt
ggttaatggtatggtgaagctattcacggatttgggtggagaaatagagctgaatgcaagagtgg
aagaacttgttgtagcagacaacagagtctcacaagttagacttgctgatggtaggatcttcgat
acagatgctgtagcttcaaacgcagatgtagtgaacacttataaaaagttgttgggacatcatcc
tgttggacaaaagagagcagctgctttggagaggaaatctatgagcaactcgttgtttgtccttt
actttgggctgaatcaaccacactcacaactagctcatcacacaatctgctttggtcctagatac
agagagctgatagatgaaattttcactggatctgctttagcagacgatttttccctgtacttgca
ttcaccatgtgttactgatccctctttagcaccacctggttgtgctagcttctatgtactagcac
ctgtaccacatttgggtaatgctccattagattgggcacaagaaggaccgaaattgagggatagg
atcttcgactatttggaagaacgttacatgccaggtttgagatctcagttggttacacagaggat
attcacaccagctgattttcatgatactctagatgcgcatttaggtagcgctttttccattgagc
cacttttgacgcaaagtgcttggtttagaccacacaacagagattctgacattgccaatctgtac
ctagtaggtgcaggaactcatccaggagctggtattcctggagttgtagcttctgctaaagctac
tgctagtctgatgatcgaggatttgcagtaaGCGAATTTCTTATGATTTATGATTTTTATTATTA
AATAAGTTATAAAAAAAATAAGTGTATACAAATTTTAAAGTGACTCTTAGGTTTTAAAACGAAAA
TTCTTATTCTTGAGTAACTCTTTCCTGTAGGTCAGGTTGCTTTCTCAGGTATAGCATGAGGTCGC
TCttaggatttaatgcaggtgacggacccatctttcaaacgatttatatcagtggcgtccaaatt
gttaggttttgttggttcagcaggtttcctgttgtgggtcatatgactttgaaccaaatggccgg
ctgctagggcagcacataaggataattcacctgccaagacggcacaggcaactattcttgctaat
tgacgtgcgttggtaccaggagcggtagcatgcgggcctcttacacctaataagtccaacatggc
accttgtggttctagaacagtaccaccaccgatggtacctacttcgatggatggcatggatacgg
aaattctcaaatcaccgtccacttctttcatcaatgttatacagttggaactttcaacattttgt
gcaggatcttgtcctaatgccaagaaaacagctgtcactaaattagctgcatgtgcgttaaatcc
accaacagacccagccattgcagatccaaccaaattcttagcaatgttcaactcaaccaatgcgg
aaacatcactttttaacacttttctgacaacatcaccaggaatagtagcttctgcgacgacactc
ttaccacgaccttcgatccagttgatggcagctggttttttgtcggtacagtagttaccagaaac
ggagacaacctccatatcttcccagccatactcttctaccatttgctttaatgagtattcgacac
ctttagaaatcatattcatacccattgcgtcaccagtagttgttctaaatctcatgaagagtaaa
tctcctgctagacaagtttgaatatgttgcagacgtgcaaatcttgatgtagagttaaaagcttt
tttaattgcgttttgtccctcttctgagtctaaccatatcttacaggcaccagatcttttcaaag
ttgggaaacggactactgggcctcttgtcataccatccttagttaaaacagttgttgcaccaccg
ccagcattgattgccttacagccacgcatggcagaagctaccaaacaaccctctgtagttgccat
tggtatatgataagatgtaccatcgataaccaaggggcctataacaccaacgggcaaaggcatgt
aacctataacattttcacaacaagcgccaaatacgcggtcgtagtcataatttttatatggtaaa
cgatcagatgctaatacaggagcttctgccaaaattgaaagagccttcctacgtaccgcaaccgc
tctcgtagtatcacctaattttttctccaaagcgtacaaaggtaacttaccgtgaataaccaagg
cagcgacctctttgttcttcaattgttttgtatttccactacttaataatgcttctaattcttct
aaaggacgtattttcttatccaagctttcaatatcgcgggaatcatcttcctcactagatgatga
aggtcctgatgagctcgattgcgcagatgataaacttttgactttcgatccagaaatgactgttt
tattggttaaaactggtgtagaagccttttgtacaggagcagtaaaagacttcttggtgacttca
gttttcaccaattggtccatGATCTTTATTGATATAGTGTTTAAGCGAATGACAGAAGATTAATT
TCTTGGTATGTTAGGAAAGAATAAAGGAGAATAAGAATAATTAGAACAATGTAGGATGGAAAGAA
AGATTATCAAGCATGCCGACTTTATATACTTGAACGGAGGCAAAGGATGCAAAATTTTCTCACAT
TTCTTTCTGCCGTTATGTTGGAAGTAAGACTCCCATTATCGCAATACTGCAACACGAATATGCAA
AATTTGCTGAGTTATCGCAGATAGTTGTTGCAAAGATAGCGGCGTAGGTGGCCGCGAAATGGGAA
ATTCCAAAACAAACGGTTTTTTTACTCCTGAGAAATACTTGTACGGGATAATCCAGGGCCTACCA
CCCACGCTTCGAGGATTGGCTTTTATTTTTTTTTTTTTGGTGGCGTTTTATTTCTTTCCCGCTTT
CTGGGACTTGTGCGGAGTTTTGAGAGGGGCGCGCGGCAAAGGATTCCCAAAACGGAAATCAGACG
CCAATAGCCAGCACTCAAAGCAGTTCTGGACCCATTCCGATTTTCCCATTTGGTTCTTGCGCGTG
CTGATTCCGACACGCGCGTCTATAAATAGCATGAAGTATCCGCACACCGCAGCGTTAGTGAGGTG
AGGGTGGCAGCAAGCTAATTCCCGCATCTGGAATCTGAACTGCCCCTTTTGGACTAACCGTGTGG
TTCATGGGTGCCCCTGTGGAGTGTTTGCTTGGATCCTTTAGTAAAAGGGGAAGAACAGTTGGAAG
GGCCAAAGTGGAAGTCACAAAACAGTGGTCCTATATAAAAGAACAAGAAAAAGATTATTTATATA
CAACTGCGGTCACAAGAAGCAACGCGAGAGAGCACAACACGCTGTTATCACGCAAACTATGTTTT
GACACCGAGCCATAGCCGTGATTGTGCGTCACATTGGGCGATAATGAACGCTAAATGACCAACTC
CCATCCGTAGGAGCCCCTTAGGGCGTGCCAATAGTTTCACGCGCTTAATGCGAAGTGCTCGGAAC
GGACAACTGTGGTCGTTTGGCACCGGGAAAGTGGTACTAGACCGAGAGTTTCGCATTTGTATGGC
AGGACGTTCTGGGAGCTTCGCGTCTAAAGCTTTTTCGGGCGCGAAATGCAGACCAGACCAGAACA
AAACAACTGACAAGAAGGCGTTTAATTTAATATGTTGTTCACTCGCGCCTGGGCTGTTGTTATTC
GGCTAGATACATACGTGTTTGTGCGTATGTAGTTATATCATATATAAGTATATTAGGATGAGGCG
GTGAAAGAGATTTTTTTTTTTTCGCTTAATTTATTCTTTTCTCTATCTTTTTTCCTACATCTTGT
TCAAAAGAGTAGCAAAAACAACAATCAATACAATAAAATAatggcttcagaaaaagaaattagga
gagagagattcttgaacgttttccctaaattagtagaggaattgaacgcatcgcttttggcttac
ggtatgcctaaggaagcatgtgactggtatgcccactcattgaactacaacactccaggcggtaa
gttaaatagaggtttgtccgttgtggacacgtatgctattctctccaacaagaccgttgaacaat
tggggcaagaagaatacgaaaaggttgctattctaggttggtgcattgagttgttgcaggcttac
ttcttggtcgccgatgatatgatggacaagtccattaccagaagaggccaaccatgttggtacaa
ggttcctgaagttggggaaattgccatcaatgacgcattcatgttagaggctgctatctacaagc
ttttgaaatctcacttcagaaacgaaaaatactacatagatatcaccgaattgttccatgaagtc
accttccaaaccgaattgggccaattgatggacttaatcactgcacctgaagacaaagtcgactt
gagtaagttctccctaaagaagcactccttcatagttactttcaagactgcttactattctttct
acttgcctgtcgcattggctatgtacgttgccggtatcacagatgaaaaggatttgaaacaagcc
agagatgtcttgattccattgggtgaatatttccaaattcaagatgactacttagactgcttcgg
taccccagaacagatcggtaagatcggtacagatatccaagataacaaatgttcttgggtaatca
acaaggcattagaacttgcttccgcagaacaaagaaagactttagacgaaaattacggtaagaag
gactcagtcgcagaagccaaatgcaaaaagattttcaatgacttgaaaatcgaccagttatacca
cgaatatgaagagtctgttgccaaggatttgaaggccaagatctcccaagtcgacgagtctcgtg
gcttcaaagccgacgtcttaactgcgtttttgaacaaggtttacaagagaagtaaataaCTCGAG
ATTGAATTGAATTGAAATCGATAGATCAATTTTTTTCTTTTCTCTTTCCCCATCCTTTACGCTAA
AATAATAGTTTATTTTATTTTTTGAATATTTTTTATTTATATACGTATATATAGACTATTATTTA
TCTTTTAATGATTATTAAGATTTTTATTAAAAAAAAATTCGCTCCTCTTTTAATGCCTTTATGCA
GTTTTTTTTTCCCATTCGATATTTCTATGTGCATCGAGACC
Inducible peptide secretion cassette:
LexO(4x)-pADL6m-pre-pro peptide-tCYC1
GGTCTCAGCATGGTAGTCCATCGTTGTAGGATACTGTATATAAATACAGTAGTGGTCATCGATAC
TGTATATAAAACCAGTAGGCAATCGTTTGTACTGTATGTACATACAGTATACCTCGCAACTACTG
TATATAAACACAGTAATTAACTTGTAATATTCTAATCAATTGATATATAAATGTAATAAGAAGTT
TGGTAATATTCAATTCGAAGTGTTCAGTCTTTTACTTCTCTTGTTTTATAGAAGAAAAAACATCA
AGAAACATCTTTAACATACACAAACACATACTATCAGAATACAAGATCTTACA[prepropepti
deORF]GACAGGCCCCTTTTCCTTTGTCGATATCATGTAATTAGTTATGTCACGCTTACATTCAC
GCCCTCCTCCCACATCCGCTCTAACCGAAAAGGAAGGAGTTAGACAACCTGAAGTCTAGGTCCCT
ATTTATTTTTTTTAATAGTTATGTTAGTATTAAGAACGTTATTTATATTTCAAATTTTTCTTTTT
TTTCTGTACAAACGCGTGTACGCATGTAACATTATACTGAAAACCTTGCTTGAGAAGGTTTTGGG
ACGCTCGAAGGCTTTAATTTGCGAGTCGAGACC
Strong constitutive peptide protease cassette:
pCCW12-protease-tENO2
GGTCTCAGAGTCACCCATGAACCACACGGTTAGTCCAAAAGGGGCAGTTCAGATTCCAGATGCGG
GAATTAGCTTGCTGCCACCCTCACCTCACTAACGCTGCGGTGTGCGGATACTTCATGCTATTTAT
AGACGCGCGTGTCGGAATCAGCACGCGCAAGAACCAAATGGGAAAATCGGAATGGGTCCAGAACT
GCTTTGAGTGCTGGCTATTGGCGTCTGATTTCCGTTTTGGGAATCCTTTGCCGCGCGCCCCTCTC
AAAACTCCGCACAAGTCCCAGAAAGCGGGAAAGAAATAAAACGCCACCAAAAAAAAAAAAATAAA
AGCCAATCCTCGAAGCGTGGGTGGTAGGCCCTGGATTATCCCGTACAAGTATTTCTCAGGAGTAA
AAAAACCGTTTGTTTTGGAATTTCCCATTTCGCGGCCACCTACGCCGCTATCTTTGCAACAACTA
TCTGCGATAACTCAGCAAATTTTGCATATTCGTGTTGCAGTATTGCGATAATGGGAGTCTTACTT
CCAACATAACGGCAGAAAGAAATGTGAGAAAATTTTGCATCCTTTGCCTCCGTTCAAGTATATAA
AGTCGGCATGCTTGATAATCTTTCTTTCCATCCTACATTGTTCTAATTATTCTTATTCTCCTTTA
TTCTTTCCTAACATACCAAGAAATTAATCTTCTGTCATTCGCTTAAACACTATATCAATAAAGAT
CACTAGT[proteaseORF]ACTAGTTAACTCGAGAGTGCTTTTAACTAAGAATTATTAGTCTTT
TCTGCTTATTTTTTCATCATAGTTTAGAACACTTTATATTAACGAATAGTTTATGAATCTATTTA
GGTTTAAAAATTGATACAGTTTTATAAGTTACTTTTTCAAAGACTCGTGCTGTCTATTGCATAAT
GCACTGGAAGGGGAAAAAAAAGGTGCACACGCGTGGCTTTTTCTTGAATTTGCAGTTTGAAAAAT
TTTTCGAGACC
Intermediate constitutive peptide protease cassette:
pRPL18B-protease-tENO2
GGTCTCAGAGTAAGAGGATGTCCAATATTTTTTTTAAGGAATAAGGATACTTCAAGACTAGATTC
CCCCCTGCATTCCCATCAGAACCGTAAACCTTGGCGCTTTCCTTGGGAAGTATTCAAGAAGTGCC
TTGTCCGGTTTCTGTGGCTCACAAACCAGCGCGCCCGATATGGCTTTCTTTTCACTTATGAATGT
ACCAGTACGGGACAATTAGAACGCTCCTGTAACAATCTCTTTGCAAATGTGGGGTTACATTCTAA
CCATGTCACACTGCTGACGAAATTCAAAGTAAAAAAAAATGGGACCACGTCTTGAGAACGATAGA
TTTTCTTTATTTTACATTGAACAGTCGTTGTCTCAGCGCGCTTTATGTTTTCATTCATACTTCAT
ATTATAAAATAACAAAAGAAGAATTTCATATTCACGCCCAAGAAATCAGGCTGCTTTCCAAATGC
AATTGACACTTCATTAGCCATCACACAAAACTCTTTCTTGCTGGAGCTTCTTTTAAAAAAGACCT
CAGTACACCAAACACGTTACCCGACCTCGTTATTTTACGACAACTATGATAAAATTCTGAAGAAA
AAATAAAAAAATTTTCATACTTCTTGCTTTTATTTAAACCATTGAATGATTTCTTTTGAACAAAA
CTACCTGTTTCACCAAAGGAAATAGAAAGAAAAAATCAATTAGAAGAAAACAAAAAACAAAAGAT
CT[proteaseORF]ACTAGTTAACTCGAGAGTGCTTTTAACTAAGAATTATTAGTCTTTTCTGC
TTATTTTTTCATCATAGTTTAGAACACTTTATATTAACGAATAGTTTATGAATCTATTTAGGTTT
AAAAATTGATACAGTTTTATAAGTTACTTTTTCAAAGACTCGTGCTGTCTATTGCATAATGCACT
GGAAGGGGAAAAAAAAGGTGCACACGCGTGGCTTTTTCTTGAATTTGCAGTTTGAAAAATTTTTC
GAGACC
Low constitutive peptide protease cassette:
pRAD27-protease-tENO2
GGTCTCAGAGTCCTTGTGAAATTGCAAATATGGTGATTTGAAACGTTTCCTAGTGCAGCAGGATC
ACAGATAACGTGTAAAGGGCTTAGCAGTTGATAATCCTCTCTAGTTAAGACCTAAACAAAATGCT
GTCACTAACCGTAGTATTAAATGACACACTTTGGTGACTTTCGTTAATGGGGATGTGGTAGTGGC
CATTGCCAATAAACAAAAAGAACAGGGAAAGAAGTAGAAAGTGATATAAGTTTGCTTGCCACTTT
TCGTTTTTCACGAAAAAAACAGGCGAAAAAAAATGCTAGACAAGTACCCGGCTGAATCACACCTC
GTTAACAGTGACTTTCGGTGACAGATACCCGATTGGGCACCCGGCTGGTAAGTTATGATAGAAAG
CCAACGCTGTACTATTGGCTTAGCTATGGCAATATTTTGATTATCAGCTAGTTTTATTAACGTTA
TAATTAGTGTAACCAGTTTTTCATCTATTTCATTTATTTCATTTATTTACTTTAATTGCAGATCC
CCCTAACGCGTTTAAAGCTTTTATTCACTAGCTTATGTATTTTTTATAGGAAACGCGACGCGTAA
CATCGCGCAAATGAAGGTTTTGATGTATTATAATGAGGTATTCTTCCTTATATACATCGATGAAA
AGCGTTGACAGCATACATTGGAAAGAAATAGGAAACGGACACCGGAAGAAAAAATAGATCT[pro
teaseORF]ACTAGTTAACTCGAGAGTGCTTTTAACTAAGAATTATTAGTCTTTTCTGCTTATTT
TTTCATCATAGTTTAGAACACTTTATATTAACGAATAGTTTATGAATCTATTTAGGTTTAAAAAT
TGATACAGTTTTATAAGTTACTTTTTCAAAGACTCGTGCTGTCTATTGCATAATGCACTGGAAGG
GGAAAAAAAAGGTGCACACGCGTGGCTTTTTCTTGAATTTGCAGTTTGAAAAATTTTTCGAGACC
Connector cassettes:
CCAA-GCAT
GGTCTCACCAAGCGCCGGGCGCATCGAGACC
GCAT-GAGT
GGTCTCAGCATGCGCCGGGCGAGTCGAGACC
GAGT-TTTT
GGTCTCAGAGTGCGCCGGGCTTTTCGAGACC
GCAT-TTTT
GGTCTCAGCATGCGCCGGGCTTTTCGAGACC
Lycopene cassettes:
pTEF1-CrtE-tADHI-(CrtB-pPGK1, rev), LexO(6x)-
pLEU2m-CrtI-tACTI
GGTCTCACAGTATAGCTTCAAAATGTTTCTACTCCTTTTTTACTCTTCCAGATTTTCTCGGACTC
CGCGCATCGCCGTACCACTTCAAAACACCCAAGCACAGCATACTAAATTTCCCCTCTTTCTTCCT
CTAGGGTGTCGTTAATTACCCGTACTAAAGGTTTGGAAAAGAAAAAAGAGACCGCCTCGTTTCTT
TTTCTTCGTCGAAAAAGGCAATAAAAATTTTTATCACGTTTCTTTTTCTTGAAAATTTTTTTTTT
TGATTTTTTTCTCTTTCGATGACCTCCCATTGATATTTAAGTTAATAAACGGTCTTCAATTTCTC
AAGTTTCAGTTTCATTTTTCTTGTTCTATTACAACTTTTTTTACTTCTTGCTCATTAGAAAGAAA
GCATAGCAATCTAATCTAAGTTTTAATTACAAAatggtttctggttcgaaagcaggagtatcacc
tcatagggaaatcgaagtcatgagacagtccattgatgaccacttagcaggattgttgccagaaa
cagattcccaggatatcgttagccttgctatgagagaaggtgttatggcacctggtaaacgtatc
agacctttgctgatgttacttgctgcaagGgacctgagatatcagggttctatgcctacactact
ggatctagcttgtgctgttgaactgacacatactgcttccttgatgctggatgacatgccttgta
tggacaatgcggaacttagaagaggtcaaccaacaacccacaagaaattcggagaatctgttgcc
attttggcttctgtaggtctgttgtcgaaagcttttggcttgattgctgcaactggtgatcttcc
aggtgaaaggagagcacaagctgtaaacgagctatctactgcagttggtgttcaaggtctagtct
taggacagttcagagatttgaatgacgcagctttggacagaactcctgatgctatcctgtctacg
aaccatctgaagactggcatcttgttctcagctatgttgcaaatcgtagccattgcttctgcttc
ttcaccatctactagggaaacgttacacgcattcgcattggactttggtcaagcctttcaactgc
tagacgatttgagggatgatcatccagagacaggtaaagaccgtaacaaagacgctggtaaaagc
actctagtcaacagattgggtgctgatgcagctagacagaaactgagagagcacattgactctgc
tgacaaacacctgacatttgcatgtccacaaggaggtgctataaggcagtttatgcacctatggt
ttggacaccatcttgctgattggtcCccagtgatgaagatcgcctaaGCGAATTTCTTATGATTT
ATGATTTTTATTATTAAATAAGTTATAAAAAAAATAAGTGTATACAAATTTTAAAGTGACTCTTA
GGTTTTAAAACGAAAATTCTTATTCTTGAGTAACTCTTTCCTGTAGGTCAGGTTGCTTTCTCAGG
TATAGCATGAGGTCGCTCttagacaggtctttgccataaaccagcaggtcttggtgtaactctgg
ttgttttggcacgaatgacttgtcctggtgcagccataagcatcgcaatcttctctcctttgcta
gtgtgttgacgtctatcccaagcagaaccacctgcagcttttaccttgatgccaatctctctgta
gacagatcttgcagtagctatagcccaagcacatctaggtggtagatcatgcaatccagcttgac
ttgagatgtaatagggttcagcagcgtctatcagcctttcagcaactcttgctaatgcagctctg
ttctctcttgcagcgtaattctcaggagttagaccagcatcttgcaaccattcagcaggtagata
gcatctgtcaatagctgcatcgtcgataatatctctcgcgatgtttgtcagctgaaaagccaaac
ctagatcacaagctctgtccaaaaccctttcgtctcttacacccattactcttgccatcatcaaa
ccaactactccagcaacatggtagcagtatctcaaggtgtcttcaaaggtcacgtaacgagtttg
agcaacatccattgcgaaaccatccaagtgatcaagtgccattcttggcgtaataccgtgtgtta
gtgcaacttcttggaatgcagcaaaagcaggatcttgcatctcagcaccttcaaaagctgcaagt
gtaagcgttctcaatctagccaatctctgagtagcctcttcttctgcagcagcttcagatgcgaa
accatgtgtctggtcatctataacgtcatcacagtgtctacaccaagtgtacagcataagcactg
atcttctagtagctgggtcaaacagtttagctgctgtagcaaaggacttggaaccattagccatc
gtttgagtagcatgatccaacaaaggtggttgactcatTGTTTTATATTTGTTGTAAAAAGTAGA
TAATTACTTCCTTGATGATCTGTAAAAAAGAGAAAAAGAAAGCATCTAAGAACTTGAAAAACTAC
GAATTAGAAAAGACCAAATATGTATTTCTTGCATTGACCAATTTATGCAAGTTTATATATATGTA
AATGTAAGTTTCACGAGGTTCTACTAAACTAAACCACCCCCTTGGTTAGAAGAAAAGAGTGTGTG
AGAACAGGCTGTTGTTGTCACACGATTCGGACAATTCTGTTTGAAAGAGAGAGAGTAACAGTACG
ATCGAACGAACTTTGCTCTGGAGATCACAGTGGGCATCATAGCATGTGGTACTAAACCCTTTCCC
GCCATTCCAGAACCTTCGATTGCTTGTTACAAAACCTGTGAGCCGTCGCTAGGACCTTGTTGTGT
GACGAAATTGGAAGCTGCAATCAATAGGAAGACAGGAAGTCGAGCGTGTCTGGGTTTTTTCAGTT
TTGTTCTTTTTGCAAACAAATCACGAGCGACGGTAATTTCTTTCTCGATAAGAGGCCACGTGCTT
TATGAGGGTAACATCAATTCAAGAAGGAGGGAAACACTTCCTTTTTCTGGCCCTGATAATAGTAT
GAGGGTGAAGCCAAAATAAAGGATTCGCGCCCAAATCGGCATCTTTAAATGCAGGTATGCGATAG
TTCCTCACTCTTTCCTTACTCACGAGTAATTCTTGCAAATGCCTATTATGCAGATGTTATAATAT
CTGTGCGTCTTGAGTTGAAGTCAGGAATCTAAAATAAAAATTAAGGTTAATAAAAAGAGGAAAGA
AAAAAAAATTAATCGATTTACAGAAACTTGCACACTAAAAATACACAACTAAAAGCAATTACAGT
ATGGGAAGTCATCGACGTTATCTCTACTATAGTATATTATCATTTCTATTATTATCCTGCTCAGT
GGTACTTGCAAAACAAGATAAGACCCCATTCTTTGAAGGTACTTCTTCGAAAAATTCGCGTCTCC
CCAAACGGGTAGTCCATCGTTGTAGGATACTGTATATACACCCAGTAGAGTAGGTGACTACTGTA
TGAGCATACAGTAGTGACAACCACTTACTGTATATAAATACAGTAGTGGTCATCGATACTGTATA
TAAAACCAGTAGGCAATCGTTTGTACTGTATGTACATACAGTATACCTCGCAACTACTGTATATA
AACACAGTAATTAACTTGTAATATTCTAATCAATTGACAATATTATTTAAGGACCTATTGTTTTT
TCCAATAGGTGGTTAGCAATCGTCTTACTTTCTAACTTTTCTTACCTTTTACATTTCAGCAATAT
ATATATATATATTTCAAGGATATACCATTCTAAGATCTAAAAAAatgaagaaaaccgtagtgatt
ggtgcaggttttggtggtttagctttggctatacgtctacaagctgcaggtattcctacagtgct
attggagcaaagagacaaaccaggaggaagagcttatgtttggcacgatcaaggctttacctttg
atgctggtcctacagtcatcactgatcctactgcattggaagctttgttcaccttagctggtaga
agaatggaagattatgtccgtctattgcctgtcaagccgttttacagattgtgttgggaatctgg
taaaaccctagattacgccaatgacagtgctgaactagaagctcagattacgcagtttaatccca
gagatgtcgaaggttacaggagattccttgcctattcccaagctgttttccaagagggttatctt
cgtttgggttcagttccattcctgtcctttagggatatgcttagagcaggtcctcagttgttgaa
gctacaagcatggcaaagtgtgtatcagtctgtttcgagatttatcgaggatgaacatctgagac
aagcattctcattccacagtcttctagttggaggtaatccctttaccacatcgagcatatatacg
ttgattcacgctttggaaagagaatggggagtttggtttcctgaaggtggaacaggtgctttggt
taatggtatggtgaagctattcacggatttgggtggagaaatagagctgaatgcaagagtggaag
aacttgttgtagcagacaacagagtctcacaagttagacttgctgatggtaggatcttcgataca
gatgctgtagcttcaaacgcagatgtagtgaacacttataaaaagttgttgggacatcatcctgt
tggacaaaagagagcagctgctttggagaggaaatctatgagcaactcgttgtttgtcctttact
ttgggctgaatcaaccacactcacaactagctcatcacacaatctgctttggtcctagatacaga
gagctgatagatgaaattttcactggatctgctttagcagacgatttttccctgtacttgcattc
accatgtgttactgatccctctttagcaccacctggttgtgctagcttctatgtactagcacctg
taccacatttgggtaatgctccattagattgggcacaagaaggaccgaaattgagggataggatc
ttcgactatttggaagaacgttacatgccaggtttgagatctcagttggttacacagaggatatt
cacaccagctgattttcatgatactctagatgcgcatttaggtagcgctttttccattgagccac
ttttgacgcaaagtgcttggtttagaccacacaacagagattctgacattgccaatctgtaccta
gtaggtgcaggaactcatccaggagctggtattcctggagttgtagcttctgctaaagctactgc
tagtctgatgatcgaggatttgcagtaaTCTCTGCTTTTGTGCGCGTATGTTTATGTATGTACCT
CTCTCTCTATTTCTATTTTTAAACCACCCTCTCAATAAAATAAAAATAATAAAGTATTTTTAAGG
AAAAGACGTGTTTAAGCACTGACTTTATCTACTTTTTGTACGTTTTCATTGATATAATGTGTTTT
GTCTCTCCCTTTTCTACGAAAATTTCAAAAATTGACCAAAAAAAGGAATATATATACGAAAAACT
ATTATATTTATATATCATAGTGTTTTTCGAGACC
pTDH3-FAD1-tPGK1, LexO(6x)-pLEU2m-CrtI-tACT1-
[pCCW12-CaBAR1I-tENO2, rev]
GGTCTCACAGTGCGCCGGGCAGTTTATCATTATCAATACTCGCCATTTCAAAGAATACGTAAATA
ATTAATAGTAGTGATTTTCCTAACTTTATTTAGTCAAAAAATTAGCCTTTTAATTCTGCTGTAAC
CCGTACATGCCCAAAATAGGGGGGGGGTTACACAGAATATATAACATCGTAGGTGTCTGGGTGAA
CAGTTTATTCCTGGCATCCACTAAATATAATGGAGCCCGCTTTTTAAGCTGGCATCCAGAAAAAA
AAAGAATCCCAGCACCAAAATATTGTTTTCTTCACCAACCATCAGTTCATAGGTCCATTCTCTTA
GCGCAACTACAGAGAACAGGGGCACAAACAGGCAAAAAACGGGCACAACCTCAATGGAGTGATGC
AACCTGCCTGGAGTAAATGATGACACAAGGCAATTGACCCACGCATGTATCTATCTCATTTTCTT
ACACCTTCTATTACCTTCTGCTCTCTCTGATTTGGAAAAAGCTGAAAAAAAAGGTTGAAACCAGT
TCCCTGAAATTATTCCCCTACTTGACTAATAAGTATATAAAGACGGTAGGTATTGATTGTAATTC
TGTAAATCTATTTCTTAAACTTCTTAAATTCTACTTTTATAGTTAGTCTTTTTTTTAGTTTTAAA
ACACCAAGAACTTAGTTTCGAAGGATTCTAGAACTAGTAACatgcagttgagcaaggctgctgag
atgtgttatgagataacaaactcttacttacacatagaccagaaatctcagataatagcaagtac
acaagaagcgatacggttgacaagaaaatacttactaagtgaaatttttgtacgttggagtccac
tgaatggggaaatatcattctcgtacaacggaggaaaagattgccaggtattactactgttatat
ctgagttgcttatgggaatatttcttcattaaggctcaaaattcccaattcgatttcgagtttca
aagcttccccatgcaaagacttccaactgttttcattgatcaagaagaaactttccctacattag
agaattttgtactggaaacctcagagcgatattgcctttccttatacgaatcacaaaggcaatct
ggtgcatcggtcaatatggcagacgcatttagagattttataaagatataccctgaAaccgaagc
tatagtgataggtattagacacacagacccatttggtgaagcattaaagcctattcaaagaacag
attctaactggcctgattttatgaggttgcaacctctcttacactgggacttaaccaatatatgg
agtttcttactgtattctaatgagccaatttgtggactatatggtaaaggtttcacatcaatcgg
cggaattaacaactcattgcctaacccacacttgagaaaggactccaataatccagccttgcatt
ttgaatgggaaatcattcatgcatttggcaaggacgcagaaggcgaacgtagttccgctataaac
acgtcacctatttccgtggtggataaggaaagattcagcaaataccatgacaattactatcctgg
ctggtatttggttgatgacactttagagagagcaggcaggatcaagaattaaATTGAATTGAATT
GAAATCGATAGATCAATTTTTTTCTTTTCTCTTTCCCCATCCTTTACGCTAAAATAATAGTTTAT
TTTATTTTTTGAATATTTTTTATTTATATACGTATATATAGACTATTATTTATCTTTTAATGATT
ATTAAGATTTTTATTAAAAAAAAATTCGCTCCTCTTTTAATGCCTTTATGCAGTTTTTTTTTCCC
ATTCGATATTTCTATGTTCGGGTTCAGCGTATTTTAAGTTTAATAACTCGAAAATTCTGCGTTCG
TTAAAGCTTTCGAGAAGGATATTATTTCGAAATAAACCGTGTTGTGTAAGCTTGAAGCCTTTTTG
CGCTGCCAATATTCTTATCCATCTATTGTACTCTTTAGATCCAGTATAGTGTATTCTTCCTGCCC
CAAACGGGTAGTCCATCGTTGTAGGATACTGTATATACACCCAGTAGAGTAGGTGACTACTGTAT
GAGCATACAGTAGTGACAACCACTTACTGTATATAAATACAGTAGTGGTCATCGATACTGTATAT
AAAACCAGTAGGCAATCGTTTGTACTGTATGTACATACAGTATACCTCGCAACTACTGTATATAA
ACACAGTAATTAACTTGTAATATTCTAATCAATTGACAATATTATTTAAGGACCTATTGTTTTTT
CCAATAGGTGGTTAGCAATCGTCTTACTTTCTAACTTTTCTTACCTTTTACATTTCAGCAATATA
TATATATATATTTCAAGGATATACCATTCTAAGATCTGGAAatgaagaaaaccgtagtgattggt
gcaggttttggtggtttagctttggctatacgtctacaagctgcaggtattcctacagtgctatt
ggagcaaagagacaaaccaggaggaagagcttatgtttggcacgatcaaggctttacctttgatg
ctggtcctacagtcatcactgatcctactgcattggaagctttgttcaccttagctggtagaaga
atggaagattatgtccgtctattgcctgtcaagccgttttacagattgtgttgggaatctggtaa
aaccctagattacgccaatgacagtgctgaactagaagctcagattacgcagtttaatcccagag
atgtcgaaggttacaggagattccttgcctattcccaagctgttttccaagagggttatcttcgt
ttgggttcagttccattcctgtcctttagggatatgcttagagcaggtcctcagttgttgaagct
acaagcatggcaaagtgtgtatcagtctgtttcgagatttatcgaggatgaacatctgagacaag
cattctcattccacagtcttctagttggaggtaatccctttaccacatcgagcatatatacgttg
attcacgctttggaaagagaatggggagtttggtttcctgaaggtggaacaggtgctttggttaa
tggtatggtgaagctattcacggatttgggtggagaaatagagctgaatgcaagagtggaagaac
ttgttgtagcagacaacagagtctcacaagttagacttgctgatggtaggatcttcgatacagat
gctgtagcttcaaacgcagatgtagtgaacacttataaaaagttgttgggacatcatcctgttgg
acaaaagagagcagctgctttggagaggaaatctatgagcaactcgttgtttgtcctttactttg
ggctgaatcaaccacactcacaactagctcatcacacaatctgctttggtcctagatacagagag
ctgatagatgaaattttcactggatctgctttagcagacgatttttccctgtacttgcattcacc
atgtgttactgatccctctttagcaccacctggttgtgctagcttctatgtactagcacctgtac
cacatttgggtaatgctccattagattgggcacaagaaggaccgaaattgagggataggatcttc
gactatttggaagaacgttacatgccaggtttgagatctcagttggttacacagaggatattcac
accagctgattttcatgatactctagatgcgcatttaggtagcgctttttccattgagccacttt
tgacgcaaagtgcttggtttagaccacacaacagagattctgacattgccaatctgtacctagta
ggtgcaggaactcatccaggagctggtattcctggagttgtagcttctgctaaagctactgctag
tctgatgatcgaggatttgcagtaaTCTCTGCTTTTGTGCGCGTATGTTTATGTATGTACCTCTC
TCTCTATTTCTATTTTTAAACCACCCTCTCAATAAAATAAAAATAATAAAGTATTTTTAAGGAAA
AGACGTGTTTAAGCACTGACTTTATCTACTTTTTGTACGTTTTCATTGATATAATGTGTTTTGTC
TCTCCCTTTTCTACGAAAATTTCAAAAATTGACCAAAAAAAGGAATATATATACGAAAAACTATT
ATATTTATATATCATAGTGT[ATTTTTCAAACTGCAAATTCAAGAAAAAGCCACGCGTGTGCACC
TTTTTTTTCCCCTTCCAGTGCATTATGCAATAGACAGCACGAGTCTTTGAAAAAGTAACTTATAA
AACTGTATCAATTTTTAAACCTAAATAGATTCATAAACTATTCGTTAATATAAAGTGTTCTAAAC
TATGATGAAAAAATAAGCAGAAAAGACTAATAATTCTTAGTTAAAAGCACTCTCGAGTTAACTAG
Tttaacaatactgagggttgcttgtgacggaagtaatcttatcatcacttgtgctgccgctcgtt
aggttgctagaggtaccctcactgatagaaaaggatgaataggtgctttggacctctgtcggtgc
aacggttaccgtggtttctgattccgcgatatatataacgctataagtgtcctcaacatcgggca
cgtctactgtcggtactggtagctggtaaccattctccacagcgacgacgttcgcctctccgtta
gaattcttatatttaccgaaatatatttgtgcgttgtcgtagtcaaatatagcatagtggtactt
aaaaatcaaatttggcagcagaccttcataggcgttatagcctatatcgcatgaagctacattta
aggctacgaatgtagttgagttcctttcaatggtatcgccgactatgtcgaccagctggaagcca
atgtcgtacccttgtactgaaaatgttatatttaacccctctatgtacttgatgtcgaaatatat
attgccattgtcatcctggatgtagtcgtcgcctaaaattttaaggatgttattaaggaccggag
ttggggggacgaaaccgttagtgccactatcgatttggtaaagcgtcttctggttgctaacgatg
gtgtctcccaacttgatctgatttactgtgaacgttggcatcaaccagtagtcaagctggtttat
gaaggatatcactttgataaagggcgctcttactaaaggcccgtctatgatgttcgtaattatcc
ctccgaagataatggacgggttctgatcctgaccattgaatgagaatagaacacttttcgtataa
ccctggtctttaagtagataggggaagttactgtaattcgttttaaaaagttcttgacgagtaaa
tccaatgcctagtataccggaaaaactgtccatatcagaaatattggccagtcccatagatagat
tagacagtttccagtcgttactcctaatagtttcattgatgaagtataccgggaactgtcccccg
tatccatatttcataattgcggtctggttggagttatagaaacttgatgacgcagagaagttgta
tatggagtcaactatcataagatcgctacttcctgaatcgactataacgttagatatgaactggg
gcggcgtccctaacgctaaattctctatgtaatacatgtgctcatcggatatcgtaaggtctaat
ttcaccgcaccgccggtattggatacggccacggctagggatgggacaagaagtgaaagaatagt
aaataacatACTAGTGATCTTTATTGATATAGTGTTTAAGCGAATGACAGAAGATTAATTTCTTG
GTATGTTAGGAAAGAATAAAGGAGAATAAGAATAATTAGAACAATGTAGGATGGAAAGAAAGATT
ATCAAGCATGCCGACTTTATATACTTGAACGGAGGCAAAGGATGCAAAATTTTCTCACATTTCTT
TCTGCCGTTATGTTGGAAGTAAGACTCCCATTATCGCAATACTGCAACACGAATATGCAAAATTT
GCTGAGTTATCGCAGATAGTTGTTGCAAAGATAGCGGCGTAGGTGGCCGCGAAATGGGAAATTCC
AAAACAAACGGTTTTTTTACTCCTGAGAAATACTTGTACGGGATAATCCAGGGCCTACCACCCAC
GCTTCGAGGATTGGCTTTTATTTTTTTTTTTTTGGTGGCGTTTTATTTCTTTCCCGCTTTCTGGG
ACTTGTGCGGAGTTTTGAGAGGGGCGCGCGGCAAAGGATTCCCAAAACGGAAATCAGACGCCAAT
AGCCAGCACTCAAAGCAGTTCTGGACCCATTCCGATTTTCCCATTTGGTTCTTGCGCGTGCTGAT
TCCGACACGCGCGTCTATAAATAGCATGAAGTATCCGCACACCGCAGCGTTAGTGAGGTGAGGGT
GGCAGCAAGCTAATTCCCGCATCTGGAATCTGAACTGCCCCTTTTGGACTAACCGTGTGGTTCAT
GGGTGTTTTCGAGACC
pTDH3-FAD1-tPGKI, LexO(6x)-pLEU2m-CrtI-tACT1-
[pCCW12-CaBAR1-tENO2, rev]
GGTCTCACAGTGCGCCGGGCAGTTTATCATTATCAATACTCGCCATTTCAAAGAATACGTAAATA
ATTAATAGTAGTGATTTTCCTAACTTTATTTAGTCAAAAAATTAGCCTTTTAATTCTGCTGTAAC
CCGTACATGCCCAAAATAGGGGGGGGGTTACACAGAATATATAACATCGTAGGTGTCTGGGTGAA
CAGTTTATTCCTGGCATCCACTAAATATAATGGAGCCCGCTTTTTAAGCTGGCATCCAGAAAAAA
AAAGAATCCCAGCACCAAAATATTGTTTTCTTCACCAACCATCAGTTCATAGGTCCATTCTCTTA
GCGCAACTACAGAGAACAGGGGCACAAACAGGCAAAAAACGGGCACAACCTCAATGGAGTGATGC
AACCTGCCTGGAGTAAATGATGACACAAGGCAATTGACCCACGCATGTATCTATCTCATTTTCTT
ACACCTTCTATTACCTTCTGCTCTCTCTGATTTGGAAAAAGCTGAAAAAAAAGGTTGAAACCAGT
TCCCTGAAATTATTCCCCTACTTGACTAATAAGTATATAAAGACGGTAGGTATTGATTGTAATTC
TGTAAATCTATTTCTTAAACTTCTTAAATTCTACTTTTATAGTTAGTCTTTTTTTTAGTTTTAAA
ACACCAAGAACTTAGTTTCGAAGGATTCTAGAACTAGTAACatgcagttgagcaaggctgctgag
atgtgttatgagataacaaactcttacttacacatagaccagaaatctcagataatagcaagtac
acaagaagcgatacggttgacaagaaaatacttactaagtgaaatttttgtacgttggagtccac
tgaatggggaaatatcattctcgtacaacggaggaaaagattgccaggtattactactgttatat
ctgagttgcttatgggaatatttcttcattaaggctcaaaattcccaattcgatttcgagtttca
aagcttccccatgcaaagacttccaactgttttcattgatcaagaagaaactttccctacattag
agaattttgtactggaaacctcagagcgatattgcctttccttatacgaatcacaaaggcaatct
ggtgcatcggtcaatatggcagacgcatttagagattttataaagatataccctgaAaccgaagc
tatagtgataggtattagacacacagacccatttggtgaagcattaaagcctattcaaagaacag
attctaactggcctgattttatgaggttgcaacctctcttacactgggacttaaccaatatatgg
agtttcttactgtattctaatgagccaatttgtggactatatggtaaaggtttcacatcaatcgg
cggaattaacaactcattgcctaacccacacttgagaaaggactccaataatccagccttgcatt
ttgaatgggaaatcattcatgcatttggcaaggacgcagaaggcgaacgtagttccgctataaac
acgtcacctatttccgtggtggataaggaaagattcagcaaataccatgacaattactatcctgg
ctggtatttggttgatgacactttagagagagcaggcaggatcaagaattaaATTGAATTGAATT
GAAATCGATAGATCAATTTTTTTCTTTTCTCTTTCCCCATCCTTTACGCTAAAATAATAGTTTAT
TTTATTTTTTGAATATTTTTTATTTATATACGTATATATAGACTATTATTTATCTTTTAATGATT
ATTAAGATTTTTATTAAAAAAAAATTCGCTCCTCTTTTAATGCCTTTATGCAGTTTTTTTTTCCC
ATTCGATATTTCTATGTTCGGGTTCAGCGTATTTTAAGTTTAATAACTCGAAAATTCTGCGTTCG
TTAAAGCTTTCGAGAAGGATATTATTTCGAAATAAACCGTGTTGTGTAAGCTTGAAGCCTTTTTG
CGCTGCCAATATTCTTATCCATCTATTGTACTCTTTAGATCCAGTATAGTGTATTCTTCCTGCCC
CAAACGGGTAGTCCATCGTTGTAGGATACTGTATATACACCCAGTAGAGTAGGTGACTACTGTAT
GAGCATACAGTAGTGACAACCACTTACTGTATATAAATACAGTAGTGGTCATCGATACTGTATAT
AAAACCAGTAGGCAATCGTTTGTACTGTATGTACATACAGTATACCTCGCAACTACTGTATATAA
ACACAGTAATTAACTTGTAATATTCTAATCAATTGACAATATTATTTAAGGACCTATTGTTTTTT
CCAATAGGTGGTTAGCAATCGTCTTACTTTCTAACTTTTCTTACCTTTTACATTTCAGCAATATA
TATATATATATTTCAAGGATATACCATTCTAAGATCTGGAAatgaagaaaaccgtagtgattggt
gcaggttttggtggtttagctttggctatacgtctacaagctgcaggtattcctacagtgctatt
ggagcaaagagacaaaccaggaggaagagcttatgtttggcacgatcaaggctttacctttgatg
ctggtcctacagtcatcactgatcctactgcattggaagctttgttcaccttagctggtagaaga
atggaagattatgtccgtctattgcctgtcaagccgttttacagattgtgttgggaatctggtaa
aaccctagattacgccaatgacagtgctgaactagaagctcagattacgcagtttaatcccagag
atgtcgaaggttacaggagattccttgcctattcccaagctgttttccaagagggttatcttcgt
ttgggttcagttccattcctgtcctttagggatatgcttagagcaggtcctcagttgttgaagct
acaagcatggcaaagtgtgtatcagtctgtttcgagatttatcgaggatgaacatctgagacaag
cattctcattccacagtcttctagttggaggtaatccctttaccacatcgagcatatatacgttg
attcacgctttggaaagagaatggggagtttggtttcctgaaggtggaacaggtgctttggttaa
tggtatggtgaagctattcacggatttgggtggagaaatagagctgaatgcaagagtggaagaac
ttgttgtagcagacaacagagtctcacaagttagacttgctgatggtaggatcttcgatacagat
gctgtagcttcaaacgcagatgtagtgaacacttataaaaagttgttgggacatcatcctgttgg
acaaaagagagcagctgctttggagaggaaatctatgagcaactcgttgtttgtcctttactttg
ggctgaatcaaccacactcacaactagctcatcacacaatctgctttggtcctagatacagagag
ctgatagatgaaattttcactggatctgctttagcagacgatttttccctgtacttgcattcacc
atgtgttactgatccctctttagcaccacctggttgtgctagcttctatgtactagcacctgtac
cacatttgggtaatgctccattagattgggcacaagaaggaccgaaattgagggataggatcttc
gactatttggaagaacgttacatgccaggtttgagatctcagttggttacacagaggatattcac
accagctgattttcatgatactctagatgcgcatttaggtagcgctttttccattgagccacttt
tgacgcaaagtgcttggtttagaccacacaacagagattctgacattgccaatctgtacctagta
ggtgcaggaactcatccaggagctggtattcctggagttgtagcttctgctaaagctactgctag
tctgatgatcgaggatttgcagtaaTCTCTGCTTTTGTGCGCGTATGTTTATGTATGTACCTCTC
TCTCTATTTCTATTTTTAAACCACCCTCTCAATAAAATAAAAATAATAAAGTATTTTTAAGGAAA
AGACGTGTTTAAGCACTGACTTTATCTACTTTTTGTACGTTTTCATTGATATAATGTGTTTTGTC
TCTCCCTTTTCTACGAAAATTTCAAAAATTGACCAAAAAAAGGAATATATATACGAAAAACTATT
ATATTTATATATCATAGTGT[ATTTTTCAAACTGCAAATTCAAGAAAAAGCCACGCGTGTGCACC
TTTTTTTTCCCCTTCCAGTGCATTATGCAATAGACAGCACGAGTCTTTGAAAAAGTAACTTATAA
AACTGTATCAATTTTTAAACCTAAATAGATTCATAAACTATTCGTTAATATAAAGTGTTCTAAAC
TATGATGAAAAAATAAGCAGAAAAGACTAATAATTCTTAGTTAAAAGCACTCTCGAGTTAACTAG
Tttaacaatactgagggttgcttgtgacggaagtaatcttatcatcacttgtgctgccgctcgtt
aggttgctagaggtaccctcactgatagaaaaggatgaataggtgctttggacctctgtcggtgc
aacggttaccgtggtttctgattccgcgatatatataacgctataagtgtcctcaacatcgggca
cgtctactgtcggtactggtagctggtaaccattctccacagcgacgacgttcgcctctccgtta
gaattcttatatttaccgaaatatatttgtgcgttgtcgtagtcaaatatagcatagtggtactt
aaaaatcaaatttggcagcagaccttcataggcgttatagcctatatcgcatgaagctacattta
aggctacgaatgtagttgagttcctttcaatggtatcgccgactatgtcgaccagctggaagcca
atgtcgtacccttgtactgaaaatgttatatttaacccctctatgtacttgatgtcgaaatatat
attgccattgtcatcctggatgtagtcgtcgcctaaaattttaaggatgttattaaggaccggag
ttggggggacgaaaccgttagtgccactatcgatttggtaaagcgtcttctggttgctaacgatg
gtgtctcccaacttgatctgatttactgtgaacgttggcatcaaccagtagtcaagctggtttat
gaaggatatcactttgataaagggcgctcttactaaaggcccgtctatgatgttcgtaattatcc
ctccgaagataatggacgggttctgatcctgaccattgaatgagaatagaacacttttcgtataa
ccctggtctttaagtagataggggaagttactgtaattcgttttaaaaagttcttgacgagtaaa
tccaatgcctagtataccggaaaaactgtccatatcagaaatattggccagtcccatagatagat
tagacagtttccagtcgttactcctaatagtttcattgatgaagtataccgggaactgtcccccg
tatccatatttcataattgcggtctggttggagttatagaaacttgatgacgcagagaagttgta
tatggagtcaactatcataagatcgctacttcctgaatcgactataacgttagatatgaactggg
gcggcgtccctaacgctaaattctctatgtaatacatgtgctcatcggatatcgtaaggtctaat
ttcaccgcaccgccggtattggatacggccacggctagggatgggacaagaagtgaaagaatagt
aaataacatACTAGTGATCTTTATTGATATAGTGTTTAAGCGAATGACAGAAGATTAATTTCTTG
GTATGTTAGGAAAGAATAAAGGAGAATAAGAATAATTAGAACAATGTAGGATGGAAAGAAAGATT
ATCAAGCATGCCGACTTTATATACTTGAACGGAGGCAAAGGATGCAAAATTTTCTCACATTTCTT
TCTGCCGTTATGTTGGAAGTAAGACTCCCATTATCGCAATACTGCAACACGAATATGCAAAATTT
GCTGAGTTATCGCAGATAGTTGTTGCAAAGATAGCGGCGTAGGTGGCCGCGAAATGGGAAATTCC
AAAACAAACGGTTTTTTTACTCCTGAGAAATACTTGTACGGGATAATCCAGGGCCTACCACCCAC
GCTTCGAGGATTGGCTTTTATTTTTTTTTTTTTGGTGGCGTTTTATTTCTTTCCCGCTTTCTGGG
ACTTGTGCGGAGTTTTGAGAGGGGCGCGCGGCAAAGGATTCCCAAAACGGAAATCAGACGCCAAT
AGCCAGCACTCAAAGCAGTTCTGGACCCATTCCGATTTTCCCATTTGGTTCTTGCGCGTGCTGAT
TCCGACACGCGCGTCTATAAATAGCATGAAGTATCCGCACACCGCAGCGTTAGTGAGGTGAGGGT
GGCAGCAAGCTAATTCCCGCATCTGGAATCTGAACTGCCCCTTTTGGACTAACCGTGTGGTTCAT
GGGTGTTTTCGAGACC

### Plasmid Cloning and Assembly:

Restriction enzyme digest—Restriction enzymes and buffers were purchased from New England Biolabs (NEB) (Ipswich, MA, USA) and manufacturer's protocol was followed. In a typical restriction enzyme digest reaction, a plasmid of interest (total amount 1, μg) was mixed with selected restriction enzyme and appropriate buffer at 50 μL total volume. Reaction was incubated at specified temperature for at least one hour. Then, the reaction solution was mixed with purple Gel Loading Dye (NEB) and run in 1% agarose gel with 1:100,000 ethidium bromide alongside appropriate DNA ladder for 25-30 min at 120 V. Gel was imaged using ChemiDoc MP Imaging System (Bio-Rad Laboratories, Inc.) with Ethidium Bromide Optimal Auto-Exposure setting. DNA fragment of the correct band size was cut out and gel extracted using QIAquick Gel Extraction Kit (QiaGen) by following manufacturer's protocol. DNA concentration was determined using Infinite 200 Pro plate reader (Tecan).

Ligation—Ligase and buffers were purchased from New England Biolabs (NEB) (Ipswich, MA, USA) and manufacturer's protocol was followed. In a typical ligation reaction, DNA fragments with complementary 4-bp sticky ends were mixed in 1:1 molar ratio (total largest fragment amount 50 ng) with selected ligase and appropriate buffer at 20 μL total volume. Reaction was incubated at 16° C. overnight. Then, the reaction solution was diluted with water in 1:3 ratio and electrocompetent E. coli cells were transformed via electroporation.

Gibson assembly—Gibson Master Mix purchased from New England Biolabs (NEB) (Ipswich, MA, USA) and manufacturer's protocol was followed. In a typical Gibson assembly reaction, DNA fragments with 20-30-bp homologous overlaps were mixed in 1:1 molar ratio (total largest fragment amount 20 ng) with 2× Gibson Master Mix at 5 μL total volume. Reaction was incubated at 50° C. for at least 30 min. Then, the reaction solution was diluted with water in 1:3 ratio and electrocompetent E. coli cells were transformed via electroporation.

Golden Gate assembly—A Golden Gate assembly reaction mixture was prepared as follows: appropriate volume of each insert plasmid in 3:1 ratio with integration plasmid set to 10 ng, 0.75 μL T4 DNA Ligase buffer, 0.75 μL T4 DNA Ligase (400 U/μL), 0.45 μL BsaI restriction enzyme (20 U/μL), 0.075 μL BSA (20 mg/mL) and water to bring the final volume to 7.5 μL. Reaction mixtures were incubated in a thermocycler according to the following program: 50-60 cycles of digestion and ligation (37° C. for 3 min, 16° C. for 4 min) followed by a final digestion step (37° C. for 60 min), and a heat inactivation step (80° C. for 5 min). The mixture is then diluted 1:3 with water and E. coli is transformed via electroporation and plated on LB plate supplemented with appropriate antibiotic. Correctly assembled plasmids are screened and verified using colony PCR and Sanger sequencing. Correctly transformed colony was grown in LB broth supplemented with appropriate antibiotic overnight and stocked with 20% glycerol at −80° C.

### CRISPR/Cas9 Yeast Genomic Manipulation:

For CRISPR/Cas9-guided genomic manipulation, yeast strain is chemically transformed with two components: Cas9/gRNA plasmid and linear DNA repair fragment. The plasmid constitutively expresses Cas9 and appropriate gRNA targeting yeast genomic region of interest. Linear DNA fragment contains 500-bp upstream and 500-bp downstream homology around targeted genomic site and a desired insert sequence in between.

For genomic deletion, DNA repair fragment is generated by PCR amplifying 500 bp upstream and 500 bp downstream from desired genomic site for deletion with 30-bp homologous overlaps for Gibson assembly of those two fragments. After Gibson assembly, a 1,000-bp repair fragment (500 bp upstream+500 bp downstream) is amplified and transformed along with appropriate Cas9/gRNA plasmid using modified lithium acetate method. For genomic insertion, DNA repair fragment is generated by PCR amplifying 500 bp upstream and 500 bp downstream from desired genomic site for replacement and insertion with 30-bp homologous overlaps for Gibson assembly of those two fragments with appropriate insert in between. After Gibson assembly, a +1,000-bp repair fragment (500 bp upstream+insert+500 bp downstream) is amplified and transformed along with appropriate Cas9/gRNA plasmid using modified lithium acetate method.

For genomic insertion using auxotrophic selection markers, DNA repair fragment is generated by Golden Gate assembling desired inserts into appropriate integration plasmid containing desired integration site homology regions and auxotrophic selection marker. After Golden Gate assembly, integration plasmid is digested with NotI for linearization and used for standard lithium acetate transformation as is.

CRISPR gRNA Design and Cloning:

CRISPR/Cas9 20-bp gRNA sequence for particular yeast genomic site was determined using Atum.bio CRISPR gRNA Design tool, or Benchling.com CRISPR Guide Design tool. Ideally, gRNA genomic region site is selected as close to the area targeted for modification as possible. Then, two 60-bp complementary DNA oligos were ordered which had 20-bp upstream and 20-bp downstream homology with digested gRNA/Cas9 plasmid backbone and 20-bp gRNA sequence. DNA oligos were annealed, diluted 1:1,000 with water and then assembled into NotI-digested plasmids pSB36, pTC07 or pTC163 using Gibson assembly protocol.

### Results

The parent strain from an optimized biosensing strain yWS890 reported and developed by Shaw et al. (“Engineering a Model Cell for Rational Tuning of GPCR Signaling,” Cell, vol. 177, pp. 1-15, 2019) was developed. yWS890 is lab strain S. cerevisiae a-mating type haploid BY4741 with deletions and refactoring within pheromone signaling pathway for enhanced biosensing. The strain has deleted genes which otherwise interfere with orthogonal and robust signaling within this pathway. The strain has deleted native mating pheromones Mfa1, Mfa2, Mf(alpha)1 and Mf(alpha)2, alpha-factor protease Bar1, cell cycle arrest inducer Far1, negative regulator of Gpa1 Sst2 and glucose sensing GPCR Gpr1 and associated G protein α-subunit Gpa2. Additionally, Ste2 and Gpa1 signaling components were deleted and then reintegrated with selected constitutive promoters for fine-tuned signaling. Finally, native transcription factor Ste12 was replaced in this strain by synthetic transcription factor which is a fusion of native pheromone-response domain of Ste12 (PRD) and LexA bacterial repressor protein that can activate modular synthetic promoters containing LexA operator sequences (LexO) (Shaw et al.). This strain also has fine-tuned and integrated two components to mix-and-match as part of the biosensing component toolkit—native pheromone GPCR Ste2 and inducible sfGFP as a readout. In order to engineer this parent strain, Ste2, sfGFP, LEU2 and URA3 was deleted in the yWS890 genome along with their corresponding promoters and terminators using Cas9/gRNA technology. LEU2 and URA3 selection markers were deleted, since their auxotrophy will be needed for the CRISPR/Cas9 genomic integration selections. The engineered parent strain yTC370 now has refactored and fine-tuned intracellular signaling pathway and is only missing the GPCR and inducible readout as minimal biosensing components.

Next, a modular and interchangeable toolkit was set up for simple mixing-and-matching of desired biosensing components and integrating them into “empty” parent strain yTC370. Amodification of yeast MoClo Toolkit developed by Lee et al. (M. E. Lee, W. C. Deloache, B. Cervantes, and J. E. Dueber, “A Highly Characterized Yeast Toolkit for Modular, Multipart Assembly,” ACS Synth. Biol., vol. 4, pp. 975-986, 2015) was created that includes a library of four different types of transcription units which can be assembled together into a single integration plasmid using Golden Gate assembly. The transcription unit types are divided based on the core function which they bring. The transcription unit types are: constitutive GPCR expression, inducible readout expression, inducible peptide secretion and constitutive peptide protease expression. GPCR and inducible readout are the minimal components which have to be integrated into parent strain in order to constitute a functional biosensor. Inducible peptide secretion and constitutive protease expression are supplementary components which can be used for engineering multi-membered communities and amplification loops, and for modulating response sensitivity and auto-activation, respectively.

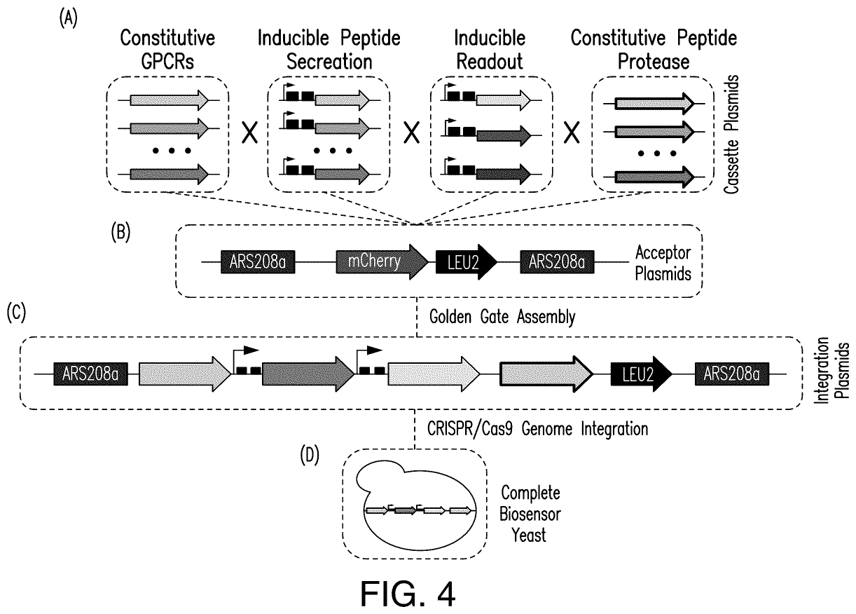

Each type of transcriptional unit contains different GPCRs, readouts, secreted peptides and proteases which can be combined using Golden Gate assembly. The ORFs of all four components are codon-optimized for S. cerevisiae expression and they do not contain internal BsaI restriction sites, as they would impede Golden Gate assembly. Further on, the constitutive GPCR expression cassette plasmids were cloned with strong characterized constitutive promoter pGPD (Lee et al.) and native Ste2 terminator tSte2, as these promoter and terminator pairs were used and reported by Billerbeck et al. (A scalable peptide-GPCR language for engineering multicellular communication,” Nature Communications, vol. 9, no. 1, p. 5057, 2018) for GPCR expression and it was experimentally determined that strong GPCR expression leads to the stronger and more sensitive response compared to weaker promoters (Shaw et al.). Inducible readout cassette plasmids were cloned with pLEU2m core promoter and six copies of upstream activating sequences [LexO(6×)-pLEU2m] which are strongly and orthogonally activated by engineered fusion of the full-length bacterial LexA repressor with the Ste12 Pheromone-Responsive Domain within the parent strain. For terminator, a standard characterized tTDH1 was selected. Inducible peptide secretion cassette plasmids were cloned with pALD6m core promoter and four copies of upstream activating sequences [LexO(4×)-pALD6m]. For terminator, a standard characterized tCYC1 was selected. Finally, constitutive protease expression cassette plasmids were cloned with three different characterized promoters of different expression strength (pRAD27 for low, pRPL18B for medium and pCCW12 for high) (Lee et al.), as it is expected that lower expression levels of particular proteases may be sufficient to prevent basal peptide secretion, while for some peptides, a higher protease expression may be needed. Since proteases generally attenuate overall peptide response, their expression level should be balanced based on the desired outcome of the engineered biosensing strains. Ultimately, a library of cassette plasmids with relevant GPCRs, readouts, secreted peptides and proteases was created which can be mixed-and-matched using Golden Gate assembly into an acceptor plasmid (FIG. 4A).

Next, acceptor plasmids were designed and cloned which are going to be used for straightforward and efficient integration of assembled biosensing components into parent yeast strain. The acceptor plasmid contains constitutive mCherry expression cassette with appropriate Golden Gate assembly overlaps in order for mCherry to be cut out and replaced by correctly assembled transcription units from cassette plasmids which gives a simple and straightforward E. coli colony selection criteria when screening for correctly assembled acceptor plasmids. Additionally, the acceptor plasmid contains LEU2, HIS3 or URA3 selection markers for yeast genomic integration selection and the plasmid contains 500-bp upstream and downstream homology sequences for integration into ARS208a, HO or LEU2 locus. Immediately upstream and downstream from the whole construct is rare NotI restriction site for linearization of the integration fragment which greatly improves integration efficiency (FIG. 4B). After Golden Gate assembly, the correctly assembled integration plasmid (FIG. 4C) is linearized and transformed into parent yeast strain along with appropriate Cas9/gRNA plasmid depending on the selected genome integration site. Once integrated, the parent yeast strain then manifests all new functional biosensing components (FIG. 4D). It was decided to always integrate the biosensing components into the genome, rather than having them on episomal plasmids maintained with auxotrophic selection markers, due to more stable and less variable expression of the integrated components (Billerbeck et al. and Lee et al.). With developed biosensing component integration toolkit, further engineering and optimization of the A. fumigatus biosensor was performed.

### Example 3: Improvement of A. fumigatus Biosensor Strain Signal-to-Noise Ratio

This example discloses the improvement in the signal-to-noise ration of the A. fumigatus biosensor shown in Example 1.

### Materials and Methods

General protocol: Glycerol stocked strains were streaked out on appropriate plate and incubated for two days at 30° C. Three colonies were picked, replated on fresh plate, incubated for one day and then stored at 4° C. For activation assays, a small scoop of yeast patch from the three colonies was resuspended in ≈4 mL appropriate media and incubated overnight. Overnight cells were seeded at an OD600 of 0.15 (as measured in 96-well plate at 200-μL volume) in SC/Glc media after washing twice with sterile MiliQ H2O. Activity and response to different concentrations of ligands were measured in black 96-well microtiter plates with transparent bottom in 200 μL total volume, cultured at 30° C. and 800 rpm, unless otherwise stated. Culture turbidity (OD600) and fluorescence were measured at different time points using an Infinite 200 Pro plate reader (Tecan). To account for the optical density values exceeding the linear range of the photo detector, all optical density values were corrected with the following formula to yield true optical density values:

$${A}_{true} = \frac{k * {A}_{meas}}{{A}_{sat} - {A}_{meas}}$$

Where Asat is the photodetector saturation value, Ameas is the measure optical density, and k is the true optimal density at which the detector reaches half saturation of the measured optical density [63]. k value is 3.16 and Asat value is 3.57. Dose-responses were measured at different concentrations (usually eleven x-fold dilutions in H2O starting with maximal peptide concentration and H2O was used as no peptide control) of the appropriate synthetic peptide ligand. All fluorescence values were normalized by the A600, and plotted against the log (10)-converted peptide concentrations. Data were fit to a four-parameter non-linear regression model using Prism (GraphPad) in order to extract specific values for basal activation, maximal activation, EC₅₀, and the Hill coefficient. Error bars represent standard deviation (SD).

### Fluorescent Readout Measurement Parameters:

| Fluorescent Protein | Excitation (nm) [266] | Emission (nm) [266] | Gain |
|---|---|---|---|
| mCherry (RFP) | 588 | 620 | 130 |
| sfGFP | 485 | 528 | 80 |
| ymTurquoise2 | 439 | 475 | 75 |
| ytdTomato | 552 | 580 | 90 |

Gain was empirically determined for SC/Glc media in standard black 96-well plate at 200-μL volume in order to have raw fluorescence intensity value around 15 A. U.

### Results

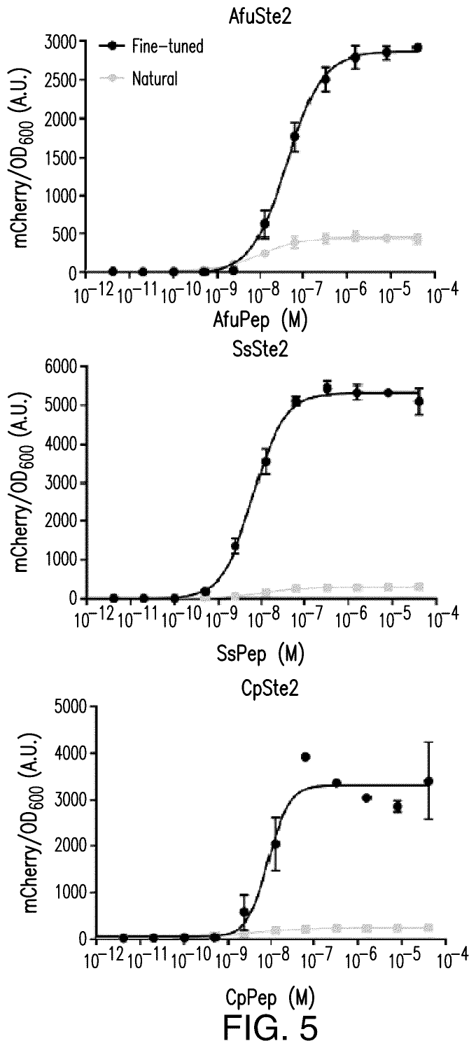

The initial A. fumigatus biosensor strain was constructed by placing a plasmid-expressed A. fumigatus pheromone GPCR AfuSte2 into a parent biosensor strain reported by Ostrov et al. This parent strain is a lab strain S. cerevisiae which simply had deleted particular pheromone signalling pathway components which would enable swapping of different fungal GPCRs for different peptide pheromone sensing and having mCherry expression under endogenous pheromone-inducible pFUS1 promoter (M. Jimenez, Low Cost Pathogen Detection with Yeast and Tools for Synthetic Multicellular Systems [Doctoral dissertation, Columbia University], 2016. arXiv: arXiv:1011). Here, the A. fumigatus biosensing was improved by implementing AfuSte2 GPCR into the biosensing parent strain yTC370. In order to directly compare the biosensing performance of the two parent strains (yMJ194 and yTC370), inducible mCherry was integrated into yTC370 and transformed the strain with AfuSte2 on a plasmid (pDR01), as it was done in parent strain yMJ194 in Example 1. Dose-response activation of each strain with AfuPep was then compared (FIG. 5).

The biosensing performance between different living biosensing yeast can be compared using dose-response activation curves. The yeast is treated with peptide ligand of interest at different concentrations and then the inducible readout is measured after incubation time. By fitting four parameter nonlinear regression with variable slope equation (Hill function) to the dose-response data, basal activation can be determined by looking at the readout intensity at ligand concentration zero, maximal activation by looking at the readout intensity which plateaus at high ligand concentration, EC₅₀ by looking at the ligand concentration where half-maximal readout intensity is reached and lastly the Hill slope shows the “steepness” of the curve at ligand concentrations around EC₅₀. The fine-tuned optimized strain showed an outstanding improvement in AfuPep biosensing performance (FIG. 5). The basal expression dropped from ˜20 A.U. to practically zero and the maximal activation increased from ˜450 A.U. to ˜2,900 A.U. effectively increasing activation fold change from 19-fold to 2,900-fold. The sensitivity in optimized strain is slightly decreased with EC₅₀ going up from ˜10 nM to ˜40 nM, however, due to enormous enhancement in the overall activation signal, optimized strain is showing higher fluorescence intensity at ˜10 nM AfuPep compared to older strain with natural signaling pathway which effectively improves LoD in the optimized strain. This signaling enhancement is not specific only for AfuSte2 GPCR and AfuPep, but also for two other GPCRs tested—SsSte2 and CpSte2 (FIG. 5). The dose-response activation of SsSte2 and CpSte2 GPCRs expressed on plasmids, transformed into yMJ194 and yTC453 and tested activation with cognate peptides SsPep and CpPep, respectively, were compared. For both GPCRs, the basal expression dropped to practically 0 A.U. For SsSte2, maximal activation increased from ˜320 A.U. to ˜5,300 A.U. and EC₅₀ decreased from ˜12 nM to ˜7 nM. For CpSte2, maximal activation increased from ˜250 A.U. to ˜3,300 AU. and EC₅₀ increased from ˜3 nM to ˜9 nM. After validating the biosensing performance of the fine-tuned strain, it was used as a parent strain for all future biosensing strains to be engineered.

### Example 4: Pheromones, GPCRs and Cross-Activation of Other Aspergillus Species

To probe receptor's specificity to other pheromone peptides of closely related Aspergillus species, additional Aspergillus pheromone receptors and cognate pheromones of interest for heterologous expression in the biosensor yeast were genome mined and literature-searched.

Materials and Methods
Aspergillus spp mating GPCRs - Codon-optimized ORFs
without internal BsaI or NotI sites:
A. clavatus; AclaSte2
ATGAACAGTACGTTCGATCCCTTCGAACAAAGCTTCACTCTACAACGTTCCGATGGTAAGACGTT
CAATACTTCTCTGGTTATCGCCGACCATAACATGGAATATATGCTGAGACTTTCTATTAACTACG
GAACCCAGTTGGGAGCATCAATCGTCCTATTACTGATACTAATGCTTCTAACTCGTCCGGAGAAG
AGGCTGAGTAGCGTATTCCTACTAAACGTGGCTGCGCTTGCATCAAACATCGTCAGGCTGGTATG
CCAGATAAGCTATTTTTCATCTGGGTTTGCGAAAACCTATGCTTTATTAGCGCATGATTTCTCCA
GGGTGGCCCAGGCTACATACGCAAGTCAAGTTATGGCATCAGTTTTTTTTACGTTGGTCTTGGTG
TGTGTGGAAATTTCACTAGTCCTACAAGTACAGGTCGTTTGCTCTAACTTGCGTAGGAGGTATAG
GCACTTACTTCTTGGTGCATCCATATTGGTAACACTTATACCGATAGGGTTTAGATTGGCTTTTA
TGGTAGAGAATTGCATCGTAATCGTTCGTAGTGGAAACATGAAGAATTACCATTGGTTGGAGTCC
GCTACGAACATTGTCGCTACTGTGTCAATATGTTTTTTTTGCGCGATATTCGTCGTAAAGCTGGG
CTTCGCAATAAAGATGAGGCGTCGTCTGGGGGTAAAAGATTTTGGCCCTATGAAGGTCATATTCA
TTATGGGCTGCCAAACCATGATCATCCCTGCCCTATTTTCTATTTGTCAATATTGGGTTAACATA
CCTGAATTTTCACACAACTTGCTGACGTTAGTAACTATCTCACTGCCTCTAAGTAGTATTTGGGC
AGGTTTCACTCTGGATAGCTCTAATAACACAGCGAACTCCACAGCTAGCCGTAGAAACCTGTGGA
ATGTGTTATCCAGCAATGGTACGGTTCAAGACAAACCGAATCTTTCTGCGTGCTCTCCGGTAGCC
TCACCAAATACGACCTGTTATAGTGACCCTTATAGCGGCAAGCCGCACCATCAGGATCTAGAGGC
TGGATTTGGCATCAGTGTGGAACACGATATTAGCATACACTCTTATAGAAAGGACGCACATAGTG
ATGTT
A. fischeri; AfiSte 2
ATGAACTCCACCTTCGACCCATGGACCCAAAACATTACTTTGACTCAATCCGACGGTACCACTGT
CATCTCCTCTTTGGCTTTGGCCGATGACTACTTGCACTACATGATTAGATTGGGTATCAACTACG
GTGCCCAATTGGGTGCTTGTGCTGTTTTGTTGTTGGTTTTGTTATTGTTGACTAGACCAGAAAAG
AGAGTTTCTTCTGTCTTCGTTTTGAACGTCGCTGCTTTGTTGGCTAACATCATCAGATTGGGTTG
TCAATTGTCCTACTTCTCTACCGGTTTCGCTAGAATGTACGCCTTGTTGGCCGGTGACTTCTCCA
GAGTCTCTCGTGGTGCTTACGCCGGTCAAGTTATGGCCTCCGTCTTCTTCACCATTGTCTTCATT
TGTGTTGAAGCTTCTTTGGTTTTGCAAGTTCAAGTCGTCTGTTCTAACTTGAGAAGACAATACAG
AATCTTGTTATTGGGTGCTTCCACTTTGGCTGCCTTGGTTCCAATTGGTGTTCGTTTGACTTACT
CCGTTTTAAACTGTATGGTTATTATGCACGCTGGTACTATGGACCACTTGGATTGGTTGGAATCT
GCTACCAACATCGTTACTACCGTTTCTATTTGTTTCTTCTGTGCTGTTTTCGTTGTCAAATTAGG
TTTGGCTATCAAGATGAGAAAGCGTTTGGGTGTCAAACAATTCGGTCCAATGAGAGTTATCTTCA
TCATGGGTTGTCAAACCATGACCATCCCAGCTATTTTCGCTATTTGTCAATACTTCTCTAGAATT
CCAGAATTTTCTCATAACGTTTTGACTTTGGTTATCATCTCTTTGCCATTGTCTTCTATCTGGGC
CGGTTTTGCTTTGGTCCAAGCCAACTCTACCGCCAGATCTACCGAATCTAGACATCATTTGTGGA
ACATTTTGTCTTCCGATGGTGCTACCAGAGACAAGCCATCCCAATGTGTTTCTTCTCCAATGACC
TCTCCAACCACTACCTGTTACTCCGAACAATCCACCTCTAAGCCACAACAAGACCCAGAAAACGG
TTTTGGTATTTCTGTTGCCCACGATATTTCCATCCACTCTTTCAGAAAGGACGCCCACGGTGATA
TT
A. flavus, A. oryzae; AoSte2
ATGGACTCTAAGTTCGACCCATACTCTCAAAACTTGACTTTCCACGCTGCTGACGGTACCCCATT
TCAAGTTCCAGTCATGACCTTGAACGACTTTTACCAATACTGTATTCAAATTTGTATCAACTACG
GTGCTCAATTCGGTGCTTCCGTCATCATTTTCATTATCTTGTTGTTATTGACTAGACCAGACAAA
AGAGCTTCTTCTGTTTTCTTCTTAAACGGTGGTGCCTTGTTGTTGAACATGGGTAGATTGTTGTG
TCACATGATTTACTTCACTACTGACTTCGTCAAGGCTTACCAATACTTCTCTTCTGATTACTCTA
GAGCCCCAACCTCTGCCTACGCTAACTCCATTTTGGGTGTCGTCTTGACCACCTTGTTGTTGGTT
TGTATCGAAACCTCCTTGGTTTTACAAGTCCAAGTCGTCTGTGCTAACTTGAGACGTAGATACAG
AACCGTCTTATTGTGTGTTTCTATCTTGGTCGCCTTGATCCCAGTCGGTTTGAGATTGGGTTACA
TGGTTGAAAACTGTAAGACTATTGTTCAAACTGATACCCCATTGTCTTTGGTTTGGTTGGAATCT
GCTACTAACATCGTCATTACCATCTCCATCTGTTTCTTCTGTTCTATCTTCATCATCAAGTTGGG
TTTCGCCATTCACCAAAGAAGAAGATTGGGTGTCAGAGATTTCGGTCCAATGAAGGTCATTTTCG
TCATGGGTTGTCAAACTTTGACTGTTCCAGCTTTGTTGTCTATTTTGCAATACGCTGTCTCTGTC
CCAGAATTGAACTCTAACATTATGACTTTGGTTACTATCTCTTTGCCATTGTCCTCCATTTGGGC
TGGTGTTTCTTTGACCCGTTCTTCCTCCACCGAAAACTCTCCATCCAGAGGTGCTTTGTGGAACC
GTTTGACCGACTCTACCGGTACCAGATCTAACCAAACCTCTTCCACCGACACCGCCGTCGCTATG
ACCTACCCATCTAACAAGTCTTCTACTGTCTGTTACGCCGATCAATCTTCTGTCAAGAGACAATA
CGATCCAGAACAAGGTCACGGTATCTCTGTTGAACACGATGTTTCTGTCCACTCCTGTCAAAGAT
TG
A. fumigatus; AfuSte2
ATGAACTCAACATTTGACCCCTGGACACAAAACATTACACTAACACAAAGTGACGGGACTACAGT
CGTCAGTTCACTTGCACTGGCTGATGACTACCTGCATTACATGATCAGGCTAGGAATTAACTATG
GGGCACAGTTAGGCGCCTGCGCCGTACTATTCCTTGTCCTACTTTTGCTTACCAGGCCCGAAAAG
AGGGTATCTAGTGTGTTCGTTTTGAACGTGAGTGCACTTTTAGCCAATATAATAAGGCTAGGTTG
TCAACTTTCCTACTTTTCCACGGGCTTCGCGCGTATGTACGCATTGTTGGCCGGTGACTTCAGCA
GAGTGAGCCGTGGAGCCTATGCGGGTCAAGTAATGGCTTCAGTATTTTTTACAATAGTTCTGATT
TGCGTCGAAGCCTCTTTAGTCCTGCAAGTGCAGGTCGTGTGCTCCAATCTTCTGCGTCAGTATAG
GATTCTTCTATTGGGCGCTTCCACCCTGGCAGCTCTGGTGCCCATAGGCGTACGTCTAACATACA
GTGTGATGAATTGCATGGTGATCATGCACGCTGGCACAATGGACCATCTGGACTGGTTAGAGAGC
GCAACAAATATCGTTACGACTGTCAGTATTTGTTTCTTTTGCGCGGTGTTCGTAGTGAAGTTGGG
CCTGGCTATAAAAATGAGGAAGAGATTGGGTGTCAAGAAGTTTGGACCGATGAGAGTAATATTCA
TCATGGGCTGCCAAACTATGACCATCCCTGCCGTGTTCGCAATCTGTCAGTATTTCAGTAGGATT
CCAGAATTCTCTCATAACGTTTTGACCTTAGTAATCATTTCCCTGCCCTTATCAAGTATATGGGC
CGGGTTCGCTTTGGATCAAGCGAACTCTACGGCTCGTAGTACAGAATCTCGTCACCACCTGTGGA
ATATTCTTTCATCAGATGGTGCGACAAGAGATAAACCTTCCCAGTGTGCAAATAGTCCAATGACA
AGTCCGAATACGACCTGTTATTCCGAACAGTCAACGTCCAAGCCTCAGCAAGATCCCGAGAACGG
GTTTGGGATTAGCGTAGCTCATGATATCAGTATCCACTCCTTCCAAAAAGGGGGGCACGGCGACA
TT
A. lentulus; AleSte2
ATGAATTCAACGTTCGATCCGTGGACTCAAAATATAACGCTTACTCAGAGTGATGGCACGACAGT
TATATCATCTCTTGCGCTAGCGGACGACTACCTGCATTACATGATACGTTTATCCATTAATTACG
GTGCACAATTAGGAGCTTGCGCTGTCCTACTTCTAGTCCTTTTGCTGTTGACAAGACCGGAAAAA
AGGGTAAGTAGCGTATTTGTACTGAATGTAGCTGCTCTACTTGCGAATATAATAAGACTTGGATG
TCAACTTTCATACTTTTCTACTGGTTTCGCACGTATGTATGCCCTTCTAGCAGGCGACTTTTCCA
GAGTCTCAAGGGGAGCGTACGCGGGGCAGGTCATGGCTTCAGTTTTTTTCACGATAGTACTGATC
TGCGTCGAGGCCTCACTAGTGCTGCAGGTTCAGGTAGTTTGTAGCAATTTGAGAAGACAATACAG
GATTCTTCTGTTAGGCGCTAGTACGCTGGCCGCCTTAGTACCGATAGGCGTAAGGCTGACTTATT
CAGTTCTAAATTGCATGGTCATCATGCACGCAGGGACTATGGACCACTTGGACTGGTTGGAGTCA
GCAACTAACATCGTGACGACAGTAAGCATATGCTTTTTTTGCGCCGTGTTCGTAGTCAAACTAGG
TTTGGCAATAAAGATGAGAAAGAGGTTGGGGGTCAAGCAGTTTGGCCCAATGAGGGTCATCTTCA
TTATGGGGTGTCAAACTATGACTATACCGGCCATCTTTGCGATATGTCAATATTTTAGCAAAATA
CCAGAGTTTAGCCATAATGTGCTGACCCTTGTAATAATTTCACTACCGTTAAGCTCCATTTGGGC
GGGCTTTGCCCTAGATCAGGCAAATTCTACGGCAAGGAGCAGCGAGTCACGTCACCATTTATGGA
ACATTCTTAGTTCTGACGGAGCGACAAGGGATAAGCCATCTCAATGTGTATCATCTCCCATGACC
AGTCCAAACACAACCTGCTATAGCGAGCAATCAACGAGTAAGCCGCAGCAAGATCCAGAGAACGA
GTTTGGCATCAGCGTGGCGCATGATATTAGCATCCATTCCTTCAGAAAGGATGCACACGGACACT
TG
A. nidulans; AnidSte2
ATGGCTACCCACAACCAAATCTCTGATCAATGTCAATGGTCTTACCCAGAAGTCTTCACCACTCA
AGCTGTCGAAGAACCAACCGCCGAACCAGCTTCTTACCACTTGCACTCTACCTTGACTATTATGG
CTTCTAACTTCGACCCATGGAACCAAACCATTACCTTCAGATTGGAAGACGGTACTCCATTCGAC
ATTTCTGTCGACTACTTGGACGGTATCTTGCAATACTCTATCAGAGCTTGTGTCAACTACGCTGC
TCAATTGGGTGCTTCTGTCATTTTGTTTGTTATCTTGGTCTTGTTGACTAGAGCCGAAAAAAGAG
CTTCTTGTTTGTTCTGGTTAAACTCCTTAGCTTTGTTGTTGAACTTCGCCAGATTGTTGTGTGAC
GTCTTGTTCTTCACCGGTAACTTCGTCAGAATTTACACTTTGATCTCCGCTGACGAATCTAGAGT
TACTGCTTCCGACTTGGCTACTTCCATCGTCGGTGCTATCATGACCGCTTTGTTGTTGACCACTA
TTGAAATTTCTTTGGTTTTGCAAGTCCAAGTCGTTTGTTCTAACTTGAGAAGAATCTACAGAAGA
GCCTTGTTGTGTGTTTCCGCCGTCGTTGCCACTGCTACCATTGCTATTAGATACTCCTTGTTGGC
TGTCAACATTAGAGCTATTTTGGAATTCTCCGACCCAACTACTTACAACTGGTTGGAATCTTTAG
CTACCGTCGCCTTGACCATCTCCATCTGTTACTTCTGTGTCATCTTCGTCACCAAGTTAGGTTTC
GCTATTAGATTGAGAAGAAAGTTGGGTTTATCTGAATTGGGTCCAATGAAGGTCGTCTTCATCAT
GGGTTGTCAAACCTTGGTCATCCCAGGTAAAAGAACCTTGTCTTCTTTGATTCCACCAGTCATTG
TTTCTATTACTCACTACGTCTCCGACGTCCCAGAATTGCAAACTAACGTTTTGACTATCGTCGCC
TTGTCCTTGCCATTGTCCTCTATTTGGGCTGGTACCACCATTGACAAGCCAGTCACTCACTCTAA
CGTTAGAAACTTGTGGCAAATCTTGTCCTTCTCTGGTTACAGACCAAAGCAATCTACCTACATTG
CTACCACTACTACCGCTACTACCAACGCTAAGCAATGTACCCACTGTTACTCTGAATCTAGATTG
TTGACTGAAAAGGAATCTGGTCGTAACAACGACACTTCTTCTAAGTCTTCCTCCCAATACGGTAT
CGCTGTCGAACACGATATTTCCGTTAGATCTGCTCGTCGTGAATCTTTTGACGTC
A. niger; AnigSte2
ATGGCATCAACGGACCCGTTCGCTCAAAATGTAACCTTCCACACTGGAGACGGAACCCCATTTGA
AGTTAACGTACAAGAAATTGACACTTTTCTACAATATTGTATCCAAATTTGTACCAATTACGGGG
CACAACTAGGAGCATCTATAGTTTTATTTGTAATATTATTACTTTTAACGAGGCCTGAAAAAAGA
TCTAGTAGTGTGTTTATCCTGAATTGCGCGGCCTTGCTTTCTAATGTAGGGCGTCTATTCTGCCA
AATACTATACTTCACCACCGATTTTGTAAGGGTATATGCATACTTCTCCGGGGATTTTTCAAGGG
TGCCGACTTCCGCCTATGCTAACTCCGTAATAGCTGTGGTCTTTTATACGATTTTAGTAATCTGT
ATTGAAGCAAGCTTGGTTTTACAAGTTCAGGTAATCTGCGCCAATCTGGGTAGGGTCTATAGACA
GGTCTTATTAAGTCTATCAATTGTTGTTGCCCTAATACCCGTGGGTTTCAGAATGGCCATGATGG
TAGAGAATGCCATAGCCATCATGGCGGCAGAGTCTTCCATTCCACTAATATGGCTGCAAAGCGCC
GAGAATATTACGGCCACCATATCAATTTGCTTCTTTTGCGCCGTTTTTGTGGCCAAATTGGGATT
TGCGATAAGACAGAGAAGAAGGCTTGGCGTCCGTGATTTCGGGCCTATGAAAGTTATATTTATTA
TGGGCTGTCAGACATTAGTCATCCCAGCGACGTTTAGTATAATACACTATATAGCCGCCGTCCCA
GCTTTATCCTCTAATGTAGCGACCTTGGTCACGATTTCACTGCCATTGAGTAGTATTTGGGCAGG
CGTGGCGCTGACAAAGGTCAGCTCCGGTGCGGGGACCGGAAGCGGTACTGGGAGCGGCTCCAGTG
GAAGGAACTTGTGGAACGTGCTGAGCTTTCCCGTATGGGATAGTAGAAATACCACCAGTACCACA
ACGTCAAGTTCCACTCCAACCGTGAATCCAGTGAGCACCTGTTATGCAGATAACGGGAGTTCTAA
ACAATGCAAGCAACAAACGAACACTGAGGAGATTGGGATAACAGTCGAACATGAGATTTCTGTTC
AAAGTTTCAAGAGGGAGAAGGACATGGTG
A. novofumigatus; AnoSte 2
ATGAATAGCACATTCGACCCTTGGACACAAAATATTACGCTGACGCAGAGTGACGGTACTACAGT
GATATCCTCCCTGGCACTTGCTGATGACTATCTACATTACATGATTCGTCTGTCAATTAACTACG
GAGCACAACTAGGGGCGTGTGCCGTCTTGTTATTGGTACTATTACTGTTGACCCGTCCGGAGAAA
CGTATCAGTAGCGTTTTTGTGCTGAACGTGACCGCTTTACTGGCAAATATAATCAGACTGGGATG
TCAATTGTCATATTTCAGCACGGGGTTTGCTCGTATGTACGCACTGCTGGCGGGGGACTTCTCAA
GGGTATCCAGAGGAGCCTACGCTGGTCAGGTTATGGCCAGTGTAAGTTTCACAATTGTTCTGATA
TGTGTCGAAGCAAGCTTAGTACTGCAAGTTCAGGTGGTATGTAGTAATCTTAGACGTAGGTATAG
GATATTGCTGCTTGGTGCAAGCACCCTTGCCGCACTGGTTCCAATTGGGGTGAGGCTAACTTACA
GCGTTCTTAATTGTATGGTAATCATGCATGCAGGAACTATGGATCACTTGGATTGGTTGGAGTCC
GCTACCAACATTGTAACTACCGTCTCCATTTGTTTCTTCTGCGCTGTATTTGTCATTAAGCTGGG
CCTGGCCATTAAAATGAGGAAGAGATTAGGTGTTAAACAATTTGGTCCGATGAGGGTTATATTCA
TTATGGGATGTCAAACAATGACTATCCCCGCCATATTCGCCATTTGTCAGTATTTTTCCAGAATC
CCAGAGTTTAGTCATAATGTTTTAACCCTGGTCATCATCTCCCTTCCGCTATCTTCCATTTGGGC
AGGATTCGCATTAGACCAGGCAAATAGTACGGCAAGATCTACGGAATCCAGACACCATCTATGGA
ACATTCTTAGCAGTGACGGCGCGACTAGAGATAAGCCCACTCAATGTGTGTCCTCACCGATGACT
TCCCCCAACACCACGTGCTACAGCGAGCAAAGCACAAGTAAGCCCCAACAAGATCCCGAGAACGG
CTTTGGCATTAGTGTGGCTCATGATATATCCATCCACAGTTTTAGGAAAGGCGCGAACGGTATAT
TCGAGCAATCCCTACACCTAAGAAATGCT
A. terreus; AteSte2
ATGGGCAGTACTTTCGATCCATTCTCCCAGTCCATCACTCTTAGGGGTCCCGCTGAAAACGAGAT
CACGATACCGATCGAAACCGTTGACTTCTTTTACCAGTATTGCATTAGAATTTGCATAAACTACG
GTGCTCAGTTAGGTGCAAGCATCGTTTTATTTATCATACTACTGTTGTTGACCAGGCCCGAAAAG
AGGCGTTCTTCCGTATTTGTACTGAATATTACGGCGCTTCTATTGAATGTTTCTAGACTTATCTG
CCAATTGATCTATTTTACTTCTGAATTTGTCCGTTTATACGCGTATTTCGGCAACGATTTTTCTA
GAGTGCCCCCGTCCGCGTACGCCGATTCAATATTGGGGGTCGTTCTTGTCACTCTACTTATTGTT
TGCATCGAAGCGTCATTAGTCCTACAGGCACAAGTAGTTTGTGCTAATCTAAGAAGGGGCTACCG
TAGGATTCTTTTAGCTGTTTCAATAGTGATGGCTTTGGTCCCGATAATATTCAGGTTTGTGTACA
TGGTGCAAAATTGTTGGCATATAATGCATGCAGCAGAGACAATTAGACTGAATTGGTTAGAATCA
GCCACAAATGTAGTCATCACGATATCTATATGTTTTTTCTCCGCAGTTTTCATAACTAAGCTTGG
ATTCGCGATGCATTTAAGAAAGAGACTGGGCATGACCGATTTCGGTCCTATGAAAGTAATCTTTG
TGATGGGCTGCCAGACGATGGCTATCCCAGCTATCTTTAGTATATTGCAGTACATCGTAAGCGTA
CCCGAACTGGCATCAAATATATTTAGCTTAGTGACTATCTCCCTGCCCCTATCAAGCATCTGGGC
AGGCTTCACGATTGACCAAAGAGCACGTGATTCAGGTATGTCAAGCTCCAGGAACAACTTATTTC
AGGCGTTAAGCTTCTCTAAAGAAGCAAAGACTAGGCAAAGTAGCACCGTCACATATGCTTCCCAA
CCGAGTTCCAAGAGTCACACGCTTTGTTATGCGGAACTTTCTCCCGCTAAGCAACAATCTGACGC
AGAAGCGGCCTACGGGATCGCAGTCGAAAGAGACATCAGCATACATTCTTGTAGGAAAGACGGAA
AGGCCCCGAATGTG
A. udagawae; AuSte2
ATGAATTCCACGTTCGATCCCTGGACACAGAACATTACCCTGACGCGTTCTGACGGGACCACTGT
CATTAGTAGCTTGGCACTGGCTGATGAGTTCCTGAGATATATGATCCGTTTGAGTATAAATTACG
GAGCGCAGCTGGGAGCTTCAGTTGTACTGCTTTTGGTACTGATGCTGCTGACGCGTCCTGAAAAG
CGTGTCAGTTCCGTCTTCATGCTAAATGTTGCTGCGCTGCTGGCTAATATCATCCGTTTGGGTTG
TCAACTGAGTTATTTTAGTACAGGCTTCGCGAAGATGTATGCATTACTGGCCGGAGACTTTAGTC
GTGTTAGTAGAGGGGCGTACGCGGGTCAAGTGATGGCCTCAGTATTCTTTACGCTTGTTTTAATT
TGTGTCGAAGTAAGTCTGGTTTTGCAGGTCCAAGTCGTCTGCAGCAATTTACGTCGTCAGTACAG
GATACTTCTATTGACCGCTAGTACCTTTGCTGCATTAGTGCCTATAGCTGTCAGGCTGACGTACT
CCGTACAAAACTGCATGGTGATAATGCATGCCGGCACGATGGACCATCTAGATTGGCTAGAATCA
GCTACTAACATCGTCACCACTGTCTCTATCTGCTTCTTTTGTGCAGTTTTCGTCGTGAAATTGGG
CCTTGCGATCAAGATGAGAAAGAGGTTGGGCGTAAAACAGTTTGGGCCGATGAGAGTCATATTTA
TCATGGGATGCCAGACGATGACAATACCCGCTATTTTTGCAATTTGTCAGTACTTTAGTAAGATC
CCAGAGTTCTCCCATAATGTTCTGACCCTTGTAATAATCAGCCTTCCTCTATCTTCAATTTGGGC
GGGCTTCGCTCTGGAGCAGGCCAATCCTACCGCCAGAAGTACAGAGAGCCGTCATAATCTTTGGA
ATGTATTGTCCTCAGATGGAGCTGCGAGAGACAAACCTTCCCAATGTATTTCCTCCTCATTGACA
AGCCCGAACGCAACATGCTATTCAGAACAAAGTACGTCAAAATCTGAACAAGATCCTGCAAATAG
GTTCGGGATTAGCGTCGCCCACGACATATCCATCCGTAGTTATCAGAAGGATGCACACGGCGATG
TA
A. viridinutans; AvSte2
ATGAATTCCGCATTCGACCCCTGGACTCAGAACATTACTTTAACACGTTCCGACGGTACAACCGT
TATTTCTTCTTTAGCACTAGCTGACGAATTTTTGCGTTACATGATTAGATTGAGTATAAACTATG
GAGCACAGCTTGGGGCGTGTGTCGTGTTACTGCTGGTTCTGCTATTGCTAACTAGACCCGAAAAA
AGAGTGTCCTCCGTTTTCGTCCTGAATGTGGCGGCGCTGCTTGCCAACATAATCCGTCTAGGCTG
TCAACTTAGTTATTTCACGACCGGCTTTGCTCGTATCTATGCGTTGTTAGCCGGCGACTTCTCTC
GTGTGTCTCGTGGCGCCTACGCCGGCCAGGTTATGGCTAGTGTATTCTTCACACTAGTCCTTATC
TGCGTTGAAGTAAGTCTAGTTTTGCAAGTACAGGTTGTCTGCTCCAATCTGAGAAGGCAATACAG
AATCTTGTTATTGGGCGCAAGTACACTAGCCGCATTGGTTCCAATCGCTGTGAGGCTAACATACT
CCGTCCAGAATTGCATGGTCATAATGCACGCCGGAACGATGGATCATCTAGACTGGTTAGAGTCT
GCTACCAATATCGTAACCACGGTTTCTGTATGTTTCTTTTGCGCAGTGTTCGTGGTCAAGTTAGG
GCTGGCGATTAAGCTGCGTAAAAGGCTTGGTGTAAAACAATTTGGTCCAATGCGTGTAATCTTCA
TTATGGGTTGTCAAACTATGACAATCCCAGCTATCTTTGCCATTTGTCAGTACTTTTCTGAGATC
CCAGAGTTCTCCCACAACGTATTGACATTAGTCATAATCAGTTTGCCCCTAAGTTCCATTTGGGC
TGGCTTCACTCTTGAGCAGGCTAATTCCACGGCTAGATCTACAGAAAGCAGACATAACCTTTGGA
ACATACTTAGCTCCGATGGTGCTGTCAGGGATAAACCGGGACAATGTATTAGTAGCCCACTGACG
AGTCCTAACACCACGTGTTACTCCCAACACTCTACAGGGAAACCTCAGCAAGATCCGGAAAACGG
TTTCGGTATTGCGGTTGCCCACGATATCAGTATCCACAGCTATCAAAAGGATGCGAGGGGTGATG
TA
Aspergillus pheromone precursors: Annotated
Aspergillus pheromone precursors. Underlined:
inferred mature peptide sequence.
A. clavatus; accession number: A1CE26 [217]
MKLLSVVLATLAATTVQAGAMAQWCELPGQGCYMIKRAAEASGEVKRSASAVADAVAEAFPDAAL
WCELPGQGCHKVKRAAEAAEEVKRSADAFAEAMAALEHLE
A. fischeri; accession number: A1DM33 [217]
MRLLSLVLATFAATAVQADITPWCHLPGQGCYMLKRAADASDEVRRSASAVAEAVAEAFPQTPWC
HLPGQGCAKAKRAAEAAEEVKRSADAFAEAMAAFEKE
A. flavus; accession number: B8N2L7 [217]
MKLISVVVAALAATSVQAGVLQKWCSLPAQGCYMLKRAADASGDVRRSAEALAEAMPDAEALAKW
CALPGQGCLKAKRAAEAVEEARRSADALADAMADLGEY
A. oryzae; accession number: A0A1S9DPJ9
MKLISVVVAALAATSVQAGVLQKWCSLPAQGCYMLKRAADASGDVRRSAEALSEAMPDAEALAKW
CALPGQGCLKAKRAAEAVEEARRSADALADAMADLGESLPRALSF
A. fumigatus; accession number: Q4WNH4 [217]
MKLLSLVLASFAAAAVQAHITPWCHLPGQGCYMLKRAADASDEVRRSASAVAEAVAEAFPQSPWC
HLPGQGCAKAKRAAEAAEEVKRSADAFAEAMAAFEKE
A. lentulus; accession number: A0A0S7DHY4
MKLLSLVLATFAATAVQADITPWCHLPGQGCYMLKRAADASDEVRRSASAVAEAIAEAFPQTPWC
HLPGQGCAKAKRAAEAAEEVKRSADAFAEAMAAFEKE
A. nidulans; accession number: Q7S174 [217]
MKLFFVSILLAALLATAVKAAPAAELQHRWCRFAGRICPPTKRTADALNFVKREAEAVAEPFKIN
RWCRFRGQVCGKAKRAAEAIGNVKLSAEAVADAMAFLDELTREEYAQLAKDFGHLKESDNSDG
A. niger; accession number: A2RBH8 [217]
MKLVSLVLAALAATTVQAAALQRWCVLPGQPCNMIKRAADASGEVKRSADALAEAIADATPETLQ
RWCVLPGQPCNKIKRAVEAGSEVKRSADAFAEAMADIDESDFQ]
A. novofumigatus; accession number: A0A211CDQ5
MKLLSLVLATFAATAVQADITPWCHLPGQGCYMLKRAADASDEVRRSASAVAEAVAEAFPQTPWC
HLPGQGCAKAKRAAEAAEEVKRSADAFAEAMAAFEKE
A. terreus; accession number: Q0CFX7 [217]
MKLITLVLATLAATSSVQAAKMHKWCWLPGQGCYMLKRAADVTGEVKRHAEALAEAMPDTSPDGI
HKWCWLPGQGCLKVKRAADAVDEVKRSADALAEAMAAIEELHAEDLVEIAIGDKEDAAMCDSDYV
LSFDIR
A. udagawae; accession number: A0A0K8LJ87
MKLLSLVLATLAATAVQADITPWCHLPGQGCYMLKRAADASDEVRRSASAVAEAVAEAFPQTPWC
HLPGQGCAKAKRAAEAAEEVKRSADAFANAMAAFEEE
A. viridinutans; accession number: XP 043129994
MKLLSLVLATLAATAVQANVTPWCHLPGOGCYMLKRAADASDEVRRSASAVAEAVAEAFPQTPWC
HLPGQGCAKAKRAAEAAEEVKRSADAFADSMAAFEKE

### Results

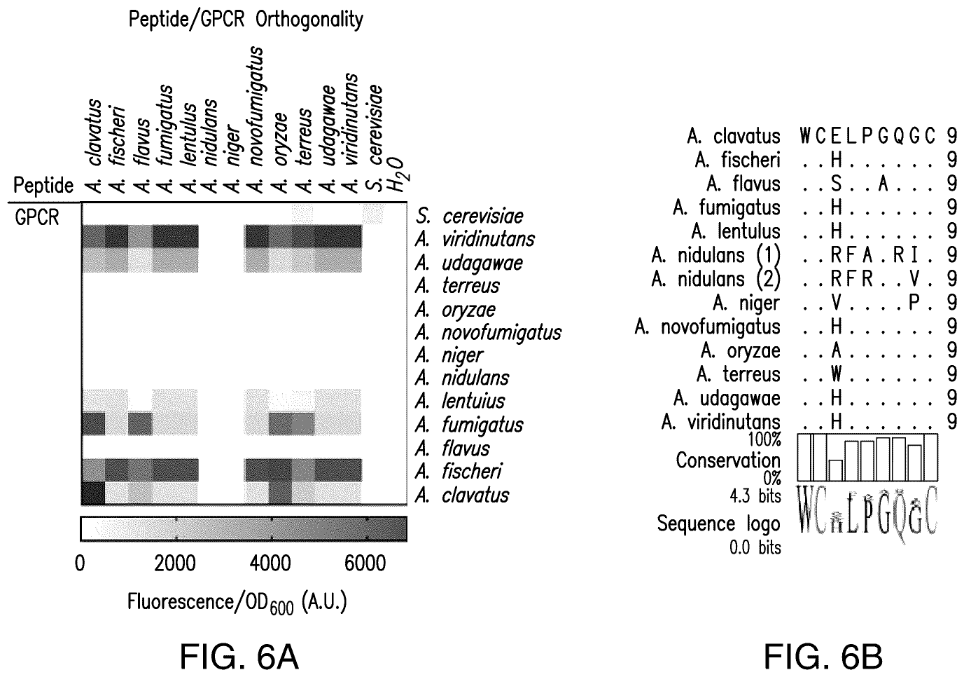

Based on clinical and agricultural relevance, GPCRs and pheromone peptides were selected from 11 Aspergillus species: clavatus, fischeri, flavus, lentulus, nidulans, niger, novofumigatus, oryzae, terreus, udagawae and viridinutans. GPCR ORFs were codon-optimized and cloned into constitutive GPCR expression cassette plasmids for Golden Gate assembly and yeast integration. Pheromone peptide sequences were assumed and ordered as a tandem of two putative secreted pheromone repeats within pheromone precursor ORF. For A. nidulans pheromone, the first and second peptide repeat within pheromone precursor ORF differ in two amino acids in the 5th and 8th position from N-terminus, so it was assumed the tandem to be the fusion of the first and second non-equal pheromone repeat in the order as they appear within the ORF (FIG. 6B).

Next, 13 GPCRs of all genome-mined Aspergillus species, including A. fumigatus from Example 1 and S. cerevisiae, along with inducible fluorescent readout were integrated into optimized parent strain. S. cerevisiae was included to test cross-reactivity with S. cerevisiae GPCR and peptide, since this peptide/GPCR pair will be used for building multi-cellular amplification communities. Then, each strain was activated with all Aspergillus peptides and S. cerevisiae peptide (FIG. 6A). Most of the Aspergillus GPCRs were responsive to at least one peptide, except A. terreus, A. flavus/A. oryzae (same GPCR sequence), A. niger and A. nidulans which were not responsive to any tested peptide. The most promiscuous GPCR is A. viridinutans with the strongest response throughout all active peptides and the most selective GPCR is A. clavatus with the strongest response with only its cognate A. clavatus peptide and A. oryzae peptide, however, its response is not negligible with other active peptides. A. novofumigatus GPCR showed the weakest response among responsive GPCRs. The majority of peptides have the same sequence (A. fischeri, A. fumigatus, A. lentulus, A. novofumigatus, A. udagawae and A. viridinutans) and the other active peptides differ only in the third amino acid position from N-terminus (A. clavatus, A. flavus and A. oryzae). A. nidulans and A. niger peptide did not activate any tested GPCR. Interestingly, S. cerevisiae GPCR cross-reacted with A. terreus peptide which is the only Aspergillus peptide with conserved tryptophan in the third position just like in the S. cerevisiae peptide (FIG. 6B). With these results it was concluded Aspergillus biosensors cross-reactivity and continued with further sensitivity optimization of the A. fumigatus biosensor.

### Example 5: Biosensor Sensitivity Improvement Via Biosensor Consortia Engineering

To further improve the sensitivity of the biosensor, peptide/GPCR communication language was employed to build signal amplification communities (Billerbeck et al. A scalable peptide-GPCR language for engineering multicellular communication. Nat. Commun. 9, (2018)). As previously demonstrated (Shaw et al. Engineering a Model Cell for Rational Tuning of GPCR Signaling. Cell 177, 1-15 (2019)), a simple two-membered amplification system, where the first member senses a molecule of interest which activates the secretion of S. cerevisiae alpha-factor and the second member senses a secreted alpha-factor which activates the readout, it is possible to achieve around a 1,000-fold decrease in EC₅₀ value. For the A. fumigatus biosensor, different and more complex communication topologies were employed in order to achieve both improved sensitivity and retain robust readout in all members of the community.

First, a one-membered positive feedback loop, where a single strain senses AfuPep and expresses both mCherry and more of AfuPep (yTC523) was tested. Unfortunately, even though the basal expression is very low, there is more than enough of secreted AfuPep to make such strain self-activated. The initial fluorescence intensity is ˜2,500 A.U. and after incubation with 40 μM AfuPep, the fluorescence intensity is only two-fold greater than the background fluorescence. In order to prevent self-activation, a negative regulator is needed that would be homologous to S. cerevisiae alpha-factor protease (Bar1) which would degrade minute amounts of the basally secreted AfuPep, but upon external activation by AfuPep the degradation rate would be overcome.

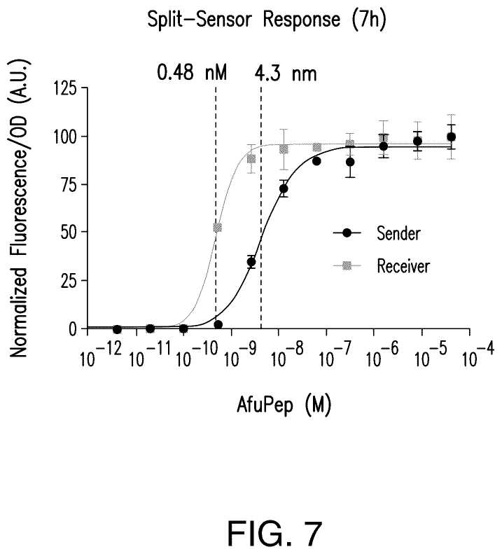

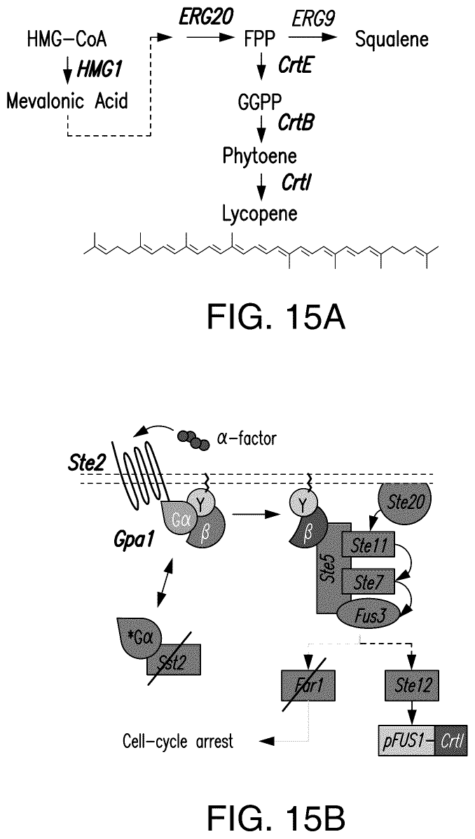

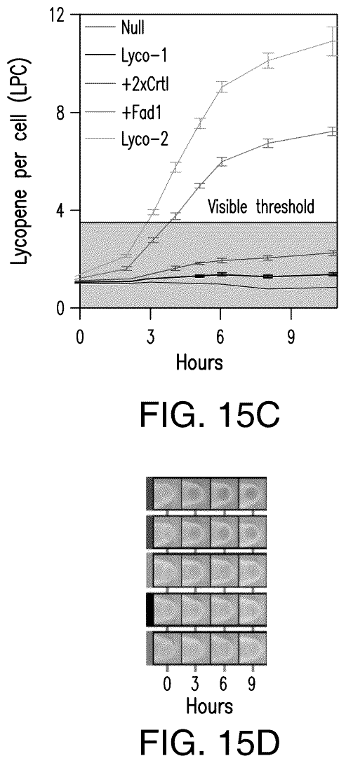

Next, two-membered communities were tested. A first two-membered community where the sender strain uses Ste2 homolog from Aspergillus fumigatus (AfuSte2) to detect cognate pheromone peptide (AfuPep) and conditionally express both α-pheromone and GFP. Complementarily, the receiver strain senses α-pheromone secreted by the sender strain and conditionally expresses mCherry. Two different fluorescent proteins for the sender and the receiver strain were used to individually monitor the members. The direct activation of the sender strain achieves an EC₅₀ of ˜4 nM based on the dose-response curve generated for sender strain's GFP intensity, while the receiver strain in the same system shows a response at 8-fold lower AfuPep concentration (EC₅₀˜0.5 nM) based on the dose-response curve generated for the receiver's strain mCherry intensity (FIG. 7). It is envisioned that the AfuPep and AfuSte2, e.g., of the sender cell, can be switched with the coronavirus epitope and developed cognate GPCR respectively to drive the receiver strain readout expression. For this biosensor, an improved lycopene reporter is used as a readout instead of a fluorescent protein in the receiver strain. Expected limit of detection (LoD) of the final working lycopene biosensor will be at concentrations lower than measured EC₅₀ values from dose-response curves (FIG. 15D).

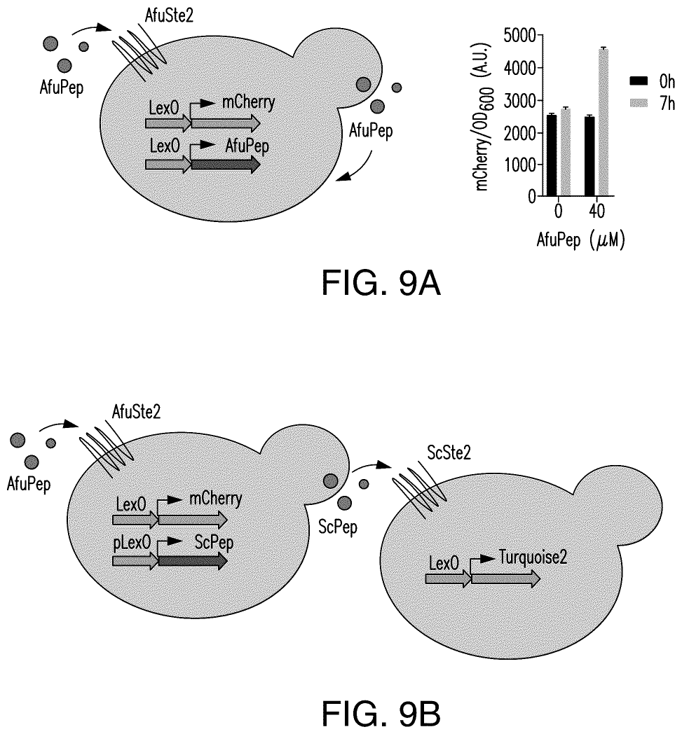

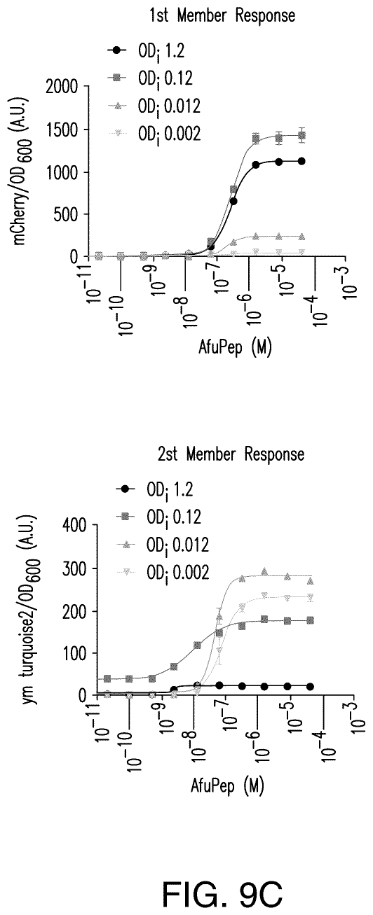

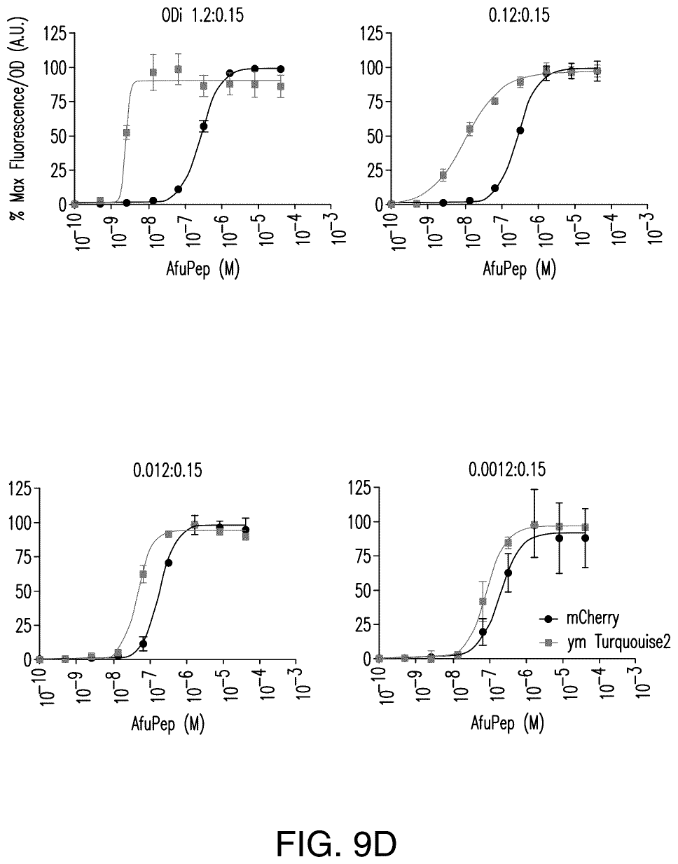

An additional two-membered community, where the first member senses AfuPep and expresses both mCherry and secretes S. cerevisiae pheromone peptide (ScPep) and the second member senses ScPep (ScSte2) and expresses fluorescent protein ymTurquoise2 (FIG. 9B). Each member expresses different fluorescent protein in order to individually track their activation. A starting OD (ODi) of the second member fixed at 0.15 was used and varied ODi of the first member with 1.2 as the highest and 0.002 as the lowest ODi (FIG. 9C). The improvement in sensitivity is expected as an apparent shift in EC₅₀ of the second member (ymTurquoise2 fluorescence curve) to lower concentrations of the AfuPep activating the first member. The ODi of the first member did not affect its EC₅₀, but it did determine the final fluorescence intensity normalized to total OD of the community which dropped with decreasing ODi. The response of the second member showed the strongest overall intensity at lower ODi. In order to monitor the improvement in biosensor's sensitivity, the shift in EC₅₀ was monitored with normalized fluorescence intensities (FIG. 9D). mCherry and ymTurquoise2 have arbitrary intensity units, so their activation was normalized based on % of the maximal fluorescence value for each fluorescent protein. The strongest shift in EC₅₀ was achieved with first member ODi 1.2 with almost 100-fold lower AfuPep concentration and the lowest shift of around two-fold was achieved at the lowest first member ODi 0.002. When determining the amplification community ratio, the strong EC₅₀ shift which comes at high first member ratio and overall community readout intensity which comes with more balanced ratio of each member was leveraged.

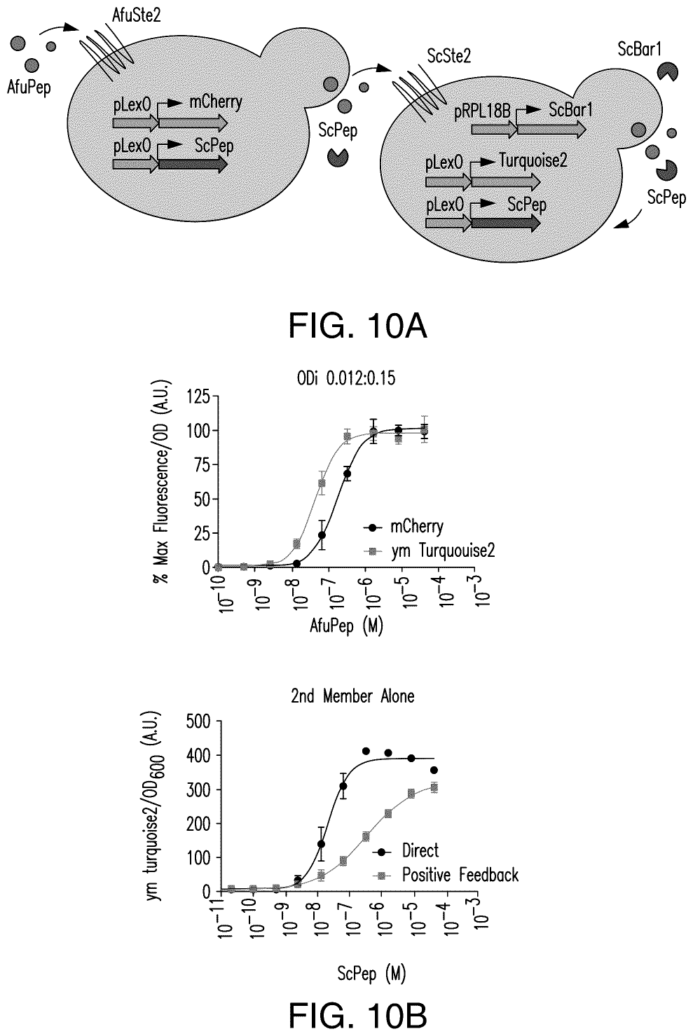

Next, in order to further improve EC₅₀ shift in the two-membered split biosensor community, another topology of two-membered amplification community where the second member additionally secretes ScPep and forms a positive feedback loop was validated (FIG. 10A). In order to prevent self-activation of the second member from basally secreted ScPep, the second member also constitutively expresses protease ScBar1 under intermediate pRPL18B promoter which cleaves any basally secreted ScPep. This community did not lead to improved shift in EC₅₀ compared to simple split biosensor community in FIG. 9B-D with the comparable EC₅₀ shift at Odi 0.0012:0.15-member ratio. It seems the reason for no improvement in sensitivity is that the ScBar1 cleavage of secreted ScPep is too strong and is attenuating the overall response in the second member. To test that, the second members from both split biosensor in FIG. 9B and split biosensor with positive feedback in FIG. 10A were activated alone with their cognate ScPep and compared their dose-response curves. The second member with positive feedback loop showed higher EC₅₀ due to too strong ScBar1 cleavage which overcomes the amplification effect of inducible ScPep secretion (FIG. 10B) and in fact shows worse response compared to a direct ScPep biosensor without positive feedback loop. Building positive feedback loop in the second member with protease ScBar1 at low expression level with pRAD27 promoter led to auto-activation (data not shown). In order to further troubleshoot the ineffective positive feedback loops, other means were investigated to prevent overall signal attenuation by the protease.

In order to implement effective positive feedback loop which would show stronger response compared to direct biosensor, it was hypothesized that the stain could have another close-cognate peptide which would trigger the positive feedback loop strain, but this peptide would not be prone to protease cleavage. With this setting, the externally added non-cleavable peptide would behave like a regular cognate peptide in a direct split biosensor without the protease, since the protease will not affect its concentration and binding to the receptor. The positive feedback loops should still secrete the cognate peptide which is cleavable by the protease in order to prevent the self-activation. As elaborated in Example 7, such close-cognate peptides for CaSte2 GPCR which are not cleaved by CaPep cognate protease CaBar1 were identified and that they activate the GPCR as strongly as the native CaPep.

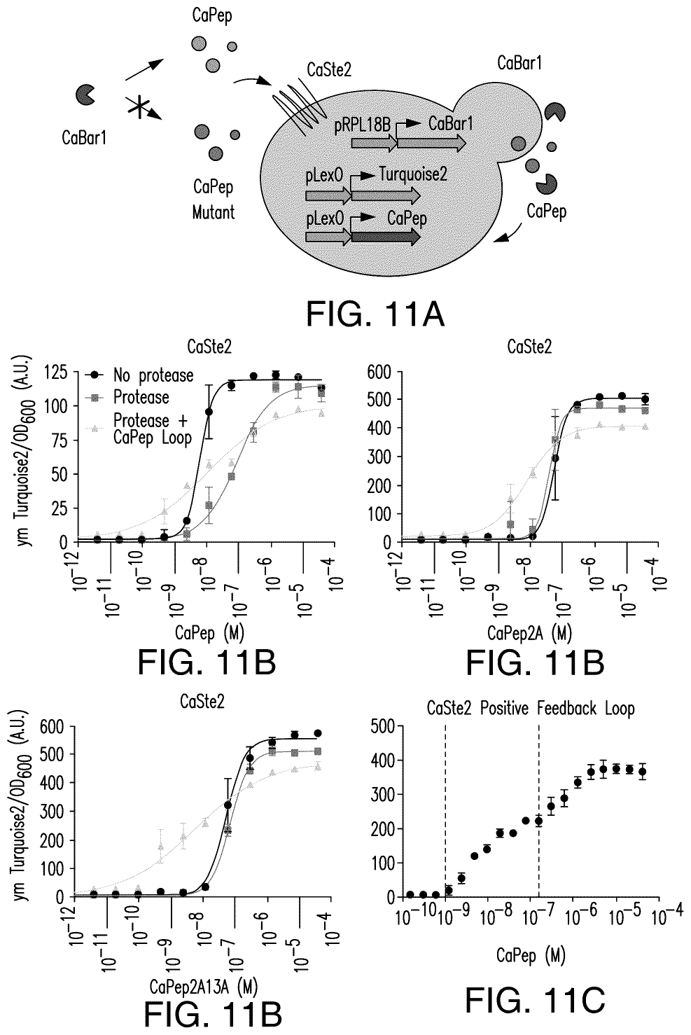

In order to test the hypothesis that non-cleavable activating peptide would improve the positive feedback loop response compared to direct biosensor without the loop and the protease, C. albicans peptide/GPCR/protease pairs were selected and validated in Example 7. Cognate CaPep was selected as a peptide cleavable by the protease CaBar1 and integrated its inducible secretion as part of the positive feedback loop. Then, non-cleavable CaPep mutants CaPep2A (GARLTNFGYFEPG) and CaPep2A13A (GARLTNFGYFEPA) were selected, which have one or two point mutations in their sequence, respectively, as externally activating peptides. The protease CaBar1 was expressed under intermediate pRPL18B promoter (FIG. 11A). The dose-response activation of three different CaSte2 biosensors expressing ymTurquoise2 (without the protease, with protease, or with protease and inducible CaPep secretion) were compared with all three peptides (CaPep, CaPep2A and CaPep2A13A) (FIG. 11B). The activation of biosensors with native cognate CaPep showed EC₅₀ of ˜7 nM in the biosensor with no protease and as expected, a shift in EC₅₀ towards higher concentration in biosensor with the protease (˜90 nM). In biosensor with positive feedback loop, the EC₅₀ goes from 90 nM from the biosensor with only protease back to ˜11 nM, which is similar response as a direct biosensor without the protease. Even though the positive feedback loop did improve EC₅₀ to lower peptide concentration compared to biosensor with protease, the response is still very similar to direct biosensor, so there is not much gained benefit from implementing a positive feedback loop, compared to simple direct sensor. Next, the biosensing strains were activated with the two non-cleavable mutants CaPep2A and CaPep2A13A. Both peptide mutants did not show significant shift in EC₅₀ to higher concentration in biosensor strain with the protease compared to strain without the protease (for CaPep2A from ˜50 nM to ˜40 nM and for CaPep2A13A from ˜50 nM to ˜70 nM).

However, when mutant peptides activated the strain with positive feedback loop, EC₅₀ lowered by one order of magnitude (˜7 nM) compared to the response of strain with no protease. By activating the positive feedback loop with non-cleavable peptide, improvement in C. albicans biosensor sensitivity was achieved compared to direct biosensor without the protease. Furthermore, upon closer inspection of the C. albicans positive feedback loop dose-response, it is evident that the four-parameter Hill function does not fit well to the response:concentration points compared to fit in direct and only protease biosensors (FIG. 11B). There seemed to be a trend in positive feedback loop being activated with all three peptides (CaPep, CaPep2A, CaPep2A13A) where the response:concentration points follow a curve with two inflection points.

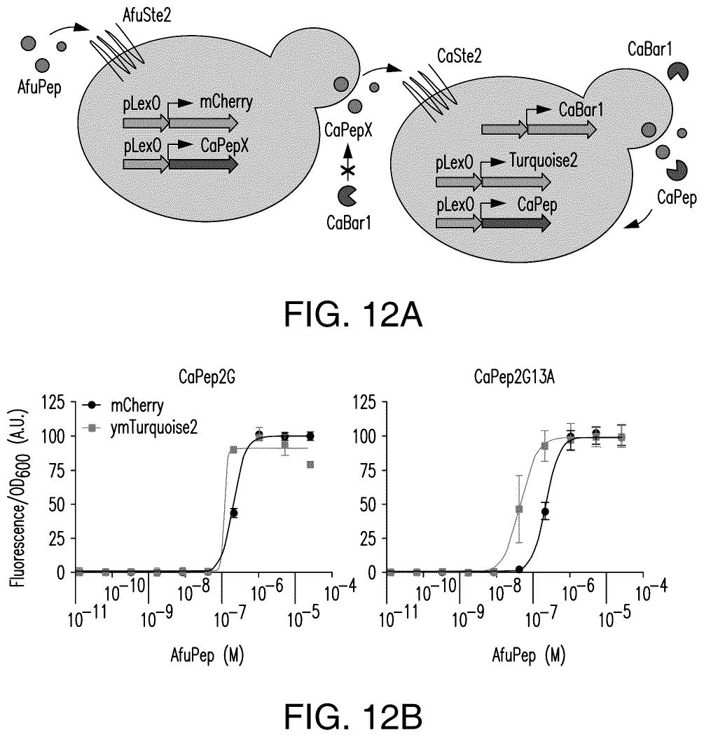

To get a better sense of the dose-response trend, a positive feedback loop was activated with cognate CaPep with smaller peptide serial dilutions over the same peptide concentration range as reported in previous graphs (FIG. 11C). Indeed, the dose-response trend truly shows two distinct inflection points and the response can be divided into two response phases. Going from lower peptide concentration to higher, the first response phase starts at ˜1 nM peptide concentration where a fluorescence intensity above zero A.U. was first seen and finishes roughly at 100 nM where the second phase starts and continues all the way to the highest measured CaPep concentration. For such biphasic dose-response, Hill function is not ideal model to estimate response parameters, such as EC₅₀, and some other more complex models should be employed. The hypothesis is that the fluorescence response in the first phase originates primarily from the CaPep inducibly secreted by the yeast, while the fluorescence response in the second phase originates primarily from the externally added CaPep. For simplicity, if each phase was treated as their own separate dose-responses which can be fit in Hill function, the first phase has “EC₅₀” ˜5 nM and the second phase has “EC₅₀” ˜100 nM. If the second phase is primarily activated from the externally added CaPep, then the “EC₅₀” of the second phase would roughly correspond to EC₅₀ of the strain with only protease being activated by externally added CaPep. Indeed, the EC₅₀ for such strain activated by CaPep from FIG. 12B is ˜90 nM. That being said, the positive feedback loop activated with non-cleavable peptide mutants actually shows far better sensitivity with estimated “EC₅₀” from the first phase compared to EC₅₀ of the direct biosensor without the protease. CaPep2A shows one order of magnitude lower “EC₅₀” (˜1 nM) and CaPep2A13A shows two orders of magnitude lower “EC₅₀” down to high picomolar sensitivity (˜0.3 nM) (FIG. 12B). With these promising results, this modified activation of positive feedback loops was implemented into the A. fumigatus two-membered split biosensor.

In order to implement C. albicans positive feedback loop activated with non-cleavable CaPep2A and CaPep2A13A into A. fumigatus two-membered split biosensor, the first member in the A. fumigatus biosensor community was engineered to be activated by AfuPep and to inducibly express mCherry and either of the two non-cleavable peptide mutants (CaPep2A or CaPep2A13A) (FIG. 12A). The second member in the A. fumigatus biosensor community is the C. albicans positive feedback loop strain presented and validated in FIGS. 11A-11B. The constructed inducible CaPep2A and CaPep2A13A secretion components were not functional, as the first member strain did not activate the second member at all (data not shown). It was first hypothesized that maybe the Ste13 processing site EAEA directly upstream from the mutant CaPep sequence in MFalpha prepro region is not being processed correctly in the ER. Previous study by Billerbeck et al. showed that secreted peptide sequence can affect its secretion efficiency and adding Ste13-processing site upstream usually helps with the processing, but for some peptides, EAEA omittance improves the peptide secretion. For mutant CaPep secretion, the removal of Ste13-processing site did not help with the secretion (data not shown). It was thought that the mutation identity within the peptide is fundamentally tampering with the peptide processing, as the original cognate CaPep is efficiently processed and secreted (Billerbeck et al.). The mutated amino acid residue within both used non-cleavable peptides have switched phenylalanine in the second position from N-terminus to alanine.

It was hypothesized that specifically the alanine in the second position in the peptide could be tampering with the peptide processing, since conserved fungal peptide precursor processing sequences usually contain alanine in the second position of the cleaving repeats, such as KA and EA. This would mean that the first two amino acids in the mutant CaPeps could be cleaved in the Golgi apparatus and truncated non-functional peptides end up being secreted. To prevent that, mutant peptide sequences were cloned in the inducible peptide secretion cassettes having glycine instead of alanine in the second position (CaPep2G and CaPep2G13A). This modification seemed to improve mutant CaPep secretion, as activated first member in the A. fumigatus split biosensor did activate the second member in the biosensor community (FIG. 12B). Two-membered A. fumigatus split biosensor with positive feedback loop in the second member activated by CaPep2G secreted by the first member did not improve EC₅₀ in the second member's response. When the split biosensor has CaPep2G13A as the activating peptide of the second member, the EC₅₀ shifts about one order of magnitude towards lower AfuPep concentration. This two-membered A. fumigatus biosensor setup does not improve sensitivity better than the direct split sensor presented and validated in FIGS. 9B-9C. The reason for that might be that the CaPep2G and CaPep2G13A have different dose-response and CaBar1 cleavage efficiency compared to CaPep2A and CaPep2A13A due to sequence differences.

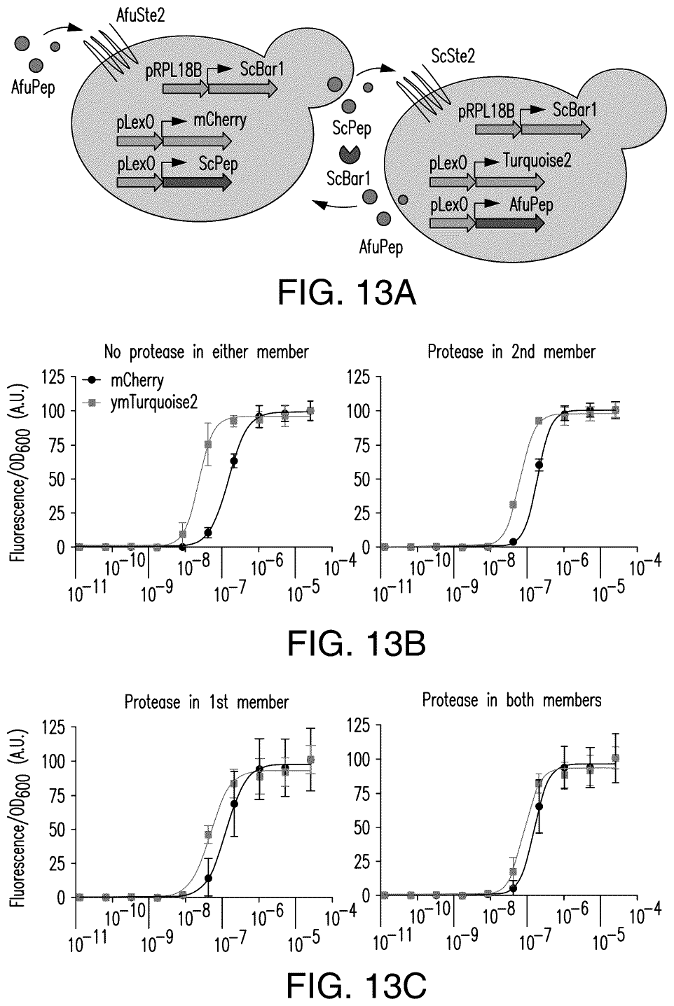

For the final amplification setup, the two-membered positive feedback split biosensor was validated where the first member senses AfuPep and expresses both mCherry and ScPep and the second member senses ScPep and inducibly expresses ymTurquoise2 and AfuPep which further activates the first member. Additionally, in each of the members in this biosensor, low constitutive expression of protease ScBar1 was introduced, as it was anticipated that the two-membered positive feedback community could self-activate (FIG. 13A). The dose-response of these communities were validated having ScBar1 in neither member, in one or the other member, or in both members (FIG. 13B). ScBar1 expressed in both members did not significantly shift EC₅₀ to lower peptide concentration in the second member. ScBar1 expressed in either the first member or the second member shifted EC₅₀ comparably by half an order of magnitude. Ultimately, no expression of protease ScBar1 was necessary, as the two-membered positive feedback loop did not self-activate in the measured settings and such community showed EC₅₀ improvement by one order of magnitude. However, such shift is not significantly better than direct two-membered split biosensor presented and validated in FIGS. 9B-9C at the same member ratio (1:1).

### Example 6: Improvement of Inducible Lycopene Biosynthesis

In order to meet the needs of simple low-tech detection, the fluorescent readouts were replaced in the strains of the single- and multi-communities with a red pigment lycopene readout as previously described (Ostrov et al. A modular yeast biosensor for low-cost point-of-care pathogen detection. Sci. Adv. 3, (2017)). Furthermore, it was aimed additionally investigate lycopene biosynthetic pathway optimization and yeast culturing conditions in order to achieve faster visible lycopene color onset.

Materials and Methods
CrtI gene sequence:
ATGAAGAAAACCGTAGTGATTGGTGCAGGTTTTGGTGGTTTAGCTTTGGC
TATACGTCTACAAGCTGCAGGTATTCCTACAGTGCTATTGGAGCAAAGAG
ACAAACCAGGAGGAAGAGCTTATGTTTGGCACGATCAAGGCTTTACCTTT
GATGCTGGTCCTACAGTCATCACTGATCCTACTGCATTGGAAGCTTTGTT
CACCTTAGCTGGTAGAAGAATGGAAGATTATGTCCGTCTATTGCCTGTCA
AGCCGTTTTACAGATTGTGTTGGGAATCTGGTAAAACCCTAGATTACGCC
AATGACAGTGCTGAACTAGAAGCTCAGATTACGCAGTTTAATCCCAGAGA
TGTCGAAGGTTACAGGAGATTCCTTGCCTATTCCCAAGCTGTTTTCCAAG
AGGGTTATCTTCGTTTGGGTTCAGTTCCATTCCTGTCCTTTAGGGATATG
CTTAGAGCAGGTCCTCAGTTGTTGAAGCTACAAGCATGGCAAAGTGTGTA
TCAGTCTGTTTCGAGATTTATCGAGGATGAACATCTGAGACAAGCATTCT
CATTCCACAGTCTTCTAGTTGGAGGTAATCCCTTTACCACATCGAGCATA
TATACGTTGATTCACGCTTTGGAAAGAGAATGGGGAGTTTGGTTTCCTGA
AGGTGGAACAGGTGCTTTGGTTAATGGTATGGTGAAGCTATTCACGGATT
TGGGTGGAGAAATAGAGCTGAATGCAAGAGTGGAAGAACTTGTTGTAGCA
GACAACAGAGTCTCACAAGTTAGACTTGCTGATGGTAGGATCTTCGATAC
AGATGCTGTAGCTTCAAACGCAGATGTAGTGAACACTTATAAAAAGTTGT
TGGGACATCATCCTGTTGGACAAAAGAGAGCAGCTGCTTTGGAGAGGAAA
TCTATGAGCAACTCGTTGTTTGTCCTTTACTTTGGGCTGAATCAACCACA
CTCACAACTAGCTCATCACACAATCTGCTTTGGTCCTAGATACAGAGAGC
TGATAGATGAAATTTTCACTGGATCTGCTTTAGCAGACGATTTTTCCCTG
TACTTGCATTCACCATGTGTTACTGATCCCTCTTTAGCACCACCTGGTTG
TGCTAGCTTCTATGTACTAGCACCTGTACCACATTTGGGTAATGCTCCAT
TAGATTGGGCACAAGAAGGACCGAAATTGAGGGATAGGATCTTCGACTAT
TTGGAAGAACGTTACATGCCAGGTTTGAGATCTCAGTTGGTTACACAGAG
GATATTCACACCAGCTGATTTTCATGATACTCTAGATGCGCATTTAGGTA
GCGCTTTTTCCATTGAGCCACTTTTGACGCAAAGTGCTTGGTTTAGACCA
CACAACAGAGATTCTGACATTGCCAATCTGTACCTAGTAGGTGCAGGAAC
TCATCCAGGAGCTGGTATTCCTGGAGTTGTAGCTTCTGCTAAAGCTACTG
CTAGTCTGATGATCGAGGATTTGCAGTAA

### Results

First, a parent strain yTC412 analogous to yTC370 was engineered which has integrated all relevant genes in lycopene biosynthetic pathway. This strain is only missing GPCR as a minimal component in order for the strain to become peptide responsive. The GPCR and additional biosensing components can be assembled using Golden Gate assembly and integrated using acceptor integration plasmid.

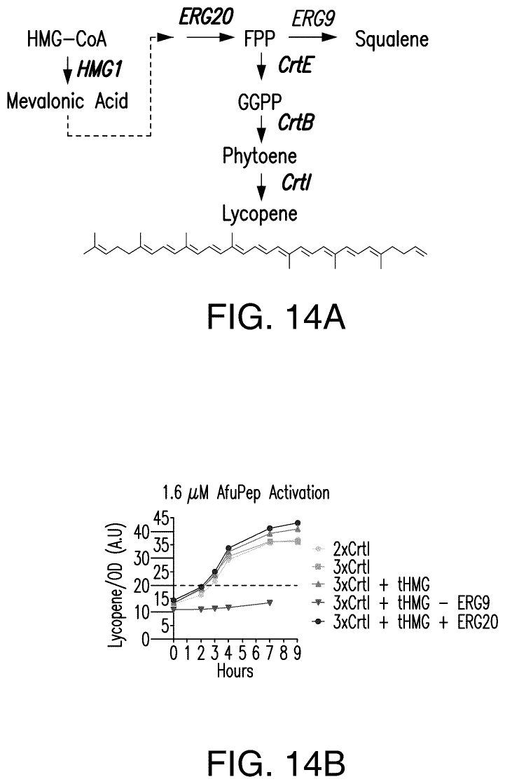

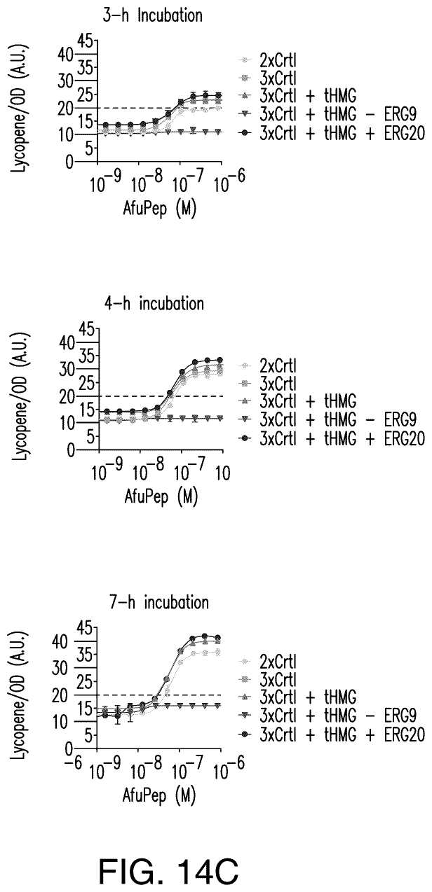

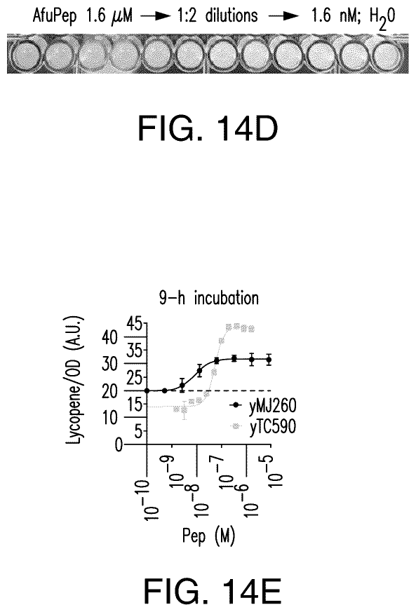

Lycopene is a carotenoid pigment naturally produced by bacteria and plants. Lycopene can be biosynthesized in yeast from native precursor farnesyl pyrophosphate by introducing only three additional genes: geranylgeranyl diphosphate synthase (CrtE), phytoene synthase (CrtB) and lycopene synthase (CrtI) (FIG. 14A; FIG. 15A). In order to engineer lycopene parent strain yTC412, the first two biosynthetic genes CrtE and CrtB under strong constitutive promoters pTEF1 and PGK1, respectively, were integrated into yTC370 (FIG. 15B). To connect lycopene production to peptide activation, two copies of the last pathway gene CrtI (lycopene synthase) were placed under control of peptide-inducible promoter (LexO(6×)-pLEU2m). Additionally, another copy of the endogenous FAD1 (flavin adenine dinucleotide synthase) under constitutive promoter from TEF1 was integrated into the parent strain (FIG. 15C). The enhanced strain v2.0 (Lyco-2) exhibited strong lycopene production, crossing the visible lycopene threshold in only 3 hours (FIG. 15C). These modifications generated a phenotypically white strain that became visibly orange within hours upon exposure to the α-factor, which activated the pheromone response pathway (FIG. 15D). This strain constitutes a complete peptide-inducible lycopene biosynthetic pathway and the final component needed in order to achieve peptide biosensing is desired receptor and peptide secretion for biosensing communities.

The lycopene response was first validated in simple single-member A. fumigatus biosensor (yTC527, 2×CrtI). Using the lycopene readout, the limit of detection (LoD) of AfuPep in liquid culture was determined to be ˜30 nM.

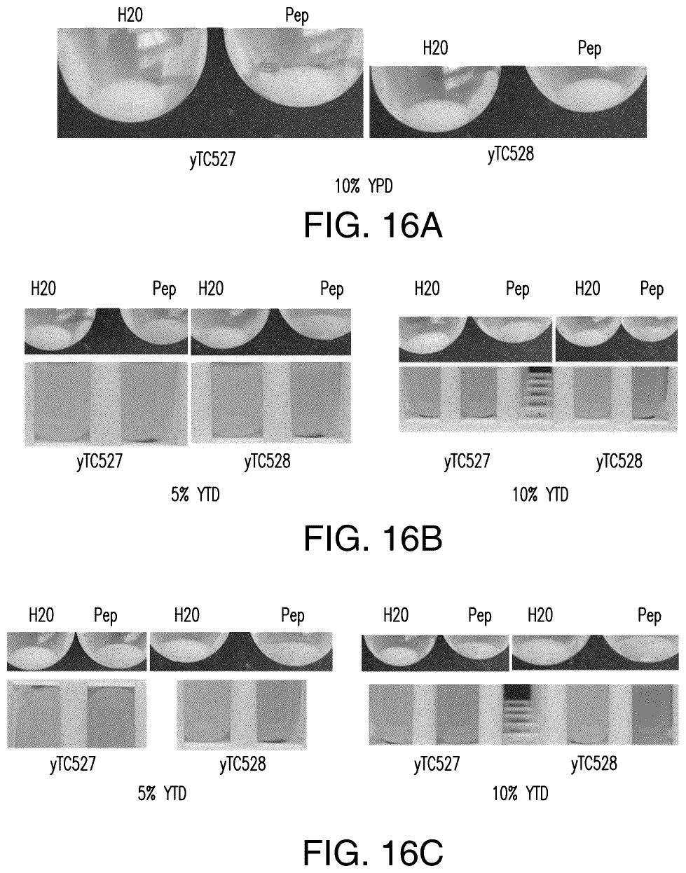

In order to achieve fast and robust lycopene color at very short timescales (within an hour), the culturing conditions of the improved lycopene strain (yTC590) were altered. For the previous lycopene biosensors, yeast complete synthetic medium was supplemented with 5% YPD in order to drive faster lycopene onset through rich supplementation. However, pushing higher YPD percentage could have led to lower lycopene color resolution due to similar orange hue of YPD and moderate basal lycopene production of the biosensing strain (Ostrov et al.). Interestingly, the lycopene strain activated through the disclosed optimized pheromone signaling pathway does not show any visible basal lycopene, so higher YPD supplementation was tested (FIGS. 16A-16C). By increasing YPD to 10% and by introducing a third copy of peptide-inducible CrtI (yTC528), the currently disclosed system showed an obvious color change in liquid culture after only two hours and in culture pellets a faint color was visible even after one hour incubation with 10 μM AfuPep (FIGS. 16A-16C).

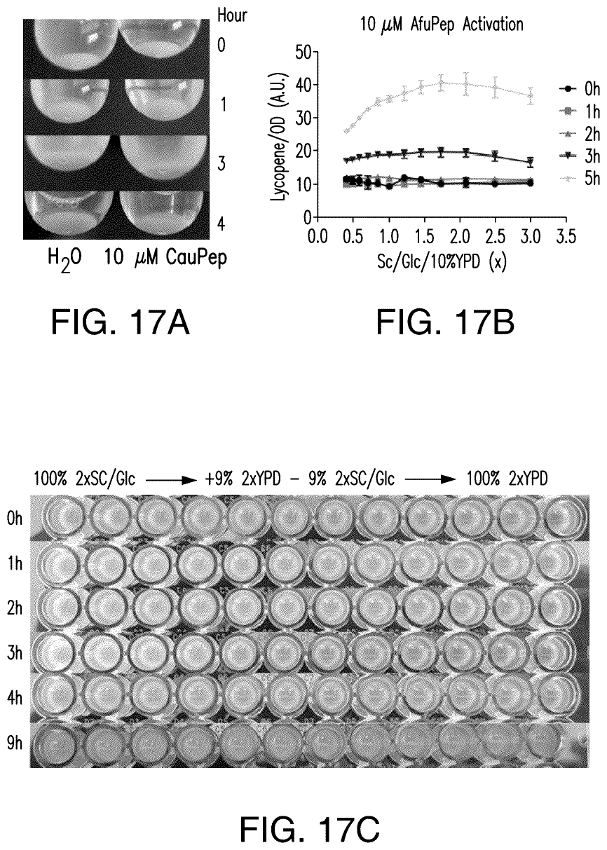

Three times concentrated standard complete media supplemented with 10% YPD (3×) as the highest concentration and did incremental dilutions with water thereof was validated (FIG. 17B). It was determined that the optimal media concentration is ˜2× with 15% increase in maximal lycopene intensity compared to 1×. Media concentration above 2× and below 1× gradually decreases maximal lycopene content. Next, YPD percentage in the total media used was improved. In the previous lycopene biosensors, yeast complete synthetic medium was supplemented with 5% YPD in order to drive faster lycopene onset through rich supplementation. However, pushing higher YPD percentage led to lower lycopene color resolution due to similar orange hue of YPD and moderate basal lycopene production of the biosensing strain (Ostrov et al.).

However, the lycopene strain activated through optimized pheromone signaling pathway does not show any visible basal lycopene (FIG. 14D), so higher YPD percentages were tested. By increasing YPD up to 30%, the YPD color did not impede with color contrast in liquid medium compared to pure SC/Glc media (FIG. 17C). With these results it was determined that optimal culturing conditions for robust lycopene color onset are 2×SC/Glc media supplemented with 30% 2×YPD.

Further experiments to improve the lycopene biosynthetic flux were performed to bring visible lycopene accumulation at shorter time. Initially, CrtI was replaced with CrtB (yTC472) under the control of pheromone-inducible promoter, since this enzyme is more efficient in yeast (R. Verwaal et al., “High-level production of beta-carotene in Saccharomyces cerevisiae by successive transformation with carotenogenic genes from Xanthophyllomyces dendrorhous,” Applied and Environmental Microbiology, vol. 73, no. 13, pp. 4342-4350, 2007). This should lead to faster response time after pheromone pathway induction and the visible accumulation of lycopene at lower CrtB concentrations. However, this setup led to constitutively orange strain non-responsive to peptide activation (FIG. 17A), so the optimization was continued with inducible CrtI only. Next, a third copy of the inducible CrtI (yTC528, 3×CrtI) was integrated. This led to one-hour improvement in visible color onset in liquid culture from three hours down to two hours (FIGS. 14B-C). It was then reasoned that the lycopene accumulation bottleneck lied further upstream in the biosynthetic pathway. First, the integration of a copy of strong constitutive HMG1 (HMG-CoA reductase) catalytic domain (yTC588, 3×CrtI+tHMG1) in a combination with downregulated ERG9 (squalene synthase) (yTC589, 3×CrtI+tHMG1−ERG9) was tested. ERG9 was downregulated by integrating a low expression level promoter pTEV1 upstream ERG9 ORF in its native locus. HMG1 converts HMG-CoA into mevalonic acid, which is a precursor in both the lycopene and squalene biosynthetic pathway, while ERG9 converts farnesyl pyrophosphate (FPP), yet another lycopene precursor, into squalene. By downregulating ERG9, an increase in the flux of FPP towards lycopene production rather than squalene was expected. Expression of a catalytic domain of HMG1 and disruption of ERG9 on top of usual lycopene biosynthetic pathway improved lycopene content seven-fold in previous study in Candida utilis (Shimada et al. Increased carotenoid production by the food yeast Candida utilis through metabolic engineering of the isoprenoid pathway. Appl. Environ. Microbiol. 64, 2676-2680 (1998)). The addition of tHMG1 did not lead to significant improvement in visible readout onset, but it did increase overall lycopene intensity by 15% and the ERG9 downregulation led to non-responsive strain.

Finally, to further increase visible lycopene accumulation onset, an additional copy of ERG20 (a FPP synthase) (yTC590, 3×CrtI+tHMG1+ERG20) was integrated into the strain, which slightly improved overall lycopene intensity (FIG. 14B). With the strain yTC590, improvement of the lycopene biosynthetic pathway was concluded. This strain shows AfuPep LoD at 50 nM in liquid culture (FIG. 14D). Finally, this strain shows 25% reduction in basal lycopene expression which is far below the visible threshold and 50% increase in the maximal lycopene content compared to previous biosensor strain with lycopene readout (yMJ260) reported by Ostrov et al. (FIG. 14E).

### Example 7: Detection of A. fumigatus in Patient Samples

The biosensor developed in Example 1 was next challenged with A. fumigatus patient samples received from the NIH. Clinical bronchoalveolar lavages of confirmed aspergillosis samples and negative samples will be sterile filtered and the eluent will be used directly for the biosensor activation.

The Aspergillus biosensor community described in Example 5 will be activated as a liquid culture with the patient eluent, or as a spotted dot on a paper which is then dipped into the patient eluent.

### Example 8: Development of a Live Yeast Biosensor for SARS-CoV-2

This example discloses the development of a live-yeast-based assay for detection of SARS-CoV-2. This assay is based on the detection of the spike (S) protein of SARS-CoV-2 through a heterologously expressed GPCR in Saccharomyces cerevisiae, which then transcriptionally activates the biosynthesis of a red pigment (lycopene) visible to the naked eye.

The desired protein biomarker for SARS-CoV-2 detection should be present at high concentrations in nasal swab samples obtained by non-medical users, be validated as a biomarker for SARS-CoV-2 diagnostics, and be well-characterized. The SARS-CoV-2 nucleocapsid protein was selected because it is the biomarker of choice for FDA authorized at-home lateral flow assays, and there are ˜700-2,200 copies of the nucleocapsid protein per virion particle. The SARS-CoV-2 Spike protein (S-protein) was also selected because it is a protruding protein on the virion capsid that mediates the coronavirus interaction with the human ACE2 receptor and infectivity it is highly exposed to the solvent and is the protein that includes clusters of variant mutations.

For the nucleocapsid protein, based on its 3D structural model, a linear epitope (residues 299-310) reported as a conserved T-cell and B-cell epitope was identified. This epitope lies in an exposed region of the C-terminal RNA binding domain, near the dimerization surface of the domain. An identical sequence can be found in the nucleocapsid protein of the SARS coronavirus, and it is remarkably similar to the homologous site in the MERS N-protein. A second epitope candidate was identified in a coiled region of the nucleocapsid protein near the C-terminal end of the domain (residues 335-346). This region showed a striking 42% identity with the Zygosaccharomyces rouxii mating peptide.

Because of the high similarities between the starting peptide and the target peptide, directed evolution of the GPCR towards recognition of a new ligand target can be achieved. A stepwise selection framework was used to progressively change the substrate specificity of proteins through directed evolution. This framework first involves selecting GPCRs from a sequence database with known peptide specificities and then looking for an intermediate ligand hit. These hits are characterized and used as parent receptors in new directed evolution cycles to generate hits against a more mature target ligand. This sequential peptide-based system is highly advantageous for directed evolution. A stable reporter strain was generated to perform directed evolution on plasmid-borne receptor variants. This strain is analogous to the lycopene reporter, where the lycopene biosynthetic module is replaced by the fluorescent reporter, mCherry. Variants of the whole receptor gene were generated by error-prone PCR (epPCR), transformed the plasmid library in yeast and grow the transformants in large flat metallic trays in semi-solid media. The mCherry reporter was then used to perform selections by fluorescence-activated cell sorting (FACS) and fluorescence-based microtiter plate screens. This screening platform decreases the directed evolution clonal selection time to less than three days and preserves both library complexity and clonal distribution. By repeating the procedure and selecting the nonfluorescent cells in uninduced conditions, constitutively active variants were counter selected. Multiple cycles of positive/negative selection ensure the removal of self-activating clones and promote a further enrichment of true positive. Next, single colonies were picked and challenged with the target ligand. The hits were sequenced, validated and used as parent receptors in new directed evolution cycles to generate hits against a more mature target ligand.

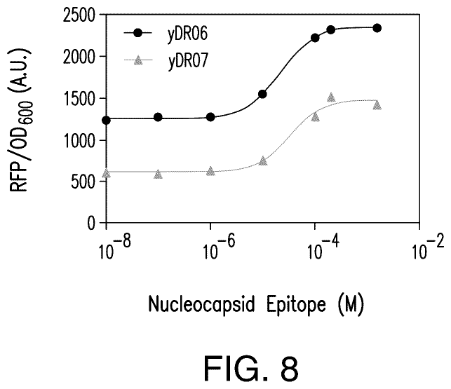

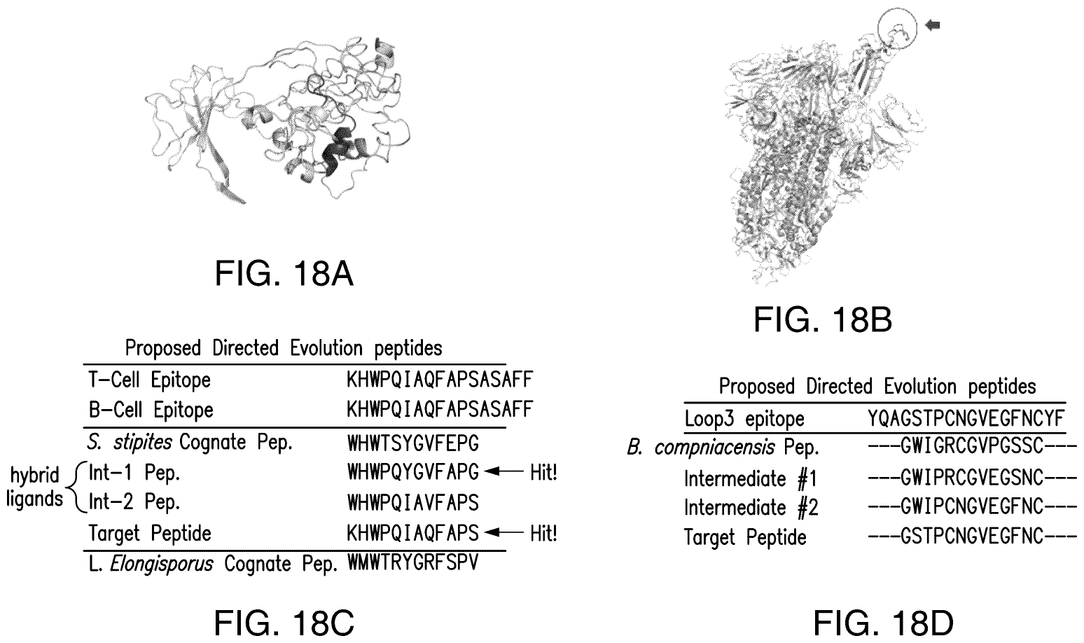

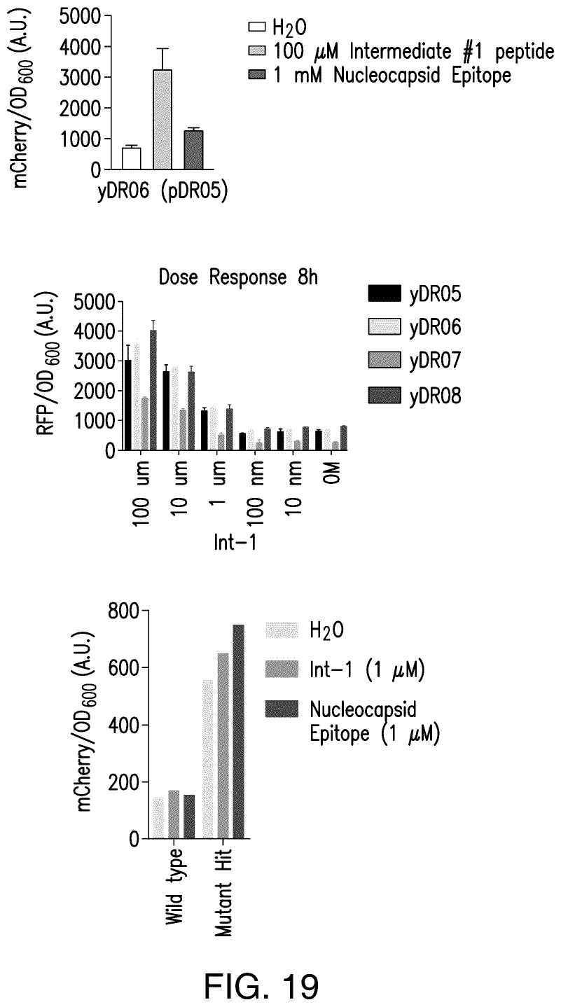

For the nucleocapsid biosensor, the sequence, KHWPQIAQFAPS (residues 299-310), was targeted as it shares four residues with the native peptide for the Scheffersomyces stipites GPCR receptor (SsSte2) (FIG. 18). With cryo-EM reconstruction of homologous receptors, the receptor activation critical contacts are mediated by the conserved His2-Trp3 pair, while the aromaticity of Phe9 mediates most of the interaction within the receptor binding pocket (FIG. 18A). The first intermediate connects the identical residues and modify the most divergent residue site, Thr4Pro. The second intermediate follows the same logic. To reach the final peptide, Trp1Lys, a common polymorphism at that position, and Val8Gln will be modified. Following the directed evolution strategy, libraries of SsSte2 mutants were mutated with an average error rate of 2.3±2.7 bases/kb. The SsSte2 receptor underwent mutagenesis, selection and screening twice. After the second round of directed evolution, the best hit shows a 3.4±0.3 fold increase in signal intensity in the presence of Intermediate-1 (FIG. 19). The EC₅₀ is ˜3.0 μM. Nucleotide sequences of example mutant SsSte2 are provided in Table 10. These hits will lead the iterative directed evolution process to further tailor the specificity and sensitivity to the target epitope. In addition, the Ste2 receptor underwent an additional round of mutagenesis, selection and screening, i.e., underwent mutagenesis, selection and screening three times (FIG. 8). The resulting evolved GPCR/biosensor strain has an EC₅₀ of ˜10.0 μM for the nucleocapsid epitope (FIG. 8). This result shows the validity of the directed evolution strategy and sets a path for engineering GPCRs/biosensors with low LoDs for the nucleocapsid protein/novel coronaviruses.

Alternatively, the L. Elongisporus receptor can be used for directed evolution towards the same epitopes and intermediates.

TABLE 10

| Name | Nucleotide Sequence |
|---|---|
| pDR04_mSsSte2 | TATACCGAAGGTCACGAAATTACTTTTTCAAAGCCGTCTCGAGCTAGCTTT |
| (yDR05) | CAGAAAAGTGAGAGGTCGTTTTGTTCATGAATGAAAGAGTTGCGGAAGATG |
| | GAAGTTGAGGAGAATTGTTGGCAGTTGCTGCCCAGATAGAGGATACTGGTA |
| | AAGACAAAACAACAAGCAAGTAACTTATAAGAATGAGCTGGCTATTCAGAG |
| | ATGTGCCATATCCGTAATGTACAAGAATGAGAATAGATGGGGCCAATAATG |
| | TTTGAGTAGCCATCATCAATAAGATGTGCAAACTGCCAAATTGCCGAAGGC |
| | CAAGGTATCTTCTTGTCTTTATGGCAAACCCTAACTTGAGAATCAAGATGA |
| | AAGACATTATGCTAACCGAAGTAGAAAAAAGTATCGTAGGCACATCTCTAA |
| | CCCAAGACCAGGTAGCGACCGTTGGCGTAGGAGTTCCATTGACAACTGAAT |
| | GATAAATTCTCGATTGTTGGATTGTACTATTGATAGTAAATCCTACAGCAG |
| | CAGCACCCGTGAATGCAGATATGGAGGTAAGAGCTATACCCAACTTCTTCA |
| | CTTCTGGTGATTGGAAGACGACGAACACCTGAAATGTCATGGACGCCTCGA |
| | TAAGAGCAACAACAATAACCTGGAAAGCATTGGTGGCTTCAGAGACAGAGA |
| | AATTGGTTTCTGGAGTTACCAAGCCGGAGAACGTAAAACTTAATCCAGCCA |
| | AAGGACCTAGCAAATATGCCAAATTAAGAGCAGACCGAATAATCATTGCAG |
| | TCAACGTAACCGAATTAAGATAGAAAGCTGGCGACTTCTTGCGTTTGACAT |
| | TGAAGATCAAAATAATTAAAAGCATTATGGTGCATGAACCAATGGAAACTC |
| | CATGGATAATGGCCTTTTGCATAGATTGGTTAACATATTCGTCAAAAGCTC |
| | CAAATGGGACACTAATTACTCTAGGATCATTTGGCAAGGTGTAGTTGACAA |
| | TGATATTCGCAGGGTTGAGAGTATTGATACTAGTATCCATTTTACTAGTAT |
| | CCGTCGAAACTAAGTTCTTGGTGTTTTAAAACTAAAAAAAAGACTAACTAT |
| | AAAAGTAGAATTTAAGAAGTTTAAGAAATAGATTTACAGAATTACAATCAA |
| | TACCTACCGTCTTTATATA |
| pDR05_mSsSte2 | GGTCACGAAATTACTTTTTCAAAGCCGTCTCGAGCTAGCTTTCAGAAAAGT |
| (yDR06) | GAGAGGTCGTTTTGTTCATGAATGAAAGAGTTGCGGAAGATGGAAGTTGAG |
| | GAGAATTGTTGGCAGTTGCTGCCCAGATAGAGGATACTGGTAAAGACAAAA |
| | CAACAAGCAAGTAACTTATAAGAATGAGCTGGCTATTCAGAGATGTGCCAT |
| | ATCCGTAATGTACAAGAATGAGAATAGATGGGGCCAATAATGTTTGAGTAG |
| | CCATCATCAATAAGATGTGCAAACTGCCAAATTGCCGAAGGCCAAGGTATC |
| | TTCTTGTCTTTATGGCAAACCCTAACTTGAGAATCAAGATGAAAGACATTA |
| | TGCTAACCGAAGTAGAAAAAAGTATCGTAGGCACATCTCTAACCCAAGACC |
| | AGGTAGCGACCGTTGGCGTAGGAGTTCCATTGACAACTGAATGATAAATTC |
| | TCGATTGTTGGATTGTACTATTGATAGTAAATCCTACAGCAGCAGCACCCG |
| | TGAATGCAGATATGGAGGTAAGAGCTATACCCAACTTCTTCACTTCTGGTG |
| | ATTGGAAGACGACGAACACCTGAAATGTCATGGACGCCTCGATAAGAGCAA |
| | CAACAATAACCTGGAAAGCATTGGTGGCTTCAGAGACAGAGAAATTGGTTT |
| | CTGGAGTTACCAAGCCGGAGAACGTAAAACTTAATCCAGCCAAAGGACCTA |
| | GCAAATATGCCAAATTAAGAGCAGACCGAATAATCATTGCAGTCAACGTAA |
| | CCGAATTAAGATAGAAAGCTGGCGACTTCTTGCGTTTGACATTGAAGATCA |
| | AAATAATTAAAAGCATTATGGTGCATGAACCAATGGAAACTCCATGGATAA |
| | TGGCCTTTTGCATAGATTGGTTAACATATTCGTCAAAAGCTCCAAATGGGA |
| | CACTAATTACTCTAGGATCATTTGGCAAGGTGTAGTTGACAATGATATTCG |
| | CAGGGTTGAGAGTATTGATACTAGTATCCATTTTACTAGTATCCGTCGAAA |
| | CTAAGTTCTTGGTGTTTTAAAACTAAAAAAAAGACTAACTATAAAAGTAGA |
| | ATTTAAGAAGTTTAAGAAATAGATTTACAGAATTACAATCAATACCTACCG |
| | TCTTTATA |
| pDR06_mSsSte2 | TCACGAAATTACTTTTTCAAAGCCGTCTCGAGCTAGCTTTCAGAAAAGTGA |
| (yDR07) | GAGGTCGTTTTGTTCATGAATGAAAGAGTTGCGGAAGATGGAAGTTGAGGA |
| | GAATTGTTGGCTGTTGCTGCCCAGATAGAGGATACTGGTAAAGACAAAACA |
| | ACAAGCAAGTAACTTATAAGAATGAGCTGGTTATTCAGAGATGTGCCATAT |
| | CCGTAATGTACAAGAATGAGAATAGATGGGGCCAATAATGTTTGAGTAGCC |
| | ATCATCAATAAGATGTGCAAACTGCCAAATTGCCGAAGGCCAAGGTATCTT |
| | CTTGTCTTTATGGCAAACCCTAACTTGAGAATCAAGATGAGAGACATTATG |
| | ATAACCGAAGTAGAAAAAAGTATCGTAGGCACATCTCTAACCCAAGACCAG |
| | GTAGCGACCGTTGGCGTAGGAGTTCCATTGACAACTGAATGATAAATTCTC |
| | GATTGATGGATTGTACTATTGATAGTAAATCCTACAGCAGCAGCACCCGTG |
| | AATGCAGATATGGAGGTAAGAGCTATACCCAACTTCTCCACTTCTGGTGAT |
| | TGGAAGACGACGAACACCTGAAATGTCATGGACGCCTCGATAAGAGCAACA |
| | ACAATAACCTGGAAAGCATTGGTGGCTTCAGAGACAGAGAAATTGGTTTCT |
| | GGAGTTACCAAGCCGGAGAACGTAAAACTTAATCCAGCCAACGGACCTAGC |
| | AAATATGCCAAATTAAGAGCAGACCGAATAATCATTGCAGTCAACGTAACC |
| | GAATTAAGATAGAAAGCTGGCGACTTCTTGCGTTTGACATTGAAGATCAAA |
| | ATAATTAAAAGCATTATGGTGCATGAACCAATGGAAACTCCATGGATAATG |
| | GCCTTTTGCATAGATTGGTTAACATATTCGTCAAAAGCTCCAAATGGGACA |
| | CTAATTACTCTAGGATCATTTGGCAAGGTGTAGTTGACAAGAAAATCCGCA |
| | GGGTTGAGAGTATTGATACTAGTATCCATTTTACTAGTATCCGTCGAAACT |
| | AAGTTCTTGGTGTTTTAAAACTAAAAAAAAGACTAACTATAAAAGTAGAAT |
| | TTAAGAAGTTTAAGAAATAGATTTACAGAATTACAATCAATACCTACCGTC |
| | TTTATATACT |
| pDR07_mSsSte2 | CGTCTCGAGCTAGCTTTCAGAAAAGTGAGAGGTCGTTTTGTTCATGAATGA |
| (yDR08) | AAGAGTTGCGGAAGATGGAAGTTGAGGAGAATTGTTGGCAGTTGCTGCCCA |
| | GATAGAGGATACTGGTAAAGACAAAACAACAAGCAAGTAACTTATAAGAAT |
| | GAGCTGGCTATTCAGAGATGTGCCATATCCGTAATGTACAAGAATGAGAAT |
| | AGATGGGGCCAATAATGTTTGAGTAGCCATCATCAATAAGATGTGCAAACT |
| | GCCAAATTGCCGAAGGCCAAGGTATCTTCTTGTCTTTATGGCAAACCCTAA |
| | CTTGAGAATCAAGATGAAAGACATTATGCTAACCGAAGTAGAAAAAAGTAT |
| | CGTAGGCACATCTCTAACCCAAGACCAGGTAGCGACCGTTGGCGTAGGAGT |
| | TCCATTGACAACTGAATGATAAATTCTCGATTGTTGGATTGTACTATTGAT |
| | AGTAAATCCTACAGCAGCAGCACCCGTGAATGCAGATATGGAGGTAAGAGC |
| | TATACCCAACTTCTTCACTTCTGGTGATTGGAAGACGACGAACACCTGAAA |
| | TGTCATGGACGCCTCGATAAGAGCAACAACAATAACCTGGAAAGCATTGGT |
| | GGCTTCAGAGACAGAGAAATTGGTTTCTGGAGTTACCAAGCCGGAGAACGT |
| | AAAACTTAATCCAGCCAAAGGACCTAGCAAATATGCCAAATTAAGAGCAGA |
| | CCGAATAATCATTGCAGTCAACGTAACCGAATTAAGATAGAAAGCTGGCGA |
| | CTTCTTGCGTTTGACATTGAAGATCAAAATAATTAAAAGCATTATGGTGCA |
| | TGAACCAATGGAAACTCCATGGATAATGGCCTTTTGCATAGATTGGTTAAC |
| | ATATTCGTCAAAAGCTCCAAATGGGACACTAATTACTCTAGGATCATTTGG |
| | CAAGGTGTAGTTGACAATGATATTCGCAGGGTTGAGAGTATTGATACTAGT |
| | ATCCATTTTACTAGTATCCGTCGAAACTAAGTTCTTGGTGTTTTAAAACTA |
| | AAAAAAAGACTAACTATAAAAGTAGAATTTAAGAAGTTTAAGAAATAGATT |
| | TACAGAATTACAATCAATACCTACCGTCTTTATATA |

TABLE 11

| | | | Cognate Mating |
|---|---|---|---|
| Species | | | Peptide |
| Sc | Saccharomyces | cerevisiae | 1-WHWLQLKPGQPMY |
| Sca | Saccharomyces | castellii | 1-NWHWLRLDPGQPLY |
| Vp2 | Vanderwaltozyma | polyspora2 | 1-WHWLRLRYGEPIY |
| Vp2 | Vanderwaltozyma | polyspora2 | 2-PWHWLRLRYGEPIY |
| Vp1 | Vanderwaltozyma | polysporal | 1-WHWLELDNGQPIY |
| Td | Torulaspora | delbrueckii | 1-GWMRLRLGQPL |
| Td | Torulaspora | delbrueckii | 2-GWMRLRLGQPM |
| Td | Torulaspora | delbrueckii | 3-GWMRLRIGQPL |
| Sk | Saccharomyces | kluyveri | 1-WHWLSFSKGEPMY |
| Sk | Saccharomyces | kluyveri | 2-PWHWLSFSKGEPMY |
| Kl | Kluyveromyces | lactis | 1---WSWITLRPGQPIF |
| Kl | Kluyveromyces | lactis | 2-SPWSWITLRPGQPIF |
| Zr | Zygosaccharomyces | rouxii | 1-HFIELDPGQPMF |
| Zr | Zygosaccharomyces | rouxii | 2-AHFIELDPGQPMF |
| Zb | Zygosaccharomyces | bailii | 1-HLVRLSPGAAME |
| Zb | Zygosaccharomyces | bailii | 2-PLVRLSPGAAMF |
| Zb | Zygosaccharomyces | bailii | 3-APLVRLSPGAAMF |
| Zb | Zygosaccharomyces | bailii | 4-AHLVRLSPGAAMF |
| Cg | Candida | glabrata | 1-WHWVRLRKGQGLF |
| Cg | Candida | glabrata | 2-WHWVKIRKGQGLF |
| Ag | Ashbya | gossypii | 1-WFRLSLHHGQSM |
| Ss | Scheffersomyces | stipitis | 1-WHWTSYGVFEPG |
| Ss | Scheffersomyces | stipitis | 2-PWHWTSYGVFEPG |
| Kp | Komagataella | pastoris | 1-FRWRNNEKNQPFG |
| | (Pichia) | | |
| Cgu | Candida (Pichia) | guilliermondii | 1-KKNSRFLTYWFFQPIM |
| Cp | Candida | parapsilosis | 1-KPHWTTYGYYEPQ |
| Cau | Candida | auris | 1-KWGWLRFFPGEPFV |
| Yl | Yarrowia | lipolytica | 1-WRWFWLPGYGEPNW |
| Cl | Candida (Clavispora) | lusitaniae | 1-KWKWIKFRNTDVIG |
| Cl | Candida (Clavispora) | lusitaniae | 2---WGWIHFLNTDVIG |
| Cl | Candida (Clavispora) | lusitaniae | 3-PKWKWIKFRNTDVIG |
| Ca | Candida | albicans | 1-GFRLTNFGYFEPG |
| Ct | Candida | tropicalis | 1-KFKFRLTRYGWFSPN |
| Cn | Candida | tenuis | 1-FSWNYRLKWQPIS |
| Le | Lodderomyces | elongisporous | 1----WMWTRYGRFSPV |
| Le | Lodderomyces | elongisporous | 2-DPGWMWTRYGRFSPV |
| Gc | Geotrichum | candidum | 1-GDWGWFWYVPRPGDPAM |
| Gc | Geotrichum | candidum | 2-PGDWGWFWYVPRPGDPAM |
| Bm | Baudoinia | compniacensis | 1-GWIGRCGVPGSSC |
| So | Schizosaccharomyces | octosporus | 1-----TYEDFLRVYKNWWS |
| | | | FQNPDRPDL |
| So | Schizosaccharomyces | octosporus | 2-PACTTYEDFLRVYKNWWS |
| | | | FQNPDRPDL |
| Tm | Tuber | melanosporum | 1-WTPRPGRGAY |
| Ao | Aspergillus | oryzae | 1-WCALPGQGC |
| Sp | Schizosaccharomyces | pombe | 1-TYADELRAYQSWNTFVNP |
| | | | DRPNL |
| Sp | Schizosaccharomyces | pombe | 2-KTYADFLRAYQSWNTFVN |
| | | | PDRPNL |
| Af | Aspergillus | fischeri | 1-WCHLPGQGC |
| | (Neosartorya) | | |
| Pd | Pseudogymnoascus | destructans | 1---FCWRPGQPCG |
| Pd | Pseudogymnoascus | destructans | 2---FCQRPGQLCG |
| Pd | Pseudogymnoascus | destructans | 3-LEFGGLEKEQNS |
| Sj | Schizosaccharomyces | japonicus | 1----VSDRVKQMLSHWWNF |
| | | | RNPDTANL |
| Sj | Schizosaccharomyces | japonicus | 2-PERRVSDRVKQMLSHWWN |
| | | | ERNPDTANL |
| Pb | Paracoccidioides | brasiliensis | 1-WCTRPGQGC |
| Mg | Mycosphaerella | graminicola | 1-GNSFVGWCGAIGAPCA |
| Mg | Mycosphaerella | graminicola | 2-------WCGAIGAPCA |
| Pr | Penicillium | chrysogenum | 1-WCGHIGQGC |
| Pr | Penicillium | chrysogenum | 2-KWCGHIGQGC |
| An | Aspergillus | nidulans | 1-WCRFRGQVCG |
| Sn | Phaeosphaeria | nodorum | 1-KYNGWRYRPYGLPVG |
| Hj | Hypocrea | jecorina | 1-WCYRIGEPCW |
| Hj | Hypocrea | jecorina | 2-WCWILGGKCW |
| Bc | Botrytis | cinerea | 1-WCGRPGQPC |
| Bb | Beauvaria | bassiana | 1-WCMRPGQPCW |
| Bb | Beauvaria | bassiana | 2-WCMQTPKCW |
| Nc | Neurospora | crassa | 1-QWCR---IHGQSCW |
| Nc | Neurospora | crassa | 2-QVCNMRLHPKKVCW |
| She | Sporothrix | scheckii | 1---YCPLKGQSCW |
| She | Sporothrix | scheckii | 2-QRYCPLKGQSCW |
| Mo | Magnaporthe | oryzea | 1-QWCPRRGQPCW |
| Dh | Dactylellina | haptotyla | 1-WCVYNSCP |
| Fg | Fusarium | graminearum | 1-WCWWKGQPCW |
| Fg | Fusarium | graminearum | 2-WCTWKGQPCW |
| Cc | Capronia | coronata | 1-GLSYWKGVNDGGSS |

For the S protein, loop3 (residues 472-490; GSTPCNGVEGFNC) in the receptor binding domain (RBD) was identified as the epitope of interest. The S-protein is naturally protruding out of the virion capsid, and loop3 is the most flexible part of the protein, mediating the contact with the ACE2 receptor. Several human neutralizing antibodies are shown to prevent infection by binding to loop3 and coating the RBD, thus disrupting binding to the ACE2 receptors, proving that the loop can be detected and bound when the virion is still intact.

For the S protein biosensor, the Baudoinia compniacensis mating peptide (GWIGRCGVPGSSC) has five residues identical to the loop3 (residues 476-488) in the RBD of the S-protein (FIG. 18B). The first intermediate (GWIPRCGVEGSNC) was designed to address the substitutions involving prolines (Gly4Pro, Pro9Glu). The second intermediate (GWIPCNGVEGFNC) shifts the Cys6 to Cys5 and connects everything apart from the N-terminal residues. Cys6 is part of a conserved pair of cysteines featured in B. compniacensis mating pheromone which forms a disulfide bridge, forcing a loop in the peptide. An identical pair of cysteines lock the structure of the loop3 in the S-protein in a similar conformation. To achieve the final peptide, Trp2-Ile3 are substituted with Ser2-Thr3 (FIG. 18C). Alternatively, C. albicans Ste2, whose mating peptide GFRLTNFGYFEPG, can be evolved to bind loop4 (GFQPTNGVGYQPYR) in the spike RBD.

In addition, proteolytic enzymes can be used to separate the peptide epitope from the protein of interest and the resulting fragments can be detected using the yeast sensor. Arg-C proteinase is able to detach the target linear epitope from the N-protein while trypsin can be used to generate 6 high-profile peptide targets from the S-protein (fragment 409-417 (QIAPGQTGK): 44% identical to S. cerevisiae mating peptide, fragment 103-113 (GWIFGTTLDSK): 54% identical to C. lusitaniae mating peptide, fragments 79-97 (FDNPVLPFNDGVYFASTEK), 159-182 (VYSSANNCTFEYVSQPFLMDLEGK), 215-237 (DLPQGFSALEPLVDLPIGINITR) and 635-646 (VYSTGSNVFQTR)). For these scenarios, the cleaving protease can be either secreted by the yeast or included as a lyophilized enzyme in the biosensor kit.

The SARS-CoV-2 biosensors will be validated using dose response assays performed with serial 10-fold dilutions of the target peptide epitope (100 μM-10 pM), quantifying EC₅₀ and S/N. Samples with no added peptide, intermediate peptides or the original yeast pheromone will be used as control. For analytical specificity, homologous peptides from other known human coronaviruses (OC43, 229E, NL63, HKU, SARS, and MERS) and SARS-CoV-2 variants of concern (B.1.1.7 (Alpha), B.1.351 (Beta), P1 (Gamma), and B.1.617.2 (Delta)) will be tested.

Sequencing several positive GPCR plasmid vectors will identify the mutations relevant for the increasing substrate specificity. Beneficial mutations from different hits will be combined aiming for a better response. The GPCR of the best responding hits will be integrated in the biosensor strain with the lycopene readout and the peptide-based characterization will be repeated in settings that mirror the product prototype intended use.

### Example 9: Development of a Live Yeast Biosensor for Ebola

This example discloses the development of a live yeast-based assay for detection of an ebolavirus. Ibis assay is based on the detection of the small secreted glycoprotein or the VP40 matrix protein of the ebolavirus through a heterologously expressed GPCR in Saccharomyces cerevisiae, which then transcriptionally activates the biosynthesis of a red pigment (lycopene) visible to the naked eye. This biosensor is low-cost because once the yeasts have been engineered, the fermentation and drying can be scaled locally and the distribution and storage is at room temperature. The biosensor is low-tech because no equipment, reagents other than water and sugar, or technical know-how are required to run the test.

The small secreted glycoprotein (sGP) was identified as the primary biomarker for the Ebola biosensor because it is the most highly conserved protein for Ebola and can be detected in infected patients' bodily fluids such as sera, saliva, tears and urine. In addition, there are antibody-based diagnostics for sGP, validating its utility. ELISA—with the limit of detection in the μM-pM range—has proved successful for detection of Ebola antigens in serum with 10²-10³ PFU/ml, which correspond to the early stages of the ebolavirus infection. Previous reports suggest that the Ebola antigen ELISA can be used for detection using oral fluid samples and that the oral fluids are only 64 times less concentrated in Ebola antigens than the serum.

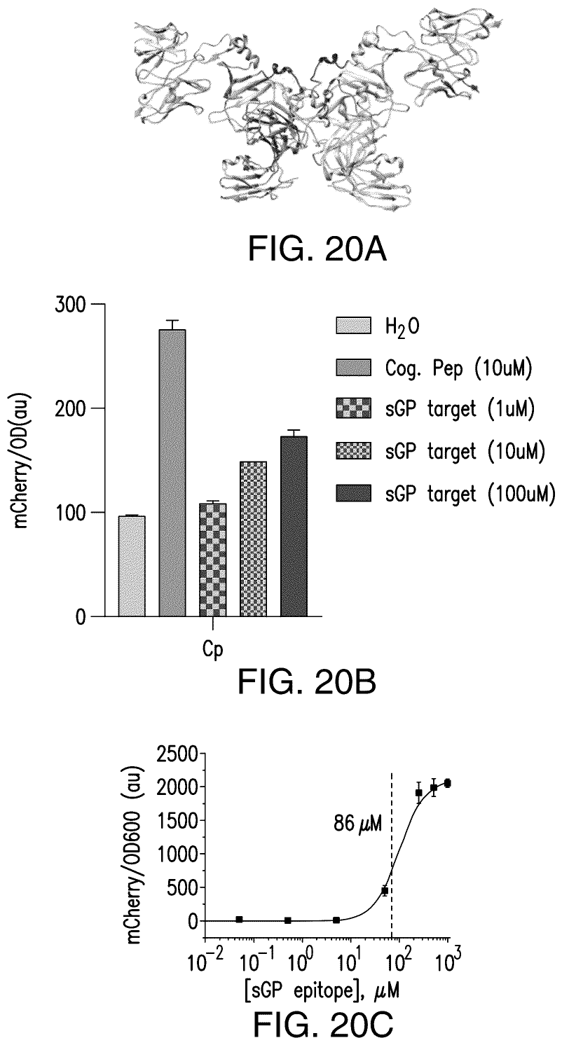

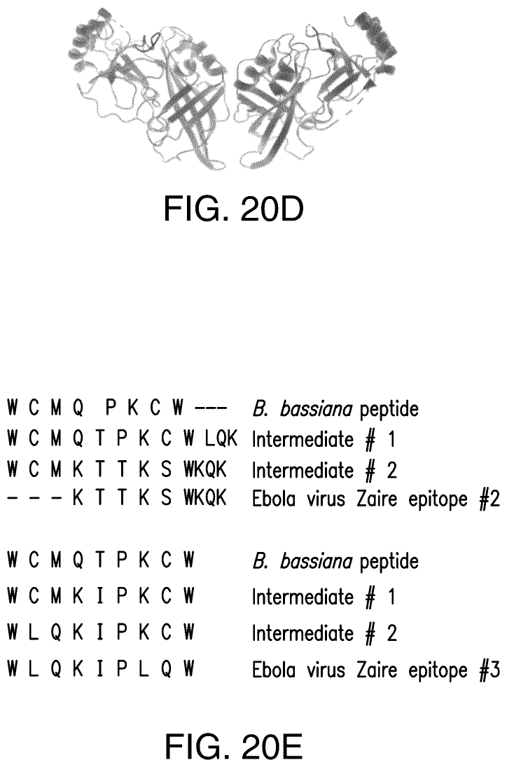

An exposed loop region containing a conserved linear epitope reported in the Immune Epitope Database after inspection of the high-resolution x-ray crystal structure of the sGP. Promisingly, the linear epitope VNATEDPSSGYY residues 203-214 from Ebola Zaire has three consecutive amino acids identical with the native mating peptide for Candida parapsilosis (CpSte2) receptor (FIG. 20A). It was found that native CpSte2 bound the target sGP peptide, although 10-fold higher concentrations were needed to induce expression levels equivalent to the native peptide (FIG. 20B). As shown in FIG. 20C, native CpSte2 bound to the target sGP epitope with an EC₅₀ of 86 μM, ˜1000-fold less compared to the cognate peptide. Using directed evolution strategy as described in Example 1, an error-prone PCR library of CpSte2 mutants with an average error rate of four mutations per gene were generated.

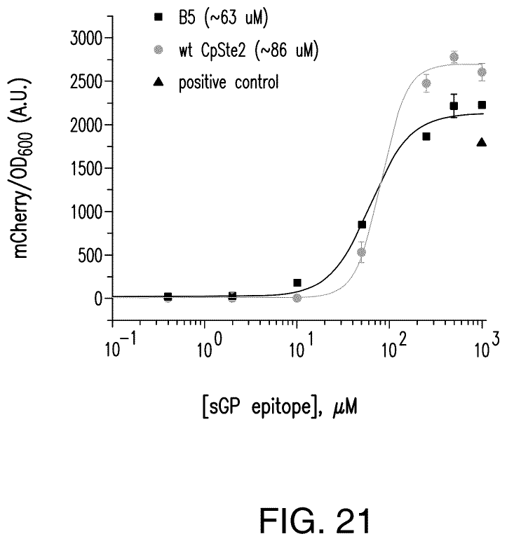

Following this directed evolution strategy, libraries of CpSte2 mutants with an error rate of 5±2 bases/kb were created. The CpSte2 receptor underwent mutagenesis, selection, and screening once. After the first round of directed evolution, the best hit shows a ˜1.4-fold increase in EC₅₀ for sGP target peptide compared to the wild-type receptor (FIG. 21). Rounds of mutagenesis, selection, and characterization with hits from this first round are being performed.

As illustrated in FIG. 20C, a direct hit for the sGP epitope VNATEDPSSGYY was identified and did not require the use of intermediate peptides. For the sGP epitopes 2 (KTTKSWLQK (residues 337-345)) and 3 (WLQKIPLQW (residues 342-350)), intermediated were designed based on the native mating peptide for B. bassiana (BbSte2), as these epitopes share the most similarities with BbSte2 (FIG. 20E). Analogous intermediates for all the alternative epitopes were designed. If needed, any of the peptide intermediates can be modified in the course of DE.

One of the big issues with the antigen detection systems currently available, is the occurrence of false positive results. A GPCR/biosensor will be engineered to recognize three different epitopes on sGP. These orthogonal biosensors in a test strip will minimize false positives and negatives. In addition to the VNATEDPSSGYY epitope, the linear epitopes KTTKSWLQK (residues 337-345 from the sGP of Zaire ebolavirus) and WLQKIPLQW (residues 342-350 from the sGP of Zaire ebolavirus), which epitopes are unique to the Zaire ebolavirus, are tested. A similar directed evolution strategy will be used as described herein to evolve GPCRs/biosensors for these two sGP Ebola epitopes.

Biosensors for the detecting the VP40 protein the ebolavirus are designed. VP40 is the most abundant protein in Ebola virus Zaire at 8391 copies per virion. WTDDTPTGSNGA (residues 191-202) was identified as a potential epitope for biosensor (FIG. 20D), as this region of VP40 reacted strongly with serum from asymptomatic EVD individuals. Moreover, this epitope is unique for Ebola virus Zaire after comparing the sequence of Ebola virus Zaire VP40 to the homologous proteins in Bundibugyo and Sudan species. VP40 has been previously expressed and purified in E. coli. For the ease of comparison of the commercial tests' performance, the approximated LoDs in nM are indicated in the parentheses next to the values reported by the manufacturers.

As an alternative, an Arg-C proteinase or trypsin will be employed to separate the target epitope, e.g., VNATEDPSSGYY, from the sGP. For these scenarios, the cleaving protease can be secreted by the yeast biosensor or included as an enzyme in the biosensor kit. Lyophilized Arg-C proteinase is stable at 2° C. to 8° C. until the expiration date which would allow for practical integration into the biosensor kit design. In addition, virion can be cleaved to release encapsulated VP40 by addition of SDS (up to 0.01%) and Tween20 (up to 0.05%) which did not affect the vitality of the yeast biosensor.

### Example 10: Development of a Live Yeast Biosensor for Multiple Ebolavirus Species

To develop a robust Ebola diagnostic, additional receptors will be developed to detect redundant epitopes and distinguish between the five different ebolavirus species, e.g., Zaire, Bundibugyo, Sudan, Reston and Tai Forest. Redundant receptors for the Zaire sGP will be developed by using multiple conserved B- and T-cell epitope predictions. These epitopes will be screened against the collection of fungal GPCRs to establish a parent receptor for the stepwise Directed evolution (DE) approach. In a similar approach, the linear epitope region in the sGP will be targeted to create redundant receptors for the predicted conserved B- and T-cell epitopes of each respective ebolavirus species. While the Zaire ebolavirus is the most dangerous species, responsible for the highest number of outbreaks and mortality rates in humans, biosensors for other ebolavirus species will be constructed as well.

Two main types of biosensors for Ebola detection are constructed. The first is a genus-specific biosensor that would generate a positive outcome in response to any species within the genus ebolavirus. The second type of biosensor is a species-specific biosensors that would generate positive outcomes only in response to the presence of specific species within the genus ebolavirus. The difference between these two types of biosensors is simply the choice of target epitopes. For the construction of genus-specific biosensors, target epitopes conserved across the genes and exist in all species will be chosen. These epitopes are GFRSGVPPKVVNYE (residues 86-100), FHKEGAFFLYDRL (residues 153-165), and KEGAFFLYDRLAST (residues 155-168). For the construction of species-specific biosensors, epitopes that are unique to a species of interest will be chosen. After performing multiple sequence alignment of sGP sequences of Zaire, Bundibugyo and Sudan viruses, two least conserved regions of sGP which are LPTQGPTQQLK (residues 325-337) and QTEPKTSVV (residues 311-320) were identified. These epitopes are screened against the collection of fungal GPCRs to establish a parent receptor for the stepwise DE approach.

### Example 11: Development of a Live Yeast Biosensor Product Prototype for Detecting Viruses

This example describes the prototype for the daily at-home testing for viruses, e.g., respiratory viruses such as SARS-CoV-2, by the consumer. This prototype reinforces the unique advantages of the living yeast biosensor in terms of cost, visible read-out, scalability and easy shipping and storage.

Six at-home SARS-CoV-2 diagnostic tests using self-collected nasal swab samples have presently received EUA for PoC home testing. Four of these tests are lateral flow assays detecting the viral nucleocapsid (Abbott, Ellume, Quidel, and OraSure), and the other two are based on the Loop-mediated isothermal amplification (LAMP) technology for amplification of DNA (Lucira and Cue). The Lucira, Abbott, and Ellume tests were examined. These tests were sophisticated, onerous and not straightforward for a layperson consumer. The nasal swab is introduced to a stand-alone, sleek vessel that communicates with one's phone (Ellume) or in a miniaturized DNA amplification device (Lucira), or it is threaded into a folded-up paper platform where water is dropped to initiate the lateral flow process (Abbott). With their relative complexity, and high cost ($20-$50/test), these tests seem unlikely to evolve into a daily, or even weekly, home test.

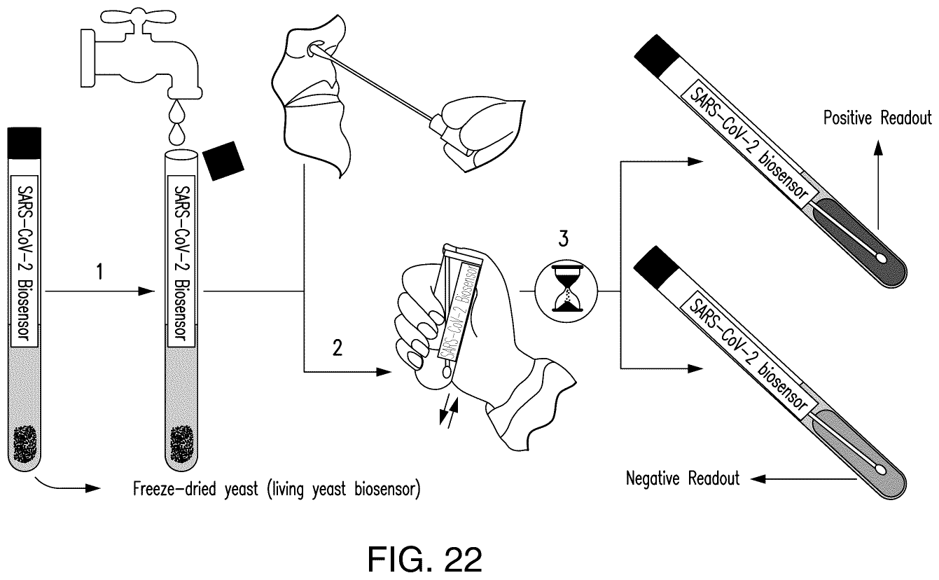

The currently disclosed biosensor provides a simpler alternative that is intuitive and requires minimal materials, thereby enabling large-scale deployment to the PoC or daily at-home testing by a non-expert at a low cost. The living yeast biosensor will be formulated as dried yeast packaged in a plastic test tube (FIG. 22). The sealed test tube will ensure the long-term integrity of the yeast while providing ease of use by simply opening the tube's cap by the end user. Significantly, this product design allows obtaining the nasal swab sample using the same protocol(s) already optimized for the EUA PoC SARS-CoV-2 diagnostic tests. The tube containing the yeast can be stored at home at room temperature. When ready to use, water is added to the test tube (e.g., about 500 μl of water), activating the yeast; the nasal swab sample is introduced into the test tube, the cap closed, the tube shaken, and finally, the user waits for a color change. If the SARS-CoV-2 biomarkers are present, they will activate the yeast GPCRs, turning on the lycopene read-out, and causing the solution to change color in case of a positive response (FIG. 22).

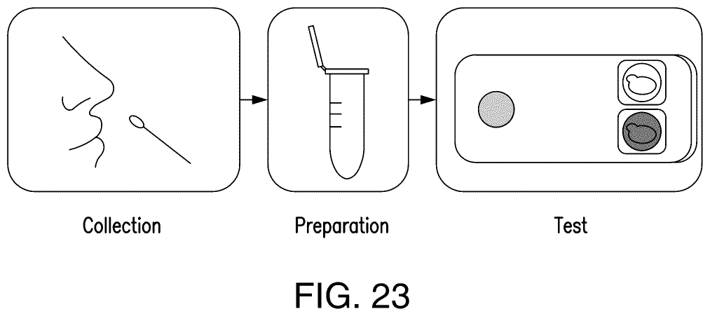

If the intact virion is proven unable to activate the biosensor, powdered viral lysis buffer to the dry yeast can be added at the bottom of the tube to solubilize the viral proteins or a processing step can be added where the lysis solution is added on the swab. It was confirmed that an SDS concentration up to 0.01% and Tween20 up to 0.05% does not impact the vitality and function of the presently disclosed live yeast biosensor. For example, the yeast biosensor can be stored at room temperature for at least 38 weeks. Moreover, the yeast sensor strain can be engineered to secrete a protease that cleaves the peptide epitope from the viral protein, increasing availability to interact with the GPCR receptor. As a backup strategy for the general format, the same product design of the at-home lateral flow assays already approved by the FDA to diagnose SARS-CoV-2 can be used. FIG. 23 provides an additional exemplary prototype that is a lateral flow assay.

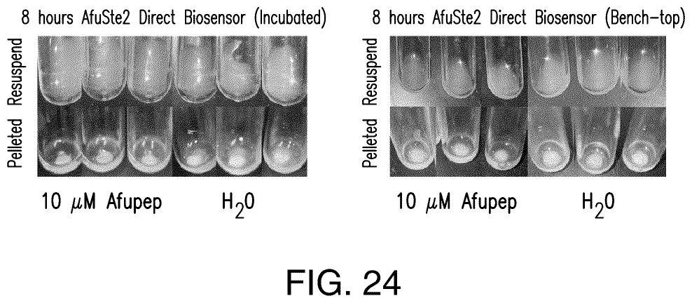

To determine whether the yeast biosensor can detect the targeted pathogen analyte in the presence of the nasal swab solution. The yeast biosensor's ability to detect its cognate fungal biomarker in the presence of the nasal swab solution was tested. Results proved that the yeast biosensor is active, robust, and does not interfere with the matrix components of the biospecimen. This experiment involved a biosensor containing an Ste2 homolog from Aspergillus fumigatus (AfuSte2) that produces lycopene in response to its cognate pheromone peptide (AfuPep). Indeed, this biosensor functions properly in the presence of human biospecimens and produces lycopene only when AfuPep is present (FIG. 24). Experiments were carried out at yeast standard growth conditions, which include at 30° C.; experiments also needed to be conducted at at-home-like temperatures to ensure that the biosensor is viable and active in these settings. When incubated at room temperature, the yeast still produced an observable difference in lycopene compared to the water negative control (FIG. 24).

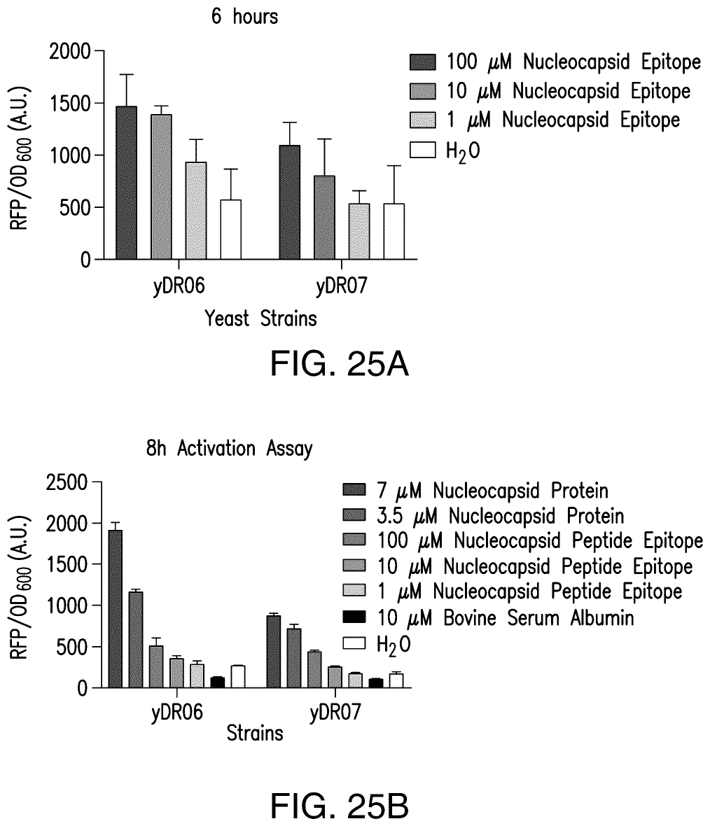

Confirming that the yeast biosensor is viable and active in the presence of nasal swab solutions, the coronavirus biosensor described in Example 8 was tested under the same conditions. When the nucleocapsid epitope was added to the nasal swab solution, an increased signal readout in a dose dependent manner was observed, confirming that the coronavirus yeast biosensor can recognize the nucleocapsid epitope in the presence of a human biospecimen. With a final nucleocapsid protein epitope concentration of 100 μM, the biosensor signal was over twice as strong compared to its signal in the presence of water (FIG. 25). All original nasal swab samples were diluted 5 times in these experiments to allow for the proper amount of yeast biosensor and yeast growth media to be added to the reaction mixture that keeps the yeast biosensor functional. These data show that the coronavirus yeast biosensor was able to detect coronavirus antigens from nasal swab samples.

The performance of the prototype biosensors will be assessed using a test regimen analogous to a formal application for FDA EUA clearance. Specifically, the tentative limit of detection (LoD) of the device will be measured with serial dilutions of purified target protein(s), inactivated virus, and contrived swab specimens using authentic live virus. LoD measurements will be performed with serial 10-fold dilution rows of the purified target protein(s) (100 nM-10 μM), inactivated virus (10⁵-10⁰ genome equivalents (GE)/ml) and live virus (10³-10⁰ TCID50/ml). The inactivated virus will be quantitated by Triplex CII-SARS-CoV-2 rRT-PCR. Dilutions are prepared in water. Each dilution will be run in triplicate, and the threshold for positivity is 3/3.

Thereafter, the reproducibility of detection with 95% confidence at 3×LoD will be demonstrated. Reproducibility of detection will be determined with 20 replicates at 3×LoD (>18 positives ≥95% confidence). Dilutions for reproducibility will use water and water+nasal swab/oral swab; 20 replicates in each diluent will be prepared.

Next, differential specificity of the assay will be shown by testing other human coronaviruses as well as common respiratory pathogens. To test the specificity of the SARS-CoV-2 biosensor, it will be tested on other known human coronaviruses (OC43, 229E, NL63, HKU, SARS, and MERS; all available at CII (SARS at the BSL-3 facility, which is registered for Possession, Use and Transfer of Select Biological Agents and Toxins)). Moreover, common respiratory pathogens will be tested for lack of cross-reactivity at 3× assay LoD, 10× assay LoD and 100× assay LoD (GE/ml) including, FLUAV, FLUBV, RSV, HPIV1-4, MPV, ADV, RV, EV-D68, S. pneumoniae, H. influenzae, M. catarrhalis. In an additional/extended assay distinguishing between SARS-CoV, SARS-CoV-2 and SARS-CoV-2 variants of concern will also be completed. Variants B.1.1.7 (Alpha), B.1.351 (Beta), P1 (Gamma), and B.1.617.2 (Delta) for these assays are available at CII. FDA-provided QC panels for SARS-CoV-2/COVID-19 assay validation will also be included once they become available.

Finally, 50 negative and 50 positive authentic clinical specimens, all confirmed by certified gold-standard qPCR, will be analyzed to assess the diagnostic sensitivity and specificity of the assay. A positive outcome is a biosensor with performance on par with the currently-approved lateral flow assays (>85% sensitivity, >98% specificity, and LoD˜20 pM (estimated from their EUA)). To assess diagnostic sensitivity and specificity of the assay, 50 negative and 50 positive authentic clinical specimens that are confirmed by certified gold-standard qPCR obtained from the SARS-CoV-2 biorepository and the CII sample repository will be analyzed. Tests will also be performed to assess potential high dose hook effect by applying increasing amounts of analyte to the biosensor up to the highest biologically observed concentrations (˜5×10⁷ TCID50/ml). Furthermore, to assess the effect of potentially inhibitory substances, common nasal drops, gels, and sprays used in the treatment of respiratory illness will be added to contrived nasal swabs for to be tested. The final diagnostic biosensor kit will also contain standard assay controls (i) a color table sheet defining negative and positive GPCR color threshold, and (ii) a positive performance control (freeze-dried target peptide that will be added to negative tests after the incubation time has elapsed to verify correct functionality of the biosensor upon contact with the target analyte).

### Example 12: Detection of Full-Length Proteins Using the Live Yeast Biosensors

This example shows that the yeast biosensors can detect full-length proteins that include the analyte of interest. Artificial fusions between α-factor and a small monomeric protein were created to directly test for protein activation. For the small monomeric protein, the yeast SUMO protein, Smt3 (MM=12 kDa), which has surface-exposed N- and C-termini was chosen. SUMO is well-behaved and well-characterized and is easily expressed and purified from Escherichia coli W. Sheng and X. Liao, “Solution structure of a yeast ubiquitin-like protein Smt3: The role of structurally less defined sequences in protein-protein recognitions,” Protein Sci., vol. 11, no. 6, pp. 1482-1491, June 2002).

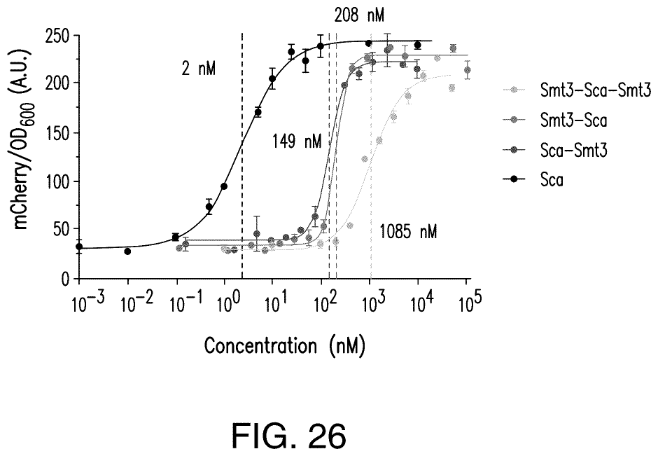

First, constructs were generated by genetically fusing α-factor to either the N- or C-terminus of the full-length Smt3 (Scα-Smt3 and Smt3-Scα, respectively) and attaching a 6×-His-tag to the opposite terminus, for Ni-NTA affinity purification (E. R. LaVallie, “Production of Recombinant Proteins in Escherichia coli,” Curr. Protoc. Protein Sci., vol. 00, no. 1, June 1995). Next, a fusion construct was developed containing two Smt3 fused together in tandem with an α-factor linker (Smt3-Scα-Smt3) and an N-terminus 6×-His-tag. All three fusion chimeras were purified from E. coli via the His-tag under standard conditions. All three chimeric α-factor proteins activated the Ste2 receptor. The EC₅₀ values for Scα-Smt3, Smt3-Scα, Smt3-Scα-Smt3 were 149 nM, 208 nM, and 1,085 nM, respectively, compared to 2 nM of Scα alone (FIG. 26). These results support the feasibility of recognizing full-length protein such as full-length coronavirus protein biomarkers with the fungal GPCRs. This result is consistent with the activation of major classes of mammalian GPCRs by full-length proteins. It is anticipated that EC₅₀ values will improve with additional rounds of directed evolution.

### Example 13: Development of a Live Yeast Biosensor for Detecting Protein Variants

This example discloses the development and validation of a live yeast-based assay for detection of protein variants in a sample.

This assay is based on the detection of a variant of the mating peptide of Saccharomyces cerevisiae, ScPep, through activation of a heterologously expressed ScSte2p GPCR in the presence of the Saccharomyces cerevisiae protease ScBar1 in Saccharomyces cerevisiae, which then transcriptionally activates the biosynthesis of fluorescent protein ymTurquoise2. This assay was used to distinguish between the two ScPep variants. Saccharomyces cerevisiae was genetically-engineered to express the ScSte2p GPCR, the ScBar1 protease and an inducible fluorescent protein that is activated upon binding of ScPep, distinguished between these two peptides (FIG. 27B). Peptide protease ScBar1, which cleaves cognate ScPep, was constitutively secreted. Peptide cleavage by the protease prevents the activation of cognate GPCR up to the protease saturation point which is observed as a shift in apparent EC₅₀ (FIG. 27C). In addition, the GPCR response to the parent and variant peptide without the protease show similar activation curve, while in the presence of the protease only one of the peptides is cleaved and its activation was pushed outside of the expected peptide concentration range (FIG. 27D). Experimentally, such activation pattern was achieved in the peptide concentration range from 100 nM to 1 μM with two ScPep variants, where the first one has alanine instead of tryptophan in the third position (WHALQLKPGQPMY) and the second one has alanine instead of glutamine in the fifth position (WHWLALKPGQPMY) (FIG. 27E).

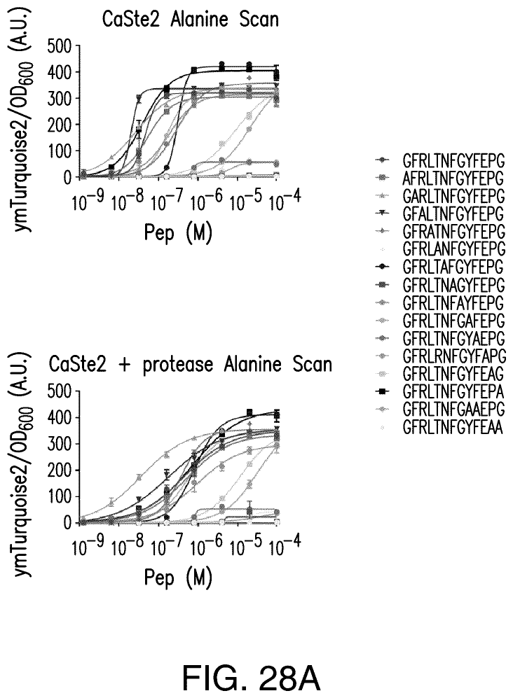

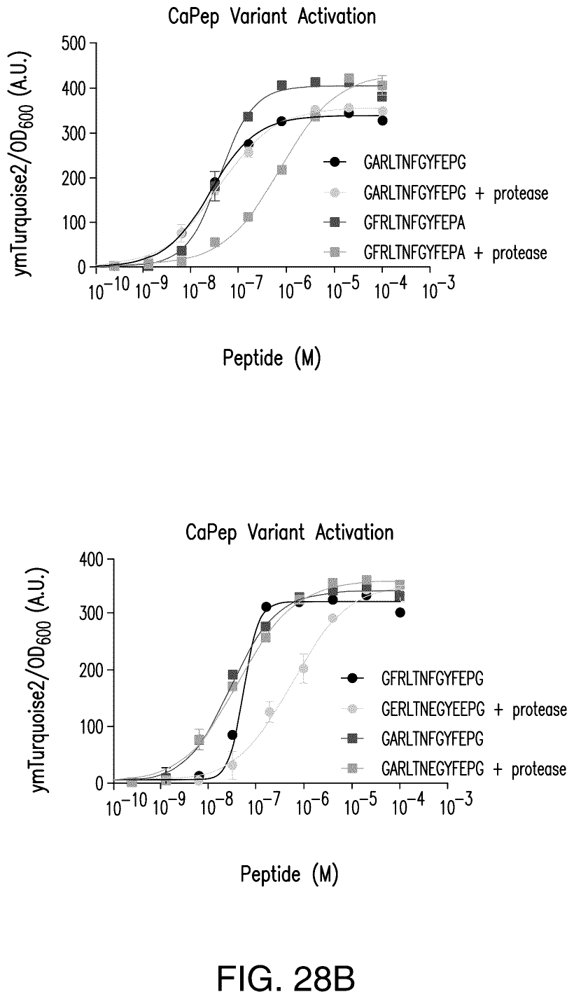

To further confirm the use of proteases to differentiate between variants, an additional assay was generated to distinguish between the parent mating peptide of Candida albicans CaPep (GFRLTNFGYFEPG) and CaPep variants. Saccharomyces cerevisiae was genetically-engineered to express the CaSte2p GPCR, the CaBar1 protease and an inducible fluorescent protein that is activated upon binding of CaPep. An alanine scan was performed in the absence and presence of CaBar1 to identify CaPep variants that bind to the CaSte2p and identify variants that are cleaved by CaBar1 (FIG. 28A). A specific CaPep variant (GARLTNFGYFEPG), which has a single amino acid substitution compared to CaPep, was selected for further analysis. As shown in FIG. 28B (right panel), the CaBar1 protease specifically cleaved the parent (wild type) sequence CaPep, resulting in the inability of the parent CaPep to bind and activate the CaSte2p GPCR. The CaBar1 protease does not cleave CaPep variant (GARLTNFGYFEPG) and this CaPep variant binds to an activates the CaSte2p GPCR in the presence of the CaBar1 protease (FIG. 28B (right panel)). An additional CaPep variant (GFRLTNFGYFEPA), which binds to the CaSte2p GPCR and has a single amino acid substitution compared to the parent CaPep, was analyzed in the absence or presence of the CaBar1 protease. As shown in FIG. 28B (left panel), the CaBar1 protease specifically cleaved the CaPep variant (GFRLTNFGYFEPA), resulting in the inability of this CaPep variant to bind and activate the CaSte2p GPCR.

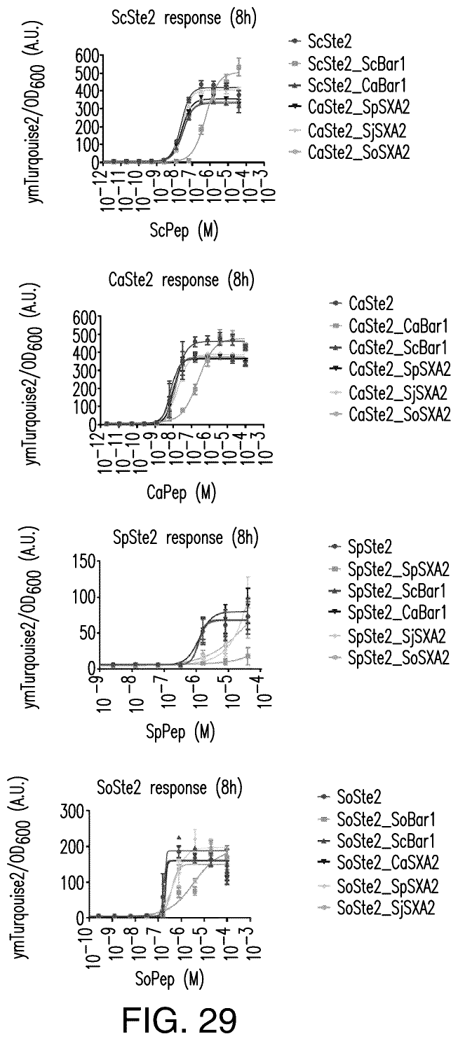

As shown in FIG. 29, fungal peptide proteases are highly specific for their cognate peptides. An apparent shift in EC₅₀ was observed most strongly only when the cognate protease is co-expressed. The live yeast-based assay disclosed herein allows the detection of protein variants in a sample by differential protease cleavage.

An exemplary paper-based dipstick protocol for performing the assays disclosed herein is provided in FIG. 27A. As shown in FIG. 27A, a dipstick can include engineered yeast patches that are responsive to the wild type (parent) and variant peptide with red pigment as a readout and either expressing (+ protease) or not expressing (− protease) specific peptide protease for peptide differentiation.

### Example 14: Development of a Live Yeast Biosensor for Detecting S Protein Variants of SARS-CoV-2

Viral variants can also be differentiated by introducing a protease that selectively cleaves either the variant or the wild type sequence in the epitope recognition motif, so that the epitope that is cleaved can no longer activate the GPCR As shown in FIG. 29, the fungal peptide/GPCR mating system has proteases that specifically cleave the opposite mating type's pheromone to create a gradient which guides the yeast towards its mating partner. These proteases can be engineered using directed evolution to selectively cleave either the variant or the wild type protein and then expressed in the biosensing yeast such that the variant or wild type epitope is sufficiently cleaved that it can no longer activate the GPCR

For the SARS-CoV-2 S protein variant that has an E484K mutation, which is the defining mutation of SARS-CoV-2 variant beta and gamma, the S. cerevisiae ScBar1 protease will be used because the native peptide of the B. compniacensis GPCR is most like the native ScBar1 peptide ligand. How the variant activates the evolved GPCR for SARS-CoV-2 will be validated to determine if it gives a similar response as the original, directed evolution of the protease can then begin.

### Example 15: Development of a Live Yeast Biosensor Product Prototype for Detecting Protein Variants of Viruses

To demonstrate the utility of biosensors as low-tech systems for the detection and differentiation of peptide variants, GPCR-based peptide-responsive yeast strains were engineered which, when coupled with the presence or absence of protease expression, allowed for visual discrimination between select peptide variants. GPCR-induced lycopene expression serves as the general detection platform whereas protease expression serves to enable discrimination between peptide variants due to its greater substrate selectivity and capacity to inhibit GPCR activation. Five fungal pheromone proteases (S. cerevisiae, C. albicans, S. pombe, S. octosporus, S. japonicus) were tested for activity with respect to both their cognate and non-cognate peptides by use of a dose response validation assay, wherein an observed shift in EC₅₀ was correlated to the effective cleavage of the peptide of interest. Tested peptide proteases were highly specific for their cognate peptides and only cross-cleaved peptides which had high sequence homology (S. pombe, S. octosporus, and S. japonicus). Furthermore, an alanine scan was used to assay both GPCR and protease activity of S. cerevisiae and C. albicans pairs with respect to their cognate peptides. Alanine scans revealed that all point mutations in S. cerevisiae peptide were tolerated by both cognate GPCR and protease. However, point mutations in C. albicans peptide had more prominent effect in GPCR activation, as certain mutations led to either complete extinction of GPCR activation or significantly altered protease activity, or both. Within this alanine scan of C. albicans, two suitable peptide variants were found which possessed similar dose-response curves to that of the non-variant peptide while also exhibiting significantly lowered protease activity with respect to the non-variant peptide. Nanomolar concentrations of these variant peptides were then visually discriminated from one another using the aforementioned live yeast biosensor assay, thus establishing a proof of concept for the use of live yeast-based biosensors as modular systems for peptide variant detection.

This example demonstrates the utility of a live yeast diagnostic that co-expresses fungal proteases to both detect and differentiate peptide variants. The yeast diagnostics were engineered with strong constitutive promoters that express the GPCRs and proteases. In practice, the target peptide induces a promoter that emit a measurable readout: either fluorescence for laboratory calibration, or a visible pigment for use at the point-of-care (FIG. 30A). For research purposes, the dose-response was measured by an EC₅₀ value (concentration of peptide required for half-maximal activation) to estimate GPCR sensitivity to the peptide. Co-expression with proteases requires a higher concentration of peptide to activate the GPCR receptor and shifts the dose-response curve and EC₅₀ (FIG. 30B). Using dose response curves, GPCR specificity toward different peptides and proteases pairs was screened for using alanine scans and noncognate pairs (FIG. 30C). This example shows the detection and confirmation of specific point mutation in target peptides through a simple color change (FIG. 30D).

### Materials and Methods

Bacterial strains and growth media: NEB® C3040 E. coli was used for all cloning experiments. Selection and growth of E. coli was performed in Luria Broth (LB) medium at 37° C. with aeration. With the exception of generating competent cells, the LB medium was supplemented with appropriate antibiotics (ampicillin 100 μg/mL, or chloramphenicol 34 μg/mL).

Yeast strains and growth media: List of used yeast strains is available in Table 12. Yeast extract peptone dextrose (YPD) was used for culturing cells: 1% (w/v) Bacto Yeast Extract (Fisher), 2% (w/v) Bacto Peptone (Fisher), 2% glucose (Fisher). Cells were cultured at 30° C. shaking at 200 rpm. Selection of yeast transformants was performed on synthetic complete (SC) dropout agar medium: 2% (w/v) glucose (Fisher), 0.67% (w/v) Yeast Nitrogen Base without amino acids (Sigma), 0.14% (w/v) Yeast Synthetic Drop-out Medium Supplements without histidine, leucine, tryptophan, and uracil (Sigma) supplemented with 20 mg/L tryptophan (Sigma), and 20 g/L bacteriological agar (Fisher). Depending on the required selection, SC dropout media was supplemented with 20 mg/L uracil (Sigma), 100 mg/L leucine (Sigma), and 20 mg/L histidine (Sigma). Cells were grown in liquid media at 30° C. with shaking. Agar was added to 2% for preparing solid yeast media which was grown static at 30° C.

All liquid experiments were performed in synthetic complete (SC) medium with 2% (w/v) glucose (VWR), 0.67% (w/v) Yeast Nitrogen Base without amino acids (Sigma), 0.14% (w/v) Yeast Synthetic Drop-out Medium Supplements without histidine, leucine, tryptophan, and uracil (Sigma), 20 mg/L uracil (Sigma), 100 mg/L leucine (Sigma), 20 mg/L histidine (Sigma), and 20 mg/mL tryptophan (Sigma) unless otherwise stated. Unless otherwise stated, all yeast strains were cultured in 200 μL of SC medium and grown in 200-μL 96-well plates at 30° C. in Glas-Col high-frequency shaker, shaking at 800 rpm.

TABLE 12

| Strain name | Genotype | Comment | Reference |
|---|---|---|---|
| yWS890 | yWS677 pCCW12-STE2- | Starting strain for optimized | [22] |
| | tSSA1-pPGK1-GPA1-tENO2- | parental strain | |
| | pRAD27-LexA-PRD-tENO1- | | |
| | URA3, LexO (6x)-pLEU2m- | | |
| | sfGFP-tTDH1-LEU2 | | |
| yTC370 | yWS890 pCCW12-STE2-tSSA1- | Optimized parental strain | This |
| | URA3 Δ, LexO (6x)-pLEU2m- | | example |
| | sfGFP-tTDH1-LEU2 Δ | | |
| yTC391 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout | |
| | tTDH1-LEU2 | | |
| yTC398 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | | |
| | tENO2-LEU2 | | |
| yTC492 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | | |
| | tENO2-LEU2 | | |
| yTC493 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and SpSxa2 protease | |
| | tTDH1-pCCW12-SpSXA2- | | |
| | tENO2-LEU2 | | |
| yTC494 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and SjSxa2 protease | |
| | tTDH1-pCCW12- SjSXA2- | | |
| | tENO2-LEU2 | | |
| yTC495 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and SoSxa2 protease | |
| | tTDH1-pCCW12- SoSXA2- | | |
| | tENO2-LEU2 | | |
| yTC384 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout | |
| | tTDH1-LEU2 | | |
| yTC399 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | | |
| | tENO2-LEU2 | | |
| yTC496 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | | |
| | tENO2-LEU2 | | |
| yTC497 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and SpSxa2 protease | |
| | tTDH1-pCCW12-SpSXA2- | | |
| | tENO2-LEU2 | | |
| yTC498 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and SjSxa2 protease | |
| | tTDH1-pCCW12- SjSXA2- | | |
| | tENO2-LEU2 | | |
| yTC499 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and SoSxa2 protease | |
| | tTDH1-pCCW12- SoSXA2- | | |
| | tENO2-LEU2 | | |
| yTC530 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout | |
| | tTDH1-LEU2 HO:: pTDH3- | | |
| | SpSTE2-tSTE2-LexO(6x)- | | |
| | pLEU2m-ymTurquoise2- | | |
| | tTDH1-HIS3 | | |
| yTC529 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout and SpSxa2 protease | |
| | tTDH1-pCCW12-SpSXA2- | | |
| | tENO2-LEU2 HO:: pTDH3- | | |
| | SpSTE2-tSTE2-LexO(6x)- | | |
| | pLEU2m-ymTurquoise2- | | |
| | tTDH1-pCCW12-SpSXA2- | | |
| | tENO2-HIS3 | | |
| yTC533 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout and SjSxa2 protease | |
| | tTDH1-pCCW12-SjSXA2- | | |
| | tENO2-LEU2 HO:: pTDH3- | | |
| | SpSTE2-tSTE2-LexO(6x)- | | |
| | pLEU2m-ymTurquoise2- | | |
| | tTDH1-pCCW12-SjSXA2- | | |
| | tENO2-HIS3 | | |
| yTC534 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout and SoSxa2 protease | |
| | tTDH1-pCCW12-SoSXA2- | | |
| | tENO2-LEU2 HO:: pTDH3- | | |
| | SpSTE2-tSTE2-LexO(6x)- | | |
| | pLEU2m-ymTurquoise2- | | |
| | tTDH1-pCCW12-SoSXA2- | | |
| | tENO2-HIS3 | | |
| yTC531 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | | |
| | tENO2-LEU2 HO:: pTDH3- | | |
| | SpSTE2-tSTE2-LexO(6x)- | | |
| | pLEU2m-ymTurquoise2- | | |
| | tTDH1-pCCW12-ScBAR1- | | |
| | tENO2-HIS3 | | |
| yTC532 | yTC370 ARS208a:: pTDH3- | Integrated SpSte2 GPCR with | This |
| | SpSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | | |
| | tENO2-LEU2 HO:: pTDH3- | | |
| | SpSTE2-tSTE2-LexO(6x)- | | |
| | pLEU2m-ymTurquoise2- | | |
| | tTDH1-pCCW12-CaBAR1- | | |
| | tENO2-HIS3 | | |
| yTC404 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout | |
| | tTDH1-LEU2 | | |
| yTC401 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout and SjSxa2 protease | |
| | tTDH1-pCCW12-SjSXA2- | | |
| | tENO2-LEU2 | | |
| yTC507 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout and SoSxa2 protease | |
| | tTDH1-pCCW12-SoSXA2- | | |
| | tENO2-LEU2 | | |
| yTC504 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | | |
| | tENO2-LEU2 | | |
| yTC505 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | | |
| | tENO2-LEU2 | | |
| yTC506 | yTC370 ARS208a:: pTDH3- | Integrated SjSte2 GPCR with | This |
| | SjSTE2-tSTE2-LexO(6x)- | ymTurquoise2 fluorescent | example |
| | pLEU2m-ymTurquoise2- | readout and SpSxa2 protease | |
| | TDH1-pCCW12-SpSXA2- | | |
| | tENO2-LEU2 | | |
| yTC405 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout | |
| | tTDH1-LEU2 | | |
| yTC402 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout and SoSxa2 protease | |
| | tTDH1-pCCW12-SoSXA2- | | |
| | tENO2-LEU2 | | |
| yTC508 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | | |
| | tENO2-LEU2 | | |
| yTC509 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | | |
| | tENO2-LEU2 | | |
| yTC510 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout and SpSxa2 protease | |
| | tTDH1-pCCW12-SpSXA2- | | |
| | tENO2-LEU2 | | |
| yTC511 | yTC370 ARS208a:: pTDH3- | Integrated SoSte2 GPCR with | This |
| | SoSTE2-tSTE2-LexO(6x)- | ymTurquoise2 fluorescent | example |
| | pLEU2m-ymTurquoise2- | readout and SjSxa2 protease | |
| | tTDH1-pCCW12-SjSXA2- | | |
| | tENO2-LEU2 | | |
| yBB01 | yTC370 ARS208a:: pTDH3- | Integrated ScSte2 GPCR with | This |
| | ScSTE2-tSTE2-LexO(6x)- | ymTurquoise2 fluorescent | example |
| | pLEU2m-ymTurquoise2- | readout and ScBar1 protease | |
| | tTDH1-pCCW12-ScBAR1- | (two copies) | |
| | tENO2-LEU2 HO:: pCCW12- | | |
| | ScBAR1-tENO2-HIS3 | | |
| yBB02 | yTC370 ARS208a:: pTDH3- | Integrated CaSte2 GPCR with | This |
| | CaSTE2-tSTE2-LexO(6x)- | ymTurquoise2 fluorescent | example |
| | pLEU2m-ymTurquoise2- | readout and CaBar1 protease | |
| | tTDH1-pCCW12-CaBAR1- | (two copies) | |
| | tENO2-LEU2 HO:: pCCW12- | | |
| | CaBAR1-tENO2-HIS3 | | |
| yTC412 | yTC370 ARS208a:: pTDH3- | Receptor-less lycopene strain | This |
| | FAD1-tPGK1-LexO(6x)- | with two pheromone-inducible | example |
| | pLEU2m-CrtI-tACT1-LEU2 | copies of CrtI | |
| | HO:: pTEF1-CrtE-tADH1- | | |
| | CrtB-pPGK1-LexO(6x)- | | |
| | pLEU2m-CrtI-tACT1-HIS3 | | |
| yTC681 | yTC370 ARS208a:: pTDH3- | Receptor-less lycopene strain | This |
| | FAD1-tPGK1-LexO(6x)- | with two pheromone-inducible | example |
| | pLEU2m-CrtI-tACT1-pCCW12- | copies of CrtI and constitutive | |
| | CaBAR1-tENO2-LEU2 HO:: | CaBar1 protease | |
| | pTEF1-CrtE-tADH1-CrtB- | | |
| | pPGK1-LexO(6x)-pLEU2m- | | |
| | CrtI-tACT1-HIS3 | | |
| yTC646 | yTC412 LEU2:: pTDH3- | CaSte2 lycopene biosensor | This |
| | CaSTE2-tSTE2-URA3 | | example |
| yTC682 | yTC681 LEU2:: pTDH3- | CaSte2 + CaBar1 lycopene | This |
| | CaSTE2-tSTE2-pCCW12- | biosensor | example |
| | CaBAR1-tENO2-URA3 | | |

Materials: Synthetic peptides (≥95% purity) were obtained from GenScript (Piscataway, NJ, USA). S. cerevisiae alpha-factor (ScPep) was obtained from Zymo Research (Irvine, CA, USA). Stock synthetic peptide solutions were prepared by resuspending peptide powder in sterile MiliQ H₂O. Polymerases, restriction enzymes, T4 ligase, Gibson assembly mix and appropriate buffers were obtained from New England Biolabs (NEB) (Ipswich, MA, USA). Primers and synthetic DNA (gBlocks) were obtained from Integrated DNA Technologies (IDT, Coralville, Iowa, USA) and resuspended in MiliQ H₂O. Plasmids were cloned and amplified in E. coli C3040 (NEB). 96-well microtiter plates were obtained from Corning (Corning Inc.).

Bacterial transformations: Electrocompetent cells were created as follows. C3040 E. coli obtained from New England Biolabs (NEB) (Ipswitch, MA, USA) from the −80° C. glycerol stock was streaked out on LB plate and grown overnight. A single colony was picked and grown for 14-16 hours in 2 mL of LB medium. Then the whole culture was added to 2 L of low-salt SOB containing 2% (w/v) tryptone, 0.5% (w/v) Bacto Yeast Extract and 0.05% (w/v) sodium chloride. The culture was grown for 5-6 h to OD₆₀₀˜0.6-0.8, split between four 500 mL centrifuge-safe bottles, and centrifuged at 6,000 rpm at 4° C. for 10 min. The supernatant was then discarded, and the cell pellets resuspended by aspiration in ice-cold 10% (v/v) glycerol. The cells were pelleted again, the supernatant was discarded, the cells were resuspended in glycerol, unified in two bottles, and centrifuged 6,300 rpm at 4° C. for 15 min. Previous steps were repeated two more times with final unification in a single bottle and final resuspension in 3 mL of ice-cold 10% (v/v) glycerol. 100 μL of the cell suspension was aliquoted into 1.5-mL Eppendorf tubes, flash frozen on dry ice, and put into −80° C. freezer for long term storage. To transform the DNA, 50 μL of the electrocompetent cells were thawed on ice and 5 μL of 1:3 water dilution of DNA was added to the cells. Cell/DNA mixture was transferred to 0.1-cm gap electroporation cuvette (Bio-Rad) and one 1.8 kV pulse was given. The cells were recovered in 1 mL LB medium at 37° C. for 30-45 min. Cells were then plated on solid LB medium supplemented with the appropriate antibiotics.

Yeast transformations: Chemically competent yeast cells were created following the lithium acetate method [16]. Yeast colonies were grown to saturation overnight in YPD. The following morning the cells were diluted 1:100 in 5 mL of fresh YPD in a 15-mL culture tube and grown for 4-6 h to OD₆₀₀ 0.8-1.0. Cells were pelleted and washed once with 5 mL 0.1 M lithium acetate (LiOAc) (Sigma). Cells were then resuspended in 0.1 M LiOAc to a total volume of 100 μL/transformation. 100 μL of cell suspension was then distributed into 1.5 mL reaction tubes and pelleted. Cells were resuspended in 64 μL of DNA/salmon sperm DNA mixture (10 μL of boiled salmon sperm DNA (Fisher)+DNA+ddH₂O), and then mixed with 294 μL of PEG/LiOAc mixture (260 μL 50% (w/v) PEG-3350 (Sigma)+36 μL 1 M LiOAc). The yeast transformation mixture was then heat-shocked at 42° C. for 40 mins, pelleted, resuspended in 150 μL 5 mM CaCl₂) (Sigma) and plated onto the appropriate synthetic dropout medium.

Construction of communication cassette plasmids and yeast genomic integration plasmids: List of all used plasmids is available in Table 13. List of expression modules and part sequences is available in Table 14. The communication cassette plasmids are based on pYTK009 (ori, CmR selection marker) from MoClo Yeast Toolkit [17]. Cassette plasmids having individual expression cassettes with biosensor parts (constitutive GPCR, peptide-inducible readout or constitutive protease) were cloned using Gibson Assembly and further on matched using Golden Gate Assembly (GGA). Yeast genomic integration plasmid is an acceptor plasmid (ori, AmpR selection marker) into which biosensor component cassette plasmids are being assembled via GGA. The integration plasmid contains appropriate 500-bp homology sequences for genomic integration (ARS208a, HO or LEU2), integration selection marker (LEU2, HIS3 or URA3), red fluorescent protein expression cassette which gets replaced with correctly assembled biosensor parts and unique NotI restriction sites for repair fragment linearization.

TABLE 13

| Name | Construct details | Selection | Comments |
|---|---|---|---|
| pTC160 | Cas9-gRNA (STE2) | Amp, | CRISPR genomic integration |
| | | NAT | |
| pTC200 | Cas9-gRNA (URA3) | Amp, | CRISPR genomic integration |
| | | NAT | |
| pTC220 | Cas9-gRNA (LEU2) | Amp, U- | CRISPR genomic integration |
| pTC11 | Cas9-gRNA (ARS208a) | Amp, U- | CRISPR genomic integration |
| pTC12 | Cas9-gRNA (HO) | Amp, U- | CRISPR genomic integration |
| pTC393 | LEU2_CAGT_RFP_TTTT_URA3 | Amp | Acceptor plasmid in GGA |
| pTC197 | ARS208a_CAGT_RFP_TTTT_LEU2 | Amp | Acceptor plasmid in GGA |
| pTC199 | HO_CAGT_RFP_TTTT_HIS3 | Amp | Acceptor plasmid in GGA |
| pTC394 | Cas9-gRNA (LEU2) | Amp, | CRISPR genomic integration |
| | | NAT | |
| pTC223 | CAGT-pGPD_CaSTE2_tSTE2-CCAA | Cam | GPCR cassette plasmid |
| pTC230 | CAGT-pGPD_ScSTE2_tSTE2-CCAA | Cam | GPCR cassette plasmid |
| pTC231 | CAGT-pGPD_SjSTE2_tSTE2-CCAA | Cam | GPCR cassette plasmid |
| pTC232 | CAGT-pGPD_SoSTE2_tSTE2-CCAA | Cam | GPCR cassette plasmid |
| pTC233 | CAGT-pGPD_SpSTE2_tSTE2-CCAA | Cam | GPCR cassette plasmid |
| pTC239 | CCAA-LexO(6x)-pLEU2m- | Cam | Fluorescent readout cassette |
| | ymTurquoise2 tTDH1-GCAT | | plasmid |
| pTC262 | GAGT-pCCW12_ScBAR1_tENO2- | Cam | Protease cassette plasmid |
| | TTTT | | |
| pTC263 | GAGT-pCCW12_CaBAR1_tENO2- | Cam | Protease cassette plasmid |
| | TTTT | | |
| pTC264 | GAGT-pCCW12_SpSXA2_tENO2- | Cam | Protease cassette plasmid |
| | TTTT | | |
| pTC265 | GAGT-pCCW12_SjSXA2_tENO2- | Cam | Protease cassette plasmid |
| | TTTT | | |
| pTC266 | GAGT-pCCW12_SoSXA2_tENO2- | Cam | Protease cassette plasmid |
| | TTTT | | |
| pTC288 | CCAA_GCAT | Cam | Connector cassette plasmid |
| pTC289 | GCAT_GAGT | Cam | Connector cassette plasmid |
| pTC290 | GAGT_TTTT | Cam | Connector cassette plasmid |
| pTC291 | GCAT_TTTT | Cam | Connector cassette plasmid |
| pTC274 | ARS208a_CaSTE2_ymTurquoise2_LEU2 | Amp | Complete integration plasmid |
| pTC281 | ARS208a_ScSTE2_ymTurquoise2_LEU2 | Amp | Complete integration plasmid |
| pTC292 | ARS208a_ScSTE2_ymTurquoise2_ScBAR1_LEU2 | Amp | Complete integration plasmid |
| pTC293 | ARS208a_CaSTE2_ymTurquoise2_CaBAR1_LEU2 | Amp | Complete integration plasmid |
| pTC294 | ARS208a_SpSTE2_ymTurquoise2_SpSXA2_LEU2 | Amp | Complete integration plasmid |
| pTC295 | ARS208a_SjSTE2_ymTurquoise2_SjSXA2_LEU2 | Amp | Complete integration plasmid |
| pTC296 | ARS208a_SoSTE2_ymTurquoise2_SoSXA2_LEU2 | Amp | Complete integration plasmid |
| pTC297 | ARS208a_SpSTE2_ymTurquoise2_LEU2 | Amp | Complete integration plasmid |
| pTC298 | ARS208a_SjSTE2_ymTurquoise2_LEU2 | Amp | Complete integration plasmid |
| pTC299 | ARS208a_SoSTE2_ymTurquoise2_LEU2 | Amp | Complete integration plasmid |
| pTC300 | HO_CrtE_CrtB_LexO-CrtI_HIS3 | Amp | Complete integration plasmid |
| pTC301 | ARS208a_FAD1_LexO-CrtI_LEU2 | Amp | Complete integration plasmid |
| pTC335 | ARS208a_ScSTE2_ymTurquoise2_CaBAR1_LEU2 | Amp | Complete integration plasmid |
| pTC336 | ARS208a_ScSTE2_ymTurquoise2_SpSXA2_LEU2 | Amp | Complete integration plasmid |
| pTC337 | ARS208a_ScSTE2_ymTurquoise2_SjSXA2_LEU2 | Amp | Complete integration plasmid |
| pTC338 | ARS208a_ScSTE2_ymTurquoise2_SoSXA2_LEU2 | Amp | Complete integration plasmid |
| pTC339 | ARS208a_CaSTE2_ymTurquoise2_ScBAR1_LEU2 | Amp | Complete integration plasmid |
| pTC340 | ARS208a_CaSTE2_ymTurquoise2_SpSXA2_LEU2 | Amp | Complete integration plasmid |
| pTC341 | ARS208a_CaSTE2_ymTurquoise2_SjSXA2_LEU2 | Amp | Complete integration plasmid |
| pTC342 | ARS208a_CaSTE2_ymTurquoise2_SoSXA2_LEU2 | Amp | Complete integration plasmid |
| pTC343 | ARS208a_SpSTE2_ymTurquoise2_ScBAR1_LEU2 | Amp | Complete integration plasmid |
| pTC344 | ARS208a_SpSTE2_ymTurquoise2_CaBAR1_LEU2 | Amp | Complete integration plasmid |
| pTC345 | ARS208a_SpSTE2_ymTurquoise2_SjSXA2_LEU2 | Amp | Complete integration plasmid |
| pTC346 | ARS208a_SpSTE2_ymTurquoise2_SoSXA2_LEU2 | Amp | Complete integration plasmid |
| pTC347 | ARS208a_SjSTE2_ymTurquoise2_ScBAR1_LEU2 | Amp | Complete integration plasmid |
| pTC348 | ARS208a_SjSTE2_ymTurquoise2_CaBAR1_LEU2 | Amp | Complete integration plasmid |
| pTC349 | ARS208a_SjSTE2_ymTurquoise2_SpSXA2_LEU2 | Amp | Complete integration plasmid |
| pTC350 | ARS208a_SjSTE2_ymTurquoise2_SoSXA2_LEU2 | Amp | Complete integration plasmid |
| pTC351 | ARS208a_SoSTE2_ymTurquoise2_ScBAR1_LEU2 | Amp | Complete integration plasmid |
| pTC352 | ARS208a_SoSTE2_ymTurquoise2_CaBAR1_LEU2 | Amp | Complete integration plasmid |
| pTC353 | ARS208a_SoSTE2_ymTurquoise2_SpSXA2_LEU2 | Amp | Complete integration plasmid |
| pTC354 | ARS208a_SoSTE2_ymTurquoise2_SjSXA2_LEU2 | Amp | Complete integration plasmid |
| pTC368 | HO_SpSTE2_ymTurquoise2_SpSXA2_HIS3 | Amp | Complete integration plasmid |
| pTC369 | HO_SpSTE2_ymTurquoise2_HIS3 | Amp | Complete integration plasmid |
| pTC420 | HO_SpSTE2_ymTurquoise2_ScBAR1_HIS3 | Amp | Complete integration plasmid |
| pTC421 | HO_SpSTE2_ymTurquoise2_CaBAR1_HIS3 | Amp | Complete integration plasmid |
| pTC422 | HO_SpSTE2_ymTurquoise2_SjSXA2_HIS3 | Amp | Complete integration plasmid |
| pTC423 | HO_SpSTE2_ymTurquoise2_SoSXA2_HIS3 | Amp | Complete integration plasmid |
| pBB01 | HO_ScBAR1_HIS3 | Amp | Complete integration plasmid |
| pBB02 | HO_CaBAR1_HIS3 | Amp | Complete integration plasmid |
| pTC546 | LEU2_CaSTE2_CaBAR1_URA3 | Amp | Complete integration plasmid |
| pTC549 | LEU2_CaSTE2_URA3 | Amp | Complete integration plasmid |
| pTC561 | ARS208a_FAD1_LexO_CrtI_CaBAR1_LEU2 | Amp | Complete integration plasmid |

TABLE 14

| | |
|---|---|
| Description | Sequence |

GPCR part sequence

| | |
|---|---|
| pTDH3- | GGTCTCACAGTAGTTTATCATTATCAATACTGCCATTTCAAAGAATACGTAAATA |
| GPCR- | ATTAATAGTAGTGATTTTCCTAACTTTATTTAGTCAAAAAATTAGCCTTTTAATT |
| tSTE2 | CTGCTGTAACCCGTACATGCCCAAAATAGGGGGCGGGTTACACAGAATATATAAC |
| | ATCGTAGGTGTCTGGGTGAACAGTTTATTCCTGGCATCCACTAAATATAATGGAG |
| | CCCGCTTTTTAAGCTGGCATCCAGAAAAAAAAAGAATCCCAGCACCAAAATATTG |
| | TTTTCTTCACCAACCATCAGTTCATAGGTCCATTCTCTTAGCGCAACTACAGAGA |
| | ACAGGGGCACAAACAGGCAAAAAACGGGCACAACCTCAATGGAGTGATGCAACCT |
| | GCCTGGAGTAAATGATGACACAAGGCAATTGACCCACGCATGTATCTATCTCATT |
| | TTCTTACACCTTCTATTACCTTCTGCTCTCTCTGATTTGGAAAAAGCTGAAAAAA |
| | AAGGTTGAAACCAGTTCCCTGAAATTATTCCCCTACTTGACTAATAAGTATATAA |
| | AGACGGTAGGTATTGATTGTAATTCTGTAAATCTATTTCTTAAACTTCTTAAATT |
| | CTACTTTTATAGTTAGTCTTTTTTTTAGTTTTAAAACACCAAGAACTTAGTTTCG |
| | ACGGATACTAGTAAA[receptor |
| | ORF]CTCGAGACGGCTTTGAAAAAGTAATTTCGTGACCTTCGGTATAAGGTTACT |
| | ACTAGATTCAGGTGCTCATCAGATGCACCACATTCTCTATAAAAAAAAATGGTAT |
| | CTTTCTTATTTGATAATATTTAAACTCCTTTACATAATAAACATCTCGTAAGTAG |
| | TGGTAGAAACCACCTTTGCTTTTACGAGTTCACCAACGAGACC |

Readout part sequence

| | |
|---|---|
| LexO(6x)- | GGTCTCACCAAAAACGGGTAGTCCATCGTTGTAGGATACTGTATATACACCCAGT |
| pLEU2m- | AGAGTAGGTGACTACTGTATGAGCATACAGTAGTGACAACCACTTACTGTATATA |
| readout- | AATACAGTAGTGGTCATCGATACTGTATATAAAACCAGTAGGCAATCGTTTGTAC |
| tTDH1 | TGTATGTACATACAGTATACCTCGCAACTACTGTATATAAACACAGTAATTAACT |
| | TGTAATATTCTAATCAATTGACAATATTATTTAAGGACCTATTGTTTTTTCCAAT |
| | AGGTGGTTAGCAATCGTCTTACTTTCTAACTTTTCTTACCTTTTACATTTCAGCA |
| | ATATATATATATATATTTCAAGGATATACCATTCTAAGATCTG[readout |
| | ORF]CTAACTCGAGATAAAGCAATCTTGATGAGGATAATGATTTTTTTTTGAATA |
| | TACATAAATACTACCGTTTTTCTGCTAGATTTTGTGATGACGTAAATAAGTACAT |
| | ATTACTTTTTAAGCCAAGACAAGATTAAGCATTAACTTTACCCTTTTCTTTCTAA |
| | GTTTCAATATTAGTTATCACTGTTTAAAAGTTATGGCGAGAACGTCGGCGGTTAA |
| | AATATATTACCCTGAACGGCATTGAGACC |

Peptide protease part sequence

| | |
|---|---|
| pCCW12- | GGTCTCAGAGTCACCCATGAACCACACGGTTAGTCCAAAAGGGGCAGTTCAGATT |
| protease- | CCAGATGCGGGAATTAGCTTGCTGCCACCCTCACCTCACTAACGCTGCGGTGTGC |
| tENO2 | GGATACTTCATGCTATTTATAGACGCGCGTGTCGGAATCAGCACGCGCAAGAACC |
| | AAATGGGAAAATCGGAATGGGTCCAGAACTGCTTTGAGTGCTGGCTATTGGCGTC |
| | TGATTTCCGTTTTGGGAATCCTTTGCCGCGCGCCCCTCTCAAAACTCCGCACAAG |
| | TCCCAGAAAGCGGGAAAGAAATAAAACGCCACCAAAAAAAAAAAAATAAAAGCCA |
| | ATCCTCGAAGCGTGGGTGGTAGGCCCTGGATTATCCCGTACAAGTATTTCTCAGG |
| | AGTAAAAAAACCGTTTGTTTTGGAATTTCCCATTTCGCGGCCACCTACGCCGCTA |
| | TCTTTGCAACAACTATCTGCGATAACTCAGCAAATTTTGCATATTCGTGTTGCAG |
| | TATTGCGATAATGGGAGTCTTACTTCCAACATAACGGCAGAAAGAAATGTGAGAA |
| | AATTTTGCATCCTTTGCCTCCGTTCAAGTATATAAAGTCGGCATGCTTGATAATC |
| | TTTCTTTCCATCCTACATTGTTCTAATTATTCTTATTCTCCTTTATTCTTTCCTA |
| | ACATACCAAGAAATTAATCTTCTGTCATTCGCTTAAACACTATATCAATAAAGAT |
| | CACTAGT [protease |
| | ORF]ACTAGTTAACTCGAGAGTGCTTTTAACTAAGAATTATTAGTCTTTTCTGCT |
| | TATTTTTTCATCATAGTTTAGAACACTTTATATTAACGAATAGTTTATGAATCTA |
| | TTTAGGTTTAAAAATTGATACAGTTTTATAAGTTACTTTTTCAAAGACTCGTGCT |
| | GTCTATTGCATAATGCACTGGAAGGGGAAAAAAAAGGTGCACACGCGTGGCTTTT |
| | TCTTGAATTTGCAGTTTGAAAAATTTTTTGAGACC |

Connecting part sequences

| | |
|---|---|
| CCAA-GCAT | GGTCTCACCAAGCGCCGGGCGCATTGAGACC |
| GCAT-GAGT | GGTCTCAGCATGCGCCGGGCGAGTTGAGACC |
| GAGT-TTTT | GGTCTCAGAGTGCGCCGGGCTTTTTGAGACC |
| GCAT-TTTT | GGTCTCAGCATGCGCCGGGCTTTTTGAGACC |

Lycopene part sequences

| | |
|---|---|
| pTEF1- | GGTCTCACAGTATAGCTTCAAAATGTTTCTACTCCTTTTTTACTCTTCCAGATTT |
| CrtE-tADH1- | TCTCGGACTCCGCGCATCGCCGTACCACTTCAAAACACCCAAGCACAGCATACTA |
| (CrtB-pPGK1, | AATTTCCCCTCTTTCTTCCTCTAGGGTGTCGTTAATTACCCGTACTAAAGGTTTG |
| rev), | GAAAAGAAAAAAGAGACCGCCTCGTTTCTTTTTCTTCGTCGAAAAAGGCAATAAA |
| LexO(6x)- | AATTTTTATCACGTTTCTTTTTCTTGAAAATTTTTTTTTTTGATTTTTTTCTCTT |
| pLEU2m- | TCGATGACCTCCCATTGATATTTAAGTTAATAAACGGTCTTCAATTTCTCAAGTT |
| CrtI-tACT1 | TCAGTTTCATTTTTCTTGTTCTATTACAACTTTTTTTACTTCTTGCTCATTAGAA |
| | AGAAAGCATAGCAATCTAATCTAAGTTTTAATTACAAAatggtttctggttcgaa |
| | agcaggagtatcacctcatagggaaatcgaagtcatgagacagtccattgatgac |
| | cacttagcaggattgttgccagaaacagattcccaggatatcgttagccttgcta |
| | tgagagaaggtgttatggcacctggtaaacgtatcagacctttgctgatgttact |
| | tgctgcaagGgacctgagatatcagggttctatgcctacactactggatctagct |
| | tgtgctgttgaactgacacatactgcttccttgatgctggatgacatgccttgta |
| | tggacaatgcggaacttagaagaggtcaaccaacaacccacaagaaattcggaga |
| | atctgttgccattttggcttctgtaggtctgttgtcgaaagcttttggcttgatt |
| | gctgcaactggtgatcttccaggtgaaaggagagcacaagctgtaaacgagctat |
| | ctactgcagttggtgttcaaggtctagtcttaggacagttcagagatttgaatga |
| | cgcagctttggacagaactcctgatgctatcctgtctacgaaccatctgaagact |
| | ggcatcttgttctcagctatgttgcaaatcgtagccattgcttctgcttcttcac |
| | catctactagggaaacgttacacgcattcgcattggactttggtcaagcctttca |
| | actgctagacgatttgagggatgatcatccagagacaggtaaagaccgtaacaaa |
| | gacgctggtaaaagcactctagtcaacagattgggtgctgatgcagctagacaga |
| | aactgagagagcacattgactctgctgacaaacacctgacatttgcatgtccaca |
| | aggaggtgctataaggcagtttatgcacctatggtttggacaccatcttgctgat |
| | tggtcCccagtgatgaagatcgcctaaGCGAATTTCTTATGATTTATGATTTTTA |
| | TTATTAAATAAGTTATAAAAAAAATAAGTGTATACAAATTTTAAAGTGACTCTTA |
| | GGTTTTAAAACGAAAATTCTTATTCTTGAGTAACTCTTTCCTGTAGGTCAGGTTG |
| | CTTTCTCAGGTATAGCATGAGGTCGCTCttagacaggtctttgccataaaccagc |
| | aggtcttggtgtaactctggttgttttggcacgaatgacttgtcctggtgcagcc |
| | ataagcatcgcaatcttctctcctttgctagtgtgttgacgtctatcccaagcag |
| | aaccacctgcagcttttaccttgatgccaatctctctgtagacagatcttgcagt |
| | agctatagcccaagcacatctaggtggtagatcatgcaatccagcttgacttgag |
| | atgtaatagggttcagcagcgtctatcagcctttcagcaactcttgctaatgcag |
| | ctctgttctctcttgcagcgtaattctcaggagttagaccagcatcttgcaacca |
| | ttcagcaggtagatagcatctgtcaatagctgcatcgtcgataatatctctcgcg |
| | atgtttgtcagctgaaaagccaaacctagatcacaagctctgtccaaaacccttt |
| | cgtctcttacacccattactcttgccatcatcaaaccaactactccagcaacatg |
| | gtagcagtatctcaaggtgtcttcaaaggtcacgtaacgagtttgagcaacatcc |
| | attgcgaaaccatccaagtgatcaagtgccattcttggcgtaataccgtgtgtta |
| | gtgcaacttcttggaatgcagcaaaagcaggatcttgcatctcagcaccttcaaa |
| | agctgcaagtgtaagcgttctcaatctagccaatctctgagtagcctcttcttct |
| | gcagcagcttcagatgcgaaaccatgtgtctggtcatctataacgtcatcacagt |
| | gtctacaccaagtgtacagcataagcactgatcttctagtagctgggtcaaacag |
| | tttagctgctgtagcaaaggacttggaaccattagccatcgtttgagtagcatga |
| | tccaacaaaggtggttgactcatTGTTTTATATTTGTTGTAAAAAGTAGATAATT |
| | ACTTCCTTGATGATCTGTAAAAAAGAGAAAAAGAAAGCATCTAAGAACTTGAAAA |
| | ACTACGAATTAGAAAAGACCAAATATGTATTTCTTGCATTGACCAATTTATGCAA |
| | GTTTATATATATGTAAATGTAAGTTTCACGAGGTTCTACTAAACTAAACCACCCC |
| | CTTGGTTAGAAGAAAAGAGTGTGTGAGAACAGGCTGTTGTTGTCACACGATTCGG |
| | ACAATTCTGTTTGAAAGAGAGAGAGTAACAGTACGATCGAACGAACTTTGCTCTG |
| | GAGATCACAGTGGGCATCATAGCATGTGGTACTAAACCCTTTCCCGCCATTCCAG |
| | AACCTTCGATTGCTTGTTACAAAACCTGTGAGCCGTCGCTAGGACCTTGTTGTGT |
| | GACGAAATTGGAAGCTGCAATCAATAGGAAGACAGGAAGTCGAGCGTGTCTGGGT |
| | TTTTTCAGTTTTGTTCTTTTTGCAAACAAATCACGAGCGACGGTAATTTCTTTCT |
| | CGATAAGAGGCCACGTGCTTTATGAGGGTAACATCAATTCAAGAAGGAGGGAAAC |
| | ACTTCCTTTTTCTGGCCCTGATAATAGTATGAGGGTGAAGCCAAAATAAAGGATT |
| | CGCGCCCAAATCGGCATCTTTAAATGCAGGTATGCGATAGTTCCTCACTCTTTCC |
| | TTACTCACGAGTAATTCTTGCAAATGCCTATTATGCAGATGTTATAATATCTGTG |
| | CGTCTTGAGTTGAAGTCAGGAATCTAAAATAAAAATTAAGGTTAATAAAAAGAGG |
| | AAAGAAAAAAAAATTAATCGATTTACAGAAACTTGCACACTAAAAATACACAACT |
| | AAAAGCAATTACAGTATGGGAAGTCATCGACGTTATCTCTACTATAGTATATTAT |
| | CATTTCTATTATTATCCTGCTCAGTGGTACTTGCAAAACAAGATAAGACCCCATT |
| | CTTTGAAGGTACTTCTTCGAAAAATTCGCGTCTCCCCAAACGGGTAGTCCATCGT |
| | TGTAGGATACTGTATATACACCCAGTAGAGTAGGTGACTACTGTATGAGCATACA |
| | GTAGTGACAACCACTTACTGTATATAAATACAGTAGTGGTCATCGATACTGTATA |
| | TAAAACCAGTAGGCAATCGTTTGTACTGTATGTACATACAGTATACCTCGCAACT |
| | ACTGTATATAAACACAGTAATTAACTTGTAATATTCTAATCAATTGACAATATTA |
| | TTTAAGGACCTATTGTTTTTTCCAATAGGTGGTTAGCAATCGTCTTACTTTCTAA |
| | CTTTTCTTACCTTTTACATTTCAGCAATATATATATATATATTTCAAGGATATAC |
| | CATTCTAAGATCTAAAAAAatgaagaaaaccgtagtgattggtgcaggttttggt |
| | ggtttagctttggctatacgtctacaagctgcaggtattcctacagtgctattgg |
| | agcaaagagacaaaccaggaggaagagcttatgtttggcacgatcaaggctttac |
| | ctttgatgctggtcctacagtcatcactgatcctactgcattggaagctttgttc |
| | accttagctggtagaagaatggaagattatgtccgtctattgcctgtcaagccgt |
| | tttacagattgtgttgggaatctggtaaaaccctagattacgccaatgacagtgc |
| | tgaactagaagctcagattacgcagtttaatcccagagatgtcgaaggttacagg |
| | agattccttgcctattcccaagctgttttccaagagggttatcttcgtttgggtt |
| | cagttccattcctgtcctttagggatatgcttagagcaggtcctcagttgttgaa |
| | gctacaagcatggcaaagtgtgtatcagtctgtttcgagatttatcgaggatgaa |
| | catctgagacaagcattctcattccacagtcttctagttggaggtaatcccttta |
| | ccacatcgagcatatatacgttgattcacgctttggaaagagaatggggagtttg |
| | gtttcctgaaggtggaacaggtgctttggttaatggtatggtgaagctattcacg |
| | gatttgggtggagaaatagagctgaatgcaagagtggaagaacttgttgtagcag |
| | acaacagagtctcacaagttagacttgctgatggtaggatcttcgatacagatgc |
| | tgtagcttcaaacgcagatgtagtgaacacttataaaaagttgttgggacatcat |
| | cctgttggacaaaagagagcagctgctttggagaggaaatctatgagcaactcgt |
| | tgtttgtcctttactttgggctgaatcaaccacactcacaactagctcatcacac |
| | aatctgctttggtcctagatacagagagctgatagatgaaattttcactggatct |
| | gctttagcagacgatttttccctgtacttgcattcaccatgtgttactgatccct |
| | ctttagcaccacctggttgtgctagcttctatgtactagcacctgtaccacattt |
| | gggtaatgctccattagattgggcacaagaaggaccgaaattgagggataggatc |
| | ttcgactatttggaagaacgttacatgccaggtttgagatctcagttggttacac |
| | agaggatattcacaccagctgattttcatgatactctagatgcgcatttaggtag |
| | cgctttttccattgagccacttttgacgcaaagtgcttggtttagaccacacaac |
| | agagattctgacattgccaatctgtacctagtaggtgcaggaactcatccaggag |
| | ctggtattcctggagttgtagcttctgctaaagctactgctagtctgatgatcga |
| | ggatttgcagtaaTCTCTGCTTTTGTGCGCGTATGTTTATGTATGTACCTCTCTC |
| | TCTATTTCTATTTTTAAACCACCCTCTCAATAAAATAAAAATAATAAAGTATTTT |
| | TAAGGAAAAGACGTGTTTAAGCACTGACTTTATCTACTTTTTGTACGTTTTCATT |
| | GATATAATGTGTTTTGTCTCTCCCTTTTCTACGAAAATTTCAAAAATTGACCAAA |
| | AAAAGGAATATATATACGAAAAACTATTATATTTATATATCATAGTGTTTTTCGA |
| | GACC |
| pTDH3- | GGTCTCACAGTAGTTTATCATTATCAATACTCGCCATTTCAAAGAATACGTAAAT |
| FAD1-tPGK1, | AATTAATAGTAGTGATTTTCCTAACTTTATTTAGTCAAAAAATTAGCCTTTTAAT |
| LexO(6x)- | TCTGCTGTAACCCGTACATGCCCAAAATAGGGGGCGGGTTACACAGAATATATAA |
| pLEU2m- | CATCGTAGGTGTCTGGGTGAACAGTTTATTCCTGGCATCCACTAAATATAATGGA |
| CrtI-tACT1- | GCCCGCTTTTTAAGCTGGCATCCAGAAAAAAAAAGAATCCCAGCACCAAAATATT |
| [pCCW12- | GTTTTCTTCACCAACCATCAGTTCATAGGTCCATTCTCTTAGCGCAACTACAGAG |
| CaBAR1- | AACAGGGGCACAAACAGGCAAAAAACGGGCACAACCTCAATGGAGTGATGCAACC |
| tENO2, rev] | TGCCTGGAGTAAATGATGACACAAGGCAATTGACCCACGCATGTATCTATCTCAT |
| | TTTCTTACACCTTCTATTACCTTCTGCTCTCTCTGATTTGGAAAAAGCTGAAAAA |
| | AAAGGTTGAAACCAGTTCCCTGAAATTATTCCCCTACTTGACTAATAAGTATATA |
| | AAGACGGTAGGTATTGATTGTAATTCTGTAAATCTATTTCTTAAACTTCTTAAAT |
| | TCTACTTTTATAGTTAGTCTTTTTTTTAGTTTTAAAACACCAAGAACTTAGTTTC |
| | GAAGGATTCTAGAACTAGTAACatgcagttgagcaaggctgctgagatgtgttat |
| | gagataacaaactcttacttacacatagaccagaaatctcagataatagcaagta |
| | cacaagaagcgatacggttgacaagaaaatacttactaagtgaaatttttgtacg |
| | ttggagtccactgaatggggaaatatcattctcgtacaacggaggaaaagattgc |
| | caggtattactactgttatatctgagttgcttatgggaatatttcttcattaagg |
| | ctcaaaattcccaattcgatttcgagtttcaaagcttccccatgcaaagacttcc |
| | aactgttttcattgatcaagaagaaactttccctacattagagaattttgtactg |
| | gaaacctcagagcgatattgcctttccttatacgaatcacaaaggcaatctggtg |
| | catcggtcaatatggcagacgcatttagagattttataaagatataccctgaAac |
| | cgaagctatagtgataggtattagacacacagacccatttggtgaagcattaaag |
| | cctattcaaagaacagattctaactggcctgattttatgaggttgcaacctctct |
| | tacactgggacttaaccaatatatggagtttcttactgtattctaatgagccaat |
| | ttgtggactatatggtaaaggtttcacatcaatcggcggaattaacaactcattg |
| | cctaacccacacttgagaaaggactccaataatccagccttgcattttgaatggg |
| | aaatcattcatgcatttggcaaggacgcagaaggcgaacgtagttccgctataaa |
| | cacgtcacctatttccgtggtggataaggaaagattcagcaaataccatgacaat |
| | tactatcctggctggtatttggttgatgacactttagagagagcaggcaggatca |
| | agaattaaATTGAATTGAATTGAAATCGATAGATCAATTTTTTTCTTTTCTCTTT |
| | CCCCATCCTTTACGCTAAAATAATAGTTTATTTTATTTTTTGAATATTTTTTATT |
| | TATATACGTATATATAGACTATTATTTATCTTTTAATGATTATTAAGATTTTTAT |
| | TAAAAAAAAATTCGCTCCTCTTTTAATGCCTTTATGCAGTTTTTTTTTCCCATTC |
| | GATATTTCTATGTTCGGGTTCAGCGTATTTTAAGTTTAATAACTCGAAAATTCTG |
| | CGTTCGTTAAAGCTTTCGAGAAGGATATTATTTCGAAATAAACCGTGTTGTGTAA |
| | GCTTGAAGCCTTTTTGCGCTGCCAATATTCTTATCCATCTATTGTACTCTTTAGA |
| | TCCAGTATAGTGTATTCTTCCTGCCCCAAACGGGTAGTCCATCGTTGTAGGATAC |
| | TGTATATACACCCAGTAGAGTAGGTGACTACTGTATGAGCATACAGTAGTGACAA |
| | CCACTTACTGTATATAAATACAGTAGTGGTCATCGATACTGTATATAAAACCAGT |
| | AGGCAATCGTTTGTACTGTATGTACATACAGTATACCTCGCAACTACTGTATATA |
| | AACACAGTAATTAACTTGTAATATTCTAATCAATTGACAATATTATTTAAGGACC |
| | TATTGTTTTTTCCAATAGGTGGTTAGCAATCGTCTTACTTTCTAACTTTTCTTAC |
| | CTTTTACATTTCAGCAATATATATATATATATTTCAAGGATATACCATTCTAAGA |
| | TCTGGAAatgaagaaaaccgtagtgattggtgcaggttttggtggtttagctttg |
| | gctatacgtctacaagctgcaggtattcctacagtgctattggagcaaagagaca |
| | aaccaggaggaagagcttatgtttggcacgatcaaggctttacctttgatgctgg |
| | tcctacagtcatcactgatcctactgcattggaagctttgttcaccttagctggt |
| | agaagaatggaagattatgtccgtctattgcctgtcaagccgttttacagattgt |
| | gttgggaatctggtaaaaccctagattacgccaatgacagtgctgaactagaagc |
| | tcagattacgcagtttaatcccagagatgtcgaaggttacaggagattccttgcc |
| | tattcccaagctgttttccaagagggttatcttcgtttgggttcagttccattcc |
| | tgtcctttagggatatgcttagagcaggtcctcagttgttgaagctacaagcatg |
| | gcaaagtgtgtatcagtctgtttcgagatttatcgaggatgaacatctgagacaa |
| | gcattctcattccacagtcttctagttggaggtaatccctttaccacatcgagca |
| | tatatacgttgattcacgctttggaaagagaatggggagtttggtttcctgaagg |
| | tggaacaggtgctttggttaatggtatggtgaagctattcacggatttgggtgga |
| | gaaatagagctgaatgcaagagtggaagaacttgttgtagcagacaacagagtct |
| | cacaagttagacttgctgatggtaggatcttcgatacagatgctgtagcttcaaa |
| | cgcagatgtagtgaacacttataaaaagttgttgggacatcatcctgttggacaa |
| | aagagagcagctgctttggagaggaaatctatgagcaactcgttgtttgtccttt |
| | actttgggctgaatcaaccacactcacaactagctcatcacacaatctgctttgg |
| | tcctagatacagagagctgatagatgaaattttcactggatctgctttagcagac |
| | gatttttccctgtacttgcattcaccatgtgttactgatccctctttagcaccac |
| | ctggttgtgctagcttctatgtactagcacctgtaccacatttgggtaatgctcc |
| | attagattgggcacaagaaggaccgaaattgagggataggatcttcgactatttg |
| | gaagaacgttacatgccaggtttgagatctcagttggttacacagaggatattca |
| | caccagctgattttcatgatactctagatgcgcatttaggtagcgctttttccat |
| | tgagccacttttgacgcaaagtgcttggtttagaccacacaacagagattctgac |
| | attgccaatctgtacctagtaggtgcaggaactcatccaggagctggtattcctg |
| | gagttgtagcttctgctaaagctactgctagtctgatgatcgaggatttgcagta |
| | aTCTCTGCTTTTGTGCGCGTATGTTTATGTATGTACCTCTCTCTCTATTTCTATT |
| | TTTAAACCACCCTCTCAATAAAATAAAAATAATAAAGTATTTTTAAGGAAAAGAC |
| | GTGTTTAAGCACTGACTTTATCTACTTTTTGTACGTTTTCATTGATATAATGTGT |
| | TTTGTCTCTCCCTTTTCTACGAAAATTTCAAAAATTGACCAAAAAAAGGAATATA |
| | TATACGAAAAACTATTATATTTATATATCATAGTGT[ATTTTTCAAACTGCAAAT |
| | TCAAGAAAAAGCCACGCGTGTGCACCTTTTTTTTCCCCTTCCAGTGCATTATGCA |
| | ATAGACAGCACGAGTCTTTGAAAAAGTAACTTATAAAACTGTATCAATTTTTAAA |
| | CCTAAATAGATTCATAAACTATTCGTTAATATAAAGTGTTCTAAACTATGATGAA |
| | AAAATAAGCAGAAAAGACTAATAATTCTTAGTTAAAAGCACTCTCGAGTTAACTA |
| | GTttaacaatactgagggttgcttgtgacggaagtaatcttatcatcacttgtgc |
| | tgccgctcgttaggttgctagaggtaccctcactgatagaaaaggatgaataggt |
| | gctttggacctctgtcggtgcaacggttaccgtggtttctgattccgcgatatat |
| | ataacgctataagtgtcctcaacatcgggcacgtctactgtcggtactggtagct |
| | ggtaaccattctccacagcgacgacgttcgcctctccgttagaattcttatattt |
| | accgaaatatatttgtgcgttgtcgtagtcaaatatagcatagtggtacttaaaa |
| | atcaaatttggcagcagaccttcataggcgttatagcctatatcgcatgaagcta |
| | catttaaggctacgaatgtagttgagttcctttcaatggtatcgccgactatgtc |
| | gaccagctggaagccaatgtcgtacccttgtactgaaaatgttatatttaacccc |
| | tctatgtacttgatgtcgaaatatatattgccattgtcatcctggatgtagtcgt |
| | cgcctaaaattttaaggatgttattaaggaccggagttggggggacgaaaccgtt |
| | agtgccactatcgatttggtaaagcgtcttctggttgctaacgatggtgtctccc |
| | aacttgatctgatttactgtgaacgttggcatcaaccagtagtcaagctggttta |
| | tgaaggatatcactttgataaagggcgctcttactaaaggcccgtctatgatgtt |
| | cgtaattatccctccgaagataatggacgggttctgatcctgaccattgaatgag |
| | aatagaacacttttcgtataaccctggtctttaagtagataggggaagttactgt |
| | aattcgttttaaaaagttcttgacgagtaaatccaatgcctagtataccggaaaa |
| | actgtccatatcagaaatattggccagtcccatagatagattagacagtttccag |
| | tcgttactcctaatagtttcattgatgaagtataccgggaactgtcccccgtatc |
| | catatttcataattgcggtctggttggagttatagaaacttgatgacgcagagaa |
| | gttgtatatggagtcaactatcataagatcgctacttcctgaatcgactataacg |
| | ttagatatgaactggggcggcgtccctaacgctaaattctctatgtaatacatgt |
| | gctcatcggatatcgtaaggtctaatttcaccgcaccgccggtattggatacggc |
| | cacggctagggatgggacaagaagtgaaagaatagtaaataacatACTAGTGATC |
| | TTTATTGATATAGTGTTTAAGCGAATGACAGAAGATTAATTTCTTGGTATGTTAG |
| | GAAAGAATAAAGGAGAATAAGAATAATTAGAACAATGTAGGATGGAAAGAAAGAT |
| | TATCAAGCATGCCGACTTTATATACTTGAACGGAGGCAAAGGATGCAAAATTTTC |
| | TCACATTTCTTTCTGCCGTTATGTTGGAAGTAAGACTCCCATTATCGCAATACTG |
| | CAACACGAATATGCAAAATTTGCTGAGTTATCGCAGATAGTTGTTGCAAAGATAG |
| | CGGCGTAGGTGGCCGCGAAATGGGAAATTCCAAAACAAACGGTTTTTTTACTCCT |
| | GAGAAATACTTGTACGGGATAATCCAGGGCCTACCACCCACGCTTCGAGGATTGG |
| | CTTTTATTTTTTTTTTTTTGGTGGCGTTTTATTTCTTTCCCGCTTTCTGGGACTT |
| | GTGCGGAGTTTTGAGAGGGGCGCGCGGCAAAGGATTCCCAAAACGGAAATCAGAC |
| | GCCAATAGCCAGCACTCAAAGCAGTTCTGGACCCATTCCGATTTTCCCATTTGGT |
| | TCTTGCGCGTGCTGATTCCGACACGCGCGTCTATAAATAGCATGAAGTATCCGCA |
| | CACCGCAGCGTTAGTGAGGTGAGGGTGGCAGCAAGCTAATTCCCGCATCTGGAAT |
| | CTGAACTGCCCCTTTTGGACTAACCGTGTGGTTCATGGGTG]TTTTCGAGACC |

Readout ORFs

| | |
|---|---|
| ym Turquoise2 | Atggttagtaaaggtgaagaattgttcactggtgtagtgccaattttagtagaat |
| | tagatggtgacgttaacggtcataagttctctgtaagcggtgaaggtgaaggtga |
| | cgcaacatacggtaaattaactttgaaattcatttgtactacaggtaaattacca |
| | gttccatggccaactttggttactactttgtcttggggtgttcaatgtttcgcta |
| | gatacccagatcatatgaagcaacatgatttctttaagtctgctatgccagaagg |
| | ttacgttcaagaaagaactatcttcttcaaggatgatggtaactataagactaga |
| | gctgaagttaagttcgaaggagatactttagttaatagaattgaattgaagggta |
| | ttgattttaaggaagatggtaatattttgggtcataagttggaatataattactt |
| | ctctgataacgtttacattactgctgataagcaaaagaacggtattaaggcgaac |
| | ttcaagattagacataacattgaagatggtggtgttcaattagctgatcattacc |
| | aacaaaacactccaattggtgacggtccagttttgttgccagataaccattactt |
| | gtctactcaatctaagttgtctaaggatccaaacgaaaagagagatcatatggtt |
| | ttgttggaattcgttactgctgctgggatcactttaggtatggatgaattgtata |
| | aatag |
| CrtI | atgaagaaaaccgtagtgattggtgcaggttttggtggtttagctttggctatac |
| | gtctacaagctgcaggtattcctacagtgctattggagcaaagagacaaaccagg |
| | aggaagagcttatgtttggcacgatcaaggctttacctttgatgctggtcctaca |
| | gtcatcactgatcctactgcattggaagctttgttcaccttagctggtagaagaa |
| | tggaagattatgtccgtctattgcctgtcaagccgttttacagattgtgttggga |
| | atctggtaaaaccctagattacgccaatgacagtgctgaactagaagctcagatt |
| | acgcagtttaatcccagagatgtcgaaggttacaggagattccttgcctattccc |
| | aagctgttttccaagagggttatcttcgtttgggttcagttccattcctgtcctt |
| | tagggatatgcttagagcaggtcctcagttgttgaagctacaagcatggcaaagt |
| | gtgtatcagtctgtttcgagatttatcgaggatgaacatctgagacaagcattct |
| | cattccacagtcttctagttggaggtaatccctttaccacatcgagcatatatac |
| | gttgattcacgctttggaaagagaatggggagtttggtttcctgaaggtggaaca |
| | ggtgctttggttaatggtatggtgaagctattcacggatttgggtggagaaatag |
| | agctgaatgcaagagtggaagaacttgttgtagcagacaacagagtctcacaagt |
| | tagacttgctgatggtaggatcttcgatacagatgctgtagcttcaaacgcagat |
| | gtagtgaacacttataaaaagttgttgggacatcatcctgttggacaaaagagag |
| | cagctgctttggagaggaaatctatgagcaactcgttgtttgtcctttactttgg |
| | gctgaatcaaccacactcacaactagctcatcacacaatctgctttggtcctaga |
| | tacagagagctgatagatgaaattttcactggatctgctttagcagacgattttt |
| | ccctgtacttgcattcaccatgtgttactgatccctctttagcaccacctggttg |
| | tgctagcttctatgtactagcacctgtaccacatttgggtaatgctccattagat |
| | tgggcacaagaaggaccgaaattgagggataggatcttcgactatttggaagaac |
| | gttacatgccaggtttgagatctcagttggttacacagaggatattcacaccagc |
| | tgattttcatgatactctagatgcgcatttaggtagcgctttttccattgagcca |
| | cttttgacgcaaagtgcttggtttagaccacacaacagagattctgacattgcca |
| | atctgtacctagtaggtgcaggaactcatccaggagctggtattcctggagttgt |
| | agcttctgctaaagctactgctagtctgatgatcgaggatttgcagtaa |

List of expression modules and part sequences constructed in this example. Promoters and terminators are in upper case, open reading frames (ORFs) in lower case. BsaI restriction enzyme recognition site is highlighted grey. Golden Gate Assembly 4-bp overlaps are in bold.

Golden Gate Assembly of communication cassettes into yeast genomic Integration plasmid: A Golden Gate reaction mixture was prepared as follows: appropriate volume of each DNA plasmid in 3:1 ratio with integration plasmid set to 10 ng, 0.75 μL T4 DNA Ligase buffer, 0.75 μL T4 DNA Ligase, 0.45 μL BsaI restriction enzyme, 0.075 μL BSA and water to bring the final volume to 7.5 μL. Reaction mixtures were incubated in a thermocycler according to the following program: 60 cycles of digestion and ligation (37° C. for 3 min, 16° C. for 4 min) followed by a final digestion step (37° C. for 60 min), and a heat inactivation step (80° C. for 5 min). The mixture is then diluted 1:3 with water and E. coli is transformed via electroporation and plated on LB/Amp plates. Correctly assembled plasmids are screened and verified using colony PCR and Sanger sequencing. Correctly transformed colony was grown in LB/Amp overnight and stocked with 20% glycerol at −80° C.

CRISPR/Cas9 system and genomic integration: CRISPR/Cas9 gRNA sequences are available in Table 15. In a typical genomic integration of assembled integration plasmids using CRISPR/Cas9, the plasmids were digested with NotI restriction enzyme and directly used along with Cas9/gRNA plasmid (URA3 or NAT selection marker) following lithium acetate yeast transformation method. Transformed colonies are grown on selective media for the selection marker integrated along with the remaining biosensor components and for the Cas9/gRNA plasmid. Correctly integrated colonies are screened phenotypically when possible, by activating integrated components with appropriate synthetic peptide and following appropriate fluorescence/color development, and sequence is verified using PCR amplification and Sanger sequencing from purified genomic DNA of the screened colony. Transformed colony was cured from Cas9/gRNA plasmid by growing in non-selective media and replica plating. Single cured colony was grown in YPD overnight and stocked with 15% glycerol at −80° C.

TABLE 15

| Target gene or locus | Sequence 5′→3′ |
|---|---|
| STE2 gene in y WS890 | TCACCTCTACTGATGAACTG |
| URA3 gene in y WS890 | GAAGTAACAAAGGAACCTAG |
| LEU2 gene in y WS890 | TTTTTGAACACACATGAACA |
| ARS208a locus | GTCCGCTAAACAAAAGATCT |
| HO locus | AGAATTGATTGCTGCTTATG |
| LEU2 locus | TAACATTAATATTGACAAGG |

Biosensor activation assay and dose-response assay using fluorescent readout: Glycerol stocked strains were streaked out on YPD plate and incubated for two days. Three colonies were picked, replated on YPD plate, incubated for one day and then stored at 4° C. For activation assays, a small scoop of yeast patch from the three colonies was resuspended in ˜4 mL YPD media and incubated overnight. Overnight cells were seeded at an OD₆₀₀ of 0.15 (as measured in 96-well plate at 200-μL volume) in SC/Glc media after washing twice with sterile MiliQ H₂O. GPCR activity and response to increasing concentrations of synthetic peptide ligands was measured in strains carrying appropriate genome integrated components in 96-well microtiter plates in 200 μL total volume, cultured at 30° C. and 800 rpm, unless otherwise stated. All measurements were performed in biological triplicate. Culture turbidity (OD₆₀₀) and fluorescence for ymTurquoise2 (excitation: 439 nm, emission: 475 nm, gain: 75) were measured after 0 and 8 h using an Infinite 200 Pro plate reader (Tecan). To account for the optical density values exceeding the linear range of the photo detector, all optical density values were corrected with the following formula to yield true optical density values:

$${A}_{true} = \frac{k * {A}_{meas}}{{A}_{sat} - {A}_{meas}}$$

Where Aₛₐₜ is the photodetector saturation value, Aₘₑₐₛ is the measure optical density, and k is the true optimal density at which the detector reaches half saturation of the measured optical density [18]. Dose-response was measured at different concentrations (eleven x-fold dilutions in H₂O starting with maximal peptide concentration and H₂O was used as no peptide control) of the appropriate synthetic peptide ligand. All fluorescence values were normalized by the A₆₀₀, and plotted against the log (10)-converted peptide concentrations. Data were fit to a four-parameter non-linear regression model using Prism (GraphPad) in order to extract GPCR-specific values for basal activation, maximal activation, EC₅₀, and the Hill coefficient.

Biosensor activation assay and dose-response assay using lycopene readout strains: Induction of lycopene was assayed in transparent 96-well microtiter plates cultured at 30° C. and 800 rpm. Cells were seeded at an OD₆₀₀ of 2 as measured by cuvette in SC/Glc media. All measurements were performed in biological triplicate. Relative lycopene content was calculated by spectroscopy as described previously [18]. Optical densities were measured with an Infinite M200 plate reader (Tecan). Lycopene values were normalized by the culture OD₆₀₀ to give a measure of lycopene per cell.

Quantification and statistical analysis: Statistical tests of all experiments were performed using GraphPad Prism version 7 are detailed within the legend of each figure. In all figures, the data points represent mean±SD of biological triplicates. Curves fitted to all dose-response data was fitted in Prism 7 using the Nonlinear Regression: Variable slope (four parameter) curve fitting.

### Results

Fungal peptide proteases are selective for their cognate fungal pheromones. In order to identify proteases that could distinguish wild-type and variant epitopes, a toolbox of functional fungal proteases that cleave their native mating peptides was needed. First, characterized fungal pheromone proteases were identified from the literature. Saccharomyces cerevisiae's Bar1 aspartyl protease (ScBar1) [19], Candida albicans' Bar1 aspartyl protease (CaBar1) [20] and Schizosaccharomyces pombe's Sxa2 serine carboxypeptidase (SpSxa2) [21] had all been previously reported, so first these GPCRs and synthesized their corresponding peptides were cloned. Through inspection of sequence homology to these proteases, two additional putative pheromone proteases were further identified: Schizosaccharomyces japonicus' serine carboxypeptidase Sxa2 (SjSxa2; 43% identity to SpSxa2) and Schizosaccharomyces octosporus' serine carboxypeptidase Sxa2 (SoSxa2; 60% identity to SpSxa2). The complementary pheromone GPCRs (ScSte2, CaSte2, SpSte2, SjSte2 and SoSte2, respectively) were all cloned and reported previously and the orthogonal pheromone peptides (ScPep, CaPep, SpPep, SjPep and SoPep, respectively) were all reported and made by standard solid-phase peptide synthesis [14]. The amino acid sequences of these five proteases, GPCRs, and peptides are all given in Tables 16, 17 and 19, respectively.

TABLE 16

| Species | |
|---|---|
| (protease, accession | |
| number) | Amino acid sequence |
| Saccharomyces | MSAINHLCLKLILASFAIINTITALTNDGTGHLEFLLQHEEEMYYAT |
| cerevisiae [35] | T [symbol] TPSQSLTVLFDTGSADFWVMDSSNPFCLPNSNTSSYSNATYNGEE |
| (ScBar1, NP_012249) | VKP [symbol] RSMSTYNEHRSSTYQYLENGRFYITYADGTFADGSWGTETVSI |
| | NGIDI [symbol] FGVAKYATTPVSGVLGIGFPRRESVKGYEGAPNEYYPNFPQ |
| | ILKSEKI [symbol] AYSLFLNSPDSGTGSIVFGAIDESKFSGDLFTFPMVNEY |
| | PTIVDAPAT [symbol] IQGLGAQNKSSCEHETFTTTKYPVLLDSGTSLLNAPK |
| | VIADKMASFVN [symbol] EEEGIYILDCPVSVGDVEYNFDFGDLQISVPLSSL |
| | ILSPETEGSYCGF [symbol] TNDSMVLGDVFLSSAYVVFDLDNYKISLAQANW |
| | NASEVSKKLVNIQTD [symbol] GAKIATAEPWSTNEPFTVTSDIYSSTGCKSR |
| | PFLQSSTASSLIAETNV [symbol] CSTKMPGTRSTTVLSKPTQNSAMHQSTGA |
| | VTQTSNETKLELSSTMANS [symbol] LPTSNSIDKEFEHSKSQTTSDPSVAEH |
| | STFNQTFVHETKYRPTHKTVI [symbol] TKYSTVLINVCKPTY |
| Candida albicans | MLFTILSLLVPSLAVAVSNTGGAVKLDLTISDEHMYYIENLALGTPP |
| [26] | QFISNVIVDSGSSDLMIVDSIYNFSASSSFYNSNQTAIMKYGYGGQF |
| (CaBar1, KGQ96729) | PVYFINETIRSNDWKLSNLSMGLANISDMDSFSGILGIGFTRQELFK |
| | TNYSNFPYLLKDQGYTKSVLFSENGQDQNPSIIFGGIITNIIDGPLV |
| | RAPFIKVISFINQLDYWLMPTFTVNQIKLGDTIVSNQKTLYQIDSGT |
| | NGFVPPTPVLNNILKILGDDYIQDDNGNIYFDIKYIEGLNITFSVQG |
| | YDIGFQLVDIVGDTIERNSTTFVALNVASCDIGYNAYEGLLPNLIFK |
| | YHYAIFDYDNAQIYFGKYKNSNGEANVVAVENGYQLPVPTVDVPDVE |
| | DTYSVIYIAESETTVTVAPTEVQSTYSSFSISEGTSSNLTSGSTSDD |
| | KITSVTSNPQYC |
| Schizosaccharomyces | MLSLFLKSLFAIIIIELTIIHALPTYTVHWKCSIQQANTSSASSNQT |
| pombe [36] | VQPRQHAAPSSDRIKSLPEFKGSLPELYSGYLEANSDKSLFYTYAPA |
| (SpSxa2, NP_593043) | VVDSETFIVWLQGGPGCAGTLGFFSENGPIEISQSSPSPSLNPESWT |
| | NFANMLWLDQPFGTGYSQGQAAYTTTIEEASSDFVNALKSFYQKEPH |
| | LMKKKLYLVGESYGSIWSANFAEALLSEPSLNINFMGVGIVSGLTAD |
| | YETQEQITASIWVEHISKLGYYFNNTSSTISEEFKKRNKECQYDSVL |
| | NRLTFPTEQYPIWRPEYNFSTSTSLRKREALDGEDIGNVENSISGCD |
| | LYSLSNFLLYLENSCVITYDVSLDCSFNEYNDPLITYLNREDVRSSL |
| | HATKASTALTSGEGVFADGCNFDLYKKIVSNNVESVLVEIIPRLTEK |
| | YKVSFLAGALDLQILWTGTLLALQNTTWNGWQGFTQSPGSLETTNGF |
| | TLDERNLAFTLSNSVGHMAPSKDPQMVREWLENTLLY |
| Schizosaccharomyces | MNLAVHVLLLSFLLYTVHSFPAYPPYPIYPRSSLNSSNASSVPIPQP |
| octosporus | SYNSSSKAMNPDLVTSLPEYSGSLPELYSGYLSAENFKQIFYTFAPA |
| (SoSxa2, | ITPTDKFIVWLQGGPGCAGTLGFFSENGPIILDPNHQTPQDNPNSWT |
| XP_013015964) | RFANMMWIDQPFGTGYSQGSPLASTVSIATSDFIQTLKSFYAKFPEM |
| | KEKELYLVGESYGSVWGAYFAQALRSDPELHAIPFGGLGIISGLLSD |
| | YETQNTITASMWLDHVNKMGLYYTNESAEVSTFFAAKNQQCGYAEVL |
| | ERMQFPAEQTPIPKPRVNLTNSFSKKKRQADSDSDSGNIKEAFQMIT |
| | DCDTYTLTDFLLYLKNHCMITYDITLDCSENSNNDPLTTYLNRKDVQ |
| | KQLHAIHVESALTSSKGIFGNGCNYDVYGDIVKNGAPSILEEAIPEL |
| | VQNYKVSFLAGALDFQLLWTGSLLALQNTTWNGWQGFQEIPDLNSPF |
| | GFTLDERNLAFTLSNKVGHMAPSKDPSMVIDWLQKTLLG |
| Schizosaccharomyces | MVVLHRLLLSLWPAGAMLLVSSELLNLPTTSAIPIYSDPHQCSLRVS |
| japonicus | VRTDVEGEQQQTQAMGSVVIASNVTSSDAPTENPTSSAPTTFSHTVN |
| (SjSxa2, | STFPTHTPTTSTSTNTPTDARVSSLPLLNGDMPALYAGYLNVSASST |
| XP_002172242) | SPHSIFYAYAPSEQGSDDLIVWLQGGPGCAGTIGLFAENGPVRFGPG |
| | DSAPVRSEHSWTRYADVLYLDQPFGTGFSYAVTEADYTTTMDAASAE |
| | FIAFLDGFFDAYPDTASKRLFFVGESYGSFWGAYFARALADRPDIYD |
| | RIVSVGIVSGLSTTHDTQYSVSNSLWLSHLNKLGYYTQDKSTWNSES |
| | RVNARCGYDKVMQEFTFPASQTPLSSISTSTSSSSTSSGAGAGATVS |
| | SLTDSSGNASYTNSSSSAEVDVFSYFPSCDTNDATYMLMYLQNTCLI |
| | SYDVSIDCTTASQLTDHLTPYLNRADVREALHVPSSVAAFSTANGVF |
| | GDGCNYDLLQKINTVTYEPVLKSVVPSLIADGKRVAFLSGALDYQIP |
| | WSGTLLAMQNTTWAGWQGFQTEPDWDNPVGFTLNERGLSFTLLNSVG |
| | HMASMNDPDRVNRWLQEQVL |

[symbol]

indicates data missing or illegible when filed

TABLE 17

| Species | |
|---|---|
| (GPCR, accession number) | Amino acid sequence |
| Saccharomyces cerevisiae | MSDAAPSLSNLFYDPTYNPGQSTINYTSIYGNGSTITFDELQ |
| [37] (ScSte2, D6VTK4) | GLVNSTVTQAIMFGVRCGAAALTLIVMWMTSRSRKTPIFIIN |
| | QVSLFLIILHSALYFKYLLSNYSSVTYALTGFPQFISRGDVH |
| | VYGATNIIQVLLVASIETSLVFQIKVIFTGDNFKRIGLMLTS |
| | ISFTLGIATVTMYFVSAVKGMIVTYNDVSATQDKYFNASTIL |
| | LASSINFMSFVLVVKLILAIRSRRELGLKQFDSFHILLIMSC |
| | QSLLVPSIIFILAYSLKPNQGTDVLTTVATLLAVLSLPLSSM |
| | WATAANNASKTNTITSDFTTSTDRFYPGTLSSFQTDSINNDA |
| | KSSLRSRLYDLYPRRKETTSDKHSERTFVSETADDIEKNQFY |
| | QLPTPTSSKNTRIGPFADASYKEGEVEPVDMYTPDTAADEEA |
| | RKFWTEDNNNL |
| Candida albicans [14] | MNINSTFIPDKPGDIIISYSIPGLDQPIQIPFHSLDSFQTDQ |
| (CaSte2) | AKIALVMGITIGSCSMTLIFLISIMYKTNKLTNLKLKLKLKY |
| | ILQWINQKIFTKKRNDNKQQQQQQQQQIESSSYNNTTTTLGG |
| | YKLFLFYLNSLILLIGIIRSGCYLNYNLGPLNSLSFVFTGWY |
| | DGSSFISSDVTNGFKCILYALVEISLGFQVYVMFKTSNLKIW |
| | GIMASLLSIGLGLIVVAFQINLTILSHIRFSRAISTNRSEEE |
| | SSSSLSSDSVGYVINSIWMDLPTILFSISINIMTILLIGKLI |
| | IAIRTRRYLGLKQFDSFHILLIGFSQTLIIPSIILVVHYFYL |
| | SQNKDSLLQQISLLLIILMLPLSSLWAQTANNTHNINSSPSL |
| | SFISRHHLSDSSRSGGSNTIVSNGGSNGGGGGGGNFPVSGID |
| | AQLPPDIEKILHEDNNYKLLNSNNESVNDGDIIINDEGMITK |
| | QITIKRV |
| Schizosaccharomyces | MRQPWWKDFTIPDASAIIHQNITIVSIVGEIEVPVSTIDAYE |
| pombe [14] | RDRLLTGMTLSAQLALGVLTILMVCLLSSSEKRKHPVFVENS |
| (SpSte2, NP_594722) | ASIVAMCLRAILNIVTICSNSYSILVNYGFILNMVHMYVHVF |
| | NILILLLAPVIIFTAEMSMMIQVRIICAHDRKTQRIMTVISA |
| | CLTVLVLAFWITNMCQQIQYLLWLTPLSSKTIVGYSWPYFIA |
| | KILFAFSIIFHSGVFSYKLFRAILIRKKIGQFPFGPMQCILV |
| | ISCQCLIVPATFTIIDSFIHTYDGFSSMTQCLLIISLPLSSL |
| | WASSTALKLQSMKTSSAQGETTEVSIRVDRTFDIKHTPSDDY |
| | SISDESETKKWT |
| Schizosaccharomyces | MREPWWKNYYTMNGTQVQNQSIPILSTQGYIQVPLSTIDKAE |
| octosporus [14] | RNRILTGMTVSAQLALGVLIMVMSILLSSPEKRKTPVFIVNS |
| (SoSte2, XP_013018801) | ASIISMCIRAILMIVNLCSESYSLAVMYGFVFELVGQYVHVF |
| | DILVMIIGTIIIITAEVSMLLQVRIICAHDRKTQRIVTCISS |
| | GLSLIVVAFWFTDMCQEIKYLLWLTPYNNHQISGYYWVYFVG |
| | KILFAVSIMFHSAVFSYKLFHAIQIRKKIGQFPFGPMQCILI |
| | ISCQCLFVPAIFTIIDSFIHTYDGFSSMTQCLLIVSLPLSSL |
| | WASSTALKLQSLKSTTSPGDTTQVSIRVDRTYDIKRIPTEEL |
| | SSVDETEIKKWP |
| Schizosaccharomyces | MYSWDEFRSPKQAEVLNQTVTLETIVSTIQLPISEIDSMERN |
| japonicus [14] | RLLTGMTVAVQVGLGSFILVLMCIFSSSEKRKKPVFIFNFAG |
| (SjSte2, XP_002173203) | NLVMTLRAIFEVIVLASNNYSIAVQYGFAFAAVRQYVHAFNI |
| | IILLLGPFILFIAEMSLMLQVRIICSQHRPTMITTTVISCIF |
| | TVVTLAFWITDMSQEIAYQLFLKNYNMKQIVGYSWLYFIAKI |
| | TFAASIIFHSSVFSFKLMRAIYIRRKIGQFPFGPMQCIFIVS |
| | CQCLIVPAIFTLIDSFTHTYDGFSSMTQCLLIISLPLSSLWA |
| | THTAQKLQTMKDNTNPPSGTQLTIRVDRTFDMKFVSDSSDGS |
| | FTEKTEETLP |

The next step was to engineer yeast biosensor strains having all of the necessary components to test the functionality of the proteases—the protease, the GPCR, and the fluorescent reporter. The yWS890 strain engineered by Shaw et al. which has remodeled and optimized pheromone signaling pathway was genomically modifed [22]. This strain has native S. cerevisiae pheromone GPCR and pheromone-inducible expression of super-folder Green Fluorescent Protein (sfGFP). In order to make a parent strain where GPCR, protease and a readout of interest can be modularly combined, Ste2, sfGFP, LEU2 and URA3 along with their corresponding promoters and terminators was deleted in the yWS890 genome. LEU2 and URA3 selection markers were deleted, since their auxotrophy is needed for the CRISPR/Cas9 genomic integration selections (see Methods). The engineered parent yeast strain (yTC370) has a remodeled and optimized pheromone signaling pathway and is lacking only the three expression components (GPCR, inducible readout and protease) in order to complete the functional peptide-inducible GPCR response system. Next, yeast strains from the parent strain were engineered to express all relevant biosensing components (GPCR, fluorescent or lycopene readout and protease).

In order to simply mix-and-match desired combinations of the protease and the GPCR, codon-optimized open reading frames (ORFs) of all five proteases and GPCRs along with appropriate promoters and terminators were cloned into standard Golden Gate Assembly (GGA, see Materials and Methods) cassette plasmids using Gibson assembly (Tables 14 and 18). Strong constitutive promoters for GPCRs and proteases were selected and orthogonally inducible LexO(6×)-pLEU2m promoter for ymTurquoise2 fluorescent protein readout expression. For all ORFs, standard characterized yeast terminators were selected [23]. The cassette plasmids are a modification of the MoClo Yeast Toolkit plasmids [23] where each cassette plasmid contains a full transcriptional unit which can be modularly assembled into a multigene plasmid using GGA. The cassette plasmids were constructed in order to assemble a complete activation pathway (constitutive GPCR expression, inducible readout expression and constitutive protease expression) into a genomic integration plasmid that can be transformed into parent yeast strain yTC370. Sequences of promoters, terminators, ORFs and GGA overlaps are available in Table 14.

TABLE 18

| Sequence | |
|---|---|
| name | Nucleotide sequence |
| ScSte2 | ATGTCTGATGCGGCTCCTTCATTGAGCAATCTATTTTATGATCCAACGTATAATCCTGG |
| | TCAAAGCACCATTAACTACACTTCCATATATGGGAATGGATCTACCATCACTTTCGATG |
| | AGTTGCAAGGTTTAGTTAACAGTACTGTTACTCAGGCCATTATGTTTGGTGTCAGATGT |
| | GGTGCAGCTGCTTTGACTTTGATTGTCATGTGGATGACATCGAGAAGCAGAAAAACGCC |
| | GATTTTCATTATCAACCAAGTTTCATTGTTTTTAATCATTTTGCATTCTGCACTCTATT |
| | TTAAATATTTACTGTCTAATTACTCTTCAGTGACTTACGCTCTCACCGGATTTCCTCAG |
| | TTCATCAGTAGAGGTGACGTTCATGTTTATGGTGCTACAAATATAATTCAAGTCCTTCT |
| | TGTGGCTTCTATTGAGACTTCACTGGTGTTTCAGATAAAAGTTATTTTCACAGGCGACA |
| | ACTTCAAAAGGATAGGTTTGATGCTGACGTCGATATCTTTCACTTTAGGGATTGCTACA |
| | GTTACCATGTATTTTGTAAGCGCTGTTAAAGGTATGATTGTGACTTATAATGATGTTAG |
| | TGCCACCCAAGATAAATACTTCAATGCATCCACAATTTTACTTGCATCCTCAATAAACT |
| | TTATGTCATTTGTCCTGGTAGTTAAATTGATTTTAGCTATTAGATCAAGAAGATTCCTT |
| | GGTCTCAAGCAGTTCGATAGTTTCCATATTTTACTCATAATGTCATGTCAATCTTTGTT |
| | GGTTCCATCGATAATATTCATCCTCGCATACAGTTTGAAACCAAACCAGGGAACAGATG |
| | TCTTGACTACTGTTGCAACATTACTTGCTGTATTGTCTTTACCATTATCATCAATGTGG |
| | GCCACGGCTGCTAATAATGCATCCAAAACAAACACAATTACTTCAGACTTTACAACATC |
| | CACAGATAGGTTTTATCCAGGCACGCTGTCTAGCTTTCAAACTGATAGTATCAACAACG |
| | ATGCTAAAAGCAGTCTCAGAAGTAGATTATATGACCTATATCCTAGAAGGAAGGAAACA |
| | ACATCGGATAAACATTCGGAAAGAACTTTTGTTTCTGAGACTGCAGATGATATAGAGAA |
| | AAATCAGTTTTATCAGTTGCCCACACCTACGAGTTCAAAAAATACTAGGATAGGACCGT |
| | TTGCTGATGCAAGTTACAAAGAGGGAGAAGTTGAACCCGTCGACATGTACACTCCCGAT |
| | ACGGCAGCTGATGAGGAAGCCAGAAAGTTCTGGACTGAAGATAATAATAATTTATAG |
| CaSte2 | ATGAATATCAATTCAACTTTCATACCTGATAAACCAGGCGATATAATTATTAGTTATTC |
| | AATTCCAGGATTAGATCAACCAATTCAAATTCCTTTCCATTCATTAGATTCATTTCAAA |
| | CCGATCAAGCTAAAATAGCTTTAGTCATGGGGATAACTATTGGGAGTTGTTCAATGACA |
| | TTAATTTTTTTGATTTCTATAATGTATAAAACTAATAAATTAACAAATTTAAAATTAAA |
| | ATTAAAATTAAAATATATCTTGCAATGGATAAATCAAAAAATCTTCACCAAAAAAAGGA |
| | ATGACAACAAACAACAACAACAACAACAACAACAACAAATTGAATCATCATCATATAAC |
| | AATACTACTACTACGCTGGGGGGTTATAAATTATTTTTATTTTATCTTAATTCATTGAT |
| | TTTATTAATTGGTATTATTCGATCAGGTTGTTATTTAAATTATAATTTAGGTCCATTAA |
| | ATTCACTTAGTTTTGTATTTACTGGTTGGTATGATGGATCATCATTTATATCATCCGAT |
| | GTAACTAATGGATTTAAATGTATTTTATATGCTTTAGTGGAAATTTCATTAGGTTTCCA |
| | AGTTTATGTGATGTTCAAAACTTCAAATTTAAAAATTTGGGGGATAATGGCATCATTAT |
| | TATCAATTGGTTTAGGATTGATTGTTGTTGCCTTTCAAATCAATTTAACAATTTTATCT |
| | CATATTCGATTTTCCCGGGCTATATCAACTAACAGAAGTGAAGAAGAATCATCATCATC |
| | ATTATCATCTGATTCGGTTGGGTATGTGATTAATTCAATATGGATGGATTTACCAACAA |
| | TATTATTTTCCATTAGTATTAATATAATGACAATATTATTGATTGGTAAACTTATAATT |
| | GCTATTAGAACAAGACGTTATTTAGGATTGAAACAATTTGATAGTTTCCATATTTTATT |
| | AATTGGTTTCAGTCAAACATTAATTATTCCTTCAATTATTTTGGTGGTTCATTATTTTT |
| | ATTTATCACAAAATAAAGATTCTTTATTACAACAAATTAGTCTTTTATTGATTATTTTA |
| | ATGTTACCATTAAGTTCTTTATGGGCTCAAACTGCTAATAATACTCATAATATTAATTC |
| | ATCTCCAAGTTTATCATTCATATCTCGTCATCATCTGTCTGATAGTAGTCGTAGTGGTG |
| | GTTCCAATACAATTGTTAGTAATGGTGGTAGTAATGGTGGTGGTGGTGGTGGTGGGAAT |
| | TTCCCTGTTTCAGGTATTGATGCACAATTACCACCTGATATTGAAAAAATCTTACATGA |
| | AGATAATAATTATAAATTACTTAATAGTAATAATGAAAGTGTAAATGATGGAGATATTA |
| | TCATTAATGATGAAGGTATGATTACTAAACAAATCACCATCAAAAGAGTGTAG |
| SpSte2 | ATGAGACAACCATGGTGGAAAGACTTTACTATTCCCGATGCATCCGCAATTATTCACCA |
| | AAATATTACCATTGTCTCTATTGTAGGAGAGATTGAAGTGCCAGTTTCAACAATTGATG |
| | CATATGAAAGAGATAGACTTTTAACTGGAATGACTTTGTCTGCCCAACTTGCTTTAGGA |
| | GTCCTTACCATTTTGATGGTTTGTCTATTGTCATCATCCGAAAAACGAAAACACCCAGT |
| | TTTTGTTTTTAATTCGGCAAGTATTGTTGCAATGTGTCTTCGGGCCATTTTGAATATAG |
| | TGACCATATGCAGCAATAGCTACAGTATCCTGGTTAATTACGGGTTTATCTTAAACATG |
| | GTTCATATGTATGTCCATGTGTTTAATATTTTAATTTTGTTGCTTGCACCGGTCATCAT |
| | TTTTACTGCTGAGATGAGCATGATGATTCAAGTTCGTATAATTTGTGCACATGATAGAA |
| | AGACACAAAGGATAATGACTGTTATTAGTGCCTGCTTAACTGTTTTGGTTCTCGCATTT |
| | TGGATTACTAACATGTGTCAACAGATTCAGTATCTGTTATGGTTAACTCCACTTAGCAG |
| | CAAGACCATTGTTGGATACTCTTGGCCCTACTTTATTGCTAAAATACTTTTTGCTTTTA |
| | GCATTATTTTTCACAGTGGTGTTTTTTCATACAAACTCTTTCGTGCCATATTAATACGG |
| | AAAAAAATTGGGCAATTTCCATTTGGTCCGATGCAGTGTATTTTAGTTATTAGCTGCCA |
| | ATGTCTTATTGTTCCAGCTACCTTTACTATAATAGATAGTTTTATCCATACGTATGATG |
| | GCTTTAGCTCTATGACTCAATGTCTGCTAATCATTTCTCTTCCTCTTTCGAGTTTATGG |
| | GCGTCTAGTACAGCTCTGAAATTGCAAAGCATGAAAACTTCATCTGCGCAAGGAGAAAC |
| | CACCGAGGTTTCGATTAGAGTTGATAGAACGTTTGATATCAAACATACTCCCAGTGACG |
| | ATTATTCGATTTCTGATGAATCTGAAACTAAAAAGTGGACGTAG |
| SoSte2 | ATGCGTGAACCATGGTGGAAGAACTACTACACCATGAACGGTACCCAAGTCCAAAACCA |
| | ATCCATCCCAATTTTGTCCACCCAAGGTTACATTCAAGTTCCATTGTCCACCATCGATA |
| | AGGCTGAAAGAAACAGAATTTTGACTGGTATGACCGTTTCTGCTCAATTGGCCTTGGGT |
| | GTCTTGATCATGGTCATGTCTATTTTGTTGTCCTCCCCAGAAAAGAGAAAGACCCCAGT |
| | TTTCATCGTCAACTCTGCCTCTATCATTTCCATGTGTATTAGAGCTATCTTGATGATTG |
| | TCAACTTGTGTTCTGAATCCTACTCTTTGGCTGTTATGTACGGTTTCGTCTTCGAATTG |
| | GTTGGTCAATACGTTCACGTTTTTGACATTTTGGTTATGATTATTGGTACCATCATCAT |
| | TATTACCGCTGAAGTTTCCATGTTGTTGCAAGTCAGAATTATTTGTGCTCACGACAGAA |
| | AGACTCAAAGAATTGTTACCTGTATCTCTTCTGGTTTATCCTTGATCGTCGTTGCCTTC |
| | TGGTTCACTGATATGTGTCAAGAAATTAAGTACTTGTTGTGGTTGACCCCATACAACAA |
| | CCACCAAATCTCTGGTTACTACTGGGTTTACTTCGTCGGTAAGATCTTGTTCGCCGTTT |
| | CCATTATGTTCCACTCTGCCGTCTTCTCCTACAAGTTGTTCCACGCTATCCAAATTAGA |
| | AAGAAGATTGGTCAATTCCCATTCGGTCCAATGCAATGTATTTTAATTATTTCCTGTCA |
| | ATGTTTGTTCGTTCCAGCTATTTTCACTATCATCGACTCTTTCATCCACACTTACGACG |
| | GTTTTTCCTCCATGACCCAATGTTTGTTGATCGTCTCTTTGCCATTGTCCTCCTTGTGG |
| | GCCTCTTCCACTGCTTTAAAGTTGCAATCTTTGAAGTCTACCACCTCTCCAGGTGACAC |
| | TACTCAAGTTTCCATTAGAGTCGACAGAACCTACGACATCAAGAGAATCCCAACTGAAG |
| | AATTGTCTTCTGTTGACGAAACCGAAATCAAGAAGTGGCCATAG |
| SjSte2 | ATGTACTCCTGGGACGAATTCAGATCCCCAAAGCAAGCTGAAGTTTTGAACCAAACCGT |
| | TACCTTGGAAACTATTGTTTCCACCATTCAATTGCCAATCTCTGAAATTGACTCCATGG |
| | AAAGAAACAGATTGTTGACCGGTATGACTGTCGCTGTTCAAGTTGGTTTAGGTTCCTTC |
| | ATTTTAGTTTTGATGTGTATTTTCTCTTCCTCTGAAAAGAGAAAGAAGCCAGTCTTCAT |
| | CTTCAACTTCGCTGGTAACTTGGTTATGACTTTGAGAGCTATTTTCGAAGTTATCGTTT |
| | TGGCTTCTAACAACTACTCTATCGCTGTTCAATACGGTTTCGCTTTTGCTGCCGTCAGA |
| | CAATACGTTCACGCCTTCAACATTATCATCTTGTTGTTGGGTCCATTCATCTTGTTCAT |
| | CGCTGAAATGTCTTTGATGTTGCAAGTTAGAATCATTTGTTCCCAACACAGACCAACTA |
| | TGATTACCACCACTGTTATCTCTTGTATTTTCACTGTTGTTACCTTGGCCTTCTGGATC |
| | ACCGACATGTCTCAAGAAATTGCTTACCAATTGTTCTTGAAAAACTACAACATGAAGCA |
| | AATTGTTGGTTACTCCTGGTTGTACTTTATCGCTAAGATCACCTTCGCTGCTTCCATTA |
| | TCTTCCATTCCTCCGTCTTCTCCTTCAAATTGATGCGTGCTATTTACATTCGTAGAAAG |
| | ATCGGTCAATTCCCATTCGGTCCAATGCAATGTATCTTCATTGTTTCCTGTCAATGTTT |
| | GATCGTTCCAGCTATTTTCACTTTGATCGATTCTTTCACCCACACTTACGATGGTTTCT |
| | CCTCCATGACTCAATGTTTGTTGATCATCTCCTTACCATTGTCTTCCTTGTGGGCCACC |
| | CACACCGCTCAAAAGTTGCAAACCATGAAGGATAACACTAACCCACCATCTGGTACCCA |
| | ATTAACCATCAGAGTTGATCGTACTTTCGACATGAAGTTCGTTTCCGACTCCTCTGACG |
| | GTTCTTTCACTGAAAAGACCGAAGAAACTTTGCCATAG |
| ScBar1 | ATGTCTGCAATTAATCATCTTTGTTTGAAACTTATTTTGGCGAGTTTCGCGATTATTAA |
| | CACCATTACTGCTTTAACAAACGATGGCACTGGTCACTTAGAATTCCTTTTACAACACG |
| | AAGAGGAGATGTATTACGCAACAACCTTAGATATAGGTACACCGTCCCAAAGTCTGACA |
| | GTGTTGTTTGATACCGGATCTGCCGATTTTTGGGTTATGGATTCTAGCAATCCCTTCTG |
| | CTTACCAAATTCAAATACGTCATCCTATTCAAACGCAACTTATAATGGCGAAGAAGTTA |
| | AGCCTTCAATTGATTGCAGGTCTATGAGTACTTATAATGAGCATAGATCTTCCACCTAC |
| | CAATATCTGGAAAATGGTAGGTTTTACATCACATATGCTGACGGAACATTTGCTGACGG |
| | TAGTTGGGGGACGGAAACTGTATCAATTAATGGAATTGACATCCCCAATATCCAGTTCG |
| | GAGTTGCCAAGTATGCTACGACACCCGTTAGTGGTGTTCTTGGAATTGGGTTTCCTAGA |
| | AGAGAGTCCGTTAAGGGCTATGAAGGTGCTCCTAATGAATATTATCCTAATTTTCCTCA |
| | GATTTTAAAAAGTGAAAAAATAATCGATGTGGTCGCGTATTCGCTGTTCTTAAACTCAC |
| | CTGATTCAGGTACTGGTTCGATTGTTTTTGGTGCCATTGATGAATCAAAGTTTTCTGGT |
| | GATTTGTTCACTTTCCCTATGGTAAATGAATATCCCACAATAGTCGACGCTCCTGCAAC |
| | TTTAGCAATGACTATACAAGGATTAGGTGCCCAAAACAAAAGTAGTTGTGAACATGAAA |
| | CGTTTACGACGACCAAGTATCCAGTTTTGTTGGACTCAGGAACCTCGCTATTGAATGCG |
| | CCCAAGGTCATAGCAGATAAAATGGCTTCTTTTGTAAATGCGTCCTATAGTGAAGAGGA |
| | AGGTATATATATATTAGACTGTCCAGTATCTGTAGGTGACGTGGAATACAATTTTGATT |
| | TCGGCGATTTGCAAATAAGTGTTCCACTGTCTAGTTTGATTTTAAGTCCCGAGACAGAA |
| | GGCAGCTATTGTGGGTTTGCGGTCCAGCCAACAAACGATTCGATGGTTCTGGGTGATGT |
| | GTTCCTGTCCTCTGCATACGTCGTATTCGATCTCGATAATTATAAGATATCTTTAGCAC |
| | AGGCAAATTGGAACGCAAGCGAAGTTTCGAAAAAGCTAGTAAATATTCAAACAGATGGG |
| | TCTATTTCAGGTGCCAAAATTGCTACAGCTGAACCCTGGTCCACCAATGAACCATTTAC |
| | AGTCACCTCTGACATTTATTCATCTACAGGCTGCAAGAGTAGGCCTTTTCTTCAATCAT |
| | CGACAGCCTCTTCGCTTATTGCAGAAACCAACGTACAAAGTCGCAACTGCTCTACGAAG |
| | ATGCCAGGCACTAGATCAACTACTGTCTTAAGTAAGCCTACTCAAAATAGTGCTATGCA |
| | TCAAAGTACAGGCGCTGTCACACAAACCTCAAATGAAACTAAATTAGAATTATCCTCGA |
| | CTATGGCAAATTCGGGCAGTGTCTCGCTTCCCACTTCGAATTCAATAGACAAAGAGTTC |
| | GAACATTCGAAATCTCAAACTACCAGCGATCCAAGTGTAGCAGAGCATTCTACGTTTAA |
| | CCAAACGTTTGTACATGAAACTAAATATCGGCCTACTCATAAGACAGTCATAACAGAAA |
| | CTGTCACGAAGTATTCTACAGTCTTAATAAATGTCTGTAAACCAACATATTAA |
| CaBar1 | ATGTTATTTACTATTCTTTCACTTCTTGTCCCATCCCTAGCCGTGGCCGTATCCAATAC |
| | CGGCGGTGCGGTGAAATTAGACCTTACGATATCCGATGAGCACATGTATTACATAGAGA |
| | ATTTAGCGTTAGGGACGCCGCCCCAGTTCATATCTAACGTTATAGTCGATTCAGGAAGT |
| | AGCGATCTTATGATAGTTGACTCCATATACAACTTCTCTGCGTCATCAAGTTTCTATAA |
| | CTCCAACCAGACCGCAATTATGAAATATGGATACGGGGGACAGTTCCCGGTATACTTCA |
| | TCAATGAAACTATTAGGAGTAACGACTGGAAACTGTCTAATCTATCTATGGGACTGGCC |
| | AATATTTCTGATATGGACAGTTTTTCCGGTATACTAGGCATTGGATTTACTCGTCAAGA |
| | ACTTTTTAAAACGAATTACAGTAACTTCCCCTATCTACTTAAAGACCAGGGTTATACGA |
| | AAAGTGTTCTATTCTCATTCAATGGTCAGGATCAGAACCCGTCCATTATCTTCGGAGGG |
| | ATAATTACGAACATCATAGACGGGCCTTTAGTAAGAGCGCCCTTTATCAAAGTGATATC |
| | CTTCATAAACCAGCTTGACTACTGGTTGATGCCAACGTTCACAGTAAATCAGATCAAGT |
| | TGGGAGACACCATCGTTAGCAACCAGAAGACGCTTTACCAAATCGATAGTGGCACTAAC |
| | GGTTTCGTCCCCCCAACTCCGGTCCTTAATAACATCCTTAAAATTTTAGGCGACGACTA |
| | CATCCAGGATGACAATGGCAATATATATTTCGACATCAAGTACATAGAGGGGTTAAATA |
| | TAACATTTTCAGTACAAGGGTACGACATTGGCTTCCAGCTGGTCGACATAGTCGGCGAT |
| | ACCATTGAAAGGAACTCAACTACATTCGTAGCCTTAAATGTAGCTTCATGCGATATAGG |
| | CTATAACGCCTATGAAGGTCTGCTGCCAAATTTGATTTTTAAGTACCACTATGCTATAT |
| | TTGACTACGACAACGCACAAATATATTTCGGTAAATATAAGAATTCTAACGGAGAGGCG |
| | AACGTCGTCGCTGTGGAGAATGGTTACCAGCTACCAGTACCGACAGTAGACGTGCCCGA |
| | TGTTGAGGACACTTATAGCGTTATATATATCGCGGAATCAGAAACCACGGTAACCGTTG |
| | CACCGACAGAGGTCCAAAGCACCTATTCATCCTTTTCTATCAGTGAGGGTACCTCTAGC |
| | AACCTAACGAGCGGCAGCACAAGTGATGATAAGATTACTTCCGTCACAAGCAACCCTCA |
| | GTATTGTTAA |
| SpSxa2 | ATGTTAAGTCTTTTCTTAAAGTCTTTGTTCGCAATTATTATCATTGAACTTACTATCAT |
| | CCACGCCCTTCCGACGTACACGGTGCACTGGAAGTGTAGCATACAACAAGCCAATACTT |
| | CCTCAGCATCCTCTAACCAGACCGTCCAGCCGCGTCAGCACGCAGCGCCGAGTAGCGAT |
| | CGTATAAAGTCCCTACCCGAATTTAAGGGATCACTGCCTGAGTTATACTCAGGTTACTT |
| | GGAGGCAAATTCTGACAAGTCACTATTTTACACATATGCGCCTGCAGTTGTAGACTCTG |
| | AAACGTTCATCGTGTGGTTGCAGGGCGGACCGGGCTGCGCGGGCACTCTAGGTTTCTTC |
| | AGTGAAAACGGCCCGATTGAGATTAGCCAGTCATCTCCTTCTCCTTCATTAAATCCTGA |
| | GAGTTGGACCAATTTCGCAAACATGTTGTGGCTGGACCAGCCTTTTGGTACTGGGTATA |
| | GCCAGGGGCAGGCGGCTTACACTACCACCATAGAGGAAGCCTCTAGCGACTTCGTCAAT |
| | GCCCTAAAATCTTTTTACCAAAAATTCCCGCATTTGATGAAAAAGAAGCTGTATCTTGT |
| | TGGGGAAAGCTATGGCAGTATATGGAGTGCCAATTTTGCGGAGGCCCTACTTAGCGAAC |
| | CAAGTCTAAATATCAATTTTATGGGTGTGGGTATTGTCAGCGGACTAACAGCCGATTAT |
| | GAGACTCAGGAACAAATAACGGCAAGTATATGGGTTGAGCATATATCCAAACTGGGTTA |
| | CTATTTTAACAATACCTCCTCTACCATATCAGAAGAGTTCAAGAAACGTAACAAAGAGT |
| | GTCAGTACGACTCCGTATTGAATAGATTAACGTTTCCCACCGAACAGTATCCCATATGG |
| | CGTCCTGAATACAATTTTTCAACCTCAACCAGTCTTAGAAAGAGGGAAGCACTGGATGG |
| | GGAAGATATCGGTAACGTTTTCAATTCCATCTCTGGTTGTGATCTGTATTCATTATCTA |
| | ACTTTCTACTGTACCTTGAAAACAGTTGCGTGATTACATACGACGTCAGTCTTGACTGC |
| | TCCTTCAATGAATATAACGATCCATTGATTACATACTTGAACAGAGAAGACGTCAGATC |
| | CTCCTTGCACGCAACCAAGGCCTCCACGGCGTTAACATCCGGAGAGGGTGTCTTCGCCG |
| | ATGGATGTAACTTCGATCTGTATAAGAAGATTGTCTCAAACAATGTCGAATCTGTGCTA |
| | GTAGAGATCATACCTCGTTTGACAGAGAAATATAAAGTCAGTTTCTTAGCAGGTGCTCT |
| | GGACCTACAAATTCTATGGACGGGGACTTTGTTAGCTCTACAGAACACTACATGGAACG |
| | GGTGGCAAGGATTTACACAATCTCCCGGCTCTCTGGAAACGACGAATGGTTTCACGCTG |
| | GATGAGAGAAATCTTGCGTTTACCCTAAGTAACAGCGTGGGCCATATGGCTCCAAGCAA |
| | GGATCCACAAATGGTGAGAGAATGGCTAGAGAACACCCTTCTTTACTAA |
| SoSxa2 | ATGAACTTAGCCGTCCATGTACTGCTGTTGTCTTTCTTGTTGTACACTGTTCACAGTTT |
| | CCCCGCATATCCGCCATATCCAATATATCCGAGATCATCTCTGAACTCAAGTAACGCCT |
| | CCAGCGTACCAATACCACAACCCTCATACAATTCATCTTCCAAGGCCATGAATCCTGAC |
| | CTGGTCACTAGCTTGCCAGAGTATTCAGGTAGTCTACCAGAATTATACAGCGGATACCT |
| | TAGCGCCGAGAACTTCAAGCAGATCTTTTATACTTTTGCACCTGCCATTACCCCGACTG |
| | ACAAATTCATAGTTTGGCTGCAAGGGGGTCCTGGGTGCGCAGGCACCTTAGGCTTCTTT |
| | TCCGAAAACGGTCCAATTATCTTGGACCCCAATCACCAAACTCCACAAGACAATCCCAA |
| | TAGCTGGACTCGTTTCGCAAACATGATGTGGATTGACCAACCCTTTGGAACAGGGTATT |
| | CTCAAGGCTCCCCATTGGCAAGTACAGTTTCAATCGCTACTTCAGATTTTATCCAAACT |
| | CTGAAGTCTTTTTACGCAAAATTCCCTGAGATGAAGGAAAAAGAGTTGTATCTTGTGGG |
| | CGAGTCCTATGGATCCGTGTGGGGCGCGTATTTTGCCCAAGCCTTGAGGTCCGATCCCG |
| | AGCTGCATGCTATACCTTTTGGGGGGCTAGGTATCATAAGCGGCCTTCTATCAGACTAT |
| | GAAACTCAGAATACTATAACAGCGTCAATGTGGCTTGATCATGTTAACAAAATGGGCTT |
| | GTATTATACTAATGAGTCCGCCGAGGTATCTACGTTTTTCGCTGCAAAGAATCAACAAT |
| | GTGGTTACGCAGAAGTCCTAGAGAGAATGCAATTCCCAGCCGAACAGACTCCGATACCT |
| | AAGCCGCGTGTCAATTTGACTAACTCTTTCTCAAAAAAGAAAAGGCAGGCCGACTCTGA |
| | CTCTGACTCCGGCAATATAAAGGAAGCATTTCAAATGATCACGGACTGTGATACTTATA |
| | CGTTAACGGACTTTCTTCTTTACCTGAAAAACCATTGTATGATAACATACGATATTACG |
| | CTTGATTGCTCATTCAATTCAAACAACGACCCTCTAACAACCTATCTAAATAGAAAAGA |
| | CGTACAGAAGCAACTGCACGCTATTCATGTCGAGAGCGCTTTAACATCCTCCAAAGGCA |
| | TCTTTGGTAATGGATGCAACTATGACGTCTATGGTGACATTGTCAAAAATGGCGCACCT |
| | TCAATACTGGAAGAAGCGATACCTGAGTTAGTACAGAACTATAAAGTCAGTTTCTTAGC |
| | CGGGGCCTTAGACTTTCAATTGCTTTGGACTGGTAGTTTGCTGGCACTACAAAACACGA |
| | CCTGGAACGGTTGGCAGGGTTTTCAAGAGATCCCTGACCTAAACTCCCCCTTTGGTTTT |
| | ACGCTTGACGAGCGTAATCTGGCGTTCACGCTTAGCAACAAGGTAGGACATATGGCCCC |
| | GAGCAAGGACCCCAGCATGGTAATAGATTGGCTGCAAAAAACCCTGCTGGGTTGA |
| SjSxa2 | ATGGTAGTTTTGCACAGATTGCTTTTAAGTTTGTGGCCCGCAGGAGCCATGTTGCTAGT |
| | GTCTTCATTTCTATTGAACCTGCCAACGACATCCGCGATTCCGATTTATAGCGATCCCC |
| | ACCAATGTTCACTTAGGGTTAGCGTTAGAACAGATGTCGAGGGGGAGCAACAACAGACT |
| | CAGGCAATGGGCTCTGTCGTTATTGCGTCAAATGTAACGTCATCCGACGCGCCGACCGA |
| | AAACCCCACGTCTAGTGCGCCAACGACTTTTAGTCACACTGTTAACAGTACATTTCCTA |
| | CCCATACGCCAACGACCAGTACCTCTACAAATACGCCCACGGACGCAAGAGTCAGCTCT |
| | TTACCTTTATTGAACGGCGACATGCCCGCACTGTATGCCGGCTACCTTAACGTAAGCGC |
| | AAGCTCAACGTCCCCACATTCTATTTTCTACGCGTATGCACCTTCTGAACAAGGGTCCG |
| | ATGATTTGATCGTATGGCTGCAAGGTGGGCCGGGATGCGCAGGCACTATTGGACTTTTC |
| | GCCGAAAATGGGCCGGTACGTTTCGGTCCTGGCGACAGCGCTCCCGTTCGTTCAGAACA |
| | CTCTTGGACTAGATACGCAGATGTACTTTATTTGGACCAACCATTCGGGACGGGATTTT |
| | CTTACGCTGTCACCGAAGCTGACTATACGACCACAATGGATGCGGCGTCTGCAGAATTC |
| | ATAGCATTTTTGGACGGATTCTTCGACGCATACCCAGATACAGCTTCTAAGAGGCTATT |
| | TTTCGTGGGGGAGTCTTACGGAAGTTTTTGGGGGGCCTACTTTGCCAGGGCCCTGGCAG |
| | ATAGACCTGACATTTATGATCGTATAGTTAGTGTGGGCATCGTGTCTGGACTATCAACT |
| | ACTCACGATACTCAGTACAGCGTATCTAACTCCTTATGGTTGTCTCACTTAAATAAGCT |
| | AGGCTATTATACCCAGGATAAAAGCACCTGGAATTCTTTTAGTAGAGTCAATGCACGTT |
| | GCGGTTACGACAAGGTAATGCAGGAGTTCACTTTCCCAGCGAGTCAGACGCCGCTTAGT |
| | AGTATATCTACGAGCACCTCAAGCTCAAGTACCAGTTCTGGAGCTGGAGCCGGAGCAAC |
| | GGTTTCTTCCTTGACCGATTCTTCAGGGAATGCAAGTTACACCAATTCTTCTAGCAGCG |
| | CTGAAGTAGACGTGTTTTCATACTTCCCCTCATGCGACACAAACGACGCAACGTACATG |
| | CTAATGTATTTGCAGAATACGTGTTTAATTTCTTATGACGTCTCTATCGACTGTACAAC |
| | TGCTTCCCAACTTACAGATCACTTAACGCCATACTTGAATAGGGCAGACGTTAGGGAGG |
| | CCTTGCATGTCCCCTCCTCTGTGGCTGCATTTAGCACGGCAAACGGTGTGTTTGGAGAC |
| | GGCTGCAACTATGATTTACTGCAGAAAATTAATACAGTTACCTACGAACCAGTTCTGAA |
| | ATCAGTCGTACCGAGCTTGATCGCCGATGGTAAAAGGGTGGCCTTTCTGTCAGGCGCAT |
| | TGGACTATCAGATTCCGTGGAGCGGAACCCTTTTGGCGATGCAAAATACTACTTGGGCC |
| | GGTTGGCAAGGATTTCAGACGGAGCCTGACTGGGACAACCCCGTAGGCTTTACGCTAAA |
| | CGAAAGAGGATTGAGTTTTACCTTACTTAATTCTGTGGGACACATGGCCTCAATGAATG |
| | ATCCAGACAGGGTGAACCGTTGGTTGCAGGAACAAGTCCTTTGA |

Yeast codon-optimized sequences without internal BsaI and NotI restriction sites.

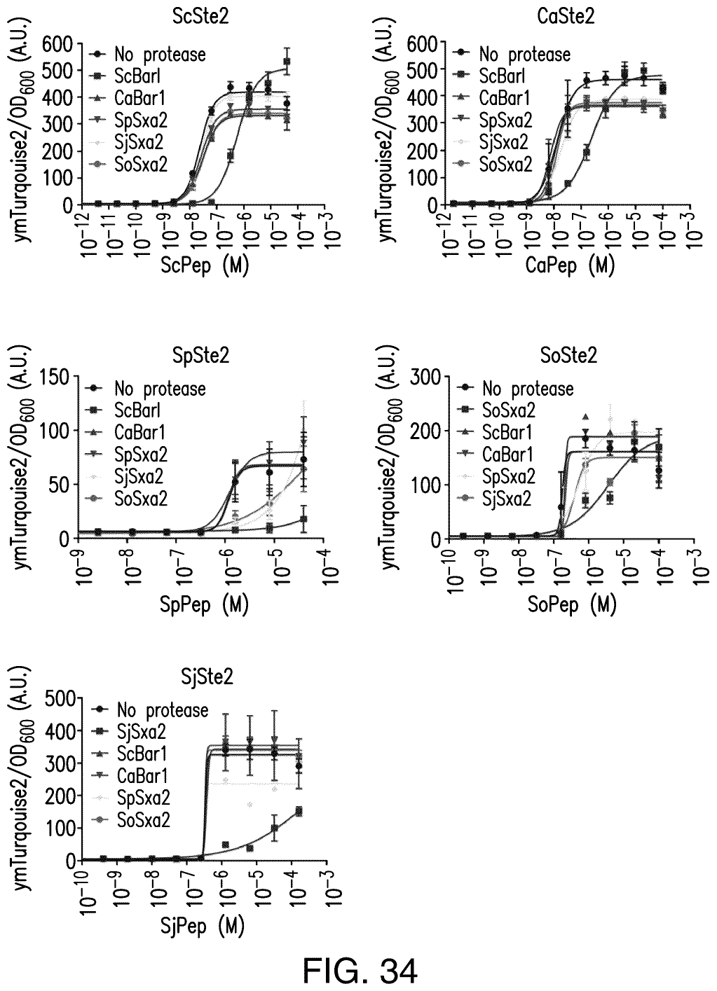

In order to validate the effect of peptide proteases on their complementary and non-complementary peptides (Table 19), from the parent strain yTC370 30 strains were engineered (Table 12). These strains all have the same peptide-inducible fluorescent readout (ymTurquoise2) and they have all possible combinations of complementary and non-complementary GPCRs and proteases, including strains which only have the GPCR and do not have any protease (ScSte2−ScBar1, ScSte2−CaBar1, ScSte2−SjSxa2, ScSte2−SoSxa2, ScSte2−SpSxa2, ScSte2, CaSte2−ScBar1, etc.). The fluorescence activation of each strain was measured after incubation at different concentrations of each complementary peptide. Dose-response curves were then generated from which the apparent shift in EC₅₀ towards higher peptide concentration were detected in an instance when the protease effectively cleaves the peptide and prevents it from activating the GPCR within a particular peptide concentration range (FIG. 34).

For Sc, Ca and Sj peptide/GPCR pairs, only their complementary proteases (ScBar1, CaBar1 and SjSxa2, respectively) shifted the apparent EC₅₀ for more than one order of magnitude. Other non-complementary protease combinations did not significantly affect apparent EC₅₀. For Sp and So pairs, their cognate proteases (SpSxa2 and SoSxa2, respectively) strongly shifted apparent EC₅₀. SpSxa2 practically completely attenuated the response with SpPep within measured concentration range and SoSxa2 shifted apparent EC₅₀ by one and a half orders of magnitude towards higher SoPep concentration. However, in Sp and So pairs, non-complementary proteases (SjSxa2 and SoSxa2, SjSxa2 and SpSxa2, respectively) also shifted apparent EC₅₀ between half and one order of magnitude, while ScBar1 and CaBar1 did not cleave SpPep and SoPep. For Sp and Sj peptide/GPCR pairs, the complementary proteases (SpSxa2 and SjSxa2, respectively) shifted the apparent EC₅₀ out of the measurable peptide concentration range (>100 μM).

TABLE 19

| Species (peptide) | Amino acid sequence |
|---|---|
| S. cerevisiae (ScPep) [14] | WHWLQLKPGQPMY |
| C. albicans (CaPep) [14] | GFRLTNFGYFEPG |
| S. pombe (SpPep) [14] | TYADFLRAYQSWNTFVNPDRPNL |
| S. octosporus (SoPep) [14] | TYEDFLRVYKNWWSFQNPDRPDL |
| S. japonicus (SjPep) [14] | VSDRVKQMLSHWWNERNPDTANL |

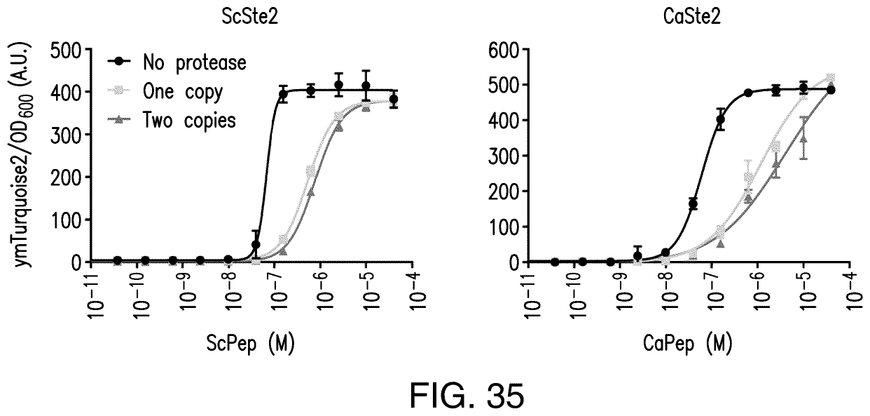

Additional integrated protease copy gives stronger shift in EC₅₀. Next, it was tested if integration of additional copies of ScBar1 and CaBar1 would lead to a higher shift in the apparent EC₅₀, as this would improve the working peptide concentration range where the activation difference between + protease and − protease strain could be distinguished (FIG. 30C). In Sc and Ca peptide/GPCR pair strains with their corresponding cognate proteases (yTC398 and yTC399, respectively), an additional copy of the strong constitutive protease expression cassette was integrated into HO locus (yBB01 and yBB02, respectively) (see Methods and Table 12). The dose-response curves in FIG. 35 show that the additional protease copy marginally shifts the apparent EC₅₀ compared to a single copy. Nonetheless, further experiments are done with two protease copies.

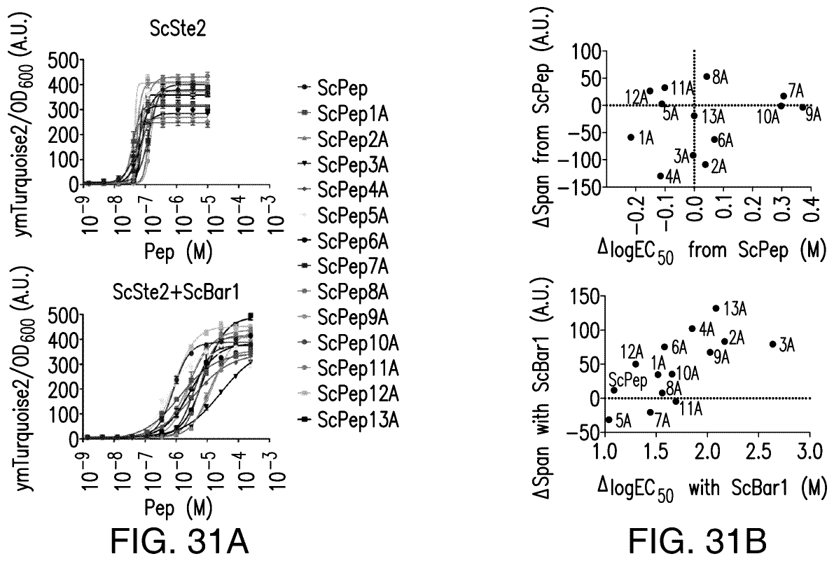

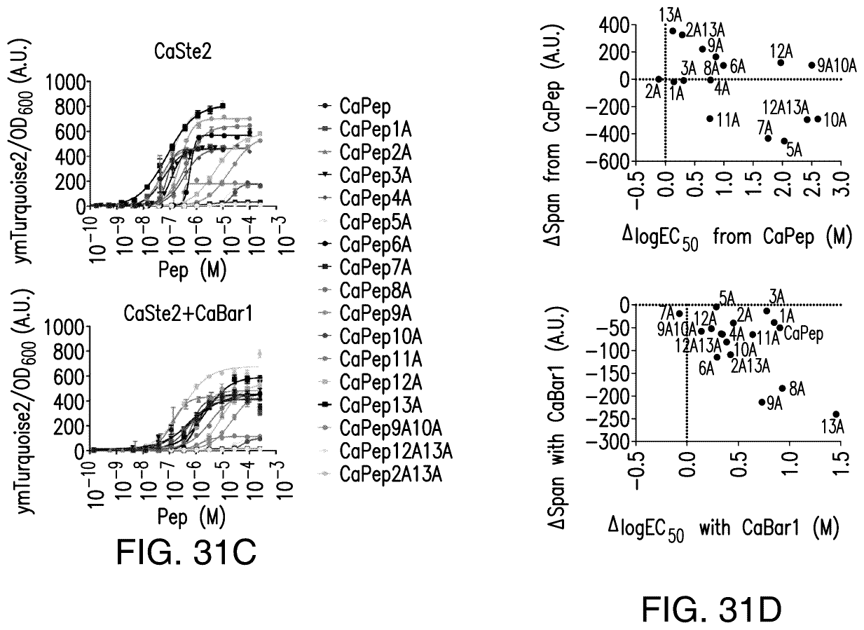

ScPep and CaPep Alanine scans show single amino acid-dependent activation by GFCR and cleavage by protease. A previous report of an Ala scan of the CaPep suggested that peptide/GPCR/protease systems may be able to distinguish single amino-acid changes in the peptide epitope [24]. To test this hypothesis, the effect of single-site Ala substitutions in ScPep and CaPep was systematically measured on the complementary GPCR activation and protease cleavage (FIG. 31) [25]. Dose-response activation of yTC391 (ScSte2) and yBB01 (ScSte2+ScBar1) was validated with ScPep and single Ala-substituted variants, and yTC384 (CaSte2) and yBB02 (CaSte2+CaBar1) with CaPep and Ala substituted variants by measuring ymTurquoise2 fluorescence intensity. Ala-scanned peptide sequences and measured Hill function parameters which are discussed below are in Tables 20 and 21.

TABLE 20

| | | | | | |
|---|---|---|---|---|---|
| | Peptide | Bottom | Top | LogEC₅₀ | Hill |
| Name | sequence | (A.U.) | (A.U.) | (M) | Slope |

ScSte2

| | | | | | |
|---|---|---|---|---|---|
| ScPep | WHWLQLKPGQPMY | 6.8 ± 3.0 | 380 ± 10 | −7.2 ± 0.1 | 2.0 ± 0.1 |
| ScPep1A | AHWLQLKPGQPMY | 6.4 ± 3.4 | 320 ± 10 | −7.4 ± 0.1 | 3.4 ± 0.8 |
| ScPep2A | WAWLQLKPGQPMY | 4.9 ± 2.1 | 270 ± 10 | −7.1 ± 0.1 | 3.7 ± 0.2 |
| ScPep3A | WHALQLKPGQPMY | 4.2 ± 1.6 | 280 ± 10 | −7.2 ± 0.1 | 2.5 ± 0.1 |
| ScPep4A | WHWAQLKPGQPMY | 4.6 ± 2.0 | 250 ± 10 | −7.3 ± 124.4 | N/A |
| ScPep5A | WHWLALKPGOPMY | 5.0 ± 2.3 | 380 ± 10 | −7.3 ± 0.1 | 3.4 ± 0.3 |
| ScPep6A | WHWLQAKPGOPMY | 3.9 ± 2.3 | 310 ± 10 | −7.1 ± 0.1 | 5.6 ± 0.7 |
| ScPep7A | WHWLQLAPGOPMY | 10 ± 4 | 400 ± 10 | −6.9 ± 0.1 | 2.6 ± 0.4 |
| ScPep8A | WHWLQLKAGQPMY | 4.5 ± 1.7 | 430 ± 10 | −7.1 ± 0.1 | 2.7 ± 0.1 |
| ScPep9A | WHWLQLKPAQPMY | 3.9 ± 2.1 | 370 ± 10 | −6.8 ± 0.1 | 5.0 ± 1.3 |
| ScPep10A | WHWLQLKPGAPMY | 4.9 ± 2.4 | 380 ± 10 | −6.9 ± 0.1 | 5.3 ± 3.7 |
| ScPep11A | WHWLQLKPGQAMY | 5.3 ± 3.7 | 410 ± 10 | −7.3 ± 0.1 | 4.6 ± 1.3 |
| ScPep12A | WHWLQLKPGQPAY | 5.9 ± 4.7 | 410 ± 10 | −7.3 ± 125.9 | N/A |
| ScPep13A | WHWLQLKPGQPMA | 4.9 ± 2.5 | 360 ± 10 | −7.2 ± 0.1 | 8.2 ± 3.1 |

ScSte2 + ScBar1

| | | | | | |
|---|---|---|---|---|---|
| ScPep | WHWLQLKPGQPMY | 5.3 ± 2.4 | 390 ± 10 | −6.1 ± 0.1 | 1.7 ± 0.1 |
| ScPep1A | AHWLQLKPGQPMY | −3.1 ± 5.8 | 350 ± 10 | −5.9 ± 0.1 | 0.76 ± 0.06 |
| ScPep2A | WAWLQLKPGQPMY | 5.9 ± 3.0 | 350 ± 10 | −5.0 ± 0.1 | 1.5 ± 0.1 |
| ScPep3A | WHALQLKPGQPMY | 6.4 ± 2.6 | 370 ± 20 | −4.6 ± 0.1 | 0.74 ± 0.05 |
| ScPep4A | WHWAQLKPGQPMY | −1.6 ± 2.9 | 340 ± 10 | −5.4 ± 0.1 | 0.72 ± 0.04 |
| ScPep5A | WHWLALKPGOPMY | 0.52 ± 8.7 | 340 ± 10 | −6.3 ± 0.1 | 1.2 ± 0.2 |
| ScPep6A | WHWLQAKPGQPMY | 0.38 ± 5.71 | 390 ± 10 | −5.5 ± 0.1 | 0.99 ± 0.10 |
| ScPep7A | WHWLQLAPGOPMY | 5.7 ± 1.3 | 380 ± 10 | −5.4 ± 0.1 | 1.7 ± 0.1 |
| ScPep8A | WHWLQLKAGQPMY | 4.6 ± 2.6 | 440 ± 10 | −5.6 ± 0.1 | 1.1 ± 0.1 |
| ScPep9A | WHWLQLKPAQPMY | 3.7 ± 2.1 | 440 ± 10 | −4.8 ± 0.2 | 1.2 ± 0.1 |
| ScPep10A | WHWLQLKPGAPMY | 8.3 ± 1.6 | 420 ± 10 | −5.2 ± 0.1 | 1.8 ± 0.1 |
| ScPep11A | WHWLQLKPGQAMY | 8.0 ± 2.5 | 410 ± 10 | −5.6 ± 0.1 | 1.5 ± 0.1 |
| ScPep12A | WHWLQLKPGQPAY | 2.5 ± 2.9 | 450 ± 10 | −6.0 ± 0.1 | 1.3 ± 0.1 |
| ScPep13A | WHWLQLKPGQPMA | 4.6 ± 2.5 | 490 ± 10 | −5.1 ± 0.1 | 1.2 ± 0.1 |

Best-fit ± standard error Hill function parameters from ScSte2 alanine scan.

TABLE 21

| | | | | | |
|---|---|---|---|---|---|
| | Peptide | Bottom | Top | LogEC₅₀ | Hill |
| Name | sequence | (A.U.) | (A.U.) | (M) | Slope |

CaSte2

| | | | | | |
|---|---|---|---|---|---|
| CaPep | GFRLTNFGYFEPG | −1.9 ± 10 | 460 ± 10 | −7.2 ± 0.1 | 1.3 ± 0.2 |
| CaPep1A | AFRLTNFGYFEPG | 14 ± 7 | 460 ± 9 | −7.1 ± 0.1 | 3.0 ± 0.4 |
| CaPep2A | GARLTNFGYFEPG | 0.97 ± 10 | 460 ± 20 | −7.4 ± 0.1 | 1.4 ± 0.3 |
| CaPep3A | GFALTNFGYFEPG | 5.7 ± 3.4 | 460 ± 10 | −6.9 ± 0.1 | 2.0 ± 0.1 |
| CaPep4A | GFRATNFGYFEPG | 3.4 ± 4.9 | 460 ± 10 | −6.5 ± 0.1 | 1.3 ± 0.1 |
| CaPep5A | GFRLANFGYFEPG | 3.8 ± 0.3 | 15 ± 0.5 | −5.2 ± 0.1 | 2.7 ± 0.7 |
| CaPep6A | GFRLTAFGYFEPG | 4.7 ± 2.8 | 570 ± 10 | −6.2 ± 0.1 | 4.4 ± 0.4 |
| CaPep7A | GFRLTNAGYFEPG | 3.4 ± 0.2 | 34 ± 0.4 | −5.5 ± 0.1 | 1.4 ± 0.1 |
| CaPep8A | GFRLTNFAYFEPG | 15.3 ± 21 | 640 ± 20 | −6.4 ± 0.1 | 1.3 ± 0.3 |
| CaPep9A | GFRLTNFGAFEPG | 17 ± 6 | 700 ± 10 | −6.6 ± 0.1 | 1.9 ± 0.1 |
| CaPep10A | GFRLTNFGYAEPG | 5.6 ± 1.9 | 180 ± 10 | −4.6 ± 0.1 | 2.8 ± 0.5 |
| CaPep11A | GFRLTNFGYFAPG | 6.1 ± 3.9 | 180 ± 10 | −6.5 ± 120 | N/A |
| CaPep12A | GFRLTNFGYFEAG | −1.7 ± 3.4 | 580 ± 10 | −5.3 ± 0.1 | 0.95 ± 0.04 |
| CaPep13A | GFRLTNFGYFEPA | −4.4 ± 8.1 | 820 ± 10 | −7.1 ± 0.1 | 0.81 ± 0.04 |
| CaPep9A10A | GFRLTNFGAAEPG | 2.9 ± 2.2 | 570 ± 10 | −4.7 ± 0.1 | 0.99 ± 0.04 |
| CaPep12A13A | GFRLTNFGYFEAA | 4.9 ± 0.5 | 170 ± 10 | −4.8 ± 0.1 | 2.8 ± 0.1 |
| CaPep2A13A | GARLINFGYFEPA | 1.5 ± 6.2 | 790 ± 10 | −7.0 ± 0.1 | 1.2 ± 0.1 |

CaSte2 + CaBar1

| | | | | | |
|---|---|---|---|---|---|
| CaPep | GFRLTNFGYFEPG | 5.6 ± 17.4 | 420 ± 20 | −6.3 ± 0.1 | 0.87 ± 0.17 |
| CaPep1A | AFRLTNFGYFEPG | 4.5 ± 14.4 | 410 ± 10 | −6.2 ± 0.1 | 1.1 ± 0.22 |
| CaPep2A | GARLTNFGYFEPG | 12 ± 13 | 430 ± 10 | −6.9 ± 0.1 | 1.6 ± 0.3 |
| CaPep3A | GFALTNFGYFEPG | 14 ± 11 | 460 ± 10 | −6.1 ± 0.1 | 0.74 ± 0.08 |
| CaPep4A | GFRATNFGYFEPG | 11 ± 8 | 410 ± 8.4 | −6.1 ± 0.1 | 2.4 ± 0.4 |
| CaPep5A | GFRLANFGYFEPG | 6.4 ± 0.2 | 13 ± 1 | −4.9 ± 0.1 | 3.3 ± 0.1.3 |
| CaPep6A | GFRLTAFGYFEPG | 0.83 ± 6.2 | 450 ± 10 | −5.9 ± 0.1 | 1.7 ± 0.2 |
| CaPep7A | GFRLTNAGYFEPG | 5.1 ± 0.2 | 17 ± 1 | −5.6 ± 0.1 | 2.2 ± 0.4 |
| CaPep8A | GFRLTNFAYFEPG | 13 ± 8 | 460 ± 10 | −5.5 ± 0.1 | 1.0 ± 0.1 |
| CaPep9A | GFRLTNFGAFEPG | 10 ± 7 | 480 ± 10 | −5.9 ± 0.1 | 1.7 ± 0.2 |
| CaPep10A | GFRLTNFGYAEPG | 6.0 ± 0.4 | 98 ± 1 | −4.2 ± 0.1 | 2.6 ± 0.13 |
| CaPep11A | GFRLTNFGYFAPG | 5.4 ± 2.2 | 120 ± 10 | −5.8 ± 0.1 | 1.5 ± 0.2 |
| CaPep12A | GFRLTNFGYFEAG | 2.2 ± 4.8 | 540 ± 10 | −5.0 ± 0.1 | 1.1 ± 0.1 |
| CaPep13A | GFRLTNFGYFEPA | 14 ± 6 | 590 ± 10 | −5.7 ± 0.1 | 0.94 ± 0.06 |
| CaPep9A10A | GFRLTNFGAAEPG | 3.5 ± 3.8 | 510 ± 20 | −4.6 ± 0.1 | 1.0 ± 0.1 |
| CaPep12A13A | GFRLTNFGYFEAA | 5.6 ± 0.5 | 11−0 ± 10 | −4.5 ± 0.1 | 1.8 ± 0.1 |
| CaPep2A13A | GARLTNFGYFEPA | −2.4 ± 22.85 | 680 ± 20 | −6.5 ± 0.1 | 0.86 ± 0.10 |

Best-fit ± standard error Hill function parameters from CaSte2 alanine scan.

For Sc peptide/GPCR pair (FIG. 31A-B), the original ScPep and single-site Ala variants activated cognate GPCR with EC₅₀ ranging between 40 and 160 nM. The maximum fluorescent intensity of the variants was no more than 30% value of the original ScPep. When ScBar1 protease was co-expressed, different GPCR responses compared to the initial system were observed. Original ScPep and all variant peptides had shifted apparent EC₅₀ to higher peptide concentrations compared to system without ScBar1. The shift ranged between one to two and a half orders of magnitude towards higher peptide concentration. Interestingly, the maximal activation increased for the majority of the variants. Introduction of ScBar1 also affected the Hill slope values. In sensors, the Hill slope describes their ligand concentration operational range with higher Hill slope values meaning wider operational concentration range and lower Hill slope values meaning lower operational concentration range where the sensor responds in a switch-like manner [22]. ScBar1 significantly decreased Hill slope values for ScPep and all of its variants where dose-response curves have more gradual response over wider concentration range.

For Ca peptide/GPCR pair (FIG. 31C-D) there is a greater disparity in single amino acid effects in GPCR activation. In a system without the CaBar1 protease, mutants CaPep5A and 7A are practically non-responsive, while CaPep10A, 12A, 9A10A and 12A13A have more than two orders of magnitude higher EC₅₀ compared to the native CaPep. CaPep11A has the maximal activation reduced by 50% compared to the native CaPep. GPCR activation in the presence of CaBar1 for other functional mutants shows shifts in EC₅₀ between zero to one order of magnitude on top of already shifted EC₅₀ due to peptide sequence. Similar to Sc Ala scan, introduction of CaBar1 led to decrease in Hill slope values for the CaPep and the majority of its mutants. Based on these results, native CaPep and CaPep mutants 2A, 13A and 2A13A were selected as suitable candidates for further validation of a biosensor for peptide single amino acid differentiation (CaPep vs. 2A and 13A vs. 2A13A) in visible readout strains.

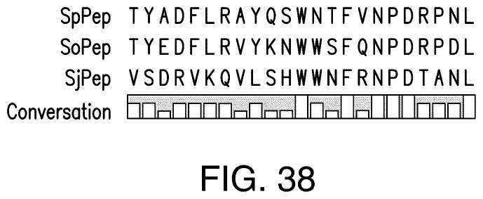

Our results confirm the specificity of fungal pheromone proteases to their native pheromone peptides as discussed in previous studies (FIG. 34) [26]. The cross-cleavage of noncognate peptides only significantly activated receptors with similar cognate peptides (FIG. 38), evident by the cross-cleavage results of SoSxa2/SjSxa2 for SpPep and SjSxa2/SpSxa2 for SoPep. The alanine scans of both ScPep and CaPep demonstrated the selectivity of cognate proteases. In the ScPep alanine scan, all point mutations were tolerated, but the addition of ScBar1 protease shifted the dose-response curves by more than an order of magnitude. On the other hand, the CaPep alanine scan showed that both N- and C-terminal mutations were less prone to CaBar1 cleavage. Additionally, a CaPep mutation in position 2 rendered the peptide non-cleavable and retained that property in the CaPep2A13A variant of the cleavable CaPep13A (FIG. 31).

It was previously showed that many fungal GPCRs expressed in yeast are highly orthogonal to their cognate peptides [14]. However, in agreement with previous studies, the GPCR was still activated after the introduction of most point mutations throughout both ScPep and CaPep. Flanagan et al. showed that substitutions in ScPep in positions 1, 5 and 7 retained its pheromone activity in growth inhibition assay [27]. Siegel et al. showed that substitutions in ScPep in positions 12 and 12+13 showed similarly strong growth inhibition as the native ScPep, while substitution in position 1 led to lower inhibition [28]. In the most extensive study on mutant ScPep activity, Abel et al. performed a systematic alanine scan where all residues in ScPep were replaced and showed that all L-enantiomeric mutants were able to illicit growth inhibition in ScPep-responsive yeast strain [29].

Regarding CaPep, Alby & Bennett demonstrated diminished peptide-induction of GFP expression in a di-alanine scan [24]. A similar result was observed with two disubstituted peptides: CaPep9A10A and CaPep12A13A. As expected, the double alanine substitutions produced lower activity than a single alanine substitution. Yet, it was also found that neighboring amino acids within CaPep can yield significant differences in activation.

Preincubation of lycopene readout strains improves cleavage efficiency of protease-sensitive peptides. In order to demonstrate that variant detection and differentiation can be achieved in the live yeast diagnostic with readout visible to the naked eye, biosensing strains were engineered which have carotenoid pigment lycopene as a readout, instead of fluorescent protein. Their activation was tested with selected peptides from the CaPep Ala scan (CaPep, CaPep2A, CaPep13A and CaPep2A13A). The strain dose-response activation was measured by absorbance and visual inspection of the wells after incubation with each of the selected peptides at different concentrations. Additionally, it was then validated if preincubation of the strains before adding the peptides would more effectively shift apparent EC₅₀ in the strain expressing the protease in order to increase working concentration range. It was hypothesized that with preincubation, the secreted protease concentration would build up in the medium and have greater catalytic activity once the peptide is added, thus shifting EC₅₀ more strongly.

First, based on the previously reported yeast biosensor [7], a parent strain yTC412 analogous to yTC370 was engineered which has integrated all relevant genes in lycopene biosynthetic pathway. This strain is only missing GPCR and protease component which are assembled using GGA and integrated using acceptor integration plasmid. Lycopene is a carotenoid pigment naturally produced by bacteria and plants. Lycopene can be biosynthesized in yeast from native precursor farnesyl pyrophosphate by introducing only three additional genes: geranylgeranyl diphosphate synthase (CrtE), phytoene synthase (CrtB) and lycopene synthase (CrtI).

In order to engineer lycopene parent strain yTC412, the first two biosynthetic genes CrtE and CrtB under strong constitutive promoters and two copies of the last gene lycopene synthase (CrtI) under control of peptide-inducible promoter were integrated into yTC370. Additionally, a copy of the endogenous flavin adenine dinucleotide synthase (FAD1) was integrated in order to achieve faster color development (see Methods and Table 14). This strain constitutes a complete peptide-inducible lycopene biosynthetic pathway and the final component needed in order to achieve peptide biosensing is the receptor and protease. CaSte2 without CaBar1 or with two copies of CaBar1 was integrated into this parent biosensor strain (yTC646 and yTC682, respectively).

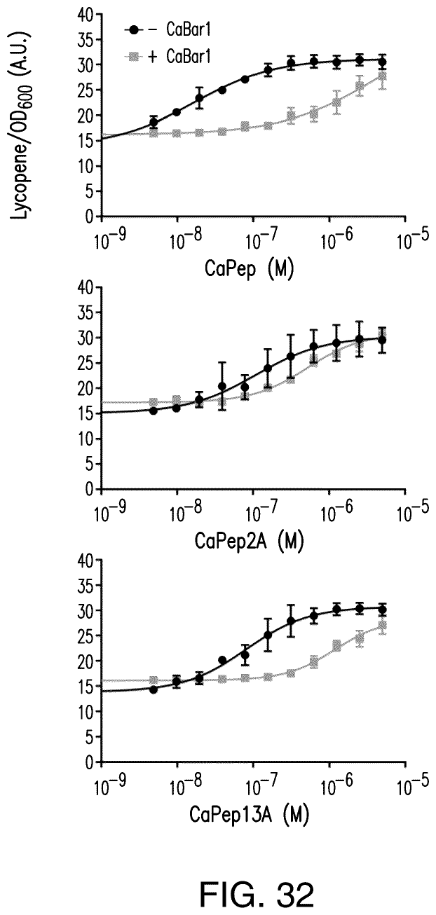

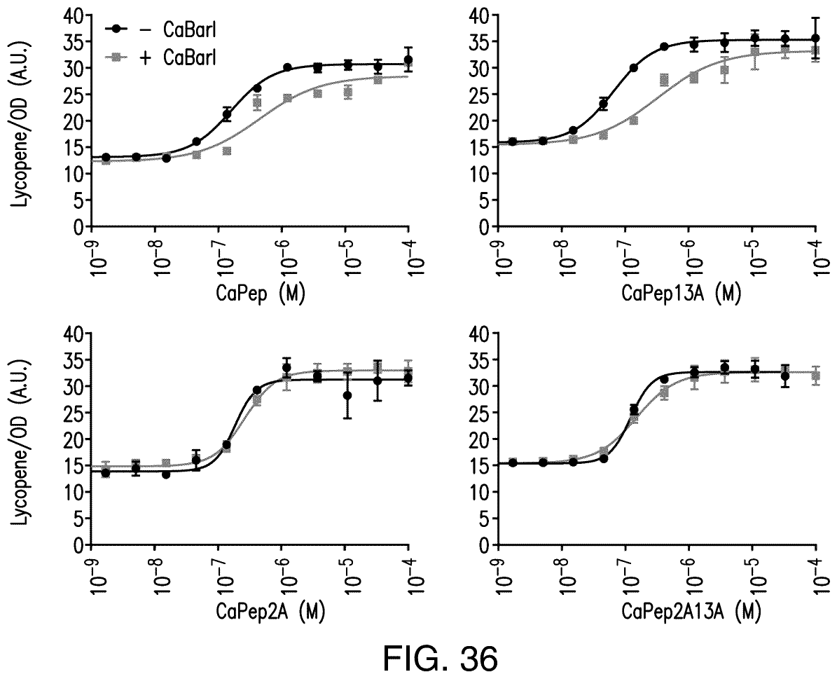

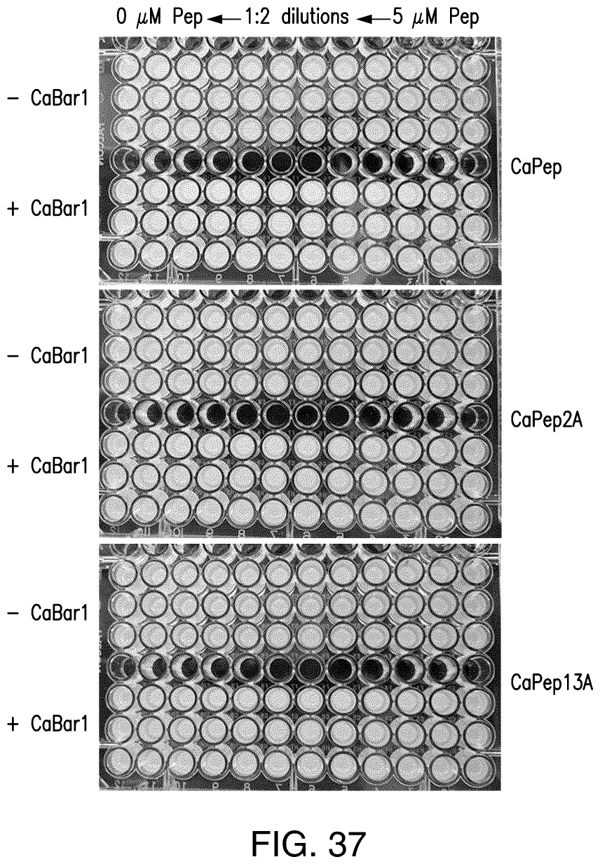

Next, lycopene strains activation and color development were validated in liquid culture with four selected peptides (CaPep, CaPep2A, CaPep13A, CaPep2A13A). The dose-response graphs in FIG. 36 show that CaPep2A and CaPep2A13A (non-cleavable variants) had similar response in both −CaBar1 and +CaBar1 strains with EC₅₀s between 120 and 250 nM and the same maximal activation intensity. On the other hand, CaPep and CaPep13A (cleavable variants) had EC₅₀ shifted by at least three-fold in +CaBar1 strain (from 160 to 450 nM and from 66 to 330 nM, respectively). In order to make the shift more prominent and to expand working concentration range where single amino acid difference can be distinguished, biosensor strains were preincubated before adding the peptides. This setup led to around two orders of magnitude shifted response in +CaBar1 lycopene strain towards higher concentration when activated with CaPep and CaPep13A (160 nM to ˜4,000 nM and 80 nM to 1,000 nM, respectively) (FIG. 32 and FIG. 37).

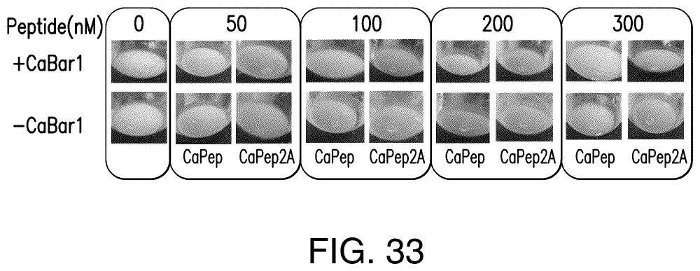

Living Yeast Biosensor Pius Protease can distinguish variants in product prototype. Encouraged by these results, the color change in concentrated yeast pellets was visually inspected in order to reconstitute the biosensor setup depicted in FIG. 30D. Ca-responsive biosensing strains were incubated with or without the CaBar1 protease (yTC682 and yTC646, respectively) in transparent culture tubes for one hour and then added either CaPep or CaPep2A in nanomolar concentration range. The cultures were incubated for another seven hours and then centrifuged in order to observe the color change in the yeast pellets. Using this assay, a robust orange color development was observed in −CaBar1 strain with both tested peptides, but not in +CaBar1 strain with CaPep2A in 50-300 nM concentration range (FIG. 33).

### Discussion

These results show that engineered S. cerevisiae, expressing promiscuous peptide GPCR and native proteases, provide feasible peptide detection and differentiation in the nanomolar range. a novel method for differentiation of peptide antigens based on (1) semi-specific activation of GPCR and (2) specific peptide cleavage by a self-expressed protease was established. Altogether, the method established a living yeast diagnostic prototype that distinguishes single amino acid mutant variants.

Nucleic-acid base diagnostics already employ kinetic proofreading to distinguish similar antigen variants [30]. In this example, it was shown that kinetic proofreading can be applied to the living yeast diagnostic, where the pheromone proteases provide a sink for variant pathways. This proof-of-concept application of kinetic proofreading toward peptide targets can be more broadly applied to antigen-based diagnostics to provide variant differentiation.

Variant detection through kinetic proofreading was simple to integrate into the living yeast biosensor. In a similar manner, this principle can be expanded to other engineered functionalities in living cells, such as variant sensing and responding by secretion of appropriate therapeutic. Recent advancements in synthetic biology provide tools for convenient integration of diagnostic and theranostic modules into engineered microorganisms [31-33]. In other words, living biosensors can adapt to novel challenges and stringent requirements for pathogen detection.

Living biosensors offer distinct advantages of rapid scalability and ease-of-use compared to conventional diagnostics, allowing for swiftly deployable surveillance of emerging pathogens on a global scale [34]. Despite advancement and momentum in the field of biosensor technology, living diagnostic biosensors are not yet available for detection of the variants and point mutations within the same pathogen. Expansive knowledge stemming from fundamental understanding of biological processes can fuel the innovation and applications in the field of synthetic biology.

In conclusion, the principles of kinetic proofreading were applied to the living yeast biosensors to detect peptide variants. This prototype can be expanded to detecting novel variants of pathogenic antigens, and shows the utility of living biosensors.

### REFERENCES

[1] T. R. Mercer and M. Salit, Testing at Scale during the COVID-19 Pandemic, Nat. Rev. Genet. 22, 415 (2021).
[2] P. Rajapaksha, A. Elbourne, S. Gangadoo, R. Brown, D. Cozzolino, and J. Chapman, A Review ofMethods for the Detection of Pathogenic Microorganisms, Analyst 144, 396 (2019).
[3] T. A. Alandijany, S. A. El-Kafrawy, A. M. Tolah, S. S. Sohrab, A. A. Faizo, A. M. Hassan, T. L. Alsubhi, N. A. Othman, and E. I. Azhar, Development and Optimization of In-House ELISA for Detection of Human IgG Antibody to SARS-CoV-2 Full-length Spike Protein, Pathogens 9, 1 (2020).
[4] C. C. Kerr, D. Mistry, R. M. Stuart, K. Rosenfeld, G. R. Hart, R. C. Núñflez, J. A. Cohen, P. Selvaraj, R. G. Abeysuriya, M. Jastrzebski, L. George, B. Hagedorn, J. Panovska-Griffiths, M. Fagalde, J. Duchin, M. Famulare, and D. J. Klein, Controlling COVID-19 via Test-Trace-Quarantine, Nat. Commun. 12, 1 (2021).
[5] M. Pavelka, K. Van-Zandvoort, S. Abbott, K. Sherratt, M. Majdan, P. Jarčuška, M. Krajči, S. Flasche, and S. Funk, The Impact of Population-Wide Rapid Antigen Testing on SARS-CoV-2 Prevalence in Slovakia, Science (80.). 372, 635 (2021).
[6] T. Ripa and P. Nilsson, A Variant of Chlamydia Trachomatis with Deletion in Cryptic Plasmid: Implications for Use of PCR Diagnostic Tests, Euro Surveill 11, 3076 (2006).
[7] N. Ostrov, M. Jimenez, S. Billerbeck, J. Brisbois, J. Matragrano, A. Ager, and V. W. Cornish, A Modular Yeast Biosensor for Low-Cost Point-of-Care Pathogen Detection, Sci. Adv. 3, e1603221 (2017).
[8] N. Barkai, M. D. Rose, and N. S. Wingreen, Protease Helps Yeast Find Mating Partners, Nature 396, 422 (1998).
[9] M. Jin, B. Errede, M. Behar, W. Mather, S. Nayak, J. Hasty, H. G. Dohlman, and T. C. Elston, Yeast Dynamically Modify Their Environment to Achieve Better Mating Efficiency, Sci. Signal. 4, ra54 (2011).
[10] J. J. Hopefield, Kinetic Proofreading: A New Mechanism for Reducing Errors in Biosynthetic Processes Requiring High Specificity, Proc. Natl. Acad. Sci. U.S.A. 71, 4135 (1974).
[11] T. W. McKeithan, Kinetic Proofreading in T-Cell Receptor Signal Transduction, Proc. Natl. Acad. Sci. U.S.A. 92, 5042 (1995).
[12] N. S. Olesnicky, A. J. Brown, Y. Honda, S. L. Dyos, S. J. Dowell, and L. A. Casselton, Self-Compatible B Mutants in Coprinus With Altered Pheromone-Receptor Specificities, Genetics 156, 1025 (2000).
[13] T. J. Fowler, M. F. Mitton, L. J. Vaillancourt, and C. A. Raper, Changes in Mate Recognition Through Alterations of Pheromones and Receptors in the Multisexual Mushroom Fungus Schizophyllum Commune, Genetics 158, 1491 (2001).
[14] S. Billerbeck, J. Brisbois, N. Agmon, M. Jimenez, J. Temple, M. Shen, J. D. Boeke, and V. W. Cornish, A Scalable Peptide-GPCR Language for Engineering Multicellular Communication, Nat. Commun. 9, 1 (2018).
[15] M. Grundmann and E. Kostenis, Temporal Bias: Time-Encoded Dynamic GPCR Signaling, Trends Pharmacol. Sci. 38, 1110 (2017).
[16] R. Daniel Gietz and R. H. Schiestl, High-Efficiency Yeast Transformation Using the LiAc/SS Carrier DNA/PEG Method, Nat. Protoc. 2, 31 (2007).
[17] M. E. Lee, W. C. Deloache, B. Cervantes, and J. E. Dueber, A Highly Characterized Yeast Toolkit for Modular, Multipart Assembly, ACS Synth. Biol. 4, 975 (2015).
[18] N. Ostrov, M. Jimenez, S. Billerbeck, J. Brisbois, J. Matragrano, A. Ager, and V. W. Cornish, A Modular Yeast Biosensor for Low-Cost Point-of-Care Pathogen Detection, Sci. Adv. 3, e1603221 (2017).
[19] V. L. Mackay, S. K. Welch, M. Y. Insley, T. R. Manneyt, J. Holly, G. C. Saari, and M. L. Parker, The Saccharomyces Cerevisiae BARJ Gene Encodes an Exported Protein with Homology to Pepsin, Biochemistry 85, 55 (1988).
[20] D. Schaefer, P. C\check{o}te, M. Whiteway, and R. J. Bennett, Barrier Activity in Candida Albicans Mediates Pheromone Degradation and Promotes Mating, Eukaryot. Cell 6, 907 (2007).
[21] Y. Imai and M. Yamamoto, Schizosaccharomyces Pombe Sxa1+ and Sxa2+ Encode Putative Proteases Involved in the Mating Response, Mol. Cell. Biol. 12, 1827 (1992).
[22] A. M. William Shaw, H. Yamauchi, J. Mead, M. Wigglesworth, G. Ladds, T. Ellis Correspondence, W. M. Shaw, G.-O. F. Gowers, D. J. Bell, N. Larsson, and T. Ellis, Engineering a Model Cell for Rational Tuning of GPCR Signaling, Cell 177, 1 (2019).
[23] M. E. Lee, W. C. Deloache, B. Cervantes, and J. E. Dueber, A Highly Characterized Yeast Toolkit for Modular, Multipart Assembly, ACS Synth. Biol. 4, 975 (2015).
[24] K. Alby and R. J. Bennett, Interspecies Pheromone Signaling Promotes Biofilm Formation and Same-Sex Mating in Candida Albicans, Proc. Natl. Acad. Sci. U. S. A. 108, 2510 (2011).
[25] B. C. Cunningham and J. A. Wells, High-Resolution Epitope Mapping of HGH-Receptor Interactions by Alanine-Scanning Mutagenesis, Science (80.). 244, 1081 (1989).
[26] S. K. Jones, S. C. Clarke, C. S. Craik, and R. J. Bennett, Evolutionary Selection on Barrier Activity: Bar1 Is an Aspartyl Protease with Novel Substrate Specificity, Am. Soc. Microbiol. 6, e01604 (2015).
[27] G. Houen, O. Nielsen, and C. Flanagan, Structural Requirements for α-Mating Factor Activity, FEBS Lett. 391, 162 (1996).
[28] E. G. Siegel, R. GOnther, H. Schtafer, U. R. Fölsch, and W. E. Schmidt, Characterization of Novel Peptide Agonists of the α Mating Factor of Saccharomyces Cerevisiae, Anal. Biochem. 275, 109 (1999).
[29] M. G. Abel, Y. L. Zhang, H. F. Lu, F. Naider, and J. M. Becker, Structure-Function Analysis of the Saccharomyces Cerevisiae Tridecapeptide Pheromone Using Alanine-Scanned Analogs, J. Pept. Res. 52, 95 (1998).
[30] O. Higgins and T. J. Smith, Loop-Primer Endonuclease Cleavage-Loop-Mediated Isothermal Amplification Technology for Multiplex Pathogen Detection and Single-Nucleotide Polymorphism Identification, J. Mol. Diagnostics 22, 640 (2020).
[31] W. C. Ruder, T. Lu, and J. J. Collins, Synthetic Biology Moving into the Clinic, Science (80.). 333, 1248 (2011).
[32] J. K. Rogers, N. D. Taylor, and G. M. Church, Biosensor-Based Engineering of Biosynthetic Pathways, Curr. Opin. Biotechnol. 42, 84 (2016).
[33] Y. Wu, C.-W. Wang, D. Wang, and N. Wei, A Whole-Cell Biosensor for Point-of-Care Detection of Waterborne Bacterial Pathogens, ACS Synth. Biol. 10, 333 (2021).
[34] B. J, K. Chanda, and B. MM, Biosensors for Pathogen Surveillance, Environ. Chem. Lett. 16, 1325 (2018).
[35] M. Jin, B. Errede, M. Behar, W. Mather, S. Nayak, J. Hasty, H. G. Dohlman, and T. C. Elston, Yeast Dynamically Modify Their Environment to Achieve Better Mating Efficiency, Sci. Signal. 4, ra54 (2011).
[36] G. Ladds and J. Davey, Sxa2Is a Serine Carboxypeptidase That Degrades Extracellular P-Factor in the Fission Yeast Schizosaccharomyces Pombe, Mol. Microbiol. 36, 377 (2000).
[37] A. C. Burkholder and L. H. Hartwell, The Yeast α-Factor Receptor: Structural Properties Deduced from the Sequence of the STE2 Gene, Nucleic Acids Res. 13, 8463 (1985).

### Example 16: Modular Electrochemical Interfacing Language for Living Yeast Biosensors

This example describes creating a scalable interfacing language based on genetically encodable redox-active peptides in order to overcome current limitations in redox potential resolution. The peptides investigated here aim to have unique electrochemical signatures (redox potentials) which can be directly de-coded by a solid-state device based on cyclic voltammetry, square-wave voltammetry or chronopotentiometry. Peptides are information dense and can encode a tunable range of voltage channels—they may be disulfide-linked cyclic, metal-binding or containing a genetically incorporated unnatural amino acid (UAA). Peptides can be naturally synthesized by living cells, and each secreted peptide constitutes a unique communication channel conveying discrete information between the biological and the solid-state system.

### Materials and Methods

### Peptide Disulfide Bond Oxidation and Reduction:

Oxidation—Peptide disulfide bond oxidation was done by modifying the protocol from [267]. Stock peptide solution was serially diluted in water in order to prepare different peptide concentrations for dose-response validation. Then, 100% dimethyl sulfoxide (DMSO) was added to each peptide dilution at final concentration of 12.2%. Peptides were incubated on benchtop for 1.5 h and then added to cells with final DMSO concentration at 1.22%.

Reduction—Peptide disulfide bond reduction was done by modifying the protocol from [268]. Stock peptide solution was serially diluted in water in order to prepare different peptide concentrations for dose-response validation. Then, 30 mM Dithiothreitol (DTT) was added to each peptide dilution at final concentration of 3.65 mM. Peptides were incubated on benchtop for 1.5 h and then added to cells with final DTT concentration at 0.365 mM.

### Cyclic Voltammetry:

Peptides used for cyclic voltammetry measurements were diluted to appropriate concentration in appropriate electrolyte solution (details noted in Figure captions). Measurements were done using CHI 760D potentiostat (CH Instruments, Inc., Austin, TX) and glassy carbon electrode with the following settings:
Init E (V)=0
High E (V)=0.6
Low E (V)=−0.2
Final E (V)=0
Init P/N=N
Scan Rate (V/s)=(variable)
Segment=5
Sample Interval (V)=0.001
Quiet Time (sec)=2
Sensitivity (A/V)=1e-7

For cyclic voltammetry with metal-binding peptides, copper solution and individual peptide solution were mixed together at appropriate concentration prior to measurements. Select copper individual peptide solutions were mixed together right before the voltammetry measurement.

### Metal-Binding Peptide Absorbance Scan:

Peptide stock solutions were prepared by resuspension in water to final concentration of 10 mM. Metal CuCl2×2H2O and NiCl2×6H2O stock solutions were prepared by resuspension in water to final concentration of 10 mM. Metal and peptide solution were mixed in transparent 96-well plates with buffer in either water or SC/Glc media at final concentrations: 1 mM metal ion, 1 mM peptide, 1×SC/Glc media, 50 mM Tris/HCl (pH 7.5) and 0.1 mM NaClO4. Absorbance scan was measured on Infinite 200 Pro plate reader (Tecan) from 250 nm to 900 nm wavelength in triplicate wells.

### Yeast Culture pH Measurement:

Yeast culture in triplicate was grown overnight in YPD media. Next day, cultures were washed twice with sterile water. 6-mL cultures with yeast ODi 0.15 (as measured in 96-well plate at 200-μL volume) were prepared in SC/Glc media alone or with 50 mM Tris-HCl buffer and/or 0.1 M NaClO4 as final concentrations. Cultures were incubated with shaking and pH was measured in the culture supernatant (cells were spun down for 2 min at 3,300 rpm) at different time points using standard pH glass electrode. After each measurement, culture pellets were resuspended by vortexing before returning for further incubation.

Natural Peptides with Bisulfide Bonds:

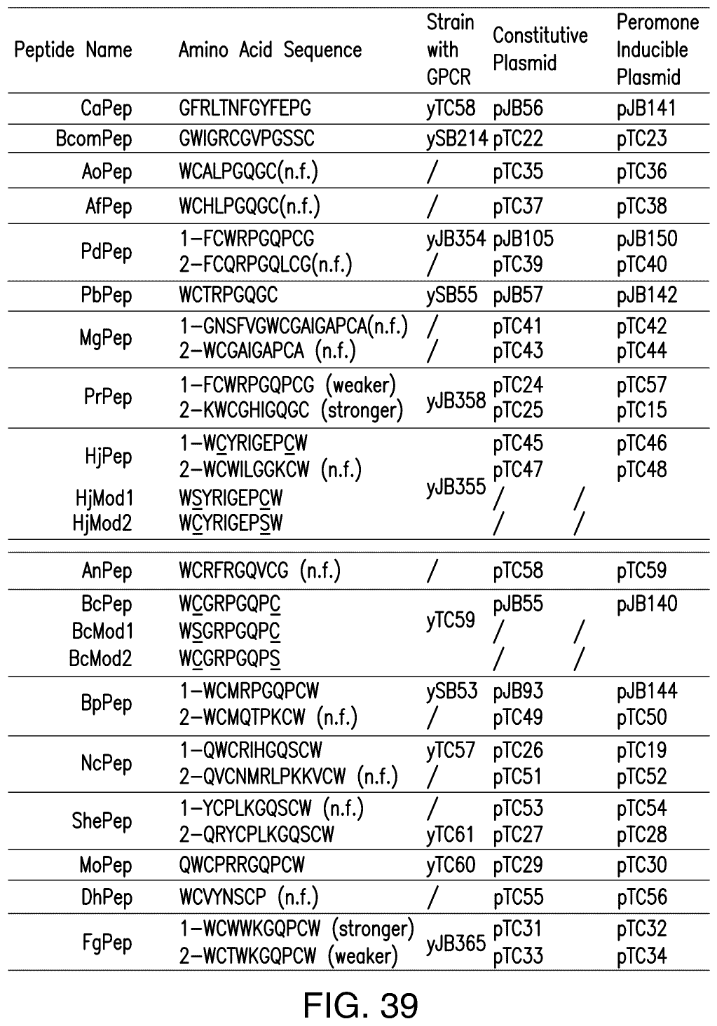

First, fungal pheromone peptides which contain two cysteine residues (C) in their sequence and had also been validated for orthogonal GPCR activation were identified. 24 peptides were reported and peptide CaPep was selected as a negative control which does not have any cysteines (FIG. 39). The peptides have different sequences and each of them have two cysteines placed at different positions within the sequence in order to validate if neighboring amino acids and the distance between two cysteines affect redox potential. Peptides of the same name which have small differences in their amino acid sequence (PrPep and FgPep) activate their cognate GPCR, but to a relatively different extent of fluorescence intensity (stronger or weaker). Based on peptides' ability to activate their putative cognate fungal GPCRs, which was determined by Billerbeck et al., some peptides are deemed non-functional. However, they were still taken into consideration as a possible communication channel from living cell secreting those peptides and electrode sensing them, as they are able to form disulfide bond. Therefore, in order to have a library of peptide secretion plasmids, which can be easily transformed into yeast to actively secrete them, peptides which were already cloned into plasmids were identified and 40 missing ones were cloned.

Additionally, yeast strains that have plasmids with constitutively expressed GPCRs which are responsive to their cognate functional peptides were identified and four missing ones were transformed. Previously cloned plasmids and transformed yeast strains were created and are labeled as pJB or yJB, respectively. Additional, transformed yeast strains are labeled as ySB. Newly cloned plasmids and transformed yeast strains are labeled as pTC and yTC, respectively (FIG. 39). Plasmids with two different modes of expression were made. Plasmids with constitutive peptide expression continuously secrete the peptide in transformed yeast, while plasmids with inducible peptide expression have a promoter pFUS1 which drives peptide expression upon GPCR activation.

Second, ten different synthetic functional peptides were treated with dimethyl sulfoxide (DMSO) to fully oxidize them, or with dithiothreitol (DTT) to fully reduce them. To oxidize, stock peptide solution was serially diluted in water in order to prepare different peptide concentrations for dose response validation. Then, 100% DMSO was added to each peptide dilution at final concentration of 12.2%. Peptides were incubated on benchtop for 1.5 h and then added to cells with final DMSO concentration at 1.22%. To reduce, stock peptide solution was serially diluted in water in order to prepare different peptide concentrations for dose response validation. Then, 30 mM DTT was added to each peptide dilution at final concentration of 3.65 mM. Peptides were incubated on benchtop for 1.5 h and then added to cells with final DTT concentration at 0.365 mM.

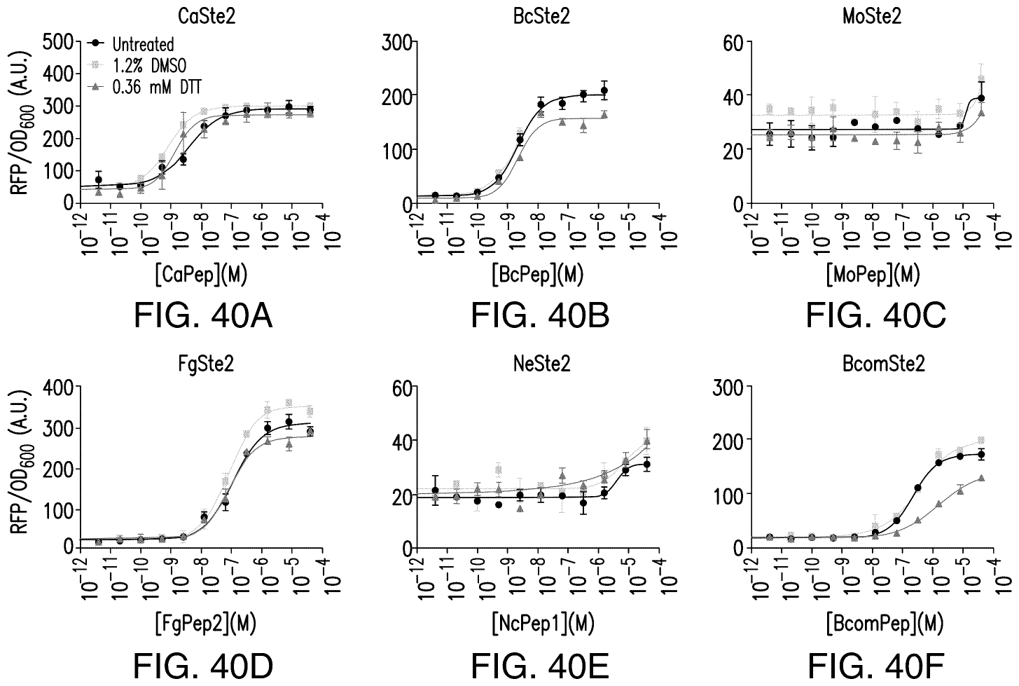

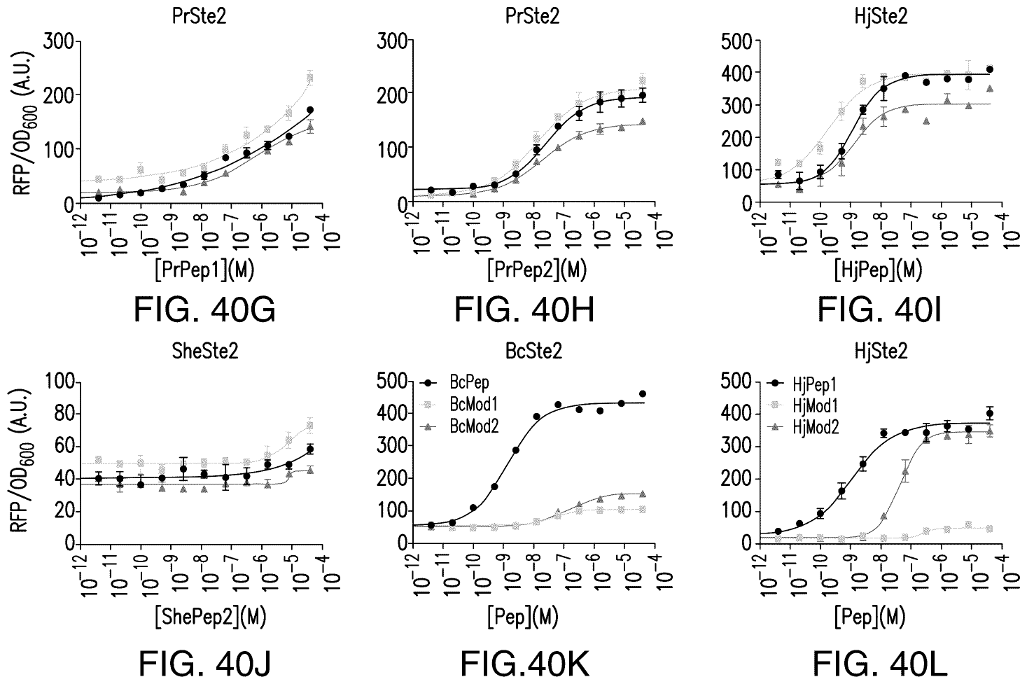

After the treatment, the peptides were added to responsive yeast strains which express appropriate cognate GPCRs and give fluorescent readout as a response. The dose-response curves of yeast responses with untreated, oxidized and reduced cognate peptides were compared (FIG. 40).

### Metal-Chelating Peptides:

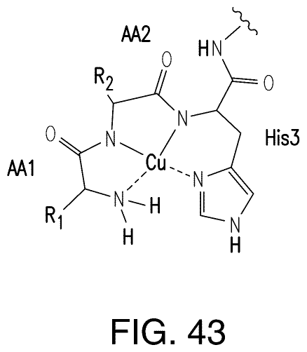

First, to prove the possibility of a mixture of metal-binding peptides rather than individual peptides with different sequences and that their unique redox potentials can be simultaneously captured on the voltammogram, copper and two peptides reported by Mena et al. (His-His-Trp and His-Ala-His), as well as the mixture of the two peptides with copper using cyclic voltammetry (FIG. 42) were tested. The peptides contain ATCUN site which form a copper (II) complex depicted in FIG. 43 that significantly alter metal's electronic and redox properties. Those alterations proved to be peptide sequence specific and the complex formed by His-His-Trp peptide with Cu2+ gave a unique peak at 0.8 V and Cu2+-His-Ala-His complex formed a peak at 1.1 V. When those two complexes were mixed together, their peaks were still resolvable within 0.1 V range.

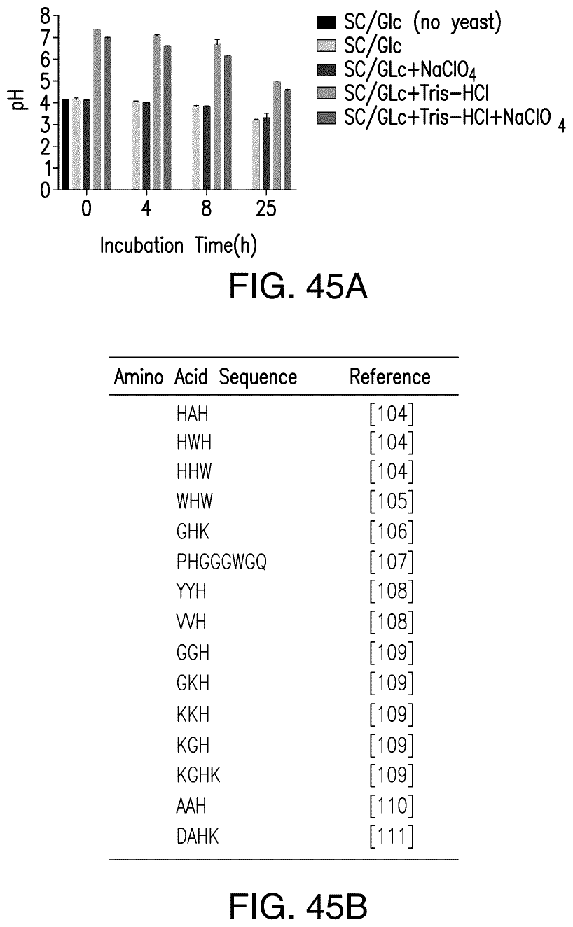

In order to have a large library of metal-binding peptides which can be used in parallel, a literature search of other reported peptides was performed (FIG. 45B). 13 additional peptides which were investigated previously for their copper- and nickel-binding properties were investigated. The majority of peptides contain canonical ATCUN site (FIG. 43) with first two N-terminal random amino acids and histidine as the third amino acid in the sequence. There were three reported peptides which do not have ATCUN site (HHW, WHW and PHGGGWGQ), however, they contain at least one histidine and one tryptophan residue.

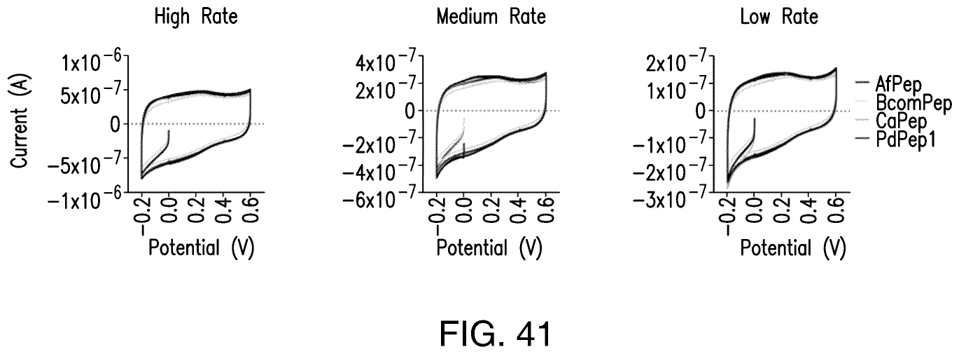

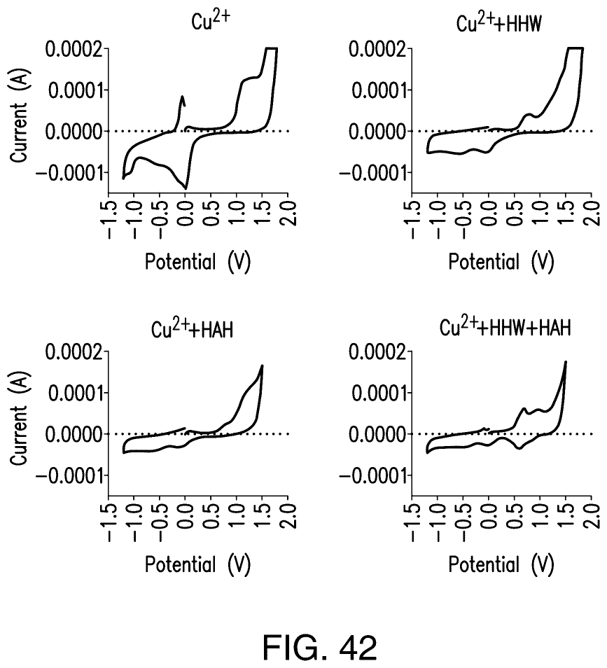

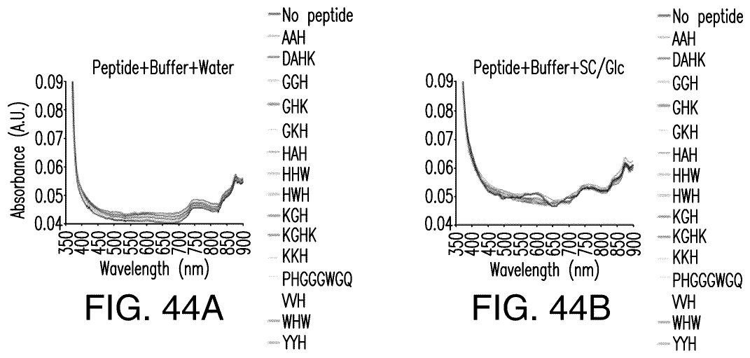

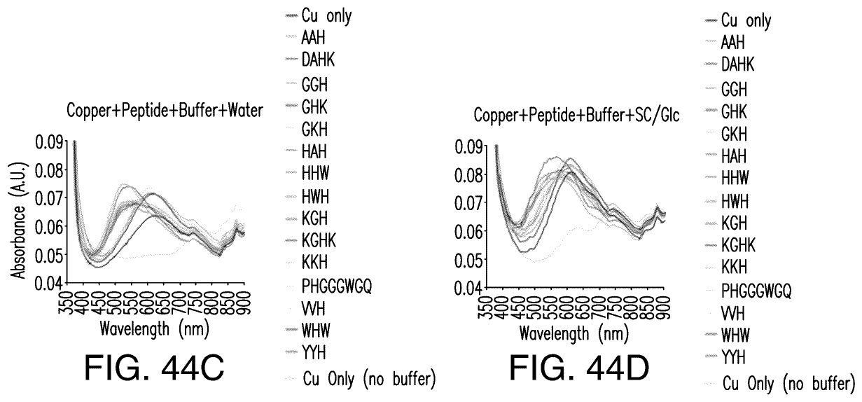

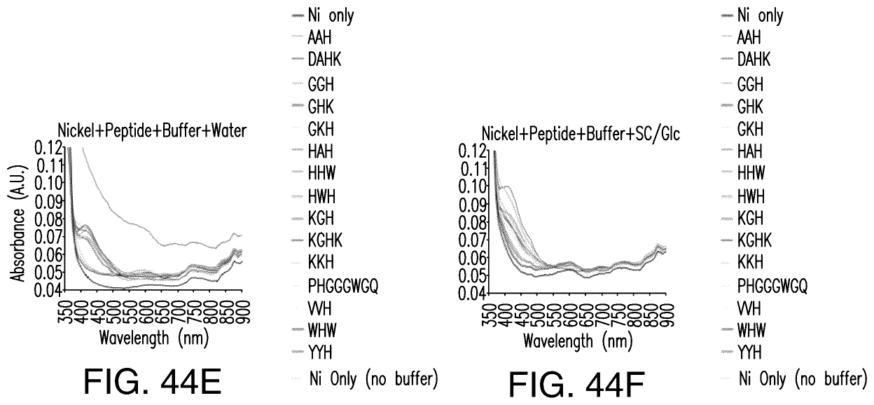

Next, to investigate the effect of voltammetry measurement and cell culturing conditions on the absorbance of metal-peptide complexes, their absorbance values in buffered electrolyte solution used previously for cyclic voltammetry measurements (FIG. 41 and Mena et al.) and standard yeast culturing SC/Glc media (FIG. 44) were compared.

### Results

Natural Peptides with Bisulfide Bonds:

None of the tested peptides showed complete inactivity in either oxidized or reduced state, including the negative control (CaPep), which was evident from the dose-response curves showing similar activation spans and EC₅₀ (concentration of peptide required for half-maximal activation) between differently treated peptides. To further investigate if the presence of disulfide bridges within peptides affect their binding to GPCRs, point mutations in BcPep and HjPep1 (BcMod1, BcMod2, HjMod1 and HjMod2 in FIG. 39) were introduced. Each of the two cysteines within the peptides were changed into serines (S) in order to prevent the formation of disulfide bridge. Serine was selected as a substitute, since it is structurally most similar to cysteine. The rationale was that if the peptide binds to its cognate receptor in reduced form (disulfide bridge not formed), then peptides with point mutations would bind as strongly to the receptor as the original peptide, since they cannot form disulfide bridge. If the peptides with point mutations have diminished binding and activation of their cognate receptor, then the peptide's active form may contain the disulfide bridge, but such difference in activation can also have peptide sequence-specific origin. Dose-response curves of modified peptides compared to the original peptides show weaker response when point mutations were introduced (FIG. 40). For BcPep point mutations, substitution of either cysteines led to 4-fold decrease in maximal activation compared to the original BcPep. Regarding HjPep1 point mutations, substitution of the N-terminal cysteine (HjMod1) led to milder response attenuation with equally strong maximal activation as the original HjPep1, but significantly shifted EC₅₀ to higher concentration by one order of magnitude. Substitution of C-terminal cysteine (HjMod2) practically completely diminished the response. Such vast difference in response of the two modified peptides implies sequence-specific origin for different binding affinities to the receptor, rather than oxidation state.

Lastly, in order to determine if different natural peptides with two cysteine residues could be differentiated by an electrode in a communication setting where a living cell secretes the peptide as redox mediator, whether those peptides can be distinguished using cyclic voltammetry was validated (FIG. 41). Peptides used for cyclic voltammetry measurements were diluted to appropriate concentration in appropriate electrolyte solution. Measurements were done using CHI 760D potentiostat (CH Instruments, Inc., Austin, TX) and glassy carbon electrode with following settings: Init E (V)=0; High E (V)=0.6; Low E (V)=−0.2; Final E (V)=0; Init P/N=N; Scan Rate (V/s)=(variable); Segment=5; Sample Interval (V)=0.001; Quiet Time (sec)=2; Sensitivity (A/V)=1e-7. For cyclic voltammetry with metal-binding peptides, copper solution and individual peptide solution were mixed together at appropriate concentration prior to measurements. Select copper individual peptide solutions were mixed together right before the voltammetry measurement. Three peptides from FIG. 39 (AfPep, BcomPep and PdPep1), as well as CaPep as a negative control were selected and their voltammograms measured. None of the tested peptides showed any unique and distinguishable potential peaks.

### Metal-Chelating Peptides:

SC/Glc media generally increased background absorbance by approximately 0.1 A.U. compared to water sample. Peptides alone did not show prominent absorbance peaks in 400-800 nm range. Copper-peptide complexes showed resolvable absorbance peaks in 500-700 nm range in both water and SC/Glc media. Copper alone also had an absorbance peak at 630 nm, however, that peak is diminished in copper solution without buffer. Nickel-peptide complexes show less prominent peaks compared to copper-peptide complexes in 400-450 nm range. Additionally, some copper-peptide complexes have absorbance maxima at potentially resolvable wavelengths. E.g., copper complexes with AAH and DAHK have absorbance maxima at 520 nm and copper complexes with PHGGGWGQ, KKH, GHK and HHW have absorbance maxima at 620 nm, so potentially they could be distinguished in a mixture by measuring absorbance at their designated peaks. Significant absorption interference could be expected from the non-chelated copper which shows a prominent peak at 630 nm. The source of this interference is copper complexation with Tris-HCl buffer which also strongly chelates copper ions.

The buffering effect and buffer selection in yeast culturing conditions and voltammetry electrolyte solutions were further investigated. For effective binding of metal-chelating peptides and metal ions, pH of the solution plays a critical role. At low pH, the amino groups within ATCUN site are protonated, which prevents them to form complex with the metal ion. For that reason, buffering yeast culturing media is an important component to maintain stable pH at which metal-peptide complexes can form. In order to investigate how effectively Tris-HCl buffer maintains the pH of yeast culture, a fresh culture of lab strain yeast with 50 mM Tris-HCl buffer, as well as 0.1 M NaClO4, were prepared since a supporting electrolyte has to be used for voltammetry measurements of the formed metal-peptide complexes (FIG. 45A). SC/Glc media per se has pH≈4 and as expected, NaClO4 electrolyte solution did not change that. As yeast culture is incubated over time, the pH remains relatively the same and it most drastically drops after one day of incubation to ≈3.5. Yeast culture buffered by Tris-HCl (pH 7.4) has starting pH 7.4. Parallel addition of NaClO4 reduced the buffering capacity and the pH dropped to 7. The buffer manages to maintain the culture pH up to eight hours of incubation (pH≈7 with buffer alone and ≈6.5 with buffer and electrolyte) and it significantly drops to pH 4.5-5 after one-day incubation. For future directions, in order to effectively maintain cell culture pH in the presence of supporting electrolyte solution, higher buffer concentration and/or higher buffered pH may be considered. Since Tris-HCl is not optimal buffer for neither copper-peptide complex measurements due to copper chelation (FIG. 44), nor for long-term buffering of yeast cultures (FIG. 45A and Prins & Billerbeck), alternative buffers may be considered. Based on previous study of copper complexation by commonly used buffers, MOPS (3-(N-morpholino)propanesulfonic acid) with pKa 7.2, CHES (N-cyclohexyl-2-aminoethanesulfonic acid) with pKa 9.3, or CAPS (N-cyclohexyl-3-aminopropanesulfonic acid) with pKa 10.4 could be used as buffers which do not complexate copper and can maintain neutral to basic pH.

### Peptides Containing Unnatural Amino Acids:

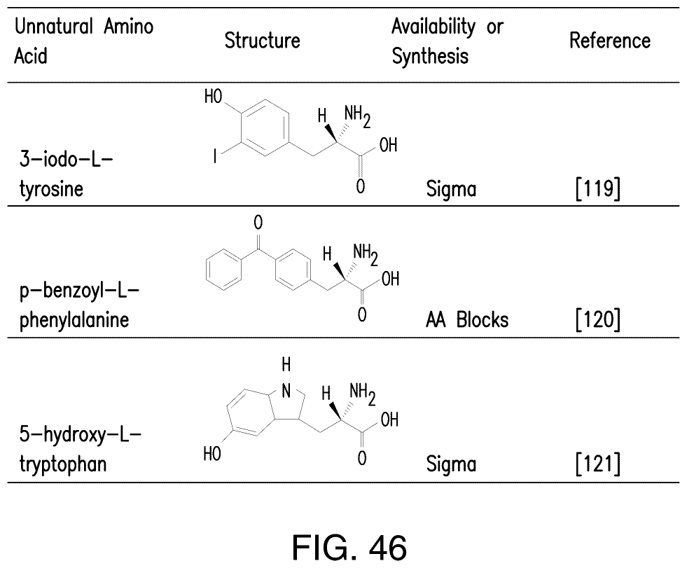

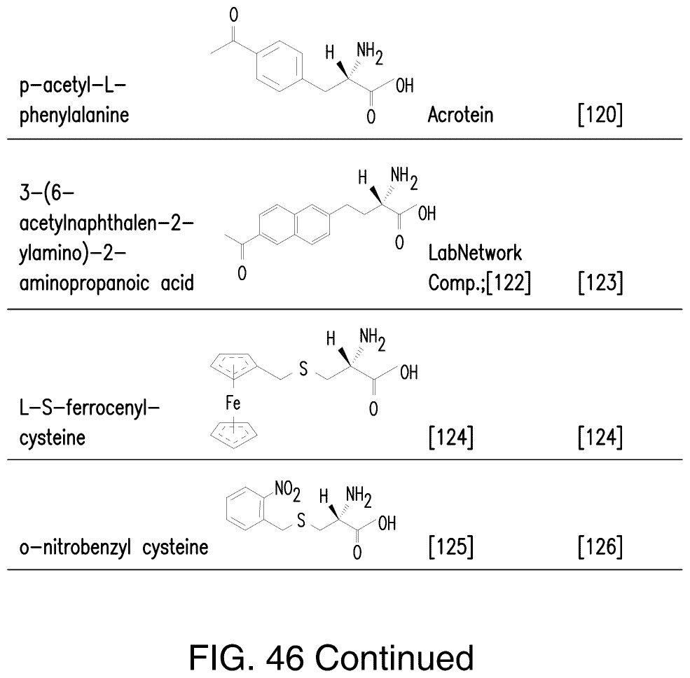

Lastly, in order to diversify the redox potentials of the secreted peptides by living cells and consequently to diversify the signals which the electrical devices could recognize, yeast can be engineered to genetically incorporate redox-active unnatural amino acids (RA-UAAs). Redox active peptides containing tyrosine and tryptophan analogs can be synthesized along with carbonyl-, metal-containing and metal-binding unnatural amino acid residues. It was demonstrated that tyrosine and tryptophan residues in proteins can be oxidized at carbon electrodes and peptides containing these residues can be detected down to picomolar concentrations using constant current chronopotentiometric stripping analysis. Also, the presence of other residues adjacent to tyrosine or tryptophan, such as arginine or lysine, can affect the oxidation peak. From the literature, seven RA-UAAs were selected based on their previously reported successful genetic incorporation into eukaryotes (FIG. 46). Commercially unavailable RA-UAAs can be synthesized following the protocols from the literature. The sequences of developed amino acid-tRNA synthetase open reading frames (ORFs) can be used and incorporation of the corresponding RA-UAAs by using tandem mass-spectrometry of the cell culture supernatants of engineered yeast constitutively expressing the unnatural peptides can be evaluated.

In order to further increase the efficiency of UAA incorporation in yeast the observations from Chin et al. can be followed. The efficient UAA incorporation in yeast was described by making nonsense-mediated mRNA decay pathway deficient strain (upf1Δ) and by introducing a promoter containing the consensus A- and B-box sequences upstream of the E. coli tRNA to drive transcription which is then cleaved post-translationally to yield the mature tRNA. The upf1 ORF can be deleted using an established CRISPR/Cas9 system for S. cerevisiae.

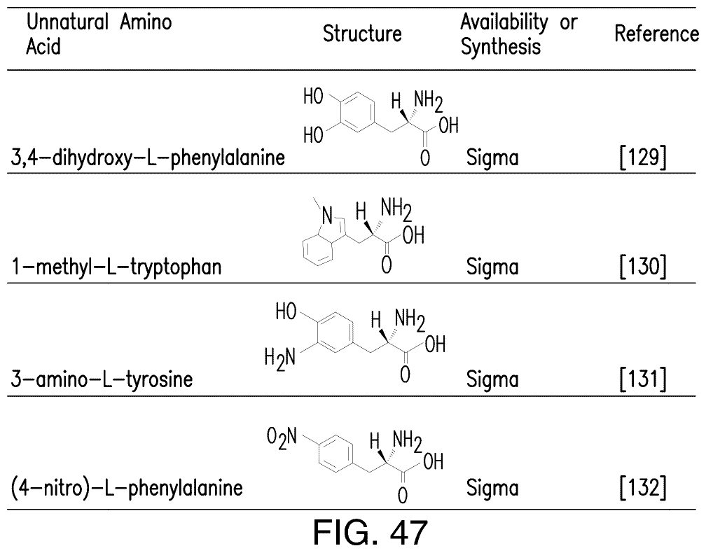

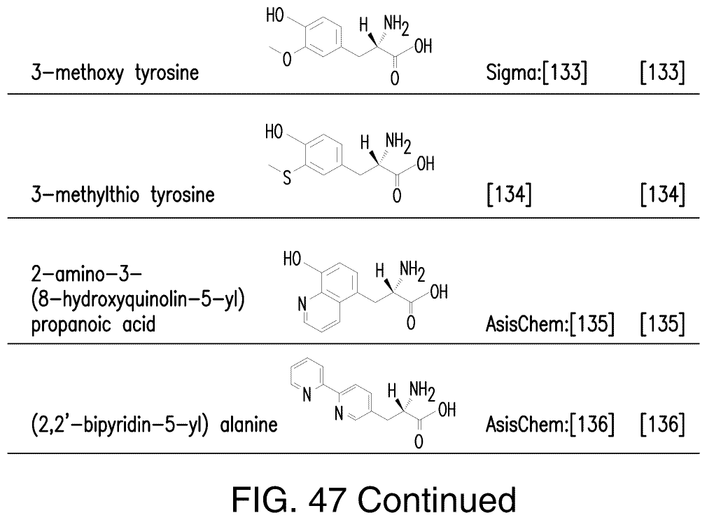

Finally, if the desired redox diversity is not reached with the peptides developed above, eight additional RA-UAA (FIG. 47) can be incorporated and tested. In order to incorporate this suite, orthogonal E. coli UAA-tRNA synthetase-tRNA pairs have to be designed, which are orthogonal to the yeast protein biosynthetic machinery, using a de novo selection protocol.

### DISCUSSION

This example validated natural peptides capable of making intramolecular disulfide bridges and natural metal-binding peptides, as well as the buffering efficiency of buffer/electrolyte solution in standard yeast culture, since pH plays an important role in metal-peptide complex stability. In addition, to further expand the library of unique redox-active peptides for multichannel communication, an experimental workflow for incorporation of redox-active unnatural amino acids into peptides that can be secreted by yeast was incorporated.

Initially tested repertoire of natural fungal pheromone peptides which contain two cysteine residues that were reported by Billerbeck et al. did not prove to be suitable as modular redox mediators for two reasons. First, the disulfide bond oxidation state in these peptides did not significantly affect their ability to activate their cognate GPCRs (FIG. 39), which means it would not be possible to have peptide oxidation/reduction by the electrode as a mean to activate peptide responsive living yeast cell. Second, these peptides do not have distinguishable redox potential peaks on cyclic voltammograms (FIG. 40), which would prevent parallel recognition of those peptides being secreted by the living yeast cell. For those reasons, other more suitable types of redox-active peptides were investigated.

The next promising type of redox-active peptides that could serve as modular redox mediators that were investigated are metal-binding peptides. Unlike disulfide bonds, metal ions are strongly electroactive and most importantly, their redox potential can be modulated by complexation with ligands. Nitrogens in peptide backbone, histidine's imidazole ring and tryptophan's indole ring can chelate copper and nickel ions which serve as a foundation for establishing the metal ion as a direct common redox mediator whose redox potential can be modulated by the selection of chelating peptide sequence. As peptides of different metal-binding sequences are secreted from a living cell, supplemented metal ion in the media then forms different complexes which can be distinguished by the electrode. The findings of Mena et al. where they determined that different amino acid side chains within metal-binding ATCUN site modulated chelated copper's redox potential were expanded. Two copper-binding peptides (HHW and HAH) with copper and measured their voltammograms (FIG. 42) were investigated. The copper redox potential peaks in different peptide complexes could be distinguished which encouraged the expansion of the metal-binding peptide library with more peptides of different sequences. Through literature search of previously reported and validated metal-binding peptides, the investigation expanded to a total of 15 peptides. All of the copper complexes showed absorbance peaks in 500-700 nm range, including copper alone. Since copper did not show absorbance in aqueous solution without the buffer, the copper absorbance peak is actually of copper-buffer complex, since it is known that Tris/HCl buffer can chelate copper, since it contains hydroxy groups. For that reason, in order to prevent background absorbance of free copper, an alternative buffer should be used (e.g., MOPS, CHES, or CAPS). Regarding nickel complexes, peptides GHK, HHW and PHGGGWGQ did not show absorbance peaks, while other nickel-peptide complexes showed absorbance peaks in 400-450 nm range in aqueous solution. However, some of those complexes have diminished peaks in SC/Glc media. Based on these results, either copper or nickel could be used as central metal ions, depending which peptides are wanted to be measured. Within copper-peptide complexes, some complexes have absorbance peaks at different wavelengths and they could potentially be distinguished in a mixture by measuring absorbance at their designated wavelengths.

Finally, utilization of unnatural amino acids as redox-active moieties within peptides were investigated in order to further expand the library of redox-active peptides which can be modularly used for living yeast communication with electronics. In vivo genetic incorporation of unnatural amino acids is a well established field in synthetic biology. The technology is based on engineering of orthogonal aminoacyl-tRNA synthetase/tRNA pairs used with nonsense, rare, or 4 bp-codons to incorporate non-canonical amino acids into proteins in various organisms, both prokaryotic and eukaryotic. Herein, a workflow of redox-active unnatural amino acid incorporation into living yeast was established. For initial incorporation, redox-active amino acids that were already incorporated into eukaryotes (FIG. 46) were searched, as it would be straightforward to translate into yeast already developed orthogonal aminoacyl-tRNA synthetase/tRNA pairs which are derived from bacteria. The unnatural amino acids were selected based on the presence of oxidizing/reducing or metal-binding groups. If distinguishable redox potential diversity is not reached, the unnatural amino acid incorporation can be expanded with de novo design of orthogonal aminoacyl-tRNA synthetase/tRNA pairs (FIG. 47). Another important factor should be considered when selecting amino acids is their commercial availability. Incorporation engineering priority can be given to commercially available amino acids, compared to those which need in-lab organic synthesis, for the sake of simplicity. The last variable that has to be considered when using unnatural amino acids within peptides as redox mediators is that those amino acids have to be supplemented to the yeast culture in order for yeast to uptake them and incorporate into biosynthesized peptides. The presence of free redox-active amino acids in the media can also interfere with the redox potential measurements of secreted redox-active peptides containing those same amino acids.

This example made progress towards establishing redox-active peptides as modular mediators between living organisms and electronics. Metal-binding peptides showed the greatest promise as redox mediators secretable by yeast, since metal ions are strongly electroactive and their redox potential can be modulated by natural peptide sequence without the need of extensive engineering of unnatural amino acid incorporation in yeast. Once established, this bioelectronic interface could significantly advance the field of cell-based diagnostics by enabling facile potentiostatic activation or sensing of multiple cellular signals simultaneously on solid-state electronic devices.

### REFERENCES

1. C. Tea. Expanding Biosensing Capabilities of Engineered Yeast. Columbia University 2022, 13-32.
2. S. Billerbeck et al., “A scalable peptide-GPCR language for engineering multicellular communication,” Nature Communications, vol. 9, no. 1, p. 5057, 2018.
3. S. Mena et al., “Drastic Effect of the Peptide Sequence on the Copper-Binding Properties of Tripeptides and the Electrochemical Behaviour of Their Copper(II) Complexes,” Chemistry—A European Journal, vol. 24, no. 20, pp. 5153-5162, 2018.
4. R. T. Pflaun and W. W. Brandt, “Metal-Amine Coördination Compounds. I. Copper(II) Complexes,” Journal of the American Chemical Society, vol. 76, no. 24, pp. 6215-6219, 1954
5. R. C. Prins and S. Billerbeck, “A buffered media system for yeast batch culture growth,” BMC Microbiology, vol. 21, no. 1, pp. 1-9, 2021.
6. H. E. Mash, Y. P. Chin, L. Sigg, R. Hari, and H. Xue, “Complexation of copper by zwitterionic aminosulfonic (good) buffers,” Analytical Chemistry, vol. 75, no. 3, pp. 671-677, 2003.
7. J. L. Dearling, J. S. Lewis, D. W. McCarthy, M. J. Welch, and P. J. Blower, “Redox-active metal complexes for imaging hypoxic tissues: Structure-activity relationships in copper(II) bis(thiosemicarbazone) complexes,” Chemical Communications, vol. 0, no. 22, pp. 2531-2532, 1998
8. T. S. Young and P. G. Schultz, “Beyond the Canonical 20 Amino Acids: Expanding the Genetic Lexicon,” The Journal of Biological Chemistry, vol. 285, no. 15, pp. 11039-11044, 2010.
9. X. Caia, R Gustav, P. A. M. Farias, H. Shiraishi, J. Wang, and E. Palecek, “Potentiometric stripping analysis of bioactive peptides at carbon electrodes down to subnanomolar concentrations,” Analytica Chimica Acta, vol. 332, pp. 49-57, 1996.
10. J. A. Reynaud, B. Malfoy, and A. Bere, “The electrochemical oxidation of three proteins: RNAase A, bovine serum albumin and concanavalin A at solid electrodes,” Journal of Electroanalytical Chemistry, vol. 116, no. C, pp. 595-606, 1980.
11. V. Brabec and V. Mornstein, “Electrochemical behaviour of proteins at graphite electrodes. I. Electrooxidation of proteins as a new probe of protein structure and reactions,” Biochimica et biophysica acta, vol. 625, no. 1, pp. 43-50, 1980.
12. V. Dorcák, V. Ostatná, and E. Pale\check{ }cek, “Electrochemical reduction and oxidation signals \check{ } of angiotensin peptides. Role of individual amino acid residues,” Electrochemistry Communications, vol. 31, pp. 80-83, 2013.
13. J. W. Chin, T. A. Cropp, J. C. Anderson, M. Mukherji, Z. Zhang, and P. G. Schultz, “An Expanded Eukaryotic Genetic Code,” Science. vol. 301, pp. 964-967, 2001.
14. T. A. Cropp and J. W. Chin, “Reprogramming the amino-acid substrate specificity of orthogonal aminoacyl-tma synthetases to expand the genetic code of eukaryotic cells,” Nature Protocols, vol. 2, no. 10, pp. 2590-2600, 20.

The contents of all figures and all references, patents and published patent applications and Accession numbers cited throughout this application are expressly incorporated herein by reference.

1. A genetically-engineered sensor cell for detecting the presence of a fungal species or a virus in a sample, comprising:
(a) at least one heterologous G-protein coupled receptor (GPCR) that binds to an analyte derived from the fungal species or the virus, wherein the analyte is a ligand for the heterologous GPCR; and
(b) a reporter gene, wherein binding of the analyte to the heterologous GPCR triggers the expression of the reporter gene and indicates the presence of the fungal species in the sample,
wherein the fungal species is selected from the group consisting of A. nidulans, A. fumigatus, A. terreus, A. flavus, A. niger, A. clavatus, A. oryzae, A. novofumigatus, A. lentulus, A. viridimutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marnefei, P. carinii, P. murina, P. wakefieldiae, C. dubliniensis, C. auris, C. pseudohaemulonii, C. haemuloni, C. duobushaemulonis, C. metapsilosis, C. orthopsilosis and a combination thereof and/or wherein the virus is a respiratory virus, a hemorrhagic virus, a gastrointestinal virus, an exanthematous virus, a hepatitis virus, a sexually transmitted virus and/or a neurological virus.

2. (canceled)

3. The sensor cell of claim 1, wherein the analyte is a peptide analyte.

4. The sensor cell of claim 1, wherein the peptide analyte is an analyte disclosed in Table 2.

5. The sensor cell of claim 1, wherein the sensor cell is S. cerevisiae.

6. The sensor cell of claim 1, wherein the heterologous GPCR is a Ste2 receptor from the fungal species and/or wherein the heterologous GPCR comprises an amino acid sequence that is at least about 80%, at least about 85%, at least about 90%, at least about 95%, at least about 98% or at least about 99% homologous to any one of the GPCR amino acid sequences disclosed in Tables 5, 6 or 8 or a GPCR engineered by directed evolution to bind the analyte.

7. (canceled)

8. (canceled)

9. The sensor cell of claim 1, wherein the reporter gene encodes a fluorescent protein, a visible light pigment, a redox peptide and/or a metal-chelating peptide.

10. (canceled)

11. A kit for detecting the presence of a fungal species or a virus and/or for detecting a healthcare-associated infection in a sample comprising one or more sensor cells of claim 1.

12. (canceled)

13. The kit of claim 11, wherein:
(a) the one or more sensor cells are provided in a test tube;
(b) the kit further comprises means for obtaining the sample from a subject; and/or
(c) the kit further comprises a protease.

14. (canceled)

15. (canceled)

16. A method of detecting the presence of a fungal species or a virus in a sample, comprising:
(a) contacting the sample with a sensor cell comprising at least one heterologous G-protein coupled receptor (GPCR) that binds to an analyte derived from the fungal species, wherein the analyte is a ligand for the heterologous GPCR;
(b) binding of the analyte present in the sample to the heterologous GPCR, wherein binding of the analyte to the heterologous GPCR triggers an appearance of a reporter; and
(c) detecting the appearance of the reporter, wherein the appearance of the reporter indicates the presence of the fungal species or the virus in the sample,
wherein the fungal species is selected from the group consisting of A. nidulans, A. fumigatus, A. terreus, A. flavus, A. niger, A. clavatus, A. oryzae, A. novofumigatus, A. lentulus, A. viridinutans, A. udagawae, N. fischeri, T. citrinoviride, T. arundinaceum, T. longibrahiatum, T. harzianum, T. guizhouense, T. lentifore, T. virens, T. asperellum, T. gamsii, T. atroviride, B. dermatitidis, B. silverae, C. immitis, T. marnefei, P. carinii, P. murina, P. wakefieldiae, C. dubliniensis, C. auris, C. pseudohaemulonii, C. haemuloni, C. duobushaemulonis, C. metapsilosis, C. orthopsilosis and a combination thereof and/or wherein the virus is selected from the group consisting of a respiratory virus, a hemorrhagic virus, a gastrointestinal virus, an exanthematous virus, a hepatitis virus, a neurological virus, a sexually transmitted virus and a combination thereof.

17-27. (canceled)

28. The sensor cell of claim 1, wherein the respiratory virus is selected from the group consisting of an influenza, a respiratory syncytial virus, a parainfluenza virus, a metapneumovirus, a rhinovirus, a coronavirus, an adenovirus, a bocavirus and a combination thereof.

29. (canceled)

30. The sensor cell of claim 28, wherein the coronavirus is SARS-CoV-2, MERS-CoV, SARS-CoV or a variant thereof.

31. (canceled)

32. (canceled)

33. The sensor cell of claim 30, wherein the variant of SARS-CoV-2 is selected from the group consisting of a SARS-CoV-2 alpha variant, a SARS-CoV-2 beta variant, a SARS-CoV-2 delta variant, a SARS-CoV-2 gamma variant, a SARS-CoV-2 epsilon variant, a SARS-CoV-2 kappa variant, a SARS-CoV-2 iota variant, a SARS-CoV-2 eta variant, a SARS-CoV-2 lambda variant, a SARS-CoV-2 mu variant, A SARS-CoV-2 omicron variant or a SARS-CoV-2 zeta variant.

34. The sensor cell of claim 28, wherein the analyte is derived from a nucleocapsid protein and/or a spike (S) protein of the coronavirus.

35. (canceled)

36. The sensor cell of claim 1, wherein the hemorrhagic virus is an ebolavirus.

37. The sensor cell of claim 36, wherein the ebolavirus is selected from the group consisting of Zaire ebolavirus, Sudan ebolavirus, Tai Forest ebolavirus, Bundibugyo ebolavirus, Reston ebolavirus, Bombali ebolavirus and a combination thereof.

38. (canceled)

39. The sensor cell of claim 36, wherein the analyte is derived from a small secreted glycoprotein of the ebolavirus.

40. The sensor cell of claim 1, wherein the analyte is a protein of the virus or a peptide analyte derived from a protein of the virus.

41. (canceled)

42. The sensor cell of claim 41, wherein the analyte comprises an amino acid sequence that is at least about 80%, at least about 85%, at least about 90%, at least about 95%, at least about 98% or at least about 99% homologous to any one of the sequences shown in Table 3.

43-71. (canceled)

72. A genetically-engineered sensor cell for detecting the presence of a protein variant in a sample, comprising:
(i) (a) at least one heterologous G-protein coupled receptor (GPCR) that binds to a wild type protein and one or more variants of the protein;
(b) a protease that cleaves one or more of the protein variants; and
(c) a reporter gene, wherein binding of the wild type protein to the heterologous GPCR triggers the expression of the reporter gene; or
(ii) (a) at least one heterologous G-protein coupled receptor (GPCR) that binds to a wild type protein and one or more variants of the protein:
(b) a protease that cleaves the wild type protein; and
(c) a reporter gene, wherein binding of the one or more protein variants to the heterologous GPCR triggers the expression of the reporter gene.

73. (canceled)

74. (canceled)

75. The sensor cell of claim 72, wherein the protein variant is a variant of a protein from a virus and/or wherein the protein variant comprises an amino acid sequence that is at least about 80%, at least about 85%, at least about 90%, at least about 95%, at least about 98% or at least about 99% homologous to any one of the sequences shown in Table 3.

76. The sensor cell of claim 75, wherein the virus is SARS-CoV-2 or a variant thereof.

77-82. (canceled)

83. The sensor cell of claim 72, wherein the heterologous GPCR is a GPCR comprising an amino acid sequence that is at least about 80%, at least about 85%, at least about 90%, at least about 95%, at least about 98% or at least about 99% homologous to any one of the sequences shown in Table 6 or 8 or a GPCR engineered by directed evolution to bind the protein variant.

84-101. (canceled)

102. A genetically-engineered sensor cell for detecting an analyte in a sample, comprising:
(a) a heterologous G-protein coupled receptor (GPCR) that binds to an analyte derived from the fungal species, wherein the analyte is a ligand for the heterologous GPCR; and
(b) a reporter gene, wherein binding of the analyte to the heterologous GPCR triggers the expression of the reporter and indicates the presence of the fungal species in the sample, wherein the genetically-engineered sensor cell comprises a mutation and/or deletion in one or more of the following endogenous genes: Mfa1, Mfa2, Mf(alpha)1, Mf(alpha)2, Bar1, Far1, Sst2, Gpr1, Gpa2, Ste2, sfGFP, LEU2 and URA3.

103-112. (canceled)

113. A method of detecting the presence of an analyte in a sample, comprising:
(a) contacting the sample with a sensor cell of claim 102;
(b) binding of the analyte present in the sample to the heterologous GPCR, wherein binding of the analyte to the heterologous GPCR triggers an appearance of a reporter; and
(c) detecting the appearance of the reporter, wherein the appearance of the reporter indicates the presence of the analyte in the sample.

114. (canceled)


SEQUENCE LISTING (392 sequences)

SEQ ID NO: 1 - protein, 9 aa, unidentified
  REGION 1..9: note = A. nidulans
WCRFAGRIC

SEQ ID NO: 2 - protein, 9 aa, unidentified
  REGION 1..9: note = A. nidulans
WCRFRGQVC

SEQ ID NO: 3 - protein, 9 aa, unidentified
  REGION 1..9: note = A. fumigatus
WCHLPGQGC

SEQ ID NO: 4 - protein, 9 aa, unidentified
  REGION 1..9: note = A. terreus
WCWLPGQGC

SEQ ID NO: 5 - protein, 9 aa, unidentified
  REGION 1..9: note = A. flavus
WCSLPAQGC

SEQ ID NO: 6 - protein, 9 aa, unidentified
  REGION 1..9: note = A. niger
WCVLPGQPC

SEQ ID NO: 7 - protein, 9 aa, unidentified
  REGION 1..9: note = A. clavatus
WCELPGQGC

SEQ ID NO: 8 - protein, 9 aa, unidentified
  REGION 1..9: note = A. oryzae
WCALPGQGC

SEQ ID NO: 9 - protein, 377 aa, unidentified
  REGION 1..377: note = A. nidulans
MASNFDPWNQTITFRLEDGTPFDISVDYLDGILQYSIRACVNYAAQLGASVILFVILVLLTRAEKRASCLFWLNSLALLLNFARLLCDVLFFTGNFVRIYTLISADESRVTASDLATSIVGAIMTALLLTTIEISLVLQVQVVCSNLRRIYRRALLCVSAVVATATIAIRYSLLAVNIRAILEFSDPTTYNWLESLATVALTISICYFCVIFVTKLGFAIRLRRKLGLSELGPMKVVFIMGCQTLVIPVIVSITHYVSDVPELQTNVLTIVALSLPLSSIWAGTTIDKPVTHSNVRNLWQILSFSGYRPKQSTYIATTTTATTNAKQCTHCYSESRLLTEKESGRNNDTSSKSSSQYGIAVEHDISVRSARRESFDV

SEQ ID NO: 10 - protein, 373 aa, unidentified
  REGION 1..373: note = A. fumigatus
MGSTFDPFSQSITLRGPAENEITIPIETVDFFYQYCIRICINYGAQLGASIVLFIILLLLTRPEKRRSSVFVLNITALLLNVSRLICQLIYFTSEFVRLYAYFGNDFSRVPPSAYADSILGVVLVTLLIVCIEASLVLQAQVVCANLRRGYRRILLAVSIVMALVPIIFRFVYMVQNCWHIMHAAETIRLNWLESATNVVITISICFFSAVFITKLGFAMHLRKRLGMTDFGPMKVIFVMGCQTMAIPAIFSILQYIVSVPELASNIFSLVTISLPLSSIWAGFTIDQRARDSGMSSSRNNLFQALSFSKEAKTRQSSTVTYASQPSSKSHTLCYAELSPAKQQSDAEAAYGIAVERDISIHSCRKDGKAPNV

SEQ ID NO: 11 - protein, 373 aa, unidentified; identical to SEQ ID NO: 10
  REGION 1..373: note = A. terreus

SEQ ID NO: 12 - protein, 374 aa, unidentified
  REGION 1..374: note = A. flavus
MTMAIMDSKFDPYSQNLTFHAADGTPFQVPVMTLNDFYQYCIQICINYGAQFGASVIIFIILLLLTRPDKRASSVFFLNGGALLLNMGRLLCHMIYFTTDFVKAYQYFSSDYSRAPTSAYANSILGVVLTTLLLVCIETSLVLQVQVVCANLRRRYRTVLLCVSILVALIPVGLRLGYMVENCKTIVQTDTPLSLVWLESATNIVITISICFFCSIFIIKLGFAIHQRRRLGVRDFGPMKVIFVMGCQTLTVPALLSILQYAVSVPELNSNIMTLVTISLPLSSIWAGVSLTRSSSTENSPSRGALWNRLTDSTGTRSNQTSSTDTAVAMTYPSNKSSTVCYADQSSVKRQYDPEQGHGISVEHDVSVHSCQRL

SEQ ID NO: 13 - protein, 378 aa, unidentified
  REGION 1..378: note = A. niger
MASTDPFAQNVTFHTGDGTPFEVNVQEIDTFLQYCIQICTNYGAQLGASIVLFVILLLLTRPEKRSSSVFILNCAALLSNVGRLFCQILYFTTDFVRVYAYFSGDFSRVPTSAYANSVIAVVFYTILVICIEASLVLQVQVICANLGRVYRQVLLSLSIVVALIPVGFRMAMMVENAIAIMAAESSIPLIWLQSAENITATISICFFCAVFVAKLGFAIRQRRRLGVRDFGPMKVIFIMGCQTLVIPATFSIIHYIAAVPALSSNVATLVTISLPLSSIWAGVALTKVSSGAGTGSGTGSGSSGRNLWNVLSFPVWDSRNTTSTTTSSSTPTVNPVSTCYADNGSSKQCKQQTNTEEIGITVEHEISVQSFKREKDMV

SEQ ID NO: 14 - protein, 370 aa, unidentified
  REGION 1..370: note = A. clavatus
MNSTFDPFEQSFTLQRSDGKTFNTSLVIADHNMEYMLRLSINYGTQLGASIVLLLILMLLTRPEKRLSSVFLLNVAALASNIVRLVCQISYFSSGFAKTYALLAHDFSRVAQATYASQVMASVFFTLVLVCVEISLVLQVQVVCSNLRRRYRHLLLGASILVTLIPIGFRLAFMVENCIVIVRSGNMKNYHWLESATNIVATVSICFFCAIFVVKLGFAIKMRRRLGVKDFGPMKVIFIMGCQTMIIPALFSICQYWVNIPEFSHNLLTLVTISLPLSSIWAGFTLDSSNNTANSTASRRNLWNVLSSNGTVQDKPNLSACSPVASPNTTCYSDPYSGKPHHQDLEAGFGISVEHDISIHSYRKDAHSDV

SEQ ID NO: 15 - protein, 369 aa, unidentified
  REGION 1..369: note = N. fischeri
MNSTFDPWTQNITLTQSDGTTVISSLALADDYLHYMIRLGINYGAQLGACAVLLLVLLLLTRPEKRVSSVFVLNVAALLANIIRLGCQLSYFSTGFARMYALLAGDFSRVSRGAYAGQVMASVFFTIVFICVEASLVLQVQVVCSNLRRQYRILLLGASTLAALVPIGVRLTYSVLNCMVIMHAGTMDHLDWLESATNIVTTVSICFFCAVFVVKLGLAIKMRKRLGVKQFGPMRVIFIMGCQTMTIPAIFAICQYFSRIPEFSHNVLTLVIISLPLSSIWAGFALVQANSTARSTESRHHLWNILSSDGATRDKPSQCVSSPMTSPTTTCYSEQSTSKPQQDPENGFGISVAHDISIHSFRKDAHGDI

SEQ ID NO: 16 - protein, 374 aa, unidentified; identical to SEQ ID NO: 12
  REGION 1..374: note = A. oryzae

SEQ ID NO: 17 - protein, 378 aa, unidentified
  REGION 1..378: note = A. novofumigatus
MNSTFDPWTQNITLTQSDGTTVISSLALADDYLHYMIRLSINYGAQLGACAVLLLVLLLLTRPEKRISSVFVLNVTALLANIIRLGCQLSYFSTGFARMYALLAGDFSRVSRGAYAGQVMASVSFTIVLICVEASLVLQVQVVCSNLRRRYRILLLGASTLAALVPIGVRLTYSVLNCMVIMHAGTMDHLDWLESATNIVTTVSICFFCAVFVIKLGLAIKMRKRLGVKQFGPMRVIFIMGCQTMTIPAIFAICQYFSRIPEFSHNVLTLVIISLPLSSIWAGFALDQANSTARSTESRHHLWNILSSDGATRDKPTQCVSSPMTSPNTTCYSEQSTSKPQQDPENGFGISVAHDISIHSFRKGANGIFEQSLHLRNA

SEQ ID NO: 18 - protein, 369 aa, unidentified
  REGION 1..369: note = A. lentulus
MNSTFDPWTQNITLTQSDGTTVISSLALADDYLHYMIRLSINYGAQLGACAVLLLVLLLLTRPEKRVSSVFVLNVAALLANIIRLGCQLSYFSTGFARMYALLAGDFSRVSRGAYAGQVMASVFFTIVLICVEASLVLQVQVVCSNLRRQYRILLLGASTLAALVPIGVRLTYSVLNCMVIMHAGTMDHLDWLESATNIVTTVSICFFCAVFVVKLGLAIKMRKRLGVKQFGPMRVIFIMGCQTMTIPAIFAICQYFSKIPEFSHNVLTLVIISLPLSSIWAGFALDQANSTARSSESRHHLWNILSSDGATRDKPSQCVSSPMTSPNTTCYSEQSTSKPQQDPENEFGISVAHDISIHSFRKDAHGHL

SEQ ID NO: 19 - protein, 369 aa, unidentified
  REGION 1..369: note = A. viridinutans
MNSAFDPWTQNITLTRSDGTTVISSLALADEFLRYMIRLSINYGAQLGACVVLLLVLLLLTRPEKRVSSVFVLNVAALLANIIRLGCQLSYFTTGFARIYALLAGDFSRVSRGAYAGQVMASVFFTLVLICVEVSLVLQVQVVCSNLRRQYRILLLGASTLAALVPIAVRLTYSVQNCMVIMHAGTMDHLDWLESATNIVTTVSVCFFCAVFVVKLGLAIKLRKRLGVKQFGPMRVIFIMGCQTMTIPAIFAICQYFSEIPEFSHNVLTLVIISLPLSSIWAGFTLEQANSTARSTESRHNLWNILSSDGAVRDKPGQCISSPLTSPNTTCYSQHSTGKPQQDPENGFGIAVAHDISIHSYQKDARGDV

SEQ ID NO: 20 - protein, 369 aa, unidentified
  REGION 1..369: note = A. udagawae
MNSTFDPWTQNITLTRSDGTTVISSLALADEFLRYMIRLSINYGAQLGASVVLLLVLMLLTRPEKRVSSVFMLNVAALLANIIRLGCQLSYFSTGFAKMYALLAGDFSRVSRGAYAGQVMASVFFTLVLICVEVSLVLQVQVVCSNLRRQYRILLLTASTFAALVPIAVRLTYSVQNCMVIMHAGTMDHLDWLESATNIVTTVSICFFCAVFVVKLGLAIKMRKRLGVKQFGPMRVIFIMGCQTMTIPAIFAICQYFSKIPEFSHNVLTLVIISLPLSSIWAGFALEQANPTARSTESRHNLWNVLSSDGAARDKPSQCISSSLTSPNATCYSEQSTSKSEQDPANRFGISVAHDISIRSYQKDAHGDV

SEQ ID NO: 21 - protein, 396 aa, unidentified
  REGION 1..396: note = T. citrinoviride
MAFDPYTQNITILLSPSAPPISIPIPLIDAFNDETASIITNYAAQLGAAITMLLVLVAATPSSRLLRASTLLHAAALLVCVARTVLLAYFFLTPFSHFYQVWTGDFSAVPAWNYRASIAGTVLSTLLAVVTDAALVHQAWTMVSLFAPRIKFAVCALSLLITLLAVSFRVAYTVIQCEGIADLAAPRRYAWLIRTTLICNICSIGWFCALFNSKLISHLVSNRGVLPSRRAMTPMEVLIMANGILMIVPVIFAILEWHHFINFESGSLTPTSIAIILPLASLAAQRIATSSASKHHNHPSSSSARYKASRDSASRTPLNAKPGSLFTTTTCNSTASSSAAQRTRSVDASGMLAAAAGPARGGDFIDPIDLELRQIDGYAQPNEVHFQQDEGQQFHK

SEQ ID NO: 22 - protein, 393 aa, unidentified
  REGION 1..393: note = T. arundinaceum
MSFDPYSQNITILTPTPSLVPVPIPIPLIDSFNDETVSIVINYGAQLGASITMLLVVLFTTPSSKFLRASNLLHILALLVCIVRTVLLVFYFLSPFSHFYQVWSGDYSQIPAWNYRTSVAGTVFSMLLTIITDAALMNQAWTMVSLWPSKTKYILSFSSMLVTLLAVSFRVAYTVIQCEGIYDLIAPRRFAWLIRTTLIFNICSICWFCALFNSKLIIHLITNRGVLPSRRAMNPMEVLIMANGILMIVPVVFAVLEWHHFINFEAGSLTPTSVAIILPLGSLAAQRMAQSAQDASSNISSYPSSSGAAYHGRYKNTRDSGSRTPLKAGSLFSTTTNSSAAAARSQLTAENTTLSPVQDIIDPIDLELRQIDGYLQPKETHVKAESSESRFHK

SEQ ID NO: 23 - protein, 297 aa, unidentified
  REGION 1..297: note = T. longibrahiatum
FNPYTQNITILLSPSAPPISIPIPLIDAFNDETVSIITNYAAQLGAALAMLLVLLAATPPSRLLRPGTPLVHALALLVCVVRTVLLIYFFLTPFSHFYQVWTGDFSQIPAWNYRASIAGTVLSTLLTIVTDAALVNQAWTMVSLFAPRTKRIVCLFSVLVALLAISFRVAYTVIQCESIAALAAPRRFAWLIRATLLLNIGSIGWFCALFNSKLVSHLVTNRGVLPSRRAMSPMEVLIMANGILMIVPVIFAILEWHHFINFESGSLTPTSIAIILPLSSLAAQRIATSPSSSSSSS

SEQ ID NO: 24 - protein, 385 aa, unidentified
  REGION 1..385: note = T. harzianum
MAFNPYTQNITIIASPDHPPILIPITLIDTFNDETVSIVINYGAQLGASLTMLLVILLTTPSAKFLRASCLLHVLALIVCAVRTVFLAFYFLSPFSHFYQVWSGDYSQIPAWNYRTSIAGTILSMFLATIAEGALMYQAWTMVSLFANKAKYAICFLSLLIILITTAFRVAFTVVQCESILNLETSRKYAWLFKTALIFNTCSMAWFCALFNSKIVHHLISNRGVLPSRRAMTPMEVLIMANGILMFIPFIFAILEWCHFINFEAGSLTPTSIAIVLPLASIAAQRISQAPSTPSSEPSYPSSSYQAQYKSTRDSSRTPLKSGSLFSSITNTSSATRSHVGDTTVLAPVRDVIDPIDLELRQIDGYLQPKEIHSRAEAGESQLHK

SEQ ID NO: 25 - protein, 385 aa, unidentified
  REGION 1..385: note = T. guizhouense
MAFNPYTQNITVIVSPDYPPILIPIPLIDTFNDETVSIVINYGAQLGASLAMLLVILLTTPSSKFLRASCLLHVLALIVCAVRTVFLAFYFLSPFSHFYQVWAGDYSEIPAWNYRTSIAGTILSMFLATIAEGALMYQAWTMVSLFANKAKYAICFLSLLIILITTGFRVAFTVIQCESILSLETPQKYAWLFKTALIFNTCSMAWFCALFNSKIVHHLISNRGVLPSRRAMTPMEVLIMANGILMFIPFIFAILEWCHFINFEAGSLTPTSIAIVLPLASIAAQRISQAPSTPSSEPSYPSSSYQAQYKSTRDSSRTPLKSGSLFSTISKTSSATRSHVADTTVLAPVRDVIDPIDLELREIDGYSQPKEIHSRAEAGESQLHK

SEQ ID NO: 26 - protein, 385 aa, unidentified
  REGION 1..385: note = T. lentifore
MAFNPYTQNITIIVSPDYPPILIPIPLIDAFNDETVSIVINYGAQLGASLTMLLVILLTTPSSKFLRASCLLHVLALIVCAVRTVFLAFYFLSPFSHFYQVWSGDYSQIPEWNYRTSIAGTILSMFLAVIAEGALMHQAWTMVSLFANKAKYTICFLSLLIILITTAFRVAFTVVQCESILSLESSRKYAWLFKTALIFNTCSMAWFCALFNSKIVHHLISNRGVLPSRRAMTPMEVLIMANGILMFIPFFFAILEWCHFINFEAGSLTPTSIAIVLPLASIAAQRISQAPSTPSSEPSYPSSSYQAQYKSTRDSSRTPLKSGSLFSAITNTSSATRSHIADTTVLAPVRDVIDPIDLELRQIDGYVQPKEIQSRTEASESQLHK

SEQ ID NO: 27 - protein, 386 aa, unidentified
  REGION 1..386: note = T. virens
AKMAFDPYTQSITIHSPPGPPIKIPIPEIDRFNEETASIIINYGAQLGASITMLLVILLTVPTQKFLRASNLLHILALLVCVFRTVFLVLYFISPFSHFYQVWSGDYSQIPAWNFRTSIAGAVLPMLLTVTTEAALMHQAWTMVSLFANKTKYAITFLSLLLTLIAIAFRVAYTVAECKSIIDLVSAEKYAWLIKTTLIWNTCSVGWFCALFNSKLVWHLVSNRGVLPSRRAMTPMEVLIMANGILMIFPVIFATLEWYHFINFEAGSLTPTTVAIILPLGTLVAQRLSQTSPAPSTDPSSYPSSSYQARYKATRDSSRTPFKSGSLFSATTNSSAPRSQLVDTTVLAPVRDVIDPIDLELRQIDGYLQPKEIHVRAEPGEGQFHK

SEQ ID NO: 28 - protein, 388 aa, unidentified
  REGION 1..388: note = T. asperellum
  VARIANT 22: note = Xaa can be any naturally occurring amino acid
MAFDPYTQNITIAITPNVSIPXPIPVIDIFNDETVNIVLNYGTQLGAAITMLLVVLFTTPPAKAFRTANLLHVVGLLVCIVRTVLLALYFNSPFAHFYAVWTNDYSQVPAWTFRESVAGTVFSMLLVIISDVSLINQAWTMVSLWPSRTKYALCFVSLLISLLSIAARTAFTVIQCEGIYELDPPMQFAWLVHAMVILNICTTLWFCALFNSKLVIHLITNRGVLPSRRAMSPMEVLVVTNGVLMIIPVIFAFIEFRPVVNFESSSLTPASIALILPLGSLVAQRLAQTNQNASPNLFSYPSSSGAGGYKNNHSSGSTTPLKPGSLFATSTNSSISAARSKSNSQTITAVPTRDIVDPIELELRQIDGYLQPQETHVSAELGGSRSHK

SEQ ID NO: 29 - protein, 389 aa, unidentified
  REGION 1..389: note = T. gamsii
MSFNPYAQNVTIRISPALPALHIPLHFIDSFNDETVSIVLNYGTQLGAAIMMLLVVLFTTPPSKFLRTAHLLHLLGLLVCIVRTVLLALYFESPFAHFYAVWSGDYSGVPSWTYRESVAGTVLSMLLVVVNDMALMNQAWTMVSLWPTATKFVLCAVSLLFALLSVAARTAFTVIQCKAIYALEPAVQFAWLVHAMVIINICTTLWFCALFNSKIIIHLIANRGVLPSRRAMSPMEVLVVTNGALMIIPVIFAFIEFRPILNFESSSLTPASITLILPLGSLVAQRLAYTNQNAVPSLSGYPSSTGAGGYKNNRSSGSNTPLKAGSLFATSTNNSISAARSHSTSRNTTTVCARDVVDPIELELRQIDGYLQPQETHISADLGGSQSHK

SEQ ID NO: 30 - protein, 392 aa, unidentified
  REGION 1..392: note = T. atroviride
MALFDPSTQDVTIHISPSAPPLHIPLRAIDGFNDETVSIVLNYGTQLGAAITMLLVLLFTTPPSKFLRTAHLLHLLGLLVCIVRTVLLALYFESPFAHFYAVWSNDYSAVPAWTYRESVAGTVFSMLLVVVNDVALMNQAWTMVSLWPTGTKLVLCAVSLLVSLLSVAARTAFTVIQCKAIYDLTPAVQFAWLVHAMVILNICTTVWFCALFNSKIIIHLITNRGVLPSRRAMSPMEVLVVTNGALMIIPVIFAFIEFRPILNFESSSLTPASITLILPLGSLVAQRLAQTNQNAIPSLSGYPSSTGAGGYKNNRGSGSGSHTPLKAGSLFATSTNNSVSAARSHSTSRNTTTVPARDIVDPIELELRQIDGYLQPQETHISAELGGSQSHK

SEQ ID NO: 31 - protein, 355 aa, unidentified
  REGION 1..355: note = B. dermatitidis
MASSFDPFDQNVVFHQADMTPFNVSIHDLDDFVQYGTRVCINYASQLGASVIAIIMLALLTQSEKRRLPVFSLNISALIMNFARLLCMTIYFTTGFNNPYAFFSGDYSRVPGSAYADSILGIVFTTILVICMETSLVIQTQVVCATLPEIQRYLLLGVSLVIALLAIGFRMGLMVENCIAIMNASNFAPFIWLQSASNITITMSTCFFSAIFVAKLGYALVTRRRLGMTRFGAMQVMFIMSCQTMVIPAIFSIIQYPFPGYEMNSNLLTLVAVFLPLSSLWAAAATKSSFETLNSGPHQYLWSSERSNSTSSATRHPGSFSQNQATLRSGGSVATTLSPDQLDRLYTDLDVSVKA

SEQ ID NO: 32 - protein, 355 aa, unidentified
  REGION 1..355: note = B. silverae
MASSFDPFDQNVVFHQADGTPFNVSIHDLDEFVQYGTRVCINYAAQLGASVIAIIMLALLTRSEKRRLPVFSLNLAALIMNFARLLCMTIYFTTGFNNPYAFFSGDYSRVPGSAYADSILGIVFTTILVICMETSLVIQTQVVCATLSEIQRYLLLAVSLVIALLAIGFRMGLMVENCIAIINASNFGPFIWLQSASNITITISTCFFSAIFVVKLGYALVTRRRLGMTRFGAMQVMFIMSCQTMVIPAIFSIIQYPFPLYEMNSNLLTLVAIFLPLSSLWAAAATKSSFETLNSGPHQYLWSSERSNSTSSAPGQHGNFSQNQSTLRSGGSVATTLSPDQLDRLYTDLDTSAKA

SEQ ID NO: 33 - protein, 357 aa, unidentified
  REGION 1..357: note = T. marneffei
MSFNPYEQNITLTASDGSAMDIPLPYFDEYVQEGVVSCINYGCQLGATIVIFVLLCLLTRSKKRSSIVFCLNITALLLNMFRLICQVLLYTGAWFETYAYFTGDISRVTRGNFADSILVSVMDSLLQVVMEASLVTQTNVVCTNMSRWQKTAILTVSVGLAFSSIGFRMAQMGLESVLIVENKPFDEFLWLQKLNTILTTTSIIYFSVVLICKLGYTIYLRRQLGVHQFSPMQIIFIMSCQTMIVPAILSILQFSSVNIHHINNDVVTFVVVSLPVTTLWASNVLPGRAAVHDAESGPPLWNKLSTPRLNSVGSDQFRRKPASPSNPIAFPSTMNFQGSELAKSVTIGTRLEGSISP

SEQ ID NO: 34 - protein, 370 aa, unidentified
  REGION 1..370: note = C. immitis
MVDTSGFGNNFDPFTQPVRLYYPNGTDFHRSLAEIDWILRDNTNQSIAYGCQLGASLTMIIILLLLTPPGKRRSPVFMLNAAALVLNFGRILCSCIYYTTGFSDVYAMLAPDFSRVSVGAYANTFLGAILHALLLICVEISLLFQTHVLCSTIRSAYRRLALSFSVVLVLAAIGCRLMQTIENCRATINLKSFASFIWLQSLTNILTTVSICYFSTIFVAKLGFAIYARRTLGLTGFGAMQVMFIMSCQSMIVPARRLTHDLAIFSILHHFIDAAEMAPLVLTLVTLSLPLSSMWAAQAVKNSFEGDNRGPTQQRHLFTQGSSTAQTSMASTLVAKRTSSPDHLDRLYPDIDAIEIRVDRDFTVDIEKGQ

SEQ ID NO: 35 - protein, 531 aa, unidentified
  REGION 1..531: note = C. dubliniensis
MNSTIIPPNDSGDIIINYLIPGLNESIEIPFHLLDSFQTDQTKLALIMGITIGSCSMTLIFLISIMYKTNKLSNIFNNNINIKTIFQWINQMIFNEKINLNKHKQNDSYNNNIIVGKGFYKLFLFYLNSLILLIGIIRSGCYLNYNLGPLNSISFVFTGWYNESISTSKLSFISSDITNGFKCILYGLVEISLGFQVYIMFKTSNLKIWGLMASLLSISLGLIVVALQINLTILSHIRFVKATTTTTTTNNNETSTSIVTGGYVINSIWMDLPTILFSISINIMTILLIGKLIIAIRIRRYLGLKQFDSFHILLIGFSQTLIIPSIILIIHYFYLSKSKDSLLQQISLLLIILMLPLSSLWAQTANNIHNINSSPSLSFISRHHSSDNNSNINTIVNGNNNHLYNSNSNSNSNSNSNGNGNGKFSKLNTNGSSPFTLKGDSGSLMLQMTSNSNNGNNGNINAGNVNAGNNFSIPVSGIDAQLPPDIEKILHDEDNINNYKLLNINNENINDGDIIINDEGMITKQITIKRV

SEQ ID NO: 36 - protein, 376 aa, unidentified
  REGION 1..376: note = C. auris
MEFTGDIVLKYTLGGEEYSSTFEQLDSSVNRSLELGVVHGIAIACGVLLMVLAWVIIIKKKNPIFVLNQLTLLLMVIKSSLYLAFLFGPLSSLTYKFTRVLPHDKWHAFHVYIATNVIHTLLIATVEMTLVFQIYIIFKSPEVRHLGYILTGAASALALTIVALYIHSTVISAVQLKEQLLMHEIKITNSWVNNVPIILFSASLNVVCIILIAKLALAIKTRRYLGLKQFDGLHILMITSTQTFIVPSVLMIVNYKQSSSYSTLLANISVILVVCNLPLSSLWAASANNSSTPTSSANTVFSRWDSKFSDTETIAHELPLIPGKAEKLQLVSPITEKGDTHTMCESHGDQDSIDKMLDDIEGAVMTTEFNLNNRTV

SEQ ID NO: 37 - protein, 376 aa, unidentified
  REGION 1..376: note = C. pseudohaemulonii
MDFSNDIVLNYTVAAEEYQLTFGILDSTVDRTLQLCVVHGMAIGCGALLMLLSWVVIIKKTTPMFILNQLTLMLMIIKSALFLCFLFGPLSSLTYKFTGLFPEERWHAFHIYIATNTIHTLLIATVEATLVFQIYVVFKSPEVRNLGYIMTCAAGALATTIVSLYIHSTVISARQLRQQLMEQDVTPTNSWVNNVPVILFSASLNVVCIILISKLALAIKTRRYLGLKQFDGLHILIITSTQTFIVPSVLMIVNYRQSTSYSTLLANISVILAVCNLPFSSLWATSANNSPTPTSSPNTVFSRWDSKISDTETLNQDMPLIPGKAEKIQLVSPTTEKGDVQTVCGSDGDQDLIDRILDDMDGAVMTTELNLRDRMC

SEQ ID NO: 38 - protein, 377 aa, unidentified
  REGION 1..377: note = C. haemuloni
MEFSDNDIVLNYTVGPEIYQLTFGILDNTVDRSLQLCVVHGVAIGCGILLMLLSWVVIIKKRTPMFILNQLTMVLMIIKSALYLCFLFGPLSSLTYKFTGLLPEERWHAFDIYIATNVIHTLLIATVEATLVFQIHVIFKSPEVKHLGYVLTAAAGSLAVTIVSLYIHSTVVTAIQLRNQLMEQDVTPTNSWVNNVPVILFSASLNVVCIILISKLALAIRTRRYLGLKQFDGLHILIITSTQTFIVPSVLMIVNYRQSTSYSTLLANISVILAVCNLPFSSLWATSANNSTHPTSSPNTVFSRWDSKLSDTETLAHEVPLLPGKAEKIQLVSPTTEKGDVQTMCDSNGDQDSIDRMLDDIDGAIMTTELNIRDKLV

SEQ ID NO: 39 - protein, 376 aa, unidentified
  REGION 1..376: note = C. duobushaemulonis
MDFSGDSLLNSTATAEEYQLMFGILDSTVERALQLCVVHGMAIGCGALLMLLSWVVIIKKTTPTFILNQLTLVLMIIKSALYLCFLFGPLSSLTYKFTGLFPEGRWHAFHIYIATNTIHTLLIASVEATLVFQIYVVFKSPEVRKLGYFMACAAGALATTIVSLYIYSTVLSARQLRQQLLEHEFTPTNSWINNVPVILFSASLNVVCIILIAKLALAIKTRRYLGLKQFDGLHILTITTTQTFILPSVLMIVNYKQSSSYSTLLANISVILVVCNLPFSSLWATSANNSPTPTSSPNTVFSRWDSKTSDTETLNQDTPLIPGKAEKIQLVSPTTEKGDVQTVCGSDGDHDSIDRILDDMDGALMTTELNLRDRMC

SEQ ID NO: 40 - protein, 402 aa, unidentified
  REGION 1..402: note = C. metapsilosis
MDQIVSNLSSGDIFVTITIPNEEDGTYEVPFYVIDQYHNSKMENAVILGATIGACSMLLIMLIGILFKNFNKLKKSLLFNLNFAVLLMLIMRSACYINYLMNNLSSISFFFTGIFNGESFMSSDAANAFKVILVALIELSLTYQIYIMFKTPTLKVWGVIASLLAGALGLATIATQIYTTVMSHINFVNGTTGSPSQIVTAWMDMPTILFSVSVNVLSLFLVCKLGLAIRTRRYLGLKQFDAFHILFIMSTQTMIIPSIILFVHYFDQDDSQTTLVNVSLLLVVISLPLSSLWAQTANNVRRIDTSPSMSFISREASNRSGQETLHSGAKISKYNTSNTVNSTPGTLKDDSSFILDRPIEQDKVDTGLPKDLEKFLNNDFNEGDDGMIAREITMLKTQHPRQ

SEQ ID NO: 41 - protein, 403 aa, unidentified
  REGION 1..403: note = C. orthopsilosis
MNQIVSKLNSGDIIVTFTISNEEDGTYEVPFYVLDEYHRSKMQNAIVLGATIGACSILLIMLIGILFKKFQKLKKSLLFNLNFAILLMLIMRSACYINYLMNNLSSIGFFFTGVFDGESFMSSDAANAFKVILVALIETSLTYQICIMFKTPTLKIWGILASFSAGALGLVTIATQIYTTVMSHRNFVNGTTGSPSQITFAWMDMPTILFSVSVNVLSLFLVCKLGLAIRTRRYLGLKQFDAFHILFIMSTQTMIIPSIILFVHYFYRDDSQTTLVNISLLLVVISLPLSSLWAQTANNVRRIDTSPSMSFISREISNRSGNETLHSSAKISKYNTSNTVNSTPGTLKDDSSFILDRPVSEQKMVDTGLPKDLEKFLNNDFNEGDDGMIAREVTMLKTNHTNQ

SEQ ID NO: 42 - protein, 354 aa, unidentified
  REGION 1..354: note = P. carinii
MVFSPVNQTVLLKNSKGDTIPFLLSDFDEFSLSRAQTSMIFSAQCAMSLLLAIVLILTSKREKRKTLLFFLNIGGLITVFIRACLQCAYLSGTWVSYSVQFLGEFELLSQKDFYVSIIASCIPIFIILFIELSLLIQIRVVYATEKKLQIPLTIAFSIIIIVVIIFWILAAVQNSMAVLSQTHFGHSGLWGSPWPYTVARISFAFSIFIGCMVFIYKLLITIYRRHKMGVKEFGPLQIIFIMSCQTLIIPAILILVDFGVKITGFSSLTQALVVMSLPLSSLWASSKVENNKNNAAPTYYKDMKSIGDYSIESTPSSFTKPPYMGFRKPSYFSEYSRSPFYEEYFDDNGSKLIY

SEQ ID NO: 43 - protein, 367 aa, unidentified
  REGION 1..367: note = P. murina
MAFSPTNQTIFLKNSKGETIPFLLSDFDEFSLSRAQTSMIFSAQCAMSLLLALVLMLTSKREKRKTLLFFLNIGGLVTVFIRACLQCAYLSGTWVSYSVQFLGEFELLSQKDFYISIIASCIPIFIILFIELSLLIQIRVVYATDKRLQIPLTIFFSIIIIIVITFWILAAVQNSMAVLSQTHFGHSGVWGAPWPYTVARISFAFSIFIGCIVFIYKLLITIYRRHKMGVKEFGPIQIIFIMSCQTLIIPAILILIDFGVKITGFSSLTQALVVMSLPLSSLWASSKVEKNKNNVTPTYYKDMKSIGDYSIESTPSSFTKPSYIGFRKPSYFSEYSKSPFYDEYFDDNGSKLDILVEKSLNVFPEKK

SEQ ID NO: 44 - protein, 329 aa, unidentified
  REGION 1..329: note = P. wakefieldiae
MGFSPVNQTVLLKNSNGETIPFLLSDFDYFSLSRAQTSMIFSAQCSMSLLLALVLILTSKKEKRKTLLFFLNIGGLITCTISRRVSIVVAKRLLYINCHIIVVYATDKRFQIPLTTFFSMIIIAVLTFWILAAVQNSMAVLSQTHFGHSGLWGSPWPYTAARISFAFSIFIGCIVFIYKLLVTIYRRHKMGVKEFGPLQIIFIMSCQTLIIPVILILIDFGVKITGFSSLTQALVVMSLPLSSLWASSKVENTKNNVRPTYYNDMKSIGDYSIESTPSSLTKPLYMGFRKPSYFSEYSKSPFYDDYFDDNGCKLDILVEKSLNVLPEKK

SEQ ID NO: 45 - DNA, 1107 bp, unidentified
  misc_feature 1..1107: note = A. fumigatus Ste2 nucleotide sequence
  source 1..1107: mol_type = genomic DNA
atgaactcgacatttgatccttggacgcagaatatcactctgactcagtccgatgggacgacggtcgtctcatctctagcactagcagacgactatctgcactacatgattcgtctcggcatcaactatggcgctcagctcggcgcatgcgccgtactcttcctagtcctcttgctgctgacaagaccagagaaacgcgtctcatcggtctttgtcctgaacgtctctgcgttacttgccaacattatacgtctgggatgccagctcagctatttctctaccggattcgctaggatgtatgcgttgctggctggcgacttctccagagtgtcacgcggtgcttatgccggccaagtcatggcatctgtgttcttcactatagtccttatctgtgtggaggcctccttggtgcttcaggtgcaggttgtctgttccaatctccttcgtcagtatcggatcctgctacttggtgcctcgacgctcgctgcccttgttccgatcggggttcgcttgacttactcggtgatgaactgcatggtgatcatgcacgcaggaaccatggaccatctggattggctagagagtgccacgaatatcgtgacaaccgtcagcatttgctttttctgcgccgtcttcgtcgtcaagcttggactcgccatcaaaatgcgaaagagattgggtgtgaaaaaattcggtccgatgagggtcatctttatcatgggttgccaaactatgaccatcccagccgttttcgccatttgccagtacttcagcagaattccagaattctcgcataacgtcctaaccctcgtcatcatatcgctccctctttcctccatttgggctggattcgcccttgaccaggcaaactccacagcgcgctctaccgagtctcgtcaccatctgtggaacatactctcctctgacggcgccacgagagacaagcccagccaatgtgccaactcccccatgacttctccgaacaccacatgctactcagagcagtcaaccagtaaaccccagcaggatccggagaacgggttcggcatttcggtggcgcatgacatctccatccacagcttccaaaagggtggccacggggatatt

SEQ ID NO: 46 - DNA, 1107 bp, unidentified
  misc_feature 1..1107: note = Codon-optimized A. fumigatus Ste2 nucleotide sequence without internal BsaI restriction sites
  source 1..1107: mol_type = genomic DNA
atgaactcaacatttgacccctggacacaaaacattacactaacacaaagtgacgggactacagtcgtcagttcacttgcactggctgatgactacctgcattacatgatcaggctaggaattaactatggggcacagttaggcgcctgcgccgtactattccttgtcctacttttgcttaccaggcccgaaaagagggtatctagtgtgttcgttttgaacgtgagtgcacttttagccaatataataaggctaggttgtcaactttcctacttttccacgggcttcgcgcgtatgtacgcattgttggccggtgacttcagcagagtgagccgtggagcctatgcgggtcaagtaatggcttcagtattttttacaatagttctgatttgcgtcgaagcctctttagtcctgcaagtgcaggtcgtgtgctccaatcttctgcgtcagtataggattcttctattgggcgcttccaccctggcagctctggtgcccataggcgtacgtctaacatacagtgtgatgaattgcatggtgatcatgcacgctggcacaatggaccatctggactggttagagagcgcaacaaatatcgttacgactgtcagtatttgtttcttttgcgcggtgttcgtagtgaagttgggcctggctataaaaatgaggaagagattgggtgtcaagaagtttggaccgatgagagtaatattcatcatgggctgccaaactatgaccatccctgccgtgttcgcaatctgtcagtatttcagtaggattccagaattctctcataacgttttgaccttagtaatcatttccctgcccttatcaagtatatgggccgggttcgctttggatcaagcgaactctacggctcgtagtacagaatctcgtcaccacctgtggaatattctttcatcagatggtgcgacaagagataaaccttcccagtgtgcaaatagtccaatgacaagtccgaatacgacctgttattccgaacagtcaacgtccaagcctcagcaagatcccgagaacgggtttgggattagcgtagctcatgatatcagtatccactccttccaaaaaggggggcacggcgacatt

SEQ ID NO: 47 - DNA, 309 bp, unidentified
  misc_feature 1..309: note = A. fumigatus PpgA ORF AFUA_6G06360
  source 1..309: mol_type = genomic DNA
atgaaactcctctcacttgttcttgcttccttcgctgccgctgctgtccaggcacacattacgccgtggtgccatcttcctggtcagggttgctacatgcttaagcgcgctgctgacgcctcggatgaagttagacgttcagccagtgccgtcgctgaggccgttgcggaagccttcccgcagagtccgtggtgccatcttcccggtcagggttgcgccaaggcgaagagagccgccgaggccgctgaagaggtgaagcgctctgccgatgcctttgctgaagccatggctgcttttgagaaggaatag

SEQ ID NO: 48 - DNA, 495 bp, synthetic construct
  misc_feature 1..495: note = Synthetic: Native MFalpha1 ORF (SGD:S000006108) - prepro-sequence with ScPep
  source 1..495: mol_type = other DNA
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagaggctgaagcttggcattggttgcaactaaaacctggccaaccaatgtacaagagagaagccgaagctgaagcttggcattggctgcaactaaagcctggccaaccaatgtacaaaagagaagccgacgctgaagcttggcattggctgcaactaaagcctggccaaccaatgtacaaaagagaagccgacgctgaagcttggcattggttgcagttaaaacccggccaaccaatgtac

SEQ ID NO: 49 - protein, 9 aa, synthetic construct
  REGION 1..9: note = Synthetic: peptide
  VARIANT 3: note = Xaa can be any naturally occurring amino acid
  VARIANT 6: note = Xaa can be any naturally occurring amino acid
WCXLPXQGC

SEQ ID NO: 50 - protein, 9 aa, synthetic construct
  REGION 1..9: note = Synthetic: peptide
  VARIANT 3: note = Xaa can be any naturally occurring amino acid
WCXLPGQGC

SEQ ID NO: 51 - protein, 17 aa, synthetic construct
  REGION 1..17: note = Synthetic: T-cell epitope/B-cell epitope
KHWPQIAQFAPSASAFF

SEQ ID NO: 52 - protein, 12 aa, unidentified
  REGION 1..12: note = S. stipites Cognate Pep
WHWTSYGVFEPG

SEQ ID NO: 53 - protein, 12 aa, synthetic construct
  REGION 1..12: note = Synthetic: Int-1Prep
WHWPQYGVFAPG

SEQ ID NO: 54 - protein, 12 aa, synthetic construct
  REGION 1..12: note = Synthetic: Int-2 Prep
WHWPQIAVFAPS

SEQ ID NO: 55 - protein, 12 aa, synthetic construct
  REGION 1..12: note = Synthetic: Target Peptide
KHWPQIAQFAPS

SEQ ID NO: 56 - protein, 12 aa, unidentified
  REGION 1..12: note = L. Elongosporous Cognate Pep
WMWTRYGRFSPV

SEQ ID NO: 57 - protein, 18 aa, synthetic construct
  REGION 1..18: note = Synthetic: Loop 3 Epitope
YQAGSTPCNGVEGFNCYF

SEQ ID NO: 58 - protein, 13 aa, unidentified
  REGION 1..13: note = B.compniacensis Pep
GWIGRCGVPGSSC

SEQ ID NO: 59 - protein, 13 aa, synthetic construct
  REGION 1..13: note = Synthetic: Intermediate #1
GWIPRCGVEGSNC

SEQ ID NO: 60 - protein, 13 aa, synthetic construct
  REGION 1..13: note = Synthetic: Intermediate #2
GWIPCNGVEGFNC

SEQ ID NO: 61 - protein, 13 aa, synthetic construct
  REGION 1..13: note = Synthetic: Target Peptide
GSTPCNGVEGFNC

SEQ ID NO: 62 - protein, 9 aa, unidentified
  REGION 1..9: note = B. bassiana peptide
WCMQTPKCW

SEQ ID NO: 63 - protein, 12 aa, synthetic construct
  REGION 1..12: note = Synthetic: Intermediate #1
WCMQTPKCWLQK

SEQ ID NO: 64 - protein, 12 aa, synthetic construct
  REGION 1..12: note = Synthetic: Intermediate #2
WCMKTTKSWLQK

SEQ ID NO: 65 - protein, 9 aa, Zaire ebolavirus
  SITE 1..9: note = Ebola virus Zaire epitope #2
KTTKSWLQK

SEQ ID NO: 66 - protein, 9 aa, synthetic construct
  REGION 1..9: note = Synthetic: Intermediate #1
WCMKIPKCW

SEQ ID NO: 67 - protein, 9 aa, synthetic construct
  REGION 1..9: note = Synthetic: Intermediate #2
WLQKIPKCW

SEQ ID NO: 68 - protein, 9 aa, Zaire ebolavirus
  SITE 1..9: note = Ebola virus Zaire epitope #3
WLQKIPLQW

SEQ ID NO: 69 - protein, 13 aa, synthetic construct
  REGION 1..13: note = Synthetic: peptide
WHALQLKPGQPMY

SEQ ID NO: 70 - protein, 13 aa, synthetic construct
  REGION 1..13: note = Synthetic: peptide
WHWLALKPGQPMY

SEQ ID NO: 71 - protein, 13 aa, synthetic construct
  REGION 1..13: note = Synthetic: CaPep
GFRLTNFGYFEPG

SEQ ID NO: 72 - protein, 13 aa, synthetic construct
  REGION 1..13: note = Synthetic: CaPep1A
AFRLTNFGYFEPG

SEQ ID NO: 73 - protein, 13 aa, synthetic construct
  REGION 1..13: note = Synthetic: CaPep2A
GARLTNFGYFEPG

SEQ ID NO: 74 - protein, 13 aa, synthetic construct
  REGION 1..13: note = Synthetic: CaPep3A
GFALTNFGYFEPG

SEQ ID NO: 75 - protein, 13 aa, synthetic construct
  REGION 1..13: note = Synthetic: CaPep4A
GFRATNFGYFEPG

SEQ ID NO: 76 - protein, 13 aa, synthetic construct
  REGION 1..13: note = Synthetic: CaPep5A
GFRLANFGYFEPG

SEQ ID NO: 77 - protein, 13 aa, synthetic construct
  REGION 1..13: note = Synthetic: CaPep6A
GFRLTAFGYFEPG

SEQ ID NO: 78 - protein, 13 aa, synthetic construct
  REGION 1..13: note = Synthetic: CaPep7A
GFRLTNAGYFEPG

SEQ ID NO: 79 - protein, 13 aa, synthetic construct
  REGION 1..13: note = Synthetic: CaPep8A
GFRLTNFAYFEPG

SEQ ID NO: 80 - protein, 13 aa, synthetic construct
  REGION 1..13: note = Synthetic: CaPep9A
GFRLTNFGAFEPG

SEQ ID NO: 81 - protein, 13 aa, synthetic construct
  REGION 1..13: note = Synthetic: CaPep10A
GFRLTNFGYAEPG

SEQ ID NO: 82 - protein, 13 aa, synthetic construct
  REGION 1..13: note = Synthetic: CaPep11A
GFRLTNFGYFAPG

SEQ ID NO: 83 - protein, 13 aa, synthetic construct
  REGION 1..13: note = Synthetic: CaPep12A
GFRLTNFGYFEAG

SEQ ID NO: 84 - protein, 13 aa, synthetic construct
  REGION 1..13: note = Synthetic: CaPep13A
GFRLTNFGYFEPA

SEQ ID NO: 85 - protein, 13 aa, synthetic construct
  REGION 1..13: note = Synthetic: CaPep9A10A
GFRLTNFGAAEPG

SEQ ID NO: 86 - protein, 13 aa, synthetic construct
  REGION 1..13: note = Synthetic: CaPep12A13A
GFRLTNFGYFEAA

SEQ ID NO: 87 - protein, 23 aa, synthetic construct
  REGION 1..23: note = Synthetic: SpPep
TYADFLRAYQSWNTFVNPDRPNL

SEQ ID NO: 88 - protein, 23 aa, synthetic construct
  REGION 1..23: note = Synthetic: SoPep
TYEDFLRVYKNWWSFQNPDRPDL

SEQ ID NO: 89 - protein, 23 aa, synthetic construct
  REGION 1..23: note = Synthetic: SjPep
VSDRVKQMLSHWWNFRNPDTANL

SEQ ID NO: 90 - protein, 13 aa, synthetic construct
  REGION 1..13: note = Synthetic: CaPep
GFRLTNFGYFEPG

SEQ ID NO: 91 - protein, 13 aa, synthetic construct
  REGION 1..13: note = Synthetic: BcomPep
GWIGRCGVPGSSC

SEQ ID NO: 92 - protein, 9 aa, synthetic construct
  REGION 1..9: note = Synthetic: AoPep
WCALPGQGC

SEQ ID NO: 93 - protein, 9 aa, synthetic construct
  REGION 1..9: note = Synthetic: AfPep
WCHLPGQGC

SEQ ID NO: 94 - protein, 10 aa, synthetic construct
  REGION 1..10: note = Synthetic: PdPep
FCWRPGQPCG

SEQ ID NO: 95 - protein, 10 aa, synthetic construct
  REGION 1..10: note = Synthetic: PdPep
FCQRPGQLCG

SEQ ID NO: 96 - protein, 9 aa, synthetic construct
  REGION 1..9: note = Synthetic: PbPep
WCTRPGQGC

SEQ ID NO: 97 - protein, 16 aa, synthetic construct
  REGION 1..16: note = Synthetic: MgPep
GNSFVGWCGAIGAPCA

SEQ ID NO: 98 - protein, 10 aa, synthetic construct
  REGION 1..10: note = Synthetic: MgPep
WCGAIGAPCA

SEQ ID NO: 99 - protein, 10 aa, synthetic construct
  REGION 1..10: note = Synthetic: PrPep
FCWRPGQPCG

SEQ ID NO: 100 - protein, 10 aa, synthetic construct
  REGION 1..10: note = Synthetic: PrPep
KWCGHIGQGC

SEQ ID NO: 101 - protein, 10 aa, synthetic construct
  REGION 1..10: note = Synthetic: HjPep
WCYRIGEPCW

SEQ ID NO: 102 - protein, 10 aa, synthetic construct
  REGION 1..10: note = Synthetic: HjPep
WCWILGGKCW

SEQ ID NO: 103 - protein, 10 aa, synthetic construct
  REGION 1..10: note = Synthetic: HjModl
WSYRIGEPCW

SEQ ID NO: 104 - protein, 10 aa, synthetic construct
  REGION 1..10: note = Synthetic: HjMod2
WCYRIGEPSW

SEQ ID NO: 105 - protein, 10 aa, synthetic construct
  REGION 1..10: note = Synthetic: AnPep
WCRFRGQVCG

SEQ ID NO: 106 - protein, 9 aa, synthetic construct
  REGION 1..9: note = Synthetic: BcPep
WCGRPGQPC

SEQ ID NO: 107 - protein, 9 aa, synthetic construct
  REGION 1..9: note = Synthetic: BcMod1
WSGRPGQPC

SEQ ID NO: 108 - protein, 9 aa, synthetic construct
  REGION 1..9: note = Synthetic: BcMod1
WCGRPGQPS

SEQ ID NO: 109 - protein, 10 aa, synthetic construct
  REGION 1..10: note = Synthetic: BbPep
WCMRPGQPCW

SEQ ID NO: 110 - protein, 9 aa, synthetic construct
  REGION 1..9: note = Synthetic: BbPep
WCMQTPKCW

SEQ ID NO: 111 - protein, 11 aa, synthetic construct
  REGION 1..11: note = Synthetic: NcPep
QWCRIHGQSCW

SEQ ID NO: 112 - protein, 14 aa, synthetic construct
  REGION 1..14: note = Synthetic: NcPep
QVCNMRLHPKKVCW

SEQ ID NO: 113 - protein, 10 aa, synthetic construct
  REGION 1..10: note = Synthetic: ShePep
YCPLKGQSCW

SEQ ID NO: 114 - protein, 12 aa, synthetic construct
  REGION 1..12: note = Synthetic: ShePep
QRYCPLKGQSCW

SEQ ID NO: 115 - protein, 11 aa, synthetic construct
  REGION 1..11: note = Synthetic: MoPep
QWCPRRGQPCW

SEQ ID NO: 116 - protein, 8 aa, synthetic construct
  REGION 1..8: note = Synthetic: DhPep
WCVYNSCP

SEQ ID NO: 117 - protein, 10 aa, synthetic construct
  REGION 1..10: note = Synthetic: FgPep
WCWWKGQPCW

SEQ ID NO: 118 - protein, 10 aa, synthetic construct
  REGION 1..10: note = Synthetic: FgPep
WCTWKGQPCW

SEQ ID NO: 119 - protein, 8 aa, synthetic construct
  REGION 1..8: note = Synthetic: peptide
PHGGGWGQ

SEQ ID NO: 120 - protein, 4 aa, synthetic construct
  REGION 1..4: note = Synthetic: peptide
KGHK

SEQ ID NO: 121 - protein, 4 aa, synthetic construct
  REGION 1..4: note = Synthetic: peptide
DAHK

SEQ ID NO: 122 - protein, 12 aa, Ebolavirus
  SITE 1..12: note = Ebolavirus (small secreted glycoprotein; Zaire)
VNATEDPSSGYY

SEQ ID NO: 123 - protein, 9 aa, Severe acute respiratory syndrome coronavirus 2
QIAPGQTGK

SEQ ID NO: 124 - protein, 9 aa, Ebolavirus
  SITE 1..9: note = Ebolavirus (small secreted glycoprotein; Zaire)
KTTKSWLQK

SEQ ID NO: 125 - protein, 9 aa, Ebolavirus
  SITE 1..9: note = Ebolavirus (small secreted glycoprotein; Zaire)
WLQKIPLQW

SEQ ID NO: 126 - protein, 12 aa, Ebolavirus
  SITE 1..12: note = Ebolavirus (VP40 protein; Zaire)
WTDDTPTGSNGA

SEQ ID NO: 127 - protein, 14 aa, Ebolavirus
  SITE 1..14: note = Ebolavirus (small secreted glycoprotein; Zaire, Sudan and Bundibugyo)
GFRSGVPPKVVNYE

SEQ ID NO: 128 - protein, 13 aa, Ebolavirus
  SITE 1..13: note = Ebolavirus (small secreted glycoprotein; Zaire, Sudan and Bundibugyo)
FHKEGAFFLYDRL

SEQ ID NO: 129 - protein, 14 aa, Ebolavirus
  SITE 1..14: note = Ebolavirus (small secreted glycoprotein; Zaire, Sudan and Bundibugyo)
KEGAFFLYDRLAST

SEQ ID NO: 130 - protein, 11 aa, Ebolavirus
  SITE 1..11: note = Ebolavirus (small secreted glycoprotein; Zaire, Sudan and Bundibugyo)
LPTQGPTQQLK

SEQ ID NO: 131 - protein, 9 aa, Ebolavirus
  SITE 1..9: note = Ebolavirus (small secreted glycoprotein; Zaire, Sudan and Bundibugyo)
QTEPKTSVV

SEQ ID NO: 132 - protein, 15 aa, Ebolavirus
  SITE 1..15: note = Ebolavirus (small secreted glycoprotein; Zaire)
SATKRWGFRSGVPPK

SEQ ID NO: 133 - protein, 13 aa, Ebolavirus
  SITE 1..13: note = Ebolavirus (VP40 protein; Zaire)
TGKKVTSKNGQPI

SEQ ID NO: 134 - protein, 17 aa, Severe acute respiratory syndrome coronavirus 2
  SITE 1..17: note = SARS-CoV-2 (nucleocapsid protein)
KHWPQIAQFAPSASAFF

SEQ ID NO: 135 - protein, 12 aa, Severe acute respiratory syndrome coronavirus 2
  SITE 1..12: note = SARS-CoV-2 (nucleocapsid protein)
WHWPQYGVFAPG

SEQ ID NO: 136 - protein, 12 aa, Severe acute respiratory syndrome coronavirus 2
  SITE 1..12: note = SARS-CoV-2 (nucleocapsid protein)
WHWPQIAVFAPS

SEQ ID NO: 137 - protein, 12 aa, Severe acute respiratory syndrome coronavirus 2
  SITE 1..12: note = SARS-CoV-2 (nucleocapsid protein)
KHWPQIAQFAPS

SEQ ID NO: 138 - protein, 18 aa, Severe acute respiratory syndrome coronavirus 2
  SITE 1..18: note = SARS-CoV-2 (S protein)
YQAGSTPCNGVEGFNCYF

SEQ ID NO: 139 - protein, 13 aa, Severe acute respiratory syndrome coronavirus 2
  SITE 1..13: note = SARS-CoV-2 (S protein)
GSTPCNGVEGFNC

SEQ ID NO: 140 - protein, 13 aa, Severe acute respiratory syndrome coronavirus 2
  SITE 1..13: note = SARS-CoV-2 (S protein)
GWIPRCGVEGSNC

SEQ ID NO: 141 - protein, 13 aa, Severe acute respiratory syndrome coronavirus 2
  SITE 1..13: note = SARS-CoV-2 (S protein)
GWIPCNGVEGFNC

SEQ ID NO: 142 - protein, 11 aa, Severe acute respiratory syndrome coronavirus 2
  SITE 1..11: note = SARS-CoV-2 (S protein)
GWIFGTTLDSK

SEQ ID NO: 143 - protein, 14 aa, Severe acute respiratory syndrome coronavirus 2
  SITE 1..14: note = SARS-CoV-2 (S protein)
GFQPTNGVGYQPYR

SEQ ID NO: 144 - protein, 12 aa, Severe acute respiratory syndrome coronavirus 2
  SITE 1..12: note = SARS-CoV-2 (S protein)
YGFQPTNGVGYQ

SEQ ID NO: 145 - protein, 12 aa, Severe acute respiratory syndrome coronavirus 2
  SITE 1..12: note = SARS-CoV-2 (S protein)
VYSTGSNVFQTR

SEQ ID NO: 146 - protein, 19 aa, Severe acute respiratory syndrome coronavirus 2
  SITE 1..19: note = SARS-CoV-2 (S protein)
FDNPVLPFNDGVYFASTEK

SEQ ID NO: 147 - protein, 23 aa, Severe acute respiratory syndrome coronavirus 2
  SITE 1..23: note = SARS-CoV-2 (S protein)
DLPQGFSALEPLVDLPIGINITR

SEQ ID NO: 148 - protein, 24 aa, Severe acute respiratory syndrome coronavirus 2
  SITE 1..24: note = SARS-CoV-2 (S protein)
VYSSANNCTFEYVSQPFLMDLEGK

SEQ ID NO: 149 - protein, 470 aa, Candida albicans
MNINSTFIPDKPGDIIISYSIPGLDQPIQIPFHSLDSFQTDQAKIALVMGITIGSCSMTLIFLISIMYKTNKLTNLKLKLKLKYILQWINQKIFTKKRNDNKQQQQQQQQQIESSSYNNTTTTTSGSYKLFLFYLNSLILLIGIIRSGCYLNYNLGPLNSLSFVFTGWYDGSSFISSDVTNGFKCILYALVEISLGFQVYVMFKTSNLKIWGIMASLLSIGLGLIVVAFQINLTILSHIRFSRAISTNRSEEESSSSLSSDSVGYVINSIWMDLPTILFSISINIMTILLIGKLIIAIRTRRYLGLKQFDSFHILLIGFSQTLIIPSIILVVHYFYLSQNKDSLLQQISLLLIILMLPLSSLWAQTANNTHNINSSPSLSFISRHHSSDSSRSGGSNTIVSNGGSNGGGGGGGNFPVSGIDAQLPPDIEKILHEDNNYKLLNSNNESVNDGDIIINDEGMITKQITIKRV

SEQ ID NO: 150 - protein, 403 aa, Candida parapsilosis
MNKIVSKLSSSDVIVTVTIPNEEDGTYEVPFYAIDNYHYSRMENAVVLGATIGACSMLLIMLIGILFKNFQRLRKSLLFNINFAILLMLILRSACYINYLMNNLSSISFFFTGIFDDESFMSSDAANAFKVILVALIEVSLTYQIYVMFKTPMLKSWGIFASVLAGVLGLATLATQIYTTVMSHVNFVNGTTGSPSQVTSAWMDMPTILFSVSINVLSMFLVCKLGLAIRTRRYLGLKQFDAFHILFIMSTQTMIIPSIILFVHYFDQNDSQTTLVNISLLLVVISLPLSSLWAQTANNVRRIDTSPSMSFISREASNRSGNETLHSGATISKYNTSNTVNTTPGTSKDDSLFILDRSIPEQRIVDTGLPKDLEKFINNDFYEDDGGMIAREVTMLKTAHNNQ

SEQ ID NO: 151 - protein, 347 aa, Baudoinia compniacensis
MASNGWQNNATFDPYAQTFVLLQPDGLTPFPALLGDVLALNTVSVTQGIIYGTQVGISGLLLLILLIMTKPDKRRSLVFILNSLSLLLIFARNVLSCVQLTTIFYNFYNWELHWYPESPALSRAMDLSAATEVLNIPIDVAIFSSLVVQVHIVCCTIHTLVRTSALLSSAAVGLAAVAVRFALAVVNIKYSIFGINTLTEPQFNLIVHLKRVSDILTVVAIAFFSSIFVAKLGVAIHTRRTLNLKNFGAIQIIFIMGCQTMLIPLIFVIVSFYASRGSQIGSMVPTVVATFLPLSGMWASAQTNNEKMGRADQRFHRAVPVGATDFSVTKARSAKASDTLDTLIGDD

SEQ ID NO: 152 - protein, 321 aa, Scheffersomyces stipites
MDTSINTLNPANIIVNYTLPNDPRVISVPFGAFDEYVNQSMQKAIIHGVSIGSCTIMLLIILIFNVKRKKSPAFYLNSVTLTAMIIRSALNLAYLLGPLAGLSFTFSGLVTPETNFSVSEATNAFQVIVVALIEASMTFQVFVVFQSPEVKKLGIALTSISAFTGAAAVGFTINSTIQQSRIYHSVVNGTPTPTVATWSWVRDVPTILFSTSVNIMSFILILKLGFAIKTRRYLGLRQFGSLHILLMMATQTLLAPSILILVHYGYGTSSNSQLILISYLLVVLSLPVSSIWAATANNSPQLPSSATLSFMNKTTSHFSES

SEQ ID NO: 153 - protein, 435 aa, Lodderomyces elongisporus
MDEAINANLVSGDIIVSFNIPGLPEPVQVPFSEFDSFHKDQLIGVIILGVTIGACSLLLILLLGMLYKSREKYWKSLLFMLNVCILAATILRSGCFLDYYLSDLASISYTFTGVYNGTSFASSDAANVFKTIMFALIETSLTFQVYVMFQGTTWKNWGHAVTALSGLLSVASVAFQIYTTILSHNNFNATISGTGTLTSGVWMDLPTLLFAASINFMTILLLFKLGMAIRQRRYLGLKQFDGFHILFIMFTQTLFIPSILLVIHYFYQAMSGPFIINMALFLVVAFLPLSSLWAQTANTTKKIESSPSMSFITRRKSEDESPLAANDEDRLRKFTTTLDLSGNKNNTTNNNNNSNNINNNMSNINYPSTGLGEDDKSFIFEMEPSRERAAIEEIDLGARIDTGLPRDLEKFLVDGFDDSDDGEGMIAREVTMLKK

SEQ ID NO: 154 - DNA, 1083 bp, Candida lusitaniae
  misc_feature 1..1083: note = Candida (Clavispora) lusitaniae (Cl)
  source 1..1083: mol_type = genomic DNA
atgaacccagctgacatcaacatcgaatacaccttgggtgatactgctttctcttccactttcgctgatttcgaagcttggaaaactagaaacactcaattcgctattgtcaacggtgtcgctttggcttgtggtattatcttgatggtcgtttcttggattattattgttaacaagagagctccaatcttcgctatgaaccaaactatgttggttatcatggttattaagtccgctatgtacttgaagcatatcatgggtccattgaactccttgaccttccgtttcaccggtttaatggaagaatcctgggctccatacaacgtttacgtcactattaacgtcttgcatgttttgttggtcgctgctgtcgaatcctctttggtcttccaaatccatgttgttttcaagtcttctagagccagagttgctggtagagccattgtttctgctatgtccactttggccttgttgatcgtttctttgtacttgtactctactgttagacatgctcaaactttgcgtgctgaattatctcatggtgacactaccactgttgaaccatgggtcgataacgttccattgattttgttttccgcttctttgaacgttttgtgtttgttgttggccttgaaattggttttcgctgtcagaaccagaagacatttaggtttaagacaattcgactctttccacatcttgattattatggccactcaaactttcgttatcccatcctctttggtcatcgctaactacagatacgcttcttccccattgttgtcttccatttccatcatcgtcgccgtctgtaacttgccattgtgttccttgtgggcttgttctaacaacaactcttcctacccaacttcttctcaaaacactattttgtccagatacgaaactgaaacctctcaagctactgacgcttcctctaccacctgtgccggtattgctgaaaagggtttcgacaagtctccagactctccaactttcggtgaccaagactccgtctctatctcccatatcttggactctttggaaaaggatgttgaaggtgtcaccacccatagattgacttag

SEQ ID NO: 155 - DNA, 1065 bp, Komagataella pastoris
  misc_feature 1..1065: note = Komagataella (Pichia) pastoris (Kp)
  source 1..1065: mol_type = genomic DNA
atggaagaatactccgactccttcgacccatcccaacaattgttgaacttcacttccttatacggtgaaaccgatgctactttcgctgaattggacgactaccacttctacgtcgttaagtacgccatcgtttacggtgccagaattggtgtcggtatgttttgtactttgatgttgttcgttgtttccaagtcttggaagactccaatcttcgtcttgaaccaatcttctttgattttgttgattattcactccggtttctacatccactacttgaccaaccaattctcttccttgacctacatgttcactagaatcccaaacgaaacccatgctggtgtcgatttgcgtattaacgtcgttaccaacaccttgtacgctttgttgatcttatctattgaaatttccttaatttaccaagtcttcgttatcttcaaaggtgtctacgaaaactctttaagatggattgttactattttcaccgctttattcgccgccgccgtcgttgctattaacttctacgtcactactttgcaatctgtctctatgtacaactctaacgttgactttccaagatgggcttctaacgtcccattgatcttgttcgcttcttctgtcaactgggcttgtttgttgttgtccttgaagttgttcttcgctatcaaggttagaagatctttgggtttgagacaattcgacacttttcacatcttggccatcatgttctctcaaactttgattatcccatccattttgattgtcttgggttacactggtaccagagacagagactccttggcttctttgggtttcttgttgatcgttgtttctttgccattttcctctatgtgggctgccactgctaacaactccaacatcccaacctctaccggttctttcgcctggaagaacagatactccccatctacttactccgacgataccactgctgtttccaagtccttcactattatgaccgctaaggatgaatgtttcaccactgataccgaaggttctccaagattcatcaagggtgacagaacctccgaagatttgcacttctag

SEQ ID NO: 156 - DNA, 1260 bp, Candida tenuis
  misc_feature 1..1260: note = Candida tenuis (Cn)
  source 1..1260: mol_type = genomic DNA
atggactcctacttgttgaaccatccaggtgacatctctttgaacttcgccttgccattgtccgatgaagtctacactattaccttcaacgacttagactctcaatcttctttttccattcaatacttggtcatccactcttgtgccattaccgtctgtttgaccttgttggttttgttgaacttgttcatcagaaacaagaagactccagtcttcgttttgaaccaagtcatcttgttcttcgctatcgtcagatcttctttgttcatcggttttatgaagtctccattgtccaccatcaccgcctctttcaccggtatcatttctgatgaccaaaaacacttctacaaggtctccgtcgctgctaacgccgctttgatcattttggtcatgttgattcaagtttctttcacttaccaaatctacattattttcagatccccagaagttagaaagttcggtgtcttcatgacctccgccttgggtgtcttgatggctgttaccttcggtttttacgttaactccgctgtcgcttctaccaagcaataccaacacatcttctactctaccgacccatacatcatggactcttgggtcactggtttgccaccaatcttgtactctgcttccgtcatcgctatgtctttggtcttggttttgaagttggtcgctgctgtcagaaccagaagatacttgggtttgaagcaattctcctcctaccacatcttgttgattatgttcacccaaaccttgttcgttccaaccatcttgaccatcttagcttacgctttctacggttacaacgatatcttgatccatatttctaccaccatcaccgttgtcttgttgccattcacctccatttgggcttctatcgccaacaactctagatccttgatgtctgccgcttccttgtacttctccggttccaactcctctttgtctgaattgtcttctccatctccatctgataacgacactttgaacgaaaacgtcttcgccttttttccagacaagttgcaaaagatgaactcttctgaagccgtttctgctgtcgacaaggtcgttgttcacgaccactttgataccatctcccaaaagtctatcccacacgacatcttggaaattttgcaaggtaacgaaggtggtcaaatgaaggaacacatctctgtctactctgatgactctttctccaagactactccaccaattgtcggtggtaacttgttgatcaccaacaccgacatcggtatgaagtag

SEQ ID NO: 157 - DNA, 1038 bp, Schizosaccharomyces japonicus
  misc_feature 1..1038: note = Schizosaccharomyces japonicus (Sj)
  source 1..1038: mol_type = genomic DNA
atgtactcctgggacgaattcagatccccaaagcaagctgaagttttgaaccaaaccgttaccttggaaactattgtttccaccattcaattgccaatctctgaaattgactccatggaaagaaacagattgttgaccggtatgactgtcgctgttcaagttggtttaggttccttcattttagttttgatgtgtattttctcttcctctgaaaagagaaagaagccagtcttcatcttcaacttcgctggtaacttggttatgactttgagagctattttcgaagttatcgttttggcttctaacaactactctatcgctgttcaatacggtttcgcttttgctgccgtcagacaatacgttcacgccttcaacattatcatcttgttgttgggtccattcatcttgttcatcgctgaaatgtctttgatgttgcaagttagaatcatttgttcccaacacagaccaactatgattaccaccactgttatctcttgtattttcactgttgttaccttggccttctggatcaccgacatgtctcaagaaattgcttaccaattgttcttgaaaaactacaacatgaagcaaattgttggttactcctggttgtactttatcgctaagatcaccttcgctgcttccattatcttccattcctccgtcttctccttcaaattgatgcgtgctatttacattcgtagaaagatcggtcaattcccattcggtccaatgcaatgtatcttcattgtttcctgtcaatgtttgatcgttccagctattttcactttgatcgattctttcacccacacttacgatggtttctcctccatgactcaatgtttgttgatcatctccttaccattgtcttccttgtgggccacccacaccgctcaaaagttgcaaaccatgaaggataacactaacccaccatctggtacccaattaaccatcagagttgatcgtactttcgacatgaagttcgtttccgactcctctgacggttctttcactgaaaagaccgaagaaactttgcca

SEQ ID NO: 158 - DNA, 1197 bp, Baudoinia compniacensis
  misc_feature 1..1197: note = Baudoinia compniacensis (Bc)
  source 1..1197: mol_type = genomic DNA
atggcttctaactcttctaacttcgacccattgactcaatctatcactatcttgatggctgacggtatcactactgtttctttcactccattggacatcgacttcttctactactacaacgttgcttgttgtatcaactacggtgctcaagctggtgcttgtttgttgatgttcttcgttgttgttgttttgactaaggctgttaagagaaagactttgttgttcgttttgaacgttttgtctttgatcttcggtttcttgagagctatgttgtacgctatctacttcttgcaaggtttcaacgacttctacgctgctttcactttcgacttctctagagttccaagatcttcttacgcttcttctgttgctggttctgttatcccattgtgtatgactatcactgttaacatgtctttgtacttgcaagcttacactgtttgtaagaacttggacgacatcaagagaatcatcttgactactttgtctgctatcgttgctttgttggctatcggtttcagattcgctgctactgttgttaactctgttgctatcttggctacttctgcttcttctgttccaatgcaatggttggttaagggtactttggttactgaaactatctctatctggttcttctctttgatcttcactggtaagttggtttggactttgtacaacagaagaagaaacggttggagacaatggtctgctgttagaatcttggctgctatgggtggttgtactatggttatcccatctatcttcgctatcttggaatacgttactccagtttctttcccagaagctggttctatcgctttgacttctgttgctttgttgttgccaatctcttctttgtgggctggtatggttactgacgaagaaacttctgctatcgacgtttctaacttgactggttctagaactatgttgggttctcaatctggtaacttctctagaaagactcacgcttctgacatcactgctcaatcttctcacttggacttctcttctagaaagggttctaacgctactatgatgagaaagggttctaacgctatggaccaagttactactatcgactgtgttgttgaagacaaccaagctaacagaggtttgagagactctactgaaatggacttggaagctatgggtgttagagttaacaagtcttacggtgttcaaaaggcttag

SEQ ID NO: 159 - DNA, 1239 bp, Beauvaria bassiana
  misc_feature 1..1239: note = Beauvaria bassiana (Bb)
  source 1..1239: mol_type = genomic DNA
atggatggttcttctgctccatcttctccaactccagatccaaccttcgacagattcgccggtaacgtcactttcttcttggctgaccacatcaccactacctccgttccaatgccagtcttgaacgcctactacgacgaatccttgtgtactaccatgaactacggtgctcaattaggtgcttgtttagttatgttggttgtcgttgttgctttgaccccagctgctaagttggctagaagaccagcttctgctttgcatttggttggtttgttgttgtgtgctgttagatccggtttgttgtttgcttacttcgtctccccaatctctcacttttaccaagtttgggctggtgacttctctgccgtttccagaagatactgggacgcttctttggctgccaacactttagctttcccattggttgtcgtcgttgaagctgctttgatcaaccaagcttggaccatggttgctttctggccaagagccgctaaggccgctgcctgtgcttgttctgctgtcattgtcttgttgactattggtactagattggcctacactatcgtccaaaaccacgctattgttactgccgtcccaccagaacacttcttgtgggctattcaatggtccgctgttatgggtgctgtttccatcttctggttttgtgccgttttcaacgtcaagttggtctgtcacttagtcgctaacagaggtatcttgccatctatctctgttgttaacccaatggaagtcttggttatgactaacggtaccttgatgattatcccatctatcttcgctggtttggaatgggctaagttcaccaacttcgaatccggttctttgactttgacttccgttattattatcttgccattgggtactttggctgcccaacgtatttctggtcaaggttcccaaggttaccaagctggtcacttattccacgaacaacaacaacaacaagctcgtacccgttccggtgccttcggttccgcttctcaacaatcccatccaactaacaaggttccatcctctattaccttgtctacctctggtactccaattactccacaaatctctgccggttcccgtccagaattaccattggttgatagatccgaacgtttggacccaattgacttggaattgggtagaatcgatgctttcagaggttcttccgacttctctccatccaccgctagaccaaagcgtatgcaacgtgataacttcgcctag

SEQ ID NO: 160 - DNA, 891 bp, Hypocrea jecorina
  misc_feature 1..891: note = Hypocrea jecorina (Hj)
  source 1..891: mol_type = genomic DNA
atgtcttccttcgacccatacactcaaaacattactattttggtttctccatcctctccaccaatttccattccaatcccagttatcgacgctttcaacgacgaaaccgcttctatcattactaactacgccgctcaattaggtgctgctttggccatgttattagttttgttggccgctactccaaccgctagattgttaagagctgatggtccatccttgttgcacgctttggccttgttagtctgtgtcgtcagaactgtcttattgatctacttcttcttgaccccattctctcacttctaccaagtctggaccggtgacttctctcaagttccagcttggaactacagagcttctattgctggtaccgttttgtctactttgttgaccgttgttaccgacgctgctttggttaaccaagcttggactatggtttctttattcgctccaagaactaagagagccgtttgtgttttgtccttgttaatcaccttgttggccatttctttcagagtcgcttacaccgtcattcaatgtgaaggtatcgctgaattggctgctccaagacaatacgcttggttgatcagagccactttgatctttaacatctgttccattgcctggttctgtgctttgttcaactctaagttggttgctcacttggttaccaacagaggtgtcttgccatcccgtagagccatgtccccaatggaagttttgattatggccaacggtatcttgatgattgttccagttgttttcgctatcttggaatggcaccacttcattaacttcgaagctggttctttaaccccaacctccatcgccattatcttgccattgtcctctttggccgcccaaagaatcgccaacacttcttcctcttag

SEQ ID NO: 161 - DNA, 1407 bp, Sporothrix scheckii
  misc_feature 1..1407: note = Sporothrix scheckii (She)
  source 1..1407: mol_type = genomic DNA
atgaaacccgccgctggacctgcatctagtccattcgacccatttaaccaaacgttttacctgaccggtccagataataccactgtaccagtctcagtcccacaagttgactatatctggcattatattattggaacatccatcaactatggttctcagatcggagcctgtttacttatgcttcttgtgatgttgacattgacttcaaagtcaagattttctcgtgcggccactctgattaacgtagcaagcttattgattggagtaattcgttgtgttcttttagctgtctactttacttcttctctaactgaattgtatgctctgttcgttggcgattacagccaggtccgtaggtctgatctttgtgtctctgctgtggcaaccttctttagtctaccacaattagttctaatagaagctgctttgtttctacaggcttatagtatgatcaaaatgtggccatccctgtggagagcagtggttttagctatgtcagtggtggtggctgtgtgtgcaatcggttttaagttcgcgtccgttgttatgcgtatgaggtcaacattaacattggacgattctttggatttctggctagtggaagtcgatctggcttttacagcaactactattttttggttttgtttcatctacattataaggttggttattcatatgtgggaatatagaagcattttaccaccaatggggtctgtttctgctatggaggttcttgttatgaccaatggagcgttgatgttagttccagtgattttcgccgcaatagaaatcaatggtttatcaagctttgaatcagggtcactggttcatacatcagtgattgtattattacctttaggtagcttgatagcgcaagcaatgacacgtccagatgggtatgtccaaagaacgaatacatctggagcatcaggcgcaagtggtgcacatcctggtagaaatggatccggacacggtggtcatggtggtgcgtactcaagagccatgactaataccctaaatacattggatacattggataccgtagacagtaagacatccataatgcatcatcatcatcaccatcatagaaaccactcaaatggcatgagtaagacgaaggcaaatagtggaacatggagccatgcgtcagatgctaactccaccaatgctatgatcagcggtggtatcgcaactcaagttaggattcaagctaatcagtcaaccttaggaaatacggggatgtccgggggctctggagcccctaattctcatactcgtaataactcattggctgctatggaaccagtggagaagcaactgcatgatatcgatgccacacctttaagcgcatctgattgcagggtctgggttgatcgtgaggtcgaggtcagaagggacatggtctag

SEQ ID NO: 162 - DNA, 1194 bp, Pseudogymnoascus destructans
  misc_feature 1..1194: note = Pseudogymnoascus destructans (Pd)
  source 1..1194: mol_type = genomic DNA
atgtccactgccaacgttcatttaccagctgatttcgatccaactagacaaaacatcactatctataccccagacggtaccccagttgttgctaccttgccaatgatcaatttgtttaacagacaaaacaacgaaatctgtgttgtttacggttgtcaattgggtgcctctttaattatgttcttggttgttttgttgaccaccagagtttccaagagaaaatctccaatcttcgtcttgaacgttttgtctttgattatttcttgtttaagatccttgttgcaaattttatactatattggtccatggaccgagatctacagatacttgtctttcgattactctactgtcccagcttccgcttacgctaattctgttgctgccactttattaaccttattcttattgattaccattgaagcttctttagttttacaaactaacgttgtctgcaagtctatgtcttctcacattcgttggccagttactgctttgtccatggttgtctctttattggctatttcttttagattcggtttgaccatccgtaacatcgaaggtatcttaggtgctactgtcaaatccgactccttaatgttctctggtgcctctttgatctctgaaactgcttctatctggttcttctgcactattttcgttattaaattgggttggaccttgtaccaaagaaagaagatgggtttgaagcaatggggtccaatgcaaattatcactatcatggctggttgcaccatgttgatcccatccttgttcactgttttggaattcttccctgaagaaactttctacgaggccggtactttggctatctgtttggttgctattttgttgccattatcttccgtctgggctgccgctgctattgatggtgatgaaccagtccgtccacatggttctaccccaaaattcgcttctttcaacatgggttccgactacaaatcttcttctgctcacttgccaagatctattagaaaggcctccgtcccagctgaacatttatctagaacttctgaagaagagttaggtgacgacggtactttgaacagaggtggtgcctacggtatggacagaatgtccggttctatctcccctagaggtgtcagaattgaaagaacttacgaagttcataccgctggtagaggtggttctatcgagagagaggacatcttctag

SEQ ID NO: 163 - DNA, 1308 bp, Lodderomyces elongisporous
  misc_feature 1..1308: note = Lodderomyces elongisporous (Le)
  source 1..1308: mol_type = genomic DNA
atggacgaagcaatcaatgcaaaccttgtttctggagatattatagtctcttttaacattcctggtttgccagaaccggtacaagtgccattcagcgaatttgattcgtttcataaagaccagctcattggagtcatcattcttggagtcactattggagcatgctcgcttttgttgatattgctacttggaatgttatacaagagccgtgaaaagtattggaaatcactattatttatgctcaatgtatgcatcttggctgccacaatcttaaggagcggttgcttcttagactattatctaagtgatttggccagtatcagttatacatttactggagtatacaatggtaccagctttgctagctctgacgcggcaaatgtgttcaagactattatgtttgccttgattgaaacttcgttaacctttcaagtgtatgtcatgtttcaagggaccacttggaaaaattggggccatgctgtcactgcattatcgggtctcttgtctgttgcctcagtggcgttccagatctacaccacgattttatcccacaataatttcaatgctacaatctcgggaaccggtacattaacttcaggtgtttggatggacttaccaacactcttgtttgccgcaagtatcaattttatgaccattttgttgttatttaagttgggaatggccattagacaaagaaggtatttaggtttaaaacagtttgatgggttccatatcttattcatcatgtttacccaaacattgttcataccctcgattttgcttgtgatccactacttttaccaggcaatgtctggaccattcatcatcaacatggcgttgttcttggtggtggcattcttgccattgagttcattatgggcacaaactgcaaacactactaaaaagattgaatcttcgccaagtatgagctttattactagacgaaaatcagaggatgagtcaccactggctgctaacgacgaggataggttacgaaaattcaccacaactttggatttgtcgggcaacaagaacaatacaacaaacaataataacaatagcaacaacattaacaacaatatgagcaacatcaactacccttctacaggactgggagaagacgataaatcctttatatttgagatggaacccagtcgggaaagagctgcaatagaagagattgatcttggagcaaggatcgataccggtttgcccagagatttagagaaatttctagttgatgggtttgacgatagtgatgacggagaaggaatgatagccagagaagtgactatgttgaaaaaatag

SEQ ID NO: 164 - DNA, 1143 bp, Penicillium chrysogenum
  misc_feature 1..1143: note = Penicillium chrysogenum (Pr)
  source 1..1143: mol_type = genomic DNA
atggctacctcttccccaatccaaccatttgacccattcacccaaaacgttaccttccgtttgcaagacggtaccgaattcccagtttctgtcaaggctttggacgtcttcgtcatgtacaacgttagagtctgtattaactacggttgtcaattcggtgcctccttcgtcttgttagtcattttagtcttgttaactcaatccgacaagagaagatctgctgtcttcattttgaacggtttggctttgttcttgaactcttctagattgttgtttcaagttattcacttctccactgccttcgaacaagtctacccatacgtctctggtgactactcctctgtcccatggtccgcttacgctatctccattgtcgctgttgttttgactaccttggtcgttgtttgtatcgaagcttctttggttattcaagttcacgttgtctgttccaccttgagacgtagatacagacacccattattagctatttctattttggtcgctttggttccaatcggtttcagatgtgcttggatggtcgctaactgtaaggctattattaaattgacctacaccaacgacgtttggtggatcgaatctgctactaacatctgtgtcactatctccatctgtttcttctgtgttatcttcgttaccaagttgggtttcgccatcaagcaaagaagaagattgggtgttagagaattcggtccaatgaaggttattttcgtcatgggttgtcaaactatggttgttccagctattttctccatcacccaatactacgtcgtcgtcccagaattctcctctaacgtcgttactttggttgtcatttctttaccattatcttccatttgggccggtgctgtcttggaaaacgctagaagaaccggttcccaagatagacaaagaagacgtaacttgtggagagctttggttggtggtgctgaatccttgttatccccaactaaggactctccaacctctttgtctgctatgactgctgctcaaaccttatgttactctgatcacaccatgtccaagggttctccaacttccagagacaccgatgctttctacggtatctccgttgaacacgacatctccattaacagagttcaacgtaacaactccatcgtctag

SEQ ID NO: 165 - DNA, 1110 bp, Aspergillus oryzae
  misc_feature 1..1110: note = Aspergillus oryzae (Ao)
  source 1..1110: mol_type = genomic DNA
atggactctaagttcgacccatactctcaaaacttgactttccacgctgctgacggtaccccatttcaagttccagtcatgaccttgaacgacttttaccaatactgtattcaaatttgtatcaactacggtgctcaattcggtgcttccgtcatcattttcattatcttgttgttattgactagaccagacaaaagagcttcttctgttttcttcttaaacggtggtgccttgttgttgaacatgggtagattgttgtgtcacatgatttacttcactactgacttcgtcaaggcttaccaatacttctcttctgattactctagagccccaacctctgcctacgctaactccattttgggtgtcgtcttgaccaccttgttgttggtttgtatcgaaacctccttggttttacaagtccaagtcgtctgtgctaacttgagacgtagatacagaaccgtcttattgtgtgtttctatcttggtcgccttgatcccagtcggtttgagattgggttacatggttgaaaactgtaagactattgttcaaactgataccccattgtctttggtttggttggaatctgctactaacatcgtcattaccatctccatctgtttcttctgttctatcttcatcatcaagttgggtttcgccattcaccaaagaagaagattgggtgtcagagatttcggtccaatgaaggtcattttcgtcatgggttgtcaaactttgactgttccagctttgttgtctattttgcaatacgctgtctctgtcccagaattgaactctaacattatgactttggttactatctctttgccattgtcctccatttgggctggtgtttctttgacccgttcttcctccaccgaaaactctccatccagaggtgctttgtggaaccgtttgaccgactctaccggtaccagatctaaccaaacctcttccaccgacaccgccgtcgctatgacctacccatctaacaagtcttctactgtctgttacgccgatcaatcttctgtcaagagacaatacgatccagaacaaggtcacggtatctctgttgaacacgatgtttctgtccactcctgtcaaagattgtag

SEQ ID NO: 166 - DNA, 1071 bp, Paracoccidioides brasiliensis
  misc_feature 1..1071: note = Paracoccidioides brasiliensis (Pb)
  source 1..1071: mol_type = genomic DNA
atggcaccctcattcgaccccttcaaccaaaacgtggtcttccacaaggccgacggaaccccattcaacgtctcaatccatgaactagacgatttcgtgcagtacaataccagagtctgcatcaactactcttcccagctcggagcatctgtcattgcgggactcatgcttgccatgctgacacactcagaaaagcgtcgtctgccggttttcttcctaaacacattcgcactggccatgaactttgcccgcctgctctgcatgaccatctacttcaccacgggcttcaacaagtcctatgcctactttggtcaggattactcccaggtgcctgggagcgcctacgcagcctctgtcttgggcgttgtcttcaccactctgctggtaatcagcatggaaatgtccctcctgatccaaacgagggttgtctgcacgacccttccggatatccaacgttatctactcatggcagtttcctccgcgatttccctgatggccatcgggttccgccttggcttaatggttgagaactgcattgctattgtgcaggcgtcgaatttcgcgccttttatctggcttcaaagcgcctcgaacatcaccattacgatcagcacatgtttcttcagtgccgtctttgttacgaaattggcatatgcactcgtcactcgtatacgactaggcttgaccaggtttggtgctatgcaggttatgtttatcatgtcctgccagactatggtgatcccagccatcttctcaattctccaatacccactccccaagtacgaaatgaactccaacctctttacgctggtggccattttccttcctctttcctcgctatgggcttcagtggctacgaaatccagtttcgagacgtcttcttccggccgccatcagtatctttggccaagcgaacagagcaataacgtcaccaattcggaaattaagtatcaggtcagcttctctcagaaccacactacgctgcggtctggagggtctgtggccacgacactctctccggaccggctcgacccggtttattctgaagttgaagctggcacaaaggcctag

SEQ ID NO: 167 - DNA, 1110 bp, Aspergillus fischeri
  misc_feature 1..1110: note = Aspergillus fischeri (Af)
  source 1..1110: mol_type = genomic DNA
atgaactccaccttcgacccatggacccaaaacattactttgactcaatccgacggtaccactgtcatctcctctttggctttggccgatgactacttgcactacatgattagattgggtatcaactacggtgcccaattgggtgcttgtgctgttttgttgttggttttgttattgttgactagaccagaaaagagagtttcttctgtcttcgttttgaacgtcgctgctttgttggctaacatcatcagattgggttgtcaattgtcctacttctctaccggtttcgctagaatgtacgccttgttggccggtgacttctccagagtctctcgtggtgcttacgccggtcaagttatggcctccgtcttcttcaccattgtcttcatttgtgttgaagcttctttggttttgcaagttcaagtcgtctgttctaacttgagaagacaatacagaatcttgttattgggtgcttccactttggctgccttggttccaattggtgttcgtttgacttactccgttttaaactgtatggttattatgcacgctggtactatggaccacttggattggttggaatctgctaccaacatcgttactaccgtttctatttgtttcttctgtgctgttttcgttgtcaaattaggtttggctatcaagatgagaaagcgtttgggtgtcaaacaattcggtccaatgagagttatcttcatcatgggttgtcaaaccatgaccatcccagctattttcgctatttgtcaatacttctctagaattccagaattttctcataacgttttgactttggttatcatctctttgccattgtcttctatctgggccggttttgctttggtccaagccaactctaccgccagatctaccgaatctagacatcatttgtggaacattttgtcttccgatggtgctaccagagacaagccatcccaatgtgtttcttctccaatgacctctccaaccactacctgttactccgaacaatccacctctaagccacaacaagacccagaaaacggttttggtatttctgttgcccacgatatttccatccactctttcagaaaggacgcccacggtgatatttag

SEQ ID NO: 168 - DNA, 1110 bp, Yarrowia lipolytica
  misc_feature 1..1110: note = Yarrowia lipolytica (Yl)
  source 1..1110: mol_type = genomic DNA
atgcaattgccaccacgtccagacttcgacattgccactttggttgcctctatcactgttccagaaactgaattggtcttgggtcaaatgccattgggtgctttagaacaattgtaccaaaacagattgcgtttggctattttgttcggtgtcagagtcggtgctgctgttttgaccttgattgctatgcacttaatctccaagaagaacagaaccaagatcttgttcttggctaaccaaatgtctttgatcatgttgatcatccatgctgctttgtacttcagattcttgttgggtccattcgcctccatgttgatgatggttgcttacatcgttgatccaagatctaacgtctctaacgatatctctgtttctgttgccaccaacgttttcatgatgttgatgattatgtccgtccaattgtctttggctgttcaaacccgttctgttttccacgcttggttgaagtctcgtatttacgttaccgttggtttaatcttgttgtccttggtcgtcttcgtcttctggaccacccacactatcgtttcttgtatcgttttaacccatccaactagagacttgccatctatgggttggactagattagcttctgacgtttccttcgcttgttctatctctttcgcttctttggtcttgttggctaagttggtcaccgccatcagagttagaaagaccttgggtaagaagccattgggttacaccaaggttttggtcatcatgtccactcaatctttagtcgttccatctatcttgattatcgttaactacgctttgccagaaaaaaactcttggatcttgtctggtgtcgcttacttgatggttgttttgtccttaccattgtcctccatttgggctaccgccgtccatgacgacgaaatgcaatccaactacttgttgtctgccttgaaagatggtcacgttcaaccatccgaatctaagttgaagactgttttcttgaacagattgagaccattctctactaccactaacagagacgatgaatcctctgttgattccccagccatgccatctccagaatctgatgttaccttcttgaacactggtttcgaatgtgacgaaaagatgtag

SEQ ID NO: 169 - DNA, 1104 bp, Tuber melanosporum
  misc_feature 1..1104: note = Tuber melanosporum (Tm)
  source 1..1104: mol_type = genomic DNA
atggagcaaatcccagtctacgagcgtccaggtttcaacccacacaagcaaaacattaccttgttcaagcatgatggttctactgttactgtcggtttgcatgagttggacgccatgttcactcattccatcagagttgctgtcgtcttcgcctctcaaattggtgcttgtgctttgttgtctgttatcgttgctatggtcaccaagagagaaaagagacgtgctttgttcttcttgcacattatttccttgttgttggtcgttgttcgttccgtcttgcaaatcttgtacttcgtcggtccatgggctgaaacttataattacgtcgcctactactatgaagacattcctttgtctgacaaattgatttccatttgggctggtattatccaattgattttgaatatctgtattttgttatctttgatcttgcaagttcgtgtcgtttacgccacctctccaaaattgaacactattatgactttagtctcttgtgttatcgcttctatttctgtcggtttcttctttactgtcatcgttcaaatttctgaggctattttaaacggtgttggttacgacggttgggtttacaaagtccatagaggtgtcttcgctggtgctatcgccttcttctctttcatcttcatctttaagttggccttcgctatcagaagaagaaaggctttgggtttgcaaagattcggtccattgcaagttatcttcatcatgggttgtcaaactatgattgttccagctatctttgctactttggaaaacggtgttggtttcgaaggtatgtcctctttgactgctaccttggctgtcatttccttaccattgtcttctatgtgggccgccgctcaaaccgacggtccatctccacaatccactccaagagacggttatagaagattctctactcgtagatctgccttgaacagatctgacccatctggtggtagatctgttgacatgaacaccttggactctaccggtaacgattccttagctttgcacgttgataagacttttactgttgaatcttccccatcctcccaatctcaagctggtccacacaaggaaagaggtttcgaattcgcctag

SEQ ID NO: 170 - DNA, 1584 bp, Dactylellina haptotyla
  misc_feature 1..1584: note = Dactylellina haptotyla (Dh)
  source 1..1584: mol_type = genomic DNA
atggaccacaacacccaacacttcaacagacctgaatacattgaaatcccagttccaccatctaagggtttcaacccacacaccaaccctgctttcttcatctacccagacggttctaatatgaccttttggttcggtcaaatcgacgatttcagacgtgaccaattattcactaacaccatcttttccattcaaattggtgccgctttggtcatcttatgtgtcatgttttgtgttacccacgctgataagcgtaaaaccattgtctacttgttaaacgtttccaacttgttcgttgttatcattagaggtgttttctttgttcattacttcatgggtggtttggccagaacctataccactttcacctgggatacttctgatgttcaacaatctgagaaggctacttccattgtctcctctatttgttctttgattttgatgatcggtactcaaatctccttattgttgcaagtcagaatctgttacgctttgaacccaagatccaagaccgctatcttggttacttgtggttctatttccggtattgctaccactgcttatttattgttgggtgcttacactattcaattgagagaaaagccaccagacatgaagttcatgaagtgggctaagccagttgttaacgctttggttgccttgtccattgtctccttttctggtattttctcttggagaatgttccaatctgtcagaaacagaagaagaatgggtttcactggtatcggttccttggaatctttgttggcttctggtttccaatgtttagtcttccctggtttggttactaccgctttgaccgtcgccggttccacttggtatatcgctgttaacttaactactccatctgacttgaccgctatttacaactgttccgcttttttcgcttatgctttctccattccattgttaaaggaaagagctcaagttgaaaagaccatttctgttgtcattgctatcgctggtgtcttagtcgttgcttacggtgacggtgctgacgacggttccacctctaacggtgaaaaggctagattgggtggtaacgtcttgatcggtatcggttctgtcttgtatggtttatacgaagtcttgtataagaagttattatgtccaccatctggtgcttccccaggtagatctgttgttttctctaataccgtttgtgcttgcatcggtgctttcactttgttattcttgtggatcccattgccattgttgcactggtccggttgggaaatttttgaattgccaaccggtaagactgctaagttattgggtatttccattgccgctaacgccaccttctctggttctttcttgatcttaatttctttgactggtccagttttgtcctctgttgccgccttgttgaccattttcttggttgctattactgacagaattttattcggtagagaattgacttctgctgccattttgggtggtttgttgatcatcgctgccttcgctttgttatcttgggctacttggaaggaaatgattgaagagaacgagaaggatactatcgattccatctctgacgttggtgaccacgatgactag

SEQ ID NO: 171 - DNA, 1185 bp, Candida guilliermondii
  misc_feature 1..1185: note = Candida (Pichia) guilliermondii (Cgu)
  source 1..1185: mol_type = genomic DNA
atgaagtcctgctccatcggtttcggtatcccattcattaatgaaccaaacttcgaaactgtttctattttgaccatggacgtttctttcattgacgctgacgtcaatcctgacaatatcttgttgaacttcaccattcctggttaccaaaacggtttctctgttccaatggttgttattaacgaattgcaaaagtctcaaatgaaatacgctattgtttacggttgtggtgtcggtgcctccttgattttgttgtttgtcgtctggattttgtgttctagaaagactccattgtttatcatgaacaacattccattagttttgtacgtcatctcctcttctttgaacttggcttacattaccggtccattgtcttctgtttccgtcttcttgaccggtatcttgacttctcacgatgccattaacgtcgtttacgcttccaacgctttgcaaatgttgttgatcttttctatccaatctaccatggcctaccacgtttacgttatgttcaaatctccacaaattaaatacttgagatacatgttagtcggtttcttgggttgtttacaaattgtcaccacctgtttatacatcaactacaatgttttgtactctcgtagaatgcacaaattgtacgaaactggtcaaacctaccaagatggtaccgttatgactttcgttccattcatcttgttccaatgttctgtcaacttctcttctattttcttggttttgaagttgattatggccattagaaccagacgttacttgggtttgcgtcaattcggtggttttcatattttgatgatcgtttctttacaaactatgttggtcccatctattttggttttggttaactacgccgctcataaggctgttccttccaacttgttatcttccgtttctatgatgatcattgttttgtctttaccagcttcttctatgtgggccgctgctgctaacgcctcttctgccccttcctccgctgcttcctccttgttcagatacaccacttctgattccgatagaactttggaaactaaatctgaccacttcatcatgaagcatgagtcccacaactcttctccaaattcctccccattgactttggttcaaaagagaatttctgatgccaccttagaattaccaaaagagttagaagacttgatcgactccacctccatc

SEQ ID NO: 172 - DNA, 1293 bp, Aspergillus nidulans
  misc_feature 1..1293: note = Aspergillus nidulans (An)
  source 1..1293: mol_type = genomic DNA
atggctacccacaaccaaatctctgatcaatgtcaatggtcttacccagaagtcttcaccactcaagctgtcgaagaaccaaccgccgaaccagcttcttaccacttgcactctaccttgactattatggcttctaacttcgacccatggaaccaaaccattaccttcagattggaagacggtactccattcgacatttctgtcgactacttggacggtatcttgcaatactctatcagagcttgtgtcaactacgctgctcaattgggtgcttctgtcattttgtttgttatcttggtcttgttgactagagccgaaaaaagagcttcttgtttgttctggttaaactccttagctttgttgttgaacttcgccagattgttgtgtgacgtcttgttcttcaccggtaacttcgtcagaatttacactttgatctccgctgacgaatctagagttactgcttccgacttggctacttccatcgtcggtgctatcatgaccgctttgttgttgaccactattgaaatttctttggttttgcaagtccaagtcgtttgttctaacttgagaagaatctacagaagagccttgttgtgtgtttccgccgtcgttgccactgctaccattgctattagatactccttgttggctgtcaacattagagctattttggaattctccgacccaactacttacaactggttggaatctttagctaccgtcgccttgaccatctccatctgttacttctgtgtcatcttcgtcaccaagttaggtttcgctattagattgagaagaaagttgggtttatctgaattgggtccaatgaaggtcgtcttcatcatgggttgtcaaaccttggtcatcccaggtaaaagaaccttgtcttctttgattccaccagtcattgtttctattactcactacgtctccgacgtcccagaattgcaaactaacgttttgactatcgtcgccttgtccttgccattgtcctctatttgggctggtaccaccattgacaagccagtcactcactctaacgttagaaacttgtggcaaatcttgtccttctctggttacagaccaaagcaatctacctacattgctaccactactaccgctactaccaacgctaagcaatgtacccactgttactctgaatctagattgttgactgaaaaggaatctggtcgtaacaacgacacttcttctaagtcttcctcccaatacggtatcgctgtcgaacacgatatttccgttagatctgctcgtcgtgaatcttttgacgtctag

SEQ ID NO: 173 - DNA, 1332 bp, Zygosaccharomyces rouxii
  misc_feature 1..1332: note = Zygosaccharomyces rouxii (Zr)
  source 1..1332: mol_type = genomic DNA
atgagtgagattaacaattctacctacaatccaatgaatgcatatgtaacgtttacatcaatatatggtgatgatactatggtacgtttcaaagatgtggaattggtagttaacaaaagggttacagaagccattatgttcggcgtcaaagttggtgcagcttcgttgacactcatcatcatgtggatgatctctaagaaaagaacaacaccgatatttatcataaatcagtcttcgcttgtatttaccataatacatgcttcgctttattttgggtaccttttgtcaggatttggtagtatagtttacaatatgacatcgttcccgcagttaataagctccaatgacgttcgtgtgtacgcagctacaaatatttttgaggtcctgttggtagcatctatcgaaatctctctggtttttcaggtcaaagttatgtttgccaacaataatggtcgaagatggacttggtgtttgatggtagtttccatagggatggcactagctactgtaggactttattttgccactgccgttgagttgatcagagctgcttacagcaatgatactgttagccgccatgttttttacaatgtttctctgatcttactagcgtcatctgtcaatctaatgacactaatgctagtggtaaaattagtattagcgatcagatcaagaagatttttggggttaaaacagtttgacagtttccacatattacttataatgtcttgccagactctaatagcaccttccattctattcattttgggttggaccttagaccctcatactggtaatgaggttttaattacagttggtcaattgctaatagtactgtcattaccgctgtcatctatgtgggctacaaccgctaacaataccagttcatctagtagttcggtgtcctgtaatgacagctcttttggtaatgacaatctctgttccaagagttcgcaatttagaagaacttttatgaatagattccgtcccaagtcggttaatggtgacggtaattctgaaaatacctttgttacaattgatgatttggaaaaaagcgtttttcaagaattatcaacacctgttagcggagaatcaaagatagatcatgatcatgcaagtagtatttcatgtcaaaagacatgtaatcatgttcatgcttcgacagtgaattcagataagggatcttggtcctctgatggtagttgtggcagttctccgttaagaaagacttccaccgttaattctgaagatttacctccacatatattgagcgcctacgatgacgatcgaggtatagtagaaagtaaaaaaattatcctaaagaaattatag

SEQ ID NO: 174 - DNA, 1308 bp, Geotrichum candidum
  misc_feature 1..1308: note = Geotrichum candidum (Gc)
  source 1..1308: mol_type = genomic DNA
atggccgaagactccatcttcccaaacaactccacctctccattgaccaacccaattgttgttgaaaccattaagggtaccgcttacattccattacactacttggatgatttgcaatacgaaaagatgttgttggcttccttgttctccgttagaattgctacttccttcgttgttattatttggtacttcgtcgctgtcaacaaggctaagagatctaagtttttgtacattgtcaaccaagtttctttgttgatcgtttttatccaatccattttgtctttgatttacgtcttctccaacttctccaagatgtctaccattttgaccggtgattacaccggtatcactaagagagacattaacgtctcttgtgttgcctccgttttccaattcttgttcatcgcttgtatcgaattggctttgttcatccaagctactgtcgttttccaaaaatctgttagatggttgaagttttccgtttctttgatccaaggttccgtcgctttgactactaccgccttgtacatggccattattgtccaatccatctacgctactttgaacccatacgctggtaacttgattaaaggtcgtttcggttacttattagcttctttgggtaagattttcttctctatttctgttacttcttgtatgtgtatcttcgttggtaagttggtctttgctattcaccaaagaagaactttgggtattaagcaattcgacggtttgcaaattttggtcattatgtctactcaatccatgatcatcccaactattatcgtcttgatgtcttttttgagacgtaacgctggttctgtttacaccatggctaccttgttggtcgctttgtccttgccattgtcctccttgtgggctgaagccaagactaccagagactctgcttcttacaccgcttacagaccatctggttctccaaacaaccgttctttgttcgccatcttctctgatagattggcttgtggttctggtagaaacaacagacacgatgatgattctagaggtaacggttctgttaacgccagaaaggctgacgtcgaatctactatcgaaatgtcctcttgttacactgattccccaacctactccaagttcgaagctggtttggacgctagaggtatcgtcttctacaacgaacacggtttgccagttgtctccggtgaagttggtggttcttcctccaacggtactaagttgggttctggtcataagtacgaagtcaacactactgttgttttgtctgatgttgactctccatctccaaccgacgtcacccgtaagtag

SEQ ID NO: 175 - DNA, 1416 bp, Torulaspora delbrueckii
  misc_feature 1..1416: note = Torulaspora delbrueckii (Td)
  source 1..1416: mol_type = genomic DNA
atgtctgactccgcccaaaacttgtccgatttggccttcaactcttcttataacccattggactcctttattacctttacctctatctacggtgataacactgctgttaagttctccgttttacaagacatggttgacgttaatactaatgaagccatcgtttacggtacccgttgtggtgcttctgtcttgacccaaattatcatgtggatgatttctaaaaacagaagaaccccagtctttattattaaccaagtttctttgactttgattttaattcactctgccttgtacttcaagtacttgttgtctggtttcggttccgttgtctacggtttgactgctttcccacaattgattaagccaggtgatttgagagctttcgctgctgctaacatcgttatggtcttgttggtcgcttctattgaagcttccttaatcttccaagtcaaagttatcttcaccggtgataacatgaagagagtcggtttaatcttgactattatttgtacttgtatgggtttagctactgttaccatgtactttattactgccgtcaagtctattgtctctttgtaccgtgacatgtctggttcctccaccgttttatataacgtttctttaattatgttggcttcctccatccactttatggctttgatcttggttgtcaaattgttcttggctgttagatctagaagattcttgggtttgaaacaattcgattctttccacattttgttgatcatctcttgtcaaactttgttggttccatctttattattcattattgcttactcttttccatcttctaagaacattgaatctttgaaggctatcgctgttttgaccgtcgttttgtctttgccattgtcttctatgtgggctactgctgctaataacttcactaactcttcctcctccggttccgactccgctccaaccaatggtggtttctacggtagaggttcttccaacttgtatcctgaaaagactgataacagatccccaaagggtgccagaaacgctttatacgaattaagatctaagaacaatgctgagggtcaagctgatatttacaccgttaccgatattgaaaacgatattttcaacgatttgtccaagccagttgagcaaaacattttctctgatgttcaaattattgattctcattctttgcataaggcttgttctaaagaagacccagtcatgactttgtacactccaaacactgctattgaaggtgaggagagaaaattgtggacttctgactgttcctgttccactaacggttccaccccagttaagaagaagtccaccggtgaatacgccaatttaccaccacacttattaagatatgatgaaaactacgatgaagaagctggtggtagacgtaaggcctccttgaaatggtag

SEQ ID NO: 176 - DNA, 1701 bp, Neurospora crassa
  misc_feature 1..1701: note = Neurospora crassa (Nc)
  source 1..1701: mol_type = genomic DNA
atggcgtcctcttcctcaccacctgcagacattttctcagggatcacgcaatcactaaatagtacacacgcgacgcttacactaccgattccgccagcggacagggatcatctggaaaatcaagtattatttttgtttgacaatcacggtcagttacttaatgtaactacaacttacattgacgcttttaacaatatgctggtctctactactataaactatgcaacgcaaattggagctacttttataatgctagccattatgttattaatgactcccagaaggaggttcaaacgtttaccaacaattattagcttgttagccttatgtattaatttgatcagggtggttttgctggccctgttttttccttctcactggacagacttctacgtgttgtattccggtgactggcagtttgtacctccaggggatatgcaaatatctgttgctgctacggttttgtctatcccagtgacggcattattattgagcgcattgatggttcaagcctggtcaatgatgcaattatggacaccactgtggagggcactagtggtactagtgtccgggctattgtcactggtaactgtggcaatgagtttcgcgaattgcattttccaagcgaaaaatattttgtatgccgaccctttaccctcctactgggtcagaaaattgtacttagcattaacgactgggtctataagttggttcacattcctttttatgataagattggttatgcatatgtggacaaacagatctatattaccaagcatgaagggtttgaaggctatggatgtattgattattacgaattctatattgatgttaatcccagtgttgtttgcaggcttggaatttctggatagtgcctctggatttgagtccgggtctttgactcaaacctctgtagtgattgtcctgcctttgggtactttagtagcacaaagaatagctacgaggggttacatgcccgatagtctggaggcttctagcggaccaaatggttcattgccgttatctaatttaagtttcgctggagggggcggtggtggttctgggggacataaagataaagaaaacggtggcggtattataccgcctactacgaacaatactgctgctactaatttttcttcatcaatcgcgtgttctggtatatcttgtttaccaaaagtcaaaagaatgaccgcgagttcagcctcaagtagccagagaccgttgttgacaatgactaactcaaccatagcgagtaatgacagttcaggtttcccttctcctggcatacataataccactactacgacaacacaataccaatattccatgggaatgaacatgccgaactttcctccagtcccgttcccaggttaccagtcacgtactaccggtgttacttcccatattgtgtccgacggtagacatcaccagggtatgaacaggcacccatctgttgaccattttgatagggaacttgctaggattgatgatgaagatgacgatggttaccctttcgcatcaagtgaaaaggccgttatgcacggagacgatgacgacgatgtggaaaggggacgtcgtagagctctaccaccatccttaggtggagttagagttgaaaggacgatcgagaccaggagcgaggaacgtatgccatctccggacccattgggtgttacgaagcctagatcattcgagtag

SEQ ID NO: 177 - DNA, 1296 bp, Saccharomyces cerevisiae
  misc_feature 1..1296: note = Saccharomyces cerevisiae (Sc)
  source 1..1296: mol_type = genomic DNA
atgtctgatgcggctccttcattgagcaatctattttatgatccaacgtataatcctggtcaaagcaccattaactacacttccatatatgggaatggatctaccatcactttcgatgagttgcaaggtttagttaacagtactgttactcaggccattatgtttggtgtcagatgtggtgcagctgctttgactttgattgtcatgtggatgacatcgagaagcagaaaaacgccgattttcattatcaaccaagtttcattgtttttaatcattttgcattctgcactctattttaaatatttactgtctaattactcttcagtgacttacgctctcaccggatttcctcagttcatcagtagaggtgacgttcatgtttatggtgctacaaatataattcaagtccttcttgtggcttctattgagacttcactggtgtttcagataaaagttattttcacaggcgacaacttcaaaaggataggtttgatgctgacgtcgatatctttcactttagggattgctacagttaccatgtattttgtaagcgctgttaaaggtatgattgtgacttataatgatgttagtgccacccaagataaatacttcaatgcatccacaattttacttgcatcctcaataaactttatgtcatttgtcctggtagttaaattgattttagctattagatcaagaagattccttggtctcaagcagttcgatagtttccatattttactcataatgtcatgtcaatctttgttggttccatcgataatattcatcctcgcatacagtttgaaaccaaaccagggaacagatgtcttgactactgttgcaacattacttgctgtattgtctttaccattatcatcaatgtgggccacggctgctaataatgcatccaaaacaaacacaattacttcagactttacaacatccacagataggttttatccaggcacgctgtctagctttcaaactgatagtatcaacaacgatgctaaaagcagtctcagaagtagattatatgacctatatcctagaaggaaggaaacaacatcggataaacattcggaaagaacttttgtttctgagactgcagatgatatagagaaaaatcagttttatcagttgcccacacctacgagttcaaaaaatactaggataggaccgtttgctgatgcaagttacaaagagggagaagttgaacccgtcgacatgtacactcccgatacggcagctgatgaggaagccagaaagttctggactgaagataataataatttatag

SEQ ID NO: 178 - DNA, 1047 bp, Schizosaccharomyces octosporus
  misc_feature 1..1047: note = Schizosaccharomyces octosporus (So)
  source 1..1047: mol_type = genomic DNA
atgcgtgaaccatggtggaagaactactacaccatgaacggtacccaagtccaaaaccaatccatcccaattttgtccacccaaggttacattcaagttccattgtccaccatcgataaggctgaaagaaacagaattttgactggtatgaccgtttctgctcaattggccttgggtgtcttgatcatggtcatgtctattttgttgtcctccccagaaaagagaaagaccccagttttcatcgtcaactctgcctctatcatttccatgtgtattagagctatcttgatgattgtcaacttgtgttctgaatcctactctttggctgttatgtacggtttcgtcttcgaattggttggtcaatacgttcacgtttttgacattttggttatgattattggtaccatcatcattattaccgctgaagtttccatgttgttgcaagtcagaattatttgtgctcacgacagaaagactcaaagaattgttacctgtatctcttctggtttatccttgatcgtcgttgccttctggttcactgatatgtgtcaagaaattaagtacttgttgtggttgaccccatacaacaaccaccaaatctctggttactactgggtttacttcgtcggtaagatcttgttcgccgtttccattatgttccactctgccgtcttctcctacaagttgttccacgctatccaaattagaaagaagattggtcaattcccattcggtccaatgcaatgtattttaattatttcctgtcaatgtttgttcgttccagctattttcactatcatcgactctttcatccacacttacgacggtttttcctccatgacccaatgtttgttgatcgtctctttgccattgtcctccttgtgggcctcttccactgctttaaagttgcaatctttgaagtctaccacctctccaggtgacactactcaagtttccattagagtcgacagaacctacgacatcaagagaatcccaactgaagaattgtcttctgttgacgaaaccgaaatcaagaagtggccatag

SEQ ID NO: 179 - DNA, 1410 bp, Candida albicans
  misc_feature 1..1410: note = Candida albicans (Ca)
  source 1..1410: mol_type = genomic DNA
atgaatatcaattcaactttcatacctgataaaccaggcgatataattattagttattcaattccaggattagatcaaccaattcaaattcctttccattcattagattcatttcaaaccgatcaagctaaaatagctttagtcatggggataactattgggagttgttcaatgacattaatttttttgatttctataatgtataaaactaataaattaacaaatttaaaattaaaattaaaattaaaatatatcttgcaatggataaatcaaaaaatcttcaccaaaaaaaggaatgacaacaaacaacaacaacaacaacaacaacaacaaattgaatcatcatcatataacaatactactactacgctggggggttataaattatttttattttatcttaattcattgattttattaattggtattattcgatcaggttgttatttaaattataatttaggtccattaaattcacttagttttgtatttactggttggtatgatggatcatcatttatatcatccgatgtaactaatggatttaaatgtattttatatgctttagtggaaatttcattaggtttccaagtttatgtgatgttcaaaacttcaaatttaaaaatttgggggataatggcatcattattatcaattggtttaggattgattgttgttgcctttcaaatcaatttaacaattttatctcatattcgattttcccgggctatatcaactaacagaagtgaagaagaatcatcatcatcattatcatctgattcggttgggtatgtgattaattcaatatggatggatttaccaacaatattattttccattagtattaatataatgacaatattattgattggtaaacttataattgctattagaacaagacgttatttaggattgaaacaatttgatagtttccatattttattaattggtttcagtcaaacattaattattccttcaattattttggtggttcattatttttatttatcacaaaataaagattctttattacaacaaattagtcttttattgattattttaatgttaccattaagttctttatgggctcaaactgctaataatactcataatattaattcatctccaagtttatcattcatatctcgtcatcatctgtctgatagtagtcgtagtggtggttccaatacaattgttagtaatggtggtagtaatggtggtggtggtggtggtgggaatttccctgtttcaggtattgatgcacaattaccacctgatattgaaaaaatcttacatgaagataataattataaattacttaatagtaataatgaaagtgtaaatgatggagatattatcattaatgatgaaggtatgattactaaacaaatcaccatcaaaagagtgtag

SEQ ID NO: 180 - DNA, 1047 bp, Schizosaccharomyces pombe
  misc_feature 1..1047: note = Schizosaccharomyces pombe (Sp)
  source 1..1047: mol_type = genomic DNA
atgagacaaccatggtggaaagactttactattcccgatgcatccgcaattattcaccaaaatattaccattgtctctattgtaggagagattgaagtgccagtttcaacaattgatgcatatgaaagagatagacttttaactggaatgactttgtctgcccaacttgctttaggagtccttaccattttgatggtttgtctattgtcatcatccgaaaaacgaaaacacccagtttttgtttttaattcggcaagtattgttgcaatgtgtcttcgggccattttgaatatagtgaccatatgcagcaatagctacagtatcctggttaattacgggtttatcttaaacatggttcatatgtatgtccatgtgtttaatattttaattttgttgcttgcaccggtcatcatttttactgctgagatgagcatgatgattcaagttcgtataatttgtgcacatgatagaaagacacaaaggataatgactgttattagtgcctgcttaactgttttggttctcgcattttggattactaacatgtgtcaacagattcagtatctgttatggttaactccacttagcagcaagaccattgttggatactcttggccctactttattgctaaaatactttttgcttttagcattatttttcacagtggtgttttttcatacaaactctttcgtgccatattaatacggaaaaaaattgggcaatttccatttggtccgatgcagtgtattttagttattagctgccaatgtcttattgttccagctacctttactataatagatagttttatccatacgtatgatggctttagctctatgactcaatgtctgctaatcatttctcttcctctttcgagtttatgggcgtctagtacagctctgaaattgcaaagcatgaaaacttcatctgcgcaaggagaaaccaccgaggtttcgattagagttgatagaacgtttgatatcaaacatactcccagtgacgattattcgatttctgatgaatctgaaactaaaaagtggacgtag

SEQ ID NO: 181 - DNA, 1422 bp, unidentified
  misc_feature 1..1422: note = Vanderwaltozyma polyspora 2 (Vp2)
  source 1..1422: mol_type = genomic DNA
atgtcaggaattgatgatatgggtgataaaccagatattttaggtttattttatgatgctaactatgatccaggtcaaggtatactcacatttatttcaatgtacgggaatactactataacttttgatgagttacagttagaggtcaatagtttaattacaagtggtattatgttcggcgtcagatgtggtgctgcttgtttgacattgttaataatgtggatgatttctaagaataagaagactccaatttttattattaatcaatgctcgctaatccttattattatgcattcaggtttatattttaagaatattctatcaaatttgaattctttatcatatatcttaactgggtttactcaaaatatcactaaaaataatatacatgtctttggtgccgctaatattattcaagttttattagtagcaaccattgaactgtcgttagtgtttcaaattcgagtcatgtttaaaggtgacagttttagaaaagctggttacggtttgttgtcaattgcgtctggtttgggtatagctactgtcgtcatgtatttttactctgccattacaaatatgattgctgtttataatcaaacttacaactccactgctaaattatttaacgttgcaaacattcttctgtctacatcgataaattttatgacggtagtattaattgttaaattatttttggctgttagatcaagaagatatttgggtttaaagcagttcgatagtttccatattttattgattatgtcatgtcaaacattgattgtaccatcaattctttttatcttatcatacgctttaagtactaagctgtacactgatcatttagttgtcattgcaactttattagtcgttctatctttaccattatcttcgatgtgggcaagcgctgcaaataattctcctaaaccaagctcgtttacaaccgattattcaaacaagaatcctagtgacacaccaagcttctacagtcaaagtattagttcctcgatgaaaagcaaattcccaagcaaattcatacccttcaatttcaagtctaaagacaattcttctgacactagatcagaaaatacatatattggcaattatgacatggaaaagaatggatcaccaaatcactcttattcttccaaagatcaaagtgaagtttacactataggtgtaagctctatgcacacagatataaagtcacaaaagaatatcagtggacagcatttatataccccaagtacagagattgatgaagaagctagagacttctgggcgggcagagctgttaataattcagttccaaatgactatcaaccatctgagttaccagcatcgattcttgaagaattgaattcactggatgaaaataatgaaggtttcttggagacaaaaagaataacatttagaaaacaatag

SEQ ID NO: 182 - DNA, 966 bp, Scheffersomyces stipitis
  misc_feature 1..966: note = Scheffersomyces stipitis (Ss)
  source 1..966: mol_type = genomic DNA
atggatactagtatcaatactctcaaccctgcgaatatcattgtcaactacaccttgccaaatgatcctagagtaattagtgtcccatttggagcttttgacgaatatgttaaccaatctatgcaaaaggccattatccatggagtttccattggttcatgcaccataatgcttttaattattttgatcttcaatgtcaaacgcaagaagtcgccagctttctatcttaattcggttacgttgactgcaatgattattcggtctgctcttaatttggcatatttgctaggtcctttggctggattaagttttacgttctccggcttggtaactccagaaaccaatttctctgtctctgaagccaccaatgctttccaggttattgttgttgctcttatcgaggcgtccatgacatttcaggtgttcgtcgtcttccaatcaccagaagtgaagaagttgggtatagctcttacctccatatctgcattcacgggtgctgctgctgtaggatttactatcaatagtacaatccaacaatcgagaatttatcattcagttgtcaatggaactcctacgccaacggtcgctacctggtcttgggttagagatgtgcctacgatacttttttctacttcggttaacataatgtctttcatcttgattctcaagttagggtttgccataaagacaagaagataccttggccttcggcaatttggcagtttgcacatcttattgatgatggctactcaaacattattggccccatctattctcattcttgtacattacggatatggcacatctctgaatagccagctcattcttataagttacttgcttgttgttttgtctttaccagtatcctctatctgggcagcaacagccaacaattctcctcaacttccatcttccgcaactctttcattcatgaacaaaacgacctctcacttttctgaaagctag

SEQ ID NO: 183 - DNA, 1254 bp, Candida glabrata
  misc_feature 1..1254: note = Candida glabrata (Cg)
  source 1..1254: mol_type = genomic DNA
atggagatgggctacgatccaagaatgtataatccaagaaatgaatacttgaatttcacgtcggtatatgatgtaaatgacacaatcagattttcgactctggacgccattgtaaaaggattgcttagaattgccattgttcatggagttagattgggagcaatattcatgacgttaataataatgtttatctcatcaaatacatggaaaaaacccatatttataattaacatggtgtcgttgatgttagttatgattcattccgcacttagcttccattaccttttatcgaattattcttcaatttcttatatactgacagggtttcctcagttgattacaagcaataataaacgaattcaagatgcagcgagtatagtccaagttttattggttgctgcgatagaagcatcattggtatttcagattcatgttatgtttacgattgaaaacattaagcttattagagaaatagtactctctatatcgatagcaatgggattggcaacagtggctacatatcttgctgcagcaataaagctgataagaggactgcatgatgaggtaatgccacaaacacatcttattttcaatttatctataatattgcttgcatcctccataaattttatgacatttatattggtcattaaacttttcttcgctattagatctagaagatatctcggtcttcgtcaattcgatgcttttcatattttattaatcatgttctgccagtcattattgataccctcagtattatatattatagtttacgcggttgatagcagatctaatcaggattatctgattccaattgccaatttatttgttgttttatctttgccattatcctctatctgggctaacacatcaaataactcatccagatctccaaaatattggaaaaactctcaaacgaataagagcaatgggtcttttgtctcttcaatatctgtcaatagtgactcacaaaaccctttgtacaaaaagattgtacgttttacatcaaaaggcgacactacccgtagtattgtaagtgattcaacattagcagaggtgggaaaatactctatgcaagacgttagcaattcaaactttgaatgtcgagaccttgattttgagaaggtaaaacatacttgcgaaaattttggcagaatatctgaaacatatagtgagttaagtactttagataccactgccctcaatgagactcggttgttttggaaacaacaaagtcagtgtgacaaatag

SEQ ID NO: 184 - DNA, 1155 bp, unidentified
  misc_feature 1..1155: note = Vanderwaltozyma polyspora 1 (Vp1)
  source 1..1155: mol_type = genomic DNA
atgagttcccaatcacacccaccgctaatcgatttattttacgattccagttatgaccctggtgaaagtttaatttattacacatccatctatggtaataatacatacataacttttgatgaactccagacgatagtgaacaagaaggtcacacaaggtatcttatttggtgtcagatgtggtgctgctttcctgatgttggtagcaatgtggttgatttccaaaaataaaagatctagaattttcattaccaaccaatgttgtctggtcttcatgataatgcattctggtctttattttaggtacctgctttcaaggtacggttcagttactttcattctaacagggttccaacaactgcttacaagaaatgacattcatatttatggagctactgattttatccaagtagctttggtagcttgcatagaattatctcttattttccaaataaaagtgatattcgctggtacaaactatggtaagttggctaattatttcatcactctaggttcattattgggtttagccacctttggtatgtacatgcttactgctattaacggtacaataaaattatacaataacgaatatgacccaaaccaaaggaaatactttaacatttctacaatattgcttgcatcatcaattaatatgctaacgctgatacttatattgaagctggtggcagcaattagaacaagacgttacttaggtttgaagcaattcgatagttttcacatcctattaatcatgtcgactcaaacattaataattccttctatcttatttattctatcatacagtttgagagaggatatgcatactgatcaattaataatcatcggaaatctgatcgtggtattgtcattaccattgtcctcaatgtgggcttcgtctctaaacaattcaagtaaacctacatctttgaatactgatttctcagggccaaaatcaagtgaagaagggacagcaataagtttgctatcacaaaacatggaaccatcaatagtcactaaatatacaagaagatcacctgggttatacccagtaagcgtgggtacaccaattgaaaaagaagcatcatacactctttttgaagctactgacattgattttgaaagcagtagtaacgatatcacaaggacttcatag

SEQ ID NO: 185 - DNA, 1359 bp, Zygosaccharomyces bailii
  misc_feature 1..1359: note = Zygosaccharomyces bailii (Zb)
  source 1..1359: mol_type = genomic DNA
atgtctggtttggctaacaacacctcttacaacccattggaatctttcattattttcacttctgtttacggtggtgataccatggttaagttcgaagacttgcaattagtcttcaccaagcgtattactgaaggtattttgttcggtgtcaaggttggtgccgcttctttgactatgattgttatgtggatgatttccagaagaagaacctccccaatcttcatcatgaaccaattgtctttggttttcaccatcttgcacgcttctttttactttaagtacttattggacggtttcggttctattgtctacactttgaccttgttcccacaattaattacttcctctgacttgcacgttttcgctactgctaacgttgttgaagtcttattggtttcttccatcgaagcctctttggttttccaagtcaacgtcatgttcgctggttctaaccacagaaagttcgcttggttgttggtcggtttctctttgggtttggctttggccactgtcgctttgtacttcgttactgctgtcaagatgatcgcttccgcttacgcttctcaaccaccaactaacccaatctacttcaacgtttccttgttcttgttggctgcctccgttttcttgatgactttaatgttgaccgtcaagttgatcttggctatcagatccagaagattcttgggtttgaagcaattcgactctttccacattttgttgattatgtcttgtcaaactttgatcgctccatctgttttgtacatcttgggttttattttggatcacagaaagggtaacgactacttgattaccgtcgctcaattgttggtcgttttgtctttgccattgtcctccatgtgggccactactgctaacgatgcttcctccggtacttctatgtcttccaaggaatccgtctacggttctgattccttatactctaagtctaagtgttcccaattcaccagaaccttcatgaacagattctctactaagccaactaagaacgacgaaatttctgattccgctttcgtcgctgttgattccttggaaaagaacgctccacaaggtatctctgaacacgtttgtgaattcccacaatctgacttatctgatcaagctacttccatctcctccagaaaaaaggaagctgttgtttacgcttccactgttgatgaagataagggttctttctcctctgacatcaacggttacactgttaccaacatgccattggcttccgctgcttctgctaactgtgaaaactccccatgtcacgttccaagaccatacgaagaaaacgaaggtgtcgtcgaaaccagaaaaattattttgaagaagaacgtcaaatggtag

SEQ ID NO: 186 - protein, 431 aa, Saccharomyces cerevisiae
  SITE 1..431: note = Saccharomyces cerevisiae (ScSte2)
MSDAAPSLSNLFYDPTYNPGQSTINYTSIYGNGSTITFDELQGLVNSTVTQAIMFGVRCGAAALTLIVMWMTSRSRKTPIFIINQVSLFLIILHSALYFKYLLSNYSSVTYALTGFPQFISRGDVHVYGATNIIQVLLVASIETSLVFQIKVIFTGDNFKRIGLMLTSISFTLGIATVTMYFVSAVKGMIVTYNDVSATQDKYFNASTILLASSINFMSFVLVVKLILAIRSRRFLGLKQFDSFHILLIMSCQSLLVPSIIFILAYSLKPNQGTDVLTTVATLLAVLSLPLSSMWATAANNASKTNTITSDFTTSTDRFYPGTLSSFQTDSINNDAKSSLRSRLYDLYPRRKETTSDKHSERTFVSETADDIEKNQFYQLPTPTSSKNTRIGPFADASYKEGEVEPVDMYTPDTAADEEARKFWTEDNNNL

SEQ ID NO: 187 - protein, 469 aa, Candida albicans
  SITE 1..469: note = Candida albicans (CaSte2)
MNINSTFIPDKPGDIIISYSIPGLDQPIQIPFHSLDSFQTDQAKIALVMGITIGSCSMTLIFLISIMYKTNKLTNLKLKLKLKYILQWINQKIFTKKRNDNKQQQQQQQQQIESSSYNNTTTTLGGYKLFLFYLNSLILLIGIIRSGCYLNYNLGPLNSLSFVFTGWYDGSSFISSDVTNGFKCILYALVEISLGFQVYVMFKTSNLKIWGIMASLLSIGLGLIVVAFQINLTILSHIRFSRAISTNRSEEESSSSLSSDSVGYVINSIWMDLPTILFSISINIMTILLIGKLIIAIRTRRYLGLKQFDSFHILLIGFSQTLIIPSIILVVHYFYLSQNKDSLLQQISLLLIILMLPLSSLWAQTANNTHNINSSPSLSFISRHHLSDSSRSGGSNTIVSNGGSNGGGGGGGNFPVSGIDAQLPPDIEKILHEDNNYKLLNSNNESVNDGDIIINDEGMITKQITIKRV

SEQ ID NO: 188 - protein, 348 aa, Schizosaccharomyces pombe
  SITE 1..348: note = Schizosaccharomyces pombe (SpSte2)
MRQPWWKDFTIPDASAIIHQNITIVSIVGEIEVPVSTIDAYERDRLLTGMTLSAQLALGVLTILMVCLLSSSEKRKHPVFVFNSASIVAMCLRAILNIVTICSNSYSILVNYGFILNMVHMYVHVFNILILLLAPVIIFTAEMSMMIQVRIICAHDRKTQRIMTVISACLTVLVLAFWITNMCQQIQYLLWLTPLSSKTIVGYSWPYFIAKILFAFSIIFHSGVFSYKLFRAILIRKKIGQFPFGPMQCILVISCQCLIVPATFTIIDSFIHTYDGFSSMTQCLLIISLPLSSLWASSTALKLQSMKTSSAQGETTEVSIRVDRTFDIKHTPSDDYSISDESETKKWT

SEQ ID NO: 189 - protein, 348 aa, Schizosaccharomyces octosporus
  SITE 1..348: note = Schizosaccharomyces octosporus (SoSte2)
MREPWWKNYYTMNGTQVQNQSIPILSTQGYIQVPLSTIDKAERNRILTGMTVSAQLALGVLIMVMSILLSSPEKRKTPVFIVNSASIISMCIRAILMIVNLCSESYSLAVMYGFVFELVGQYVHVFDILVMIIGTIIIITAEVSMLLQVRIICAHDRKTQRIVTCISSGLSLIVVAFWFTDMCQEIKYLLWLTPYNNHQISGYYWVYFVGKILFAVSIMFHSAVFSYKLFHAIQIRKKIGQFPFGPMQCILIISCQCLFVPAIFTIIDSFIHTYDGFSSMTQCLLIVSLPLSSLWASSTALKLQSLKSTTSPGDTTQVSIRVDRTYDIKRIPTEELSSVDETEIKKWP

SEQ ID NO: 190 - protein, 346 aa, Schizosaccharomyces japonicus
  SITE 1..346: note = Schizosaccharomyces japonicus (SjSte2)
MYSWDEFRSPKQAEVLNQTVTLETIVSTIQLPISEIDSMERNRLLTGMTVAVQVGLGSFILVLMCIFSSSEKRKKPVFIFNFAGNLVMTLRAIFEVIVLASNNYSIAVQYGFAFAAVRQYVHAFNIIILLLGPFILFIAEMSLMLQVRIICSQHRPTMITTTVISCIFTVVTLAFWITDMSQEIAYQLFLKNYNMKQIVGYSWLYFIAKITFAASIIFHSSVFSFKLMRAIYIRRKIGQFPFGPMQCIFIVSCQCLIVPAIFTLIDSFTHTYDGFSSMTQCLLIISLPLSSLWATHTAQKLQTMKDNTNPPSGTQLTIRVDRTFDMKFVSDSSDGSFTEKTEETLP

SEQ ID NO: 191 - protein, 347 aa, Baudoinia compniacensis; identical to SEQ ID NO: 151
  SITE 1..347: note = Baudoinia compniacensis (BcomSte2)

SEQ ID NO: 192 - protein, 360 aa, Candida lusitaniae
  SITE 1..360: note = Candida (Clavispora) lusitaniae (ClSte2)
MNPADINIEYTLGDTAFSSTFADFEAWKTRNTQFAIVNGVALACGIILMVVSWIIIVNKRAPIFAMNQTMLVIMVIKSAMYLKHIMGPLNSLTFRFTGLMEESWAPYNVYVTINVLHVLLVAAVESSLVFQIHVVFKSSRARVAGRAIVSAMSTLALLIVSLYLYSTVRHAQTLRAELSHGDTTTVEPWVDNVPLILFSASLNVLCLLLALKLVFAVRTRRHLGLRQFDSFHILIIMATQTFVIPSSLVIANYRYASSPLLSSISIIVAVCNLPLCSLWACSNNNSSYPTSSQNTILSRYETETSQATDASSTTCAGIAEKGFDKSPDSPTFGDQDSVSISHILDSLEKDVEGVTTHRLT

SEQ ID NO: 193 - protein, 412 aa, Candida tropicalis
  SITE 1..412: note = Candida tropicalis (CtSte2)
MDINNTIQSSGDIIITYTIPGIEEPFELPFEVLNHFQSEQSKNCLVMGVMIGSCSVLLIFLVGILFKTNKFSTIGKSKNLSKNFLFYLNCLITFIGIIRAACFSNYLLGPLNSASFAFTGWYNGESYASSEAANGFRVILFALIETSMVFQVFVMFRGAGMKKLAYSVTILCTALALVVVGFQINSAVLSHRRFVNTVNEIGDTGLSSIWLDLPTILFSVSVNLMSVLLIGKLIMAIKTRRYLGLKQFDSFHVLLICSTQTLLVPSLILFVHYFLFFRNANVMLINISILLIVLMLPFSSLWAQTANTTQYINSSPSFSFISREPSANSTLHSSSGHYSEKSYGINKLNTQGSSPATLKDDHNSVILEATNPMSGFDAQLPPDIARFLQDDIRIEPSSTQDFVSTEVTYKKV

SEQ ID NO: 194 - protein, 477 aa, Saccharomyces castellii
  SITE 1..477: note = Saccharomyces castellii (ScaSte2)
MSDAPPPLSELFYNSSYNPGLSIISYTSIYGNGTEVTFNELQSIVNKKITEAIMFGVRCGAAILTIIVMWMISKKKKTPIFIINQVSLFLILLHSAFNFRYLLSNYSSVTFALTGFPQFIHRNDVHVYAAASIFQVLLVASIEISLMFQIRVIFKGDNFKRIGTILTALSSSLGLATVAMYFVTAIKGIIATYKDVNDTQQKYFNVATILLASSINFMTLILVIKLILAIRSRRFLGLKQFDSFHILLIMSFQSLLAPSILFILAYSLDPNQGTDVLVTVATLLVVLSLPLSSMWATAANNASRPSSVGSDWTPSNSDYYSNGPSSVKTESVKSDEKVSLRSRIYNLYPKSKSEFEQSSEHTYVDKVDLENNFYELSTPITERSPSSIIKKGKQGISTRETVKKLDSLDDIYTPNTAADEEARKFWSEDVSNELDSLQKIETETSDELSPEMLQLMIGQEEEDDNLLATKKITVKKQ

SEQ ID NO: 195 - protein, 376 aa, Candida auris
  SITE 1..376: note = Candida auris (CauSte2)
MEFTGDIVLKYTLGGEEYLSTFEQLDSSVNRSLELGVVHGIAIACGVLLMVLAWVIIIKKKNPIFVLNQLTLLLMVIKSSLYLAFLFGPLSSLTYKFTRVLPHDKWHAFHVYIATNVIHTLLIATVEMTLVFQIYIIFKSPEVRHLGYILTGAASALALTIVALYIHSTVISAVQLKEQLLMHEIKITNSWVNNVPIILFSASLNVVCIILIAKLALAIKTRRYLGLKQFDGLHILMITSTQTFIVPSVLMIVNYKQSSSYLTLLANISVILVVCNLPLSSLWAASANNSSTPTSSANTVFSRWDSKFSDTETIAHELPLIPGKAEKLQLVSPITEKGDTHTMCESHGDQDLIDKMLDDIEGAVMTTEFNLNNRTV

SEQ ID NO: 196 - protein, 403 aa, Candida parapsilosis; identical to SEQ ID NO: 150
  SITE 1..403: note = Candida parapsilosis (CpSte2)

SEQ ID NO: 197 - protein, 436 aa, Kluyveromyces lactis
  SITE 1..436: note = Kluyveromyces lactis (KlSte2)
MSEEIPSLNPLFYNETYNPLQSVLTYSSIYGDGTEITFQQLQNLVHENITQAIIFGTRIGAAGLALIIMWMVSKNRKTPIFIINQSSLVLTIVQSALYLSYLLSNFGGVPFALTLFPQMIGDRDKHLYGAVTLIQCLLVACIEVSLVFQVRVIFKADRYRKIGIILTGVSASFGAATVAMWMITAIKSIIVVYDSPLNKVDTYYYNIAVILLACSINFITLLLSVKLFLAFRARRHLGLKQFDSFHILLIMSTQTLIGPSVLYILAYALNNKGVKSLTSIATLLVVLSLPLTSIWAAAANDAPSASTFYRQFNPYSAQNRDDSSSYSYGKAFSDKYSFSNSPQTSDGCSSKELELSTQLEMDLESGESFMDRAKRSDFVSSPGSTDATVIKQLKASNIYTSETDADEEARAFWVNAIHENKDDGLMQSKTVFKELR

SEQ ID NO: 198 - protein, 386 aa, Fusarium graminearum
  SITE 1..386: note = Fusarium graminearum (FgSte2)
MSKEVFDPFTQNVTFFAPDGKTEISIPVAAIDQVRRMMVNTTINYATQLGACLIMLVVLLVMVPKEKFRRPFMILQITSLVISCCRMLLLSIFHSSQFLDFYVFWGDDHSRIPRSAYAPSVAGNTMSLCLVISVETMLMSQAWTMVRLWPNVWKYIIAGVSLIVSIMAISVRLAYTIIQNNAVLKLEPAFHMFWLIKWTVIMNVASISWWCAIFNIKLVWHLISNRGILPSYKTFTPMEVLIMTNGILMIIPVIFASLEWAHFVNFESASLTLTSVAVILPLGTLAAQRIASSAPSSANSTGASSGIRYGVSGPSSFTGFKAPSFSTGTTDRPHVSIYARCEAGTSSREHINPQGVELAKLDPETDHHVRVDRAFLQREERIRAPL

SEQ ID NO: 199 - protein, 354 aa, Komagataella pastoris
  SITE 1..354: note = Komagataella (Pichia) pastoris (KpSte2)
MEEYSDSFDPSQQLLNFTSLYGETDATFAELDDYHFYVVKYAIVYGARIGVGMFCTLMLFVVSKSWKTPIFVLNQSSLILLIIHSGFYIHYLTNQFSSLTYMFTRIPNETHAGVDLRINVVTNTLYALLILSIEISLIYQVFVIFKGVYENSLRWIVTIFTALFAAAVVAINFYVTTLQSVSMYNSNVDFPRWASNVPLILFASSVNWACLLLSLKLFFAIKVRRSLGLRQFDTFHILAIMFSQTLIIPSILIVLGYTGTRDRDSLASLGFLLIVVSLPFSSMWAATANNSNIPTSTGSFAWKNRYSPSTYSDDTTAVSKSFTIMTAKDECFTTDTEGSPRFIKGDRTSEDLHF

SEQ ID NO: 200 - protein, 419 aa, Candida tenuis
  SITE 1..419: note = Candida tenuis (CnSte2)
MDSYLLNHPGDISLNFALPLSDEVYTITFNDLDSQSSFSIQYLVIHSCAITVCLTLLVLLNLFIRNKKTPVFVLNQVILFFAIVRSSLFIGFMKSPLSTITASFTGIISDDQKHFYKVSVAANAALIILVMLIQVSFTYQIYIIFRSPEVRKFGVFMTSALGVLMAVTFGFYVNSAVASTKQYQHIFYSTDPYIMDSWVTGLPPILYSASVIAMSLVLVLKLVAAVRTRRYLGLKQFSSYHILLIMFTQTLFVPTILTILAYAFYGYNDILIHISTTITVVLLPFTSIWASIANNSRSLMSAASLYFSGSNSSLSELSSPSPSDNDTLNENVFAFFPDKLQKMNSSEAVSAVDKVVVHDHFDTISQKSIPHDILEILQGNEGGQMKEHISVYSDDSFSKTTPPIVGGNLLITNTDIGMK

SEQ ID NO: 201 - protein, 398 aa, Botrytis cinerea
  SITE 1..398: note = Botrytis cinerea (BcSte2)
MASNSSNFDPLTQSITILMADGITTVSFTPLDIDFFYYYNVACCINYGAQAGACLLMFFVVVVLTKAVKRKTLLFVLNVLSLIFGFLRAMLYAIYFLQGFNDFYAAFTFDFSRVPRSSYASSVAGSVIPLCMTITVNMSLYLQAYTVCKNLDDIKRIILTTLSAIVALLAIGFRFAATVVNSVAILATSASSVPMQWLVKGTLVTETISIWFFSLIFTGKLVWTLYNRRRNGWRQWSAVRILAAMGGCTMVIPSIFAILEYVTPVSFPEAGSIALTSVALLLPISSLWAGMVTDEETSAIDVSNLTGSRTMLGSQSGNFSRKTHASDITAQSSHLDFSSRKGSNATMMRKGSNAMDQVTTIDCVVEDNQANRGLRDSTEMDLEAMGVRVNKSYGVQKA

SEQ ID NO: 202 - protein, 412 aa, Beauvaria bassiana
  SITE 1..412: note = Beauvaria bassiana (BbSte2)
MDGSSAPSSPTPDPTFDRFAGNVTFFLADHITTTSVPMPVLNAYYDESLCTTMNYGAQLGACLVMLVVVVALTPAAKLARRPASALHLVGLLLCAVRSGLLFAYFVSPISHFYQVWAGDFSAVSRRYWDASLAANTLAFPLVVVVEAALINQAWTMVAFWPRAAKAAACACSAVIVLLTIGTRLAYTIVQNHAIVTAVPPEHFLWAIQWSAVMGAVSIFWFCAVFNVKLVCHLVANRGILPSISVVNPMEVLVMTNGTLMIIPSIFAGLEWAKFTNFESGSLTLTSVIIILPLGTLAAQRISGQGSQGYQAGHLFHEQQQQQARTRSGAFGSASQQSHPTNKVPSSITLSTSGTPITPQISAGSRPELPLVDRSERLDPIDLELGRIDAFRGSSDFSPSTARPKRMQRDNFA

SEQ ID NO: 203 - protein, 296 aa, Hypocrea jecorina
  SITE 1..296: note = Hypocrea jecorina (HjSte2)
MSSFDPYTQNITILVSPSSPPISIPIPVIDAFNDETASIITNYAAQLGAALAMLLVLLAATPTARLLRADGPSLLHALALLVCVVRTVLLIYFFLTPFSHFYQVWTGDFSQVPAWNYRASIAGTVLSTLLTVVTDAALVNQAWTMVSLFAPRTKRAVCVLSLLITLLAISFRVAYTVIQCEGIAELAAPRQYAWLIRATLIFNICSIAWFCALFNSKLVAHLVTNRGVLPSRRAMSPMEVLIMANGILMIVPVVFAILEWHHFINFEAGSLTPTSIAIILPLSSLAAQRIANTSSS

SEQ ID NO: 204 - protein, 397 aa, Pseudogymnoascus destructans
  SITE 1..397: note = Pseudogymnoascus destructans (PdSte2)
MSTANVHLPADFDPTRQNITIYTPDGTPVVATLPMINLFNRQNNEICVVYGCQLGASLIMFLVVLLTTRVSKRKSPIFVLNVLSLIISCLRSLLQILYYIGPWTEIYRYLSFDYSTVPASAYANSVAATLLTLFLLITIEASLVLQTNVVCKSMSSHIRWPVTALSMVVSLLAISFRFGLTIRNIEGILGATVKSDSLMFSGASLISETASIWFFCTIFVIKLGWTLYQRKKMGLKQWGPMQIITIMAGCTMLIPSLFTVLEFFPEETFYEAGTLAICLVAILLPLSSVWAAAAIDGDEPVRPHGSTPKFASFNMGSDYKSSSAHLPRSIRKASVPAEHLSRTSEEELGDDGTLNRGGAYGMDRMSGSISPRGVRIERTYEVHTAGRGGSIEREDIF

SEQ ID NO: 205 - protein, 435 aa, Lodderomyces elongisporous; identical to SEQ ID NO: 153
  SITE 1..435: note = Lodderomyces elongisporous (LeSte2)

SEQ ID NO: 206 - protein, 380 aa, Penicillium chrysogenum
  SITE 1..380: note = Penicillium chrysogenum (PrSte2)
MATSSPIQPFDPFTQNVTFRLQDGTEFPVSVKALDVFVMYNVRVCINYGCQFGASFVLLVILVLLTQSDKRRSAVFILNGLALFLNSSRLLFQVIHFSTAFEQVYPYVSGDYSSVPWSAYAISIVAVVLTTLVVVCIEASLVIQVHVVCSTLRRRYRHPLLAISILVALVPIGFRCAWMVANCKAIIKLTYTNDVWWIESATNICVTISICFFCVIFVTKLGFAIKQRRRLGVREFGPMKVIFVMGCQTMVVPAIFSITQYYVVVPEFSSNVVTLVVISLPLSSIWAGAVLENARRTGSQDRQRRRNLWRALVGGAESLLSPTKDSPTSLSAMTAAQTLCYSDHTMSKGSPTSRDTDAFYGISVEHDISINRVQRNNSIV

SEQ ID NO: 207 - protein, 356 aa, Paracoccidioides brasiliensis
  SITE 1..356: note = Paracoccidioides brasiliensis (PbSte2)
MAPSFDPFNQNVVFHKADGTPFNVSIHELDDFVQYNTRVCINYSSQLGASVIAGLMLAMLTHSEKRRLPVFFLNTFALAMNFARLLCMTIYFTTGFNKSYAYFGQDYSQVPGSAYAASVLGVVFTTLLVISMEMSLLIQTRVVCTTLPDIQRYLLMAVSSAISLMAIGFRLGLMVENCIAIVQASNFAPFIWLQSASNITITISTCFFSAVFVTKLAYALVTRIRLGLTRFGAMQVMFIMSCQTMVIPAIFSILQYPLPKYEMNSNLFTLVAIFLPLSSLWASVATKSSFETSSSGRHQYLWPSEQSNNVTNSEIKYQVSFSQNHTTLRSGGSVATTLSPDRLDPVYSEVEAGTKA

SEQ ID NO: 208 - protein, 395 aa, Candida guilliermondii
  SITE 1..395: note = Candida (Pichia) guilliermondii (CguSte2)
MKSCSIGFGIPFINEPNFETVSILTMDVSFIDADVNPDNILLNFTIPGYQNGFSVPMVVINELQKSQMKYAIVYGCGVGASLILLFVVWILCSRKTPLFIMNNIPLVLYVISSSLNLAYITGPLSSVSVFLTGILTSHDAINVVYASNALQMLLIFSIQSTMAYHVYVMFKSPQIKYLRYMLVGFLGCLQIVTTCLYINYNVLYSRRMHKLYETGQTYQDGTVMTFVPFILFQCSVNFSSIFLVLKLIMAIRTRRYLGLRQFGGFHILMIVSLQTMLVPSILVLVNYAAHKAVPSNLLSSVSMMIIVLSLPASSMWAAAANASSAPSSAASSLFRYTTSDSDRTLETKSDHFIMKHESHNSSPNSSPLTLVQKRISDATLELPKELEDLIDSTSI

SEQ ID NO: 209 - protein, 443 aa, Zygosaccharomyces rouxii
  SITE 1..443: note = Zygosaccharomyces rouxii (ZrSte2)
MSEINNSTYNPMNAYVTFTSIYGDDTMVRFKDVELVVNKRVTEAIMFGVKVGAASLTLIIMWMISKKRTTPIFIINQSSLVFTIIHASLYFGYLLSGFGSIVYNMTSFPQLISSNDVRVYAATNIFEVLLVASIEISLVFQVKVMFANNNGRRWTWCLMVVSIGMALATVGLYFATAVELIRAAYSNDTVSRHVFYNVSLILLASSVNLMTLMLVVKLVLAIRSRRFLGLKQFDSFHILLIMSCQTLIAPSILFILGWTLDPHTGNEVLITVGQLLIVLSLPLSSMWATTANNTSSSSSSVSCNDSSFGNDNLCSKSSQFRRTFMNRFRPKSVNGDGNSENTFVTIDDLEKSVFQELSTPVSGESKIDHDHASSISCQKTCNHVHASTVNSDKGSWSSDGSCGSSPLRKTSTVNSEDLPPHILSAYDDDRGIVESKKIILKKL

SEQ ID NO: 210 - protein, 435 aa, Geotrichum candidum
  SITE 1..435: note = Geotrichum candidum (GcSte2)
MAEDSIFPNNSTSPLTNPIVVETIKGTAYIPLHYLDDLQYEKMLLASLFSVRIATSFVVIIWYFVAVNKAKRSKFLYIVNQVSLLIVFIQSILSLIYVFSNFSKMSTILTGDYTGITKRDINVSCVASVFQFLFIACIELALFIQATVVFQKSVRWLKFSVSLIQGSVALTTTALYMAIIVQSIYATLNPYAGNLIKGRFGYLLASLGKIFFSISVTSCMCIFVGKLVFAIHQRRTLGIKQFDGLQILVIMSTQSMIIPTIIVLMSFLRRNAGSVYTMATLLVALSLPLSSLWAEAKTTRDSASYTAYRPSGSPNNRSLFAIFSDRLACGSGRNNRHDDDSRGNGSVNARKADVESTIEMSSCYTDSPTYSKFEAGLDARGIVFYNEHGLPVVSGEVGGSSSNGTKLGSGHKYEVNTTVVLSDVDSPSPTDVTRK

SEQ ID NO: 211 - protein, 566 aa, Neurospora crassa
  SITE 1..566: note = Neurospora crassa (NcSte2)
MASSSSPPADIFSGITQSLNSTHATLTLPIPPADRDHLENQVLFLFDNHGQLLNVTTTYIDAFNNMLVSTTINYATQIGATFIMLAIMLLMTPRRRFKRLPTIISLLALCINLIRVVLLALFFPSHWTDFYVLYSGDWQFVPPGDMQISVAATVLSIPVTALLLSALMVQAWSMMQLWTPLWRALVVLVSGLLSLVTVAMSFANCIFQAKNILYADPLPSYWVRKLYLALTTGSISWFTFLFMIRLVMHMWTNRSILPSMKGLKAMDVLIITNSILMLIPVLFAGLEFLDSASGFESGSLTQTSVVIVLPLGTLVAQRIATRGYMPDSLEASSGPNGSLPLSNLSFAGGGGGGSGGHKDKENGGGIIPPTTNNTAATNFSSSIACSGISCLPKVKRMTASSASSSQRPLLTMTNSTIASNDSSGFPSPGIHNTTTTTTQYQYSMGMNMPNFPPVPFPGYQSRTTGVTSHIVSDGRHHQGMNRHPSVDHFDRELARIDDEDDDGYPFASSEKAVMHGDDDDDVERGRRRALPPSLGGVRVERTIETRSEERMPSPDPLGVTKPRSFE

SEQ ID NO: 212 - protein, 473 aa, unidentified
  REGION 1..473: note = Vanderwaltozyma polyspora 2 (Vp2Ste2)
MSGIDDMGDKPDILGLFYDANYDPGQGILTFISMYGNTTITFDELQLEVNSLITSGIMFGVRCGAACLTLLIMWMISKNKKTPIFIINQCSLILIIMHSGLYFKNILSNLNSLSYILTGFTQNITKNNIHVFGAANIIQVLLVATIELSLVFQIRVMFKGDSFRKAGYGLLSIASGLGIATVVMYFYSAITNMIAVYNQTYNSTAKLFNVANILLSTSINFMTVVLIVKLFLAVRSRRYLGLKQFDSFHILLIMSCQTLIVPSILFILSYALSTKLYTDHLVVIATLLVVLSLPLSSMWASAANNSPKPSSFTTDYSNKNPSDTPSFYSQSISSSMKSKFPSKFIPFNFKSKDNSSDTRSENTYIGNYDMEKNGSPNHSYSSKDQSEVYTIGVSSMHTDIKSQKNISGQHLYTPSTEIDEEARDFWAGRAVNNSVPNDYQPSELPASILEELNSLDENNEGFLETKRITFRKQ

SEQ ID NO: 213 - protein, 321 aa, Scheffersomyces stipitis
  SITE 1..321: note = Scheffersomyces stipitis (SsSte2)
MDTSINTLNPANIIVNYTLPNDPRVISVPFGAFDEYVNQSMQKAIIHGVSIGSCTIMLLIILIFNVKRKKSPAFYLNSVTLTAMIIRSALNLAYLLGPLAGLSFTFSGLVTPETNFSVSEATNAFQVIVVALIEASMTFQVFVVFQSPEVKKLGIALTSISAFTGAAAVGFTINSTIQQSRIYHSVVNGTPTPTVATWSWVRDVPTILFSTSVNIMSFILILKLGFAIKTRRYLGLRQFGSLHILLMMATQTLLAPSILILVHYGYGTSLNSQLILISYLLVVLSLPVSSIWAATANNSPQLPSSATLSFMNKTTSHFSES

SEQ ID NO: 214 - protein, 417 aa, Candida glabrata
  SITE 1..417: note = Candida glabrata (CgSte2)
MEMGYDPRMYNPRNEYLNFTSVYDVNDTIRFSTLDAIVKGLLRIAIVHGVRLGAIFMTLIIMFISSNTWKKPIFIINMVSLMLVMIHSALSFHYLLSNYSSISYILTGFPQLITSNNKRIQDAASIVQVLLVAAIEASLVFQIHVMFTIENIKLIREIVLSISIAMGLATVATYLAAAIKLIRGLHDEVMPQTHLIFNLSIILLASSINFMTFILVIKLFFAIRSRRYLGLRQFDAFHILLIMFCQSLLIPSVLYIIVYAVDSRSNQDYLIPIANLFVVLSLPLSSIWANTSNNSSRSPKYWKNSQTNKSNGSFVSSISVNSDSQNPLYKKIVRFTSKGDTTRSIVSDSTLAEVGKYSMQDVSNSNFECRDLDFEKVKHTCENFGRISETYSELSTLDTTALNETRLFWKQQSQCDK

SEQ ID NO: 215 - protein, 384 aa, unidentified
  REGION 1..384: note = Vanderwaltozyma polyspora 1 (Vp1Ste2)
MSSQSHPPLIDLFYDSSYDPGESLIYYTSIYGNNTYITFDELQTIVNKKVTQGILFGVRCGAAFLMLVAMWLISKNKRSRIFITNQCCLVFMIMHSGLYFRYLLSRYGSVTFILTGFQQLLTRNDIHIYGATDFIQVALVACIELSLIFQIKVIFAGTNYGKLANYFITLGSLLGLATFGMYMLTAINGTIKLYNNEYDPNQRKYFNISTILLASSINMLTLILILKLVAAIRTRRYLGLKQFDSFHILLIMSTQTLIIPSILFILSYSLREDMHTDQLIIIGNLIVVLSLPLSSMWASSLNNSSKPTSLNTDFSGPKSSEEGTAISLLSQNMEPSIVTKYTRRSPGLYPVSVGTPIEKEASYTLFEATDIDFESSSNDITRTS

SEQ ID NO: 216 - protein, 452 aa, Zygosaccharomyces bailii
  SITE 1..452: note = Zygosaccharomyces bailii (ZbSte2)
MSGLANNTSYNPLESFIIFTSVYGGDTMVKFEDLQLVFTKRITEGILFGVKVGAASLTMIVMWMISRRRTSPIFIMNQLSLVFTILHASFYFKYLLDGFGSIVYTLTLFPQLITSSDLHVFATANVVEVLLVSSIEASLVFQVNVMFAGSNHRKFAWLLVGFSLGLALATVALYFVTAVKMIASAYASQPPTNPIYFNVSLFLLAASVFLMTLMLTVKLILAIRSRRFLGLKQFDSFHILLIMSCQTLIAPSVLYILGFILDHRKGNDYLITVAQLLVVLSLPLSSMWATTANDASSGTSMSSKESVYGSDSLYSKSKCSQFTRTFMNRFSTKPTKNDEISDSAFVAVDSLEKNAPQGISEHVCEFPQSDLSDQATSISSRKKEAVVYASTVDEDKGSFSSDINGYTVTNMPLASAASANCENSPCHVPRPYEENEGVVETRKIILKKNVKW

SEQ ID NO: 217 - protein, 587 aa, Saccharomyces cerevisiae
  SITE 1..587: note = Saccharomyces cerevisiae (ScBar1)
MSAINHLCLKLILASFAIINTITALTNDGTGHLEFLLQHEEEMYYATTLDIGTPSQSLTVLFDTGSADFWVMDSSNPFCLPNSNTSSYSNATYNGEEVKPSIDCRSMSTYNEHRSSTYQYLENGRFYITYADGTFADGSWGTETVSINGIDIPNIQFGVAKYATTPVSGVLGIGFPRRESVKGYEGAPNEYYPNFPQILKSEKIIDVVAYSLFLNSPDSGTGSIVFGAIDESKFSGDLFTFPMVNEYPTIVDAPATLAMTIQGLGAQNKSSCEHETFTTTKYPVLLDSGTSLLNAPKVIADKMASFVNASYSEEEGIYILDCPVSVGDVEYNFDFGDLQISVPLSSLILSPETEGSYCGFAVQPTNDSMVLGDVFLSSAYVVFDLDNYKISLAQANWNASEVSKKLVNIQTDGSISGAKIATAEPWSTNEPFTVTSDIYSSTGCKSRPFLQSSTASSLIAETNVQSRNCSTKMPGTRSTTVLSKPTQNSAMHQSTGAVTQTSNETKLELSSTMANSGSVSLPTSNSIDKEFEHSKSQTTSDPSVAEHSTFNQTFVHETKYRPTHKTVITETVTKYSTVLINVCKPTY

SEQ ID NO: 218 - protein, 435 aa, Candida albicans
  SITE 1..435: note = Candida albicans (CaBar1)
MLFTILSLLVPSLAVAVSNTGGAVKLDLTISDEHMYYIENLALGTPPQFISNVIVDSGSSDLMIVDSIYNFSASSSFYNSNQTAIMKYGYGGQFPVYFINETIRSNDWKLSNLSMGLANISDMDSFSGILGIGFTRQELFKTNYSNFPYLLKDQGYTKSVLFSFNGQDQNPSIIFGGIITNIIDGPLVRAPFIKVISFINQLDYWLMPTFTVNQIKLGDTIVSNQKTLYQIDSGTNGFVPPTPVLNNILKILGDDYIQDDNGNIYFDIKYIEGLNITFSVQGYDIGFQLVDIVGDTIERNSTTFVALNVASCDIGYNAYEGLLPNLIFKYHYAIFDYDNAQIYFGKYKNSNGEANVVAVENGYQLPVPTVDVPDVEDTYSVIYIAESETTVTVAPTEVQSTYSSFSISEGTSSNLTSGSTSDDKITSVTSNPQYC

SEQ ID NO: 219 - protein, 507 aa, Schizosaccharomyces pombe
  SITE 1..507: note = Schizosaccharomyces pombe (SpSxa2)
MLSLFLKSLFAIIIIELTIIHALPTYTVHWKCSIQQANTSSASSNQTVQPRQHAAPSSDRIKSLPEFKGSLPELYSGYLEANSDKSLFYTYAPAVVDSETFIVWLQGGPGCAGTLGFFSENGPIEISQSSPSPSLNPESWTNFANMLWLDQPFGTGYSQGQAAYTTTIEEASSDFVNALKSFYQKFPHLMKKKLYLVGESYGSIWSANFAEALLSEPSLNINFMGVGIVSGLTADYETQEQITASIWVEHISKLGYYFNNTSSTISEEFKKRNKECQYDSVLNRLTFPTEQYPIWRPEYNFSTSTSLRKREALDGEDIGNVFNSISGCDLYSLSNFLLYLENSCVITYDVSLDCSFNEYNDPLITYLNREDVRSSLHATKASTALTSGEGVFADGCNFDLYKKIVSNNVESVLVEIIPRLTEKYKVSFLAGALDLQILWTGTLLALQNTTWNGWQGFTQSPGSLETTNGFTLDERNLAFTLSNSVGHMAPSKDPQMVREWLENTLLY

SEQ ID NO: 220 - protein, 509 aa, Schizosaccharomyces octosporus
  SITE 1..509: note = Schizosaccharomyces octosporus (SoSxa2)
MNLAVHVLLLSFLLYTVHSFPAYPPYPIYPRSSLNSSNASSVPIPQPSYNSSSKAMNPDLVTSLPEYSGSLPELYSGYLSAENFKQIFYTFAPAITPTDKFIVWLQGGPGCAGTLGFFSENGPIILDPNHQTPQDNPNSWTRFANMMWIDQPFGTGYSQGSPLASTVSIATSDFIQTLKSFYAKFPEMKEKELYLVGESYGSVWGAYFAQALRSDPELHAIPFGGLGIISGLLSDYETQNTITASMWLDHVNKMGLYYTNESAEVSTFFAAKNQQCGYAEVLERMQFPAEQTPIPKPRVNLTNSFSKKKRQADSDSDSGNIKEAFQMITDCDTYTLTDFLLYLKNHCMITYDITLDCSFNSNNDPLTTYLNRKDVQKQLHAIHVESALTSSKGIFGNGCNYDVYGDIVKNGAPSILEEAIPELVQNYKVSFLAGALDFQLLWTGSLLALQNTTWNGWQGFQEIPDLNSPFGFTLDERNLAFTLSNKVGHMAPSKDPSMVIDWLQKTLLG

SEQ ID NO: 221 - protein, 584 aa, Schizosaccharomyces japonicus
  SITE 1..584: note = Schizosaccharomyces japonicus (SjSxa2)
MVVLHRLLLSLWPAGAMLLVSSFLLNLPTTSAIPIYSDPHQCSLRVSVRTDVEGEQQQTQAMGSVVIASNVTSSDAPTENPTSSAPTTFSHTVNSTFPTHTPTTSTSTNTPTDARVSSLPLLNGDMPALYAGYLNVSASSTSPHSIFYAYAPSEQGSDDLIVWLQGGPGCAGTIGLFAENGPVRFGPGDSAPVRSEHSWTRYADVLYLDQPFGTGFSYAVTEADYTTTMDAASAEFIAFLDGFFDAYPDTASKRLFFVGESYGSFWGAYFARALADRPDIYDRIVSVGIVSGLSTTHDTQYSVSNSLWLSHLNKLGYYTQDKSTWNSFSRVNARCGYDKVMQEFTFPASQTPLSSISTSTSSSSTSSGAGAGATVSSLTDSSGNASYTNSSSSAEVDVFSYFPSCDTNDATYMLMYLQNTCLISYDVSIDCTTASQLTDHLTPYLNRADVREALHVPSSVAAFSTANGVFGDGCNYDLLQKINTVTYEPVLKSVVPSLIADGKRVAFLSGALDYQIPWSGTLLAMQNTTWAGWQGFQTEPDWDNPVGFTLNERGLSFTLLNSVGHMASMNDPDRVNRWLQEQVL

SEQ ID NO: 222 - DNA, 711 bp, unidentified
  misc_feature 1..711: note = mCherry (RFP) sequence
  source 1..711: mol_type = genomic DNA
atggtttcaaaaggtgaagaagataatatggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaatag

SEQ ID NO: 223 - DNA, 720 bp, unidentified
  misc_feature 1..720: note = ymTurquoise2 sequence
  source 1..720: mol_type = genomic DNA
atggttagtaaaggtgaagaattgttcactggtgtagtgccaattttagtagaattagatggtgacgttaacggtcataagttctctgtaagcggtgaaggtgaaggtgacgcaacatacggtaaattaactttgaaattcatttgtactacaggtaaattaccagttccatggccaactttggttactactttgtcttggggtgttcaatgtttcgctagatacccagatcatatgaagcaacatgatttctttaagtctgctatgccagaaggttacgttcaagaaagaactatcttcttcaaggatgatggtaactataagactagagctgaagttaagttcgaaggagatactttagttaatagaattgaattgaagggtattgattttaaggaagatggtaatattttgggtcataagttggaatataattacttctctgataacgtttacattactgctgataagcaaaagaacggtattaaggcgaacttcaagattagacataacattgaagatggtggtgttcaattagctgatcattaccaacaaaacactccaattggtgacggtccagttttgttgccagataaccattacttgtctactcaatctaagttgtctaaggatccaaacgaaaagagagatcatatggttttgttggaattcgttactgctgctgggatcactttaggtatggatgaattgtataaatag

SEQ ID NO: 224 - DNA, 719 bp, unidentified
  misc_feature 1..719: note = sfGFP
  source 1..719: mol_type = genomic DNA
atgtccaagggtgaagagctatttactggggttgtacccattttggtagaactggacggagatgtaaacggacataaattctctgttagaggtgagggcgaaggcgatgccaccaatggtaaattgactctgaagtttatatgcactacgggtaaattacctgttccttggccaaccctagtaacaactttgacatatggtgttcaatgtttctcaagatacccagaccatatgaaaaggcatgatttctttaaaagtgctatgccagaaggctacgtgcaagagagaactatctcctttaaggatgacggtacgtataaaacacgagcagaagtgaaattcgaaggggatacactagttaatcgcatcgaattaaagggtatagactttaaggaagatggtaatattctcggccataaacttgagtataatttcaactcgcataatgtgtacattacagctgacaaacaaaagaacggaattaaagcgaattttaaaatcaggcacaacgtcgaagatgggtctgttcaacttgccgatcattatcagcaaaacacccctattggtgatggtccagtcttgttacccgataatcactacttaagcacacagtctagattgtcaaaagatccgaatgaaaagcgtgatcacatggttttattggaatttgtcaccgctgcaggaataactcacggaatggacgagctttataagggtag

SEQ ID NO: 225 - DNA, 1431 bp, unidentified
  misc_feature 1..1431: note = ytdTomato
  source 1..1431: mol_type = genomic DNA
atggttagtaaaggtgaggaagtgattaaggaattcatgaggtttaaagttagaatggaaggttctatgaatggtcatgaatttgaaattgaaggtgaaggtgaaggtagaccatatgaaggtacacaaactgctaaattgaaagttacaaaaggtggtccattaccatttgcatgggatattttatctccacagtttatgtatggtagtaaagcatatgttaaacatccagctgatattccagattataagaaattaagttttcctgaaggttttaaatgggaaagagtaatgaattttgaagatggtggtttagtaacagttactcaagattcttctttacaagatggtacattaatatataaagttaaaatgagaggtactaattttccacctgatggtccagttatgcagaagaaaacaatgggttgggaagcctcaactgaaagattgtaccctagagatggagttttgaaaggtgaaattcatcaagctcttaagttaaaagatggaggtcactatttggttgagtttaaaactatatacatggctaaaaaacctgttcaattgccaggttattattatgttgatactaaattagatattacttcacataatgaagattatactattgttgaacaatatgaaagatctgaaggtagacatcatttgtttttgggtcatggtactggtagtactggttctggttcttcaggtactgcttctagtgaagataataatatggctgtaatcaaagagttcatgaggtttaaagttagaatggaaggttctatgaatggtcatgagtttgaaatcgaaggtgaaggagaaggtagaccatatgaaggtactcaaactgctaaattgaaagttactaaaggtggtccattgccatttgcttgggatatattgtcacctcagtttatgtatggttctaaagcatatgttaaacatccagctgatattcctgattataaaaaattatcatttcctgaaggttttaaatgggaaagagttatgaattttgaagatggtggtttagttactgtaactcaagattcttcattgcaagatggtacattaatatataaagttaaaatgagaggtactaattttccacctgatggtccagttatgcagaaaaagactatgggttgggaagcatctactgaaagattatatcctagagatggtgttttaaaaggtgaaattcaccaagccttaaaattgaaagatggtggtcattatttggttgaattcaaaacaatctacatggctaaaaaaccagttcagctgcccggatactattatgtcgatactaaactggatattacttcacacaatgaagactacacaattgttgaacagtatgaaagatcagagggtagacatcacttgtttttatacggtatggatgaattatataaatag

SEQ ID NO: 226 - DNA, 879 bp, synthetic construct
  misc_feature 1..879: note = Synthetic: pTDH3-GPCR-tSTE2
  source 1..879: mol_type = other DNA
ggtctcacagtagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgacggatactagtaaactcgagacggctttgaaaaagtaatttcgtgaccttcggtataaggttactactagattcaggtgctcatcagatgcaccacattctctataaaaaaaaatggtatctttcttatttgataatatttaaactcctttacataataaacatctcgtaagtagtggtagaaaccacctttgcttttacgagttcaccaacgagacc

SEQ ID NO: 227 - DNA, 618 bp, synthetic construct
  misc_feature 1..618: note = Synthetic: LexO(6x)-pLEU2m-readout-tTDH1
  source 1..618: mol_type = other DNA
ggtctcaccaaaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctgctaactcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggcatcgagacc

SEQ ID NO: 228 - DNA, 4310 bp, synthetic construct
  misc_feature 1..4310: note = Synthetic: LexO(6x)-pLEU2m-CrtI-tADH1(tHMG1-pCCW12,rev)
  source 1..4310: mol_type = other DNA
ggtctcaccaaaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctatgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgcgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcaacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacctttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagttttcaccaattggtccatgatctttattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatgggaaattccaaaacaaacggttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattccgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtggcatcgagacc

SEQ ID NO: 229 - DNA, 6281 bp, synthetic construct
  misc_feature 1..6281: note = Synthetic: LexO(6x)-pLEU2m-CrtI-tADH1(tHMG1-pCCW12, rev)_pHHF2-ERG20-tPGK1
  source 1..6281: mol_type = other DNA
ggtctcaccaaaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctatgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgcgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcaacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacctttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagttttcaccaattggtccatgatctttattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatgggaaattccaaaacaaacggtttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattccgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtgcccctgtggagtgtttgcttggatcctttagtaaaaggggaagaacagttggaagggccaaagtggaagtcacaaaacagtggtcctatataaaagaacaagaaaaagattatttatatacaactgcggtcacaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctaaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaataatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagttaaatagaggtttgtccgttgtggacacgtatgctattctctccaacaagaccgttgaacaattggggcaagaagaatacgaaaaggttgctattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaagtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggctatgtacgttgccggtatcacagatgaaaaggatttgaaacaagccagagatgtcttgattccattgggtgaatatttccaaattcaagatgactacttagactgcttcggtaccccagaacagatcggtaagatcggtacagatatccaagataacaaatgttcttgggtaatcaacaaggcattagaacttgcttccgcagaacaaagaaagactttagacgaaaattacggtaagaaggactcagtcgcagaagccaaatgcaaaaagattttcaatgacttgaaaatcgaccagttataccacgaatatgaagagtctgttgccaaggatttgaaggccaagatctcccaagtcgacgagtctcgtggcttcaaagccgacgtcttaactgcgtttttgaacaaggtttacaagagaagtaaataactcgagattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgtgcatcgagacc

SEQ ID NO: 230 - DNA, 600 bp, synthetic construct
  misc_feature 1..600: note = Synthetic: LexO(4x)-pADL6m-pre-pro peptide-tCYC1
  source 1..600: mol_type = other DNA
ggtctcagcatggtagtccatcgttgtaggatactgtatataaatacagtagtggtcatcgatactgtatataaaaccagtaggcaatcgtttgtactgtatgtacatacagtatacctcgcaactactgtatataaacacagtaattaacttgtaatattctaatcaattgatatataaatgtaataagaagtttggtaatattcaattcgaagtgttcagtcttttacttctcttgttttatagaagaaaaaacatcaagaaacatctttaacatacacaaacacatactatcagaatacaagatcttacagacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcgagtcgagacc

SEQ ID NO: 231 - DNA, 973 bp, synthetic construct
  misc_feature 1..973: note = Synthetic: pCCW12-protease-tENO2
  source 1..973: mol_type = other DNA
ggtctcagagtcacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaatttcccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataaagatcactagtactagttaactcgagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaatttttcgagacc

SEQ ID NO: 232 - DNA, 962 bp, synthetic construct
  misc_feature 1..962: note = Synthetic: pRAD27-protease-tENO2
  source 1..962: mol_type = other DNA
ggtctcagagtccttgtgaaattgcaaatatggtgatttgaaacgtttcctagtgcagcaggatcacagataacgtgtaaagggcttagcagttgataatcctctctagttaagacctaaacaaaatgctgtcactaaccgtagtattaaatgacacactttggtgactttcgttaatggggatgtggtagtggccattgccaataaacaaaaagaacagggaaagaagtagaaagtgatataagtttgcttgccacttttcgtttttcacgaaaaaaacaggcgaaaaaaaatgctagacaagtacccggctgaatcacacctcgttaacagtgactttcggtgacagatacccgattgggcacccggctggtaagttatgatagaaagccaacgctgtactattggcttagctatggcaatattttgattatcagctagttttattaacgttataattagtgtaaccagtttttcatctatttcatttatttcatttatttactttaattgcagatccccctaacgcgtttaaagcttttattcactagcttatgtattttttataggaaacgcgacgcgtaacatcgcgcaaatgaaggttttgatgtattataatgaggtattcttccttatatacatcgatgaaaagcgttgacagcatacattggaaagaaataggaaacggacaccggaagaaaaaatagatctactagttaactcgagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaatttttcgagacc

SEQ ID NO: 233 - DNA, 31 bp, synthetic construct
  misc_feature 1..31: note = Synthetic: CCAA-GCAT
  source 1..31: mol_type = other DNA
ggtctcaccaagcgccgggcgcatcgagacc

SEQ ID NO: 234 - DNA, 31 bp, synthetic construct
  misc_feature 1..31: note = Synthetic: GCAT-GAGT
  source 1..31: mol_type = other DNA
ggtctcagcatgcgccgggcgagtcgagacc

SEQ ID NO: 235 - DNA, 31 bp, synthetic construct
  misc_feature 1..31: note = Synthetic: GAGT-TTTT
  source 1..31: mol_type = other DNA
ggtctcagagtgcgccgggcttttcgagacc

SEQ ID NO: 236 - DNA, 31 bp, synthetic construct
  misc_feature 1..31: note = Synthetic: GCAT-TTTT
  source 1..31: mol_type = other DNA
ggtctcagcatgcgccgggcttttcgagacc

SEQ ID NO: 237 - DNA, 5559 bp, synthetic construct
  misc_feature 1..5559: note = Synthetic: pTEF1-CrtE-tADH1-(CrtB-pPGK1,rev), LexO(6x)-pLEU2m-CrtI-tACT1
  source 1..5559: mol_type = other DNA
ggtctcacagtatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggtttctggttcgaaagcaggagtatcacctcatagggaaatcgaagtcatgagacagtccattgatgaccacttagcaggattgttgccagaaacagattcccaggatatcgttagccttgctatgagagaaggtgttatggcacctggtaaacgtatcagacctttgctgatgttacttgctgcaagggacctgagatatcagggttctatgcctacactactggatctagcttgtgctgttgaactgacacatactgcttccttgatgctggatgacatgccttgtatggacaatgcggaacttagaagaggtcaaccaacaacccacaagaaattcggagaatctgttgccattttggcttctgtaggtctgttgtcgaaagcttttggcttgattgctgcaactggtgatcttccaggtgaaaggagagcacaagctgtaaacgagctatctactgcagttggtgttcaaggtctagtcttaggacagttcagagatttgaatgacgcagctttggacagaactcctgatgctatcctgtctacgaaccatctgaagactggcatcttgttctcagctatgttgcaaatcgtagccattgcttctgcttcttcaccatctactagggaaacgttacacgcattcgcattggactttggtcaagcctttcaactgctagacgatttgagggatgatcatccagagacaggtaaagaccgtaacaaagacgctggtaaaagcactctagtcaacagattgggtgctgatgcagctagacagaaactgagagagcacattgactctgctgacaaacacctgacatttgcatgtccacaaggaggtgctataaggcagtttatgcacctatggtttggacaccatcttgctgattggtccccagtgatgaagatcgcctaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttagacaggtctttgccataaaccagcaggtcttggtgtaactctggttgttttggcacgaatgacttgtcctggtgcagccataagcatcgcaatcttctctcctttgctagtgtgttgacgtctatcccaagcagaaccacctgcagcttttaccttgatgccaatctctctgtagacagatcttgcagtagctatagcccaagcacatctaggtggtagatcatgcaatccagcttgacttgagatgtaatagggttcagcagcgtctatcagcctttcagcaactcttgctaatgcagctctgttctctcttgcagcgtaattctcaggagttagaccagcatcttgcaaccattcagcaggtagatagcatctgtcaatagctgcatcgtcgataatatctctcgcgatgtttgtcagctgaaaagccaaacctagatcacaagctctgtccaaaaccctttcgtctcttacacccattactcttgccatcatcaaaccaactactccagcaacatggtagcagtatctcaaggtgtcttcaaaggtcacgtaacgagtttgagcaacatccattgcgaaaccatccaagtgatcaagtgccattcttggcgtaataccgtgtgttagtgcaacttcttggaatgcagcaaaagcaggatcttgcatctcagcaccttcaaaagctgcaagtgtaagcgttctcaatctagccaatctctgagtagcctcttcttctgcagcagcttcagatgcgaaaccatgtgtctggtcatctataacgtcatcacagtgtctacaccaagtgtacagcataagcactgatcttctagtagctgggtcaaacagtttagctgctgtagcaaaggacttggaaccattagccatcgtttgagtagcatgatccaacaaaggtggttgactcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagaaggagggaaacacttcctttttctggccctgataatagtatgagggtgaagccaaaataaaggattcgcgcccaaatcggcatctttaaatgcaggtatgcgatagttcctcactctttccttactcacgagtaattcttgcaaatgcctattatgcagatgttataatatctgtgcgtcttgagttgaagtcaggaatctaaaataaaaattaaggttaataaaaagaggaaagaaaaaaaaattaatcgatttacagaaacttgcacactaaaaatacacaactaaaagcaattacagtatgggaagtcatcgacgttatctctactatagtatattatcatttctattattatcctgctcagtggtacttgcaaaacaagataagaccccattctttgaaggtacttcttcgaaaaattcgcgtctccccaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctaaaaaaatgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaatctctgcttttgtgcgcgtatgtttatgtatgtacctctctctctatttctatttttaaaccaccctctcaataaaataaaaataataaagtatttttaaggaaaagacgtgtttaagcactgactttatctactttttgtacgttttcattgatataatgtgttttgtctctcccttttctacgaaaatttcaaaaattgaccaaaaaaaggaatatatatacgaaaaactattatatttatatatcatagtgtttttcgagacc

SEQ ID NO: 238 - DNA, 6385 bp, synthetic construct
  misc_feature 1..6385: note = Synthetic: pTDH3-FAD1-tPGK1, LexO(6x)-pLEU2m-CrtI-tACT1- [pCCW12-CaBAR1-tENO2, rev]
  source 1..6385: mol_type = other DNA
ggtctcacagtgcgccgggcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaaggattctagaactagtaacatgcagttgagcaaggctgctgagatgtgttatgagataacaaactcttacttacacatagaccagaaatctcagataatagcaagtacacaagaagcgatacggttgacaagaaaatacttactaagtgaaatttttgtacgttggagtccactgaatggggaaatatcattctcgtacaacggaggaaaagattgccaggtattactactgttatatctgagttgcttatgggaatatttcttcattaaggctcaaaattcccaattcgatttcgagtttcaaagcttccccatgcaaagacttccaactgttttcattgatcaagaagaaactttccctacattagagaattttgtactggaaacctcagagcgatattgcctttccttatacgaatcacaaaggcaatctggtgcatcggtcaatatggcagacgcatttagagattttataaagatataccctgaaaccgaagctatagtgataggtattagacacacagacccatttggtgaagcattaaagcctattcaaagaacagattctaactggcctgattttatgaggttgcaacctctcttacactgggacttaaccaatatatggagtttcttactgtattctaatgagccaatttgtggactatatggtaaaggtttcacatcaatcggcggaattaacaactcattgcctaacccacacttgagaaaggactccaataatccagccttgcattttgaatgggaaatcattcatgcatttggcaaggacgcagaaggcgaacgtagttccgctataaacacgtcacctatttccgtggtggataaggaaagattcagcaaataccatgacaattactatcctggctggtatttggttgatgacactttagagagagcaggcaggatcaagaattaaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgccccaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctggaaatgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaatctctgcttttgtgcgcgtatgtttatgtatgtacctctctctctatttctatttttaaaccaccctctcaataaaataaaaataataaagtatttttaaggaaaagacgtgtttaagcactgactttatctactttttgtacgttttcattgatataatgtgttttgtctctcccttttctacgaaaatttcaaaaattgaccaaaaaaaggaatatatatacgaaaaactattatatttatatatcatagtgtatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcactctcgagttaactagtttaacaatactgagggttgcttgtgacggaagtaatcttatcatcacttgtgctgccgctcgttaggttgctagaggtaccctcactgatagaaaaggatgaataggtgctttggacctctgtcggtgcaacggttaccgtggtttctgattccgcgatatatataacgctataagtgtcctcaacatcgggcacgtctactgtcggtactggtagctggtaaccattctccacagcgacgacgttcgcctctccgttagaattcttatatttaccgaaatatatttgtgcgttgtcgtagtcaaatatagcatagtggtacttaaaaatcaaatttggcagcagaccttcataggcgttatagcctatatcgcatgaagctacatttaaggctacgaatgtagttgagttcctttcaatggtatcgccgactatgtcgaccagctggaagccaatgtcgtacccttgtactgaaaatgttatatttaacccctctatgtacttgatgtcgaaatatatattgccattgtcatcctggatgtagtcgtcgcctaaaattttaaggatgttattaaggaccggagttggggggacgaaaccgttagtgccactatcgatttggtaaagcgtcttctggttgctaacgatggtgtctcccaacttgatctgatttactgtgaacgttggcatcaaccagtagtcaagctggtttatgaaggatatcactttgataaagggcgctcttactaaaggcccgtctatgatgttcgtaattatccctccgaagataatggacgggttctgatcctgaccattgaatgagaatagaacacttttcgtataaccctggtctttaagtagataggggaagttactgtaattcgttttaaaaagttcttgacgagtaaatccaatgcctagtataccggaaaaactgtccatatcagaaatattggccagtcccatagatagattagacagtttccagtcgttactcctaatagtttcattgatgaagtataccgggaactgtcccccgtatccatatttcataattgcggtctggttggagttatagaaacttgatgacgcagagaagttgtatatggagtcaactatcataagatcgctacttcctgaatcgactataacgttagatatgaactggggcggcgtccctaacgctaaattctctatgtaatacatgtgctcatcggatatcgtaaggtctaatttcaccgcaccgccggtattggatacggccacggctagggatgggacaagaagtgaaagaatagtaaataacatactagtgatctttattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatgggaaattccaaaacaaacggtttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattccgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtgttttcgagacc

SEQ ID NO: 239 - DNA, 6385 bp, synthetic construct; identical to SEQ ID NO: 238
  misc_feature 1..6385: note = Synthetic: pTDH3-FAD1-tPGK1, LexO(6x)-pLEU2m-CrtI-tACT1- [pCCW12-CaBAR1-tENO2, rev]
  source 1..6385: mol_type = other DNA

SEQ ID NO: 240 - DNA, 1110 bp, unidentified
  misc_feature 1..1110: note = A. clavatus; AclaSte2
  source 1..1110: mol_type = genomic DNA
atgaacagtacgttcgatcccttcgaacaaagcttcactctacaacgttccgatggtaagacgttcaatacttctctggttatcgccgaccataacatggaatatatgctgagactttctattaactacggaacccagttgggagcatcaatcgtcctattactgatactaatgcttctaactcgtccggagaagaggctgagtagcgtattcctactaaacgtggctgcgcttgcatcaaacatcgtcaggctggtatgccagataagctatttttcatctgggtttgcgaaaacctatgctttattagcgcatgatttctccagggtggcccaggctacatacgcaagtcaagttatggcatcagttttttttacgttggtcttggtgtgtgtggaaatttcactagtcctacaagtacaggtcgtttgctctaacttgcgtaggaggtataggcacttacttcttggtgcatccatattggtaacacttataccgatagggtttagattggcttttatggtagagaattgcatcgtaatcgttcgtagtggaaacatgaagaattaccattggttggagtccgctacgaacattgtcgctactgtgtcaatatgttttttttgcgcgatattcgtcgtaaagctgggcttcgcaataaagatgaggcgtcgtctgggggtaaaagattttggccctatgaaggtcatattcattatgggctgccaaaccatgatcatccctgccctattttctatttgtcaatattgggttaacatacctgaattttcacacaacttgctgacgttagtaactatctcactgcctctaagtagtatttgggcaggtttcactctggatagctctaataacacagcgaactccacagctagccgtagaaacctgtggaatgtgttatccagcaatggtacggttcaagacaaaccgaatctttctgcgtgctctccggtagcctcaccaaatacgacctgttatagtgacccttatagcggcaagccgcaccatcaggatctagaggctggatttggcatcagtgtggaacacgatattagcatacactcttatagaaaggacgcacatagtgatgtt

SEQ ID NO: 241 - DNA, 1107 bp, unidentified
  misc_feature 1..1107: note = A. fischeri; AfiSte2
  source 1..1107: mol_type = genomic DNA
atgaactccaccttcgacccatggacccaaaacattactttgactcaatccgacggtaccactgtcatctcctctttggctttggccgatgactacttgcactacatgattagattgggtatcaactacggtgcccaattgggtgcttgtgctgttttgttgttggttttgttattgttgactagaccagaaaagagagtttcttctgtcttcgttttgaacgtcgctgctttgttggctaacatcatcagattgggttgtcaattgtcctacttctctaccggtttcgctagaatgtacgccttgttggccggtgacttctccagagtctctcgtggtgcttacgccggtcaagttatggcctccgtcttcttcaccattgtcttcatttgtgttgaagcttctttggttttgcaagttcaagtcgtctgttctaacttgagaagacaatacagaatcttgttattgggtgcttccactttggctgccttggttccaattggtgttcgtttgacttactccgttttaaactgtatggttattatgcacgctggtactatggaccacttggattggttggaatctgctaccaacatcgttactaccgtttctatttgtttcttctgtgctgttttcgttgtcaaattaggtttggctatcaagatgagaaagcgtttgggtgtcaaacaattcggtccaatgagagttatcttcatcatgggttgtcaaaccatgaccatcccagctattttcgctatttgtcaatacttctctagaattccagaattttctcataacgttttgactttggttatcatctctttgccattgtcttctatctgggccggttttgctttggtccaagccaactctaccgccagatctaccgaatctagacatcatttgtggaacattttgtcttccgatggtgctaccagagacaagccatcccaatgtgtttcttctccaatgacctctccaaccactacctgttactccgaacaatccacctctaagccacaacaagacccagaaaacggttttggtatttctgttgcccacgatatttccatccactctttcagaaaggacgcccacggtgatatt

SEQ ID NO: 242 - DNA, 1107 bp, unidentified
  misc_feature 1..1107: note = A. flavus, A. oryzae; AoSte2
  source 1..1107: mol_type = genomic DNA
atggactctaagttcgacccatactctcaaaacttgactttccacgctgctgacggtaccccatttcaagttccagtcatgaccttgaacgacttttaccaatactgtattcaaatttgtatcaactacggtgctcaattcggtgcttccgtcatcattttcattatcttgttgttattgactagaccagacaaaagagcttcttctgttttcttcttaaacggtggtgccttgttgttgaacatgggtagattgttgtgtcacatgatttacttcactactgacttcgtcaaggcttaccaatacttctcttctgattactctagagccccaacctctgcctacgctaactccattttgggtgtcgtcttgaccaccttgttgttggtttgtatcgaaacctccttggttttacaagtccaagtcgtctgtgctaacttgagacgtagatacagaaccgtcttattgtgtgtttctatcttggtcgccttgatcccagtcggtttgagattgggttacatggttgaaaactgtaagactattgttcaaactgataccccattgtctttggtttggttggaatctgctactaacatcgtcattaccatctccatctgtttcttctgttctatcttcatcatcaagttgggtttcgccattcaccaaagaagaagattgggtgtcagagatttcggtccaatgaaggtcattttcgtcatgggttgtcaaactttgactgttccagctttgttgtctattttgcaatacgctgtctctgtcccagaattgaactctaacattatgactttggttactatctctttgccattgtcctccatttgggctggtgtttctttgacccgttcttcctccaccgaaaactctccatccagaggtgctttgtggaaccgtttgaccgactctaccggtaccagatctaaccaaacctcttccaccgacaccgccgtcgctatgacctacccatctaacaagtcttctactgtctgttacgccgatcaatcttctgtcaagagacaatacgatccagaacaaggtcacggtatctctgttgaacacgatgtttctgtccactcctgtcaaagattg

SEQ ID NO: 243 - DNA, 1107 bp, unidentified; identical to SEQ ID NO: 46
  misc_feature 1..1107: note = A. fumigatus; AfuSte2
  source 1..1107: mol_type = genomic DNA

SEQ ID NO: 244 - DNA, 1107 bp, unidentified
  misc_feature 1..1107: note = A. lentulus; AleSte2
  source 1..1107: mol_type = genomic DNA
atgaattcaacgttcgatccgtggactcaaaatataacgcttactcagagtgatggcacgacagttatatcatctcttgcgctagcggacgactacctgcattacatgatacgtttatccattaattacggtgcacaattaggagcttgcgctgtcctacttctagtccttttgctgttgacaagaccggaaaaaagggtaagtagcgtatttgtactgaatgtagctgctctacttgcgaatataataagacttggatgtcaactttcatacttttctactggtttcgcacgtatgtatgcccttctagcaggcgacttttccagagtctcaaggggagcgtacgcggggcaggtcatggcttcagtttttttcacgatagtactgatctgcgtcgaggcctcactagtgctgcaggttcaggtagtttgtagcaatttgagaagacaatacaggattcttctgttaggcgctagtacgctggccgccttagtaccgataggcgtaaggctgacttattcagttctaaattgcatggtcatcatgcacgcagggactatggaccacttggactggttggagtcagcaactaacatcgtgacgacagtaagcatatgctttttttgcgccgtgttcgtagtcaaactaggtttggcaataaagatgagaaagaggttgggggtcaagcagtttggcccaatgagggtcatcttcattatggggtgtcaaactatgactataccggccatctttgcgatatgtcaatattttagcaaaataccagagtttagccataatgtgctgacccttgtaataatttcactaccgttaagctccatttgggcgggctttgccctagatcaggcaaattctacggcaaggagcagcgagtcacgtcaccatttatggaacattcttagttctgacggagcgacaagggataagccatctcaatgtgtatcatctcccatgaccagtccaaacacaacctgctatagcgagcaatcaacgagtaagccgcagcaagatccagagaacgagtttggcatcagcgtggcgcatgatattagcatccattccttcagaaaggatgcacacggacacttg

SEQ ID NO: 245 - DNA, 1290 bp, unidentified
  misc_feature 1..1290: note = A. nidulans; AnidSte2
  source 1..1290: mol_type = genomic DNA
atggctacccacaaccaaatctctgatcaatgtcaatggtcttacccagaagtcttcaccactcaagctgtcgaagaaccaaccgccgaaccagcttcttaccacttgcactctaccttgactattatggcttctaacttcgacccatggaaccaaaccattaccttcagattggaagacggtactccattcgacatttctgtcgactacttggacggtatcttgcaatactctatcagagcttgtgtcaactacgctgctcaattgggtgcttctgtcattttgtttgttatcttggtcttgttgactagagccgaaaaaagagcttcttgtttgttctggttaaactccttagctttgttgttgaacttcgccagattgttgtgtgacgtcttgttcttcaccggtaacttcgtcagaatttacactttgatctccgctgacgaatctagagttactgcttccgacttggctacttccatcgtcggtgctatcatgaccgctttgttgttgaccactattgaaatttctttggttttgcaagtccaagtcgtttgttctaacttgagaagaatctacagaagagccttgttgtgtgtttccgccgtcgttgccactgctaccattgctattagatactccttgttggctgtcaacattagagctattttggaattctccgacccaactacttacaactggttggaatctttagctaccgtcgccttgaccatctccatctgttacttctgtgtcatcttcgtcaccaagttaggtttcgctattagattgagaagaaagttgggtttatctgaattgggtccaatgaaggtcgtcttcatcatgggttgtcaaaccttggtcatcccaggtaaaagaaccttgtcttctttgattccaccagtcattgtttctattactcactacgtctccgacgtcccagaattgcaaactaacgttttgactatcgtcgccttgtccttgccattgtcctctatttgggctggtaccaccattgacaagccagtcactcactctaacgttagaaacttgtggcaaatcttgtccttctctggttacagaccaaagcaatctacctacattgctaccactactaccgctactaccaacgctaagcaatgtacccactgttactctgaatctagattgttgactgaaaaggaatctggtcgtaacaacgacacttcttctaagtcttcctcccaatacggtatcgctgtcgaacacgatatttccgttagatctgctcgtcgtgaatcttttgacgtc

SEQ ID NO: 246 - DNA, 1134 bp, unidentified
  misc_feature 1..1134: note = A. niger; AnigSte2
  source 1..1134: mol_type = genomic DNA
atggcatcaacggacccgttcgctcaaaatgtaaccttccacactggagacggaaccccatttgaagttaacgtacaagaaattgacacttttctacaatattgtatccaaatttgtaccaattacggggcacaactaggagcatctatagttttatttgtaatattattacttttaacgaggcctgaaaaaagatctagtagtgtgtttatcctgaattgcgcggccttgctttctaatgtagggcgtctattctgccaaatactatacttcaccaccgattttgtaagggtatatgcatacttctccggggatttttcaagggtgccgacttccgcctatgctaactccgtaatagctgtggtcttttatacgattttagtaatctgtattgaagcaagcttggttttacaagttcaggtaatctgcgccaatctgggtagggtctatagacaggtcttattaagtctatcaattgttgttgccctaatacccgtgggtttcagaatggccatgatggtagagaatgccatagccatcatggcggcagagtcttccattccactaatatggctgcaaagcgccgagaatattacggccaccatatcaatttgcttcttttgcgccgtttttgtggccaaattgggatttgcgataagacagagaagaaggcttggcgtccgtgatttcgggcctatgaaagttatatttattatgggctgtcagacattagtcatcccagcgacgtttagtataatacactatatagccgccgtcccagctttatcctctaatgtagcgaccttggtcacgatttcactgccattgagtagtatttgggcaggcgtggcgctgacaaaggtcagctccggtgcggggaccggaagcggtactgggagcggctccagtggaaggaacttgtggaacgtgctgagctttcccgtatgggatagtagaaataccaccagtaccacaacgtcaagttccactccaaccgtgaatccagtgagcacctgttatgcagataacgggagttctaaacaatgcaagcaacaaacgaacactgaggagattgggataacagtcgaacatgagatttctgttcaaagtttcaagagggagaaggacatggtg

SEQ ID NO: 247 - DNA, 1134 bp, unidentified
  misc_feature 1..1134: note = A. novofumigatus; AnoSte2
  source 1..1134: mol_type = genomic DNA
atgaatagcacattcgacccttggacacaaaatattacgctgacgcagagtgacggtactacagtgatatcctccctggcacttgctgatgactatctacattacatgattcgtctgtcaattaactacggagcacaactaggggcgtgtgccgtcttgttattggtactattactgttgacccgtccggagaaacgtatcagtagcgtttttgtgctgaacgtgaccgctttactggcaaatataatcagactgggatgtcaattgtcatatttcagcacggggtttgctcgtatgtacgcactgctggcgggggacttctcaagggtatccagaggagcctacgctggtcaggttatggccagtgtaagtttcacaattgttctgatatgtgtcgaagcaagcttagtactgcaagttcaggtggtatgtagtaatcttagacgtaggtataggatattgctgcttggtgcaagcacccttgccgcactggttccaattggggtgaggctaacttacagcgttcttaattgtatggtaatcatgcatgcaggaactatggatcacttggattggttggagtccgctaccaacattgtaactaccgtctccatttgtttcttctgcgctgtatttgtcattaagctgggcctggccattaaaatgaggaagagattaggtgttaaacaatttggtccgatgagggttatattcattatgggatgtcaaacaatgactatccccgccatattcgccatttgtcagtatttttccagaatcccagagtttagtcataatgttttaaccctggtcatcatctcccttccgctatcttccatttgggcaggattcgcattagaccaggcaaatagtacggcaagatctacggaatccagacaccatctatggaacattcttagcagtgacggcgcgactagagataagcccactcaatgtgtgtcctcaccgatgacttcccccaacaccacgtgctacagcgagcaaagcacaagtaagccccaacaagatcccgagaacggctttggcattagtgtggctcatgatatatccatccacagttttaggaaaggcgcgaacggtatattcgagcaatccctacacctaagaaatgct

SEQ ID NO: 248 - DNA, 1119 bp, unidentified
  misc_feature 1..1119: note = A. terreus; AteSte2
  source 1..1119: mol_type = genomic DNA
atgggcagtactttcgatccattctcccagtccatcactcttaggggtcccgctgaaaacgagatcacgataccgatcgaaaccgttgacttcttttaccagtattgcattagaatttgcataaactacggtgctcagttaggtgcaagcatcgttttatttatcatactactgttgttgaccaggcccgaaaagaggcgttcttccgtatttgtactgaatattacggcgcttctattgaatgtttctagacttatctgccaattgatctattttacttctgaatttgtccgtttatacgcgtatttcggcaacgatttttctagagtgcccccgtccgcgtacgccgattcaatattgggggtcgttcttgtcactctacttattgtttgcatcgaagcgtcattagtcctacaggcacaagtagtttgtgctaatctaagaaggggctaccgtaggattcttttagctgtttcaatagtgatggctttggtcccgataatattcaggtttgtgtacatggtgcaaaattgttggcatataatgcatgcagcagagacaattagactgaattggttagaatcagccacaaatgtagtcatcacgatatctatatgttttttctccgcagttttcataactaagcttggattcgcgatgcatttaagaaagagactgggcatgaccgatttcggtcctatgaaagtaatctttgtgatgggctgccagacgatggctatcccagctatctttagtatattgcagtacatcgtaagcgtacccgaactggcatcaaatatatttagcttagtgactatctccctgcccctatcaagcatctgggcaggcttcacgattgaccaaagagcacgtgattcaggtatgtcaagctccaggaacaacttatttcaggcgttaagcttctctaaagaagcaaagactaggcaaagtagcaccgtcacatatgcttcccaaccgagttccaagagtcacacgctttgttatgcggaactttctcccgctaagcaacaatctgacgcagaagcggcctacgggatcgcagtcgaaagagacatcagcatacattcttgtaggaaagacggaaaggccccgaatgtg

SEQ ID NO: 249 - DNA, 1107 bp, unidentified
  misc_feature 1..1107: note = A. udagawae; AuSte2
  source 1..1107: mol_type = genomic DNA
atgaattccacgttcgatccctggacacagaacattaccctgacgcgttctgacgggaccactgtcattagtagcttggcactggctgatgagttcctgagatatatgatccgtttgagtataaattacggagcgcagctgggagcttcagttgtactgcttttggtactgatgctgctgacgcgtcctgaaaagcgtgtcagttccgtcttcatgctaaatgttgctgcgctgctggctaatatcatccgtttgggttgtcaactgagttattttagtacaggcttcgcgaagatgtatgcattactggccggagactttagtcgtgttagtagaggggcgtacgcgggtcaagtgatggcctcagtattctttacgcttgttttaatttgtgtcgaagtaagtctggttttgcaggtccaagtcgtctgcagcaatttacgtcgtcagtacaggatacttctattgaccgctagtacctttgctgcattagtgcctatagctgtcaggctgacgtactccgtacaaaactgcatggtgataatgcatgccggcacgatggaccatctagattggctagaatcagctactaacatcgtcaccactgtctctatctgcttcttttgtgcagttttcgtcgtgaaattgggccttgcgatcaagatgagaaagaggttgggcgtaaaacagtttgggccgatgagagtcatatttatcatgggatgccagacgatgacaatacccgctatttttgcaatttgtcagtactttagtaagatcccagagttctcccataatgttctgacccttgtaataatcagccttcctctatcttcaatttgggcgggcttcgctctggagcaggccaatcctaccgccagaagtacagagagccgtcataatctttggaatgtattgtcctcagatggagctgcgagagacaaaccttcccaatgtatttcctcctcattgacaagcccgaacgcaacatgctattcagaacaaagtacgtcaaaatctgaacaagatcctgcaaataggttcgggattagcgtcgcccacgacatatccatccgtagttatcagaaggatgcacacggcgatgta

SEQ ID NO: 250 - DNA, 1107 bp, unidentified
  misc_feature 1..1107: note = A. viridinutans; AvSte2
  source 1..1107: mol_type = genomic DNA
atgaattccgcattcgacccctggactcagaacattactttaacacgttccgacggtacaaccgttatttcttctttagcactagctgacgaatttttgcgttacatgattagattgagtataaactatggagcacagcttggggcgtgtgtcgtgttactgctggttctgctattgctaactagacccgaaaaaagagtgtcctccgttttcgtcctgaatgtggcggcgctgcttgccaacataatccgtctaggctgtcaacttagttatttcacgaccggctttgctcgtatctatgcgttgttagccggcgacttctctcgtgtgtctcgtggcgcctacgccggccaggttatggctagtgtattcttcacactagtccttatctgcgttgaagtaagtctagttttgcaagtacaggttgtctgctccaatctgagaaggcaatacagaatcttgttattgggcgcaagtacactagccgcattggttccaatcgctgtgaggctaacatactccgtccagaattgcatggtcataatgcacgccggaacgatggatcatctagactggttagagtctgctaccaatatcgtaaccacggtttctgtatgtttcttttgcgcagtgttcgtggtcaagttagggctggcgattaagctgcgtaaaaggcttggtgtaaaacaatttggtccaatgcgtgtaatcttcattatgggttgtcaaactatgacaatcccagctatctttgccatttgtcagtacttttctgagatcccagagttctcccacaacgtattgacattagtcataatcagtttgcccctaagttccatttgggctggcttcactcttgagcaggctaattccacggctagatctacagaaagcagacataacctttggaacatacttagctccgatggtgctgtcagggataaaccgggacaatgtattagtagcccactgacgagtcctaacaccacgtgttactcccaacactctacagggaaacctcagcaagatccggaaaacggtttcggtattgcggttgcccacgatatcagtatccacagctatcaaaaggatgcgaggggtgatgta

SEQ ID NO: 251 - protein, 105 aa, unidentified
  REGION 1..105: note = A. clavatus; accession number: A1CE26 [217]
MKLLSVVLATLAATTVQAGAMAQWCELPGQGCYMIKRAAEASGEVKRSASAVADAVAEAFPDAALWCELPGQGCHKVKRAAEAAEEVKRSADAFAEAMAALEHLE

SEQ ID NO: 252 - protein, 102 aa, unidentified
  REGION 1..102: note = A. fischeri; accession number: A1DM33 [217]
MRLLSLVLATFAATAVQADITPWCHLPGQGCYMLKRAADASDEVRRSASAVAEAVAEAFPQTPWCHLPGQGCAKAKRAAEAAEEVKRSADAFAEAMAAFEKE

SEQ ID NO: 253 - protein, 103 aa, unidentified
  REGION 1..103: note = A. flavus; accession number: B8N2L7 [217]
MKLISVVVAALAATSVQAGVLQKWCSLPAQGCYMLKRAADASGDVRRSAEALAEAMPDAEALAKWCALPGQGCLKAKRAAEAVEEARRSADALADAMADLGEY

SEQ ID NO: 254 - protein, 110 aa, unidentified
  REGION 1..110: note = A. oryzae; accession number: A0A1S9DPJ9
MKLISVVVAALAATSVQAGVLQKWCSLPAQGCYMLKRAADASGDVRRSAEALSEAMPDAEALAKWCALPGQGCLKAKRAAEAVEEARRSADALADAMADLGESLPRALSF

SEQ ID NO: 255 - protein, 102 aa, unidentified
  REGION 1..102: note = A. fumigatus; accession number: Q4WNH4 [217]
MKLLSLVLASFAAAAVQAHITPWCHLPGQGCYMLKRAADASDEVRRSASAVAEAVAEAFPQSPWCHLPGQGCAKAKRAAEAAEEVKRSADAFAEAMAAFEKE

SEQ ID NO: 256 - protein, 102 aa, unidentified
  REGION 1..102: note = A. lentulus; accession number: A0A0S7DHY4
MKLLSLVLATFAATAVQADITPWCHLPGQGCYMLKRAADASDEVRRSASAVAEAIAEAFPQTPWCHLPGQGCAKAKRAAEAAEEVKRSADAFAEAMAAFEKE

SEQ ID NO: 257 - protein, 128 aa, unidentified
  REGION 1..128: note = A. nidulans; accession number: Q7SI74 [217]
MKLFFVSILLAALLATAVKAAPAAELQHRWCRFAGRICPPTKRTADALNFVKREAEAVAEPFKINRWCRFRGQVCGKAKRAAEAIGNVKLSAEAVADAMAFLDELTREEYAQLAKDFGHLKESDNSDG

SEQ ID NO: 258 - protein, 108 aa, unidentified
  REGION 1..108: note = A. niger; accession number: A2RBH8 [217]
MKLVSLVLAALAATTVQAAALQRWCVLPGQPCNMIKRAADASGEVKRSADALAEAIADATPETLQRWCVLPGQPCNKIKRAVEAGSEVKRSADAFAEAMADIDESDFQ

SEQ ID NO: 259 - protein, 102 aa, unidentified
  REGION 1..102: note = A. novofumigatus; accession number: A0A2I1CDQ5
MKLLSLVLATFAATAVQADITPWCHLPGQGCYMLKRAADASDEVRRSASAVAEAVAEAFPQTPWCHLPGQGCAKAKRAAEAAEEVKRSADAFAEAMAAFEKE

SEQ ID NO: 260 - protein, 136 aa, unidentified
  REGION 1..136: note = A. terreus; accession number: Q0CFX7 [217]
MKLITLVLATLAATSSVQAAKMHKWCWLPGQGCYMLKRAADVTGEVKRHAEALAEAMPDTSPDGIHKWCWLPGQGCLKVKRAADAVDEVKRSADALAEAMAAIEELHAEDLVEIAIGDKEDAAMCDSDYVLSFDIR

SEQ ID NO: 261 - protein, 102 aa, unidentified
  REGION 1..102: note = A. udagawae; accession number: A0A0K8LJ87
MKLLSLVLATLAATAVQADITPWCHLPGQGCYMLKRAADASDEVRRSASAVAEAVAEAFPQTPWCHLPGQGCAKAKRAAEAAEEVKRSADAFANAMAAFEEE

SEQ ID NO: 262 - protein, 102 aa, unidentified
  REGION 1..102: note = A. viridinutans; accession number: XP_043129994
MKLLSLVLATLAATAVQANVTPWCHLPGQGCYMLKRAADASDEVRRSASAVAEAVAEAFPQTPWCHLPGQGCAKAKRAAEAAEEVKRSADAFADSMAAFEKE

SEQ ID NO: 263 - protein, 13 aa, synthetic construct
  REGION 1..13: note = Synthetic: CaPep2A13A
GARLTNFGYFEPA

SEQ ID NO: 264 - DNA, 1479 bp, synthetic construct
  misc_feature 1..1479: note = Synthetic: CrtI gene sequence
  source 1..1479: mol_type = other DNA
atgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaa

SEQ ID NO: 265 - DNA, 1141 bp, synthetic construct
  misc_feature 1..1141: note = Synthetic: pDR04_mSsSte2 (yDR05)
  source 1..1141: mol_type = other DNA
tataccgaaggtcacgaaattactttttcaaagccgtctcgagctagctttcagaaaagtgagaggtcgttttgttcatgaatgaaagagttgcggaagatggaagttgaggagaattgttggcagttgctgcccagatagaggatactggtaaagacaaaacaacaagcaagtaacttataagaatgagctggctattcagagatgtgccatatccgtaatgtacaagaatgagaatagatggggccaataatgtttgagtagccatcatcaataagatgtgcaaactgccaaattgccgaaggccaaggtatcttcttgtctttatggcaaaccctaacttgagaatcaagatgaaagacattatgctaaccgaagtagaaaaaagtatcgtaggcacatctctaacccaagaccaggtagcgaccgttggcgtaggagttccattgacaactgaatgataaattctcgattgttggattgtactattgatagtaaatcctacagcagcagcacccgtgaatgcagatatggaggtaagagctatacccaacttcttcacttctggtgattggaagacgacgaacacctgaaatgtcatggacgcctcgataagagcaacaacaataacctggaaagcattggtggcttcagagacagagaaattggtttctggagttaccaagccggagaacgtaaaacttaatccagccaaaggacctagcaaatatgccaaattaagagcagaccgaataatcattgcagtcaacgtaaccgaattaagatagaaagctggcgacttcttgcgtttgacattgaagatcaaaataattaaaagcattatggtgcatgaaccaatggaaactccatggataatggccttttgcatagattggttaacatattcgtcaaaagctccaaatgggacactaattactctaggatcatttggcaaggtgtagttgacaatgatattcgcagggttgagagtattgatactagtatccattttactagtatccgtcgaaactaagttcttggtgttttaaaactaaaaaaaagactaactataaaagtagaatttaagaagtttaagaaatagatttacagaattacaatcaatacctaccgtctttatata

SEQ ID NO: 266 - DNA, 1130 bp, synthetic construct
  misc_feature 1..1130: note = Synthetic: pDR05_mSsSte2 (yDR06)
  source 1..1130: mol_type = other DNA
ggtcacgaaattactttttcaaagccgtctcgagctagctttcagaaaagtgagaggtcgttttgttcatgaatgaaagagttgcggaagatggaagttgaggagaattgttggcagttgctgcccagatagaggatactggtaaagacaaaacaacaagcaagtaacttataagaatgagctggctattcagagatgtgccatatccgtaatgtacaagaatgagaatagatggggccaataatgtttgagtagccatcatcaataagatgtgcaaactgccaaattgccgaaggccaaggtatcttcttgtctttatggcaaaccctaacttgagaatcaagatgaaagacattatgctaaccgaagtagaaaaaagtatcgtaggcacatctctaacccaagaccaggtagcgaccgttggcgtaggagttccattgacaactgaatgataaattctcgattgttggattgtactattgatagtaaatcctacagcagcagcacccgtgaatgcagatatggaggtaagagctatacccaacttcttcacttctggtgattggaagacgacgaacacctgaaatgtcatggacgcctcgataagagcaacaacaataacctggaaagcattggtggcttcagagacagagaaattggtttctggagttaccaagccggagaacgtaaaacttaatccagccaaaggacctagcaaatatgccaaattaagagcagaccgaataatcattgcagtcaacgtaaccgaattaagatagaaagctggcgacttcttgcgtttgacattgaagatcaaaataattaaaagcattatggtgcatgaaccaatggaaactccatggataatggccttttgcatagattggttaacatattcgtcaaaagctccaaatgggacactaattactctaggatcatttggcaaggtgtagttgacaatgatattcgcagggttgagagtattgatactagtatccattttactagtatccgtcgaaactaagttcttggtgttttaaaactaaaaaaaagactaactataaaagtagaatttaagaagtttaagaaatagatttacagaattacaatcaatacctaccgtctttata

SEQ ID NO: 267 - DNA, 1132 bp, synthetic construct
  misc_feature 1..1132: note = Synthetic: pDR06_mSsSte2 (yDR07)
  source 1..1132: mol_type = other DNA
tcacgaaattactttttcaaagccgtctcgagctagctttcagaaaagtgagaggtcgttttgttcatgaatgaaagagttgcggaagatggaagttgaggagaattgttggctgttgctgcccagatagaggatactggtaaagacaaaacaacaagcaagtaacttataagaatgagctggttattcagagatgtgccatatccgtaatgtacaagaatgagaatagatggggccaataatgtttgagtagccatcatcaataagatgtgcaaactgccaaattgccgaaggccaaggtatcttcttgtctttatggcaaaccctaacttgagaatcaagatgagagacattatgataaccgaagtagaaaaaagtatcgtaggcacatctctaacccaagaccaggtagcgaccgttggcgtaggagttccattgacaactgaatgataaattctcgattgatggattgtactattgatagtaaatcctacagcagcagcacccgtgaatgcagatatggaggtaagagctatacccaacttctccacttctggtgattggaagacgacgaacacctgaaatgtcatggacgcctcgataagagcaacaacaataacctggaaagcattggtggcttcagagacagagaaattggtttctggagttaccaagccggagaacgtaaaacttaatccagccaacggacctagcaaatatgccaaattaagagcagaccgaataatcattgcagtcaacgtaaccgaattaagatagaaagctggcgacttcttgcgtttgacattgaagatcaaaataattaaaagcattatggtgcatgaaccaatggaaactccatggataatggccttttgcatagattggttaacatattcgtcaaaagctccaaatgggacactaattactctaggatcatttggcaaggtgtagttgacaagaaaatccgcagggttgagagtattgatactagtatccattttactagtatccgtcgaaactaagttcttggtgttttaaaactaaaaaaaagactaactataaaagtagaatttaagaagtttaagaaatagatttacagaattacaatcaatacctaccgtctttatatact

SEQ ID NO: 268 - DNA, 1107 bp, synthetic construct
  misc_feature 1..1107: note = Synthetic: pDR07_mSsSte2 (yDR08)
  source 1..1107: mol_type = other DNA
cgtctcgagctagctttcagaaaagtgagaggtcgttttgttcatgaatgaaagagttgcggaagatggaagttgaggagaattgttggcagttgctgcccagatagaggatactggtaaagacaaaacaacaagcaagtaacttataagaatgagctggctattcagagatgtgccatatccgtaatgtacaagaatgagaatagatggggccaataatgtttgagtagccatcatcaataagatgtgcaaactgccaaattgccgaaggccaaggtatcttcttgtctttatggcaaaccctaacttgagaatcaagatgaaagacattatgctaaccgaagtagaaaaaagtatcgtaggcacatctctaacccaagaccaggtagcgaccgttggcgtaggagttccattgacaactgaatgataaattctcgattgttggattgtactattgatagtaaatcctacagcagcagcacccgtgaatgcagatatggaggtaagagctatacccaacttcttcacttctggtgattggaagacgacgaacacctgaaatgtcatggacgcctcgataagagcaacaacaataacctggaaagcattggtggcttcagagacagagaaattggtttctggagttaccaagccggagaacgtaaaacttaatccagccaaaggacctagcaaatatgccaaattaagagcagaccgaataatcattgcagtcaacgtaaccgaattaagatagaaagctggcgacttcttgcgtttgacattgaagatcaaaataattaaaagcattatggtgcatgaaccaatggaaactccatggataatggccttttgcatagattggttaacatattcgtcaaaagctccaaatgggacactaattactctaggatcatttggcaaggtgtagttgacaatgatattcgcagggttgagagtattgatactagtatccattttactagtatccgtcgaaactaagttcttggtgttttaaaactaaaaaaaagactaactataaaagtagaatttaagaagtttaagaaatagatttacagaattacaatcaatacctaccgtctttatata

SEQ ID NO: 269 - protein, 13 aa, Saccharomyces cerevisiae
WHWLQLKPGQPMY

SEQ ID NO: 270 - protein, 14 aa, Saccharomyces castellii
NWHWLRLDPGQPLY

SEQ ID NO: 271 - protein, 13 aa, unidentified
  REGION 1..13: note = Vanderwaltozyma polyspora2
WHWLRLRYGEPIY

SEQ ID NO: 272 - protein, 14 aa, unidentified
  REGION 1..14: note = Vanderwaltozyma polyspora2
PWHWLRLRYGEPIY

SEQ ID NO: 273 - protein, 13 aa, unidentified
  REGION 1..13: note = Vanderwaltozyma polyspora1
WHWLELDNGQPIY

SEQ ID NO: 274 - protein, 11 aa, Torulaspora delbrueckii
GWMRLRLGQPL

SEQ ID NO: 275 - protein, 11 aa, Torulaspora delbrueckii
GWMRLRLGQPM

SEQ ID NO: 276 - protein, 11 aa, Torulaspora delbrueckii
GWMRLRIGQPL

SEQ ID NO: 277 - protein, 13 aa, Saccharomyces kluyveri
WHWLSFSKGEPMY

SEQ ID NO: 278 - protein, 14 aa, Saccharomyces kluyveri
PWHWLSFSKGEPMY

SEQ ID NO: 279 - protein, 13 aa, Kluyveromyces lactis
WSWITLRPGQPIF

SEQ ID NO: 280 - protein, 15 aa, Kluyveromyces lactis
SPWSWITLRPGQPIF

SEQ ID NO: 281 - protein, 12 aa, Zygosaccharomyces rouxii
HFIELDPGQPMF

SEQ ID NO: 282 - protein, 13 aa, Zygosaccharomyces rouxii
AHFIELDPGQPMF

SEQ ID NO: 283 - protein, 12 aa, Zygosaccharomyces bailii
HLVRLSPGAAMF

SEQ ID NO: 284 - protein, 12 aa, Zygosaccharomyces bailii
PLVRLSPGAAMF

SEQ ID NO: 285 - protein, 13 aa, Zygosaccharomyces bailii
APLVRLSPGAAMF

SEQ ID NO: 286 - protein, 13 aa, Zygosaccharomyces bailii
AHLVRLSPGAAMF

SEQ ID NO: 287 - protein, 13 aa, Candida glabrata
WHWVRLRKGQGLF

SEQ ID NO: 288 - protein, 13 aa, Candida glabrata
WHWVKIRKGQGLF

SEQ ID NO: 289 - protein, 12 aa, Ashbya gossypii
WFRLSLHHGQSM

SEQ ID NO: 290 - protein, 12 aa, Scheffersomyces stipitis
WHWTSYGVFEPG

SEQ ID NO: 291 - protein, 13 aa, Scheffersomyces stipitis
PWHWTSYGVFEPG

SEQ ID NO: 292 - protein, 13 aa, unidentified
  REGION 1..13: note = Komagataella (Pichia) pastoris
FRWRNNEKNQPFG

SEQ ID NO: 293 - protein, 16 aa, unidentified
  REGION 1..16: note = Candida (Pichia) guilliermondii
KKNSRFLTYWFFQPIM

SEQ ID NO: 294 - protein, 13 aa, Candida parapsilosis
KPHWTTYGYYEPQ

SEQ ID NO: 295 - protein, 14 aa, Candida auris
KWGWLRFFPGEPFV

SEQ ID NO: 296 - protein, 14 aa, Yarrowia lipolytica
WRWFWLPGYGEPNW

SEQ ID NO: 297 - protein, 14 aa, unidentified
  REGION 1..14: note = Candida (Clavispora) lusitaniae
KWKWIKFRNTDVIG

SEQ ID NO: 298 - protein, 13 aa, unidentified
  REGION 1..13: note = Candida (Clavispora) lusitaniae
WGWIHFLNTDVIG

SEQ ID NO: 299 - protein, 15 aa, unidentified
  REGION 1..15: note = Candida (Clavispora) lusitaniae
PKWKWIKFRNTDVIG

SEQ ID NO: 300 - protein, 13 aa, Candida albicans
GFRLTNFGYFEPG

SEQ ID NO: 301 - protein, 15 aa, Candida tropicalis
KFKFRLTRYGWFSPN

SEQ ID NO: 302 - protein, 13 aa, Candida tenuis
FSWNYRLKWQPIS

SEQ ID NO: 303 - protein, 12 aa, Lodderomyces elongisporous
WMWTRYGRFSPV

SEQ ID NO: 304 - protein, 15 aa, Lodderomyces elongisporous
DPGWMWTRYGRFSPV

SEQ ID NO: 305 - protein, 17 aa, Geotrichum candidum
GDWGWFWYVPRPGDPAM

SEQ ID NO: 306 - protein, 18 aa, Geotrichum candidum
PGDWGWFWYVPRPGDPAM

SEQ ID NO: 307 - protein, 13 aa, Baudoinia compniacensis
GWIGRCGVPGSSC

SEQ ID NO: 308 - protein, 23 aa, Schizosaccharomyces octosporus
TYEDFLRVYKNWWSFQNPDRPDL

SEQ ID NO: 309 - protein, 27 aa, Schizosaccharomyces octosporus
PACTTYEDFLRVYKNWWSFQNPDRPDL

SEQ ID NO: 310 - protein, 10 aa, Tuber melanosporum
WTPRPGRGAY

SEQ ID NO: 311 - protein, 9 aa, Aspergillus oryzae
WCALPGQGC

SEQ ID NO: 312 - protein, 23 aa, Schizosaccharomyces pombe
TYADFLRAYQSWNTFVNPDRPNL

SEQ ID NO: 313 - protein, 24 aa, Schizosaccharomyces pombe
KTYADFLRAYQSWNTFVNPDRPNL

SEQ ID NO: 314 - protein, 9 aa, unidentified
  REGION 1..9: note = Aspergillus (Neosartorya) fischeri
WCHLPGQGC

SEQ ID NO: 315 - protein, 10 aa, Pseudogymnoascus destructans
FCWRPGQPCG

SEQ ID NO: 316 - protein, 10 aa, Pseudogymnoascus destructans
FCQRPGQLCG

SEQ ID NO: 317 - protein, 12 aa, Pseudogymnoascus destructans
LEFGGLEKEQNS

SEQ ID NO: 318 - protein, 23 aa, Schizosaccharomyces japonicus
VSDRVKQMLSHWWNFRNPDTANL

SEQ ID NO: 319 - protein, 27 aa, Schizosaccharomyces japonicus
PERRVSDRVKQMLSHWWNFRNPDTANL

SEQ ID NO: 320 - protein, 9 aa, Paracoccidioides brasiliensis
WCTRPGQGC

SEQ ID NO: 321 - protein, 16 aa, Mycosphaerella graminicola
GNSFVGWCGAIGAPCA

SEQ ID NO: 322 - protein, 10 aa, Mycosphaerella graminicola
WCGAIGAPCA

SEQ ID NO: 323 - protein, 9 aa, Penicillium chrysogenum
WCGHIGQGC

SEQ ID NO: 324 - protein, 10 aa, Penicillium chrysogenum
KWCGHIGQGC

SEQ ID NO: 325 - protein, 10 aa, Aspergillus nidulans
WCRFRGQVCG

SEQ ID NO: 326 - protein, 15 aa, Phaeosphaeria nodorum
KYNGWRYRPYGLPVG

SEQ ID NO: 327 - protein, 10 aa, Hypocrea jecorina
WCYRIGEPCW

SEQ ID NO: 328 - protein, 10 aa, Hypocrea jecorina
WCWILGGKCW

SEQ ID NO: 329 - protein, 9 aa, Botrytis cinerea
WCGRPGQPC

SEQ ID NO: 330 - protein, 10 aa, Beauvaria bassiana
WCMRPGQPCW

SEQ ID NO: 331 - protein, 9 aa, Beauvaria bassiana
WCMQTPKCW

SEQ ID NO: 332 - protein, 11 aa, Neurospora crassa
QWCRIHGQSCW

SEQ ID NO: 333 - protein, 14 aa, Neurospora crassa
QVCNMRLHPKKVCW

SEQ ID NO: 334 - protein, 10 aa, Sporothrix scheckii
YCPLKGQSCW

SEQ ID NO: 335 - protein, 12 aa, Sporothrix scheckii
QRYCPLKGQSCW

SEQ ID NO: 336 - protein, 11 aa, Magnaporthe oryzea
QWCPRRGQPCW

SEQ ID NO: 337 - protein, 8 aa, Dactylellina haptotyla
WCVYNSCP

SEQ ID NO: 338 - protein, 10 aa, Fusarium graminearum
WCWWKGQPCW

SEQ ID NO: 339 - protein, 10 aa, Fusarium graminearum
WCTWKGQPCW

SEQ ID NO: 340 - protein, 14 aa, Capronia coronata
GLSYWKGVNDGGSS

SEQ ID NO: 341 - DNA, 879 bp, synthetic construct; identical to SEQ ID NO: 226
  misc_feature 1..879: note = Synthetic: pTDH3- GPCR- tSTE2
  source 1..879: mol_type = other DNA

SEQ ID NO: 342 - DNA, 618 bp, synthetic construct
  misc_feature 1..618: note = Synthetic: LexO(6x)-pLEU2m-readout-tTDH1
  source 1..618: mol_type = other DNA
ggtctcaccaaaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctgctaactcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggcattgagacc

SEQ ID NO: 343 - DNA, 973 bp, synthetic construct
  misc_feature 1..973: note = Synthetic: pCCW12-protease-tENO2
  source 1..973: mol_type = other DNA
ggtctcagagtcacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaatttcccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataaagatcactagtactagttaactcgagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaattttttgagacc

SEQ ID NO: 344 - DNA, 31 bp, synthetic construct
  misc_feature 1..31: note = Synthetic: CCAA-GCAT
  source 1..31: mol_type = other DNA
ggtctcaccaagcgccgggcgcattgagacc

SEQ ID NO: 345 - DNA, 31 bp, synthetic construct
  misc_feature 1..31: note = Synthetic: GCAT-GAGT
  source 1..31: mol_type = other DNA
ggtctcagcatgcgccgggcgagttgagacc

SEQ ID NO: 346 - DNA, 31 bp, synthetic construct
  misc_feature 1..31: note = Synthetic: GAGT-TTTT
  source 1..31: mol_type = other DNA
ggtctcagagtgcgccgggctttttgagacc

SEQ ID NO: 347 - DNA, 31 bp, synthetic construct
  misc_feature 1..31: note = Synthetic: GCAT-TTTT
  source 1..31: mol_type = other DNA
ggtctcagcatgcgccgggctttttgagacc

SEQ ID NO: 348 - DNA, 5559 bp, synthetic construct; identical to SEQ ID NO: 237
  misc_feature 1..5559: note = Synthetic: pTEF1- CrtE-tADH1- (CrtB-pPGK1, rev), LexO(6x)-pLEU2m-CrtI-tACT1
  source 1..5559: mol_type = other DNA

SEQ ID NO: 349 - DNA, 6376 bp, synthetic construct
  misc_feature 1..6376: note = Synthetic: pTDH3- FAD1-tPGK1, LexO(6x)-pLEU2m-CrtI-tACT1- [pCCW12-CaBAR1-tENO2, rev]
  source 1..6376: mol_type = other DNA
ggtctcacagtagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaaggattctagaactagtaacatgcagttgagcaaggctgctgagatgtgttatgagataacaaactcttacttacacatagaccagaaatctcagataatagcaagtacacaagaagcgatacggttgacaagaaaatacttactaagtgaaatttttgtacgttggagtccactgaatggggaaatatcattctcgtacaacggaggaaaagattgccaggtattactactgttatatctgagttgcttatgggaatatttcttcattaaggctcaaaattcccaattcgatttcgagtttcaaagcttccccatgcaaagacttccaactgttttcattgatcaagaagaaactttccctacattagagaattttgtactggaaacctcagagcgatattgcctttccttatacgaatcacaaaggcaatctggtgcatcggtcaatatggcagacgcatttagagattttataaagatataccctgaaaccgaagctatagtgataggtattagacacacagacccatttggtgaagcattaaagcctattcaaagaacagattctaactggcctgattttatgaggttgcaacctctcttacactgggacttaaccaatatatggagtttcttactgtattctaatgagccaatttgtggactatatggtaaaggtttcacatcaatcggcggaattaacaactcattgcctaacccacacttgagaaaggactccaataatccagccttgcattttgaatgggaaatcattcatgcatttggcaaggacgcagaaggcgaacgtagttccgctataaacacgtcacctatttccgtggtggataaggaaagattcagcaaataccatgacaattactatcctggctggtatttggttgatgacactttagagagagcaggcaggatcaagaattaaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgccccaaacgggtagtccatcgttgtaggatactgtatatacacccagtagagtaggtgactactgtatgagcatacagtagtgacaaccacttactgtatataaatacagtagtggtcatcgatactgtatataaaaccagtaggcaatcgtttgtactgtatgtacatacagtatacctcgcaactactgtatataaacacagtaattaacttgtaatattctaatcaattgacaatattatttaaggacctattgttttttccaataggtggttagcaatcgtcttactttctaacttttcttaccttttacatttcagcaatatatatatatatatttcaaggatataccattctaagatctggaaatgaagaaaaccgtagtgattggtgcaggttttggtggtttagctttggctatacgtctacaagctgcaggtattcctacagtgctattggagcaaagagacaaaccaggaggaagagcttatgtttggcacgatcaaggctttacctttgatgctggtcctacagtcatcactgatcctactgcattggaagctttgttcaccttagctggtagaagaatggaagattatgtccgtctattgcctgtcaagccgttttacagattgtgttgggaatctggtaaaaccctagattacgccaatgacagtgctgaactagaagctcagattacgcagtttaatcccagagatgtcgaaggttacaggagattccttgcctattcccaagctgttttccaagagggttatcttcgtttgggttcagttccattcctgtcctttagggatatgcttagagcaggtcctcagttgttgaagctacaagcatggcaaagtgtgtatcagtctgtttcgagatttatcgaggatgaacatctgagacaagcattctcattccacagtcttctagttggaggtaatccctttaccacatcgagcatatatacgttgattcacgctttggaaagagaatggggagtttggtttcctgaaggtggaacaggtgctttggttaatggtatggtgaagctattcacggatttgggtggagaaatagagctgaatgcaagagtggaagaacttgttgtagcagacaacagagtctcacaagttagacttgctgatggtaggatcttcgatacagatgctgtagcttcaaacgcagatgtagtgaacacttataaaaagttgttgggacatcatcctgttggacaaaagagagcagctgctttggagaggaaatctatgagcaactcgttgtttgtcctttactttgggctgaatcaaccacactcacaactagctcatcacacaatctgctttggtcctagatacagagagctgatagatgaaattttcactggatctgctttagcagacgatttttccctgtacttgcattcaccatgtgttactgatccctctttagcaccacctggttgtgctagcttctatgtactagcacctgtaccacatttgggtaatgctccattagattgggcacaagaaggaccgaaattgagggataggatcttcgactatttggaagaacgttacatgccaggtttgagatctcagttggttacacagaggatattcacaccagctgattttcatgatactctagatgcgcatttaggtagcgctttttccattgagccacttttgacgcaaagtgcttggtttagaccacacaacagagattctgacattgccaatctgtacctagtaggtgcaggaactcatccaggagctggtattcctggagttgtagcttctgctaaagctactgctagtctgatgatcgaggatttgcagtaatctctgcttttgtgcgcgtatgtttatgtatgtacctctctctctatttctatttttaaaccaccctctcaataaaataaaaataataaagtatttttaaggaaaagacgtgtttaagcactgactttatctactttttgtacgttttcattgatataatgtgttttgtctctcccttttctacgaaaatttcaaaaattgaccaaaaaaaggaatatatatacgaaaaactattatatttatatatcatagtgtatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcactctcgagttaactagtttaacaatactgagggttgcttgtgacggaagtaatcttatcatcacttgtgctgccgctcgttaggttgctagaggtaccctcactgatagaaaaggatgaataggtgctttggacctctgtcggtgcaacggttaccgtggtttctgattccgcgatatatataacgctataagtgtcctcaacatcgggcacgtctactgtcggtactggtagctggtaaccattctccacagcgacgacgttcgcctctccgttagaattcttatatttaccgaaatatatttgtgcgttgtcgtagtcaaatatagcatagtggtacttaaaaatcaaatttggcagcagaccttcataggcgttatagcctatatcgcatgaagctacatttaaggctacgaatgtagttgagttcctttcaatggtatcgccgactatgtcgaccagctggaagccaatgtcgtacccttgtactgaaaatgttatatttaacccctctatgtacttgatgtcgaaatatatattgccattgtcatcctggatgtagtcgtcgcctaaaattttaaggatgttattaaggaccggagttggggggacgaaaccgttagtgccactatcgatttggtaaagcgtcttctggttgctaacgatggtgtctcccaacttgatctgatttactgtgaacgttggcatcaaccagtagtcaagctggtttatgaaggatatcactttgataaagggcgctcttactaaaggcccgtctatgatgttcgtaattatccctccgaagataatggacgggttctgatcctgaccattgaatgagaatagaacacttttcgtataaccctggtctttaagtagataggggaagttactgtaattcgttttaaaaagttcttgacgagtaaatccaatgcctagtataccggaaaaactgtccatatcagaaatattggccagtcccatagatagattagacagtttccagtcgttactcctaatagtttcattgatgaagtataccgggaactgtcccccgtatccatatttcataattgcggtctggttggagttatagaaacttgatgacgcagagaagttgtatatggagtcaactatcataagatcgctacttcctgaatcgactataacgttagatatgaactggggcggcgtccctaacgctaaattctctatgtaatacatgtgctcatcggatatcgtaaggtctaatttcaccgcaccgccggtattggatacggccacggctagggatgggacaagaagtgaaagaatagtaaataacatactagtgatctttattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatgggaaattccaaaacaaacggtttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattccgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtgttttcgagacc

SEQ ID NO: 350 - DNA, 720 bp, unidentified; identical to SEQ ID NO: 223
  misc_feature 1..720: note = ymTurquoise2
  source 1..720: mol_type = genomic DNA

SEQ ID NO: 351 - DNA, 1479 bp, unidentified; identical to SEQ ID NO: 264
  misc_feature 1..1479: note = CrtI
  source 1..1479: mol_type = genomic DNA

SEQ ID NO: 352 - DNA, 20 bp, synthetic construct
  misc_feature 1..20: note = Synthetic: STE2 gene in yWS890
  source 1..20: mol_type = other DNA
tcacctctactgatgaactg

SEQ ID NO: 353 - DNA, 20 bp, synthetic construct
  misc_feature 1..20: note = Synthetic: URA3 gene in yWS890
  source 1..20: mol_type = other DNA
gaagtaacaaaggaacctag

SEQ ID NO: 354 - DNA, 20 bp, synthetic construct
  misc_feature 1..20: note = Synthetic: LEU2 gene in yWS890
  source 1..20: mol_type = other DNA
tttttgaacacacatgaaca

SEQ ID NO: 355 - DNA, 20 bp, synthetic construct
  misc_feature 1..20: note = Synthetic: ARS208a locus
  source 1..20: mol_type = other DNA
gtccgctaaacaaaagatct

SEQ ID NO: 356 - DNA, 20 bp, synthetic construct
  misc_feature 1..20: note = Synthetic: HO locus
  source 1..20: mol_type = other DNA
agaattgattgctgcttatg

SEQ ID NO: 357 - DNA, 20 bp, synthetic construct
  misc_feature 1..20: note = Synthetic: LEU2 locus
  source 1..20: mol_type = other DNA
taacattaatattgacaagg

SEQ ID NO: 358 - protein, 587 aa, Saccharomyces cerevisiae; identical to SEQ ID NO: 217
  SITE 1..587: note = Saccharomyces cerevisiae [35] (ScBar1, NP_012249)

SEQ ID NO: 359 - protein, 435 aa, Candida albicans; identical to SEQ ID NO: 218
  SITE 1..435: note = Candida albicans [26] (CaBar1, KGQ96729)

SEQ ID NO: 360 - protein, 507 aa, Schizosaccharomyces pombe; identical to SEQ ID NO: 219
  SITE 1..507: note = Schizosaccharomyces pombe [36] (SpSxa2, NP_593043)

SEQ ID NO: 361 - protein, 509 aa, Schizosaccharomyces octosporus; identical to SEQ ID NO: 220
  SITE 1..509: note = Schizosaccharomyces octosporus (SoSxa2, XP_013015964)

SEQ ID NO: 362 - protein, 584 aa, Schizosaccharomyces japonicus; identical to SEQ ID NO: 221
  SITE 1..584: note = Schizosaccharomyces japonicus (SjSxa2, XP_002172242)

SEQ ID NO: 363 - protein, 431 aa, Saccharomyces cerevisiae; identical to SEQ ID NO: 186
  SITE 1..431: note = Saccharomyces cerevisiae [37] (ScSte2, D6VTK4)

SEQ ID NO: 364 - protein, 469 aa, Candida albicans; identical to SEQ ID NO: 187
  SITE 1..469: note = Candida albicans [14] (CaSte2)

SEQ ID NO: 365 - protein, 348 aa, Schizosaccharomyces pombe; identical to SEQ ID NO: 188
  SITE 1..348: note = Schizosaccharomyces pombe [14] (SpSte2, NP_594722)

SEQ ID NO: 366 - protein, 348 aa, Schizosaccharomyces octosporus; identical to SEQ ID NO: 189
  SITE 1..348: note = Schizosaccharomyces octosporus [14] (SoSte2, XP_013018801)

SEQ ID NO: 367 - protein, 346 aa, Schizosaccharomyces japonicus; identical to SEQ ID NO: 190
  SITE 1..346: note = Schizosaccharomyces japonicus [14] (SjSte2, XP_002173203)

SEQ ID NO: 368 - DNA, 1296 bp, synthetic construct; identical to SEQ ID NO: 177
  misc_feature 1..1296: note = Synthetic: ScSte2
  source 1..1296: mol_type = other DNA

SEQ ID NO: 369 - DNA, 1410 bp, synthetic construct; identical to SEQ ID NO: 179
  misc_feature 1..1410: note = Synthetic: CaSte2
  source 1..1410: mol_type = other DNA

SEQ ID NO: 370 - DNA, 1047 bp, synthetic construct; identical to SEQ ID NO: 180
  misc_feature 1..1047: note = Synthetic: SpSte2
  source 1..1047: mol_type = other DNA

SEQ ID NO: 371 - DNA, 1047 bp, synthetic construct; identical to SEQ ID NO: 178
  misc_feature 1..1047: note = Synthetic: SoSte2
  source 1..1047: mol_type = other DNA

SEQ ID NO: 372 - DNA, 1041 bp, synthetic construct
  misc_feature 1..1041: note = Synthetic: SjSte2
  source 1..1041: mol_type = other DNA
atgtactcctgggacgaattcagatccccaaagcaagctgaagttttgaaccaaaccgttaccttggaaactattgtttccaccattcaattgccaatctctgaaattgactccatggaaagaaacagattgttgaccggtatgactgtcgctgttcaagttggtttaggttccttcattttagttttgatgtgtattttctcttcctctgaaaagagaaagaagccagtcttcatcttcaacttcgctggtaacttggttatgactttgagagctattttcgaagttatcgttttggcttctaacaactactctatcgctgttcaatacggtttcgcttttgctgccgtcagacaatacgttcacgccttcaacattatcatcttgttgttgggtccattcatcttgttcatcgctgaaatgtctttgatgttgcaagttagaatcatttgttcccaacacagaccaactatgattaccaccactgttatctcttgtattttcactgttgttaccttggccttctggatcaccgacatgtctcaagaaattgcttaccaattgttcttgaaaaactacaacatgaagcaaattgttggttactcctggttgtactttatcgctaagatcaccttcgctgcttccattatcttccattcctccgtcttctccttcaaattgatgcgtgctatttacattcgtagaaagatcggtcaattcccattcggtccaatgcaatgtatcttcattgtttcctgtcaatgtttgatcgttccagctattttcactttgatcgattctttcacccacacttacgatggtttctcctccatgactcaatgtttgttgatcatctccttaccattgtcttccttgtgggccacccacaccgctcaaaagttgcaaaccatgaaggataacactaacccaccatctggtacccaattaaccatcagagttgatcgtactttcgacatgaagttcgtttccgactcctctgacggttctttcactgaaaagaccgaagaaactttgccatag

SEQ ID NO: 373 - DNA, 1764 bp, synthetic construct
  misc_feature 1..1764: note = Synthetic: ScBar1
  source 1..1764: mol_type = other DNA
atgtctgcaattaatcatctttgtttgaaacttattttggcgagtttcgcgattattaacaccattactgctttaacaaacgatggcactggtcacttagaattccttttacaacacgaagaggagatgtattacgcaacaaccttagatataggtacaccgtcccaaagtctgacagtgttgtttgataccggatctgccgatttttgggttatggattctagcaatcccttctgcttaccaaattcaaatacgtcatcctattcaaacgcaacttataatggcgaagaagttaagccttcaattgattgcaggtctatgagtacttataatgagcatagatcttccacctaccaatatctggaaaatggtaggttttacatcacatatgctgacggaacatttgctgacggtagttgggggacggaaactgtatcaattaatggaattgacatccccaatatccagttcggagttgccaagtatgctacgacacccgttagtggtgttcttggaattgggtttcctagaagagagtccgttaagggctatgaaggtgctcctaatgaatattatcctaattttcctcagattttaaaaagtgaaaaaataatcgatgtggtcgcgtattcgctgttcttaaactcacctgattcaggtactggttcgattgtttttggtgccattgatgaatcaaagttttctggtgatttgttcactttccctatggtaaatgaatatcccacaatagtcgacgctcctgcaactttagcaatgactatacaaggattaggtgcccaaaacaaaagtagttgtgaacatgaaacgtttacgacgaccaagtatccagttttgttggactcaggaacctcgctattgaatgcgcccaaggtcatagcagataaaatggcttcttttgtaaatgcgtcctatagtgaagaggaaggtatatatatattagactgtccagtatctgtaggtgacgtggaatacaattttgatttcggcgatttgcaaataagtgttccactgtctagtttgattttaagtcccgagacagaaggcagctattgtgggtttgcggtccagccaacaaacgattcgatggttctgggtgatgtgttcctgtcctctgcatacgtcgtattcgatctcgataattataagatatctttagcacaggcaaattggaacgcaagcgaagtttcgaaaaagctagtaaatattcaaacagatgggtctatttcaggtgccaaaattgctacagctgaaccctggtccaccaatgaaccatttacagtcacctctgacatttattcatctacaggctgcaagagtaggccttttcttcaatcatcgacagcctcttcgcttattgcagaaaccaacgtacaaagtcgcaactgctctacgaagatgccaggcactagatcaactactgtcttaagtaagcctactcaaaatagtgctatgcatcaaagtacaggcgctgtcacacaaacctcaaatgaaactaaattagaattatcctcgactatggcaaattcgggcagtgtctcgcttcccacttcgaattcaatagacaaagagttcgaacattcgaaatctcaaactaccagcgatccaagtgtagcagagcattctacgtttaaccaaacgtttgtacatgaaactaaatatcggcctactcataagacagtcataacagaaactgtcacgaagtattctacagtcttaataaatgtctgtaaaccaacatattaa

SEQ ID NO: 374 - DNA, 1308 bp, synthetic construct
  misc_feature 1..1308: note = Synthetic: CaBar1
  source 1..1308: mol_type = other DNA
atgttatttactattctttcacttcttgtcccatccctagccgtggccgtatccaataccggcggtgcggtgaaattagaccttacgatatccgatgagcacatgtattacatagagaatttagcgttagggacgccgccccagttcatatctaacgttatagtcgattcaggaagtagcgatcttatgatagttgactccatatacaacttctctgcgtcatcaagtttctataactccaaccagaccgcaattatgaaatatggatacgggggacagttcccggtatacttcatcaatgaaactattaggagtaacgactggaaactgtctaatctatctatgggactggccaatatttctgatatggacagtttttccggtatactaggcattggatttactcgtcaagaactttttaaaacgaattacagtaacttcccctatctacttaaagaccagggttatacgaaaagtgttctattctcattcaatggtcaggatcagaacccgtccattatcttcggagggataattacgaacatcatagacgggcctttagtaagagcgccctttatcaaagtgatatccttcataaaccagcttgactactggttgatgccaacgttcacagtaaatcagatcaagttgggagacaccatcgttagcaaccagaagacgctttaccaaatcgatagtggcactaacggtttcgtccccccaactccggtccttaataacatccttaaaattttaggcgacgactacatccaggatgacaatggcaatatatatttcgacatcaagtacatagaggggttaaatataacattttcagtacaagggtacgacattggcttccagctggtcgacatagtcggcgataccattgaaaggaactcaactacattcgtagccttaaatgtagcttcatgcgatataggctataacgcctatgaaggtctgctgccaaatttgatttttaagtaccactatgctatatttgactacgacaacgcacaaatatatttcggtaaatataagaattctaacggagaggcgaacgtcgtcgctgtggagaatggttaccagctaccagtaccgacagtagacgtgcccgatgttgaggacacttatagcgttatatatatcgcggaatcagaaaccacggtaaccgttgcaccgacagaggtccaaagcacctattcatccttttctatcagtgagggtacctctagcaacctaacgagcggcagcacaagtgatgataagattacttccgtcacaagcaaccctcagtattgttaa

SEQ ID NO: 375 - DNA, 1524 bp, synthetic construct
  misc_feature 1..1524: note = Synthetic: SpSxa2
  source 1..1524: mol_type = other DNA
atgttaagtcttttcttaaagtctttgttcgcaattattatcattgaacttactatcatccacgcccttccgacgtacacggtgcactggaagtgtagcatacaacaagccaatacttcctcagcatcctctaaccagaccgtccagccgcgtcagcacgcagcgccgagtagcgatcgtataaagtccctacccgaatttaagggatcactgcctgagttatactcaggttacttggaggcaaattctgacaagtcactattttacacatatgcgcctgcagttgtagactctgaaacgttcatcgtgtggttgcagggcggaccgggctgcgcgggcactctaggtttcttcagtgaaaacggcccgattgagattagccagtcatctccttctccttcattaaatcctgagagttggaccaatttcgcaaacatgttgtggctggaccagccttttggtactgggtatagccaggggcaggcggcttacactaccaccatagaggaagcctctagcgacttcgtcaatgccctaaaatctttttaccaaaaattcccgcatttgatgaaaaagaagctgtatcttgttggggaaagctatggcagtatatggagtgccaattttgcggaggccctacttagcgaaccaagtctaaatatcaattttatgggtgtgggtattgtcagcggactaacagccgattatgagactcaggaacaaataacggcaagtatatgggttgagcatatatccaaactgggttactattttaacaatacctcctctaccatatcagaagagttcaagaaacgtaacaaagagtgtcagtacgactccgtattgaatagattaacgtttcccaccgaacagtatcccatatggcgtcctgaatacaatttttcaacctcaaccagtcttagaaagagggaagcactggatggggaagatatcggtaacgttttcaattccatctctggttgtgatctgtattcattatctaactttctactgtaccttgaaaacagttgcgtgattacatacgacgtcagtcttgactgctccttcaatgaatataacgatccattgattacatacttgaacagagaagacgtcagatcctccttgcacgcaaccaaggcctccacggcgttaacatccggagagggtgtcttcgccgatggatgtaacttcgatctgtataagaagattgtctcaaacaatgtcgaatctgtgctagtagagatcatacctcgtttgacagagaaatataaagtcagtttcttagcaggtgctctggacctacaaattctatggacggggactttgttagctctacagaacactacatggaacgggtggcaaggatttacacaatctcccggctctctggaaacgacgaatggtttcacgctggatgagagaaatcttgcgtttaccctaagtaacagcgtgggccatatggctccaagcaaggatccacaaatggtgagagaatggctagagaacacccttctttactaa

SEQ ID NO: 376 - DNA, 1530 bp, synthetic construct
  misc_feature 1..1530: note = Synthetic: SoSxa2
  source 1..1530: mol_type = other DNA
atgaacttagccgtccatgtactgctgttgtctttcttgttgtacactgttcacagtttccccgcatatccgccatatccaatatatccgagatcatctctgaactcaagtaacgcctccagcgtaccaataccacaaccctcatacaattcatcttccaaggccatgaatcctgacctggtcactagcttgccagagtattcaggtagtctaccagaattatacagcggataccttagcgccgagaacttcaagcagatcttttatacttttgcacctgccattaccccgactgacaaattcatagtttggctgcaagggggtcctgggtgcgcaggcaccttaggcttcttttccgaaaacggtccaattatcttggaccccaatcaccaaactccacaagacaatcccaatagctggactcgtttcgcaaacatgatgtggattgaccaaccctttggaacagggtattctcaaggctccccattggcaagtacagtttcaatcgctacttcagattttatccaaactctgaagtctttttacgcaaaattccctgagatgaaggaaaaagagttgtatcttgtgggcgagtcctatggatccgtgtggggcgcgtattttgcccaagccttgaggtccgatcccgagctgcatgctataccttttggggggctaggtatcataagcggccttctatcagactatgaaactcagaatactataacagcgtcaatgtggcttgatcatgttaacaaaatgggcttgtattatactaatgagtccgccgaggtatctacgtttttcgctgcaaagaatcaacaatgtggttacgcagaagtcctagagagaatgcaattcccagccgaacagactccgatacctaagccgcgtgtcaatttgactaactctttctcaaaaaagaaaaggcaggccgactctgactctgactccggcaatataaaggaagcatttcaaatgatcacggactgtgatacttatacgttaacggactttcttctttacctgaaaaaccattgtatgataacatacgatattacgcttgattgctcattcaattcaaacaacgaccctctaacaacctatctaaatagaaaagacgtacagaagcaactgcacgctattcatgtcgagagcgctttaacatcctccaaaggcatctttggtaatggatgcaactatgacgtctatggtgacattgtcaaaaatggcgcaccttcaatactggaagaagcgatacctgagttagtacagaactataaagtcagtttcttagccggggccttagactttcaattgctttggactggtagtttgctggcactacaaaacacgacctggaacggttggcagggttttcaagagatccctgacctaaactccccctttggttttacgcttgacgagcgtaatctggcgttcacgcttagcaacaaggtaggacatatggccccgagcaaggaccccagcatggtaatagattggctgcaaaaaaccctgctgggttga

SEQ ID NO: 377 - DNA, 1755 bp, synthetic construct
  misc_feature 1..1755: note = Synthetic: SjSxa2
  source 1..1755: mol_type = other DNA
atggtagttttgcacagattgcttttaagtttgtggcccgcaggagccatgttgctagtgtcttcatttctattgaacctgccaacgacatccgcgattccgatttatagcgatccccaccaatgttcacttagggttagcgttagaacagatgtcgagggggagcaacaacagactcaggcaatgggctctgtcgttattgcgtcaaatgtaacgtcatccgacgcgccgaccgaaaaccccacgtctagtgcgccaacgacttttagtcacactgttaacagtacatttcctacccatacgccaacgaccagtacctctacaaatacgcccacggacgcaagagtcagctctttacctttattgaacggcgacatgcccgcactgtatgccggctaccttaacgtaagcgcaagctcaacgtccccacattctattttctacgcgtatgcaccttctgaacaagggtccgatgatttgatcgtatggctgcaaggtgggccgggatgcgcaggcactattggacttttcgccgaaaatgggccggtacgtttcggtcctggcgacagcgctcccgttcgttcagaacactcttggactagatacgcagatgtactttatttggaccaaccattcgggacgggattttcttacgctgtcaccgaagctgactatacgaccacaatggatgcggcgtctgcagaattcatagcatttttggacggattcttcgacgcatacccagatacagcttctaagaggctatttttcgtgggggagtcttacggaagtttttggggggcctactttgccagggccctggcagatagacctgacatttatgatcgtatagttagtgtgggcatcgtgtctggactatcaactactcacgatactcagtacagcgtatctaactccttatggttgtctcacttaaataagctaggctattatacccaggataaaagcacctggaattcttttagtagagtcaatgcacgttgcggttacgacaaggtaatgcaggagttcactttcccagcgagtcagacgccgcttagtagtatatctacgagcacctcaagctcaagtaccagttctggagctggagccggagcaacggtttcttccttgaccgattcttcagggaatgcaagttacaccaattcttctagcagcgctgaagtagacgtgttttcatacttcccctcatgcgacacaaacgacgcaacgtacatgctaatgtatttgcagaatacgtgtttaatttcttatgacgtctctatcgactgtacaactgcttcccaacttacagatcacttaacgccatacttgaatagggcagacgttagggaggccttgcatgtcccctcctctgtggctgcatttagcacggcaaacggtgtgtttggagacggctgcaactatgatttactgcagaaaattaatacagttacctacgaaccagttctgaaatcagtcgtaccgagcttgatcgccgatggtaaaagggtggcctttctgtcaggcgcattggactatcagattccgtggagcggaacccttttggcgatgcaaaatactacttgggccggttggcaaggatttcagacggagcctgactgggacaaccccgtaggctttacgctaaacgaaagaggattgagttttaccttacttaattctgtgggacacatggcctcaatgaatgatccagacagggtgaaccgttggttgcaggaacaagtcctttga

SEQ ID NO: 378 - protein, 13 aa, synthetic construct
  REGION 1..13: note = Synthetic: ScPep1A
AHWLQLKPGQPMY

SEQ ID NO: 379 - protein, 13 aa, synthetic construct
  REGION 1..13: note = Synthetic: ScPep2A
WAWLQLKPGQPMY

SEQ ID NO: 380 - protein, 13 aa, synthetic construct
  REGION 1..13: note = Synthetic: ScPep3A
WHALQLKPGQPMY

SEQ ID NO: 381 - protein, 13 aa, synthetic construct
  REGION 1..13: note = Synthetic: ScPep4A
WHWAQLKPGQPMY

SEQ ID NO: 382 - protein, 13 aa, synthetic construct
  REGION 1..13: note = Synthetic: ScPep5A
WHWLALKPGQPMY

SEQ ID NO: 383 - protein, 13 aa, synthetic construct
  REGION 1..13: note = Synthetic: ScPep6A
WHWLQAKPGQPMY

SEQ ID NO: 384 - protein, 13 aa, synthetic construct
  REGION 1..13: note = Synthetic: ScPep7A
WHWLQLAPGQPMY

SEQ ID NO: 385 - protein, 13 aa, synthetic construct
  REGION 1..13: note = Synthetic: ScPep8A
WHWLQLKAGQPMY

SEQ ID NO: 386 - protein, 13 aa, synthetic construct
  REGION 1..13: note = Synthetic: ScPep9A
WHWLQLKPAQPMY

SEQ ID NO: 387 - protein, 13 aa, synthetic construct
  REGION 1..13: note = Synthetic: ScPep10A
WHWLQLKPGAPMY

SEQ ID NO: 388 - protein, 13 aa, synthetic construct
  REGION 1..13: note = Synthetic: ScPep11A
WHWLQLKPGQAMY

SEQ ID NO: 389 - protein, 13 aa, synthetic construct
  REGION 1..13: note = Synthetic: ScPep12A
WHWLQLKPGQPAY

SEQ ID NO: 390 - protein, 13 aa, synthetic construct
  REGION 1..13: note = Synthetic: ScPep13A
WHWLQLKPGQPMA

SEQ ID NO: 391 - protein, 369 aa, unidentified
  REGION 1..369: note = A. fumigatus Ste2 amino acid sequence encoded by SEQ ID NO:1 and 2
MNSTFDPWTQNITLTQSDGTTVVSSLALADDYLHYMIRLGINYGAQLGACAVLFLVLLLLTRPEKRVSSVFVLNVSALLANIIRLGCQLSYFSTGFARMYALLAGDFSRVSRGAYAGQVMASVFFTIVLICVEASLVLQVQVVCSNLLRQYRILLLGASTLAALVPIGVRLTYSVMNCMVIMHAGTMDHLDWLESATNIVTTVSICFFCAVFVVKLGLAIKMRKRLGVKKFGPMRVIFIMGCQTMTIPAVFAICQYFSRIPEFSHNVLTLVIISLPLSSIWAGFALDQANSTARSTESRHHLWNILSSDGATRDKPSQCANSPMTSPNTTCYSEQSTSKPQQDPENGFGISVAHDISIHSFQKGGHGDI

SEQ ID NO: 392 - DNA, 968 bp, synthetic construct
  misc_feature 1..968: note = Synthetic: pRPL18B-protease-tENO2
  source 1..968: mol_type = other DNA
ggtctcagagtaagaggatgtccaatattttttttaaggaataaggatacttcaagactagattcccccctgcattcccatcagaaccgtaaaccttggcgctttccttgggaagtattcaagaagtgccttgtccggtttctgtggctcacaaaccagcgcgcccgatatggctttcttttcacttatgaatgtaccagtacgggacaattagaacgctcctgtaacaatctctttgcaaatgtggggttacattctaaccatgtcacactgctgacgaaattcaaagtaaaaaaaaatgggaccacgtcttgagaacgatagattttctttattttacattgaacagtcgttgtctcagcgcgctttatgttttcattcatacttcatattataaaataacaaaagaagaatttcatattcacgcccaagaaatcaggctgctttccaaatgcaattgacacttcattagccatcacacaaaactctttcttgctggagcttcttttaaaaaagacctcagtacaccaaacacgttacccgacctcgttattttacgacaactatgataaaattctgaagaaaaaataaaaaaattttcatacttcttgcttttatttaaaccattgaatgatttcttttgaacaaaactacctgtttcaccaaaggaaatagaaagaaaaaatcaattagaagaaaacaaaaaacaaaagatctactagttaactcgagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaatttttcgagacc